(19) United States
(12) Patent Application Publication
(10) Pub. No.: US 2018/0312493 A1
(43) Pub. Date: Nov. 1, 2018

(54) Antibiotic Compounds, Pharmaceutical Formulations Thereof And Methods And Uses Therefor

(71) Applicants: Simon Fraser University, Burnaby, BC (CA); Centre for Drug Research and Development, Vancouver, BC (CA)

(72) Inventors: Robert Norman Young, Vancouver, BC (CA); Nag Sharwan Kumar, Surrey, BC (CA); Alexander Laurence Mandel, Vancouver, BC (CA); Tom Han Hsiao Hsieh, Vancouver, BC (CA); Jason Samuel Tan, Richmond, BC (CA); Fahimeh S. Shidmoossavee, Vancouver, BC (CA); James Brian Jaquith, Cobourg, ON (CA); Edith Mary Dullaghan, Vancouver, BC (CA)

(21) Appl. No.: 15/772,800

(22) PCT Filed: Nov. 4, 2016
(86) PCT No.: PCT/CA2016/000272

§ 371 Date: May 1, 2018

Related U.S. Application Data
(60) Provisional application No. 62/250,510, filed on Nov. 4, 2015.
(60) Provisional application No. 62/278,405, filed on Jan. 13, 2016.

(51) Int. Cl. C07D 405/06; C07D 209/12; C07D 209/18; C07D 209/14; C07D 471/04; C07D 453/02; C07D 401/12; C07D 401/06; C07D 401/14; C07D 209/10; C07D 403/04; C07D 487/14; C07D 409/04; C07D 277/82; C07D 403/14; C07D 487/04; C07D 263/58; C07D 413/04; A61P 31/04

(52) U.S. Cl. CPC C07D 405/06; A61P 31/04; C07D 209/18; C07D 209/14; C07D 471/04; C07D 453/02; C07D 401/12; C07D 401/06; C07D 401/14; C07D 209/10; C07D 403/04; C07D 487/14; C07D 409/04; C07D 277/82; C07D 403/14; C07D 487/04; C07D 263/58; C07D 413/04; C07D 209/12

(57) ABSTRACT

The present invention relates to compounds of formula (I) wherein G¹ to G⁸ are as defined herein. The compounds are PK inhibitors and as such represent a new approach to treating pathogenic infections, including multidrug resistant pathogens. Disclosed herein are the compounds of formula (I), pharmaceutical compositions comprising the compounds of formula (I) and their use in the treatment of antimicrobial infection. (Formula (1))

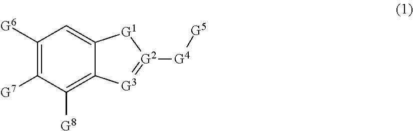

### REFERENCE TO RELATED APPLICATIONS

This application claims the benefit of U.S. Provisional Patent Application Ser. No. 62/250,510 filed on 4 Nov. 2015, and U.S. Provisional Patent Application Ser. No. 62/278,405, filed on 13 Jan. 2016, which are hereby incorporated by reference for all purposes as if fully set forth herein.

### TECHNICAL FIELD

The present invention relates to medicinal chemistry and more particularly antibiotic compounds.

### BACKGROUND

Infectious diseases caused by bacterial and eukaryotic pathogens continue to be a threat to human health. In particular, many bacteria are developing antibiotic resistance and the effectiveness of the available antimicrobial drugs against bacteria such as methicillin-resistant Staphylococcus aureus (MRSA) is diminishing at a rapid pace. The hospital-acquired ESKAPE pathogens (Enterococcus faecium, Staphylococcus aureus, Klebsiella pneumoniae, Acinetobacter baumannii, Pseudomonas aeruginosa and Enterobacter spp.), and others, are recognised as serious community acquired health threats.

Inhibitors of bacterial pyruvate kinase (PK) demonstrate antibacterial activity (Zoraghi et al., 2011, Antimicrob. Agents Chemother. 55:2042-2053). Structural variations between human and bacterial PK, such as MRSA and others, allow for the therapeutic targeting of bacterial PK over human PK. PK catalyzes the final step of glycolysis, which involves the transfer of the phosphoryl group of phosphoenolpyruvate (PEP) to ADP to produce pyruvate and ATP (Suzuki K, et al., 2008, J Biochem, 144(3):305-312). PKs exist as homotetramers of identical subunits of ˜50-60 KDa depending on species, each consisting of three to four domains: A, B, C, and N-terminal domains. The N-terminal helical domain is absent in prokaryotic PKs and can be removed from human erythrocyte PK with no effect on its stability or activity (Valentini et al., 2002, J. Biol. Chem., 277:23807-23814). While there are four mammalian PK isozymes, M1, M2, L (liver), and R (red blood cell), with different primary structures, kinetic properties, and tissue distributions to satisfy the metabolic requirements of various tissues, most bacteria and lower eukaryotes have only one PK isoenzyme. Only a few bacterial species, specifically E. coli and Salmonella typhimurium, have two isoenzymes.

Inhibitors of bacterial PKs identified by structural modelling and in silico library screening have been described (Zoraghi et al., 2011, Antimicrob. Agents Chemother. 55:2042-2053; International Patent Application No. PCT/CA2011/001175 (WO 2012/051708)). A class of MRSA PK inhibitors derived from a naturally occurring marine alkaloid has also been described (Kumar et al., 2014, Bioog. Med. Chem., 22:1708-1725).

Several indole- or benzimidazole-containing compounds have been described as having anti-mycobacterial activity (Matyk et al., 2005, II Farmaco, 60:399-408), antimicrobial activity (International Patent Application No. PCT/US2003/027963 (WO 2005/033065), or broad spectrum anti-bacterial activity (U.S. Pat. No. 8,691,859).

As resistance mechanisms have been reported for most classes of antibacterial therapeutics, new mechanistic targets are required. Inhibitors of PK represent a new approach to treating pathogenic infections, including multidrug resistant pathogens.

This background information is provided for the purpose of making known information believed by the applicant to be of possible relevance to the present invention. No admission is necessarily intended, nor should be construed, that any of the preceding information constitutes prior art against the present invention.

### SUMMARY

The present invention is based, at least in part, on compounds suitable for use as antibiotics.

Illustrative embodiments of the present invention provide a method of treating a subject known to have or suspected of having a bacterial infection, the method comprising administering to the subject an effective amount of a compound having a structure of formula (1):

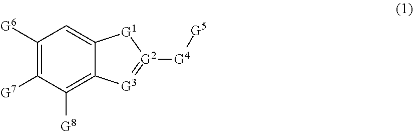

or a salt thereof, wherein: G is NH, O, or S; G², G³ and G⁴ may either: i) together form a ring moiety selected from the group consisting of:

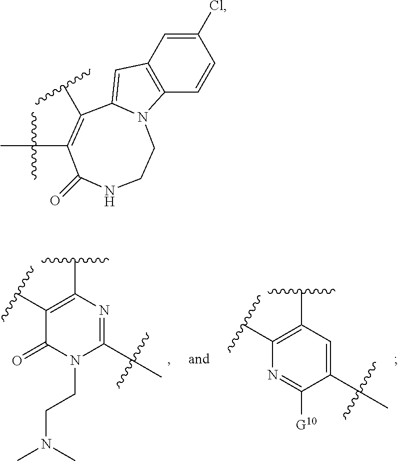

or ii) together do not form a ring moiety wherein G² is C; G³ is N, CH or CG⁹; and G⁴ is selected from the group consisting of: a bond,

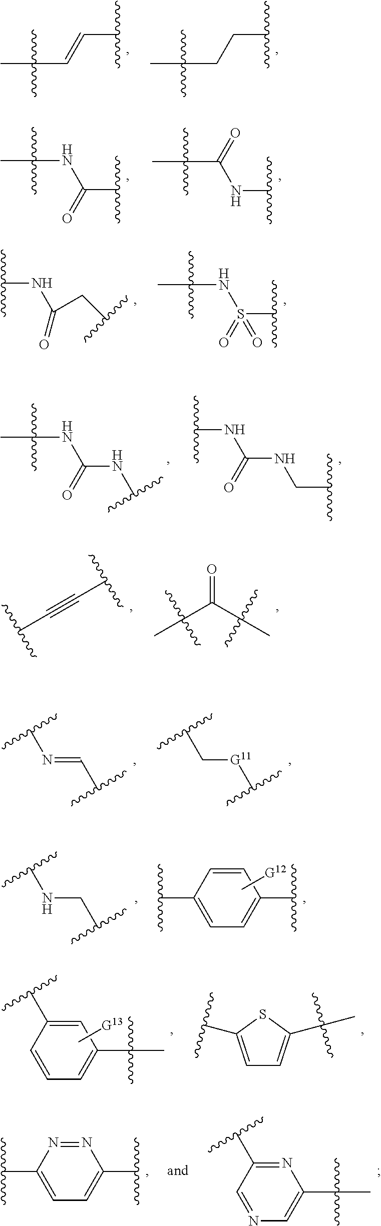

G⁵ is absent,

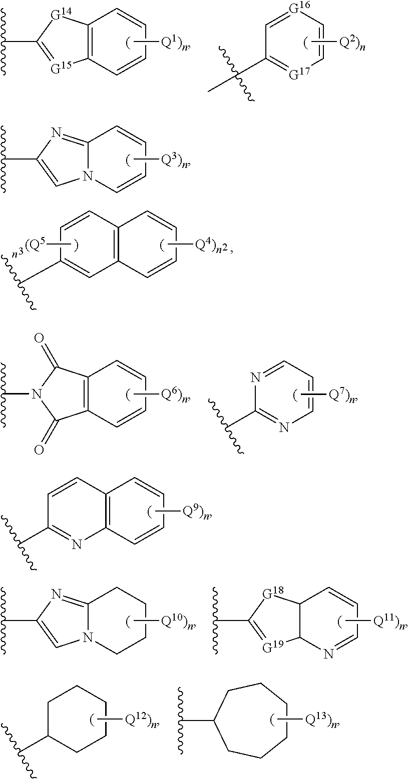

a 5-membered heteroaryl optionally substituted with (Q⁸)ₙ and containing 1 or 2 heteroatoms each heteroatom independently selected from N, O and S, substituted (C₁₋₁₁)alkyl, unsubstituted (C₁₋₁₁)alkyl, substituted (C₁₋₁₁)heteroalkyl, unsubstituted (C₁₋₁₁)heteroalkyl, substituted (C₃₋₁₁)heterocycloalkyl, unsubstituted (C₃₋₁₁)heterocycloalkyl, substituted (C₈₋₉)cycloalkyl, or unsubstituted (C₈₋₉)cycloalky; G⁶ is H, halogen, CF₃, NO₂, substituted (C₁₋₁₁)alkyl, unsubstituted (C₁₋₁₁)alkyl, substituted (C₁₋₁₁)alkoxyl, unsubstituted (C₁₋₁₁) alkoxyl, substituted (C₆₋₁₁)aryloxy, unsubstituted (C₆₋₁₁)aryloxy, C(O)OR⁵⁰, substituted (C₁₋₁₁)heteroalkyl, unsubstituted (C₁₋₁₁) heteroalkyl or

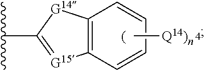

G⁷ is H, halogen, CF₃, NO₂, substituted (C₁₋₁₁)alkyl, unsubstituted (C₁₋₁₁)alkyl, substituted (C₁₋₁₁) alkoxyl, unsubstituted (C₁₋₁₁) alkoxy, substituted (C₆₋₁₁)aryloxy, unsubstituted (C₆₋₁₁)aryloxy, C(O)OR⁵¹, substituted (C₁₋₁₁)heteroalkyl, unsubstituted (C₁₋₁₁) heteroalkyl, or

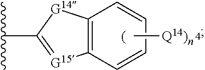

R⁵⁰ and R⁵¹ are each independently substituted (C₁₋₆)alkyl, unsubstituted (C₁₋₆)alkyl, substituted (C₁₋₆)heteroalkyl or unsubstituted (C₁₋₆) heteroalkyl; G⁸ is H, C(═O)N(CH₃)₂, or C(═O)N(H)C(H₂)C₆H₅; G⁹ is —CN, CF₃, —SO₂NH₂, —NH₂, —C(CF₃)₂OH, —C(CF₃)(H)OH, —C(CF₃)(CH₃)OH, —C(NOH)C(R²¹)(R²²)(R²³), C(NOH)N(R²⁴)(R²⁵), C(NOR⁶⁰)C(R⁶¹)(R⁶²)(R⁶³), substituted (C₁₋₆) alkyl-NR⁶⁴R⁶⁵, unsubstituted (C₁₋₆) alkyl-NR⁶⁴R⁶⁵, substituted (C₆₋₁₁) aryl, unsubstituted (C₆₋₁₁)aryl, substituted (C₁₋₁₁) heteroaryl, unsubstituted (C₁₋₁₁) heteroaryl, substituted (C₆₋₁₁) arylcarbonyl, unsubstituted (C₆₋₁₁) arylcarbonyl, substituted (C₁₋₁₁) heteroarylcarbonyl, unsubstituted (C₁₋₁₁) heteroarylcarbonyl, —CO-substituted-carbocycle, —CO-unsubstituted-carbocycle, —CO-substituted-heterocarbocycle, —CO-unsubstituted-heterocarbocycle, —CO-substituted-C(₁₋₆)alkyl-OR¹, —CO-unsubstituted-C(₁₋₆)alkyl-OR¹, —CO-substituted-C(₁₋₆)alkyl-NR²R³, —CO-unsubstituted-C(₁₋₆)alkyl-NR²R³, —CO-substituted-C(₁₋₆)alkyl-C(O)OR⁴, —CO-unsubstituted-C(₁₋₆)alkyl-C(O)OR⁴; —CO-substituted-C(₁₋₆)alkyl-C(O)NR⁵R⁶, —CO-unsubstituted-C(₁₋₆)alkyl-C(O)NR⁵R⁶, —C(O)NR⁷R⁸, —C(O)OR⁹, —C(O)C(O)OR¹², —C(O)C(O)NR¹³R¹⁴, —NR¹⁵R¹⁶, —N(H)C(O)substituted-C(₁₋₆)alkyl, —N(H)C(O)unsubstituted-C(₁₋₆)alkyl, —N(H)C(O)substituted-C(₁₋₆)haloalkyl, —N(H)C(O)unsubstituted-C(₁₋₆)haloalkyl, —N(H)C(O)substituted-C(₆₋₁₁)aryl, —N(H)C(O)unsubstituted-C(₆₋₁₁)aryl, —N(H)C(O)substituted-C(₁₋₁₁)heteroaryl, —N(H)C(O)unsubstituted-C(₁₋₁₁)heteroaryl, —N(H)C(O)NR¹⁷R¹⁸, —N(H)CO-substituted-C(₁₋₆)alkyl-OR¹⁹, —N(H)CO-unsubstituted-C(₁₋₆)alkyl-OR¹⁹, each of R¹, R², R³, R⁴, R⁵, R⁶, R¹², R¹³, R¹⁴, R¹⁵, R¹⁶, R¹⁷, R¹⁸, R¹⁹, R²⁴, and R²⁵ is independently selected from the group consisting of: H, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl, each of R²¹, R²², R²³, R⁶¹, R⁶² and R⁶³ is independently selected from the group consisting of: H, F, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl; each of R⁶⁴ and R⁶⁵ is independently selected from the group consisting of: H, substituted C(₃₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl each pair: a) R² and R³, b) R⁵ and R⁶, c) R¹³ and R¹⁴, d) R¹⁵ and R¹⁶, e) R¹⁷ and R¹⁸, and f) R⁶⁴ and R⁶⁵ may alternately be and independently as a pair be a 3-7 membered substituted heterocarbocyclic ring or a 3-7 membered unsubstituted heterocarbocyclic ring; R⁶⁰ is unsubstituted C(₁₋₁₁)alkyl, substituted C(₁₋₁₁)alkyl, unsubstituted C(₁₋₁₁)alkyl-NR⁶⁶R⁶⁷, substituted C(₁₋₁₁)alkyl-NR⁶⁶R⁶⁷, unsubstituted C(₁₋₁₁)alkyl-NR⁶⁸R⁶⁹R⁷⁰, or substituted C(₁₋₁₁)alkyl-N⁺R⁶⁸R⁶⁹R⁷⁰, wherein R⁶⁶ and R⁶⁷ are each independently H, unsubstituted C(₁₋₁₁)alkyl or substituted C(₁₋₁₁)alkyl, and R⁶⁸, R⁶⁹ and R⁷⁰ are each independently unsubstituted C(₁₋₁₁)alkyl, or substituted C(₁₋₁₁)alkyl, each of R⁷ and R⁸ are either I) independently selected from the group consisting of: H, substituted C(₁₋₆)alkyl, substituted C(₁₋₆)alkyl-NR⁵²R⁵³, unsubstituted C(₁₋₆)alkyl-NR⁵²R⁵³, substituted C(₁₋₆)alkyl-N+R⁷¹R⁷²R⁷³, unsubstituted C(₁₋₆)alkyl-N⁺R⁷¹R⁷²R⁷³, substituted C(₁₋₆)alkyl-OC(O)unsubstituted C(₁₋₆)alkyl-NR⁷⁴R⁷⁵ unsubstituted C(₁₋₆)alkyl-OC(O)unsubstituted C(₁₋₆)alkyl-NR⁷⁴R⁷⁵, substituted C(₁₋₆)alkyl-C(O)NHS(O)₂R⁷⁶, unsubstituted C(₁₋₆)alkyl-C(O)NHS(O)₂R⁷⁶, substituted C(₆₋₁₁)aryl, substituted C(₃₋₁₁)carbocyclic, substituted C(₄₋₇)heterocarbocycle, substituted C(₄₋₇)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₃₋₁₁)carbocyclic, unsubstituted C(₁₋₁₁)heterocarbocycle, unsubstituted C(₁₋₁₁)heteroaryl, unsubstituted C(₇₋₁₁)aralkyl, and unsubstituted C(₂₋₁₁)heteroaralkyl wherein each of R⁵², R⁵³, R⁷⁴ and R⁷⁵ is selected from the group consisting of: H, unsubstituted C(₁₋₆)alkyl, substituted C(₃₋₇)heterocycloalkyl, unsubstituted C(₃₋₇)heterocycloalkyl, substituted C(₁₋₆)alkyl, substituted C(₃₋₇)cycloalkyl and unsubstituted C(₃₋₇)cycloalkyl, or each pair: a) R⁵² and R⁵³, or (b) R⁷⁴ and R⁷⁵, together form a 3-7 membered substituted heterocarbocyclic ring or a 3-7 membered unsubstituted heterocarbocyclic ring, and wherein each of R⁷¹, R⁷², R⁷³ and R⁷⁶ is independently unsubstituted C(₁₋₁₁)alkyl, or substituted C(₁₋₁₁)alkyl, or II) together form a 3-7 membered substituted heterocarbocyclic ring or a 3-7 membered unsubstituted heterocarbocyclic ring; R⁹ is selected from the group consisting of substituted C(₁₋₆)alkyl, substituted C(₁₋₆)alkyl-NR¹⁰R¹¹, unsubstituted C(₁₋₆)alkyl-NR¹⁰R¹¹, substituted C(₁₋₆)alkyl-OR²⁰, unsubstituted C(₁₋₆)alkyl-OR²⁰, and unsubstituted C(₁₋₆)alkyl wherein each of R¹⁰, R¹¹ and R²⁰ is independently selected from the group consisting of: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; R¹⁰ and R¹¹ may alternately as a pair be a 3-7 membered substituted heterocarbocyclic ring or a 3-7 membered unsubstituted heterocarbocyclic ring, or G⁹ is

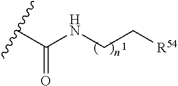

wherein n is 1, 2, 3 or 4 and R⁵⁴ is

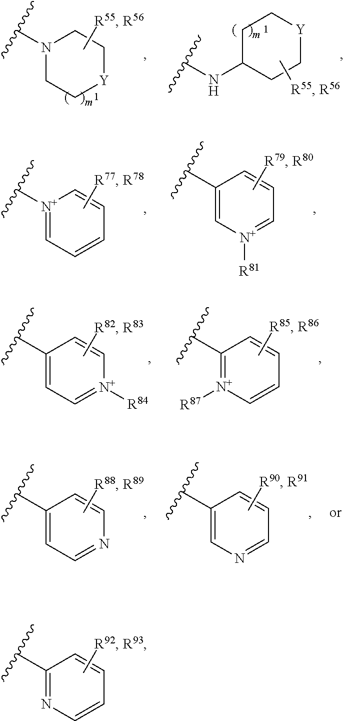

wherein m^{1=0, 1} or 2, R⁵⁵ and R⁵⁶ are independently H, carbonyl (═O), Me, Ph, CO₂R⁹⁴, CO₂NH₂, C(₁₋₆)substituted alkyl or C(₁₋₆)unsubstituted alkyl, wherein R⁹⁴ is H, C(₁₋₆)unsubstituted alkyl or C(₁₋₆)substituted alkyl; R⁷⁷, R⁷⁸, R⁷⁹, R⁸⁰, R⁸², R⁸³, R⁸⁵, R⁸⁶, R⁸⁸, R⁸⁹, R⁹⁰, R⁹¹, R⁹² and R⁹³ are each independently H, C(₁₋₆)substituted alkyl, C(₁₋₆)unsubstituted alkyl, substituted C(₁₋₆)heteroalkyl, unsubstituted C(₁₋₆) heteroalkyl, OR⁹⁵, C(O)R⁹⁶, or NR⁹⁷R⁹⁸, wherein R⁹⁵ is H, C(₁₋₆)substituted alkyl, or C(₁₋₆)unsubstituted alkyl, R⁹⁶ is C(₁₋₆)substituted alkyl, or C(₁₋₆)unsubstituted alkyl, and R⁹⁷ and R⁹⁸ are each independently H, C(₁₋₆)substituted alkyl, or C(₁₋₆)unsubstituted alkyl, or each pair: a) R⁷⁷ and R⁷⁸, b) R⁷⁹ and R⁸⁰, c) R⁸² and R⁸³, d) R⁸⁵ and R⁸⁶, e) R⁸⁸ and R⁸⁹, f) R⁹⁰ and R⁹¹, or g) R⁹² and R⁹³ are attached to adjacent ring-forming C atoms, and together with the ring-forming C atoms, form a substituted C₆ aryl ring or an unsubstituted C₆ aryl ring; R⁸¹, R⁸⁴ and R⁸⁷ each independently is C(₁₋₆)substituted alkyl, or C(₁₋₆)unsubstituted alkyl; and Y is CH₂, CHOH, CHO—CO—C(₁₋₆)unsubstituted alkyl, CHO—CO—C(₁₋₆)substituted alkyl, NCONH₂, N—C(₁₋₆)substituted alkyl, N—C(₁₋₆)unsubstituted alkyl, NH or N—C(O)OR⁹⁹, wherein R⁹⁹ is C(₁₋₆)unsubstituted alkyl, C(₁₋₆)substituted alkyl, C(₆₋₁₁)unsubstituted aralkyl or C(₆₋₁₁)substituted aralkyl; G¹⁰ is selected from the group consisting of: a straight C(₁₋₆)alkyl, a branched C(₃₋₆)alkyl and phenyl; G¹¹ is NHCH₂, NH, NHCO, SCH₂, O, or S; G¹² is H, NO₂, or OMe; G¹³ is H, NO₂, or OMe; each of G¹⁴, G^{14′} and G¹⁸ is independently NH, S, O, N—CH₃, N—CH₂—OCH₃, N—CH₂—COOH, N—CH₂—CH₂OH, N—CH₂—C(O)NH₂, CH—CH₃, N—R^{14′}, CH—R^{14′} or substituted C(₁₋₆)alkyl-NR⁵²R⁵³, wherein R^{14′} is C₍₁₋₆₎ substituted alkyl, C₍₁₋₆₎ unsubstituted alkyl,

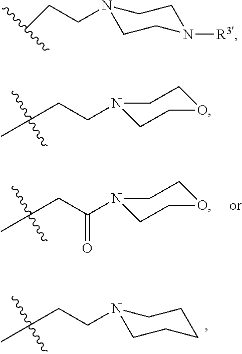

wherein R^{3′} is H, unsubstituted alkyl, or substituted alkyl, wherein the alkyl is 1-6 carbons in length, and the alkyl is optionally substituted with Br, F, Cl, I, OH, OMe, or N₃; each of G¹⁵, G^{15′} and G¹⁹ is independently N, CH or CG⁹; G¹⁶ is N or CH; G¹⁷ is N or CH; each of n, n², n³ and n⁴ is independently 0, 1, 2, 3, or 4; each Q¹ and Q¹⁴ is independently selected from the group consisting of: halogen, —OR²⁶, —O—(C₁₋₆)alkyl-NR²⁷R²⁸, —O—(C₁₋₆)alkyl-C(O)OR¹⁰⁰, —O—(C₁₋₆)alkyl-C(O)NHR¹⁰¹, —O—(C₁₋₆)alkyl-OC(O)R¹⁰², —O—(C₁₋₆)alkyl-OS(O)₂R¹⁰³, NO₂, NR¹⁰⁴R¹⁰⁵, —NHC(O)R¹⁰⁶, substituted C₍₁₋₆₎ alkyl, substituted C₍₁₋₆₎heteroalkyl, unsubstituted C₍₁₋₆₎alkyl, and unsubstituted C₍₁₋₆₎heteroalkyl; each Q² is independently selected from the group consisting of: halogen, —OR²⁹, —O—(C₁₋₆)alkyl-NR³⁰R³¹, —O—(C₁₋₆)alkyl-C(O)OR¹⁰⁷, —O—(C₁₋₆)alkyl-C(O)NHR¹⁰⁸, —O—(C₁₋₆)alkyl-OC(O)R¹⁰⁹, —O—(C₁₋₆)alkyl-OS(O)₂R¹¹⁰, NO₂, NR¹¹¹R¹¹², —NHC(O)R¹¹³, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q³ is independently selected from the group consisting of: halogen, —OR¹¹⁴, —O—(C₁₋₆)alkyl-NR¹¹⁵R¹¹⁶, —O—(C₁₋₆)alkyl-C(O)OR¹¹⁷, —O—(C₁₋₆)alkyl-C(O)NHR¹¹⁸, —O—(C₁₋₆)alkyl-OC(O)R¹¹⁹, —O—(C₁₋₆)alkyl-OS(O)₂R¹²⁰, NO₂, NR¹²¹R¹²², —NHC(O)R¹²³, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q⁴ is independently selected from the group consisting of: halogen, —OR³⁵, —O—(C₁₋₆)alkyl-NR³⁶R³⁷, —O—(C₁₋₆)alkyl-C(O)OR¹²⁴, —O—(C₁₋₆)alkyl-C(O)NHR¹²⁵, —O—(C₁₋₆)alkyl-OC(O)R¹²⁶, —O—(C₁₋₆)alkyl-OS(O)₂R¹²⁷, NO₂, NR¹²⁸R¹²⁹, —NHC(O)R¹³⁰, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q⁵ is independently selected from the group consisting of: halogen, —OR³⁸, —O—(C₁₋₆)alkyl-NR³⁹R⁴⁰, —O—(C₁₋₆)alkyl-C(O)OR¹³¹, —O—(C₁₋₆)alkyl-C(O)NHR¹³², —O—(C₁₋₆)alkyl-OC(O)R¹³³, —O—(C₁₋₆)alkyl-OS(O)₂R¹³⁴, NO₂, NR¹³⁵R¹³⁶, —NHC(O)R¹³⁷, substituted C(₁₋₆)alkyl, substituted C₍₁₋₆₎heteroalkyl, unsubstituted C₍₁₋₆₎alkyl, and unsubstituted C₍₁₋₆₎heteroalkyl; each Q⁶ is independently selected from the group consisting of: halogen, —OR⁴¹, —O—(C₁₋₆)alkyl-NR⁴²R⁴³, —O—(C₁₋₆)alkyl-C(O)OR¹³⁸, —O—(C₁₋₆)alkyl-C(O)NHR¹³⁹, —O—(C₁₋₆)alkyl-OC(O)R¹⁴⁰, —O—(C₁₋₆)alkyl-OS(O)₂R¹⁴¹, NO₂, NR¹⁴²R¹⁴³, —NHC(O)R¹⁴⁴, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q⁷ is independently selected from the group consisting of: halogen, —OR⁴⁴, —O—(C₁₋₆)alkyl-NR⁴⁵R⁴⁶, —O—(C₁₋₆)alkyl-C(O)OR¹⁴⁵, —O—(C₁₋₆)alkyl-C(O)NHR¹⁴⁶, —O—(C₁₋₆)alkyl-OC(O)R¹⁴⁷, —O—(C₁₋₆)alkyl-OS(O)₂R¹⁴⁸, NO₂, NR¹⁴⁹R¹⁵⁰, —NHC(O)R¹⁵¹, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q⁸ is independently selected from the group consisting of: halogen, —OR⁴⁷, —O—(C₁₋₆)alkyl-NR⁴⁸R⁴⁹, —O—(C₁₋₆)alkyl-C(O)OR¹⁵², —O—(C₁₋₆)alkyl-C(O)NHR¹⁵³, —O—(C₁₋₆)alkyl-OC(O)R¹⁵⁴, —O—(C₁₋₆)alkyl-OS(O)₂R¹⁵⁵, NO₂, NR¹⁵⁶R¹⁵⁷, —NHC(O)R¹⁵⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q⁹ is independently selected from the group consisting of: halogen, —OR¹⁵⁹, —O—(C₁₋₆)alkyl-NR¹⁶⁰R¹⁶¹, —O—(C₁₋₆)alkyl-C(O)OR¹⁶², —O—(C₁₋₆)alkyl-C(O)NHR¹⁶³, —O—(C₁₋₆)alkyl-OC(O)R¹⁶⁴, —O—(C₁₋₆)alkyl-OS(O)₂R¹⁶⁵, NO₂, NR¹⁶⁶R¹⁶⁷, —NHC(O)R¹⁶⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q¹⁰ is independently selected from the group consisting of: halogen, —OR¹⁶⁹, —O—(C₁₋₆)alkyl-NR¹⁷⁰R¹⁷¹, —O—(C₁₋₆)alkyl-C(O)OR¹⁷², —O—(C₁₋₆)alkyl-C(O)NHR¹⁷³, —O—(C₁₋₆)alkyl-OC(O)R¹⁷⁴, —O—(C₁₋₆)alkyl-OS(O)₂R¹⁷⁵, NO₂, NR¹⁷⁶R¹⁷⁷, —NHC(O)R¹⁷⁸, substituted C₍₁₋₆₎alkyl, substituted C₍₁₋₆₎heteroalkyl, unsubstituted C₍₁₋₆₎alkyl, and unsubstituted C(₁₋₆)heteroalkyl; each Q¹¹ is independently selected from the group consisting of: halogen, —OR¹⁷⁹, —O—(C₁₋₆)alkyl-NR¹⁸⁰R¹⁸¹, —O—(C₁₋₆)alkyl-C(O)OR¹⁸², —O—(C₁₋₆)alkyl-C(O)NHR¹⁸³, —O—(C₁₋₆)alkyl-OC(O)R¹⁸⁴, —O—(C₁₋₆)alkyl-OS(O)₂R¹⁸⁵, NO₂, NR¹⁸⁶R¹⁸⁷, —NHC(O)R¹⁸⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q¹² is independently selected from the group consisting of: halogen, —OR¹⁸⁹, —O—(C₁₋₆)alkyl-NR¹⁹⁰R¹⁹¹, —O—(C₁₋₆)alkyl-C(O)OR¹⁹², —O—(C₁₋₆)alkyl-C(O)NHR¹⁹³, —O—(C₁₋₆)alkyl-OC(O)R¹⁹⁴, —O—(C₁₋₆)alkyl-OS(O)₂R¹⁹⁵, NO₂, NR¹⁹⁶R¹⁹⁷, —NHC(O)R¹⁹⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q¹³ is independently selected from the group consisting of: halogen, —OR¹⁹⁹, —O—(C₁₋₆)alkyl-NR²⁰⁰R²⁰¹, —O—(C₁₋₆)alkyl-C(O)OR²⁰², —O—(C₁₋₆)alkyl-C(O)NHR²⁰³, —O—(C₁₋₆)alkyl-OC(O)R²⁰⁴, —O—(C₁₋₆)alkyl-OS(O)₂R²⁰⁵, NO₂, NR²⁰⁶R²⁰⁷, —NHC(O)R²⁰⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each R²⁶, R²⁷, R²⁸, R²⁹, R³⁰, R³¹, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R¹⁰⁰, R¹⁰⁴, R¹⁰⁵, R¹⁰⁷, R¹¹¹, R¹¹², R¹¹⁴, R¹¹⁵, R¹¹⁶, R¹¹⁷, R¹²¹, R¹²², R¹²⁴, R¹²⁸, R¹²⁹, R¹³¹, R¹³⁵, R¹³⁶, R¹³⁸, R¹⁴², R¹⁴³, R¹⁴⁵, R¹⁴⁹, R¹⁵⁰, R¹⁵², R¹⁵⁶, R¹⁵⁷, R¹⁵⁹, R¹⁶⁰, R¹⁶¹, R¹⁶², R¹⁶⁶, R¹⁶⁷, R¹⁶⁹, R¹⁷⁰, R¹⁷¹, R¹⁷², R¹⁷⁶, R¹⁷⁷, R¹⁷⁹, R¹⁸⁰, R¹⁸¹, R¹⁸², R¹⁸⁶, R¹⁸⁷, R¹⁸⁹, R¹⁹⁰, R¹⁹¹, R¹⁹², R¹⁹⁶, R¹⁹⁷, R¹⁹⁹, R²⁰⁰, R²⁰¹, R²⁰², R²⁰⁶ and R²⁰⁷ are independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and each pair: a) R²⁷ and R²⁸, b) R³⁰ and R³¹, c) R³⁶ and R³⁷, d) R³⁹ and R⁴⁰, e) R⁴² and R⁴³, f) R⁴⁵ and R⁴⁶, g) R⁴⁸ and R⁴⁹, h) R¹⁰⁴ and R¹⁰⁵, i) R¹¹¹ and R¹¹², j) R¹¹⁵ and R¹¹⁶, k) R¹²¹ and R¹²², l) R¹²⁸ and R¹²⁹, m) R¹³⁵ and R¹³⁶, n) R¹⁴² and R¹⁴³, o) R¹⁴⁹ and R¹⁵⁰, p) R¹⁵⁶ and R¹⁵⁷, q) R¹⁶⁰ and R¹⁶¹, r) R¹⁶⁶ and R¹⁶⁷, s) R¹⁷⁰ and R¹⁷¹, t) R¹⁷⁶ and R¹⁷⁷, u) R¹⁸⁰ and R¹⁸¹ v) R¹⁸⁶ and R¹⁸⁷, w) R¹⁹⁰ and R¹⁹¹, x) R¹⁹⁶ and R¹⁹⁷, y) R²⁰⁰ and R²⁰¹, and z) R²⁰⁶ and R²⁰⁷ may alternately be and independently as a pair be a 3-7 membered substituted heterocarbocyclic ring or a 3-7 membered unsubstituted heterocarbocyclic ring; R¹⁰¹, R¹⁰⁸, R¹¹⁸, R¹²⁵, R¹³², R¹³⁹, R¹⁴⁶, R¹⁵³, R¹⁶³, R¹⁷³, R¹⁸³, R¹⁹³ and R²⁰³ are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, unsubstituted C(₂₋₁₁)heteroaralkyl, substituted C(₁₋₆)alkyl-NR²⁰⁹R²¹⁰, unsubstituted C(₁₋₆)alkyl-NR²⁰⁹R²¹⁰, substituted C(₁₋₆)alkyl-N+R²¹¹R²¹²R²¹³, unsubstituted C(₁₋₆)alkyl-N⁺R²¹¹R²¹²R²¹³, substituted C(₁₋₆)alkyl-OR²¹⁴, unsubstituted C₍₁₋₆₎alkyl-OR²¹⁴,

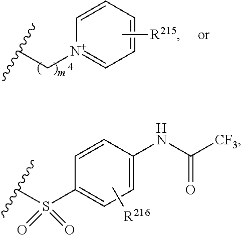

wherein m⁴ is 1, 2, 3, 4 or 5, R²⁰⁹, R²¹⁰, R²¹⁴, R²¹⁵ and R²¹⁶ are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C₍₁₋₆₎alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and R²⁰⁹ and R²¹⁰ may alternately be and independently as a pair be a 3-7 membered substituted heterocarbocyclic ring or a 3-7 membered unsubstituted heterocarbocyclic ring, and R²¹¹, R²¹² and R²¹³ are each independently unsubstituted C(₁₋₁₁)alkyl, or substituted C(₁₋₁₁)alkyl; and R¹⁰², R¹⁰³, R¹⁰⁶, R¹⁰⁹, R¹¹⁰, R¹¹³, R¹¹⁹, R¹²⁰, R¹²³, R¹²⁶, R¹²⁷, R¹³⁰, R¹³³, R¹³⁴, R¹³⁷, R¹⁴⁰, R¹⁴¹, R¹⁴⁴, R¹⁴⁷, R¹⁴⁸, R¹⁵¹, R¹⁵⁴, R¹⁵⁵, R¹⁵⁸, R¹⁶⁴, R¹⁶⁵, R¹⁶⁸, R¹⁷⁴, R¹⁷⁵, R¹⁷⁸, R¹⁸⁴, R¹⁸⁵, R¹⁸⁸, R¹⁹⁴, R¹⁹⁵, R¹⁹⁸, R²⁰⁴, R²⁰⁵ and R²⁰⁸ are each independently substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; (i) provided that G⁵ is absent only when G², G³ and G⁴ together form the ring moiety

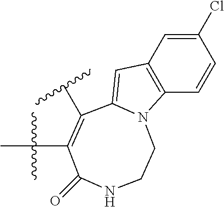 [SMILES: */C1=C(\*)c2cc3cc(Cl)ccc3n2CCNC1=O]

and G⁵ is absent when G², G³ and G⁴ together form the ring moiety

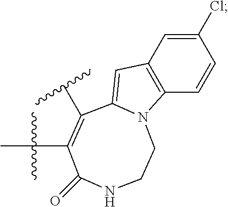 [SMILES: */C1=C(\*)c2cc3cc(Cl)ccc3n2CCNC1=O]

(ii) provided that when G³ is N, CH, or CG⁹ where G⁹ is C(O)OR and R⁹ is unsubstituted C₍₁₋₆₎ alkyl, G⁴ is other than

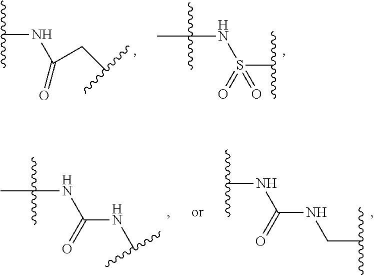 [SMILES: *CC(=O)N* ; *NS(*)(=O)=O ; *NC(=O)N* ; *CNC(=O)N*]

### and G⁵ is

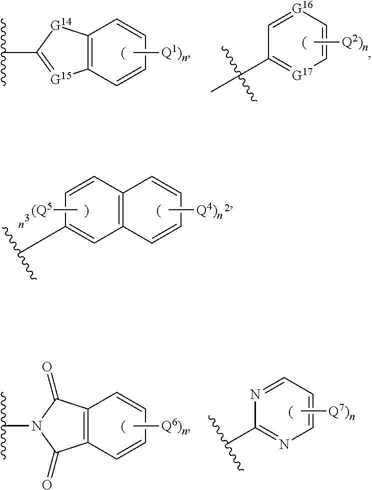

or a 5-membered heteroaryl optionally substituted with (Q⁸)ₙ and containing 1 or 2 heteroatoms each heteroatom independently selected from N, O and S, then n is at least 1 or n²+n³ is at least 1, and (a) when n is 1 or n²+n³=1, then Q¹, Q², Q⁴, Q⁵, Q⁶, Q⁷ or Q⁸ is independently selected from the group consisting of —OR^{26′}, —O—(C₁₋₆)alkyl-NR^{27′}R^{28′}, —O—(C₆)alkyl-COOR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NR^{104′}R^{105′}, and —NHC(O)R^{106′}, wherein R^{26′} is independently selected from the group consisting of substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₂₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; each R^{27′}, R^{28′}, R^{100′}, R^{104′} and R^{105′} is independently selected from the group consisting: H, substituted C₍₁₋₆₎alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C₍₁₋₆₎alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; or each pair: a) R^{27′} and R^{28′}, or b) R^{104′} and R^{105′} may alternately be and independently as a pair be a 3-7 membered substituted heterocarbocyclic ring or a 3-7 membered unsubstituted heterocarbocyclic ring; R^{101′} is H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, unsubstituted C(₂₋₁₁)heteroaralkyl, substituted C(₁₋₆)alkyl-NR^{209′}R^{210′}, unsubstituted C(₁₋₆)alkyl-NR^{209′} R²¹⁰, substituted C(₁₋₆)alkyl-N⁺R^{211′}R^{212′}R^{213′}, unsubstituted C(₁₋₆)alkyl-N⁺R^{211′}R^{212′}R^{213′}, substituted C(₁₋₆)alkyl-OR^{214′}, unsubstituted C(₁₋₆)alkyl-OR^{214′},

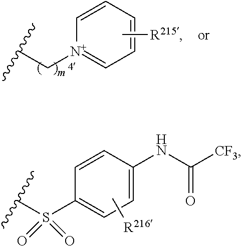

wherein m is 1, 2, 3, 4 or 5, R^{209′}R^{210′} R^{214′} R and R²¹⁶ are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and R^{209′} and R^{210′} may alternately be and independently as a pair be a 3-7 membered substituted heterocarbocyclic ring or a 3-7 membered unsubstituted heterocarbocyclic ring, and R^{211′}, R^{212′} and R^{213′} are each independently unsubstituted C(₁₋₁₁)alkyl, or substituted C(₁₋₁₁)alkyl; and R^{102′}, R^{103′}, and R^{106′} are each independently substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and (b) when n is at least 2 or n²+n³ is at least 2, then a first Q¹, Q², Q⁴, Q⁵, Q⁶, Q⁷ or Q⁸ is independently selected from the group consisting of —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NR^{104′}R^{105′} and —NHC(O)R^{106′}, wherein each of R^{26′}, R^{27′}R^{28′}, R^{100′}, R^{101′}, R^{102′}, R^{103′}, R^{104′}, R^{105′}, and R^{106′} is as defined above; and the remaining Q¹, Q², Q⁴, Q⁵, Q⁶, Q⁷ or Q⁸ are each independently selected from the group consisting of halogen, —OR^{26′}, —O—(C₁₋₆)alkyl-NR²⁷R^{28′}, —O—(C₁₋₆)alkyl-C(O)OR^{100′}, —O—(C₁₋₆)alkyl-C(O)NHR¹⁰¹, —O—(C₁₋₆)alkyl-OC(O)R¹⁰², —O—(C₁₋₆)alkyl-OS(O)₂R^{103′}, NO₂, NR^{104′}R^{105′}, —NHC(O)R^{106′}, substituted C(₁₋₆)alkyl, substituted C₍₁₋₆₎heteroalkyl, unsubstituted C₍₁₋₆₎alkyl, and unsubstituted C₍₁₋₆₎heteroalkyl; wherein each R^{26′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and each of R^{27′}, R^{28′}, R^{100′}, R^{101′}, R^{102′}, R^{103′}, R^{104′}, R^{105′}, and R^{106′} is as defined above; and (iii) provided that when G³ is N, CH, or CG⁹ where G⁹ is C(O)OR⁹ and R⁹ is unsubstituted C₍₁₋₆₎ alkyl, G⁴ is other than

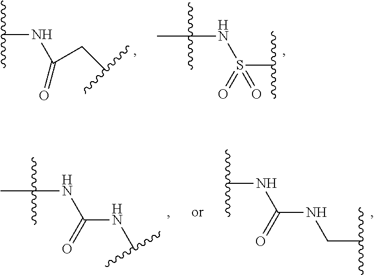 [SMILES: *CC(=O)N* ; *NS(*)(=O)=O ; *NC(=O)N* ; *CNC(=O)N*]

### and G⁵ is

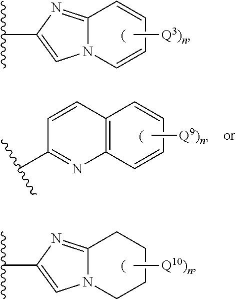

then n is at least 1 wherein each of Q³, Q⁹ and Q¹⁰ is as defined above, and wherein the compound, or salt thereof, has anti-bacterial activity.

Illustrative embodiments of the present invention provide a compound having a structure of formula (1):

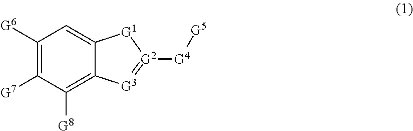

or a salt thereof, wherein: G¹ is NH, O, or S; G², G³ and G⁴ may either: i) together form a ring moiety selected from the group consisting of:

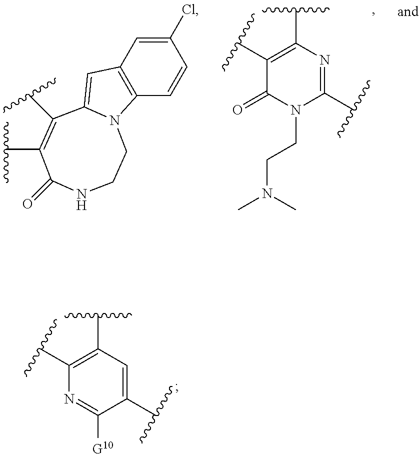

or ii) together do not form a ring moiety wherein G² is C; G³ is N, CH or CG⁹; and G⁴ is selected from the group consisting of: a bond,

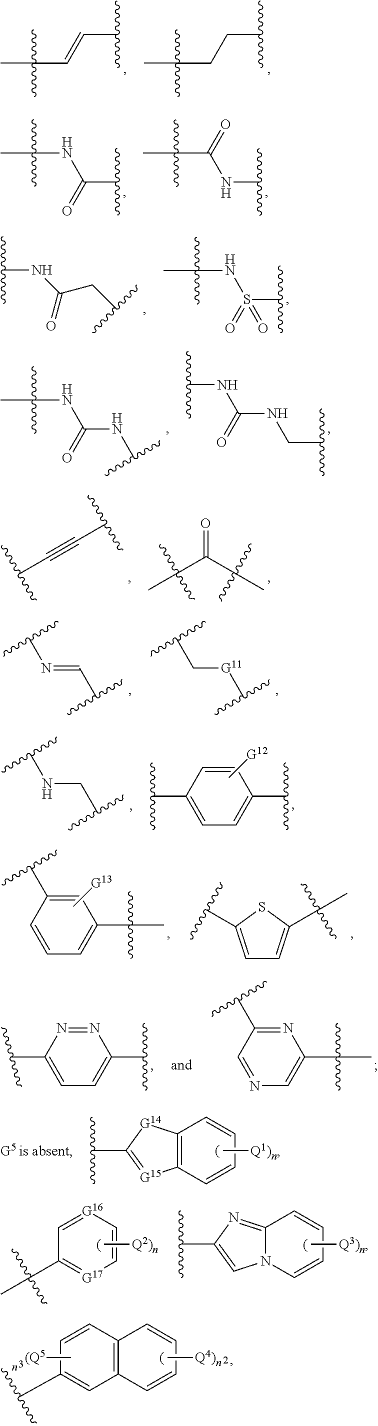

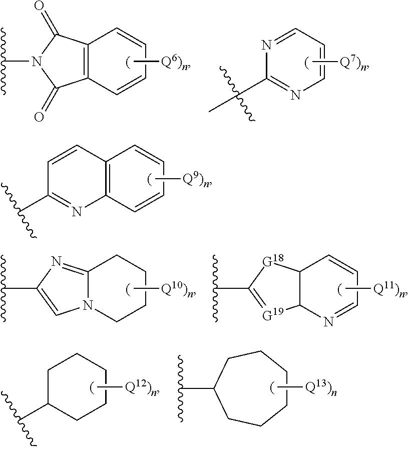

or a 5-membered heteroaryl optionally substituted with (Q⁸)ₙ and containing 1 or 2 heteroatoms each heteroatom independently selected from N, O and S; G⁶ is H, halogen, CF₃, NO₂, substituted (C₁₋₁₁)alkyl, unsubstituted (C₁₋₁₁)alkyl, substituted (C₁₋₁₁)alkoxyl, unsubstituted (C₁₋₁₁) alkoxyl, substituted (C₆₋₁₁)aryloxy, unsubstituted (C₆₋₁₁)aryloxy, C(O)OR⁵⁰, or

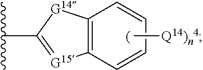

G⁷ is H, halogen, CF₃, NO₂, substituted (C₁₋₁₁)alkyl, unsubstituted (C₁₋₁₁)alkyl, substituted (C₁₋₁₁) alkoxyl, unsubstituted (C₁₋₁₁) alkoxy, substituted (C₆₋₁₁)aryloxy, unsubstituted (C₆₋₁₁)aryloxy, C(O)OR⁵¹, or

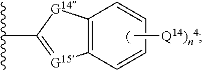

R⁵⁰ and R⁵¹ are each independently substituted (C₁₋₆)alkyl, unsubstituted (C₁₋₆)alkyl, substituted (C₁₋₆)heteroalkyl or unsubstituted (C₁₋₆) heteroalkyl; G⁸ is H, C(═O)N(CH₃)₂, or C(═O)N(H)C(H₂)C₆H₅; G⁹ is CF₃, —SO₂NH₂, —NH₂, —C(CF₃)₂OH, —C(CF₃)(H)OH, —C(CF₃)(CH₃)OH, —C(NOH)C(R²¹)(R²²)(R²³), C(NOH)N(R²⁴)(R²⁵), C(NOR⁶⁰)C(R⁶¹)(R⁶²)(R⁶³), substituted (C₁₋₆) alkyl-NR⁶⁴R⁶⁵ unsubstituted (C₁₋₆) alkyl-NR⁶⁴R⁶⁵, substituted (C₆₋₁₁) aryl, unsubstituted (C₁₀)aryl, substituted (C₁₋₁₁) heteroaryl, unsubstituted (C₁₋₁₁) heteroaryl, substituted (C₆₋₁₁) arylcarbonyl, unsubstituted (C₆₋₁₁) arylcarbonyl, substituted (C₁₋₁₁) heteroarylcarbonyl, unsubstituted (C₁₋₁₁) heteroarylcarbonyl, —CO-substituted-carbocycle, —CO-unsubstituted-carbocycle, —CO-substituted-heterocarbocycle, —CO-unsubstituted-heterocarbocycle, —CO-substituted-C(₁₋₆)alkyl-OR¹, —CO-unsubstituted-C(₁ 6)alkyl-OR¹, —CO-substituted-C(₁₋₆)alkyl-NR²R³, —CO-unsubstituted-C(₁₋₆)alkyl-NR²R³, —CO-substituted-C(₁₋₆)alkyl-C(O)OR⁴, —CO-unsubstituted-C(₁₋₆)alkyl-C(O)OR⁴; —CO-substituted-C(₁₋₆)alkyl-C(O)NR⁵R⁶, —CO-unsubstituted-C(₁₋₆)alkyl-C(O)NR⁵R⁶, —C(O)NR⁷R⁸, —C(O)OR⁹, —C(O)C(O)OR¹², —C(O)C(O)NR¹³R¹⁴, —NR¹⁵R¹⁶, —N(H)C(O)substituted-C(₁₋₆)alkyl, —N(H)C(O)unsubstituted-C(₁₋₆)alkyl, —N(H)C(O)substituted-C(₁₋₆)haloalkyl, —N(H)C(O)unsubstituted-C(₁₋₆)haloalkyl, —N(H)C(O)substituted-C(₆₋₁₁)aryl, —N(H)C(O)unsubstituted-C(₆₋₁₁)aryl, —N(H)C(O)substituted-C(₁₋₁)heteroaryl, —N(H)C(O)unsubstituted-C(₁₋₁₁)heteroaryl, —N(H)C(O)NR¹⁷R¹⁸, —N(H)CO-substituted-C(₁₋₆)alkyl-OR¹⁹, —N(H)CO-unsubstituted-C(₁₋₆)alkyl-OR¹⁹, each of R¹, R², R³, R⁴, R⁵, R⁶, R¹², R¹³, R¹⁴, R¹⁷, R¹⁸, R¹⁹, R²⁴, and R²⁵ is independently selected from the group consisting of: H, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl, and each of R²¹, R²², R²³, R⁶¹, R⁶² and R⁶³ is independently selected from the group consisting of: H, F, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl; each pair: a) R² and R³, b) R⁵ and R⁶, c) R¹³ and R¹⁴, and d) R¹⁷ and R¹⁸ may alternately be and independently as a pair be a 3-7 membered substituted heterocarbocyclic ring or a 3-7 membered unsubstituted heterocarbocyclic ring; R⁶⁰ is unsubstituted C(₁₋₁₁)alkyl, substituted C(₁₋₁₁)alkyl, unsubstituted C(₁₋₁₁)alkyl-NR⁶⁶R⁶⁷, substituted C(₁₋₁₁)alkyl-NR⁶⁶R⁶⁷, unsubstituted C(₁₋₁₁)alkyl-N⁺R⁶⁸R⁶⁹R⁷⁰, or substituted C(₁₋₁₁)alkyl-N⁺R⁶⁸R⁶⁹R⁷⁰, wherein R⁶⁶ and R⁶⁷ are each independently H, unsubstituted C(₁₋₁₁)alkyl or substituted C(₁₋₁₁)alkyl, and R⁶⁸, R⁶⁹ and R⁷⁰ are each independently unsubstituted C(₁₋₁₁)alkyl, or substituted C(₁₋₁₁)alkyl, each of R¹⁵ and R¹⁶ is independently selected from the group consisting of: H, substituted C(₁₋₆)alkyl, substituted C(₁₋₁₁)aryl, substituted C(₁₋₁₁)heteroaryl, substituted C(₇₋₁₁)aralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl, or R¹⁵ and R¹⁶ may alternately be a 3-7 membered unsubstituted heterocarbocyclic ring; each of R⁶⁴ and R⁶⁵ is independently selected from the group consisting of: H, substituted C(₃₋₆)alkyl, substituted C(₁₋₁₁)aryl, substituted C(₁₋₁₁)heteroaryl, substituted C(₇₋₁₁)aralkyl, unsubstituted C(₂₋₁₁)alkyl, unsubstituted C(₁₋₁₁)aryl, unsubstituted C(₁₋₁₁)heteroaryl, and unsubstituted C(₈₋₁₁)aralky, or R⁶⁴ and R⁶⁵ may alternately be a 3-7 membered substituted heterocarbocyclic ring or a 3-7 membered unsubstituted heterocarbocyclic ring; each of R⁷ and R⁸ are either I) independently selected from the group consisting of: H, substituted C(₁₋₆)alkyl, substituted C(₁₋₆)alkyl-NR⁵²R⁵³, unsubstituted C(₁₋₆)alkyl-NR⁵²R⁵³, substituted C(₁₋₆)alkyl-N⁺R⁷¹R⁷²R⁷³, unsubstituted C(₁₋₆)alkyl-N⁺R⁷¹R⁷²R⁷³, substituted C(₁₋₆)alkyl-OC(O)unsubstituted C(₁₋₆)alkyl-NR⁷⁴R⁷⁵, unsubstituted C(₁₋₆)alkyl-OC(O)unsubstituted C(₁₋₆)alkyl-NR⁷⁴R⁷⁵, substituted C(₁₋₆)alkyl-C(O)NHS(O)₂R⁷⁶, unsubstituted C(₁₋₆)alkyl-C(O)NHS(O)₂R⁷⁶, substituted C(₆₋₁₁)aryl, substituted C(₃₋₁₁)carbocyclic, substituted C(₄₋₇)heterocarbocycle, substituted C(₄₋₇)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₃₋₁₁)carbocyclic, unsubstituted C(₁₋₁₁)heterocarbocycle, unsubstituted C(₁₋₁₁)heteroaryl, unsubstituted C(₇₋₁₁)aralkyl, and unsubstituted C(₂₋₁₁)heteroaralkyl wherein each of R⁵², R⁵³, R⁷⁴ and R⁷⁵ is selected from the group consisting of: H, unsubstituted C(₁₋₆)alkyl, substituted C(₃₋₇)heterocycloalkyl, unsubstituted C(₃₋₇)heterocycloalkyl, substituted C(₁₋₆)alkyl, substituted C(₃₋₇)cycloalkyl and unsubstituted C(₃₋₇)cycloalkyl, or each pair: a) R⁵² and R⁵³, or (b) R⁷⁴ and R⁷⁵, together form a 3-7 membered substituted heterocarbocyclic ring or a 3-7 membered unsubstituted heterocarbocyclic ring, and wherein each of R⁷¹, R⁷², R⁷³ and R⁷⁶ is independently unsubstituted C(₁₋₁₁)alkyl, or substituted C(₁₋₁₁)alkyl, or II) together form a 3-7 membered substituted heterocarbocyclic ring or a 3-7 membered unsubstituted heterocarbocyclic ring; R⁹ is selected from the group consisting of substituted C(₁₋₆)alkyl, substituted C(₁₋₆)alkyl-NR¹⁰R¹¹, unsubstituted C(₁₋₆)alkyl-NR¹⁰R¹¹, substituted C(₁₋₆)alkyl-OR²⁰, unsubstituted C(₁₋₆)alkyl-OR²⁰, and unsubstituted C(₄₋₆)alkyl wherein each of R¹⁰, R¹¹ and R²⁰ is independently selected from the group consisting of: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; R¹⁰ and R¹¹ may alternately as a pair be a 3-7 membered substituted heterocarbocyclic ring or a 3-7 membered unsubstituted heterocarbocyclic ring, or G⁹ is

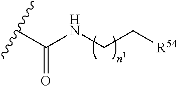

wherein n¹ is 1, 2, 3 or 4 and R⁵⁴ is

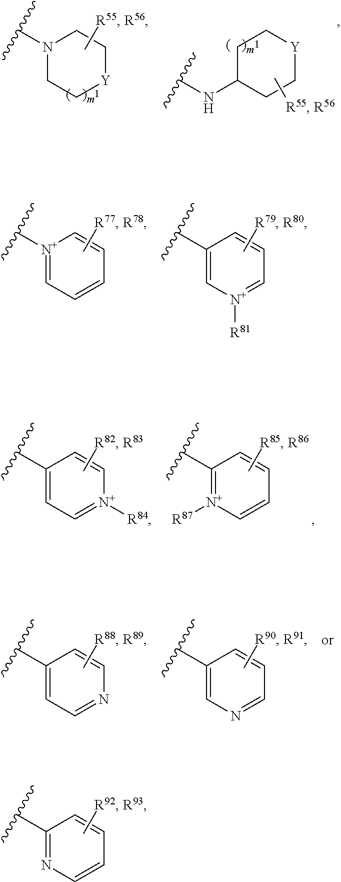

wherein m¹=0, 1 or 2, R⁵⁵ and R⁵⁶ are independently H, carbonyl (═O), Me, Ph, CO₂R⁹⁴, CO₂NH₂, C(₁₋₆)substituted alkyl or C(₁₋₆)unsubstituted alkyl, wherein R⁹⁴ is H, C(₁₋₆)unsubstituted alkyl or C(₁₋₆)substituted alkyl; R⁷⁷, R⁷⁸, R⁷⁹, R⁸⁰, R⁸², R⁸³, R⁸⁵, R⁸⁶, R⁸⁸, R⁸⁹, R⁹⁰, R⁹¹, R⁹² and R⁹³ are each independently H, C(₁₋₆)substituted alkyl, C(₁₋₆)unsubstituted alkyl, substituted C(₁₋₆)heteroalkyl, unsubstituted C₍₁₋₆₎ heteroalkyl, OR⁹⁵, C(O)R⁹⁶, or NR⁹⁷R⁹⁸, wherein R⁹⁵ is H, C(₁₋₆)substituted alkyl, or C(₁₋₆)unsubstituted alkyl, R⁹⁶ is C(₁₋₆)substituted alkyl, or C(₁₋₆)unsubstituted alkyl, and R⁹⁷ and R⁹⁸ are each independently H, C(₁₋₆)substituted alkyl, or C(₁₋₆)unsubstituted alkyl, or each pair: a) R⁷⁷ and R⁷⁸, b) R⁷⁹ and R⁸⁰, c) R⁸² and R⁸³, d) R⁸⁵ and R⁸⁶, e) R⁸⁸ and R⁸⁹, f) R⁹⁰ and R⁹¹, or g) R⁹² and R⁹³ are attached to adjacent ring-forming C atoms, and together with the ring-forming C atoms, form a substituted C₆ aryl ring or an unsubstituted C₆ aryl ring; R⁸¹, R⁸⁴ and R⁸⁷ each independently is C(₁₋₆)substituted alkyl, or C(₁₋₆)unsubstituted alkyl; and Y is CH₂, CHOH, CHO—CO—C(₁₋₆)unsubstituted alkyl, CHO—CO—C(₁₋₆)substituted alkyl, NCONH₂, N—C(₁₋₆)substituted alkyl, N—C(₁₋₆)unsubstituted alkyl, NH or N—C(O)OR⁹⁹, wherein R⁹⁹ is C(₁₋₆)unsubstituted alkyl, C(₁₋₆)substituted alkyl, C(₆₋₁₁)unsubstituted aralkyl or C(₆₋₁₁)substituted aralkyl; G¹⁰ is selected from the group consisting of: a straight C(₁₋₆)alkyl, a branched C(₃₋₆)alkyl and phenyl; G¹¹ is NHCH₂, NH, NHCO, SCH₂, O, or S; G¹² is H, NO₂, or OMe; G¹³ is H, NO₂, or OMe; each of G¹⁴, G^{14′} and G¹⁸ is independently NH, S, O, N—CH₃, N—CH₂—OCH₃, N—CH₂—COOH, N—CH₂—CH₂OH, N—CH₂—C(O)NH₂, CH—CH₃, N—R^{14′}, CH—R^{14′} or substituted C(₁₋₆)alkyl-NR⁵²R⁵³, wherein R^{14′} is C₍₁₋₆₎ substituted alkyl, C₍₁₋₆₎ unsubstituted alkyl,

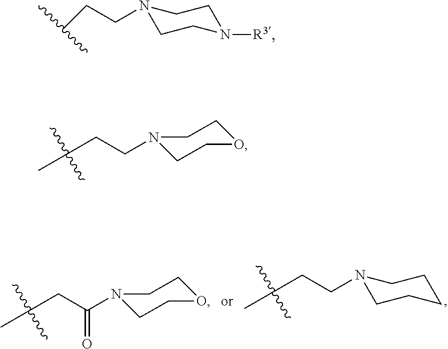

wherein R^{3′} is H, unsubstituted alkyl, or substituted alkyl, wherein the alkyl is 1-6 carbons in length, and the alkyl is optionally substituted with Br, F, Cl, I, OH, OMe, or N₃; each of G¹⁵, G^{15′} and G¹⁹ is independently N, CH or CG⁹; G¹⁶ is N or CH; G¹⁷ is N or CH; each of n, n², n³ and n⁴ is independently 0, 1, 2, 3 or 4; each Q¹ and Q¹⁴ is independently selected from the group consisting of: halogen, —OR²⁶, —O—(C₁₋₆)alkyl-NR²⁷R²⁸, —O—(C₁₋₆)alkyl-C(O)OR¹⁰⁰, —O—(C₁₋₆)alkyl-C(O)NHR¹⁰¹, —O—(C₁₋₆)alkyl-OC(O)R¹⁰², —O—(C₁₋₆)alkyl-OS(O)₂R¹⁰³, NO₂, NR¹⁰⁴R¹⁰⁵, —NHC(O)R¹⁰⁶, substituted C(₁₋₆)alkyl, substituted C(₁₋₆)heteroalkyl, unsubstituted C(₁₋₆)alkyl, and unsubstituted C(₁₋₆)heteroalkyl; each Q² is independently selected from the group consisting of: halogen, —OR²⁹, —O—(C₁₋₆)alkyl-NR³⁰R³¹, —O—(C₁₋₆)alkyl-C(O)OR¹⁰⁷, —O—(C₁₋₆)alkyl-C(O)NHR¹⁰⁸, —O—(C₁₋₆)alkyl-OC(O)R¹⁰⁹, —O—(C₁₋₆)alkyl-OS(O)₂R¹¹⁰, NO₂, NR¹¹¹R¹¹², —NHC(O)R¹¹³, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q³ is independently selected from the group consisting of: halogen, —OR¹¹⁴, —O—(C₁₋₆)alkyl-NR¹¹⁵R¹¹⁶, —O—(C₁₋₆)alkyl-C(O)OR¹¹⁷, —O—(C₁₋₆)alkyl-C(O)NHR¹¹⁸, —O—(C₁₋₆)alkyl-OC(O)R¹¹⁹, —O—(C₁₋₆)alkyl-OS(O)₂R¹²⁰, NO₂, NR¹²¹R¹²², —NHC(O)R¹²³, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q⁴ is independently selected from the group consisting of: halogen, —OR³⁵, —O—(C₁₋₆)alkyl-NR³⁶R³⁷, —O—(C₁₋₆)alkyl-C(O)OR¹²⁴, —O—(C₁₋₆)alkyl-C(O)NHR¹²⁵, —O—(C₁₋₆)alkyl-OC(O)R¹²⁶, —O—(C₁₋₆)alkyl-OS(O)₂R¹²⁷, NO₂, NR¹²⁸R¹²⁹, —NHC(O)R¹³⁰, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q⁵ is independently selected from the group consisting of: halogen, —OR³⁸, —O—(C₁₋₆)alkyl-NR³⁹R⁴⁰, —O—(C₁₋₆)alkyl-C(O)OR¹³¹, —O—(C₁₋₆)alkyl-C(O)NHR¹³², —O—(C₁₋₆)alkyl-OC(O)R¹³³, —O—(C₁₋₆)alkyl-OS(O)₂R¹³⁴, NO₂, NR¹³⁵R¹³⁶, —NHC(O)R¹³⁷, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q⁶ is independently selected from the group consisting of: halogen, —OR⁴¹, —O—(C₁₋₆)alkyl-NR⁴²R⁴³, —O—(C₁₋₆)alkyl-C(O)OR¹³⁸, —O—(C₁₋₆)alkyl-C(O)NHR¹³⁹, —O—(C₁₋₆)alkyl-OC(O)R¹⁴⁰, —O—(C₁₋₆)alkyl-OS(O)₂R¹⁴¹, NO₂, NR¹⁴²R¹⁴³, —NHC(O)R¹⁴⁴, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q⁷ is independently selected from the group consisting of: halogen, —OR⁴⁴, —O—(C₁₋₆)alkyl-NR⁴⁵R⁴⁶, —O—(C₁₋₆)alkyl-C(O)OR¹⁴⁵, —O—(C₁₋₆)alkyl-C(O)NHR¹⁴⁶, —O—(C₁₋₆)alkyl-OC(O)R¹⁴⁷, —O—(C₁₋₆)alkyl-OS(O)₂R¹⁴⁸, NO₂, NR¹⁴⁹R¹⁵⁰, —NHC(O)R¹⁵¹, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q⁸ is independently selected from the group consisting of: halogen, —OR⁴⁷, —O—(C₁₋₆)alkyl-NR⁴⁸R⁴⁹, —O—(C₁₋₆)alkyl-C(O)OR¹⁵², —O—(C₁₋₆)alkyl-C(O)NHR¹⁵³, —O—(C₁₋₆)alkyl-OC(O)R¹⁵⁴, —O—(C₁₋₆)alkyl-OS(O)₂R¹⁵⁵, NO₂, NR¹⁵⁶R¹⁵⁷, —NHC(O)R¹⁵⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q⁹ is independently selected from the group consisting of: halogen, —OR¹⁵⁹, —O—(C₁₋₆)alkyl-NR¹⁶⁰R¹⁶¹, —O—(C₁₋₆)alkyl-C(O)OR¹⁶², —O—(C₁₋₆)alkyl-C(O)NHR¹⁶³, —O—(C₁₋₆)alkyl-OC(O)R¹⁴, —O—(C₁₋₆)alkyl-OS(O)₂R¹⁶⁵, NO₂, NR¹⁶⁶R¹⁶⁷, —NHC(O)R¹⁶⁸, substituted C(₁₋₆)alkyl, substituted C(₁₋₆)heteroalkyl, unsubstituted C(₁₋₆)alkyl, and unsubstituted C(₁₋₆)heteroalkyl; each Q¹⁰ is independently selected from the group consisting of: halogen, —OR¹⁶⁹, —O—(C₁₋₆)alkyl-NR¹⁷⁰R¹⁷¹, —O—(C₁₋₆)alkyl-C(O)OR¹⁷², —O—(C₁₋₆)alkyl-C(O)NHR¹⁷³, —O—(C₁₋₆)alkyl-OC(O)R¹⁷⁴, —O—(C₁₋₆)alkyl-OS(O)₂R¹⁷⁵, NO₂, NR¹⁷⁶R¹⁷⁷, —NHC(O)R¹⁷⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q¹¹ is independently selected from the group consisting of: halogen, —OR¹⁷⁹, —O—(C₁₋₆)alkyl-NR¹⁸⁰R¹⁸¹, —O—(C₁₋₆)alkyl-C(O)OR¹⁸², —O—(C₁₋₆)alkyl-C(O)NHR¹⁸³, —O—(C₁₋₆)alkyl-OC(O)R¹⁸⁴, —O—(C₁₋₆)alkyl-OS(O)₂R¹⁸⁵, NO₂, NR¹⁸⁶R¹⁸⁷, —NHC(O)R¹⁸⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q¹² is independently selected from the group consisting of: halogen, —OR¹⁸⁹, —O—(C₁₋₆)alkyl-NR¹⁹⁰R¹⁹¹, —O—(C₁₋₆)alkyl-C(O)OR¹⁹², —O—(C₁₋₆)alkyl-C(O)NHR¹⁹³, —O—(C₁₋₆)alkyl-OC(O)R¹⁹⁴, —O—(C₁₋₆)alkyl-OS(O)₂R¹⁹⁵, NO₂, NR¹⁹⁶R¹⁹⁷, —NHC(O)R¹⁹⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each Q¹³ is independently selected from the group consisting of: halogen, —OR¹⁹⁹, —O—(C₁₋₆)alkyl-NR²⁰R²⁰¹, —O—(C₁₋₆)alkyl-C(O)OR²⁰², —O—(C₁₋₆)alkyl-C(O)NHR²⁰³, —O—(C₁₋₆)alkyl-OC(O)R²⁰⁴, —O—(C₁₋₆)alkyl-OS(O)₂R²⁰⁵, NO₂, NR²⁰⁶R²⁰⁷, —NHC(O)R²⁰⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; each R²⁶, R²⁷, R²⁸, R²⁹, R³⁰, R³¹, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R¹⁰⁰, R¹⁰⁴, R¹⁰⁵, R¹⁰⁷, R¹¹¹, R¹¹², R¹¹⁴, R¹¹⁵, R¹¹⁶, R¹¹⁷, R¹²¹, R¹²², R¹²⁴, R¹²⁸, R¹²⁹, R¹³¹, R¹³⁵, R¹³⁶, R¹³⁸, R¹⁴², R¹⁴³, R¹⁴⁵, R¹⁴⁹, R¹⁵⁰, R¹⁵², R¹⁵⁶, R¹⁵⁷, R¹⁵⁹, R¹⁶⁰, R¹⁶¹, R¹⁶², R¹⁶⁶, R¹⁶⁷, R¹⁶⁹, R¹⁷⁰, R¹⁷¹, R¹⁷², R¹⁷⁶, R¹⁷⁷, R¹⁷⁹, R¹⁸⁰, R¹⁸¹, R¹⁸², R¹⁸⁶, R¹⁸⁷, R¹⁸⁹, R¹⁹⁰, R¹⁹¹, R¹⁹², R¹⁹⁶, R¹⁹⁷, R¹⁹⁹, R²⁰⁰, R²⁰¹, R²⁰², R²⁰⁶ and R²⁰⁷ are independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and each pair: a) R²⁷ and R²⁸, b) R³⁰ and R³¹, c) R³⁶ and R³⁷, d) R³⁹ and R⁴⁰, e) R⁴² and R⁴³, f) R⁴⁵ and R⁴⁶, g) R⁴⁸ and R⁴⁹, h) R¹⁰⁴ and R¹⁰⁵, i) R¹¹¹ and R¹¹², j) R¹¹⁵ and R¹¹⁶, k) R¹²¹ and R¹²², l) R¹²⁸ and R¹²⁹, m) R¹³⁵ and R¹³⁶, n) R¹⁴² and R¹⁴³, o) R¹⁴⁹ and R¹⁵⁰, p) R¹⁵⁶ and R¹⁵⁷, q) R¹⁶⁰ and R¹⁶¹, r) R¹⁶⁶ and R¹⁶⁷, s) R¹⁷⁰ and R¹⁷¹, t) R¹⁷⁶ and R¹⁷⁷, u) R¹⁸⁰ and R¹⁸¹, v) R¹⁸⁶ and R¹⁸⁷, w) R¹⁹⁰ and R¹⁹¹, x) R¹⁹⁶ and R¹⁹⁷, y) R²⁰⁰ and R²⁰¹, and z) R²⁰⁶ and R²⁰⁷ may alternately be and independently as a pair be a 3-7 membered substituted heterocarbocyclic ring or a 3-7 membered unsubstituted heterocarbocyclic ring; R¹⁰¹, R¹⁰⁸, R¹¹⁸, R¹²⁵, R¹³², R¹³⁹, R¹⁴⁶, R¹⁵³, R¹⁶³, R¹⁷³, R¹⁸³, R¹⁹³ and R²⁰³ are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, unsubstituted C(₂₋₁₁)heteroaralkyl, substituted C(₁₋₆)alkyl-NR²⁰⁹R²¹⁰, unsubstituted C(₁₋₆)alkyl-NR²⁰⁹R²¹⁰, substituted C(₁₋₆)alkyl-N⁺R²¹¹R²¹²R²¹³, unsubstituted C(₁₋₆)alkyl-N⁺R²¹¹R²¹²R²¹³, substituted C(₁₋₆)alkyl-OR²¹⁴, unsubstituted C(₁₋₆)alkyl-OR²¹⁴,

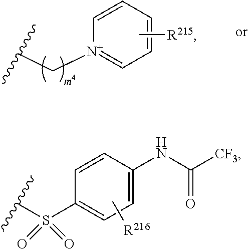

wherein m is 1, 2, 3, 4 or 5, R²⁰⁹, R²¹⁰, R²¹⁴, R²¹⁵ and R²¹⁶ are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and R²⁰⁹ and R²¹⁰ may alternately be and independently as a pair be a 3-7 membered substituted heterocarbocyclic ring or a 3-7 membered unsubstituted heterocarbocyclic ring, and R²¹¹, R²¹² and R²¹³ are each independently unsubstituted C(₁₋₁₁)alkyl, or substituted C(₁₋₁₁)alkyl; and R¹⁰², R¹⁰³, R¹⁰⁶, R¹⁰⁹, R¹¹⁰, R¹¹³, R¹¹⁹, R¹²⁰, R¹²³, R¹²⁶, R¹²⁷, R¹³⁰, R¹³³, R¹³⁴, R¹³⁷, R¹⁴⁰, R¹⁴¹, R¹⁴⁴, R¹⁴⁷, R¹⁴⁸, R¹⁵¹, R¹⁵⁴, R¹⁵⁵, R¹⁵⁸, R¹⁶⁴, R¹⁶⁵, R¹⁶⁸, R¹⁷⁴, R¹⁷⁵, R¹⁷⁸, R¹⁸⁴, R¹⁸⁵, R¹⁸⁸, R¹⁹⁴, R¹⁹⁵, R¹⁹⁸, R²⁰⁴, R²⁰⁵ and R²⁰⁸ are each independently substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; (i) provided that G⁵ is absent only when G², G³ and G⁴ together form the ring moiety

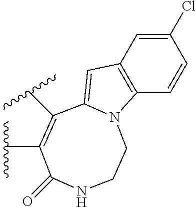 [SMILES: */C1=C(\*)c2cc3cc(Cl)ccc3n2CCNC1=O]

and G⁵ is absent when G², G³ and G⁴ together form the ring moiety

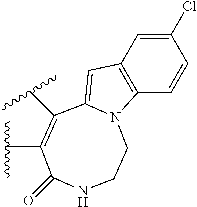 [SMILES: */C1=C(\*)c2cc3cc(Cl)ccc3n2CCNC1=O]

(ii) provided that when G³ is N, CH, or CG⁹ where G⁹ is C(O)OR⁹ and R⁹ is unsubstituted C(₄₋₆) alkyl, G⁴ is other than

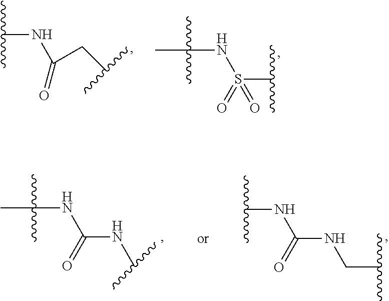

### and G⁵ is

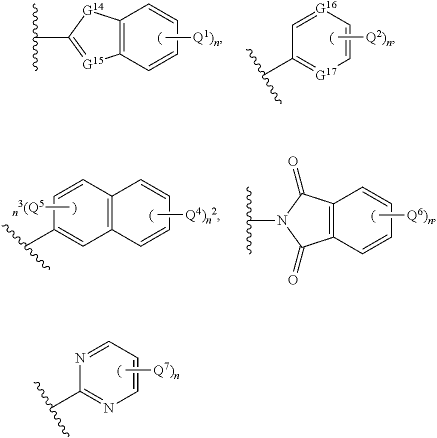

or a 5-membered heteroaryl optionally substituted with (Q⁸)ₙ and containing 1 or 2 heteroatoms each heteroatom independently selected from N, O and S, then n is at least 1 or n²+n³ is at least 1, and (a) when n is 1 or n²+n³=1, then Q¹, Q², Q⁴, Q⁵, Q⁶, Q⁷ or Q⁸ is independently selected from the group consisting of —OR^{26′}, —O—(C₁₋₆)alkyl-NR^{27′}R^{28′}, —′O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, and —NHC(O)R^{106′}, wherein R^{26′} is independently selected from the group consisting of substituted C(₁₋₆)alkyl, substituted C(₇₋₁₁)aralkyl, substituted C(₂₋₁₁)heteroaralkyl, unsubstituted C(₅₋₁₁)alkyl, unsubstituted C(₇₋₁₁)aralkyl, and unsubstituted C(₂₋₁₁)heteroaralkyl; each of R^{27′}, R^{28′}, and R^{100′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; or R^{27′} and R^{28′} may alternately as a pair be a 3-7 membered substituted heterocarbocyclic ring or a 3-7 membered unsubstituted heterocarbocyclic ring; R^{101′} is H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, unsubstituted C(₂₋₁₁)heteroaralkyl, substituted C(₁₋₆)alkyl-NR^{209′}R^{210′}, unsubstituted C(₁₋₆)alkyl-NR^{209′}R^{210′}, substituted C(₁₋₆)alkyl-NR^{211′}R^{212′}R^{213′}, unsubstituted C(₁₋₆)alkyl-N⁺R^{211′}R^{212′}R^{213′}, substituted C(₁₋₆)alkyl-OR^{214′}, unsubstituted C(₁₋₆)alkyl-OR^{214′}

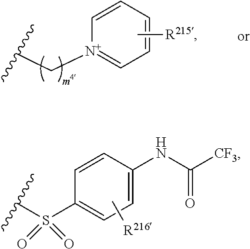

wherein m^{4′} is 1, 2, 3, 4 or 5, R^{209′}, R^{210′}, R^{214′}, R^{215′} and R^{216′} are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and R^{209′} and R^{210′}, may alternately be and independently as a pair be a 3-7 membered substituted heterocarbocyclic ring or a 3-7 membered unsubstituted heterocarbocyclic ring, and R^{211′}, R^{212′} and R^{213′} are each independently unsubstituted C(₁₋₁₁)alkyl, or substituted C(₁₋₁₁)alkyl; and R^{102′} and R^{103′} are each independently substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, or unsubstituted C(₂₋₁₁)heteroaralkyl; and R^{106′} is substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₂₋₁₁)alkyl, unsubstituted C(₆₋₁₁)aryl, unsubstituted C(₁₋₁₁)heteroaryl, unsubstituted C(₇₋₁₁)aralkyl, or unsubstituted C(₂₋₁₁)heteroaralkyl; and (b) when n is at least 2 or n²+n³ is at least 2, then a first Q¹, Q², Q⁴, Q⁵, Q⁶, Q⁷ or Q⁸ is independently selected from the group consisting of —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, and —NHC(O)R^{106′}, wherein each of R^{26′}, R^{27′}, R^{28′}, R^{100′}, R^{101′}, R^{102′}, R^{103′}, and R^{106′} is as defined above; and the remaining Q¹, Q², Q⁴, Q⁵, Q⁶, Q⁷ or Q⁸ are each independently selected from the group consisting of halogen, —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R¹⁰², —O—(C₁₋₆)alkyl-OS(O)₂R^{103′}, NO₂, NR^{104′}R^{105′}, —NHC(O)R^{106′} substituted C(₁₋₆)alkyl, substituted C(₁₋₆)heteroalkyl, unsubstituted C(₁₋₆)alkyl, and unsubstituted C(₁₋₆)heteroalkyl; wherein each R^{26′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; each of R¹⁴ and R^{105′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; or R^{104′} and R^{105′} may alternately as a pair be a 3-7 membered substituted heterocarbocyclic ring or a 3-7 membered unsubstituted heterocarbocyclic ring; each R^{106′} is substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, or unsubstituted C(₂₋₁₁)heteroaralkyl; and each of R^{27′}, R^{28′}, R^{100′}, R^{101′}, R^{102′}, and R^{103′} is as defined above; (iii) provided that when G³ is N, CH, or CG⁹ where G⁹ is C(O)OR⁹ and R⁹ is unsubstituted C(₄₋₆) alkyl, G⁴ is other than

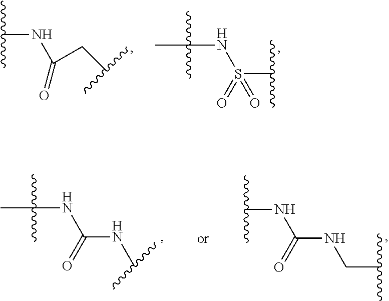

### and G⁵ is

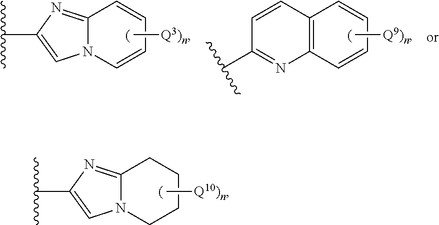

then at least one of G⁶, G⁷, and G⁸ is not H; n is at least 1; and each of Q³, Q⁹ or Q¹⁰ is independently selected from the group consisting of halogen, —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, —NHC(O)R^{106′}, substituted C(₁₋₆)alkyl, substituted C(₁₋₆)heteroalkyl, unsubstituted C(₂₋₆)alkyl, and unsubstituted C(₁₋₆)heteroalkyl; (iv) provided that when G³ is N or CH, and G⁵ is,

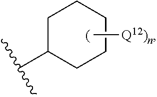

then at least one of G⁶, G⁷, and G⁸ is not H; n is at least 1; and each Q¹² is independently selected from the group consisting of halogen, —OR²⁶, —O—(C₁₋₆)alkyl-NR²⁷R²⁸, —O—(C₁₋₆)alkyl-C(O)OR^{100′}, —O—(C₁₋₆)alkyl-C(O)NHR^{106′}, —O—(C₁₋₆)alkyl-OC(O)R^{102′}, —O—(C₁₋₆)alkyl-OS(O)₂R^{103′} NO₂, —NHC(O)R^{106′}, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; (v) provided that when G³ is N or CH, and G⁴ is

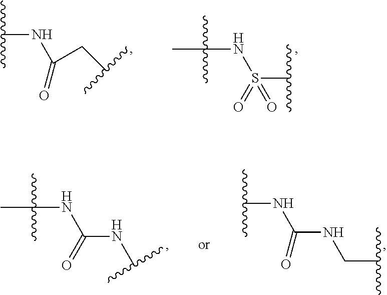 [SMILES: *CC(=O)N* ; *NS(*)(=O)=O ; *NC(=O)N* ; *CNC(=O)N*]

is: (a)

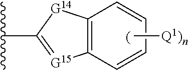

where G¹⁴ is CH₂ and G¹⁵ is N, or G¹⁴ is NH and G¹⁵ is CH, or G¹⁴ is S and G¹⁵ is CH; (b)

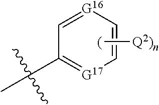

where G¹⁶ is N and G¹⁷ is N; or (c)

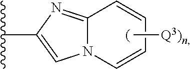

then at least one of G⁶, G⁷, and G⁸ is not H, and n is at least 1; (vi) provided that when G³ is N or CH, and G⁴ is

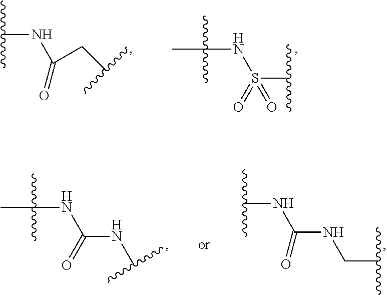 [SMILES: *CC(=O)N* ; *NS(*)(=O)=O ; *NC(=O)N* ; *CNC(=O)N*]

### and G⁵ is: (a)

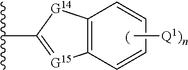

where G¹⁴ is NH and G¹⁵ is N; (b)

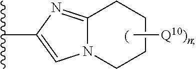

(c)

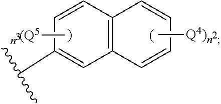

or (d)

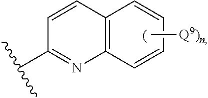

then at least one of G⁶, G⁷ and G⁸ is not H, and each of G⁶ and G⁷ is independently H, halogen, CF₃, NO₂, substituted (C₁₋₁₁)alkyl, unsubstituted (C₃₋₁₁)alkyl, substituted (C₁₋₁₁)alkoxyl, unsubstituted (C₁₋₁₁) alkoxyl, substituted (C₆₋₁₁)aryloxy, unsubstituted (C₆₋₁₁)aryloxy, C(O)OR⁵⁰, or

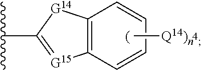

n is at least 1 or n²+n³ is at least 1; and each of Q¹, Q⁴, Q⁵, Q⁹, Q¹⁰ and Q¹² is independently selected from the group consisting of halogen, —OR²⁶, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, —NHC(O)R^{106′}, substituted C₍₁₋₆₎alkyl, and unsubstituted C₍₂₋₆₎alkyl; R^{106′} is substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₂₋₁₁)alkyl, unsubstituted C(₆₋₁₁)aryl, unsubstituted C(₁₋₁₁)heteroaryl, unsubstituted C(₇₋₁₁)aralkyl, or unsubstituted C(₂₋₁₁)heteroaralkyl; and each of R^{26′}, R^{27′}, R^{28′}, R^{100′}, R^{101′}, R^{102′}, and R^{103′} is as defined above; (vii) provided that when G³ is N or CH, and G⁴ is

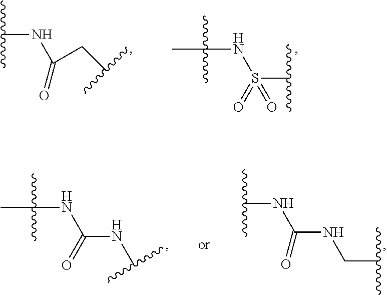 [SMILES: *CC(=O)N* ; *NS(*)(=O)=O ; *NC(=O)N* ; *CNC(=O)N*]

### and G⁵ is: (a)

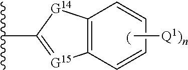

where G¹⁴ is S and G¹⁵ is N; (b)

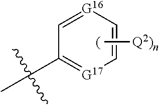

where G¹⁶ is CH and G¹⁷ is N, or G¹⁶ is N and G¹⁷ is CH, or G¹⁶ is CH and G¹⁷ is CH; (c)

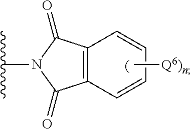

or (d) a 5-membered heteroaryl optionally substituted with (Q⁸)ₙ and containing 1 or 2 heteroatoms each heteroatom independently selected from N, O and S, then at least one of G⁶, G⁷ and G⁸ is not H, and each of G⁶ and G⁷ is independently H, halogen, CF₃, NO₂, substituted (C₁₋₁₁)alkyl, unsubstituted (C₃₋₁₁)alkyl, substituted (C₁₋₁₁)alkoxyl, unsubstituted (C₁₋₁₁) alkoxyl, substituted (C₆₋₁₁)aryloxy, unsubstituted (C₆₋₁₁)aryloxy, C(O)OR⁵⁰, or

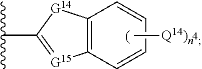

and n is at least 1; and (a) when n is 1, then each of Q¹, Q², Q⁶, or Q⁸ is independently selected from the group consisting of —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′} and —NHC(O)R^{106′}, wherein each of R²⁶, R²⁷, R^{28′}, R^{100′}, R^{101′}, R^{102′}, R^{103′} and R^{106′} is as defined above; and (b) when n is at least 2, then a first Q¹, Q², Q⁶, or Q⁸ is independently selected from the group consisting of —OR^{26′}, —O—(C₁₋₆)alkyl-NR^{27′}R²⁸, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, and —NHC(O)R¹⁰⁶, wherein each of R^{26′}, R^{27′}, R^{28′}, R^{100′}, R^{101′}, R^{102′} R^{103′}, and R^{106′} is as defined above; and the remaining Q¹, Q², Q⁶, or Q⁸ are each independently selected from the group consisting of halogen, —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, NR^{104′}R^{105′}, —NHC(O)R^{106′}, substituted C₍₁₋₆₎alkyl, substituted C₍₁₋₆₎heteroalkyl, unsubstituted C₍₁₋₆₎alkyl, and unsubstituted C₍₁₋₆₎heteroalkyl; wherein each R^{26′}, R^{27′}, R^{28′}, R^{100′}, R^{101′} R^{102′}, R^{103′}, R^{104′}, R^{105′}, and R^{106′} is as defined above; and (viii) provided that when G³ is CG⁹ and G⁹ is: (a) substituted (C₁₋₆) alkyl-NH₂; (b) unsubstituted (C₁₋₆) alkyl-NH₂; (c) substituted (C₁₋₆) alkyl-NR⁶⁴R⁶⁵ or unsubstituted (C₁₋₆) alkyl-NR⁶⁴R⁶⁵ where R⁶⁴ and R⁶⁵ as a pair are a 3-7 membered substituted heterocarbocyclic ring or a 3-7 membered unsubstituted heterocarbocyclic ring; (d) substituted (C₆₋₁₁) aryl; (e) substituted (C₁₋₁₁) heteroaryl or unsubstituted (C₁₋₁₁) heteroaryl; (f) substituted (C₆₋₁₁) arylcarbonyl or unsubstituted (C₆₋₁₁) arylcarbonyl; (g) substituted (C₁₋₁₁) heteroarylcarbonyl or unsubstituted (C₁₋₁₁) heteroarylcarbonyl; (h) —CO-substituted-carbocycle or —CO-unsubstituted-carbocycle; (i) —CO-substituted-heterocarbocycle or —CO-unsubstituted-heterocarbocycle; (j) —C(O)NR⁷R⁸ where each of R⁷ and R⁸ is CH³; (k) —C(O)NR⁷R⁸ where R⁷ is H and R⁸ is unsubstituted C₆ aryl or unsubstituted C₄ cycloalkyl; (l) —C(O)C(O)NR¹³R¹⁴ where each of R¹³ and R¹⁴ is CH₃; (m) —C(O)C(O)NR¹³R¹⁴ where each of R¹³ and R¹⁴ is

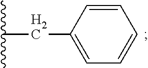 [SMILES: *Cc1ccccc1]

(n) —NR¹⁵R¹⁶ where only one of R¹⁵ and R¹⁶ is unsubstituted C₆ aryl; or (o) —NR¹⁵R¹⁶ where R¹⁵ and R¹⁶ as a pair are a 3-7 membered unsubstituted heterocarbocyclic ring, then at least one of G⁶, G⁷ and G⁸ is not H.

Illustrative embodiments of the present invention provide a method of treating a subject known to have or suspected of having a bacterial infection, the method comprising administering to the subject an effective amount of a compound selected from the group consisting of the compounds in Table 2 below, or a salt thereof, wherein the compound, or salt thereof, has anti-bacterial activity.

Illustrative embodiments of the present invention provide a method of reducing the prefalence of bacteria on a surface, the method comprising introducing a compound described herein to the surface.

Illustrative embodiments of the present invention provide use of a compound described herein for treatment of a bacterial infection.

Illustrative embodiments of the present invention provide use of a compound described herein for preparation of a medicament for treatment of a bacterial infection.

Other aspects and features of the present invention will become apparent to those ordinarily skilled in the art upon review of the following description of specific embodiments of the invention in conjunction with the accompanying figures.

### DETAILED DESCRIPTION

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁₋₁₀ or 1- to 10-membered means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups which are limited to hydrocarbon groups are termed “homoalkyl”.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

As used herein, the term “heteroatom” is meant to include oxygen (O), nitrogen (N), and sulfur (S).

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group or at the position at which the alkyl group is attached to the remainder of the molecule. Examples include, but are not limited to, —CH₂—CH₂—O—CH₃, —CH₂—C(═O)—CH₃, —CH₂—CH₂—CH₂—C(═O)—O—C(CH₃)—CH₃, —CH₂—CH₂—CH₂—C(═O)—N—CH(CH₃), —CH₂—CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, Si(CH₃)₃, —CH₂—CH═N—OCH₃, and —CH═CH—N(CH₃)—CH₃. Up to two heteroatoms may be consecutive, such as, for example, —CH₂—NH—OCH₃ and CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, unless otherwise clear from context, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Thus, a cycloalkyl or heterocycloalkyl include saturated and unsaturated ring linkages. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, and 2-piperazinyl.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁₋₄)alkyl” is meant to include, but not be limited to, trifluoromethyl, 2,2,2-trifluoroethyl, 4-chlorobutyl, 3-bromopropyl, and the like.

The term “carbocycle”, “carbocyclic” or “carbocyclic ring” by itself or in combination with another term, means, unless otherwise stated, a cyclic hydrocarbon radical, which may be fully saturated, mono- or polyunsaturated. The number of atoms in a ring of the “carbocycle”, “carbocyclic” or “carbocyclic ring” are typically defined by the number of members in the ring. For example, “C₃-7” or “3- to 7-membered” means there are 3-7 atoms in the encircling arrangement. The term “carbocycle”, “carbocyclic” or “carbocyclic ring” includes aryl moieties.

The term “heterocarbocycle”, “heterocarbocyclic” or “heterocarbocyclic ring” by itself or in combination with another term, means, unless otherwise stated, a cyclic hydrocarbon radical containing at least one heteroatom selected from the group consisting of O, N, and S. The number of atoms in a ring of the “heterocarbocycle”, “heterocarbocyclic” or “heterocarbocyclic ring” are typically defined by the number of members in the ring. For example, “C₃₋₇” or “3- to 7-membered” means there are 3-7 atoms in the encircling arrangement. The term “heterocarbocycle”, “heterocarbocyclic” or “heterocarbocyclic ring” includes heteroaryl moieties.

As used herein the term “aryl” means any moiety which has at least a portion of the moiety that conforms to Hückel's rule. This includes moieties that are hydrocarbons and moieties that include heteroatoms. For clarity, an aryl moiety as a whole does not need to conform to Hückel's rule as long as some portion of the aryl moiety, when considered in the absence of the remainder of the moiety, does conform to Hückel's rule. Non-limiting, illustrative examples of aryl moieties include phenyl, benzyl, indanyl, 1-methoxyphenyl, 2-methoxyphenyl and 1-fluorophenyl. When the terminology “C_{x-y}” is used with respect to aryl groups, the ‘C’ relates to the total number of carbon atoms in the aryl moiety and does not include the heteroatoms in the moiety. For example, 1-fluorophenyl may be described as a C₆ aryl group and 2-methoxylnaphthyl may be described as a C₁₀ aryl group.

The term “ring” as used herein means a substituted or unsubstituted cycloalkyl, substituted or unsubstituted heterocycloalkyl, substituted or unsubstituted aryl, or substituted or unsubstituted heteroaryl. A ring includes fused ring moities. The number of atoms in a ring are typically defined by the number of members in the ring. For example, a “5- to 7-membered ring” means there are 5-7 atoms in the encircling arrangement. The ring optionally includes a heteroatom. Thus, the term “5- to 7-membered ring” includes, for example pyridinyl, piperidinyl and thiazolyl rings.

As used herein, the term “substituted” refers to the replacement of a hydrogen atom on a compound with a substituent group. A substituent may be a non-hydrogen atom or multiple atoms of which at least one is a non-hydrogen atom and one or more may or may not be hydrogen atoms. For example, without limitation, substituted compounds may comprise one or more substituents selected from the group consisting of: R″, OR″, NR″R″, SR″, halogen, OC(O)R″, C(O)R″, CO₂R″, CONR″R″′, NR″′C(O)₂R″, S(O)R″, S(O)₂R″, CN and NO₂.

As used herein, each R″, R″′, and R″″ may be selected, independently, from the group consisting of: hydrogen, halogen, oxygen, substituted or unsubstituted heteroalkyl, substituted or unsubstituted aryl, substituted or unsubstituted alkyl, alkoxy or thioalkoxy groups, and arylalkyl groups with the proviso that R″, R″′, and R″″ within a substituent are not oxygen or halogen radicals bound directly to oxygen, sulfur or halogen radicals of the substituent.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″″, —N R—C(NR′R″)═NR″′, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R″′ and R″″ each preferably independently refer to hydrogen, substituted or unsubstituted heteroalkyl, substituted or unsubstituted aryl, e.g., aryl substituted with 1 to 3 halogens, substituted or unsubstituted alkyl, alkoxy or thioalkoxy groups, or arylalkyl groups. When a modulator of the invention includes more than one R group, for example, each of the R groups is independently selected as are each R′, R″, R″′ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include, unless otherwise clear from context, groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —OC(O)R′, —C(O)R′, —CO₂R′, —CONR′R″, —OC(O)NR′R″, —NR″C(O)R′, —NR′—C(O)N R″R″′, —NR″C(O)₂R′, —NR—C(NR′R″R″′)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁₋₄)alkoxy, and fluoro(C₁₋₄)alkyl, in a number ranging from zero to the total number of open valences on the aromatic ring system; and where R′, R″, R″′ and R″″ are preferably independently selected from hydrogen, alkyl, heteroalkyl, aryl and heteroaryl. When a modulator of the invention includes more than one R group, for example, each of the R groups is independently selected as are each R′, R″, R″′ and R″″ groups when more than one of these groups is present.

In one embodiment, substituents for the aryl and heteroaryl groups are varied and are selected from: halogen, —OR′, —NR′R″, —SR′, -halogen, —OC(O)R′, —C(O)R′, —CO₂R′, —CONR′R″, —OC(O)NR′R″, —NR″C(O)R′, —NR′—C(O)N R″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, —CN and —NO₂, —R′, —N₃, —CH(Ph)₂, fluoro(C₁₋₄)alkoxy, and fluoro(C₁₋₄)alkyl, in a number ranging from zero to the total number of open valences on the aromatic ring system; and where R′, R″, R″′ and R″″ are preferably independently selected from hydrogen, alkyl, heteroalkyl, aryl and heteroaryl. When a modulator of the invention includes more than one R group, for example, each of the R groups is independently selected as are each R′, R″, R″′ and R″″ groups when more than one of these groups is present

In some embodiments of the present invention, substituted alkyl by itself, or in combination with another term, may be substituted with at least one substituent independently selected from the group consisting of -Me, —OH, —NH₂, —NHMe, —NMe₂, —CO₂H, —CONH₂, ═O, —OMe, —OEt, -Ph, -pyridyl,

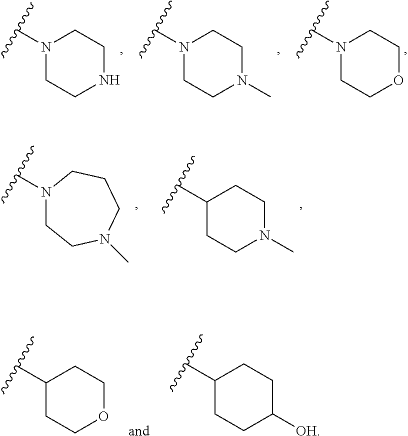

In some embodiments of the present invention, substituted heteroalkyl by itself, or in combination with another term, may be substituted with at least one substituent independently selected from the group consisting of -Me, —OH, —NH₂, —NHMe, —NMe₂, —CO₂H, —CONH₂, ═O, —OMe, —OEt, -Ph, -pyridyl,

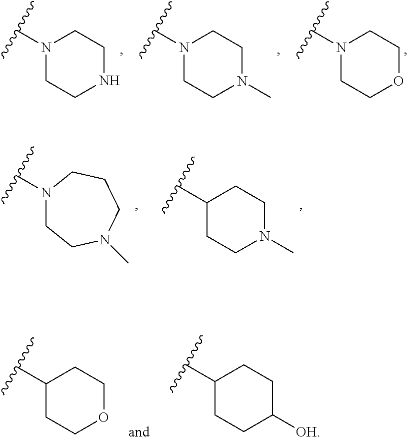

In some embodiments of the present invention, substituted aryl by itself, or in combination with another term, may be substituted with at least one substituent independently selected from the group consisting of F, Cl, Br, OMe and OH.

“Moiety” refers to the radical of a molecule that is attached to another moiety.

As used herein, the symbol

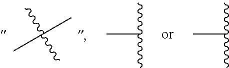

indicates the point at which the displayed moiety is attached to the remainder of the molecule. For example, CH₃-(moiety), wherein moiety is

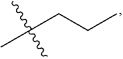 [SMILES: *CCC]

would mean CH₃—CH₂—CH₂—CH₃.

In some embodiments of the invention, there is provided a compound of formula (1), use of a compound of formula (1), or a method of treating a subject known to have or suspected of having a bacterial infection, the method comprising administering to the subject an effective amount of a compound having a structure of formula (1):

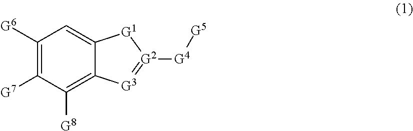

or a salt thereof.

In some embodiments of formula (1), G¹ is NH, O, or S. In some embodiments, G¹ is NH or S. In some embodiments, G¹ is S. In some embodiments G¹ is NH.

In some embodiments of formula (1), G², G³ and G⁴ may either: i) together form a ring moiety selected from the group consisting of:

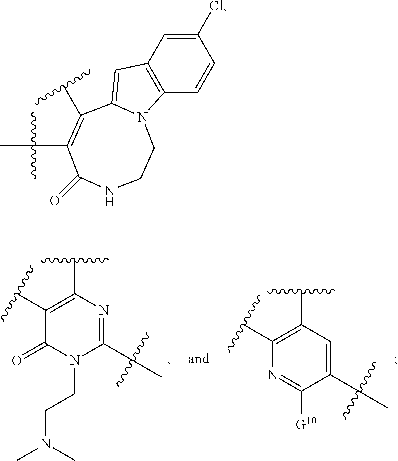

or
ii) together do not form a ring moiety wherein G² is C; G³ is N, CH or CG⁹; and

G is selected from the group consisting of: a bond,

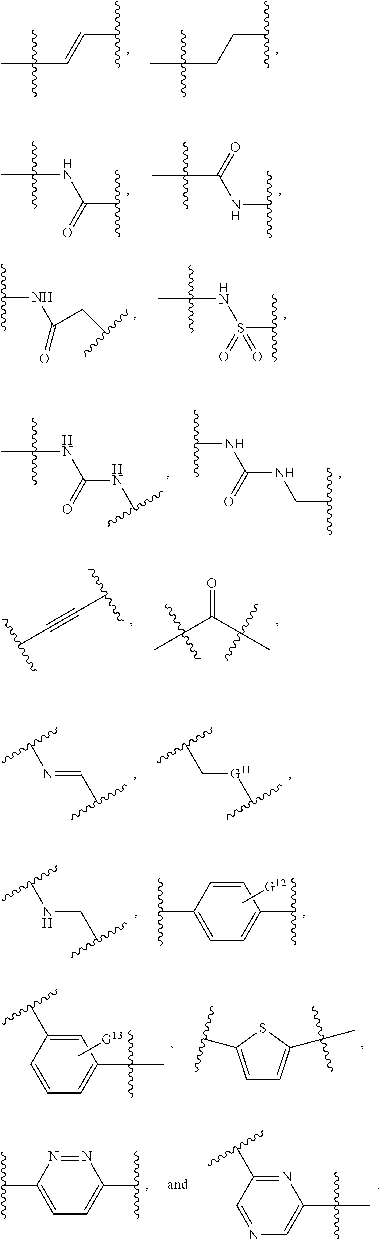

In embodiments of formula (1) in which G², G³ and G⁴ together form the ring moiety

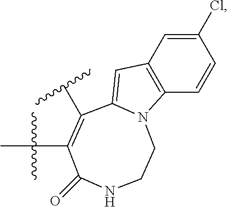 [SMILES: */C1=C(\*)c2cc3cc(Cl)ccc3n2CCNC1=O]

G⁵ is absent. Further, G⁵ is only absent from compounds of formula (1) when G², G³ and G⁴ together form this ring moiety.

In some embodiments of formula (1), G³ is CG⁹ or CH. In some embodiments, G³ is CG⁹. In some embodiments, G³ is CH.

In some embodiments of formula (1), G⁴ is selected from the group consisting of: a bond,

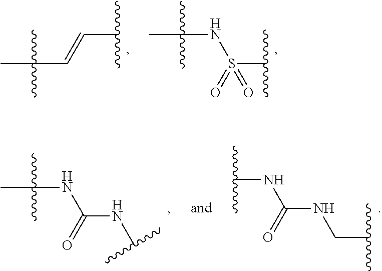

In some embodiments, G⁴ is selected from the group consisting of

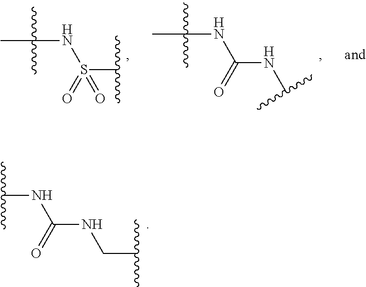 [SMILES: *NS(*)(=O)=O ; *NC(=O)N* ; *CNC(=O)N*]

In some embodiments, G⁴ is selected from the group consisting of: a bond, and

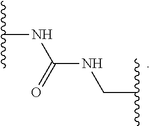 [SMILES: *CNC(=O)N*]

In some embodiments, G⁴ is a bond. In some embodiments, G⁴ is

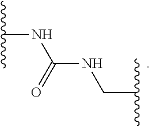 [SMILES: *CNC(=O)N*]

In some embodiments of formula (1), G⁵ is absent,

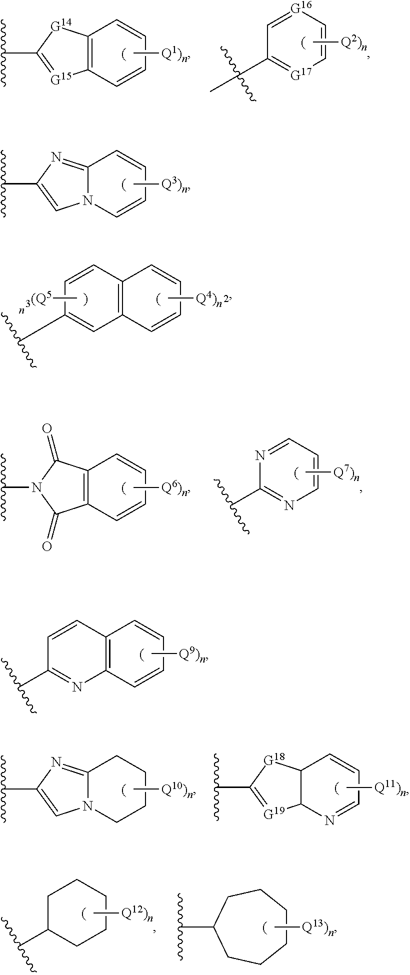

a 5-membered heteroaryl optionally substituted with (Q⁸)ₙ and containing 1 or 2 heteroatoms each heteroatom independently selected from N, O and S, substituted (C₁₋₁₁)alkyl, unsubstituted (C₁₋₁₁)alkyl, substituted (C₁₋₁₁)heteroalkyl, unsubstituted (C₁₋₁₁)heteroalkyl, substituted (C₃₋₁₁)heterocycloalkyl, unsubstituted (C₃₋₁₁)heterocycloalkyl, substituted (C₈₋₉)cycloalkyl, or unsubstituted (C₈₋₉)cycloalky. In some embodiments of formula (1), G⁵ is absent,

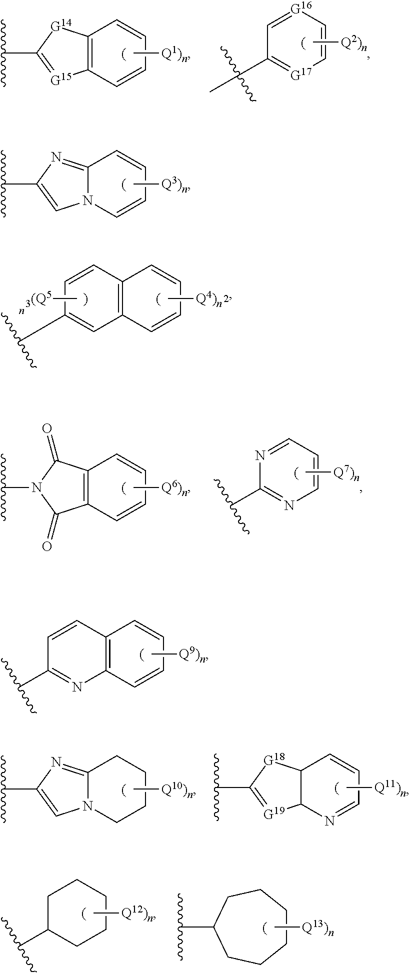

or a 5-membered heteroaryl optionally substituted with (Q⁸)ₙ and containing 1 or 2 heteroatoms each heteroatom independently selected from N, O and S. In some embodiments, G⁵ is

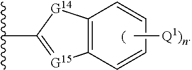

In some embodiments, G⁵ is selected from the group consisting of:

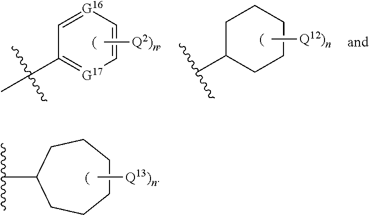

In some embodiments, G⁵ is

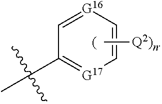

In some embodiments of formula (1), G⁶ is H, halogen, CF₃, NO₂, substituted (C₁₋₁₁)alkyl, unsubstituted (C₁₋₁₁)alkyl, substituted (C₁₋₁₁)alkoxyl, unsubstituted (C₁₋₁₁) alkoxyl, substituted (C₆₋₁₁)aryloxy, unsubstituted (C₆₋₁₁)aryloxy, C(O)OR⁵⁰, substituted (C₁₋₁₁)heteroalkyl, unsubstituted (C₁₋₁₁) heteroalkyl or

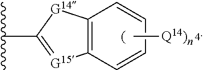

In some embodiments of formula (1), G⁶ is H, halogen, CF₃, NO₂, substituted (C₁₋₁₁)alkyl, unsubstituted (C₁₋₁₁)alkyl, substituted (C₁₋₁₁)alkoxyl, unsubstituted (C₁₋₁₁) alkoxyl, substituted (C₆₋₁₁)aryloxy, unsubstituted (C₆₋₁₁)aryloxy, C(O)OR⁵⁰, or

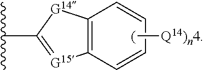

In some embodiments of formula (1), G⁷ is H, halogen, CF₃, NO₂, substituted (C₁₋₁₁)alkyl, unsubstituted (C₁₋₁₁)alkyl, substituted (C₁₋₁₁) alkoxyl, unsubstituted (C₁₋₁₁) alkoxy, substituted (C₆₋₁₁)aryloxy, unsubstituted (C₆₋₁₁)aryloxy, C(O)OR⁵¹, substituted (C₁₋₁₁)heteroalkyl, unsubstituted (C₁₋₁₁) heteroalkyl, or

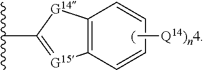

In some embodiments, G⁷ is H, halogen, CF₃, NO₂, substituted (C₁₋₁₁)alkyl, unsubstituted (C₁₋₁₁)alkyl, substituted (C₁₋₁₁) alkoxyl, unsubstituted (C₁₋₁₁) alkoxy, substituted (C₆₋₁₁)aryloxy, unsubstituted (C₆₋₁₁)aryloxy, C(O)OR⁵¹, or

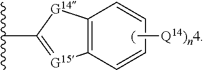

In some embodiments of formula (1), R⁵⁰ and R⁵¹ are each independently substituted (C₁₋₆)alkyl, unsubstituted (C₁₋₆)alkyl, substituted (C₁₋₆)heteroalkyl or unsubstituted (C₁₋₆) heteroalkyl.

In some embodiments of formula (1), G⁸ is H, C(═O)N(CH₃)₂, or C(═O)N(H)C(H₂)C₆H₅.

In some embodiments of formula (1), G⁹ is —CN, CF₃, —SO₂NH₂, —NH₂, —C(CF₃)₂OH, —C(CF₃)(H)OH, —C(CF₃)(CH₃)OH, —C(NOH)C(R²¹)(R²²)(R²³), C(NOH)N(R²⁴)(R²⁵), C(NOR⁶⁰)C(R⁶¹)(R⁶²)(R⁶³), substituted (C₁₋₆) alkyl-NR⁶⁴R⁶⁵, unsubstituted (C₁₋₆) alkyl-NR⁶⁴R⁶⁵, substituted (C₆₋₁₁) aryl, unsubstituted (C₆₋₁₁)aryl, substituted (C₁₋₁₁) heteroaryl, unsubstituted (C₁₋₁₁) heteroaryl, substituted (C₆₋₁₁) arylcarbonyl, unsubstituted (C₆₋₁₁) arylcarbonyl, substituted (C₁₋₁₁) heteroarylcarbonyl, unsubstituted (C₁₋₁₁) heteroarylcarbonyl, —CO-substituted-carbocycle, —CO-unsubstituted-carbocycle, —CO-substituted-heterocarbocycle, —CO-unsubstituted-heterocarbocycle, —CO-substituted-C(₁₋₆)alkyl-OR¹, —CO-unsubstituted-C(₁₋₆)alkyl-OR¹, —CO-substituted-C(₁₋₆)alkyl-NR²R³, —CO-unsubstituted-C(₁₋₆)alkyl-NR²R³, —CO-substituted-C(₁₋₆)alkyl-C(O)OR⁴, —CO-unsubstituted-C(₁₋₆)alkyl-C(O)OR⁴, —CO-substituted-C(₁₋₆)alkyl-C(O)NR⁵R⁶, —CO-unsubstituted-C(₁₋₆)alkyl-C(O)NR⁵R⁶, —C(O)NR⁷R⁸, —C(O)OR⁹, —C(O)C(O)OR¹², —C(O)C(O)NR¹³R¹⁴, —NR¹⁵R¹⁶, —N(H)C(O)substituted-C(₁₋₆)alkyl, —N(H)C(O)unsubstituted-C(₁₋₆)alkyl, —N(H)C(O)substituted-C(₁₋₆)haloalkyl, —N(H)C(O)unsubstituted-C(₁₋₆)haloalkyl, —N(H)C(O)substituted-C(₆₋₁₁)aryl, —N(H)C(O)unsubstituted-C(₆₋₁₁)aryl, —N(H)C(O)substituted-C(₁₋₁)heteroaryl, —N(H)C(O)unsubstituted-C(₁₋₁₁)heteroaryl, —N(H)C(O)NR¹⁷R¹⁸, —N(H)CO-substituted-C(₁₋₆)alkyl-OR¹⁹, —N(H)CO-unsubstituted-C(₁₋₆)alkyl-OR¹⁹, each of R¹, R², R³, R⁴, R⁵, R⁶, R¹², R¹³, R¹⁴, R¹⁵, R¹⁶, R¹⁷, R¹⁸, R¹⁹, R²⁴, and R²⁵ is independently selected from the group consisting of: H, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl, each of R²¹, R²², R²³, R⁶¹, R⁶² and R⁶³ is independently selected from the group consisting of: H, F, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl. Each of R⁶⁴ and R⁶⁵ is independently selected from the group consisting of: H, substituted C(₃₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl. Each pair: a) R² and R³, b) R⁵ and R⁶, c) R¹³ and R¹⁴, d) R¹⁵ and R¹⁶, e) R¹⁷ and R¹⁸, and f) R⁶⁴ and R⁶⁵ may alternately be and independently as a pair be a 3-7 membered substituted heterocarbocyclic ring or a 3-7 membered unsubstituted heterocarbocyclic ring. R⁶⁰ is unsubstituted C(₁₋₁₁)alkyl, substituted C(₁₋₁₁)alkyl, unsubstituted C(₁₋₁₁)alkyl-NR⁶⁶R⁶⁷, substituted C(₁₋₁₁)alkyl-NR⁶⁶R⁶⁷, unsubstituted C(₁₋₁₁)alkyl-N⁺R⁶⁸R⁶⁹R⁷⁰, or substituted C(₁₋₁₁)alkyl-N⁺R⁶⁸R⁶⁹R⁷⁰, wherein R⁶⁶ and R are each independently H, unsubstituted C(₁₋₁₁)alkyl or substituted C(₁₋₁₁)alkyl, and R⁶⁸, R⁶⁹ and R⁷⁰ are each independently unsubstituted C(₁₋₁₁)alkyl, or substituted C(₁₋₁₁)alkyl, each of R⁷ and R⁸ are either I) independently selected from the group consisting of: H, substituted C(₁₋₆)alkyl, substituted C(₁₋₆)alkyl-NR⁵²R⁵³ unsubstituted C(₁₋₆)alkyl-NR⁵²R⁵³, substituted C(₆)alkyl-N⁺R⁷¹R⁷²R⁷³, unsubstituted C(₁₋₆)alkyl-N⁺R⁷¹R⁷²R⁷³, substituted C(₁₋₆)alkyl-OC(O)unsubstituted C(₁₋₆)alkyl-NR⁷⁴R⁷⁵, unsubstituted C(₁₋₆)alkyl-OC(O)unsubstituted C(₁₋₆)alkyl-NR⁷⁴R⁷⁵, substituted C(₁₋₆)alkyl-C(O)NHS(O)₂R⁷⁶, unsubstituted C(₁₋₆)alkyl-C(O)NHS(O)₂R⁷⁶, substituted C(₆₋₁₁)aryl, substituted C(₃₋₁₁)carbocyclic, substituted C(₄₋₇)heterocarbocycle, substituted C(₄₋₇)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₃₋₁₁)carbocyclic, unsubstituted C(₁₋₁₁)heterocarbocycle, unsubstituted C(₁₋₁₁)heteroaryl, unsubstituted C(₇₋₁₁)aralkyl, and unsubstituted C(₂₋₁₁)heteroaralkyl wherein each of R⁵², R⁵³, R⁷⁴ and R⁷⁵ is selected from the group consisting of: H, unsubstituted C(₁₋₆)alkyl, substituted C(₃₋₇)heterocycloalkyl, unsubstituted C(₃₋₇)heterocycloalkyl, substituted C(₁₋₆)alkyl, substituted C(₃₋₇)cycloalkyl and unsubstituted C(₃₋₇)cycloalkyl, or each pair: a) R⁵² and R⁵³, or (b) R⁷⁴ and R⁷⁵, together form a 3-7 membered substituted heterocarbocyclic ring or a 3-7 membered unsubstituted heterocarbocyclic ring, and wherein each of R⁷¹, R⁷², R⁷³ and R⁷⁶ is independently unsubstituted C(₁₋₁₁)alkyl, or substituted C(₁₋₁₁)alkyl, or II) together form a 3-7 membered substituted heterocarbocyclic ring or a 3-7 membered unsubstituted heterocarbocyclic ring. R⁹ is selected from the group consisting of substituted C(₁₋₆)alkyl, substituted C(₁₋₆)alkyl-NR¹⁰R¹¹, unsubstituted C(₁₋₆)alkyl-NR¹⁰R¹¹, substituted C(₁₋₆)alkyl-OR²⁰, unsubstituted C(₁₋₆)alkyl-OR²⁰, and unsubstituted C(₁₋₆)alkyl wherein each of R¹⁰, R¹¹ and R²⁰ is independently selected from the group consisting of: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl. R¹⁰ and R¹¹ may alternately as a pair be a 3-7 membered substituted heterocarbocyclic ring or a 3-7 membered unsubstituted heterocarbocyclic ring, or G⁹ is

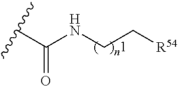

wherein n¹ is 1, 2, 3 or 4 and R⁵⁴ is

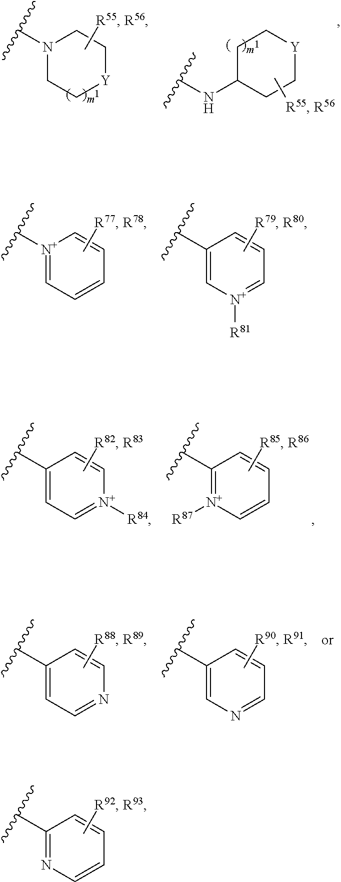

wherein m¹=0, 1 or 2, R⁵⁵ and R⁵⁶ are independently H, carbonyl (═O), Me, Ph, CO₂R⁹⁴, CO₂NH₂, C(₁₋₆)substituted alkyl or C(₁₋₆)unsubstituted alkyl, wherein R⁹⁴ is H, C(₁₋₆)unsubstituted alkyl or C(₁₋₆)substituted alkyl.

In some embodiments of formula (1), G⁹ is CF₃, —SO₂NH₂, —NH₂, —C(CF₃)₂OH, —C(CF₃)(H)OH, —C(CF₃)(CH₃)OH, —C(NOH)C(R²¹)(R²²)(R²³), C(NOH)N(R²⁴)(R²⁵), C(NOR⁶⁰)C(R⁶¹)(R⁶²)(R⁶³), substituted (C₁₋₆) alkyl-NR⁶⁴R⁶⁵, unsubstituted (C₁₋₆) alkyl-NR⁶⁴R⁶⁵, substituted (C₆₋₁₁) aryl, unsubstituted (C₁₀)aryl, substituted (C₁₋₁₁) heteroaryl, unsubstituted (C₁₋₁₁) heteroaryl, substituted (C₆₋₁₁) arylcarbonyl, unsubstituted (C₆₋₁₁) arylcarbonyl, substituted (C₁₋₁₁) heteroarylcarbonyl, unsubstituted (C₁₋₁₁) heteroarylcarbonyl, —CO-substituted-carbocycle, —CO-unsubstituted-carbocycle, —CO-substituted-heterocarbocycle, —CO-unsubstituted-heterocarbocycle, —CO-substituted-C (1-6)alkyl-OR¹, —CO-unsubstituted-C(₁₋₆)alkyl-OR¹, —CO-substituted-C(₁₋₆)alkyl-NR²R³, —CO-unsubstituted-C(₁₋₆)alkyl-NR²R³, —CO-substituted-C(₁₋₆)alkyl-C(O)OR⁴, —CO-unsubstituted-C(₁₋₆)alkyl-C(O)OR⁴, —CO-substituted-C(₁₋₆)alkyl-C(O)NR⁵R⁶, —CO-unsubstituted-C(₁₋₆)alkyl-C(O)NR⁵R⁶, —C(O)NR⁷R⁸, —C(O)OR⁹, —C(O)C(O)OR¹², —C(O)C(O)NR¹³R¹⁴, —NR¹⁵R¹⁶, —N(H)C(O)substituted-C(₁₋₆)alkyl, —N(H)C(O)unsubstituted-C(₁₋₆)alkyl, —N(H)C(O)substituted-C(₁₋₆)haloalkyl, —N(H)C(O)unsubstituted-C(₁₋₆)haloalkyl, —N(H)C(O)substituted-C(₆₋₁₁)aryl, —N(H)C(O)unsubstituted-C(₆₋₁₁)aryl, —N(H)C(O)substituted-C(₁₋₁)heteroaryl, —N(H)C(O)unsubstituted-C(₁₋₁₁₎heteroaryl, —N(H)C(O)NR¹⁷R¹⁸, —N(H)CO-substituted-C(₁₋₆)alkyl-OR¹⁹, —N(H)CO-unsubstituted-C(₁₋₆)alkyl-OR¹⁹, each of R¹, R², R³, R⁴, R⁵, R⁶, R¹², R¹³, R¹⁴, R¹⁷, R¹⁸, R¹⁹, R²⁴, and R²⁵ is independently selected from the group consisting of: H, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl, and each of R²¹, R²², R²³, R⁶¹, R⁶² and R⁶³ is independently selected from the group consisting of: H, F, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl. Each pair: a) R² and R³, b) R⁵ and R⁶, c) R¹³ and R¹⁴, and d) R¹⁷ and R¹⁸ may alternately be and independently as a pair be a 3-7 membered substituted heterocarbocyclic ring or a 3-7 membered unsubstituted heterocarbocyclic ring. R⁶⁰ is unsubstituted C(₁₋₁₁)alkyl, substituted C(₁₋₁₁)alkyl, unsubstituted C(₁₋₁₁)alkyl-NR⁶⁶R⁶⁷, substituted C(₁₋₁₁)alkyl-NR⁶⁶R⁶⁷, unsubstituted C(₁₋₁₁)alkyl-N⁺R⁶⁸R⁶⁹R⁷⁰, or substituted C(₁₋₁₁)alkyl-N⁺R⁶⁸R⁶⁹R⁷⁰, wherein R⁶⁶ and R⁶⁷ are each independently H, unsubstituted C(₁₋₁₁)alkyl or substituted C(₁₋₁₁)alkyl, and R⁶⁸, R⁶⁹ and R⁷⁰ are each independently unsubstituted C(₁₋₁₁)alkyl, or substituted C(₁₋₁₁)alkyl, each of R¹⁵ and R¹⁶ is independently selected from the group consisting of: H, substituted C(₁₋₆)alkyl, substituted C(₁₋₁₁)aryl, substituted C(₁₋₁₁)heteroaryl, substituted C(₇₋₁₁)aralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl, or R¹⁵ and R¹⁶ may alternately be a 3-7 membered unsubstituted heterocarbocyclic ring. Each of R⁶⁴ and R⁶⁵ is independently selected from the group consisting of: H, substituted C(₃₋₆)alkyl, substituted C(₁₋₁₁)aryl, substituted C(₁₋₁₁)heteroaryl, substituted C(₇₋₁₁)aralkyl, unsubstituted C(₂₋₁₁)alkyl, unsubstituted C(₁₋₁₁)aryl, unsubstituted C(₁₋₁₁)heteroaryl, and unsubstituted C(₈₋₁₁)aralky, or R⁶⁴ and R⁶⁵ may alternately be a 3-7 membered substituted heterocarbocyclic ring or a 3-7 membered unsubstituted heterocarbocyclic ring. Each of R⁷ and R⁸ are either I) independently selected from the group consisting of: H, substituted C(₁₋₆)alkyl, substituted C(₁₋₆)alkyl-NR⁵²R⁵³, unsubstituted C(₁₋₆)alkyl-NR⁵²R⁵³, substituted C(₁₋₆)alkyl-N⁺R⁷¹R⁷²R⁷³, unsubstituted C(₁₋₆)alkyl-N⁺R⁷¹R⁷²R⁷³, substituted C(₁₋₆)alkyl-OC(O)unsubstituted C(₁₋₆)alkyl-NR⁷⁴R⁷⁵, unsubstituted C(₁₋₆)alkyl-OC(O)unsubstituted C(₁₋₆)alkyl-NR⁷⁴R⁷⁵, substituted C(₁₋₆)alkyl-C(O)NHS(O)₂R⁷⁶, unsubstituted C(₁₋₆)alkyl-C(O)NHS(O)₂R⁷⁶, substituted C(₆₋₁₁)aryl, substituted C(₃₋₁₁)carbocyclic, substituted C(₄₋₇)heterocarbocycle, substituted C(₄₋₇)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₃₋₁₁)carbocyclic, unsubstituted C(₁₋₁₁)heterocarbocycle, unsubstituted C(₁₋₁₁)heteroaryl, unsubstituted C(₇₋₁₁)aralkyl, and unsubstituted C(₂₋₁₁)heteroaralkyl wherein each of R⁵², R⁵³, R⁷⁴ and R⁷⁵ is selected from the group consisting of: H, unsubstituted C(₁₋₆)alkyl, substituted C(₃₋₇)heterocycloalkyl, unsubstituted C(₃₋₇)heterocycloalkyl, substituted C(₁₋₆)alkyl, substituted C(₃₋₇)cycloalkyl and unsubstituted C(₃₋₇)cycloalkyl, or each pair: a) R⁵² and R⁵³, or (b) R⁷⁴ and R⁷⁵, together form a 3-7 membered substituted heterocarbocyclic ring or a 3-7 membered unsubstituted heterocarbocyclic ring, and wherein each of R⁷¹, R⁷², R⁷³ and R⁷⁶ is independently unsubstituted C(₁₋₁₁)alkyl, or substituted C(₁₋₁₁)alkyl, or II) together form a 3-7 membered substituted heterocarbocyclic ring or a 3-7 membered unsubstituted heterocarbocyclic ring. R⁹ is selected from the group consisting of substituted C(₁₋₆)alkyl, substituted C(₁₋₆)alkyl-NR¹⁰R¹¹, unsubstituted C(₁₋₆)alkyl-NR¹⁰R¹¹, substituted C(₁₋₆)alkyl-OR²⁰, unsubstituted C(₁₋₆)alkyl-OR²⁰, and unsubstituted C(₄₋₆)alkyl wherein each of R¹⁰, R¹¹ and R²⁰ is independently selected from the group consisting of: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl. R¹⁰ and R¹¹ may alternately as a pair be a 3-7 membered substituted heterocarbocyclic ring or a 3-7 membered unsubstituted heterocarbocyclic ring, or G⁹ is

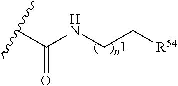

wherein n¹ is 1, 2, 3 or 4 and R⁵⁴ is

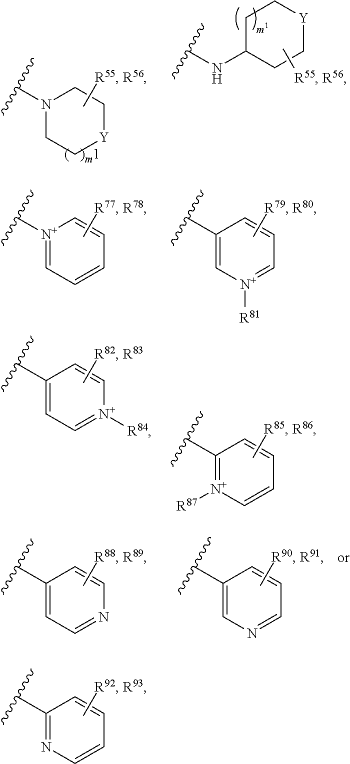

wherein m¹=0, 1 or 2, R⁵⁵ and R⁵⁶ are independently H, carbonyl (═O), Me, Ph, CO₂R⁹⁴, CO₂NH₂, C(₁₋₆)substituted alkyl or C(₁₋₆)unsubstituted alkyl, wherein R⁹⁴ is H, C(₁₋₆)unsubstituted alkyl or C(₁₋₆)substituted alkyl. R⁷⁷, R⁷⁸, R⁷⁹, R⁸⁰, R⁸², R⁸³, R⁸⁵, R⁸⁶, R⁸⁸, R⁸⁹, R⁹⁰, R⁹¹, R⁹² and R⁹³ are each independently H, C(₁₋₆)substituted alkyl, C(₁₋₆)unsubstituted alkyl, substituted C(₁₋₆)heteroalkyl, unsubstituted C₍₁₋₆₎ heteroalkyl, OR⁹⁵, C(O)R⁹⁶, or NR⁹⁷R⁹⁸, wherein R⁹⁵ is H, C(₁₋₆)substituted alkyl, or C(₁₋₆)unsubstituted alkyl, R⁹⁶ is C(₁₋₆)substituted alkyl, or C(₁₋₆)unsubstituted alkyl, and R⁹⁷ and R⁹⁸ are each independently H, C(₁₋₆)substituted alkyl, or C(₁₋₆)unsubstituted alkyl, or each pair: a) R⁷⁷ and R⁷⁸, b) R⁷⁹ and R⁸⁰, c) R⁸² and R⁸³, d) R⁸⁵ and R⁸⁶, e) R⁸⁸ and R⁸⁹, f) R⁹⁰ and R⁹¹, or g) R⁹² and R⁹³ are attached to adjacent ring-forming C atoms, and together with the ring-forming C atoms, form a substituted C₆ aryl ring or an unsubstituted C₆ aryl ring. R⁸¹, R⁸⁴ and R⁸⁷ each independently is C(₁₋₆)substituted alkyl, or C(₁₋₆)unsubstituted alkyl. Y is CH₂, CHOH, CHO—CO—C(₁₋₆)unsubstituted alkyl, CHO—CO—C(₁₋₆)substituted alkyl, NCONH₂, N—C(₁₋₆)substituted alkyl, N—C(₁₋₆)unsubstituted alkyl, NH or N—C(O)OR⁹⁹, wherein R⁹⁹ is C(₁₋₆)unsubstituted alkyl, C(₁₋₆)substituted alkyl, C(₆₋₁₁)unsubstituted aralkyl or C(₆₋₁₁)substituted aralkyl.

In some embodiments of formula (1), G⁹ is —C(NOH)C(R²¹)(R²²)(R²³) or C(NOH)N(R²⁴)(R²⁵).

In some embodiments of formula (1), R²¹, R²² and R²³ are each F.

In some embodiments of formula (1), R²⁴ and R²⁵ are H.

In some embodiments of formula (1), G¹⁰ is selected from the group consisting of: a straight C(₁₋₆)alkyl, a branched C(₃₋₆)alkyl and phenyl.

In some embodiments of formula (1), G¹¹ is NHCH₂, NH, NHCO, SCH₂, O, or S.

In some embodiments of formula (1), G¹² is H, NO₂, or OMe.

In some embodiments of formula (1), G¹³ is H, NO₂, or OMe.

In some embodiments of formula (1), G¹⁴, is NH, S, O, N—CH₃, N—CH₂—OCH₃, N—CH₂—COOH, N—CH₂—CH₂OH, N—CH₂—C(O)NH₂, CH—CH₃, N—R¹⁴, CH—R¹⁴ or substituted C(₁₋₆)alkyl-NR⁵²R⁵³, wherein R^{14′} is C₍₁₋₆₎ substituted alkyl, C₍₁₋₆₎ unsubstituted alkyl,

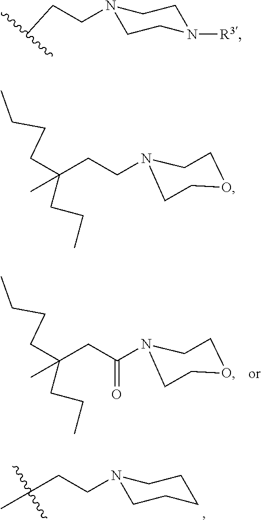

wherein R^{3′} is H, unsubstituted alkyl, or substituted alkyl, wherein the alkyl is 1-6 carbons in length, and the alkyl is optionally substituted with Br, F, Cl, I, OH, OMe, or N₃. In some embodiments of formula (1), G¹⁴ is NH.

In some embodiments of formula (1), G^{14′} is NH, S, O, N—CH₃, N—CH₂—OCH₃, N—CH₂—COOH, N—CH₂—CH₂OH, N—CH₂—C(O)NH₂, CH—CH₃, N—R¹⁴, CH—R¹⁴ or substituted C(₁₋₆)alkyl-NR⁵²R⁵³, wherein R^{14′} is C₍₁₋₆₎ substituted alkyl, C₍₁₋₆₎ unsubstituted alkyl,

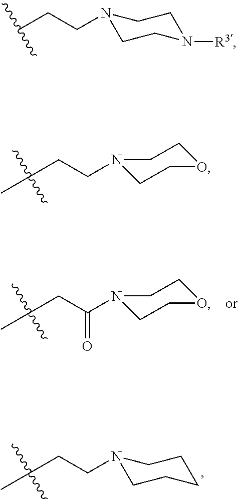

wherein R^{3′} is H, unsubstituted alkyl, or substituted alkyl, wherein the alkyl is 1-6 carbons in length, and the alkyl is optionally substituted with Br, F, Cl, I, OH, OMe, or N₃.

In some embodiments of formula (1), G¹⁵ is N, CH or CG⁹. In some embodiments, G¹⁵ is CH.

In some embodiments of formula (1), G¹⁵ is N, CH or CG⁹.

In some embodiments of formula (1), G¹⁶ is N or CH. In some embodiments G¹⁶ is CH.

In some embodiments of formula (1), G¹⁷ is N or CH. In some embodiments C¹⁷ is CH.

In some embodiments of formula (1), G¹⁸ is NH, S, O, N—CH₃, N—CH₂—OCH₃, N—CH₂—COOH, N—CH₂—CH₂OH, N—CH₂—C(O)NH₂, CH—CH₃, N—R^{14′}, CH—R^{14′} or substituted C(₁₋₆)alkyl-NR⁵²R⁵³, wherein R^{14′} is C₍₁₋₆₎ substituted alkyl, C₍₁₋₆₎ unsubstituted alkyl,

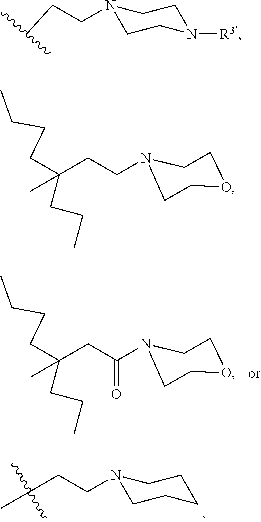

wherein R^{3′} is H, unsubstituted alkyl, or substituted alkyl, wherein the alkyl is 1-6 carbons in length, and the alkyl is optionally substituted with Br, F, Cl, I, OH, OMe, or N₃.

In some embodiments of formula (1), G¹⁹ is N, CH or CG⁹.

In some embodiments of formula (1), each of n, n², n³ and n⁴ is independently 0, 1, 2, 3, or 4. In some embodiment of formula (1), n is 0. In some embodiments of formula (1), n is 1. In some embodiments of formula (1), n is 2. In some embodiments of formula (1), n is 3. In some embodiments of formula (1), n is 4. In some embodiments of formula (1), n is at least 1. In some embodiments of formula (1), n is at least 2.

In some embodiments of formula (1), each Q¹ and Q¹⁴ is independently selected from the group consisting of: halogen, —OR²⁶, —O—(C₁₋₆)alkyl-NR²⁷R²⁸, —O—(C₁₋₆)alkyl-C(O)OR¹⁰⁰, —O—(C₁₋₆)alkyl-C(O)NHR¹⁰¹, —O—(C₁₋₆)alkyl-OC(O)R¹⁰², —O—(C₁₋₆)alkyl-OS(O)₂R¹⁰³, NO₂, NR¹⁰⁴R¹⁰⁵, —NHC(O)R¹⁰⁶, substituted C(₁₋₆)alkyl, substituted C(₁₋₆)heteroalkyl, unsubstituted C(₁₋₆)alkyl, and unsubstituted C(₁₋₆)heteroalkyl. In some embodiments, at least one Q¹ is selected from the group consisting of: —OR²⁶, —O—(C₁₋₆)alkyl-NR²⁷R²⁸, —O—(C₁₋₆)alkyl-C(O)OR¹⁰⁰, —O—(C₁₋₆)alkyl-C(O)NHR¹⁰¹, —O—(C₁₋₆)alkyl-OC(O)R¹⁰², and —O—(C₁₋₆)alkyl-OS(O)₂R¹⁰³. In some embodiments, at least one Q¹ is halogen. In some embodiments, at least one Q¹ is —O—(C₁₋₆)alkyl-C(O)NHR¹⁰¹. In some embodiments, at least one Q¹ is Cl.

In some embodiments of formula (1), each Q² is independently selected from the group consisting of: halogen, —OR²⁹, —O—(C₁₋₆)alkyl-NR³⁰R³¹, —O—(C₁₋₆)alkyl-C(O)OR¹⁰⁷, —O—(C₁₋₆)alkyl-C(O)NHR¹⁰⁸, —O—(C₁₋₆)alkyl-OC(O)R¹⁰⁹, —O—(C₁₋₆)alkyl-OS(O)₂R¹¹⁰, NO₂, NR¹¹¹R¹¹², —NHC(O)R¹¹³, substituted C₍₁₋₆₎alkyl, substituted C₍₁₋₆₎heteroalkyl, unsubstituted C₍₁₋₆₎alkyl, and unsubstituted C₍₁₋₆₎heteroalkyl. In some embodiments, Q² is selected from the group consisting of: halogen, NR¹¹¹R¹¹², NHC(O)R¹¹³, and substituted C₍₁₋₆₎ alkyl. In some embodiments, at least one Q² is halogen.

In some embodiments of formula (1), each Q³ is independently selected from the group consisting of: halogen, —OR¹¹⁴, —O—(C₁₋₆)alkyl-NR¹⁵R¹¹⁶, —O—(C₁₋₆)alkyl-C(O)OR¹¹⁷, —O—(C₁₋₆)alkyl-C(O)NHR⁸, —O—(C₁₋₆)alkyl-OC(O)R¹¹⁹, —O—(C₁₋₆)alkyl-OS(O)₂R¹²⁰, NO₂, NR¹²¹R¹²², —NHC(O)R¹²³, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;

In some embodiments of formula (1), each Q⁴ is independently selected from the group consisting of: halogen, —OR³⁵, —O—(C₁₋₆)alkyl-NR³⁶R³⁷, —O—(C₁₋₆)alkyl-C(O)OR¹²⁴, —O—(C₁₋₆)alkyl-C(O)NHR¹²⁵, —O—(C₁₋₆)alkyl-OC(O)R¹²⁶, —O—(C₁₋₆)alkyl-OS(O)₂R¹²⁷, NO₂, NR¹²⁸R¹²⁹, —NHC(O)R¹³⁰, substituted C₍₁₋₆₎alkyl, substituted C₍₁₋₆₎heteroalkyl, unsubstituted C₍₁₋₆₎alkyl, and unsubstituted C₍₁₋₆₎heteroalkyl.

In some embodiments of formula (1), each Q⁵ is independently selected from the group consisting of: halogen, —OR³⁸, —O—(C₁₋₆)alkyl-NR³⁹R⁴⁰, —O—(C₁₋₆)alkyl-C(O)OR¹³¹, —O—(C₁₋₆)alkyl-C(O)NHR³², —O—(C₁₋₆)alkyl-OC(O)R¹³³, —O—(C₁₋₆)alkyl-OS(O)₂R¹³⁴, NO₂, NR¹³⁵R¹³⁶, —NHC(O)R¹³⁷, substituted C₍₁₋₆₎alkyl, substituted C₍₁₋₆₎heteroalkyl, unsubstituted C₍₁₋₆₎alkyl, and unsubstituted C₍₁₋₆₎heteroalkyl.

In some embodiments of formula (1), each Q⁶ is independently selected from the group consisting of: halogen, —OR⁴¹, —O—(C₁₋₆)alkyl-NR⁴²R⁴³, —O—(C₁₋₆)alkyl-C(O)OR¹³⁸, —O—(C₁₋₆)alkyl-C(O)NHR¹³⁹, —O—(C₁₋₆)alkyl-OC(O)R¹⁴⁰, —O—(C₁₋₆)alkyl-OS(O)₂R¹⁴¹, NO₂, NR¹⁴²R¹⁴³, —NHC(O)R¹⁴⁴, substituted C(₁₋₆)alkyl, substituted C(₁₋₆)heteroalkyl, unsubstituted C(₁₋₆)alkyl, and unsubstituted C(₁₋₆)heteroalkyl.

In some embodiments of formula (1), each Q⁷ is independently selected from the group consisting of: halogen, —OR⁴⁴, —O—(C₁₋₆)alkyl-NR⁴⁵R⁴⁶, —O—(C₁₋₆)alkyl-C(O)OR¹⁴⁵, —O—(C₁₋₆)alkyl-C(O)NHR¹⁴⁶, —O—(C₁₋₆)alkyl-OC(O)R¹⁴⁷, —O—(C₁₋₆)alkyl-OS(O)₂R¹⁴⁸, NO₂, NR¹⁴⁹R¹⁵⁰, —NHC(O)R¹⁵¹, substituted C₍₁₋₆₎alkyl, substituted C₍₁₋₆₎heteroalkyl, unsubstituted C₍₁₋₆₎alkyl, and unsubstituted C₍₁₋₆₎heteroalkyl.

In some embodiments of formula (1), each Q⁸ is independently selected from the group consisting of: halogen, —OR⁴⁷, —O—(C₁₋₆)alkyl-NR⁴⁸R⁴⁹, —O—(C₁₋₆)alkyl-C(O)OR¹⁵², —O—(C₁₋₆)alkyl-C(O)NHR¹⁵³, —O—(C₁₋₆)alkyl-OC(O)R¹⁵⁴, —O—(C₁₋₆)alkyl-OS(O)₂R¹⁵⁵, NO₂, NR¹⁵⁶R¹⁵⁷, —NHC(O)R¹⁵⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl.

In some embodiments of formula (1), each Q⁹ is independently selected from the group consisting of: halogen, —OR¹⁵⁹, —O—(C₁₋₆)alkyl-NR¹⁶⁰R¹⁶¹, —O—(C₁₋₆)alkyl-C(O)OR¹⁶², —O—(C₁₋₆)alkyl-C(O)NHR¹⁶³, —O—(C₁₋₆)alkyl-OC(O)R¹⁶⁴, —O—(C₁₋₆)alkyl-OS(O)₂R¹⁶⁵, NO₂, NR¹⁶⁶R¹⁶⁷, —NHC(O)R¹⁶⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl.

In some embodiments of formula (1), each Q¹⁰ is independently selected from the group consisting of: halogen, —OR¹⁶⁹, —O—(C₁₋₆)alkyl-NR¹⁷⁰R¹⁷¹, —O—(C₁₋₆)alkyl-C(O)OR¹⁷², —O—(C₁₋₆)alkyl-C(O)NHR¹⁷³, —O—(C₁₋₆)alkyl-OC(O)R¹⁷⁴, —O—(C₁₋₆)alkyl-OS(O)₂R¹⁷⁵, NO₂, NR¹⁷⁶R¹⁷⁷, —NHC(O)R¹⁷⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl.

In some embodiments of formula (1), each Q¹¹ is independently selected from the group consisting of: halogen, —OR¹⁷⁹, —O—(C₁₋₆)alkyl-NR¹⁸⁰R¹⁸¹, —O—(C₁₋₆)alkyl-C(O)OR¹⁸², —O—(C₁₋₆)alkyl-C(O)NHR¹⁸³, —O—(C₁₋₆)alkyl-OC(O)R¹⁸⁴, —O—(C₁₋₆)alkyl-OS(O)₂R¹⁸⁵, NO₂, NR¹⁸⁶R¹⁸⁷, —NHC(O)R¹⁸⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl.

In some embodiments of formula (1), each Q¹² is independently selected from the group consisting of: halogen, —OR¹⁸⁹, —O—(C₁₋₆)alkyl-NR¹⁹⁰R¹⁹¹, —O—(C₁₋₆)alkyl-C(O)OR¹⁹², —O—(C₁₋₆)alkyl-C(O)NHR¹⁹³, —O—(C₁₋₆)alkyl-OC(O)R¹⁹⁴, —O—(C₁₋₆)alkyl-OS(O)₂R¹⁹⁵, NO₂, NR¹⁹⁶R¹⁹⁷, —NHC(O)R¹⁹⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl.

In some embodiments of formula (1), each Q¹³ is independently selected from the group consisting of: halogen, —OR¹⁹⁹, —O—(C₁₋₆)alkyl-NR²⁰⁰R²⁰¹, —O—(C₁₋₆)alkyl-C(O)OR²⁰², —O—(C₁₋₆)alkyl-C(O)NHR²⁰³, —O—(C₁₋₆)alkyl-OC(O)R²⁰⁴, —O—(C₁₋₆)alkyl-OS(O)₂R²⁰⁵, NO₂, NR²⁰⁶R²⁰⁷, —NHC(O)R²⁰⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl.

In some embodiments of formula (1) each R²⁶, R²⁷, R²⁸, R²⁹, R³⁰, R³¹, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R¹⁰⁰, R¹⁰⁴, R¹⁰⁵, R¹⁰⁷, R¹¹¹, R¹¹², R¹¹⁴, R¹¹⁵, R¹¹⁶, R¹¹⁷, R¹²¹, R¹²², R¹²⁴, R¹²⁸, R¹²⁹, R¹³¹, R¹³⁵, R¹³⁶, R¹³⁸, R¹⁴², R¹⁴³, R¹⁴⁵, R¹⁴⁹, R¹⁵⁰, R¹⁵², R¹⁵⁶, R¹⁵⁷, R¹⁵⁹, R¹⁶⁰, R¹⁶¹, R¹⁶², R¹⁶⁶, R¹⁶⁷, R¹⁶⁹, R¹⁷⁰, R¹⁷¹, R¹⁷², R¹⁷⁶, R¹⁷⁷, R¹⁷⁹, R¹⁸⁰, R¹⁸¹, R¹⁸², R¹⁸⁶, R¹⁸⁷, R¹⁸⁹, R¹⁹⁰, R¹⁹¹, R¹⁹², R¹⁹⁶, R¹⁹⁷, R¹⁹⁹, R²⁰⁰, R²⁰¹, R²⁰², R²⁰⁶ and R²⁰⁷ are independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and each pair: a) R²⁷ and R²⁸, b) R³⁰ and R³¹, c) R³⁶ and R³⁷, d) R³⁹ and R⁴⁰, e) R⁴² and R⁴³, f) R⁴⁵ and R⁴⁶, g) R⁴⁸ and R⁴⁹, h) R¹⁰⁴ and R¹⁰⁵, i) R¹¹¹ and R¹¹², j) R¹¹⁵ and R¹¹⁶, k) R¹²¹ and R¹²², l) R¹²⁸ and R¹²⁹, m) R¹³⁵ and R¹³⁶, n) R¹⁴² and R¹⁴³, o) R¹⁴⁹ and 150, p) R¹⁵⁶ and R¹⁵⁷, q) R¹⁶⁰ and R¹⁶¹, r) R¹⁶⁶ and R¹⁶⁷, s) R¹⁷⁰ and R¹⁷¹, t) R¹⁷⁶ and R¹⁷⁷, u) R¹⁸⁰ and R¹⁸¹, v) R¹⁸⁶ and R¹⁸⁷, w) R¹⁹⁰ and R¹⁹¹, x) R¹⁹⁶ and R¹⁹⁷, y) R²⁰⁰ and R²⁰¹, and z) R²⁰⁶ and R²⁰⁷ may alternately be and independently as a pair be a 3-7 membered substituted heterocarbocyclic ring or a 3-7 membered unsubstituted heterocarbocyclic ring.

In some embodiments of formula (1), R¹⁰¹, R¹⁰⁸, R¹¹⁸, R¹²⁵, R¹³², R¹³⁹, R¹⁴⁶, R¹⁵³, R¹⁶³, R¹⁷³, R¹⁸³, R¹⁹³ and R²⁰³ are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, unsubstituted C(₂₋₁₁)heteroaralkyl, substituted C(₁₋₆)alkyl-NR²⁰⁹R²¹⁰, unsubstituted C(₁₋₆)alkyl-NR²⁰⁹R²¹⁰, substituted C(₁₋₆)alkyl-N⁺R²¹¹R²¹²R²¹³, unsubstituted C(₁₋₆)alkyl-N⁺R²¹¹R²¹²R²¹³, substituted C(₁₋₆)alkyl-OR²¹⁴, unsubstituted C(₁₋₆)alkyl-OR²¹⁴,

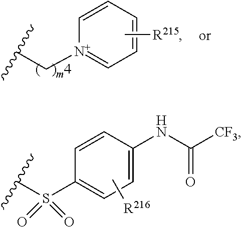

wherein m⁴ is 1, 2, 3, 4 or 5, R²⁰⁹, R²¹⁰, R²¹⁴, R²¹⁵ and R²¹⁶ are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and R²⁰⁹ and R²¹⁰, may alternately be and independently as a pair be a 3-7 membered substituted heterocarbocyclic ring or a 3-7 membered unsubstituted heterocarbocyclic ring, and R²¹¹, R²¹² and R²¹³ are each independently unsubstituted C(₁₋₁₁)alkyl, or substituted C(₁₋₁₁)alkyl.

In some embodiments of formula (1), R¹⁰¹ is selected from the group consisting of: unsubstituted C(₁₋₆)alkyl-NR²⁰⁹R²¹, unsubstituted C(₁₋₆)alkyl-N⁺R²¹¹R²¹²R²¹³, unsubstituted C(₁₋₆)alkyl-OR²¹⁴,

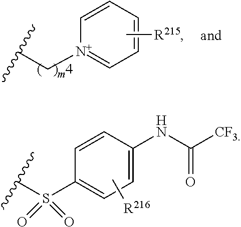

In some embodiments of formula (1), R¹⁰², R¹⁰³, R¹⁰⁶, R¹⁰⁹, R¹¹⁰, R¹¹³, R¹¹⁹, R¹²⁰, R¹²³, R¹²⁶, R¹²⁷, R¹³⁰, R¹³³, R¹³⁴, R¹³⁷, R¹⁴⁰, R¹⁴¹, R¹⁴⁴, R¹⁴⁷, R¹⁴⁸, R¹⁵¹, R¹⁵⁴, R¹⁵⁵, R¹⁵⁸, R¹⁶⁴, R¹⁶⁵, R¹⁶⁸, R¹⁷⁴, R¹⁷⁵, R¹⁷⁸, R¹⁸⁴, R¹⁸⁵, R¹⁸⁸, R¹⁹⁴, R¹⁹⁵, R¹⁹⁸, R²⁰⁴, R²⁰⁵ and R²⁰⁸ are each independently substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl;

Each one and/or every one of the embodiments set out above may be present in a compound of formula (1) independently or together with another embodiment. Some non-limiting examples of embodiments of formula (1) comprising more than one of the embodiments as set out above include the following.

In some embodiments of formula (1), G¹ is S and G⁴ is

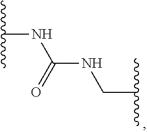 [SMILES: *CNC(=O)N*]

In some embodiments of formula (1), G¹ is NH, and G⁴ is a bond.

In some embodiments of formula (1), G¹⁴ is NH and G¹⁵ is CH.

In some embodiments of formula (1), G¹⁶ is CH and G¹⁷ is CH.

In some embodiments of formula (1), n is at least one 1 and Q² is selected from the group consisting of: halogen, NR¹¹¹R¹¹², NHC(O)R¹¹³, and substituted C(₁₋₆) alkyl.

In some of these embodiments, the substituted C₍₁₋₆₎ alkyl is a halogen substituted methyl group. In some of these embodiments, the halogen substituted methyl group is CF₃.

In some embodiments of formula (1), each of R¹, R², R³, R⁴, R⁵, R⁶, R¹², R¹³, R¹⁴, R¹⁷, R¹⁸, R¹⁹, R²⁴, and R²⁵ is independently selected from the group consisting of: H, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralky, and each of R²¹, R²², R²³, R⁶¹, R⁶² and R⁶³ is independently selected from the group consisting of: H, F, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl; and each pair: a) R² and R³, b) R⁵ and R⁶, c) R¹³ and R¹⁴, and d) R¹⁷ and R¹⁸ may alternately be and independently as a pair be a 3-7 membered substituted heterocarbocyclic ring or a 3-7 membered unsubstituted heterocarbocyclic ring; and R⁶⁰ is unsubstituted C(₁₋₁₁)alkyl, substituted C(₁₋₁₁)alkyl, unsubstituted C(₁₋₁₁)alkyl-NR⁶⁶R⁶⁷, substituted C(₁₋₁₁)alkyl-NR⁶⁶R⁶⁷, unsubstituted C(₁₋₁₁)alkyl-N⁺R⁶⁸R⁶⁹R⁷⁰, or substituted C(₁₋₁₁)alkyl-N⁺R⁶⁸R⁶⁹R⁷⁰, wherein R⁶⁶ and R⁶⁷ are each independently H, unsubstituted C(₁₋₁₁)alkyl or substituted C(₁₋₁₁)alkyl, and R⁶⁸, R⁶⁹ and R⁷⁰ are each independently unsubstituted C(₁₋₁₁)alkyl, or substituted C(₁₋₁₁)alkyl, and each of R¹⁵ and R¹⁶ is independently selected from the group consisting of: H, substituted C(₁₋₆)alkyl, substituted C(₁₋₁₁)aryl, substituted C(₁₋₁₁)heteroaryl, substituted C(₇₋₁₁)aralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl, or R¹⁵ and R¹⁶ may alternately be a 3-7 membered unsubstituted heterocarbocyclic ring; and

each of R⁶⁴ and R⁶⁵ is independently selected from the group consisting of: H, substituted C(₃₋₆)alkyl, substituted C(₁₋₁₁)aryl, substituted C(₁₋₁₁)heteroaryl, substituted C(₇₋₁₁)aralkyl, unsubstituted C(₂₋₁₁)alkyl, unsubstituted C(₁₋₁₁)aryl, unsubstituted C(₁₋₁₁)heteroaryl, and unsubstituted C(₈₋₁₁)aralky, or R⁶⁴ and R⁶⁵ may alternately be a 3-7 membered substituted heterocarbocyclic ring or a 3-7 membered unsubstituted heterocarbocyclic ring; and
each of R⁷ and R⁸ are either

I) independently selected from the group consisting of: H, substituted C(₁₋₆)alkyl, substituted C(₁₋₆)alkyl-NR⁵²R⁵³, unsubstituted C(₁₋₆)alkyl-NR⁵²R⁵³, substituted C(₁₋₆)alkyl-N⁺R⁷¹R⁷²R⁷³, unsubstituted C(₁₋₆)alkyl-N⁺R⁷¹R⁷²R⁷³, substituted C(₁₋₆)alkyl-OC(O)unsubstituted C(₁₋₆)alkyl-NR⁷⁴R⁷⁵, unsubstituted C(₁₋₆)alkyl-OC(O)unsubstituted C(₁₋₆)alkyl-NR⁷⁴R⁷⁵, substituted C(₁₋₆)alkyl-C(O)NHS(O)₂R⁷⁶, unsubstituted C(₁₋₆)alkyl-C(O)NHS(O)₂R⁷⁶, substituted C(₆₋₁₁)aryl, substituted C(₃₋₁₁)carbocyclic, substituted C(₄₋₇)heterocarbocycle, substituted C(₄₋₇)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₃₋₁₁)carbocyclic, unsubstituted C(₁₋₁₁)heterocarbocycle, unsubstituted C(₁₋₁₁)heteroaryl, unsubstituted C(₇₋₁₁)aralkyl, and unsubstituted C(₂₋₁₁)heteroaralkyl wherein each of R⁵², R⁵³, R⁷⁴ and R⁷⁵ is selected from the group consisting of: H, unsubstituted C(₁₋₆)alkyl, substituted C(₃₋₇)heterocycloalkyl, unsubstituted C(₃₋₇)heterocycloalkyl, substituted C(₁₋₆)alkyl, substituted C(₃₋₇)cycloalkyl and unsubstituted C(₃₋₇)cycloalkyl, or each pair: a) R⁵² and R⁵³, or (b) R⁷⁴ and R⁷⁵, together form a 3-7 membered substituted heterocarbocyclic ring or a 3-7 membered unsubstituted heterocarbocyclic ring, and wherein each of R⁷¹, R⁷², R⁷³ and R⁷⁶ is independently unsubstituted C(₁₋₁₁)alkyl, or substituted C(₁₋₁₁)alkyl, or

II) together form a 3-7 membered substituted heterocarbocyclic ring or a 3-7 membered unsubstituted heterocarbocyclic ring; and

R⁹ is selected from the group consisting of substituted C(₁₋₆)alkyl, substituted C(₁₋₆)alkyl-NR¹⁰R¹¹, unsubstituted C(₁₋₆)alkyl-NR¹⁰R¹¹, substituted C(₁₋₆)alkyl-OR²⁰, unsubstituted C(₁₋₆)alkyl-OR²⁰, and unsubstituted C(₄₋₆)alkyl wherein each of R¹⁰, R¹¹ and R²⁰ is independently selected from the group consisting of: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; R¹⁰ and R¹¹ may alternately as a pair be a 3-7 membered substituted heterocarbocyclic ring or a 3-7 membered unsubstituted heterocarbocyclic ring, or

G⁹ is

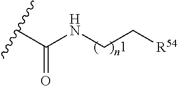

wherein n¹ is 1, 2, 3 or 4 and R⁵⁴ is

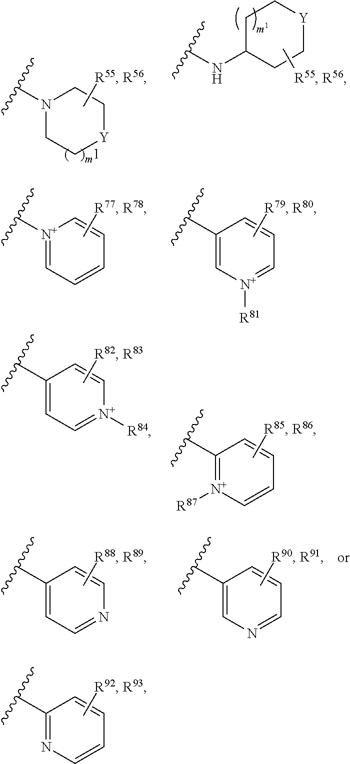

wherein m=0, 1 or 2, R⁵⁵ and R⁵⁶ are independently H, carbonyl (═O), Me, Ph, CO₂R⁹⁴, CO₂NH₂, C(₁₋₆)substituted alkyl or C(₁₋₆)unsubstituted alkyl, wherein R⁹⁴ is H, C(₁₋₆)unsubstituted alkyl or C(₁₋₆)substituted alkyl; and R⁷⁷, R⁷⁸, R⁷⁹, R⁸⁰, R⁸², R⁸³, R⁸⁵, R⁸⁶, R⁸⁸, R⁸⁹, R⁹⁰, R⁹¹, R⁹² and R⁹³ are each independently H, C(₁₋₆)substituted alkyl, C(₁₋₆)unsubstituted alkyl, substituted C(₁₋₆)heteroalkyl, unsubstituted C₍₁₋₆₎ heteroalkyl, OR⁹⁵, C(O)R⁹⁶, or NR⁹⁷R⁹⁸, wherein R⁹⁵ is H, C(₁₋₆)substituted alkyl, or C(₁₋₆)unsubstituted alkyl, R⁹⁶ is C(₁₋₆)substituted alkyl, or C(₁₋₆)unsubstituted alkyl, and R⁹⁷ and R⁹⁸ are each independently H, C(₁₋₆)substituted alkyl, or C(₁₋₆)unsubstituted alkyl, or each pair: a) R⁷⁷ and R⁷⁸, b) R⁷⁹ and R⁸⁰, c) R⁸² and R⁸³, d) R⁸⁵ and R⁸⁶, e) R⁸⁸ and R⁸⁹, f) R⁹⁰ and R⁹¹, or g) R⁹² and R⁹³ are attached to adjacent ring-forming C atoms, and together with the ring-forming C atoms, form a substituted C₆ aryl ring or an unsubstituted C₆ aryl ring; and
R⁸¹, R⁸⁴ and R⁸⁷ each independently is C(₁₋₆)substituted alkyl, or C(₁₋₆)unsubstituted alkyl; and
Y is CH₂, CHOH, CHO—CO—C(₁₋₆)unsubstituted alkyl, CHO—CO—C(₁₋₆)substituted alkyl, NCONH₂, N—C(₁₋₆)substituted alkyl, N—C(₁₋₆)unsubstituted alkyl, NH or N—C(O)OR⁹⁹ wherein R⁹⁹ is C(₁₋₆)unsubstituted alkyl, C(₁₋₆)substituted alkyl, C(₆₋₁₁)unsubstituted aralkyl or C(₆₋₁₁)substituted aralkyl.

In some embodiments of formula (1), each of R¹, R², R³, R⁴, R⁵, R⁶, R¹², R¹³, R¹⁴, R¹⁵, R¹⁶, R¹⁷, R¹⁸, R¹⁹, R²⁴, and R²⁵ is independently selected from the group consisting of: H, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl, and each of R²¹, R²², R²³, R⁶¹, R⁶² and R⁶³ is independently selected from the group consisting of: H, F, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl; and each of R⁶⁴ and R⁶⁵ is independently selected from the group consisting of: H, substituted C(₃₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl; and

each pair: a) R² and R³, b) R⁵ and R⁶, c) R¹³ and R¹⁴, d) R¹⁵ and R¹⁶, e) R¹⁷ and R¹⁸, and f) R⁶⁴ and R⁶⁵ may alternately be and independently as a pair be a 3-7 membered substituted heterocarbocyclic ring or a 3-7 membered unsubstituted heterocarbocyclic ring; and
R⁶⁰ is unsubstituted C(₁₋₁₁)alkyl, substituted C(₁₋₁₁)alkyl, unsubstituted C(₁₋₁₁)alkyl-NR⁶⁶R⁶⁷, substituted C(₁₋₁₁)alkyl-NR⁶⁶R⁶⁷, unsubstituted C(₁₋₁₁)alkyl-N⁺R⁶⁸R⁶⁹R⁷⁰, or substituted C(₁₋₁₁)alkyl-N⁺R⁶⁸R⁶⁹R⁷⁰, wherein R⁶⁶ and R⁶⁷ are each independently H, unsubstituted C(₁₋₁₁)alkyl or substituted C(₁₋₁₁)alkyl, and R⁶⁸, R⁶⁹ and R⁷⁰ are each independently unsubstituted C(₁₋₁₁)alkyl, or substituted C(₁₋₁₁)alkyl, and
each of R⁷ and R⁸ are either

I) independently selected from the group consisting of: H, substituted C(₁₋₆)alkyl, substituted C(₁₋₆)alkyl-NR⁵²R⁵³, unsubstituted C(₁₋₆)alkyl-NR⁵²R⁵³, substituted C(₁₋₆)alkyl-N⁺R⁷¹R⁷²R⁷³, unsubstituted C(₁₋₆)alkyl-N⁺R⁷¹R⁷²R⁷³, substituted C(₁₋₆)alkyl-OC(O)unsubstituted C(₁₋₆)alkyl-NR⁷⁴R⁷⁵, unsubstituted C(₁₋₆)alkyl-OC(O)unsubstituted C(₁₋₆)alkyl-NR⁷⁴R⁷⁵, substituted C(₁₋₆)alkyl-C(O)NHS(O)₂R⁷⁶, unsubstituted C(₁₋₆)alkyl-C(O)NHS(O)₂R⁷⁶, substituted C(₆₋₁₁)aryl, substituted C(₃₋₁₁)carbocyclic, substituted C(₄₋₇)heterocarbocycle, substituted C(₄₋₇)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₃₋₁₁)carbocyclic, unsubstituted C(₁₋₁₁)heterocarbocycle, unsubstituted C(₁₋₁₁)heteroaryl, unsubstituted C(₇₋₁₁)aralkyl, and unsubstituted C(₂₋₁₁)heteroaralkyl wherein each of R⁵², R⁵³, R⁷⁴ and R⁷⁵ is selected from the group consisting of: H, unsubstituted C(₁₋₆)alkyl, substituted C(₃₋₇)heterocycloalkyl, unsubstituted C(₃₋₇)heterocycloalkyl, substituted C(₁₋₆)alkyl, substituted C(₃₋₇)cycloalkyl and unsubstituted C(₃₋₇)cycloalkyl, or each pair: a) R⁵² and R⁵³, or (b) R⁷⁴ and R⁷⁵, together form a 3-7 membered substituted heterocarbocyclic ring or a 3-7 membered unsubstituted heterocarbocyclic ring, and wherein each of R⁷¹, R⁷², R⁷³ and R⁷⁶ is independently unsubstituted C(₁₋₁₁)alkyl, or substituted C(₁₋₁₁)alkyl, or

II) together form a 3-7 membered substituted heterocarbocyclic ring or a 3-7 membered unsubstituted heterocarbocyclic ring; and

R⁹ is selected from the group consisting of substituted C(₁₋₆)alkyl, substituted C(₁₋₆)alkyl-NR¹⁰R¹¹, unsubstituted C(₁₋₆)alkyl-NR¹⁰R¹¹, substituted C(₁₋₆)alkyl-OR²⁰, unsubstituted C(₁₋₆)alkyl-OR²⁰, and unsubstituted C(₁₋₆)alkyl wherein each of R¹⁰, R¹¹ and R²⁰ is independently selected from the group consisting of: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; R¹⁰ and R¹¹ may alternately as a pair be a 3-7 membered substituted heterocarbocyclic ring or a 3-7 membered unsubstituted heterocarbocyclic ring, or

G⁹ is

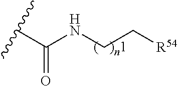

wherein n¹ is 1, 2, 3 or 4 and R⁵⁴ is

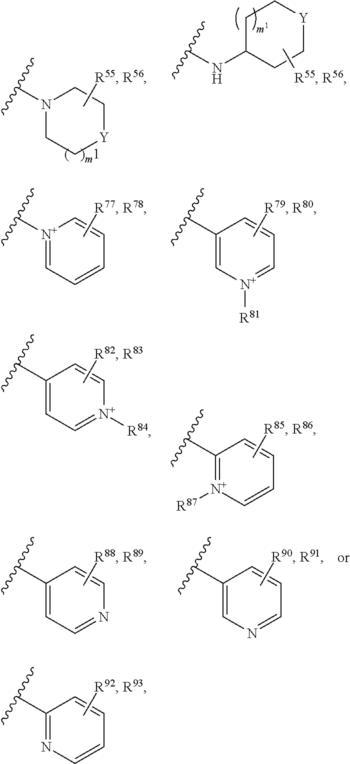

wherein m¹=0, 1 or 2, R⁵⁵ and R⁵⁶ are independently H, carbonyl (═O), Me, Ph, CO₂R⁹⁴, CO₂NH₂, C(₁₋₆)substituted alkyl or C(₁₋₆)unsubstituted alkyl, wherein R⁹⁴ is H, C(₁₋₆)unsubstituted alkyl or C(₁₋₆)substituted alkyl; and R⁷⁷, R⁷⁸, R⁷⁹, R⁸⁰, R⁸², R⁸³, R⁸⁵, R⁸⁶, R⁸⁸, R⁸⁹, R⁹⁰, R⁹¹, R⁹² and R⁹³ are each independently H, C(₁₋₆)substituted alkyl, C(₁₋₆)unsubstituted alkyl, substituted C(₁₋₆)heteroalkyl, unsubstituted C₍₁₋₆₎ heteroalkyl, OR⁹⁵, C(O)R⁹⁶, or NR⁹⁷R⁹⁸, wherein R⁹⁵ is H, C(₁₋₆)substituted alkyl, or C(₁₋₆)unsubstituted alkyl, R⁹⁶ is C(₁₋₆)substituted alkyl, or C(₁₋₆)unsubstituted alkyl, and R⁹⁷ and R⁹⁸ are each independently H, C(₁₋₆)substituted alkyl, or C(₁₋₆)unsubstituted alkyl, or each pair: a) R⁷⁷ and R⁷⁸, b) R⁷⁹ and R⁸⁰, c) R⁸² and R⁸³, d) R⁸⁵ and R⁸⁶, e) R⁸⁸ and R⁸⁹, f) R⁹⁰ and R⁹¹, or g) R⁹² and R⁹³ are attached to adjacent ring-forming C atoms, and together with the ring-forming C atoms, form a substituted C₆ aryl ring or an unsubstituted C₆ aryl ring; and
R⁸¹, R⁸⁴ and R⁸⁷ each independently is C(₁₋₆)substituted alkyl, or C(₁₋₆)unsubstituted alkyl; and
Y is CH₂, CHOH, CHO—CO—C(₁₋₆)unsubstituted alkyl, CHO—CO—C(₁₋₆)substituted alkyl, NCONH₂, N—C(₁₋₆)substituted alkyl, N—C(₁₋₆)unsubstituted alkyl, NH or N—C(O)OR⁹⁹, wherein R⁹⁹ is C(₁₋₆)unsubstituted alkyl, C(₁₋₆)substituted alkyl, C(₆₋₁₁)unsubstituted aralkyl or C(₆₋₁₁)substituted aralkyl.

In all of embodiments of formula (1), the following two criteria ((ii) and (iii)) are met:

(ii) when G³ is N, CH, or CG⁹ where G⁹ is C(O)OR⁹ and R⁹ is unsubstituted C₍₁₋₆₎ alkyl, G⁴ is other than

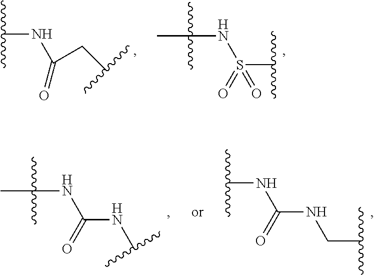 [SMILES: *CC(=O)N* ; *NS(*)(=O)=O ; *NC(=O)N* ; *CNC(=O)N*]

### and G⁵ is

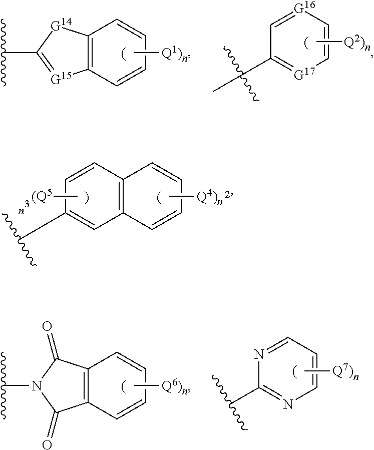

or a 5-membered heteroaryl optionally substituted with (Q⁸)ₙ and containing 1 or 2 heteroatoms each heteroatom independently selected from N, O and S, then n is at least 1 or n²+n³ is at least 1, and
(a) when n is 1 or n²+n³=1, then Q¹, Q², Q⁴, Q⁵, Q⁶, Q⁷ or Q⁸ is independently selected from the group consisting of —OR²⁶, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R¹⁰², —O—(C₁₋₆)alkyl-OS(O)₂R^{103′}, NR^{104′}R^{105′}, and —NHC(O)R^{106′}, wherein R^{26′} is independently selected from the group consisting of substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₂₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl;
   each R^{27′}, R^{28′}, R^{100′}, R^{104′} and R^{105′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; or each pair: a) R^{27′} and R^{28′}, or b) R^{104′} and R^{105′} may alternately be and independently as a pair be a 3-7 membered substituted heterocarbocyclic ring or a 3-7 membered unsubstituted heterocarbocyclic ring;

R^{101′} is H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, unsubstituted C(₂₋₁₁)heteroaralkyl, substituted C(₁₋₆)alkyl-NR^{209′}R^{210′}, unsubstituted C(₁₋₆)alkyl-NR^{209′}R²¹⁰ substituted C(₁₋₆)alkyl-N⁺R^{211′}R^{212′}R^{213′}, unsubstituted C(₁₋₆)alkyl-N⁺R^{211′}R^{212′}R^{213′}, substituted C(₁₋₆)alkyl-OR^{214′}, unsubstituted C(₁₋₆)alkyl-OR^{214′},

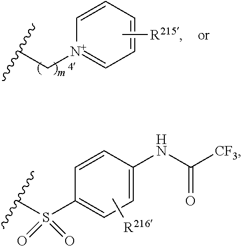

wherein m⁴ is 1, 2, 3, 4 or 5, R^{209′}, R^{210′}, R^{214′}, R^{215′} and R^{216′} are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and R^{209′} and R^{210′}, may alternately be and independently as a pair be a 3-7 membered substituted heterocarbocyclic ring or a 3-7 membered unsubstituted heterocarbocyclic ring, and R^{211′}, R^{212′} and R^{213′} are each independently unsubstituted C(₁₋₁₁)alkyl, or substituted C(₁₋₁₁)alkyl; and

R^{102′}, R^{103′}, and R^{10′} are each independently substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and
(b) when n is at least 2 or n²+n³ is at least 2, then a first Q¹, Q², Q⁴, Q⁵, Q⁶, Q⁷ or Q⁸ is independently selected from the group consisting of —OR^{26′}, —O—(CO₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NR^{104′}R^{105′}, and —NHC(O)R^{106′},
   wherein each of R^{26′}, R^{27′}, R^{28′}, R^{100′}, R^{101′}, R^{102′}, R^{103′}, R^{104′}, R^{105′}, and R^{106′} is as defined above; and

the remaining Q¹, Q², Q⁴, Q⁵, Q⁶, Q⁷ or Q⁸ are each independently selected from the group consisting of halogen, —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, NR^{104′}R^{105′}, —NHC(O)R^{106′}, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
wherein each R^{26′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and

each of R^{27′}, R²⁸, R^{100′}, R^{101′}, R^{102′}, R^{103′}, R^{104′}, R^{105′}, and R^{106′} is as defined above.
(iii) When G³ is N, CH, or CG⁹ where G⁹ is C(O)OR⁹ and R⁹ is unsubstituted C₍₁₋₆₎ alkyl, G⁴ is other than

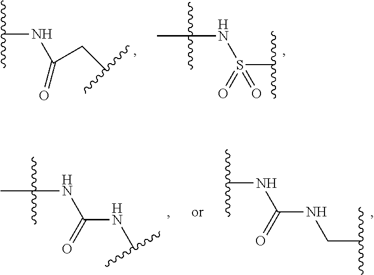 [SMILES: *CC(=O)N* ; *NS(*)(=O)=O ; *NC(=O)N* ; *CNC(=O)N*]

### and G⁵ is

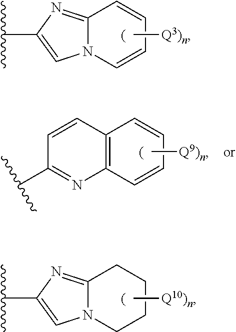

then n is at least 1 wherein each of Q³, Q⁹ and Q¹⁰ is as defined above.

In many of the embodiments of formula (1), the following seven criteria ((ii), (iii), (iv), (v), (vi), (vii) and (viii)) are met:
(ii) When G³ is N, CH, or CG⁹ where G⁹ is C(O)OR⁹ and R⁹ is unsubstituted C₍₄₋₆₎ alkyl, G⁴ is other than

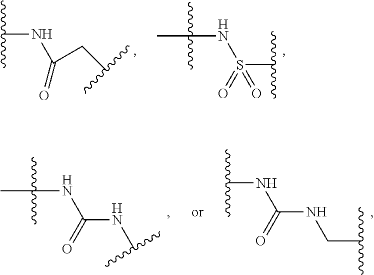 [SMILES: *CC(=O)N* ; *NS(*)(=O)=O ; *NC(=O)N* ; *CNC(=O)N*]

### and G⁵ is

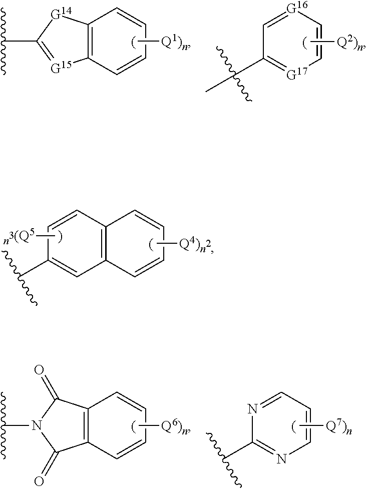

or a 5-membered heteroaryl optionally substituted with (Q⁸)ₙ and containing 1 or 2 heteroatoms each heteroatom independently selected from N, O and S, then n is at least 1 or n²+n³ is at least 1, and
(a) when n is 1 or n²+n³=1, then Q¹, Q², Q⁴, Q⁵, Q⁶, Q⁷ or Q⁸ is independently selected from the group consisting of —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, and —NHC(O)R^{106′}, wherein R^{26′} is independently selected from the group consisting of substituted C(₁₋₆)alkyl, substituted C(₇₋₁₁)aralkyl, substituted C(₂₋₁₁)heteroaralkyl, unsubstituted C(₅₋₁₁)alkyl, unsubstituted C(₇₋₁₁)aralkyl, and unsubstituted C(₂₋₁₁)heteroaralkyl; each of R^{27′}, R^{28′}, and R^{100′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; or R^{27′} and R^{28′} may alternately as a pair be a 3-7 membered substituted heterocarbocyclic ring or a 3-7 membered unsubstituted heterocarbocyclic ring;

R^{101′} is H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, unsubstituted C(₂₋₁₁)heteroaralkyl, substituted C(₁₋₆)alkyl-NR^{209′}R^{210′}, unsubstituted C(₁₋₆)alkyl-NR^{209′}R^{210′}, substituted C(₁₋₆)alkyl-N⁺R^{211′}R^{212′}R^{213′}, unsubstituted C(₁₋₆)alkyl-N⁺R^{211′}R^{212′}R^{213′}, substituted C(₁₋₆)alkyl-OR²¹⁴, unsubstituted C(₁₋₆)alkyl-OR^{214′},

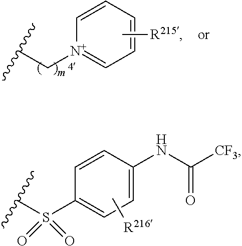

wherein m^{4′} is 1, 2, 3, 4 or 5, R^{209′}, R^{210′}, R^{214′}, R^{215′} and R^{216′} are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and R^{209′} and R^{210′}, may alternately be and independently as a pair be a 3-7 membered substituted heterocarbocyclic ring or a 3-7 membered unsubstituted heterocarbocyclic ring, and R^{211′}, R^{212′} and R^{213′} are each independently unsubstituted C(₁₋₁₁)alkyl, or substituted C(₁₋₁₁)alkyl; and

R^{102′} and R^{103′} are each independently substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, or unsubstituted C(₂₋₁₁)heteroaralkyl; and

R^{106′} is substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₂₋₁₁)alkyl, unsubstituted C(₆₋₁₁)aryl, unsubstituted C(₁₋₁₁)heteroaryl, unsubstituted C(₇₋₁₁)aralkyl, or unsubstituted C(₂₋₁₁)heteroaralkyl; and
(b) when n is at least 2 or n²+n³ is at least 2, then a first Q¹, Q², Q⁴, Q⁵, Q⁶, Q⁷ or Q⁸ is independently selected from the group consisting of —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, and —NHC(O)R^{106′},
   wherein each of R^{26′}, R^{27′}, R^{28′}, R^{100′}, R^{101′}, R^{102′}, R^{103′}, and R^{106′} is as defined above; and

the remaining Q¹, Q², Q⁴, Q⁵, Q⁶, Q⁷ or Q⁸ are each independently selected from the group consisting of halogen, —OR^{26′}, —O—(C₁₋₆)alkyl-NR^{27′}R²⁸, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, NR^{104′}R^{105′}, —NHC(O)R^{106′}, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;

wherein each R^{26′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl;

each of R^{104′} and R^{105′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; or R^{104′} and R^{105′} may alternately as a pair be a 3-7 membered substituted heterocarbocyclic ring or a 3-7 membered unsubstituted heterocarbocyclic ring;
each R^{106′} is substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, or unsubstituted C(₂₋₁₁)heteroaralkyl; and

each of R^{27′}, R^{28′}, R^{100′}, R^{101′}, R^{102′}, and R^{103′} is as defined above.

(iii) When G³ is N, CH, or CG⁹ where G⁹ is C(O)OR⁹ and R⁹ is unsubstituted C₍₄₋₆₎ alkyl, G⁴ is other than

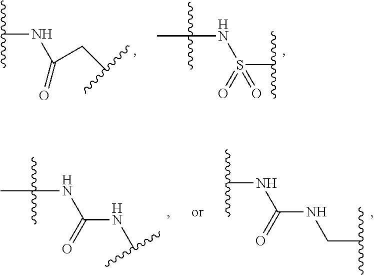 [SMILES: *CC(=O)N* ; *NS(*)(=O)=O ; *NC(=O)N* ; *CNC(=O)N*]

### and G⁵ is

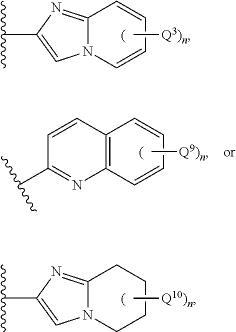

then at least one of G⁶, G⁷, and G⁸ is not H; n is at least 1; and each of Q³, Q⁹ or Q¹⁰ is independently selected from the group consisting of halogen, —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, —NHC(O)R^{106′}, substituted C₍₁₋₆₎alkyl, substituted C₍₁₋₆₎heteroalkyl, unsubstituted C₍₂₋₆₎alkyl, and unsubstituted C₍₁₋₆₎heteroalkyl.

(iv) When G³ is N or CH, and G⁵ is,

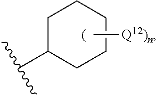

then at least one of G⁶, G⁷, and G⁸ is not H; n is at least 1; and each Q¹² is independently selected from the group consisting of halogen, —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, —NHC(O)R^{106′}, substituted C₍₁₋₆₎alkyl, substituted C₍₁₋₆₎heteroalkyl, unsubstituted C₍₁₋₆₎alkyl, and unsubstituted C₍₁₋₆₎heteroalkyl.

(v) When G³ is N or CH, and G⁴ is

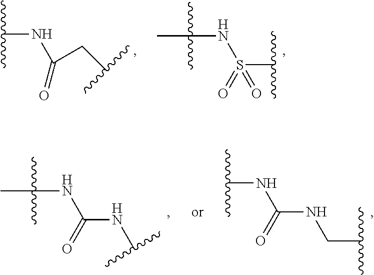 [SMILES: *CC(=O)N* ; *NS(*)(=O)=O ; *NC(=O)N* ; *CNC(=O)N*]

### and G⁵ is: (a)

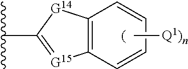

where G¹⁴ is CH₂ and G¹⁵ is N, or G¹⁴ is NH and G¹⁵ is CH, or G¹⁴ is S and G¹⁵ is CH; (b)

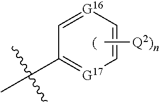

where G¹⁶ is N and G¹⁷ is N; or (c)

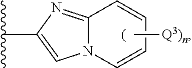

then at least one of G⁶, G⁷, and G⁸ is not H, and n is at least 1.

(vi) When G³ is N or CH, and G⁴ is

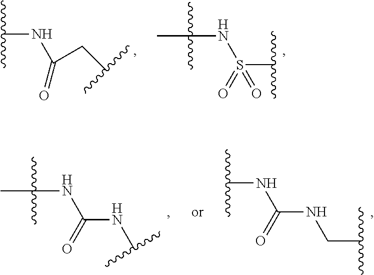 [SMILES: *CC(=O)N* ; *NS(*)(=O)=O ; *NC(=O)N* ; *CNC(=O)N*]

### and G⁵ is: (a)

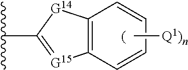

where G¹⁴ is NH and G¹⁵ is N; (b)

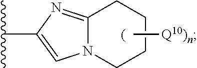

(c)

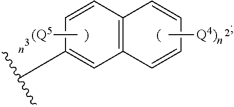

or (d)

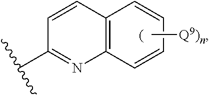

then at least one of G⁶, G⁷ and G⁸ is not H, and each of G⁶ and G⁷ is independently H, halogen, CF₃, NO₂, substituted (C₁₋₁₁)alkyl, unsubstituted (C₃₋₁₁)alkyl, substituted (C₁₋₁₁)alkoxyl, unsubstituted (C₁₋₁₁) alkoxyl, substituted (C₆₋₁₁)aryloxy, unsubstituted (C₆₋₁₁)aryloxy, C(O)OR⁵⁰, or

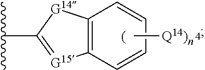

n is at least 1 or n²+n³ is at least 1; and each of Q¹, Q⁴, Q⁵, Q⁹, Q¹⁰ and Q¹² is independently selected from the group consisting of halogen, —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, —NHC(O)R^{106′}, substituted C₍₁₋₆₎alkyl, and unsubstituted C₍₂₋₆₎alkyl;
R^{106′} is substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₂₋₁₁)alkyl, unsubstituted C(₆₋₁₁)aryl, unsubstituted C(₁₋₁₁)heteroaryl, unsubstituted C(₇₋₁₁)aralkyl, or unsubstituted C(₂₋₁₁)heteroaralkyl; and
each of R^{26′}, R^{27′}, R^{28′}, R^{100′}, R^{101′}, R^{102′}, and R^{103′} is as defined above.

(vii) When G³ is N or CH, and G⁴ is

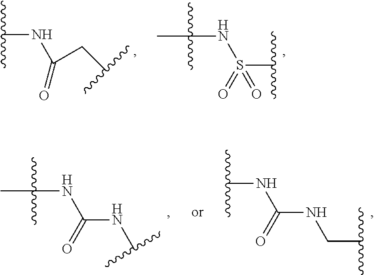 [SMILES: *CC(=O)N* ; *NS(*)(=O)=O ; *NC(=O)N* ; *CNC(=O)N*]

### and G⁵ is: (a)

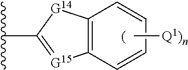

where G¹⁴ is S and G¹⁵ is N; (b)

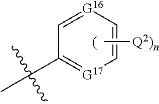

where G¹⁶ is CH and G¹⁷ is N, or G¹⁶ is N and G¹⁷ is CH, or G¹⁶ is CH and G¹⁷ is CH; (c)

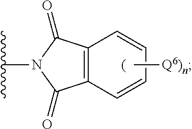

or (d) a 5-membered heteroaryl optionally substituted with (Q⁸)ₙ and containing 1 or 2 heteroatoms each heteroatom independently selected from N, O and S, then at least one of G⁶, G⁷ and G⁸ is not H, and each of G⁶ and G⁷ is independently H, halogen, CF₃, NO₂, substituted (C₁₋₁₁)alkyl, unsubstituted (C₃₋₁₁)alkyl, substituted (C₁₋₁₁)alkoxyl, unsubstituted (C₁₋₁₁) alkoxyl, substituted (C₆₋₁₁)aryloxy, unsubstituted (C₆₋₁₁)aryloxy, C(O)OR⁵⁰, or

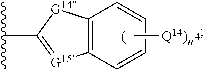

and n is at least 1; and
(a) when n is 1, then each of Q¹, Q², Q⁶, or Q⁸ is independently selected from the group consisting of —OR^{26′}, —O—(C₁₋₆)alkyl-NR^{27′}R²⁸, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, and —NHC(O)R^{106′},
   wherein each of R^{26′}, R^{27′}, R^{28′}, R^{100′}, R^{101′}, R^{102′}, R^{103′} and R^{106′} is as defined above; and
(b) when n is at least 2, then a first Q¹, Q², Q⁶, or Q⁸ is independently selected from the group consisting of —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, and —NHC(O)R^{106′},
   wherein each of R^{26′}, R^{27′}, R^{28′}, R^{100′} R^{101′}, R^{102′}, R^{103′}, and R^{106′} is as defined above; and

the remaining Q¹, Q², Q⁶, or Q⁸ are each independently selected from the group consisting of halogen, —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, NR^{104′}R^{105′}, —NHC(O)R^{106′}, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;

wherein each R^{26′}, R^{27′}, R^{28′}, R^{100′}, R^{101′}, R^{102′}, R^{103′}, R^{104′}, R^{105′}, and R^{106′} is as defined above.

(viii) When G³ is CG⁹ and G⁹ is: (a) substituted (C₁₋₆) alkyl-NH₂; (b) unsubstituted (C₁₋₆) alkyl-NH₂; (c) substituted (C₁₋₆) alkyl-NR⁶⁴R⁶⁵ or unsubstituted (C₁₋₆) alkyl-NR⁶⁴R⁶⁵ where R⁶⁴ and R⁶⁵ as a pair are a 3-7 membered substituted heterocarbocyclic ring or a 3-7 membered unsubstituted heterocarbocyclic ring; (d) substituted (C₆₋₁₁) aryl; (e) substituted (C₁₋₁₁) heteroaryl or unsubstituted (C₁₋₁₁) heteroaryl; (f) substituted (C₆₋₁₁) arylcarbonyl or unsubstituted (C₆₋₁₁) arylcarbonyl; (g) substituted (C₁₋₁₁) heteroarylcarbonyl or unsubstituted (C₁₋₁₁) heteroarylcarbonyl; (h) —CO-substituted-carbocycle or —CO-unsubstituted-carbocycle; (i) —CO-substituted-heterocarbocycle or —CO-unsubstituted-heterocarbocycle; (j) —C(O)NR⁷R⁸ where each of R⁷ and R⁸ is CH₃; (k) —C(O)NR⁷R⁸ where R⁷ is H and R⁸ is unsubstituted C₆ aryl or unsubstituted C₄ cycloalkyl; (l) —C(O)C(O)NR¹³R¹⁴ where each of R¹³ and R¹⁴ is CH₃; (m) —C(O)C(O)NR¹³R¹⁴ where each of R¹³ and R¹⁴ is

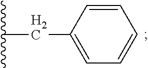 [SMILES: *Cc1ccccc1]

(n) —NR¹⁵R¹⁶ where only one of R¹⁵ and R¹⁶ is unsubstituted C₆ aryl; or (o) —NR¹⁵R¹⁶ where R¹⁵ and R¹⁶ as a pair are a 3-7 membered unsubstituted heterocarbocyclic ring, then at least one of G⁶, G⁷ and G⁸ is not H.

Illustrative embodiments of the present invention provide a compound which is a dimer comprising two of the same or different compounds of formula (1), wherein the first compound of formula (1) and the second compound of formula (1) are covalently linked through a covalent linkage of a moiety of G⁹ of the first compound of formula (1) and a moiety of G⁹ of the second compound of formula (1).

In some embodiments, the dimer has the structure of

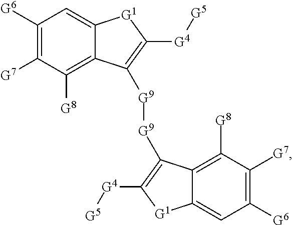

wherein:
each G¹ of the first and second compounds is the same or different and is as defined anywhere herein; each G⁴ of the first and second compounds is the same or different and is as defined anywhere herein; each G⁵ of the first and second compounds is the same or different and is as defined anywhere herein; each G⁶ of the first and second compounds is the same or different and is as defined anywhere herein; each G⁷ of the first and second compounds is the same or different and is as defined anywhere herein; each G⁸ of the first and second compounds is the same or different and is as defined anywhere herein; and each G⁹ of the first and second compounds is the same or different and is as defined anywhere herein wherein a moiety of G⁹ of the first compound is linked to a moiety of G⁹ of the second compound through a covalent linkage.

In some embodiments, the covalently linked G⁹ groups of the first and second compounds of the dimer have the structure selected from the group consisting of:

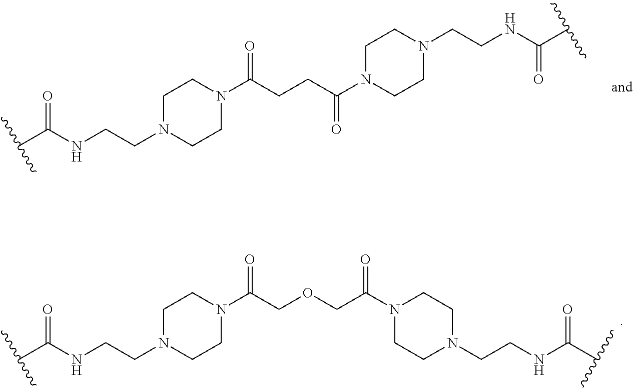 [SMILES: *C(=O)NCCN1CCN(C(=O)CCC(=O)N2CCN(CCNC(*)=O)CC2)CC1 ; *C(=O)NCCN1CCN(C(=O)COCC(=O)N2CCN(CCNC(*)=O)CC2)CC1]

In some embodiments, the present invention provides a compound selected from the group consisting of:

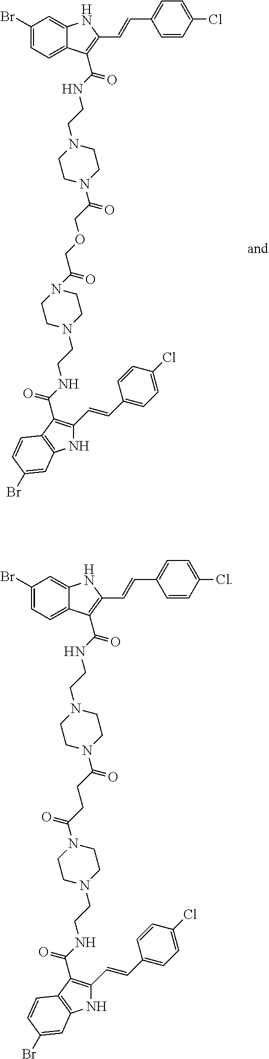 [SMILES: O=C(NCCN1CCN(C(=O)COCC(=O)N2CCN(CCNC(=O)c3c(/C=C/c4ccc(Cl)cc4)[nH]c4cc(Br)ccc34)CC2)CC1)c1c(/C=C/c2ccc(Cl)cc2)[nH]c2cc(Br)ccc12 ; O=C(NCCN1CCN(C(=O)CCC(=O)N2CCN(CCNC(=O)c3c(/C=C/c4ccc(Cl)cc4)[nH]c4cc(Br)ccc34)CC2)CC1)c1c(/C=C/c2ccc(Cl)cc2)[nH]c2cc(Br)ccc12]

With respect to the embodiments in which G², G³ and G⁴ together form a ring moiety selected from the group consisting of:

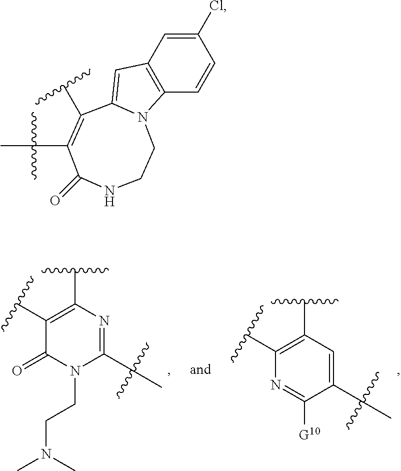

in each case, regarding the 2 adjacent points of attachment connected by a double bond to the left of the moiety as set out herein, the lower point of attachment is G³ and G³ attaches to a carbon atom as set out in general formula (1) depicted herein. The higher point of attachment is G² and G² attaches to G¹ as set out in general formula (1) depicted herein. For those moieties in which there is a third point of attachment, the third point of attachment attaches to G⁵ as set out herein.

In some embodiments of the present invention, each Q¹ is independently selected from the group consisting of: H, halogen, —OR²⁶, and —O—(C₁₋₆)alkyl-NR²⁷R²⁸;

each Q² is independently selected from the group consisting of: H, halogen, —OR²⁹, and —O—(C₁₋₆)alkyl-NR³⁰R³¹;

each Q⁴ is independently selected from the group consisting of: H, halogen, —OR³⁵, and —O—(C₁₋₆)alkyl-NR³⁶R³⁷;

each Q⁵ is independently selected from the group consisting of: H, halogen, —OR³⁸, and —O—(C₁₋₆)alkyl-NR³⁹R⁴⁰;

each Q⁶ is independently selected from the group consisting of: H, halogen, —OR⁴¹, and —O—(C₁₋₆)alkyl-NR⁴²R⁴³;

each Q⁷ is independently selected from the group consisting of: H, halogen, —OR⁴⁴, and —O—(C₁₋₆)alkyl-NR⁴⁵R⁴⁶;

each Q⁸ is independently selected from the group consisting of: H, halogen, —OR⁴⁷, and —O—(C₁₋₆)alkyl-NR⁴⁸R⁴⁹;

In some embodiments of the present invention G⁵ is selected from the group consisting of:

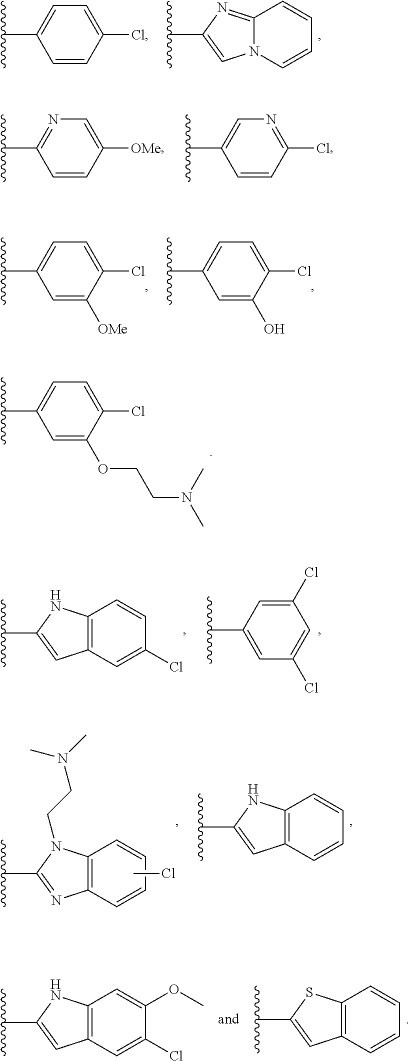

In some embodiments of the present invention G⁴ is selected from the group consisting of:

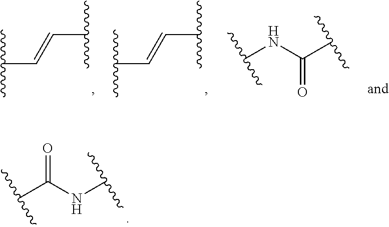

In some embodiments of the present invention G⁴ and G⁵, when considered together, are selected from the group consisting of:

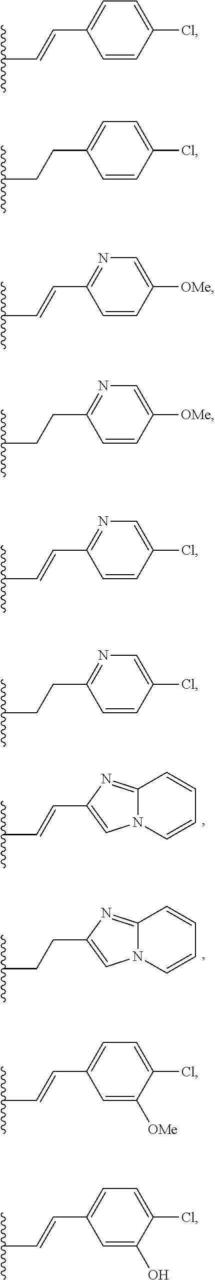

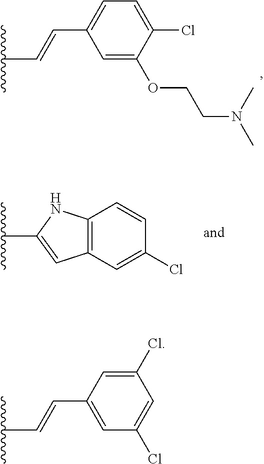 [SMILES: */C=C/c1ccc(Cl)c(OCCN(C)C)c1 ; *c1cc2cc(Cl)ccc2[nH]1 ; */C=C/c1cc(Cl)cc(Cl)c1]

In some embodiments of the present invention G⁴ and G⁵, when considered together, are selected from the group consisting of:

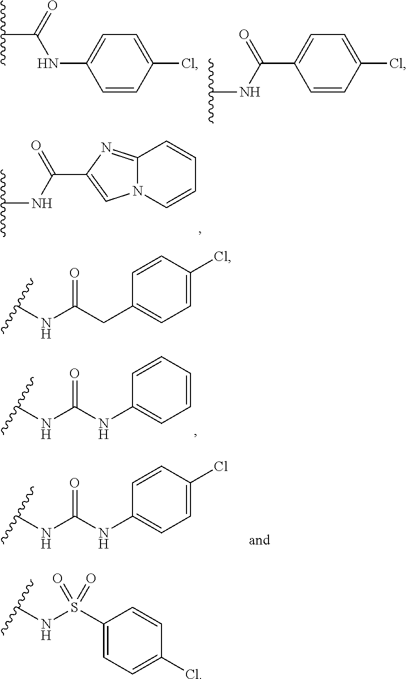

In some embodiments of the present invention G⁹ is selected from the group consisting of:

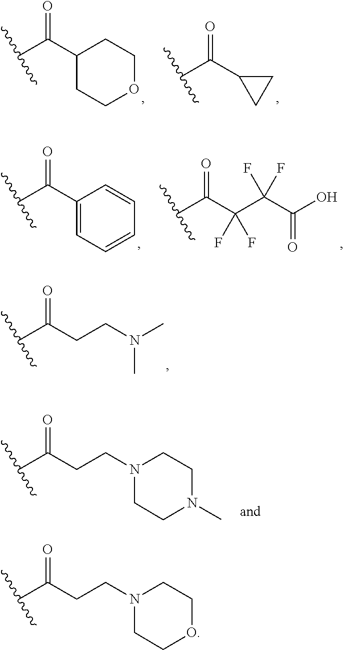

In some embodiments of the present invention G⁹ is selected from the group consisting of:

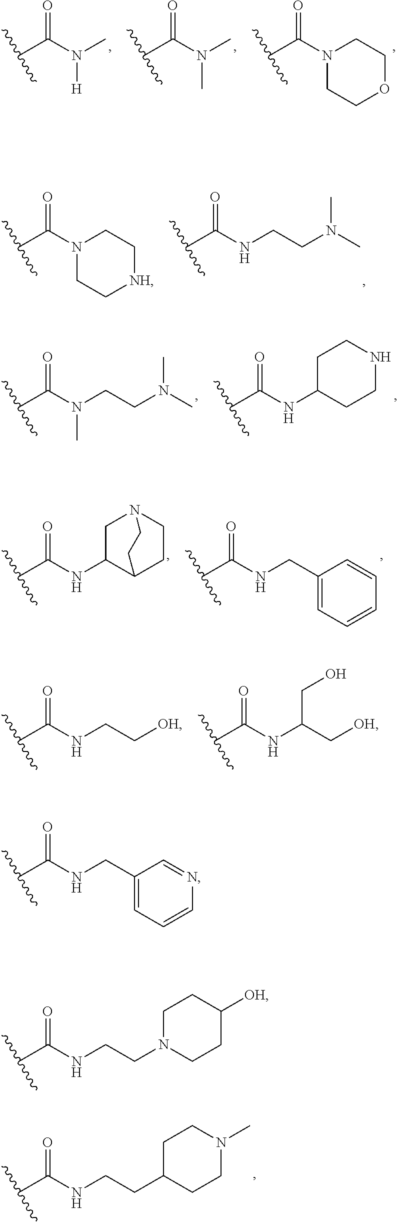

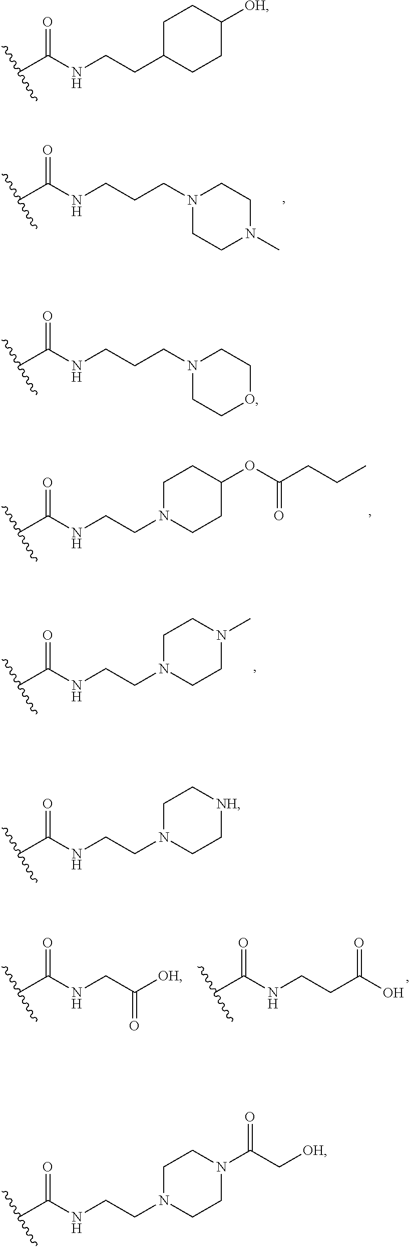

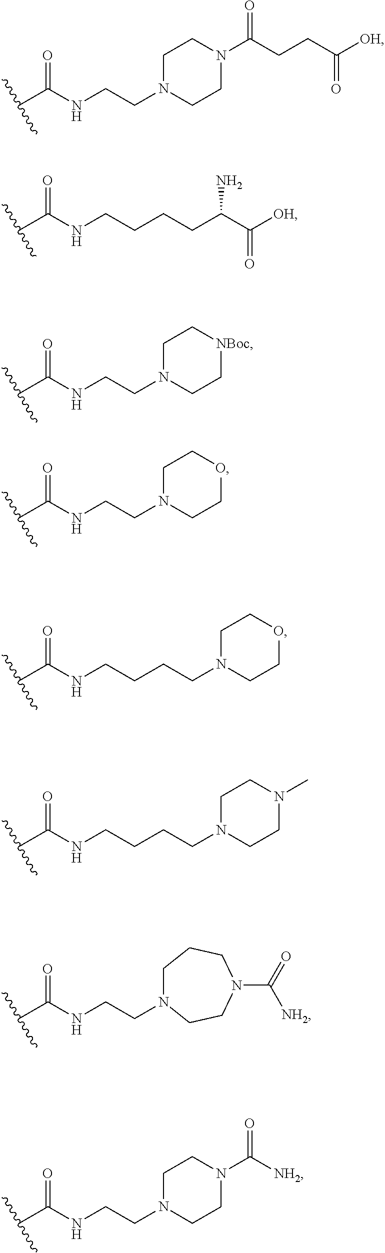

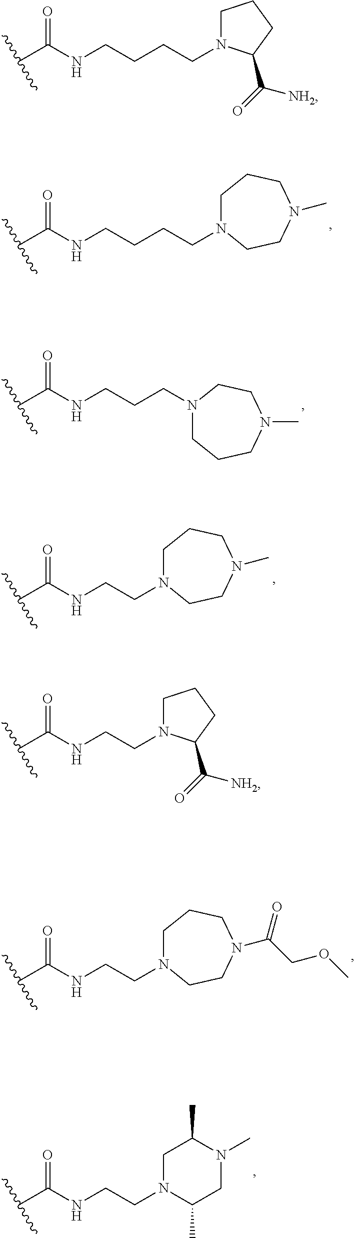

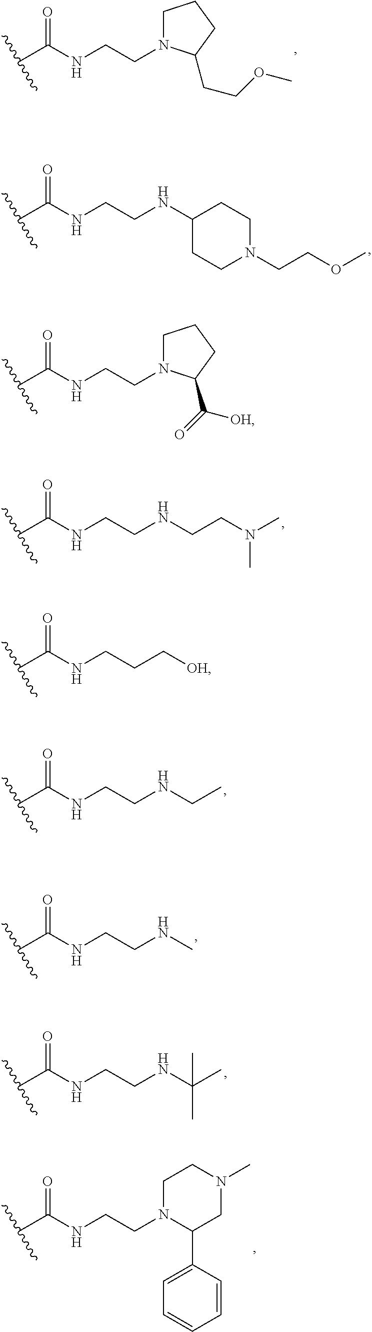

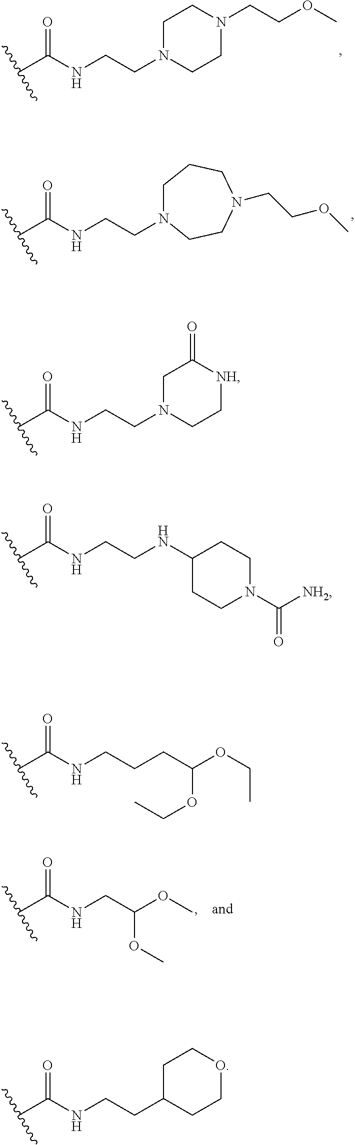

In some embodiments of the present invention G⁹ is

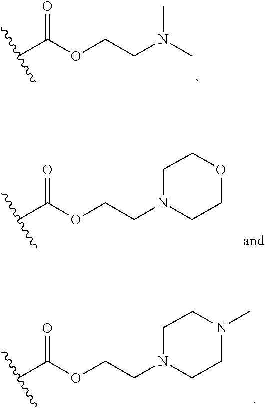

In some embodiments of the present invention G⁹ is selected from the group consisting of:

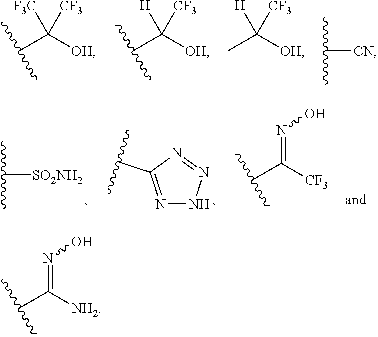

In some embodiments of the invention, G⁹ is selected from the group consisting of:

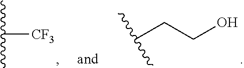

In some embodiments of the invention, G⁹ is selected from the group consisting of:

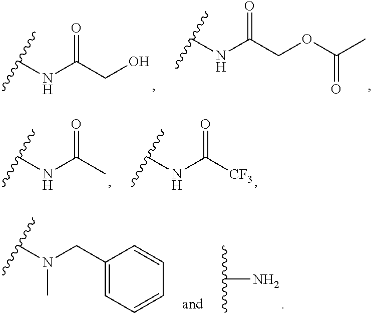

In some embodiments of the invention, G⁹ is selected from the group consisting of:

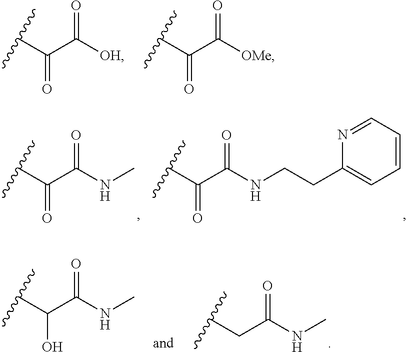

In some embodiments of the invention, G⁹ is selected from the group consisting of:

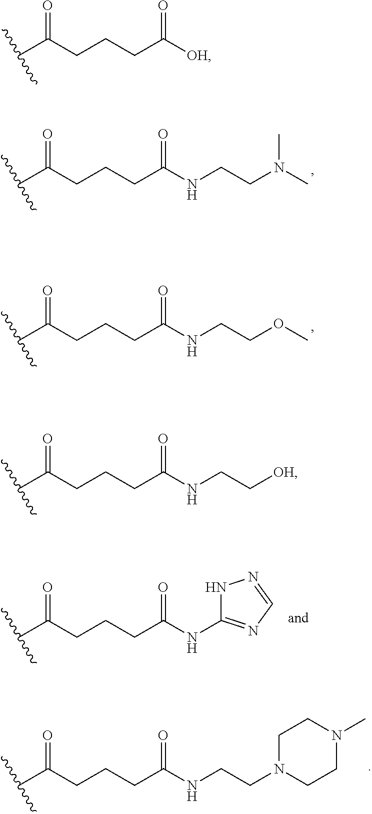 [SMILES: *C(=O)CCCC(=O)O ; *C(=O)CCCC(=O)NCCN(C)C ; *C(=O)CCCC(=O)NCCOC ; *C(=O)CCCC(=O)NCCO ; *C(=O)CCCC(=O)Nc1ncn[nH]1 ; *C(=O)CCCC(=O)NCCN1CCN(C)CC1]

In some embodiments of the present invention, G⁴ of general formula (1) is a bond and G⁵ of general formula (1) is

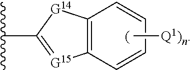

In still other embodiments of the present invention, G⁴ of general formula (1) is a bond, G⁵ of general formula (1) is

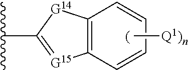

and G³ is CG⁹.

In some embodiments, G³ is N or CH, and G⁵ comprises at least one of Q¹, Q², Q³, Q⁴, Q⁵, Q⁶, Q⁷, and Q⁸, n is at least 2, a first Q¹, Q², Q³, Q⁴, Q⁵, Q⁶, Q⁷, and Q⁸ is selected from the group consisting of —OR^{26′} and —O—(C₁₋₆)alkyl-NR^{27′}R^{28′} wherein each R^{26′} is independently selected from the group consisting of substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C(₂₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and each R^{27′}, and R^{28′} are independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C(₆₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and each pair: R^{27′} and R^{28′}, may alternately be and independently as a pair be a 3-7 membered substituted heterocarbocyclic ring or a 3-7 membered unsubstituted heterocarbocyclic ring, and a second Q¹, Q², Q³, Q⁴, Q⁵, Q⁶, Q⁷, and Q⁸ is selected from the group consisting of F, —OR^{26′} and —O—(C₁₋₆)alkyl-NR^{27′}R^{28′} and each R^{26′}, R^{27′}, and R^{28′} are independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and each pair: R^{27′} and R^{28′} may alternately be and independently as a pair be a 3-7 membered substituted heterocarbocyclic ring or a 3-7 membered unsubstituted heterocarbocyclic ring.

In some embodiments, G³ is N or CH, and G⁵ comprises a Q¹, Q², Q³, Q⁴, Q⁵, Q⁶, Q⁷, Q⁸, Q⁹ or Q¹⁰ that is —O—(C₁₋₆)alkyl-NR^{27′}R^{28′} wherein each R^{27′}, and R^{28′} are independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₆₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and each pair: R^{27′} and R^{28′}, may alternately be and independently as a pair be a 3-7 membered substituted heterocarbocyclic ring or a 3-7 membered unsubstituted heterocarbocyclic ring.

In some embodiments, G³ is N or CH, and G⁴ is other than

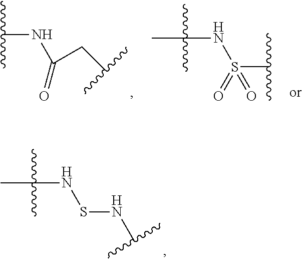 [SMILES: *CC(=O)N* ; *NS(*)(=O)=O ; *NSN*]

and:

(i) G⁵ comprises a Q¹, Q², Q³, Q⁴, Q⁵, Q⁶, Q⁷, Q⁸, Q⁹ r Q¹⁰ that is —O—(C₁₋₆)alkyl-NR^{27′}R^{28′} wherein each R^{27′}, and R^{28′} are independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₆₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and each pair: R^{27′} and R^{28′}, may alternately be and independently as a pair be a 3-7 membered substituted heterocarbocyclic ring or a 3-7 membered unsubstituted heterocarbocyclic ring; or

(ii) G⁵ is

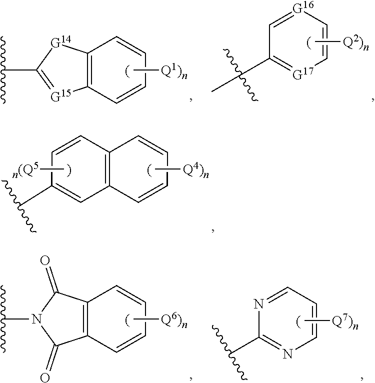

or a 5-membered heteroaryl substituted with (Q⁸)ₙ and containing 1 or 2 heteroatoms each heteroatom independently selected from N, O and S; at least one ‘n’ is at least 2, such that G⁵ comprises at least a first Q¹, Q², Q⁴, Q⁵, Q⁶, Q⁷, or Q⁸, and at least a second Q¹, Q², Q⁴, Q⁵, Q⁶, Q⁷, or Q⁸, which may be the same or different, wherein the first Q¹, Q², Q⁴, Q⁵, Q⁷, or Q⁸ is —OR^{26′} wherein each R^{26′} is independently selected from the group consisting of substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₂₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and the second Q¹, Q², Q⁴, Q⁵, Q⁶, or Q⁸ is selected from the group consisting of F, Br, Cl and —OR^{26′} wherein each R^{26′} is selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; or

(iii) G⁵ is

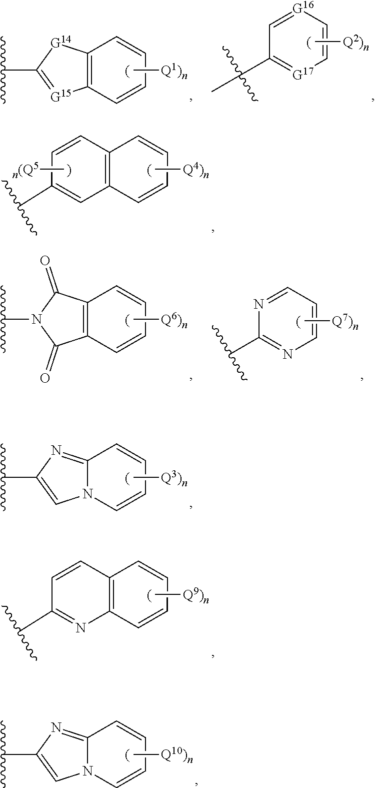

or a 5-membered heteroaryl substituted with (Q⁸)ₙ and containing 1 or 2 heteroatoms each heteroatom independently selected from N, O and S; at least one ‘n’ is 2, such that G⁵ comprises at least a first Q¹, Q², Q⁴, Q⁵, Q⁶, Q⁷, Q⁸, Q⁹ or Q¹⁰, and at least a second Q¹, Q², Q³, Q⁴, Q⁵, Q⁶, Q⁷, Q⁸, Q⁹ or Q¹⁰, which may be the same or different, wherein the first Q¹, Q², Q³, Q⁴, Q⁵, Q⁶, Q⁷, Q⁸, Q⁹ or Q¹⁰ is —OR^{26′} wherein each R^{26′} is independently selected from the group consisting of substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₂₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and the second Q¹, Q², Q³, Q⁴, Q⁵, Q⁶, Q⁷, Q⁸, Q⁹ or Q¹⁰ is selected from the group consisting of F, Br, Cl, —OR^{26′} and each R^{26′} is independently selected from the group consisting of: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl.

Moieties with embodiments of the present invention as set out herein, wherein an alkyl is described, the alkyl may be a homoalkyl or an alkyl. For example, a C₁₋₆ alkyl may be a C₁₋₆ homoalkyl and a C₃₋₁₁ aralkyl may be a C₃₋₁₁ arylhomoalkyl.

Moieties within embodiments of the present invention as set out herein comprise moieties which may be substituted. These substituents may be substituted in accordance with the definition for substituents and substitutions as set out herein.

Some of the molecules of general formula (1) have one or more chiral centres. The present invention contemplates and includes without limitation, optically pure compounds as well as racemic mixtures and mixtures of varying proportions of the R and S configurations of each chiral centre.

Some of the molecules of the general formula (1) may have one or more counterions. Such counterions are readily understood by a person of skill in the art and the replacement and/or exchange and/or presence of such a counterion may be adapted by a person of skill in the art in accordance with such understanding.

Some non-limiting examples of embodiments of formula (1) are provided in Tables 1 and 2 below.

TABLE 1

| Compound | |
|---|---|
| # | Chemical Structure |
| 1 | 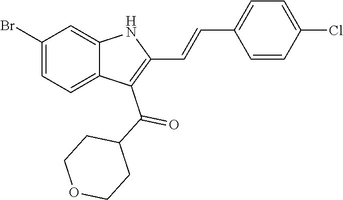 [SMILES: O=C(c1c(/C=C/c2ccc(Cl)cc2)[nH]c2cc(Br)ccc12)C1CCOCC1] |
| 2 | 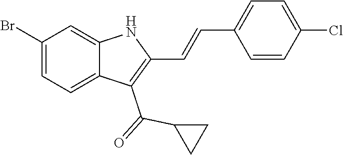 [SMILES: O=C(c1c(/C=C/c2ccc(Cl)cc2)[nH]c2cc(Br)ccc12)C1CC1] |
| 3 | 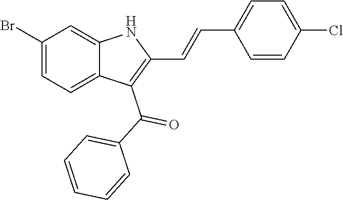 [SMILES: O=C(c1ccccc1)c1c(/C=C/c2ccc(Cl)cc2)[nH]c2cc(Br)ccc12] |
| 4 | 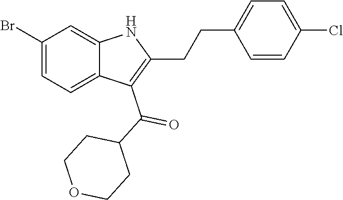 [SMILES: O=C(c1c(CCc2ccc(Cl)cc2)[nH]c2cc(Br)ccc12)C1CCOCC1] |
| 5 | 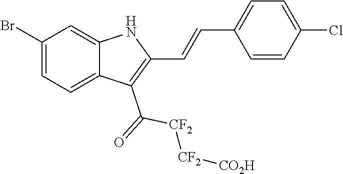 |
| 6 | 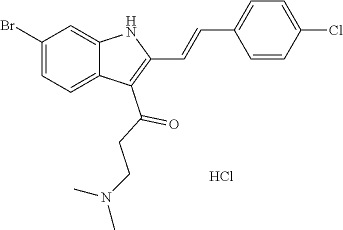 [SMILES: CN(C)CCC(=O)c1c(/C=C/c2ccc(Cl)cc2)[nH]c2cc(Br)ccc12 ; Cl] |
| 7 | 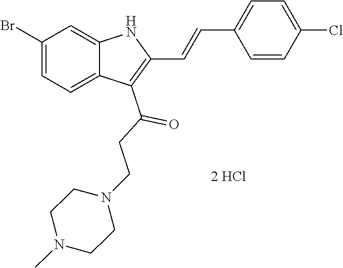 [SMILES: CN1CCN(CCC(=O)c2c(/C=C/c3ccc(Cl)cc3)[nH]c3cc(Br)ccc23)CC1 ; "2 HCl"] |
| 8 | 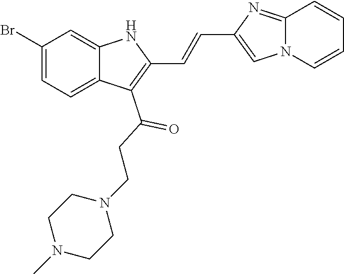 [SMILES: CN1CCN(CCC(=O)c2c(/C=C/c3cn4ccccc4n3)[nH]c3cc(Br)ccc23)CC1] |
| 9 | 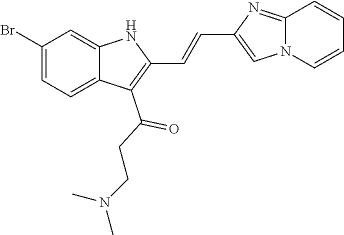 [SMILES: CN(C)CCC(=O)c1c(/C=C/c2cn3ccccc3n2)[nH]c2cc(Br)ccc12] |
| 10 | 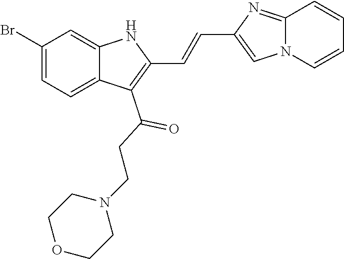 [SMILES: O=C(CCN1CCOCC1)c1c(/C=C/c2cn3ccccc3n2)[nH]c2cc(Br)ccc12] |
| 11 | 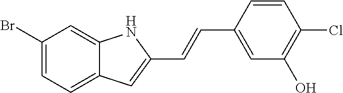 [SMILES: Oc1cc(/C=C/c2cc3ccc(Br)cc3[nH]2)ccc1Cl] |
| 12 | 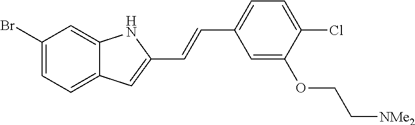 [SMILES: CN(C)CCOc1cc(/C=C/c2cc3ccc(Br)cc3[nH]2)ccc1Cl] |
| 13 | 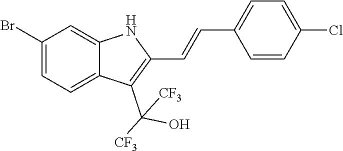 [SMILES: OC(c1c(/C=C/c2ccc(Cl)cc2)[nH]c2cc(Br)ccc12)(C(F)(F)F)C(F)(F)F] |
| 14 | 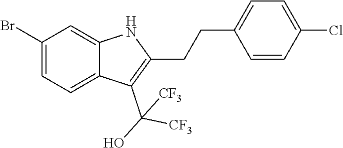 [SMILES: OC(c1c(CCc2ccc(Cl)cc2)[nH]c2cc(Br)ccc12)(C(F)(F)F)C(F)(F)F] |
| 15 | 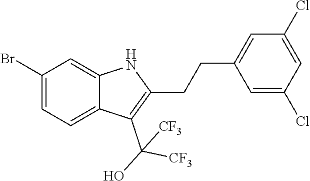 [SMILES: OC(c1c(CCc2cc(Cl)cc(Cl)c2)[nH]c2cc(Br)ccc12)(C(F)(F)F)C(F)(F)F] |
| 16 | 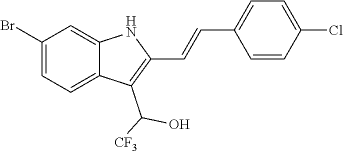 [SMILES: OC(c1c(/C=C/c2ccc(Cl)cc2)[nH]c2cc(Br)ccc12)C(F)(F)F] |
| 17 | 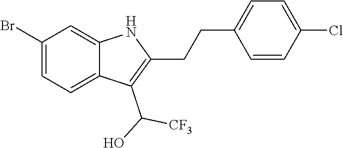 [SMILES: OC(c1c(CCc2ccc(Cl)cc2)[nH]c2cc(Br)ccc12)C(F)(F)F] |
| 18 | 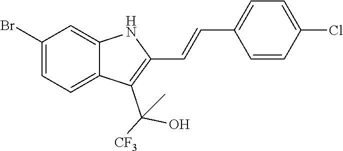 [SMILES: CC(O)(c1c(/C=C/c2ccc(Cl)cc2)[nH]c2cc(Br)ccc12)C(F)(F)F] |
| 19 | 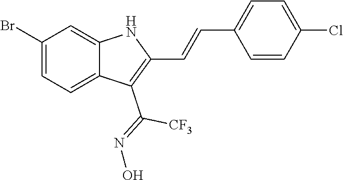 [SMILES: O/N=C(/c1c(/C=C/c2ccc(Cl)cc2)[nH]c2cc(Br)ccc12)C(F)(F)F] |
| 20 | 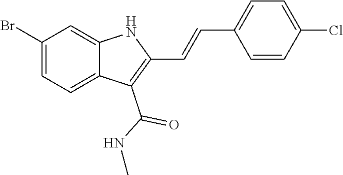 [SMILES: CNC(=O)c1c(/C=C/c2ccc(Cl)cc2)[nH]c2cc(Br)ccc12] |
| 21 | 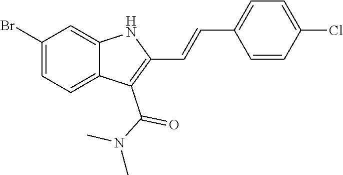 [SMILES: CN(C)C(=O)c1c(/C=C/c2ccc(Cl)cc2)[nH]c2cc(Br)ccc12] |
| 22 | 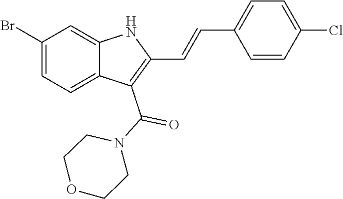 [SMILES: O=C(c1c(/C=C/c2ccc(Cl)cc2)[nH]c2cc(Br)ccc12)N1CCOCC1] |
| 23 | 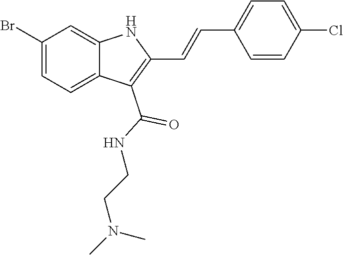 [SMILES: CN(C)CCNC(=O)c1c(/C=C/c2ccc(Cl)cc2)[nH]c2cc(Br)ccc12] |
| 24 | 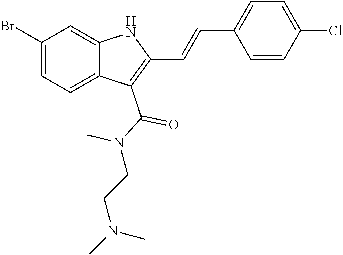 [SMILES: CN(C)CCN(C)C(=O)c1c(/C=C/c2ccc(Cl)cc2)[nH]c2cc(Br)ccc12] |
| 25 | 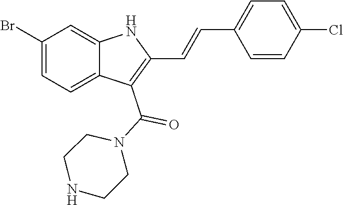 [SMILES: O=C(c1c(/C=C/c2ccc(Cl)cc2)[nH]c2cc(Br)ccc12)N1CCNCC1] |
| 26 | 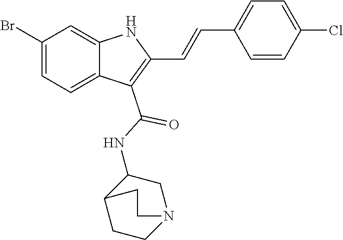 [SMILES: O=C(NC1CN2CCC1CC2)c1c(/C=C/c2ccc(Cl)cc2)[nH]c2cc(Br)ccc12] |
| 27 | 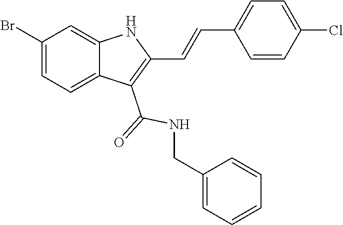 [SMILES: O=C(NCc1ccccc1)c1c(/C=C/c2ccc(Cl)cc2)[nH]c2cc(Br)ccc12] |
| 28 | 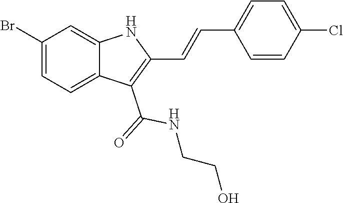 [SMILES: O=C(NCCO)c1c(/C=C/c2ccc(Cl)cc2)[nH]c2cc(Br)ccc12] |
| 29 | 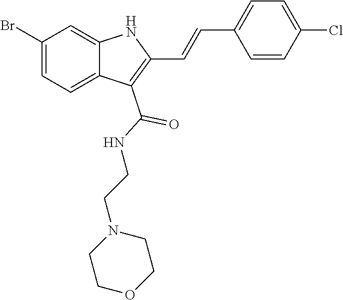 [SMILES: O=C(NCCN1CCOCC1)c1c(/C=C/c2ccc(Cl)cc2)[nH]c2cc(Br)ccc12] |
| 30 | 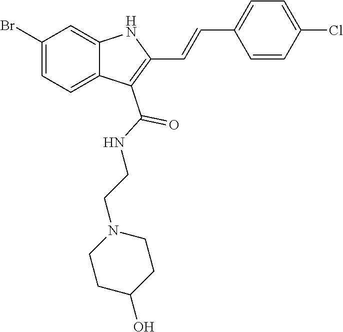 [SMILES: O=C(NCCN1CCC(O)CC1)c1c(/C=C/c2ccc(Cl)cc2)[nH]c2cc(Br)ccc12] |
| 31 | 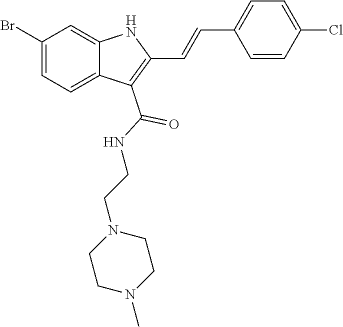 [SMILES: CN1CCN(CCNC(=O)c2c(/C=C/c3ccc(Cl)cc3)[nH]c3cc(Br)ccc23)CC1] |
| 32 | 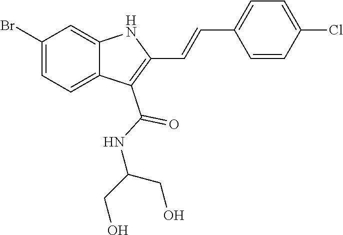 [SMILES: O=C(NC(CO)CO)c1c(/C=C/c2ccc(Cl)cc2)[nH]c2cc(Br)ccc12] |
| 33 | 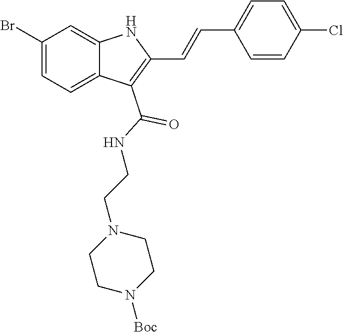 [SMILES: CC(C)(C)OC(=O)N1CCN(CCNC(=O)c2c(/C=C/c3ccc(Cl)cc3)[nH]c3cc(Br)ccc23)CC1] |
| 34 | 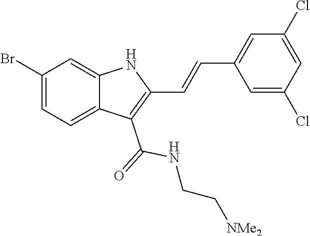 [SMILES: CN(C)CCNC(=O)c1c(/C=C/c2cc(Cl)cc(Cl)c2)[nH]c2cc(Br)ccc12] |
| 35 | 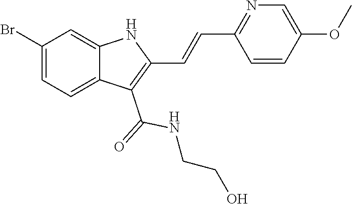 [SMILES: COc1ccc(/C=C/c2[nH]c3cc(Br)ccc3c2C(=O)NCCO)nc1] |
| 36 | 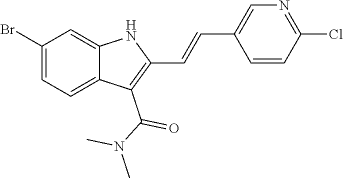 [SMILES: CN(C)C(=O)c1c(/C=C/c2ccc(Cl)nc2)[nH]c2cc(Br)ccc12] |
| 37 | 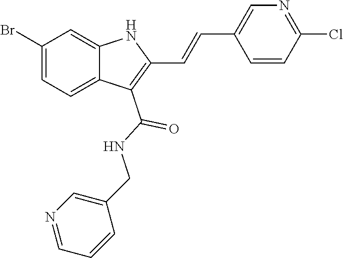 [SMILES: O=C(NCc1cccnc1)c1c(/C=C/c2ccc(Cl)nc2)[nH]c2cc(Br)ccc12] |
| 38 | 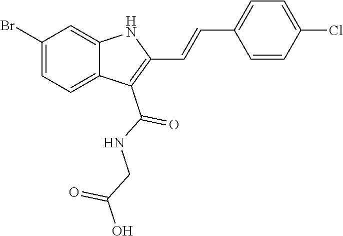 [SMILES: O=C(O)CNC(=O)c1c(/C=C/c2ccc(Cl)cc2)[nH]c2cc(Br)ccc12] |
| 39 | 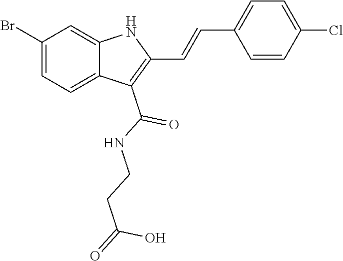 [SMILES: O=C(O)CCNC(=O)c1c(/C=C/c2ccc(Cl)cc2)[nH]c2cc(Br)ccc12] |
| 40 | 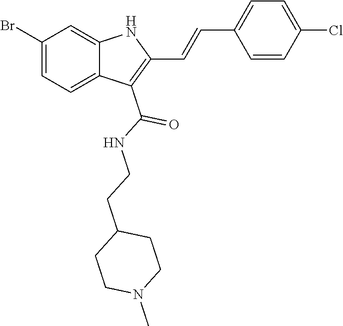 [SMILES: CN1CCC(CCNC(=O)c2c(/C=C/c3ccc(Cl)cc3)[nH]c3cc(Br)ccc23)CC1] |
| 41 | 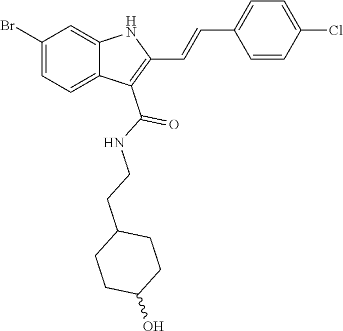 [SMILES: O=C(NCCC1CCC(O)CC1)c1c(/C=C/c2ccc(Cl)cc2)[nH]c2cc(Br)ccc12] |
| 42 | 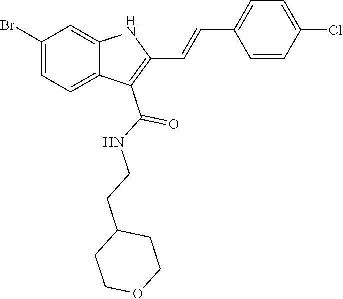 [SMILES: O=C(NCCC1CCOCC1)c1c(/C=C/c2ccc(Cl)cc2)[nH]c2cc(Br)ccc12] |
| 43 | 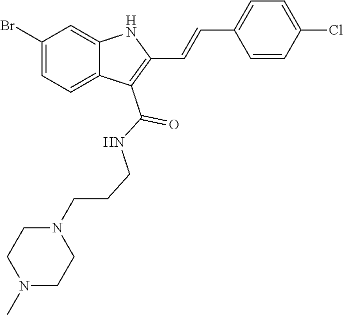 [SMILES: CN1CCN(CCCNC(=O)c2c(/C=C/c3ccc(Cl)cc3)[nH]c3cc(Br)ccc23)CC1] |
| 44 | 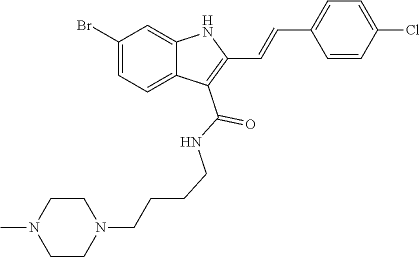 [SMILES: CN1CCN(CCCCNC(=O)c2c(/C=C/c3ccc(Cl)cc3)[nH]c3cc(Br)ccc23)CC1] |
| 45 | 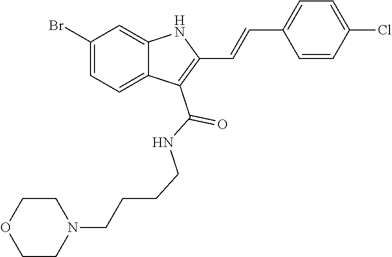 [SMILES: O=C(NCCCCN1CCOCC1)c1c(/C=C/c2ccc(Cl)cc2)[nH]c2cc(Br)ccc12] |
| 46 | 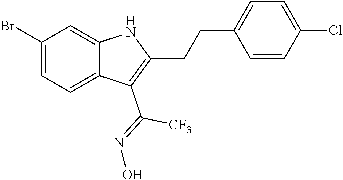 [SMILES: O/N=C(/c1c(CCc2ccc(Cl)cc2)[nH]c2cc(Br)ccc12)C(F)(F)F] |
| 47 | 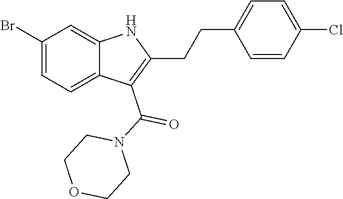 [SMILES: O=C(c1c(CCc2ccc(Cl)cc2)[nH]c2cc(Br)ccc12)N1CCOCC1] |
| 48 | 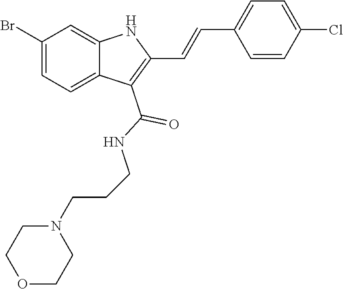 [SMILES: O=C(NCCCN1CCOCC1)c1c(/C=C/c2ccc(Cl)cc2)[nH]c2cc(Br)ccc12] |
| 49 | 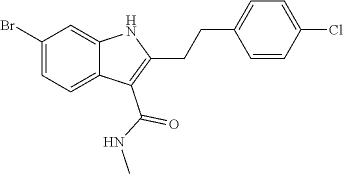 [SMILES: CNC(=O)c1c(CCc2ccc(Cl)cc2)[nH]c2cc(Br)ccc12] |
| 50 | 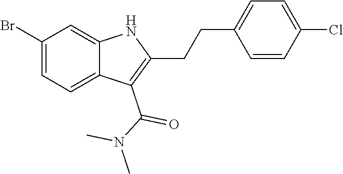 [SMILES: CN(C)C(=O)c1c(CCc2ccc(Cl)cc2)[nH]c2cc(Br)ccc12] |
| 51 | 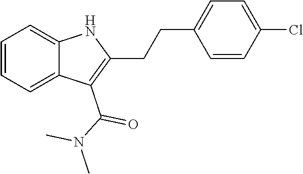 [SMILES: CN(C)C(=O)c1c(CCc2ccc(Cl)cc2)[nH]c2ccccc12] |
| 52 | 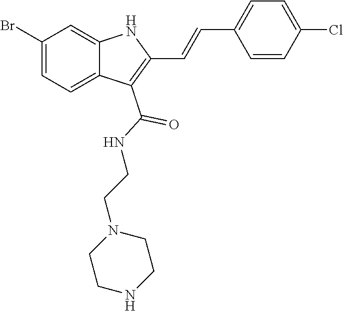 [SMILES: O=C(NCCN1CCNCC1)c1c(/C=C/c2ccc(Cl)cc2)[nH]c2cc(Br)ccc12] |
| 53 | 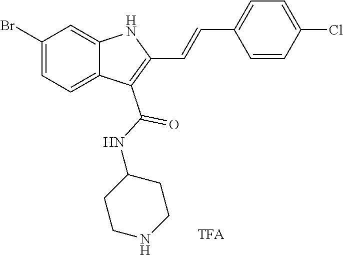 [SMILES: O=C(NC1CCNCC1)c1c(/C=C/c2ccc(Cl)cc2)[nH]c2cc(Br)ccc12] |
| 54 | 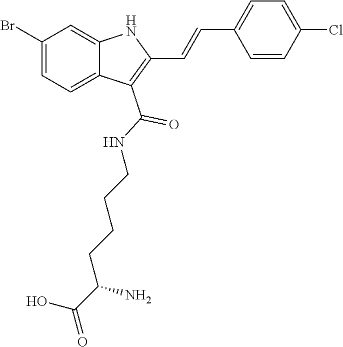 [SMILES: N[C@@H](CCCCNC(=O)c1c(/C=C/c2ccc(Cl)cc2)[nH]c2cc(Br)ccc12)C(=O)O] |
| 55 | 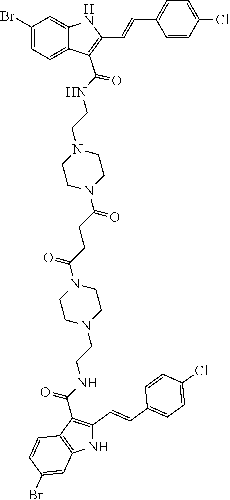 [SMILES: O=C(NCCN1CCN(C(=O)CCC(=O)N2CCN(CCNC(=O)c3c(/C=C/c4ccc(Cl)cc4)[nH]c4cc(Br)ccc34)CC2)CC1)c1c(/C=C/c2ccc(Cl)cc2)[nH]c2cc(Br)ccc12] |
| 56 | 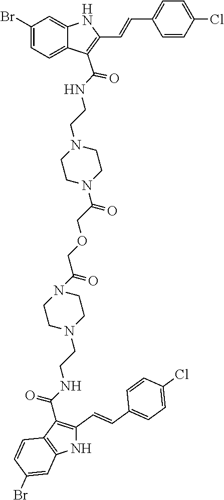 [SMILES: O=C(NCCN1CCN(C(=O)COCC(=O)N2CCN(CCNC(=O)c3c(/C=C/c4ccc(Cl)cc4)[nH]c4cc(Br)ccc34)CC2)CC1)c1c(/C=C/c2ccc(Cl)cc2)[nH]c2cc(Br)ccc12] |
| 57 | 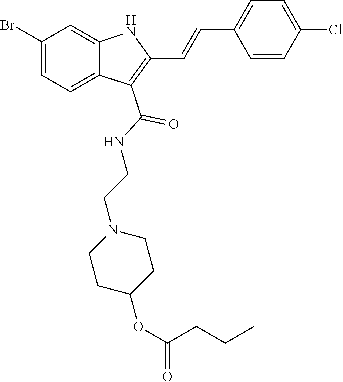 [SMILES: CCCC(=O)OC1CCN(CCNC(=O)c2c(/C=C/c3ccc(Cl)cc3)[nH]c3cc(Br)ccc23)CC1] |
| 58 | 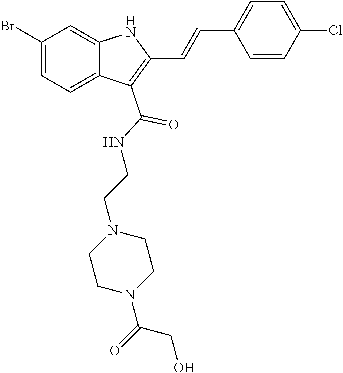 [SMILES: O=C(NCCN1CCN(C(=O)CO)CC1)c1c(/C=C/c2ccc(Cl)cc2)[nH]c2cc(Br)ccc12] |
| 116 | 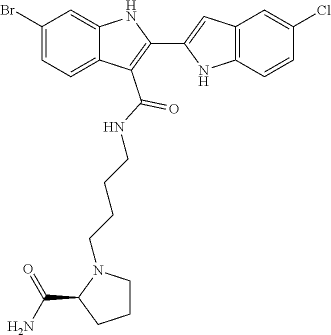 [SMILES: NC(=O)[C@@H]1CCCN1CCCCNC(=O)c1c(-c2cc3cc(Cl)ccc3[nH]2)[nH]c2cc(Br)ccc12] |
| 117 | 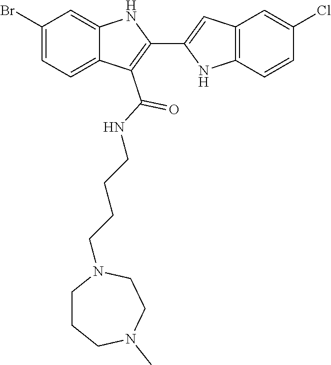 [SMILES: CN1CCCN(CCCCNC(=O)c2c(-c3cc4cc(Cl)ccc4[nH]3)[nH]c3cc(Br)ccc23)CC1] |
| 118 | 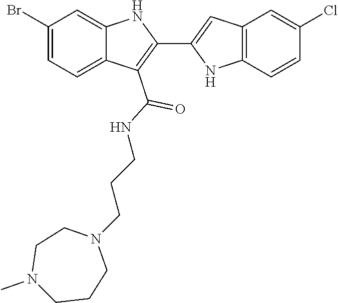 [SMILES: CN1CCCN(CCCNC(=O)c2c(-c3cc4cc(Cl)ccc4[nH]3)[nH]c3cc(Br)ccc23)CC1] |
| 119 | 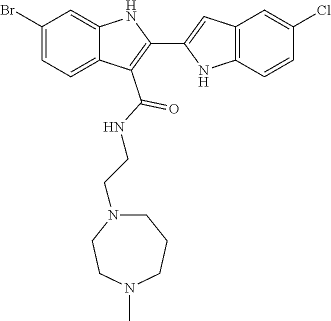 [SMILES: CN1CCCN(CCNC(=O)c2c(-c3cc4cc(Cl)ccc4[nH]3)[nH]c3cc(Br)ccc23)CC1] |
| 120 | 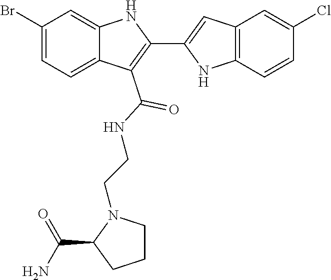 [SMILES: NC(=O)[C@@H]1CCCN1CCNC(=O)c1c(-c2cc3cc(Cl)ccc3[nH]2)[nH]c2cc(Br)ccc12] |
| 121 | 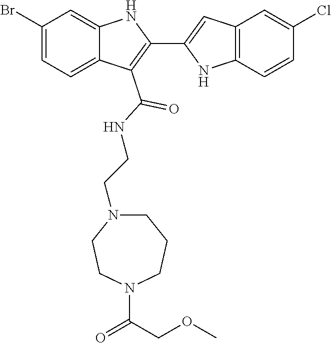 [SMILES: COCC(=O)N1CCCN(CCNC(=O)c2c(-c3cc4cc(Cl)ccc4[nH]3)[nH]c3cc(Br)ccc23)CC1] |
| 122 | 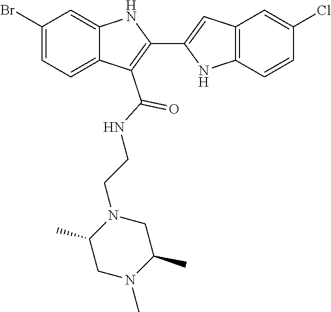 [SMILES: C[C@@H]1CN(CCNC(=O)c2c(-c3cc4cc(Cl)ccc4[nH]3)[nH]c3cc(Br)ccc23)[C@@H](C)CN1C] |
| 123 | 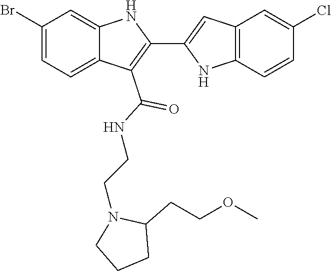 [SMILES: COCCC1CCCN1CCNC(=O)c1c(-c2cc3cc(Cl)ccc3[nH]2)[nH]c2cc(Br)ccc12] |
| 124 | 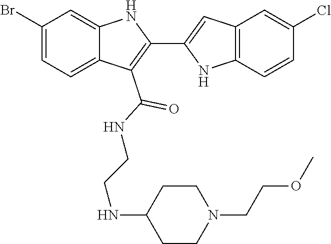 [SMILES: COCCN1CCC(NCCNC(=O)c2c(-c3cc4cc(Cl)ccc4[nH]3)[nH]c3cc(Br)ccc23)CC1] |
| 125 | 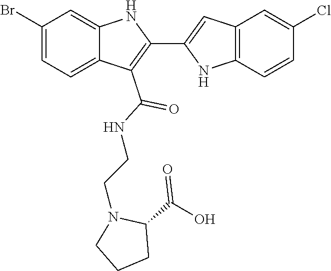 [SMILES: O=C(NCCN1CCC[C@H]1C(=O)O)c1c(-c2cc3cc(Cl)ccc3[nH]2)[nH]c2cc(Br)ccc12] |
| 126 | 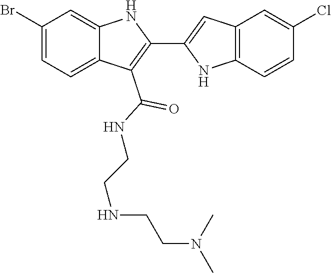 [SMILES: CN(C)CCNCCNC(=O)c1c(-c2cc3cc(Cl)ccc3[nH]2)[nH]c2cc(Br)ccc12] |
| 127 | 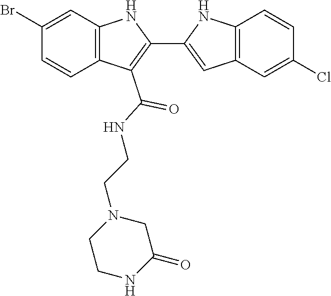 [SMILES: O=C1CN(CCNC(=O)c2c(-c3cc4cc(Cl)ccc4[nH]3)[nH]c3cc(Br)ccc23)CCN1] |
| 128 | 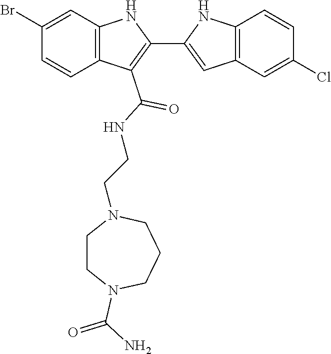 [SMILES: NC(=O)N1CCCN(CCNC(=O)c2c(-c3cc4cc(Cl)ccc4[nH]3)[nH]c3cc(Br)ccc23)CC1] |
| 129 | 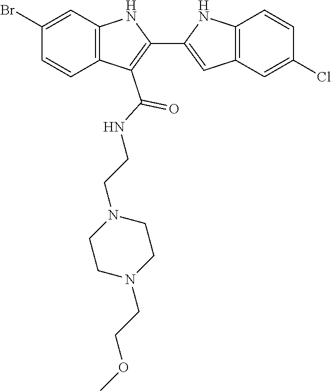 [SMILES: COCCN1CCN(CCNC(=O)c2c(-c3cc4cc(Cl)ccc4[nH]3)[nH]c3cc(Br)ccc23)CC1] |
| 145 | 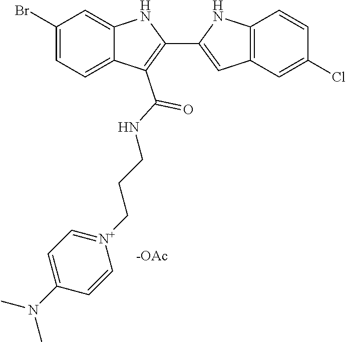 [SMILES: CN(C)c1cc[n+](CCCNC(=O)c2c(-c3cc4cc(Cl)ccc4[nH]3)[nH]c3cc(Br)ccc23)cc1] |
| 147 | 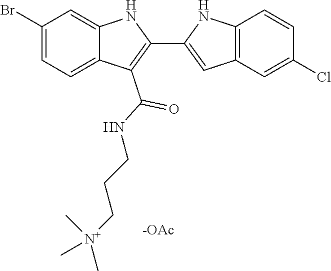 [SMILES: C[N+](C)(C)CCCNC(=O)c1c(-c2cc3cc(Cl)ccc3[nH]2)[nH]c2cc(Br)ccc12] |
| 149 | 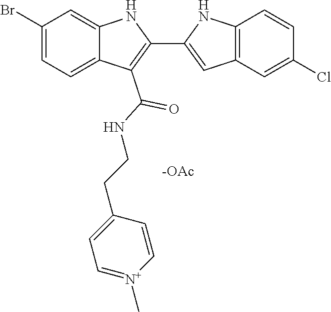 [SMILES: C[n+]1ccc(CCNC(=O)c2c(-c3cc4cc(Cl)ccc4[nH]3)[nH]c3cc(Br)ccc23)cc1] |
| 151 | 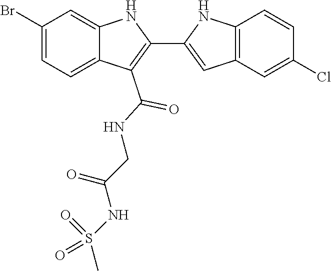 [SMILES: CS(=O)(=O)NC(=O)CNC(=O)c1c(-c2cc3cc(Cl)ccc3[nH]2)[nH]c2cc(Br)ccc12] |
| 153 | 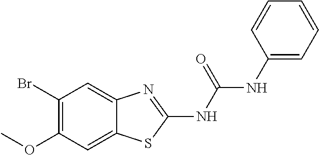 [SMILES: COc1cc2sc(NC(=O)Nc3ccccc3)nc2cc1Br] |
| 155 | 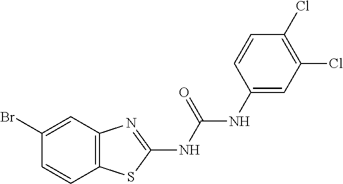 [SMILES: O=C(Nc1ccc(Cl)c(Cl)c1)Nc1nc2cc(Br)ccc2s1] |
| 157 | 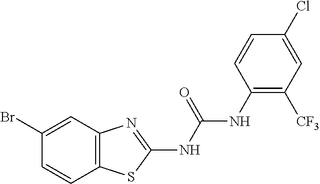 [SMILES: O=C(Nc1nc2cc(Br)ccc2s1)Nc1ccc(Cl)cc1C(F)(F)F] |
| 159 | 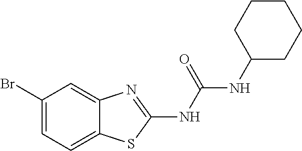 [SMILES: O=C(Nc1nc2cc(Br)ccc2s1)NC1CCCCC1] |
| 161 | 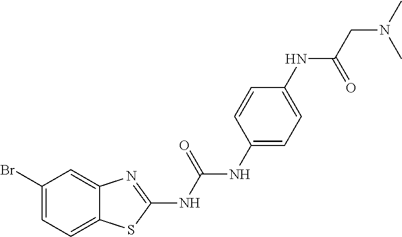 [SMILES: CN(C)CC(=O)Nc1ccc(NC(=O)Nc2nc3cc(Br)ccc3s2)cc1] |
| 163 | 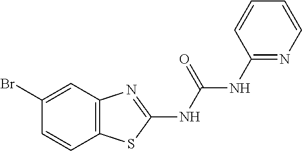 [SMILES: O=C(Nc1ccccn1)Nc1nc2cc(Br)ccc2s1] |
| 165 | 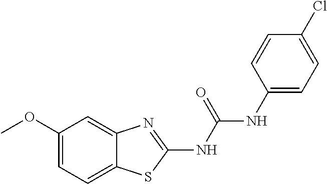 [SMILES: COc1ccc2sc(NC(=O)Nc3ccc(Cl)cc3)nc2c1] |
| 167 | 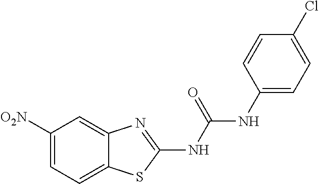 [SMILES: O=C(Nc1ccc(Cl)cc1)Nc1nc2cc([N+](=O)[O-])ccc2s1] |
| 169 | 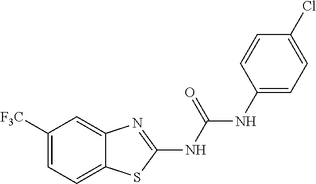 [SMILES: O=C(Nc1ccc(Cl)cc1)Nc1nc2cc(C(F)(F)F)ccc2s1] |
| 171 | 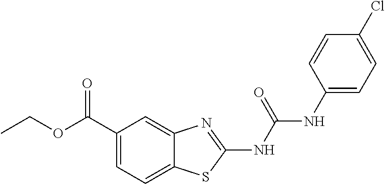 [SMILES: CCOC(=O)c1ccc2sc(NC(=O)Nc3ccc(Cl)cc3)nc2c1] |
| 173 | 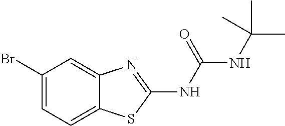 [SMILES: CC(C)(C)NC(=O)Nc1nc2cc(Br)ccc2s1] |
| 177 | 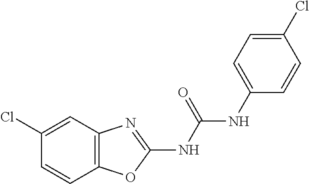 [SMILES: O=C(Nc1ccc(Cl)cc1)Nc1nc2cc(Cl)ccc2o1] |
| 179 | 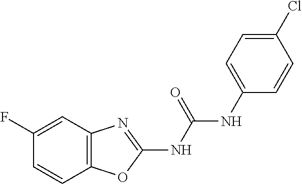 [SMILES: O=C(Nc1ccc(Cl)cc1)Nc1nc2cc(F)ccc2o1] |
| 181 | 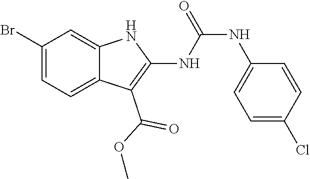 [SMILES: COC(=O)c1c(NC(=O)Nc2ccc(Cl)cc2)[nH]c2cc(Br)ccc12] |
| 183 | 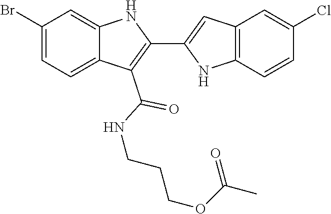 [SMILES: CC(=O)OCCCNC(=O)c1c(-c2cc3cc(Cl)ccc3[nH]2)[nH]c2cc(Br)ccc12] |
| 185 | 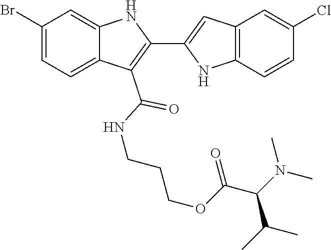 [SMILES: CC(C)[C@@H](C(=O)OCCCNC(=O)c1c(-c2cc3cc(Cl)ccc3[nH]2)[nH]c2cc(Br)ccc12)N(C)C] |
| 187 | 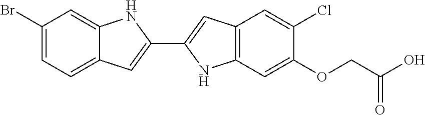 [SMILES: O=C(O)COc1cc2[nH]c(-c3cc4ccc(Br)cc4[nH]3)cc2cc1Cl] |
| 189 | 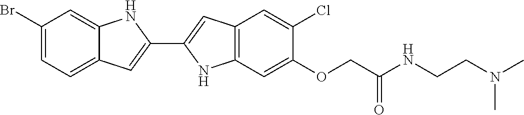 [SMILES: CN(C)CCNC(=O)COc1cc2[nH]c(-c3cc4ccc(Br)cc4[nH]3)cc2cc1Cl] |
| 191 | 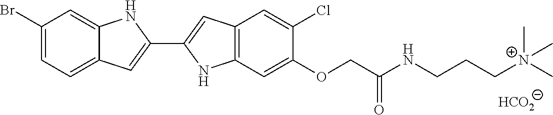 |
| 193 | 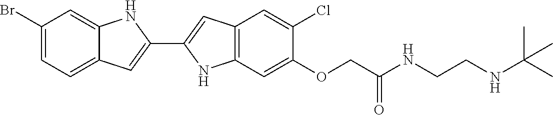 [SMILES: CC(C)(C)NCCNC(=O)COc1cc2[nH]c(-c3cc4ccc(Br)cc4[nH]3)cc2cc1Cl] |
| 195 | 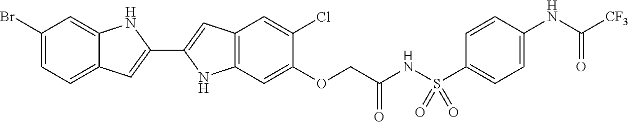 [SMILES: O=C(COc1cc2[nH]c(-c3cc4ccc(Br)cc4[nH]3)cc2cc1Cl)NS(=O)(=O)c1ccc(NC(=O)C(F)(F)F)cc1] |
| 197 | 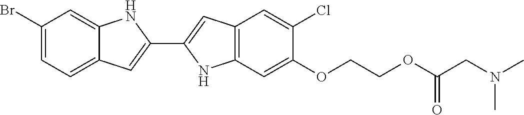 [SMILES: CN(C)CC(=O)OCCOc1cc2[nH]c(-c3cc4ccc(Br)cc4[nH]3)cc2cc1Cl] |
| 199 | 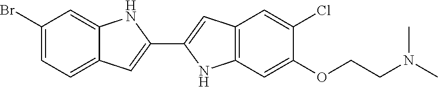 [SMILES: CN(C)CCOc1cc2[nH]c(-c3cc4ccc(Br)cc4[nH]3)cc2cc1Cl] |
| 201 | 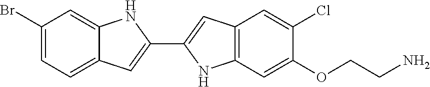 [SMILES: NCCOc1cc2[nH]c(-c3cc4ccc(Br)cc4[nH]3)cc2cc1Cl] |
| 203 | 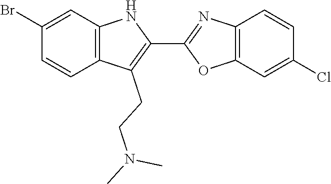 [SMILES: CN(C)CCc1c(-c2nc3ccc(Cl)cc3o2)[nH]c2cc(Br)ccc12] |
| 205 | 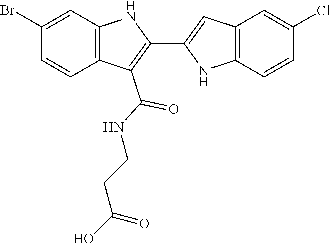 [SMILES: O=C(O)CCNC(=O)c1c(-c2cc3cc(Cl)ccc3[nH]2)[nH]c2cc(Br)ccc12] |
| 207 | 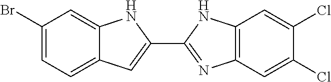 [SMILES: Clc1cc2nc(-c3cc4ccc(Br)cc4[nH]3)[nH]c2cc1Cl] |
| 209 | 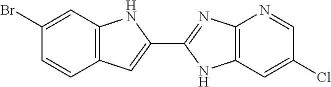 [SMILES: Clc1cnc2nc(-c3cc4ccc(Br)cc4[nH]3)[nH]c2c1] |
| 211 | 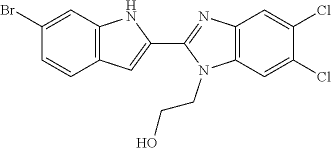 [SMILES: OCCn1c(-c2cc3ccc(Br)cc3[nH]2)nc2cc(Cl)c(Cl)cc21] |
| 213 | 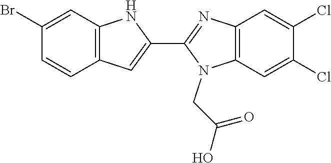 [SMILES: O=C(O)Cn1c(-c2cc3ccc(Br)cc3[nH]2)nc2cc(Cl)c(Cl)cc21] |
| 215 | 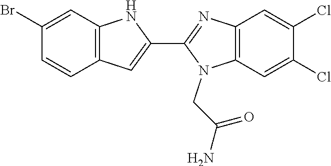 [SMILES: NC(=O)Cn1c(-c2cc3ccc(Br)cc3[nH]2)nc2cc(Cl)c(Cl)cc21] |
| 217 | 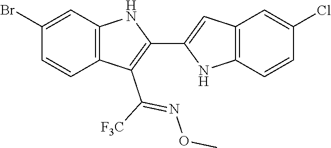 [SMILES: CO/N=C(/c1c(-c2cc3cc(Cl)ccc3[nH]2)[nH]c2cc(Br)ccc12)C(F)(F)F] |
| 219 | 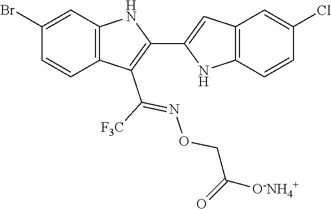 |
| 221 | 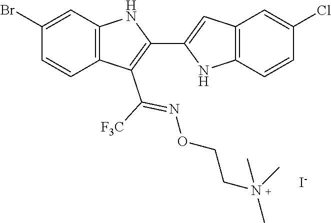 |
| 223 | 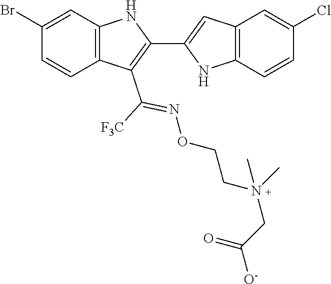 |
| 225 | 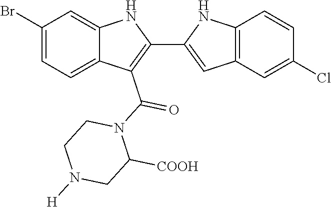 [SMILES: O=C(O)C1CNCCN1C(=O)c1c(-c2cc3cc(Cl)ccc3[nH]2)[nH]c2cc(Br)ccc12] |
| 227 | 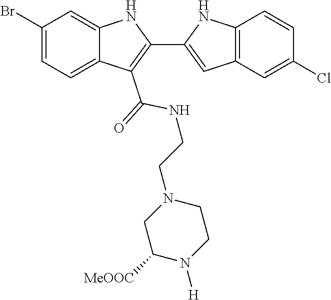 [SMILES: COC(=O)[C@@H]1CN(CCNC(=O)c2c(-c3cc4cc(Cl)ccc4[nH]3)[nH]c3cc(Br)ccc23)CCN1] |
| 229 | 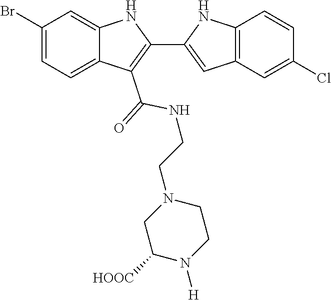 [SMILES: O=C(NCCN1CCN[C@H](C(=O)O)C1)c1c(-c2cc3cc(Cl)ccc3[nH]2)[nH]c2cc(Br)ccc12] |
| 59 | 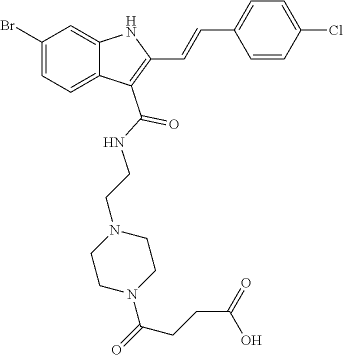 [SMILES: O=C(O)CCC(=O)N1CCN(CCNC(=O)c2c(/C=C/c3ccc(Cl)cc3)[nH]c3cc(Br)ccc23)CC1] |
| 60 | 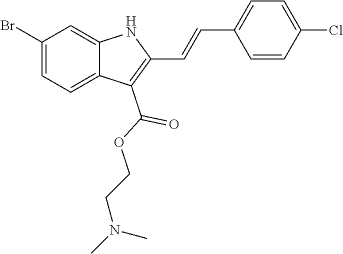 [SMILES: CN(C)CCOC(=O)c1c(/C=C/c2ccc(Cl)cc2)[nH]c2cc(Br)ccc12] |
| 61 | 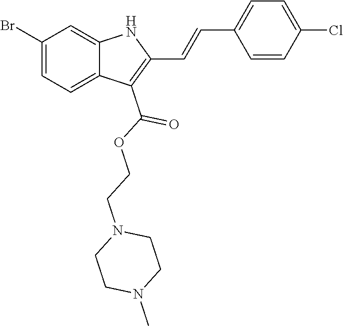 [SMILES: CN1CCN(CCOC(=O)c2c(/C=C/c3ccc(Cl)cc3)[nH]c3cc(Br)ccc23)CC1] |
| 62 | 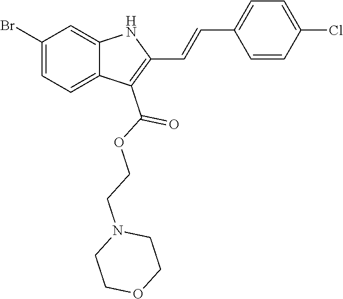 [SMILES: O=C(OCCN1CCOCC1)c1c(/C=C/c2ccc(Cl)cc2)[nH]c2cc(Br)ccc12] |
| 63 | 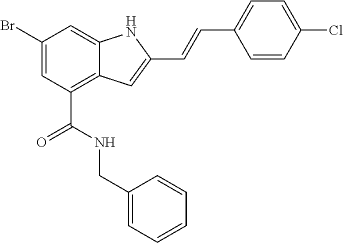 [SMILES: O=C(NCc1ccccc1)c1cc(Br)cc2[nH]c(/C=C/c3ccc(Cl)cc3)cc12] |
| 64 | 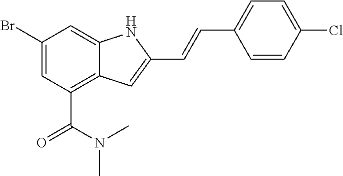 [SMILES: CN(C)C(=O)c1cc(Br)cc2[nH]c(/C=C/c3ccc(Cl)cc3)cc12] |
| 65 | 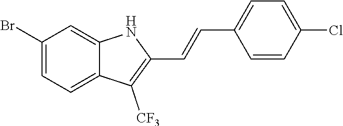 [SMILES: FC(F)(F)c1c(/C=C/c2ccc(Cl)cc2)[nH]c2cc(Br)ccc12] |
| 66 | 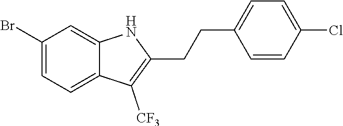 [SMILES: FC(F)(F)c1c(CCc2ccc(Cl)cc2)[nH]c2cc(Br)ccc12] |
| 67 | 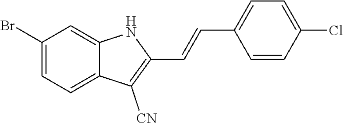 [SMILES: N#Cc1c(/C=C/c2ccc(Cl)cc2)[nH]c2cc(Br)ccc12] |
| 68 | 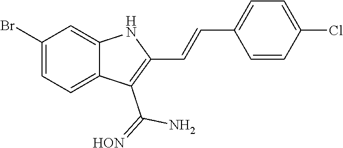 |
| 69 | 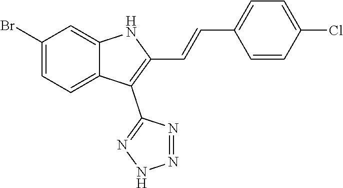 [SMILES: Clc1ccc(/C=C/c2[nH]c3cc(Br)ccc3c2-c2nn[nH]n2)cc1] |
| 70 | 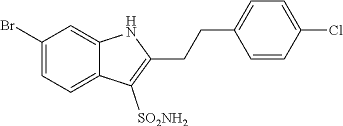 [SMILES: NS(=O)(=O)c1c(CCc2ccc(Cl)cc2)[nH]c2cc(Br)ccc12] |
| 71 | 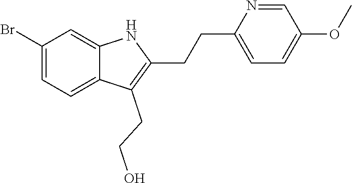 [SMILES: COc1ccc(CCc2[nH]c3cc(Br)ccc3c2CCO)nc1] |
| 72 | 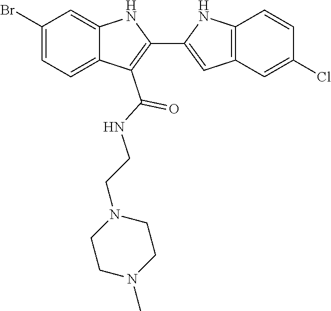 [SMILES: CN1CCN(CCNC(=O)c2c(-c3cc4cc(Cl)ccc4[nH]3)[nH]c3cc(Br)ccc23)CC1] |
| 73 | 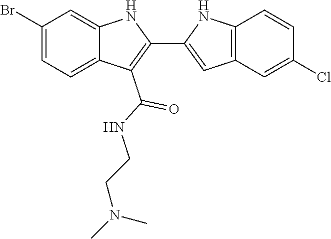 [SMILES: CN(C)CCNC(=O)c1c(-c2cc3cc(Cl)ccc3[nH]2)[nH]c2cc(Br)ccc12] |
| 74 | 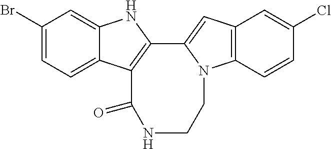 [SMILES: O=C1NCCn2c(cc3cc(Cl)ccc32)-c2[nH]c3cc(Br)ccc3c21] |
| 75 | 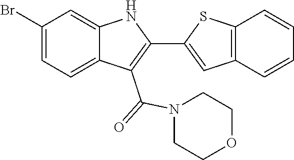 [SMILES: O=C(c1c(-c2cc3ccccc3s2)[nH]c2cc(Br)ccc12)N1CCOCC1] |
| 76 | 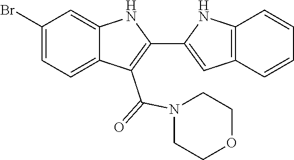 [SMILES: O=C(c1c(-c2cc3ccccc3[nH]2)[nH]c2cc(Br)ccc12)N1CCOCC1] |
| 77 | 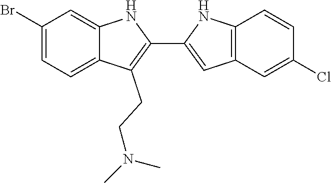 [SMILES: CN(C)CCc1c(-c2cc3cc(Cl)ccc3[nH]2)[nH]c2cc(Br)ccc12] |
| 78 | 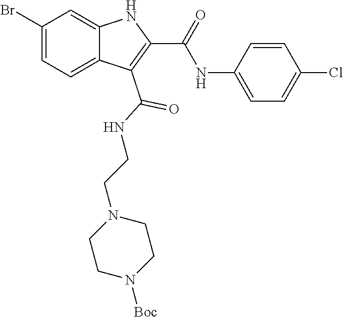 [SMILES: CC(C)(C)OC(=O)N1CCN(CCNC(=O)c2c(C(=O)Nc3ccc(Cl)cc3)[nH]c3cc(Br)ccc23)CC1] |
| 79 | 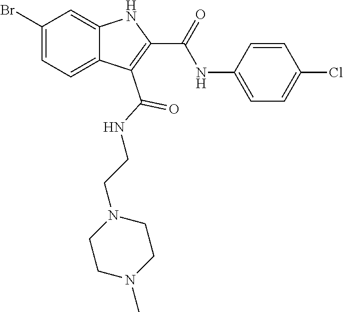 [SMILES: CN1CCN(CCNC(=O)c2c(C(=O)Nc3ccc(Cl)cc3)[nH]c3cc(Br)ccc23)CC1] |
| 80 | 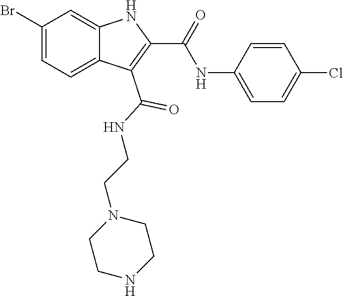 [SMILES: O=C(Nc1ccc(Cl)cc1)c1[nH]c2cc(Br)ccc2c1C(=O)NCCN1CCNCC1] |
| 81 | 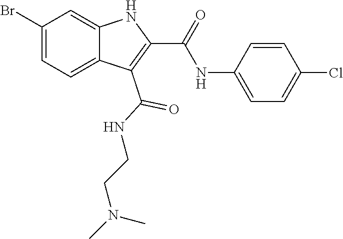 [SMILES: CN(C)CCNC(=O)c1c(C(=O)Nc2ccc(Cl)cc2)[nH]c2cc(Br)ccc12] |
| 82 | 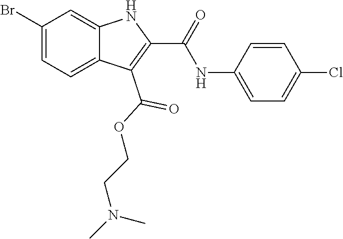 [SMILES: CN(C)CCOC(=O)c1c(C(=O)Nc2ccc(Cl)cc2)[nH]c2cc(Br)ccc12] |
| 83 | 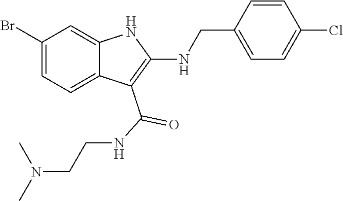 [SMILES: CN(C)CCNC(=O)c1c(NCc2ccc(Cl)cc2)[nH]c2cc(Br)ccc12] |
| 84 | 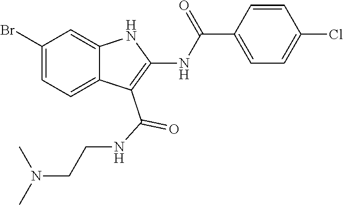 [SMILES: CN(C)CCNC(=O)c1c(NC(=O)c2ccc(Cl)cc2)[nH]c2cc(Br)ccc12] |
| 85 | 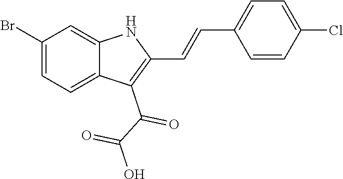 [SMILES: O=C(O)C(=O)c1c(/C=C/c2ccc(Cl)cc2)[nH]c2cc(Br)ccc12] |
| 86 | 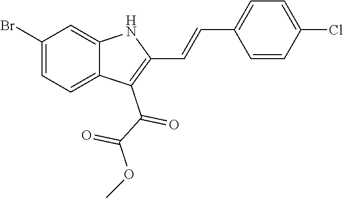 [SMILES: COC(=O)C(=O)c1c(/C=C/c2ccc(Cl)cc2)[nH]c2cc(Br)ccc12] |
| 87 | 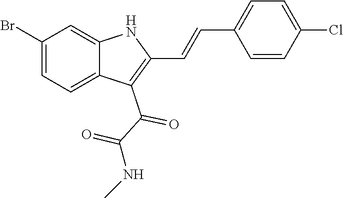 [SMILES: CNC(=O)C(=O)c1c(/C=C/c2ccc(Cl)cc2)[nH]c2cc(Br)ccc12] |
| 88 | 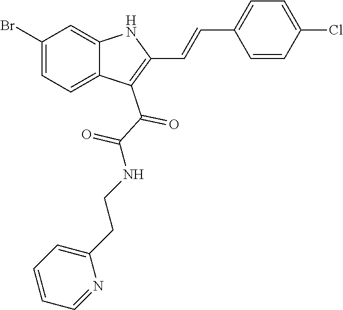 [SMILES: O=C(NCCc1ccccn1)C(=O)c1c(/C=C/c2ccc(Cl)cc2)[nH]c2cc(Br)ccc12] |
| 89 | 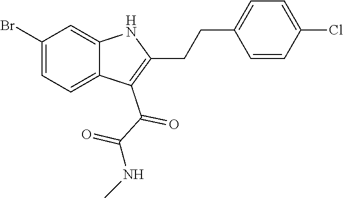 [SMILES: CNC(=O)C(=O)c1c(CCc2ccc(Cl)cc2)[nH]c2cc(Br)ccc12] |
| 90 | 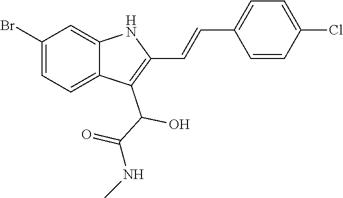 [SMILES: CNC(=O)C(O)c1c(/C=C/c2ccc(Cl)cc2)[nH]c2cc(Br)ccc12] |
| 91 | 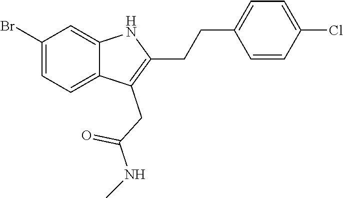 [SMILES: CNC(=O)Cc1c(CCc2ccc(Cl)cc2)[nH]c2cc(Br)ccc12] |
| 92 | 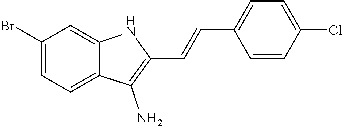 [SMILES: Nc1c(/C=C/c2ccc(Cl)cc2)[nH]c2cc(Br)ccc12] |
| 93 | 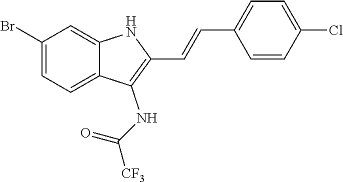 [SMILES: O=C(Nc1c(/C=C/c2ccc(Cl)cc2)[nH]c2cc(Br)ccc12)C(F)(F)F] |
| 94 | 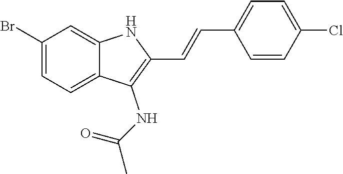 [SMILES: CC(=O)Nc1c(/C=C/c2ccc(Cl)cc2)[nH]c2cc(Br)ccc12] |
| 95 | 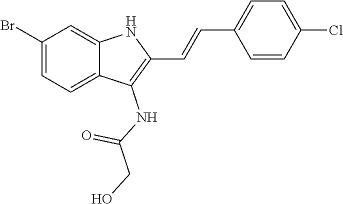 [SMILES: O=C(CO)Nc1c(/C=C/c2ccc(Cl)cc2)[nH]c2cc(Br)ccc12] |
| 96 | 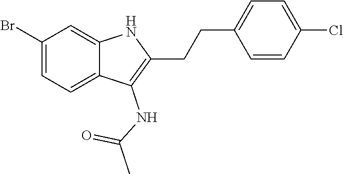 [SMILES: CC(=O)Nc1c(CCc2ccc(Cl)cc2)[nH]c2cc(Br)ccc12] |
| 97 | 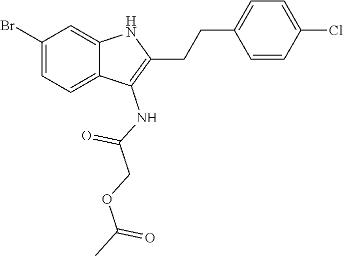 [SMILES: CC(=O)OCC(=O)Nc1c(CCc2ccc(Cl)cc2)[nH]c2cc(Br)ccc12] |
| 98 | 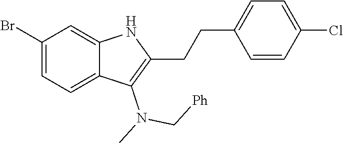 [SMILES: CN(Cc1ccccc1)c1c(CCc2ccc(Cl)cc2)[nH]c2cc(Br)ccc12] |
| 99 | 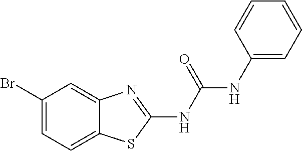 [SMILES: O=C(Nc1ccccc1)Nc1nc2cc(Br)ccc2s1] |
| 101 | 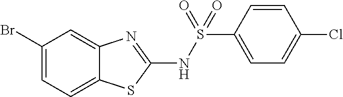 [SMILES: O=S(=O)(Nc1nc2cc(Br)ccc2s1)c1ccc(Cl)cc1] |
| 102 | 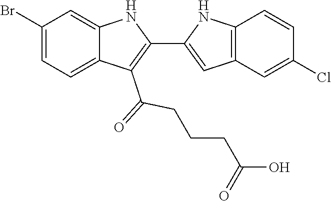 [SMILES: O=C(O)CCCC(=O)c1c(-c2cc3cc(Cl)ccc3[nH]2)[nH]c2cc(Br)ccc12] |
| 103 | 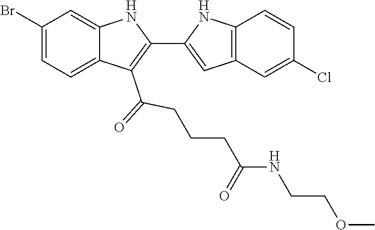 [SMILES: COCCNC(=O)CCCC(=O)c1c(-c2cc3cc(Cl)ccc3[nH]2)[nH]c2cc(Br)ccc12] |
| 104 | 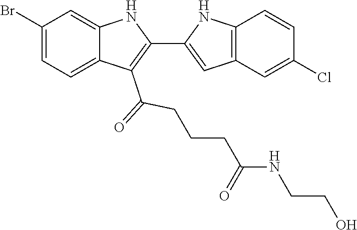 [SMILES: O=C(CCCC(=O)c1c(-c2cc3cc(Cl)ccc3[nH]2)[nH]c2cc(Br)ccc12)NCCO] |
| 105 | 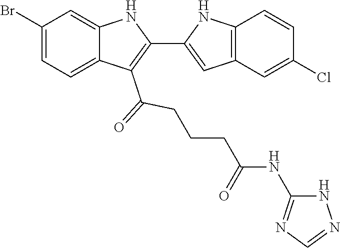 [SMILES: O=C(CCCC(=O)c1c(-c2cc3cc(Cl)ccc3[nH]2)[nH]c2cc(Br)ccc12)Nc1ncn[nH]1] |
| 106 | 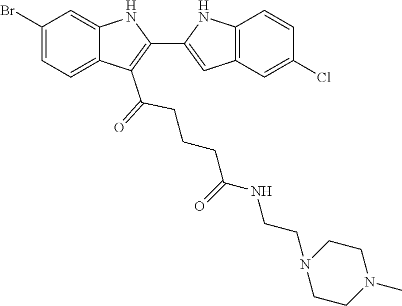 [SMILES: CN1CCN(CCNC(=O)CCCC(=O)c2c(-c3cc4cc(Cl)ccc4[nH]3)[nH]c3cc(Br)ccc23)CC1] |
| 107 | 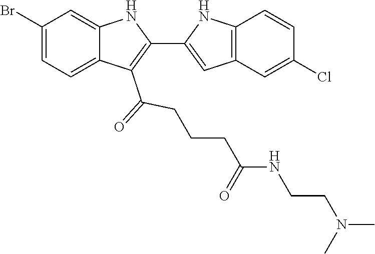 [SMILES: CN(C)CCNC(=O)CCCC(=O)c1c(-c2cc3cc(Cl)ccc3[nH]2)[nH]c2cc(Br)ccc12] |
| 108 | 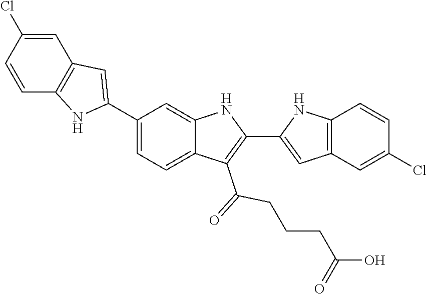 [SMILES: O=C(O)CCCC(=O)c1c(-c2cc3cc(Cl)ccc3[nH]2)[nH]c2cc(-c3cc4cc(Cl)ccc4[nH]3)ccc12] |
| 110 | 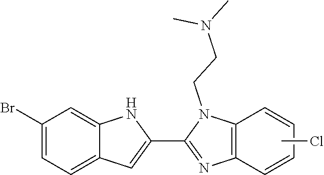 |
| 111 | 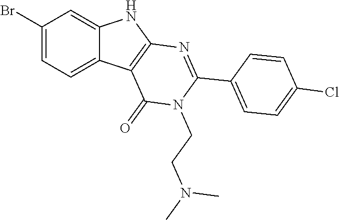 [SMILES: CN(C)CCn1c(-c2ccc(Cl)cc2)nc2[nH]c3cc(Br)ccc3c2c1=O] |
| 112 | 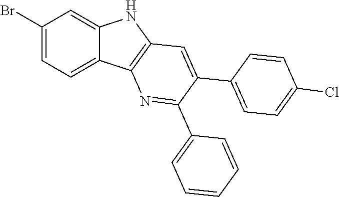 [SMILES: Clc1ccc(-c2cc3[nH]c4cc(Br)ccc4c3nc2-c2ccccc2)cc1] |
| 113 | 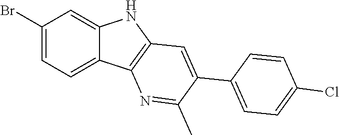 [SMILES: Cc1nc2c(cc1-c1ccc(Cl)cc1)[nH]c1cc(Br)ccc12] |
| 114 | 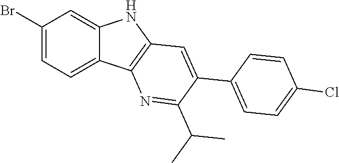 [SMILES: CC(C)c1nc2c(cc1-c1ccc(Cl)cc1)[nH]c1cc(Br)ccc12] |
| 115 | 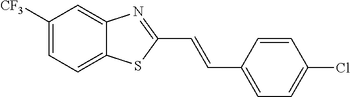 [SMILES: FC(F)(F)c1ccc2sc(/C=C/c3ccc(Cl)cc3)nc2c1] |
| 130 | 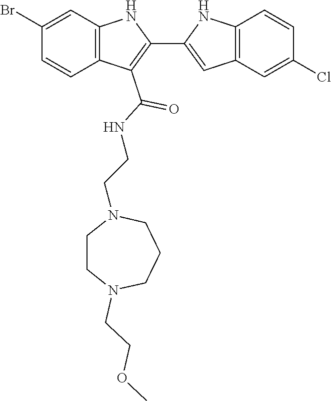 [SMILES: COCCN1CCCN(CCNC(=O)c2c(-c3cc4cc(Cl)ccc4[nH]3)[nH]c3cc(Br)ccc23)CC1] |
| 131 | 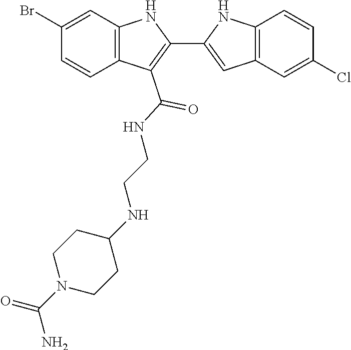 [SMILES: NC(=O)N1CCC(NCCNC(=O)c2c(-c3cc4cc(Cl)ccc4[nH]3)[nH]c3cc(Br)ccc23)CC1] |
| 132 | 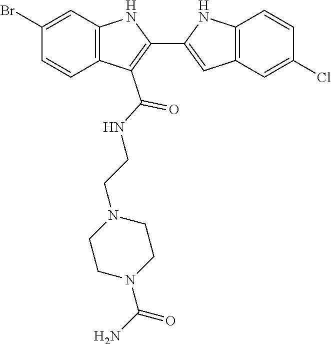 [SMILES: NC(=O)N1CCN(CCNC(=O)c2c(-c3cc4cc(Cl)ccc4[nH]3)[nH]c3cc(Br)ccc23)CC1] |
| 133 | 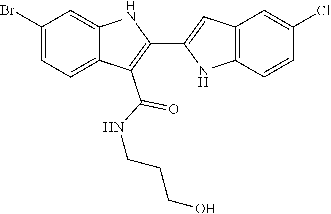 [SMILES: O=C(NCCCO)c1c(-c2cc3cc(Cl)ccc3[nH]2)[nH]c2cc(Br)ccc12] |
| 134 | 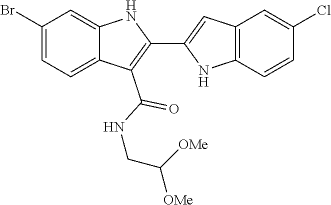 [SMILES: COC(CNC(=O)c1c(-c2cc3cc(Cl)ccc3[nH]2)[nH]c2cc(Br)ccc12)OC] |
| 135 | 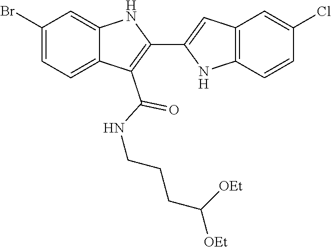 [SMILES: CCOC(CCCNC(=O)c1c(-c2cc3cc(Cl)ccc3[nH]2)[nH]c2cc(Br)ccc12)OCC] |
| 136 | 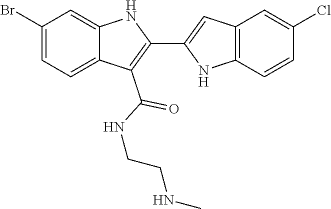 [SMILES: CNCCNC(=O)c1c(-c2cc3cc(Cl)ccc3[nH]2)[nH]c2cc(Br)ccc12] |
| 137 | 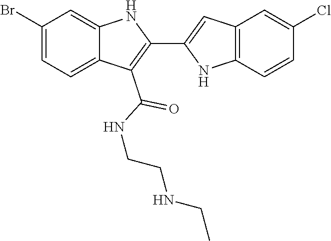 [SMILES: CCNCCNC(=O)c1c(-c2cc3cc(Cl)ccc3[nH]2)[nH]c2cc(Br)ccc12] |
| 138 | 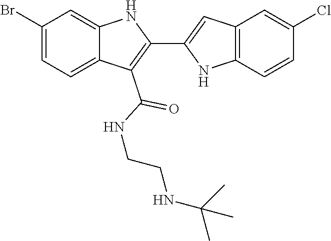 [SMILES: CC(C)(C)NCCNC(=O)c1c(-c2cc3cc(Cl)ccc3[nH]2)[nH]c2cc(Br)ccc12] |
| 139 | 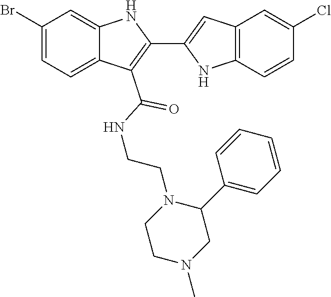 [SMILES: CN1CCN(CCNC(=O)c2c(-c3cc4cc(Cl)ccc4[nH]3)[nH]c3cc(Br)ccc23)C(c2ccccc2)C1] |
| 140 | 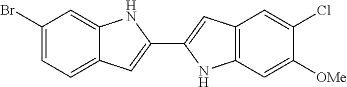 [SMILES: COc1cc2[nH]c(-c3cc4ccc(Br)cc4[nH]3)cc2cc1Cl] |
| 141 | 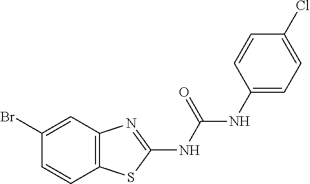 [SMILES: O=C(Nc1ccc(Cl)cc1)Nc1nc2cc(Br)ccc2s1] |
| 142 | 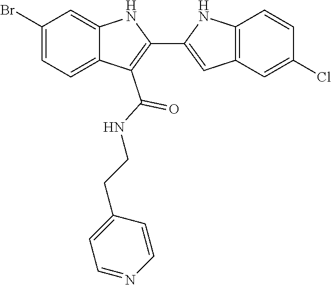 [SMILES: O=C(NCCc1ccncc1)c1c(-c2cc3cc(Cl)ccc3[nH]2)[nH]c2cc(Br)ccc12] |
| 143 | 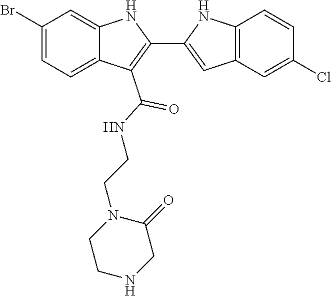 [SMILES: O=C(NCCN1CCNCC1=O)c1c(-c2cc3cc(Cl)ccc3[nH]2)[nH]c2cc(Br)ccc12] |
| 144 | 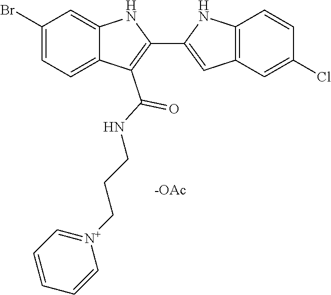 [SMILES: O=C(NCCC[n+]1ccccc1)c1c(-c2cc3cc(Cl)ccc3[nH]2)[nH]c2cc(Br)ccc12] |
| 146 | 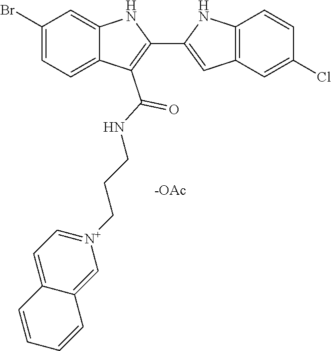 [SMILES: O=C(NCCC[n+]1ccc2ccccc2c1)c1c(-c2cc3cc(Cl)ccc3[nH]2)[nH]c2cc(Br)ccc12] |
| 148 | 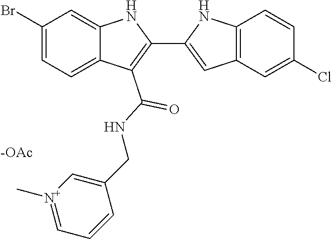 [SMILES: C[n+]1cccc(CNC(=O)c2c(-c3cc4cc(Cl)ccc4[nH]3)[nH]c3cc(Br)ccc23)c1] |
| 150 | 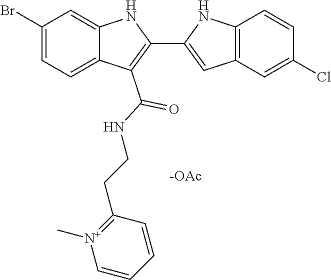 [SMILES: C[n+]1ccccc1CCNC(=O)c1c(-c2cc3cc(Cl)ccc3[nH]2)[nH]c2cc(Br)ccc12] |
| 152 | 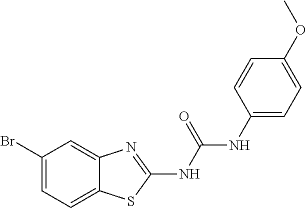 [SMILES: COc1ccc(NC(=O)Nc2nc3cc(Br)ccc3s2)cc1] |
| 154 | 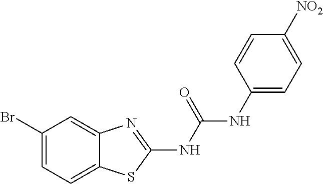 [SMILES: O=C(Nc1ccc([N+](=O)[O-])cc1)Nc1nc2cc(Br)ccc2s1] |
| 156 | 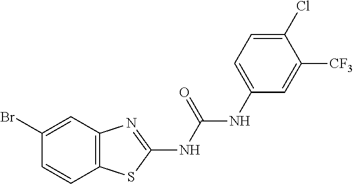 [SMILES: O=C(Nc1ccc(Cl)c(C(F)(F)F)c1)Nc1nc2cc(Br)ccc2s1] |
| 158 | 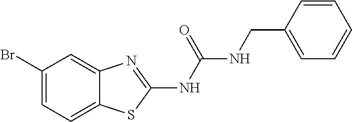 [SMILES: O=C(NCc1ccccc1)Nc1nc2cc(Br)ccc2s1] |
| 160 | 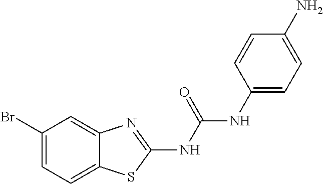 [SMILES: Nc1ccc(NC(=O)Nc2nc3cc(Br)ccc3s2)cc1] |
| 166 | 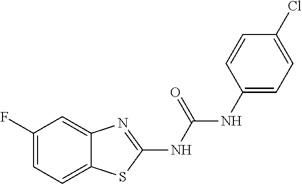 [SMILES: O=C(Nc1ccc(Cl)cc1)Nc1nc2cc(F)ccc2s1] |
| 168 | 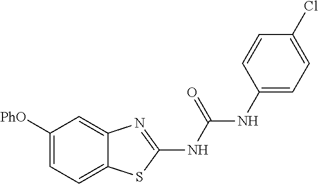 [SMILES: O=C(Nc1ccc(Cl)cc1)Nc1nc2cc(Oc3ccccc3)ccc2s1] |
| 170 | 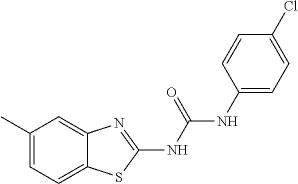 [SMILES: Cc1ccc2sc(NC(=O)Nc3ccc(Cl)cc3)nc2c1] |
| 172 | 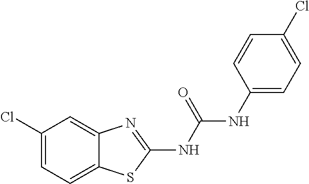 [SMILES: O=C(Nc1ccc(Cl)cc1)Nc1nc2cc(Cl)ccc2s1] |
| 174 | 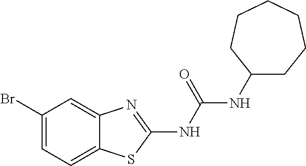 [SMILES: O=C(Nc1nc2cc(Br)ccc2s1)NC1CCCCCC1] |
| 180 | 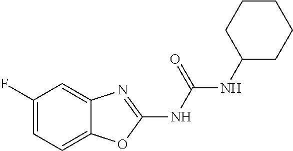 [SMILES: O=C(Nc1nc2cc(F)ccc2o1)NC1CCCCC1] |
| 182 | 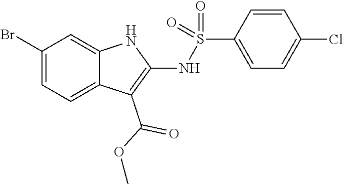 [SMILES: COC(=O)c1c(NS(=O)(=O)c2ccc(Cl)cc2)[nH]c2cc(Br)ccc12] |
| 184 | 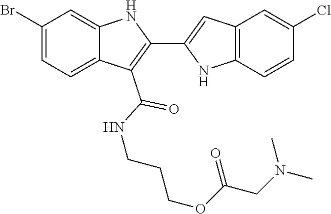 [SMILES: CN(C)CC(=O)OCCCNC(=O)c1c(-c2cc3cc(Cl)ccc3[nH]2)[nH]c2cc(Br)ccc12] |
| 186 | 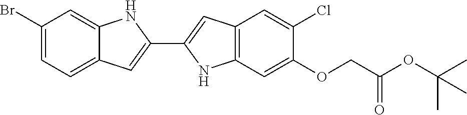 [SMILES: CC(C)(C)OC(=O)COc1cc2[nH]c(-c3cc4ccc(Br)cc4[nH]3)cc2cc1Cl] |
| 188 | 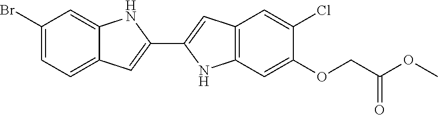 [SMILES: COC(=O)COc1cc2[nH]c(-c3cc4ccc(Br)cc4[nH]3)cc2cc1Cl] |
| 190 | 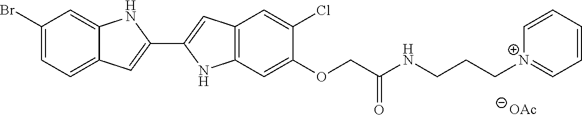 [SMILES: O=C(COc1cc2[nH]c(-c3cc4ccc(Br)cc4[nH]3)cc2cc1Cl)NCCC[n+]1ccccc1 ; "OAc"] |
| 192 | 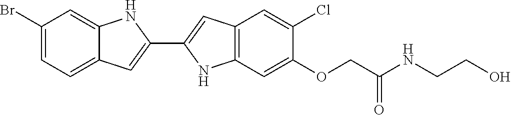 [SMILES: O=C(COc1cc2[nH]c(-c3cc4ccc(Br)cc4[nH]3)cc2cc1Cl)NCCO] |
| 194 | 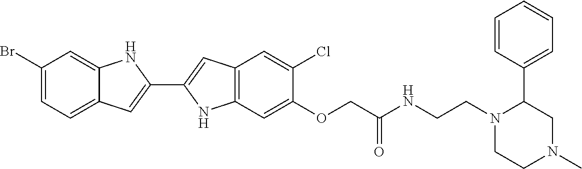 [SMILES: CN1CCN(CCNC(=O)COc2cc3[nH]c(-c4cc5ccc(Br)cc5[nH]4)cc3cc2Cl)C(c2ccccc2)C1] |
| 196 | 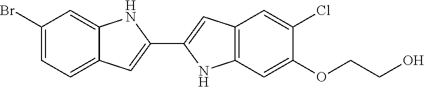 [SMILES: OCCOc1cc2[nH]c(-c3cc4ccc(Br)cc4[nH]3)cc2cc1Cl] |
| 198 | 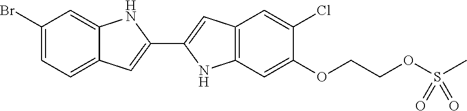 [SMILES: CS(=O)(=O)OCCOc1cc2[nH]c(-c3cc4ccc(Br)cc4[nH]3)cc2cc1Cl] |
| 200 | 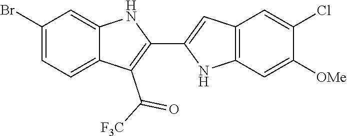 [SMILES: COc1cc2[nH]c(-c3[nH]c4cc(Br)ccc4c3C(=O)C(F)(F)F)cc2cc1Cl] |
| 202 | 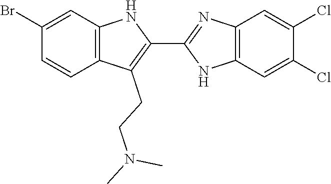 [SMILES: CN(C)CCc1c(-c2nc3cc(Cl)c(Cl)cc3[nH]2)[nH]c2cc(Br)ccc12] |
| 204 | 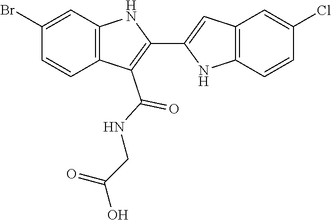 [SMILES: O=C(O)CNC(=O)c1c(-c2cc3cc(Cl)ccc3[nH]2)[nH]c2cc(Br)ccc12] |
| 206 | 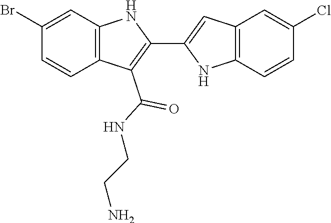 [SMILES: NCCNC(=O)c1c(-c2cc3cc(Cl)ccc3[nH]2)[nH]c2cc(Br)ccc12] |
| 208 | 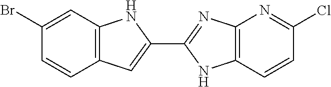 [SMILES: Clc1ccc2[nH]c(-c3cc4ccc(Br)cc4[nH]3)nc2n1] |
| 210 | 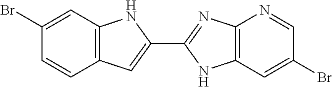 [SMILES: Brc1ccc2cc(-c3nc4ncc(Br)cc4[nH]3)[nH]c2c1] |
| 212 | 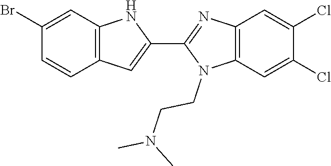 [SMILES: CN(C)CCn1c(-c2cc3ccc(Br)cc3[nH]2)nc2cc(Cl)c(Cl)cc21] |
| 214 | 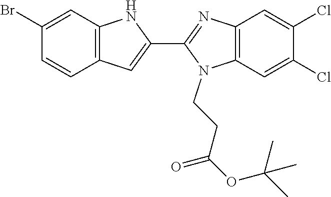 [SMILES: CC(C)(C)OC(=O)CCn1c(-c2cc3ccc(Br)cc3[nH]2)nc2cc(Cl)c(Cl)cc21] |
| 216 | 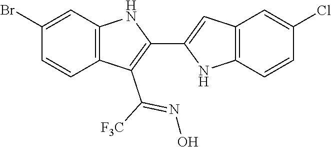 [SMILES: O/N=C(/c1c(-c2cc3cc(Cl)ccc3[nH]2)[nH]c2cc(Br)ccc12)C(F)(F)F] |
| 218 | 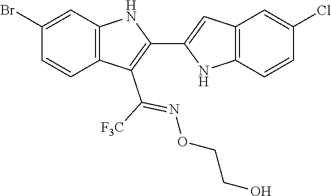 [SMILES: OCCO/N=C(/c1c(-c2cc3cc(Cl)ccc3[nH]2)[nH]c2cc(Br)ccc12)C(F)(F)F] |
| 220 | 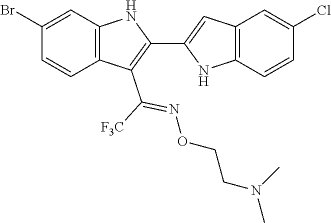 [SMILES: CN(C)CCO/N=C(/c1c(-c2cc3cc(Cl)ccc3[nH]2)[nH]c2cc(Br)ccc12)C(F)(F)F] |
| 222 | 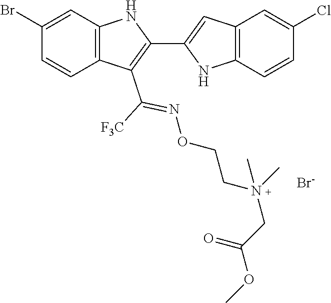 |
| 224 | 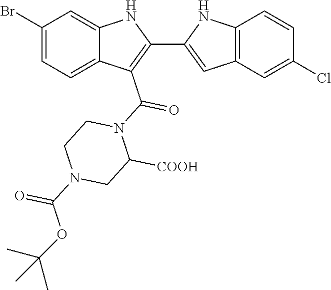 [SMILES: CC(C)(C)OC(=O)N1CCN(C(=O)c2c(-c3cc4cc(Cl)ccc4[nH]3)[nH]c3cc(Br)ccc23)C(C(=O)O)C1] |
| 226 | 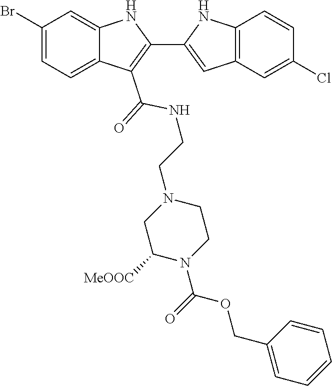 [SMILES: COC(=O)[C@@H]1CN(CCNC(=O)c2c(-c3cc4cc(Cl)ccc4[nH]3)[nH]c3cc(Br)ccc23)CCN1C(=O)OCc1ccccc1] |
| 228 | 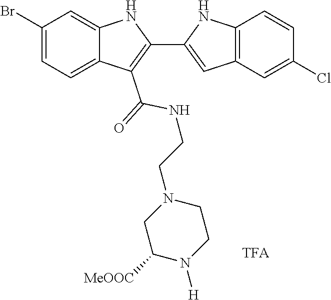 [SMILES: COC(=O)[C@@H]1CN(CCNC(=O)c2c(-c3cc4cc(Cl)ccc4[nH]3)[nH]c3cc(Br)ccc23)CCN1] |

TABLE 2

| Compound | |
|---|---|
| # | Chemical Structure |
| 1 | 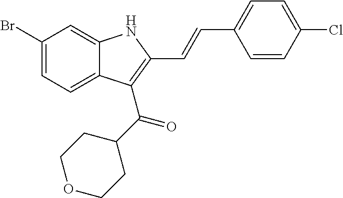 [SMILES: O=C(c1c(/C=C/c2ccc(Cl)cc2)[nH]c2cc(Br)ccc12)C1CCOCC1] |
| 2 | 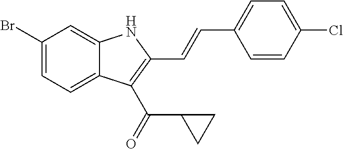 [SMILES: O=C(c1c(/C=C/c2ccc(Cl)cc2)[nH]c2cc(Br)ccc12)C1CC1] |
| 3 | 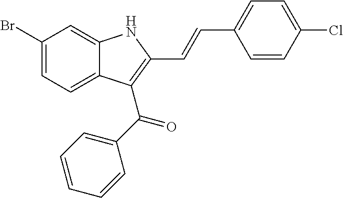 [SMILES: O=C(c1ccccc1)c1c(/C=C/c2ccc(Cl)cc2)[nH]c2cc(Br)ccc12] |
| 4 | 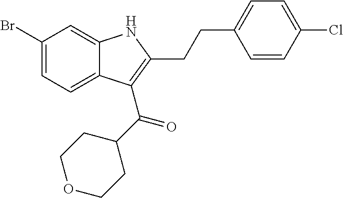 [SMILES: O=C(c1c(CCc2ccc(Cl)cc2)[nH]c2cc(Br)ccc12)C1CCOCC1] |
| 5 | 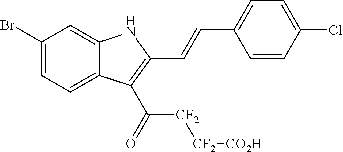 [SMILES: O=C(O)C(F)(F)C(F)(F)C(=O)c1c(/C=C/c2ccc(Cl)cc2)[nH]c2cc(Br)ccc12] |
| 6 | 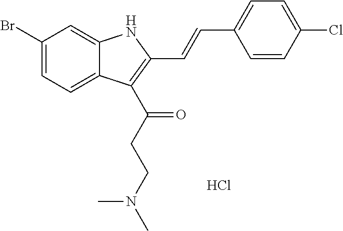 [SMILES: CN(C)CCC(=O)c1c(/C=C/c2ccc(Cl)cc2)[nH]c2cc(Br)ccc12 ; Cl] |
| 7 | 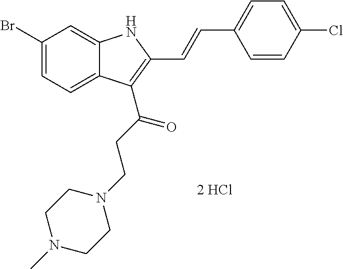 [SMILES: CN1CCN(CCC(=O)c2c(/C=C/c3ccc(Cl)cc3)[nH]c3cc(Br)ccc23)CC1] |
| 8 | 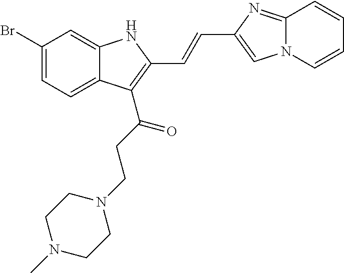 [SMILES: CN1CCN(CCC(=O)c2c(/C=C/c3cn4ccccc4n3)[nH]c3cc(Br)ccc23)CC1] |
| 9 | 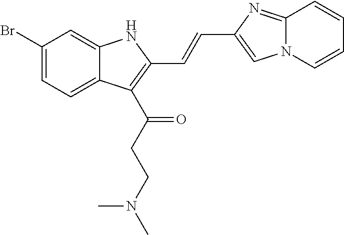 [SMILES: CN(C)CCC(=O)c1c(/C=C/c2cn3ccccc3n2)[nH]c2cc(Br)ccc12] |
| 10 | 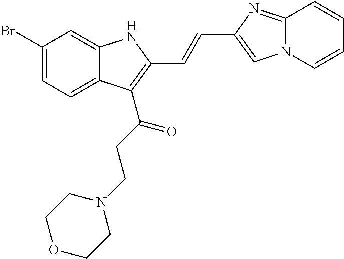 [SMILES: O=C(CCN1CCOCC1)c1c(/C=C/c2cn3ccccc3n2)[nH]c2cc(Br)ccc12] |
| 11 | 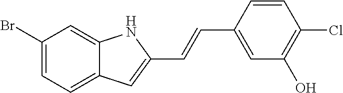 [SMILES: Oc1cc(/C=C/c2cc3ccc(Br)cc3[nH]2)ccc1Cl] |
| 12 | 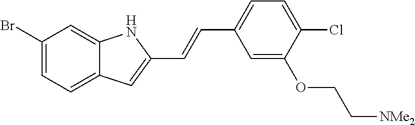 [SMILES: CN(C)CCOc1cc(/C=C/c2cc3ccc(Br)cc3[nH]2)ccc1Cl] |
| 13 | 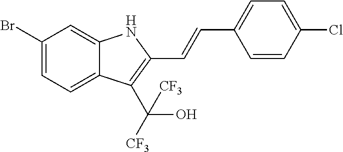 [SMILES: OC(c1c(/C=C/c2ccc(Cl)cc2)[nH]c2cc(Br)ccc12)(C(F)(F)F)C(F)(F)F] |
| 14 | 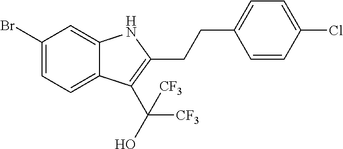 [SMILES: OC(c1c(CCc2ccc(Cl)cc2)[nH]c2cc(Br)ccc12)(C(F)(F)F)C(F)(F)F] |
| 15 | 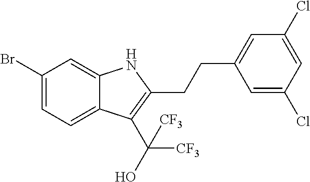 [SMILES: OC(c1c(CCc2cc(Cl)cc(Cl)c2)[nH]c2cc(Br)ccc12)(C(F)(F)F)C(F)(F)F] |
| 16 | 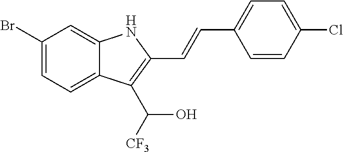 [SMILES: OC(c1c(/C=C/c2ccc(Cl)cc2)[nH]c2cc(Br)ccc12)C(F)(F)F] |
| 17 | 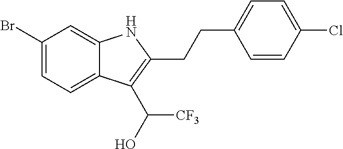 [SMILES: OC(c1c(CCc2ccc(Cl)cc2)[nH]c2cc(Br)ccc12)C(F)(F)F] |
| 18 | 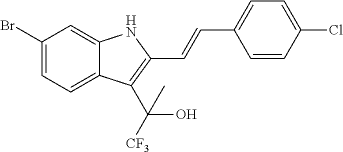 [SMILES: CC(O)(c1c(/C=C/c2ccc(Cl)cc2)[nH]c2cc(Br)ccc12)C(F)(F)F] |
| 19 | 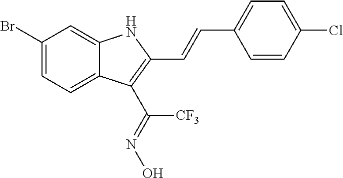 [SMILES: O/N=C(/c1c(/C=C/c2ccc(Cl)cc2)[nH]c2cc(Br)ccc12)C(F)(F)F] |
| 20 | 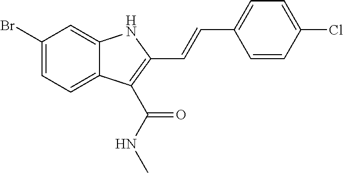 [SMILES: CNC(=O)c1c(/C=C/c2ccc(Cl)cc2)[nH]c2cc(Br)ccc12] |
| 21 | 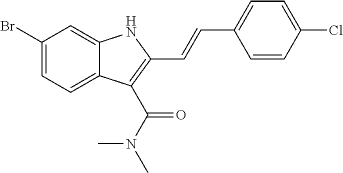 [SMILES: CN(C)C(=O)c1c(/C=C/c2ccc(Cl)cc2)[nH]c2cc(Br)ccc12] |
| 22 | 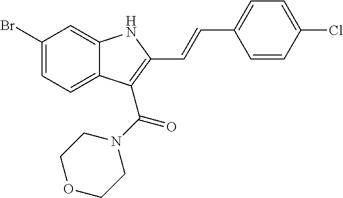 [SMILES: O=C(c1c(/C=C/c2ccc(Cl)cc2)[nH]c2cc(Br)ccc12)N1CCOCC1] |
| 23 | 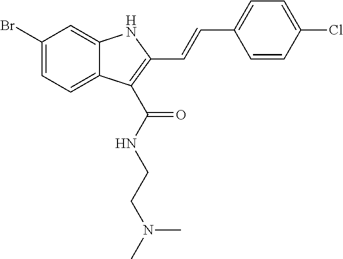 [SMILES: CN(C)CCNC(=O)c1c(/C=C/c2ccc(Cl)cc2)[nH]c2cc(Br)ccc12] |
| 24 | 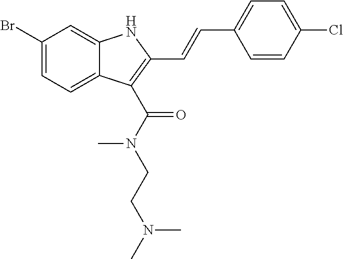 [SMILES: CN(C)CCN(C)C(=O)c1c(/C=C/c2ccc(Cl)cc2)[nH]c2cc(Br)ccc12] |
| 25 | 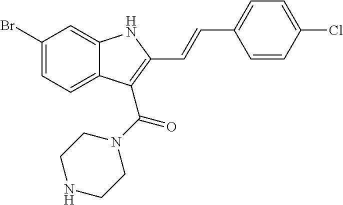 [SMILES: O=C(c1c(/C=C/c2ccc(Cl)cc2)[nH]c2cc(Br)ccc12)N1CCNCC1] |
| 26 | 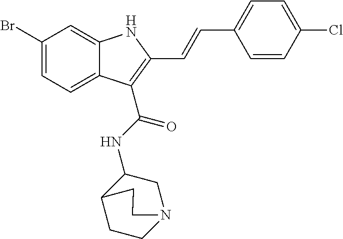 [SMILES: O=C(NC1CN2CCC1CC2)c1c(/C=C/c2ccc(Cl)cc2)[nH]c2cc(Br)ccc12] |
| 27 | 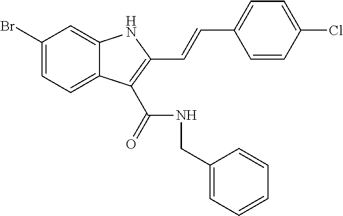 [SMILES: O=C(NCc1ccccc1)c1c(/C=C/c2ccc(Cl)cc2)[nH]c2cc(Br)ccc12] |
| 28 | 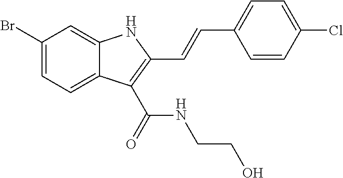 [SMILES: O=C(NCCO)c1c(/C=C/c2ccc(Cl)cc2)[nH]c2cc(Br)ccc12] |
| 29 | 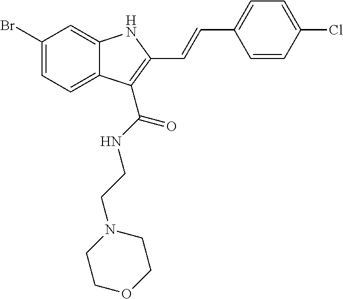 [SMILES: O=C(NCCN1CCOCC1)c1c(/C=C/c2ccc(Cl)cc2)[nH]c2cc(Br)ccc12] |
| 30 | 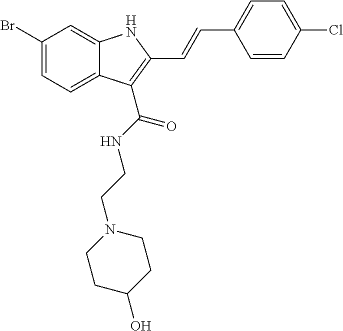 [SMILES: O=C(NCCN1CCC(O)CC1)c1c(/C=C/c2ccc(Cl)cc2)[nH]c2cc(Br)ccc12] |
| 31 | 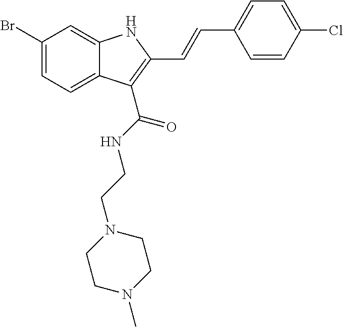 [SMILES: CN1CCN(CCNC(=O)c2c(/C=C/c3ccc(Cl)cc3)[nH]c3cc(Br)ccc23)CC1] |
| 32 | 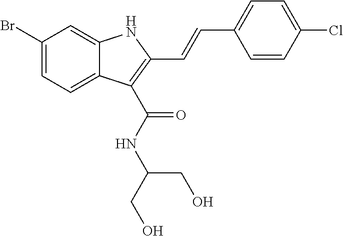 [SMILES: O=C(NC(CO)CO)c1c(/C=C/c2ccc(Cl)cc2)[nH]c2cc(Br)ccc12] |
| 33 | 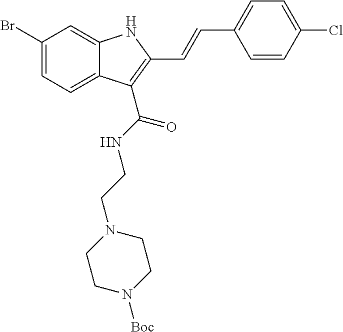 [SMILES: CC(C)(C)OC(=O)N1CCN(CCNC(=O)c2c(/C=C/c3ccc(Cl)cc3)[nH]c3cc(Br)ccc23)CC1] |
| 34 | 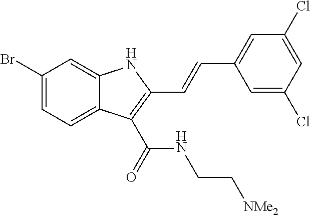 [SMILES: CN(C)CCNC(=O)c1c(/C=C/c2cc(Cl)cc(Cl)c2)[nH]c2cc(Br)ccc12] |
| 35 | 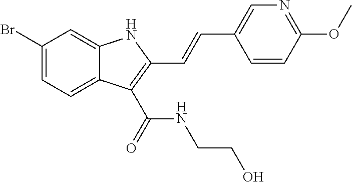 [SMILES: COc1ccc(/C=C/c2[nH]c3cc(Br)ccc3c2C(=O)NCCO)cn1] |
| 36 | 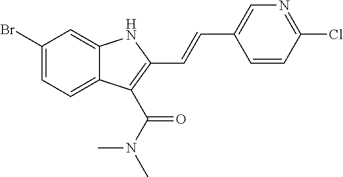 [SMILES: CN(C)C(=O)c1c(/C=C/c2ccc(Cl)nc2)[nH]c2cc(Br)ccc12] |
| 37 | 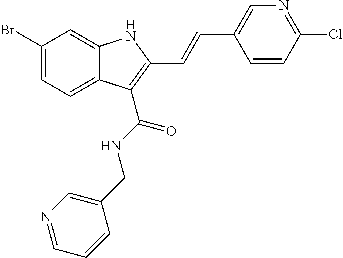 [SMILES: O=C(NCc1cccnc1)c1c(/C=C/c2ccc(Cl)nc2)[nH]c2cc(Br)ccc12] |
| 38 | 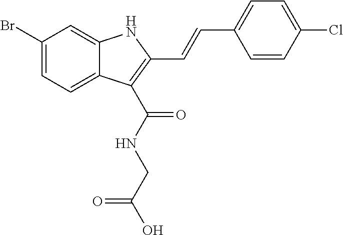 [SMILES: O=C(O)CNC(=O)c1c(/C=C/c2ccc(Cl)cc2)[nH]c2cc(Br)ccc12] |
| 39 | 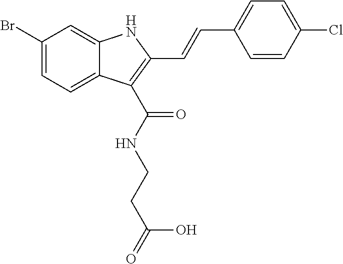 [SMILES: O=C(O)CCNC(=O)c1c(/C=C/c2ccc(Cl)cc2)[nH]c2cc(Br)ccc12] |
| 40 | 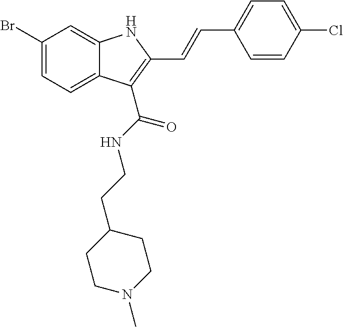 [SMILES: CN1CCC(CCNC(=O)c2c(/C=C/c3ccc(Cl)cc3)[nH]c3cc(Br)ccc23)CC1] |
| 41 | 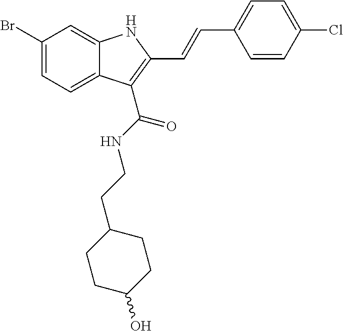 [SMILES: O=C(NCCC1CCC(O)CC1)c1c(/C=C/c2ccc(Cl)cc2)[nH]c2cc(Br)ccc12] |
| 42 | 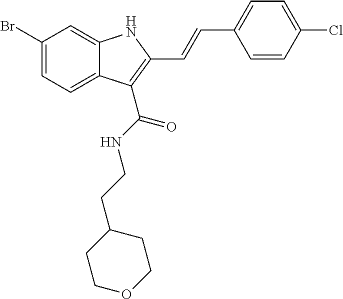 [SMILES: O=C(NCCC1CCOCC1)c1c(/C=C/c2ccc(Cl)cc2)[nH]c2cc(Br)ccc12] |
| 43 | 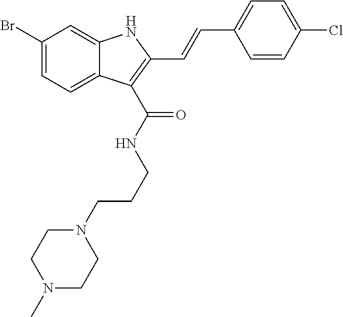 [SMILES: CN1CCN(CCCNC(=O)c2c(/C=C/c3ccc(Cl)cc3)[nH]c3cc(Br)ccc23)CC1] |
| 44 | 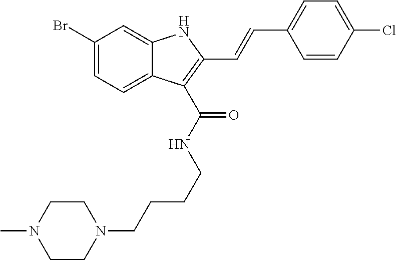 [SMILES: CN1CCN(CCCCNC(=O)c2c(/C=C/c3ccc(Cl)cc3)[nH]c3cc(Br)ccc23)CC1] |
| 45 | 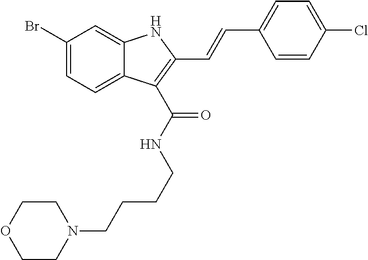 [SMILES: O=C(NCCCCN1CCOCC1)c1c(/C=C/c2ccc(Cl)cc2)[nH]c2cc(Br)ccc12] |
| 46 | 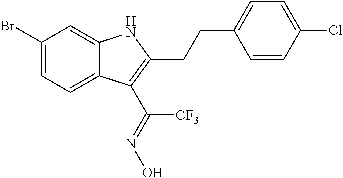 [SMILES: O/N=C(/c1c(CCc2ccc(Cl)cc2)[nH]c2cc(Br)ccc12)C(F)(F)F] |
| 47 | 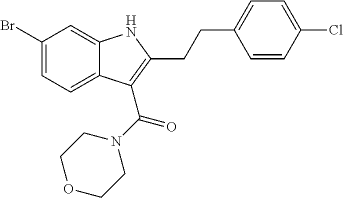 [SMILES: O=C(c1c(CCc2ccc(Cl)cc2)[nH]c2cc(Br)ccc12)N1CCOCC1] |
| 48 | 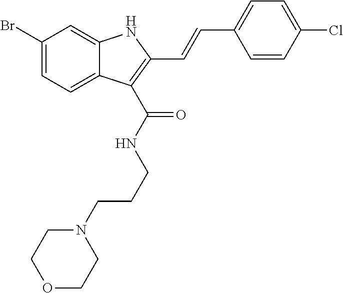 [SMILES: O=C(NCCCN1CCOCC1)c1c(/C=C/c2ccc(Cl)cc2)[nH]c2cc(Br)ccc12] |
| 49 | 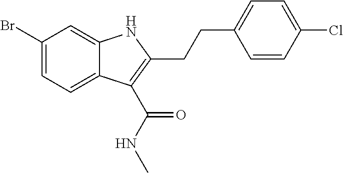 [SMILES: CNC(=O)c1c(CCc2ccc(Cl)cc2)[nH]c2cc(Br)ccc12] |
| 50 | 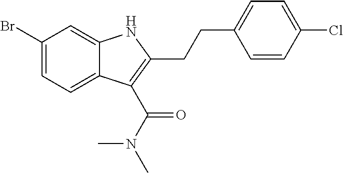 [SMILES: CN(C)C(=O)c1c(CCc2ccc(Cl)cc2)[nH]c2cc(Br)ccc12] |
| 51 | 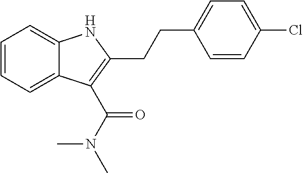 [SMILES: CN(C)C(=O)c1c(CCc2ccc(Cl)cc2)[nH]c2ccccc12] |
| 52 | 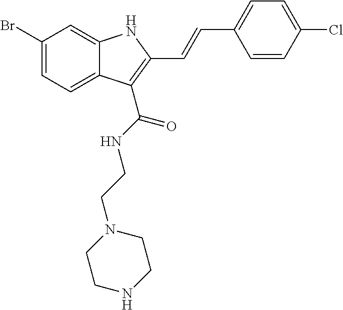 [SMILES: O=C(NCCN1CCNCC1)c1c(/C=C/c2ccc(Cl)cc2)[nH]c2cc(Br)ccc12] |
| 53 | 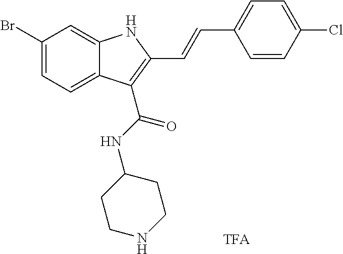 [SMILES: O=C(NC1CCNCC1)c1c(/C=C/c2ccc(Cl)cc2)[nH]c2cc(Br)ccc12] |
| 54 | 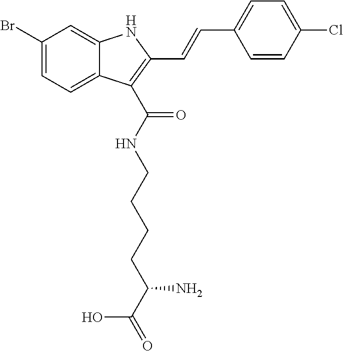 [SMILES: N[C@@H](CCCCNC(=O)c1c(/C=C/c2ccc(Cl)cc2)[nH]c2cc(Br)ccc12)C(=O)O] |
| 55 | 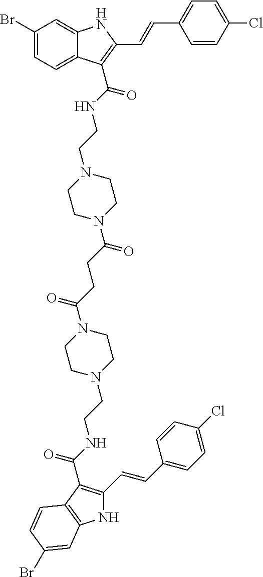 [SMILES: O=C(NCCN1CCN(C(=O)CCC(=O)N2CCN(CCNC(=O)c3c(/C=C/c4ccc(Cl)cc4)[nH]c4cc(Br)ccc34)CC2)CC1)c1c(/C=C/c2ccc(Cl)cc2)[nH]c2cc(Br)ccc12] |
| 56 | 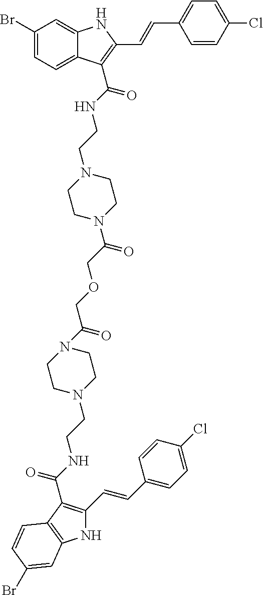 [SMILES: O=C(NCCN1CCN(C(=O)COCC(=O)N2CCN(CCNC(=O)c3c(/C=C/c4ccc(Cl)cc4)[nH]c4cc(Br)ccc34)CC2)CC1)c1c(/C=C/c2ccc(Cl)cc2)[nH]c2cc(Br)ccc12] |
| 57 | 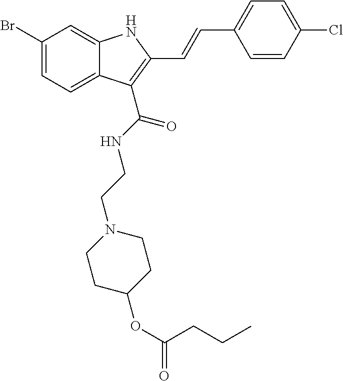 [SMILES: CCCC(=O)OC1CCN(CCNC(=O)c2c(/C=C/c3ccc(Cl)cc3)[nH]c3cc(Br)ccc23)CC1] |
| 58 | 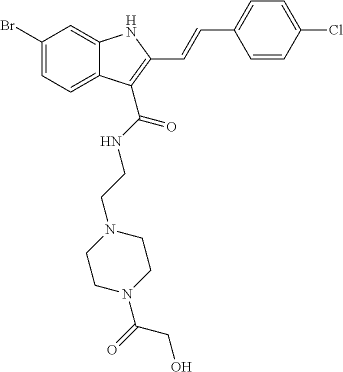 [SMILES: O=C(NCCN1CCN(C(=O)CO)CC1)c1c(/C=C/c2ccc(Cl)cc2)[nH]c2cc(Br)ccc12] |
| 59 | 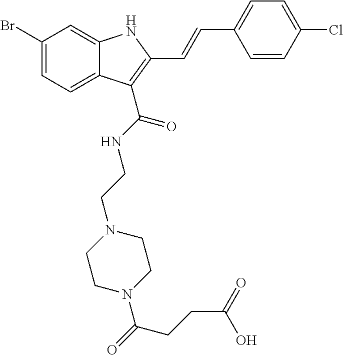 [SMILES: O=C(O)CCC(=O)N1CCN(CCNC(=O)c2c(/C=C/c3ccc(Cl)cc3)[nH]c3cc(Br)ccc23)CC1] |
| 60 | 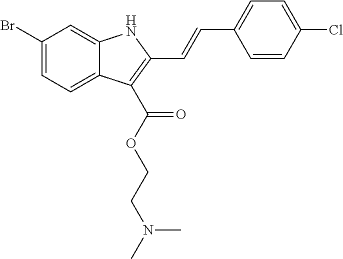 [SMILES: CN(C)CCOC(=O)c1c(/C=C/c2ccc(Cl)cc2)[nH]c2cc(Br)ccc12] |
| 61 | 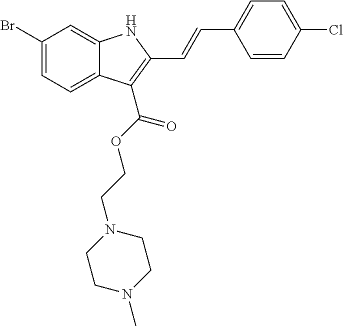 [SMILES: CN1CCN(CCOC(=O)c2c(/C=C/c3ccc(Cl)cc3)[nH]c3cc(Br)ccc23)CC1] |
| 62 | 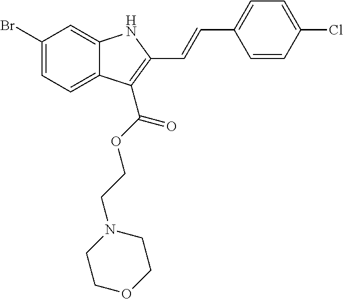 [SMILES: O=C(OCCN1CCOCC1)c1c(/C=C/c2ccc(Cl)cc2)[nH]c2cc(Br)ccc12] |
| 63 | 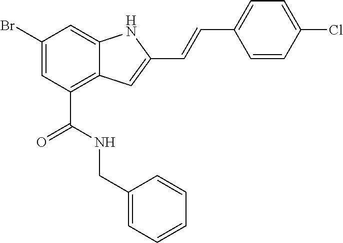 [SMILES: O=C(NCc1ccccc1)c1cc(Br)cc2[nH]c(/C=C/c3ccc(Cl)cc3)cc12] |
| 64 | 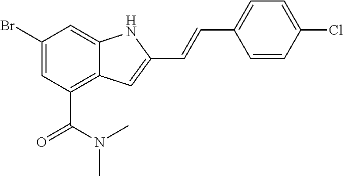 [SMILES: CN(C)C(=O)c1cc(Br)cc2[nH]c(/C=C/c3ccc(Cl)cc3)cc12] |
| 65 | 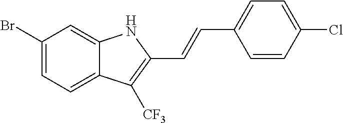 [SMILES: FC(F)(F)c1c(/C=C/c2ccc(Cl)cc2)[nH]c2cc(Br)ccc12] |
| 66 | 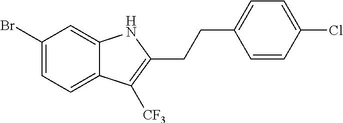 [SMILES: FC(F)(F)c1c(CCc2ccc(Cl)cc2)[nH]c2cc(Br)ccc12] |
| 67 | 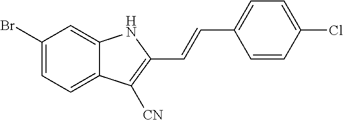 [SMILES: N#Cc1c(/C=C/c2ccc(Cl)cc2)[nH]c2cc(Br)ccc12] |
| 68 | 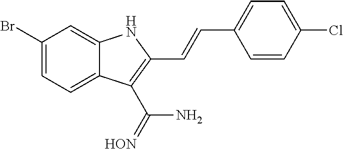 |
| 69 | 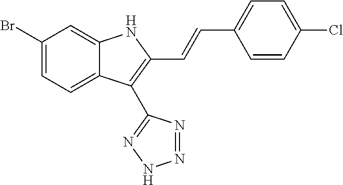 [SMILES: Clc1ccc(/C=C/c2[nH]c3cc(Br)ccc3c2-c2nn[nH]n2)cc1] |
| 70 | 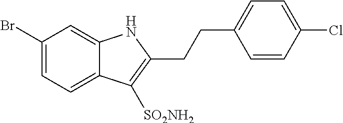 [SMILES: NS(=O)(=O)c1c(CCc2ccc(Cl)cc2)[nH]c2cc(Br)ccc12] |
| 71 | 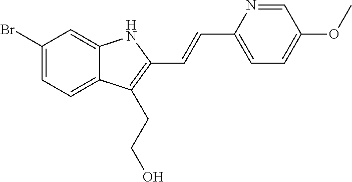 [SMILES: COc1ccc(/C=C/c2[nH]c3cc(Br)ccc3c2CCO)nc1] |
| 72 | 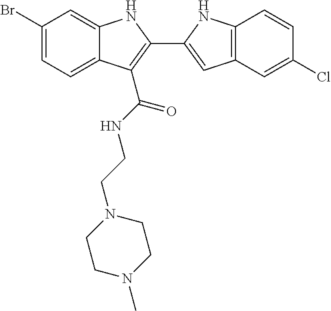 [SMILES: CN1CCN(CCNC(=O)c2c(-c3cc4cc(Cl)ccc4[nH]3)[nH]c3cc(Br)ccc23)CC1] |
| 73 | 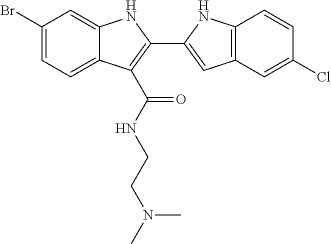 [SMILES: CN(C)CCNC(=O)c1c(-c2cc3cc(Cl)ccc3[nH]2)[nH]c2cc(Br)ccc12] |
| 74 | 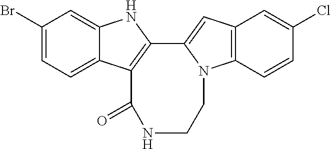 [SMILES: O=C1NCCn2c(cc3cc(Cl)ccc32)-c2[nH]c3cc(Br)ccc3c21] |
| 75 | 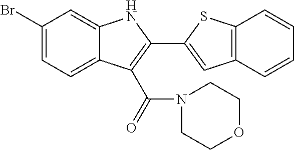 [SMILES: O=C(c1c(-c2cc3ccccc3s2)[nH]c2cc(Br)ccc12)N1CCOCC1] |
| 76 | 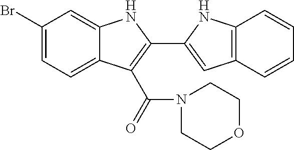 [SMILES: O=C(c1c(-c2cc3ccccc3[nH]2)[nH]c2cc(Br)ccc12)N1CCOCC1] |
| 77 | 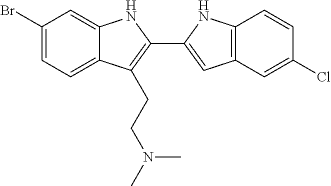 [SMILES: CN(C)CCc1c(-c2cc3cc(Cl)ccc3[nH]2)[nH]c2cc(Br)ccc12] |
| 78 | 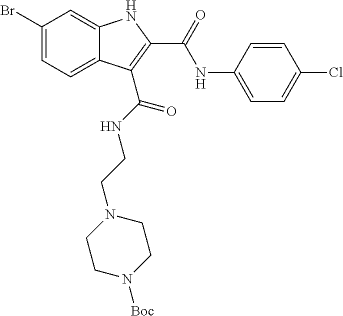 [SMILES: CC(C)(C)OC(=O)N1CCN(CCNC(=O)c2c(C(=O)Nc3ccc(Cl)cc3)[nH]c3cc(Br)ccc23)CC1] |
| 79 | 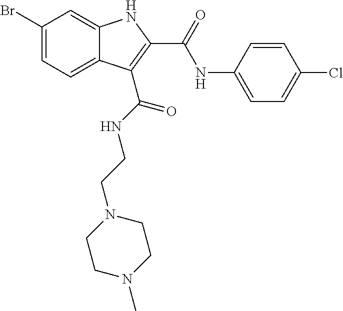 [SMILES: CN1CCN(CCNC(=O)c2c(C(=O)Nc3ccc(Cl)cc3)[nH]c3cc(Br)ccc23)CC1] |
| 80 | 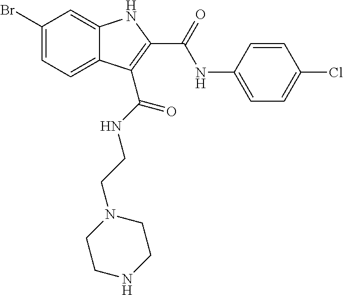 [SMILES: O=C(Nc1ccc(Cl)cc1)c1[nH]c2cc(Br)ccc2c1C(=O)NCCN1CCNCC1] |
| 81 | 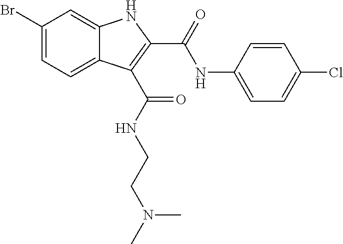 [SMILES: CN(C)CCNC(=O)c1c(C(=O)Nc2ccc(Cl)cc2)[nH]c2cc(Br)ccc12] |
| 82 | 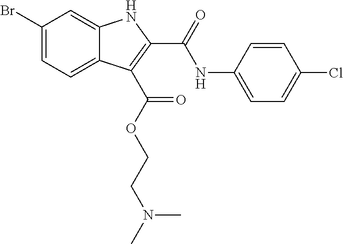 [SMILES: CN(C)CCOC(=O)c1c(C(=O)Nc2ccc(Cl)cc2)[nH]c2cc(Br)ccc12] |
| 83 | 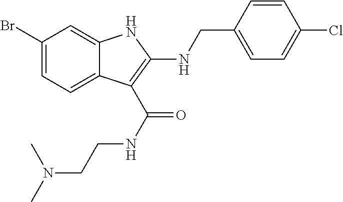 [SMILES: CN(C)CCNC(=O)c1c(NCc2ccc(Cl)cc2)[nH]c2cc(Br)ccc12] |
| 84 | 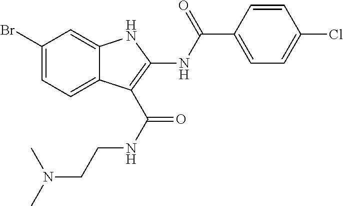 [SMILES: CN(C)CCNC(=O)c1c(NC(=O)c2ccc(Cl)cc2)[nH]c2cc(Br)ccc12] |
| 85 | 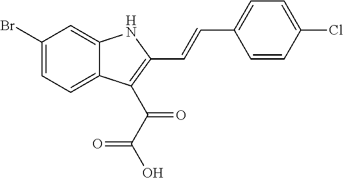 [SMILES: O=C(O)C(=O)c1c(/C=C/c2ccc(Cl)cc2)[nH]c2cc(Br)ccc12] |
| 86 | 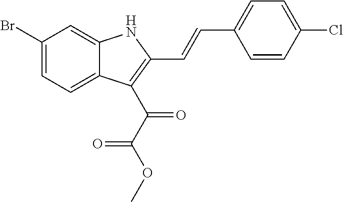 [SMILES: COC(=O)C(=O)c1c(/C=C/c2ccc(Cl)cc2)[nH]c2cc(Br)ccc12] |
| 87 | 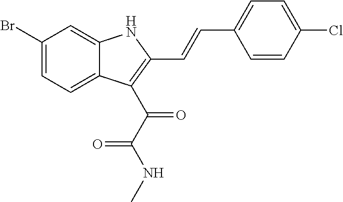 [SMILES: CNC(=O)C(=O)c1c(/C=C/c2ccc(Cl)cc2)[nH]c2cc(Br)ccc12] |
| 88 | 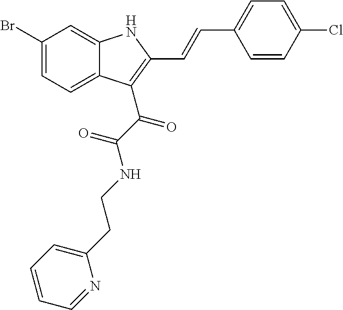 [SMILES: O=C(NCCc1ccccn1)C(=O)c1c(/C=C/c2ccc(Cl)cc2)[nH]c2cc(Br)ccc12] |
| 89 | 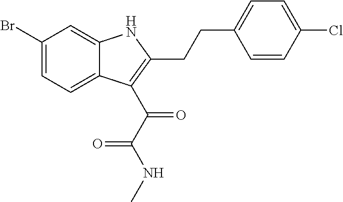 [SMILES: CNC(=O)C(=O)c1c(CCc2ccc(Cl)cc2)[nH]c2cc(Br)ccc12] |
| 90 | 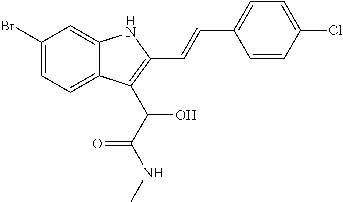 [SMILES: CNC(=O)C(O)c1c(/C=C/c2ccc(Cl)cc2)[nH]c2cc(Br)ccc12] |
| 91 | 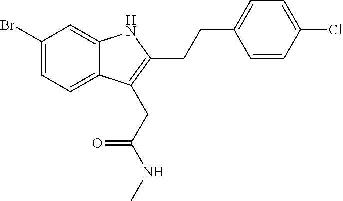 [SMILES: CNC(=O)Cc1c(CCc2ccc(Cl)cc2)[nH]c2cc(Br)ccc12] |
| 92 | 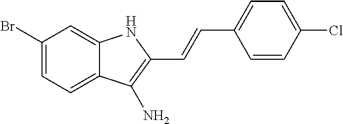 [SMILES: Nc1c(/C=C/c2ccc(Cl)cc2)[nH]c2cc(Br)ccc12] |
| 93 | 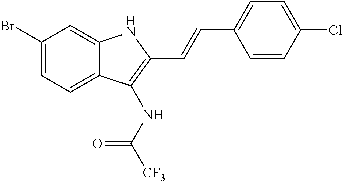 [SMILES: O=C(Nc1c(/C=C/c2ccc(Cl)cc2)[nH]c2cc(Br)ccc12)C(F)(F)F] |
| 94 | 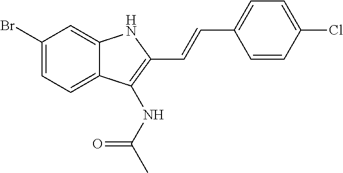 [SMILES: CC(=O)Nc1c(/C=C/c2ccc(Cl)cc2)[nH]c2cc(Br)ccc12] |
| 95 | 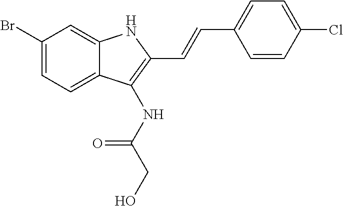 [SMILES: O=C(CO)Nc1c(/C=C/c2ccc(Cl)cc2)[nH]c2cc(Br)ccc12] |
| 96 | 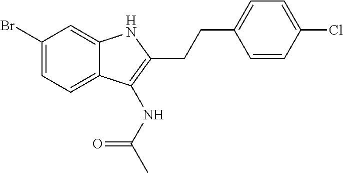 [SMILES: CC(=O)Nc1c(CCc2ccc(Cl)cc2)[nH]c2cc(Br)ccc12] |
| 97 | 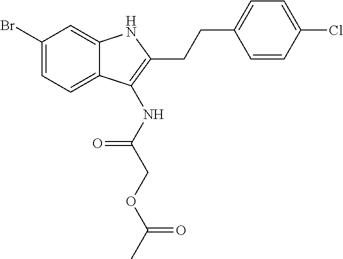 [SMILES: CC(=O)OCC(=O)Nc1c(CCc2ccc(Cl)cc2)[nH]c2cc(Br)ccc12] |
| 98 | 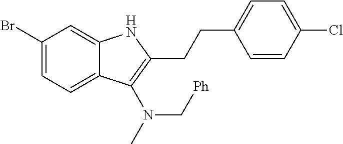 [SMILES: CN(Cc1ccccc1)c1c(CCc2ccc(Cl)cc2)[nH]c2cc(Br)ccc12] |
| 99 | 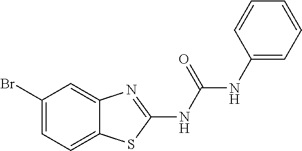 [SMILES: O=C(Nc1ccccc1)Nc1nc2cc(Br)ccc2s1] |
| 100 | 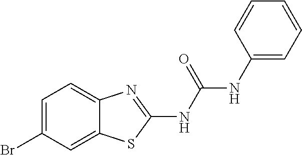 [SMILES: O=C(Nc1ccccc1)Nc1nc2ccc(Br)cc2s1] |
| 101 | 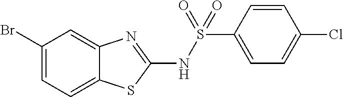 [SMILES: O=S(=O)(Nc1nc2cc(Br)ccc2s1)c1ccc(Cl)cc1] |
| 102 | 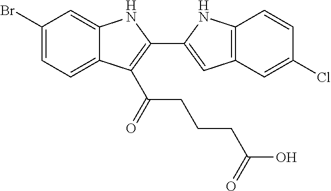 [SMILES: O=C(O)CCCC(=O)c1c(-c2cc3cc(Cl)ccc3[nH]2)[nH]c2cc(Br)ccc12] |
| 103 | 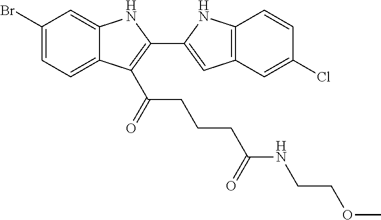 [SMILES: COCCNC(=O)CCCC(=O)c1c(-c2cc3cc(Cl)ccc3[nH]2)[nH]c2cc(Br)ccc12] |
| 104 | 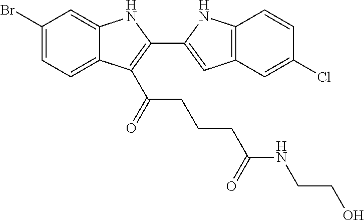 [SMILES: O=C(CCCC(=O)c1c(-c2cc3cc(Cl)ccc3[nH]2)[nH]c2cc(Br)ccc12)NCCO] |
| 105 | 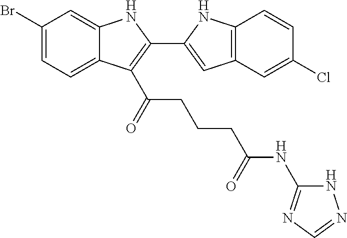 [SMILES: O=C(CCCC(=O)c1c(-c2cc3cc(Cl)ccc3[nH]2)[nH]c2cc(Br)ccc12)Nc1ncn[nH]1] |
| 106 | 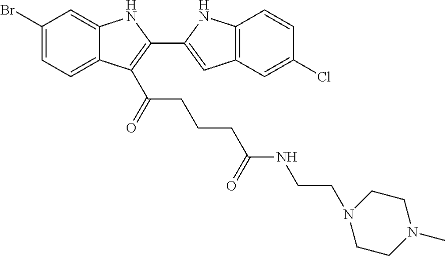 [SMILES: CN1CCN(CCNC(=O)CCCC(=O)c2c(-c3cc4cc(Cl)ccc4[nH]3)[nH]c3cc(Br)ccc23)CC1] |
| 107 | 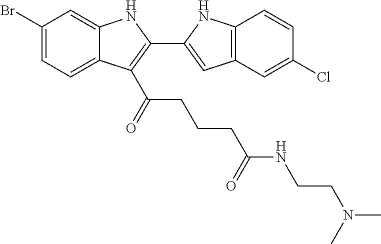 [SMILES: CN(C)CCNC(=O)CCCC(=O)c1c(-c2cc3cc(Cl)ccc3[nH]2)[nH]c2cc(Br)ccc12] |
| 108 | 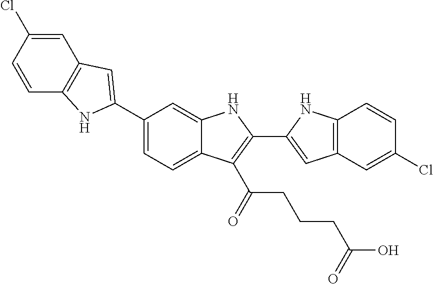 [SMILES: O=C(O)CCCC(=O)c1c(-c2cc3cc(Cl)ccc3[nH]2)[nH]c2cc(-c3cc4cc(Cl)ccc4[nH]3)ccc12] |
| 109 | 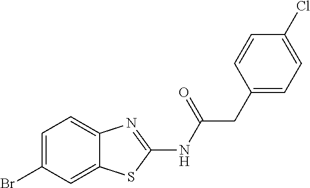 [SMILES: O=C(Cc1ccc(Cl)cc1)Nc1nc2ccc(Br)cc2s1] |
| 110 | 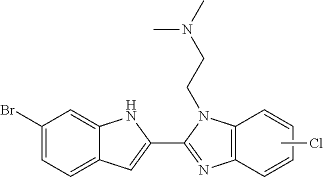 |
| 111 | 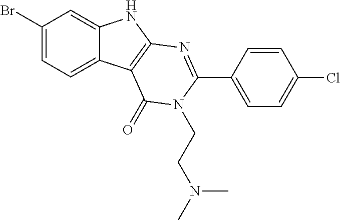 [SMILES: CN(C)CCn1c(-c2ccc(Cl)cc2)nc2[nH]c3cc(Br)ccc3c2c1=O] |
| 112 | 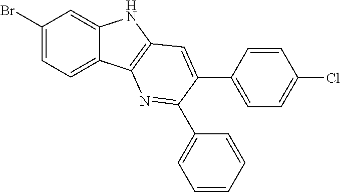 [SMILES: Clc1ccc(-c2cc3[nH]c4cc(Br)ccc4c3nc2-c2ccccc2)cc1] |
| 113 | 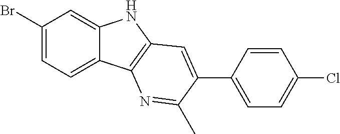 [SMILES: Cc1nc2c(cc1-c1ccc(Cl)cc1)[nH]c1cc(Br)ccc12] |
| 114 | 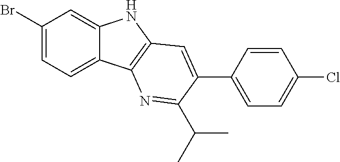 [SMILES: CC(C)c1nc2c(cc1-c1ccc(Cl)cc1)[nH]c1cc(Br)ccc12] |
| 115 | 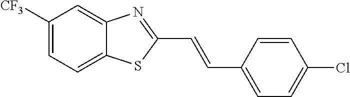 [SMILES: FC(F)(F)c1ccc2sc(/C=C/c3ccc(Cl)cc3)nc2c1] |
| 116 | 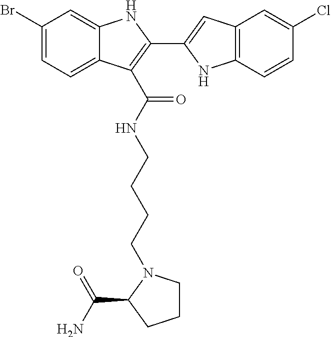 [SMILES: NC(=O)[C@@H]1CCCN1CCCCNC(=O)c1c(-c2cc3cc(Cl)ccc3[nH]2)[nH]c2cc(Br)ccc12] |
| 117 | 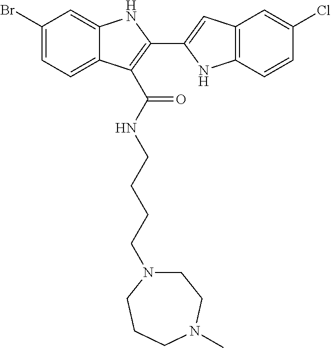 [SMILES: CN1CCCN(CCCCNC(=O)c2c(-c3cc4cc(Cl)ccc4[nH]3)[nH]c3cc(Br)ccc23)CC1] |
| 118 | 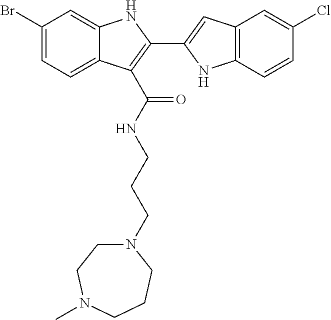 [SMILES: CN1CCCN(CCCNC(=O)c2c(-c3cc4cc(Cl)ccc4[nH]3)[nH]c3cc(Br)ccc23)CC1] |
| 119 | 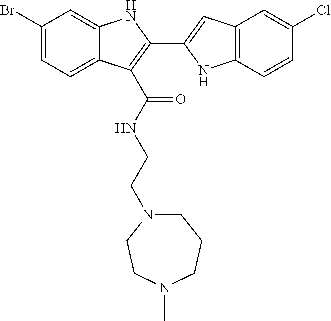 [SMILES: CN1CCCN(CCNC(=O)c2c(-c3cc4cc(Cl)ccc4[nH]3)[nH]c3cc(Br)ccc23)CC1] |
| 120 | 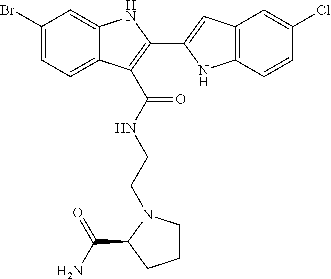 [SMILES: NC(=O)[C@@H]1CCCN1CCNC(=O)c1c(-c2cc3cc(Cl)ccc3[nH]2)[nH]c2cc(Br)ccc12] |
| 121 | 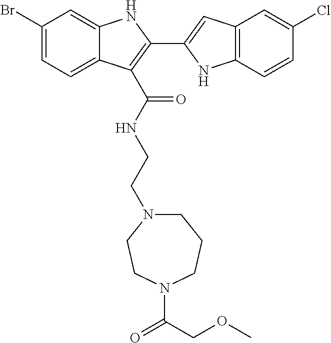 [SMILES: COCC(=O)N1CCCN(CCNC(=O)c2c(-c3cc4cc(Cl)ccc4[nH]3)[nH]c3cc(Br)ccc23)CC1] |
| 122 | 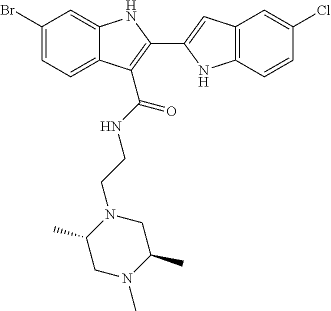 [SMILES: C[C@@H]1CN(CCNC(=O)c2c(-c3cc4cc(Cl)ccc4[nH]3)[nH]c3cc(Br)ccc23)[C@@H](C)CN1C] |
| 123 | 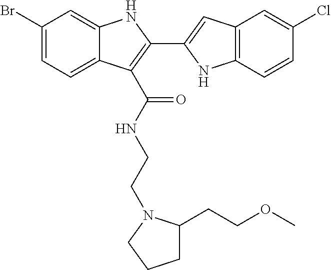 [SMILES: COCCC1CCCN1CCNC(=O)c1c(-c2cc3cc(Cl)ccc3[nH]2)[nH]c2cc(Br)ccc12] |
| 124 | 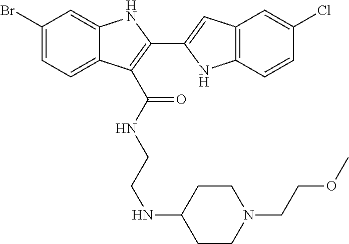 [SMILES: COCCN1CCC(NCCNC(=O)c2c(-c3cc4cc(Cl)ccc4[nH]3)[nH]c3cc(Br)ccc23)CC1] |
| 125 | 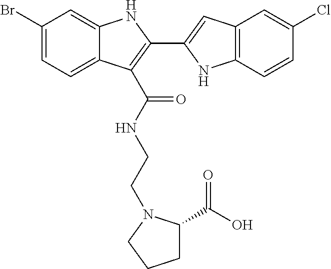 [SMILES: O=C(NCCN1CCC[C@H]1C(=O)O)c1c(-c2cc3cc(Cl)ccc3[nH]2)[nH]c2cc(Br)ccc12] |
| 126 | 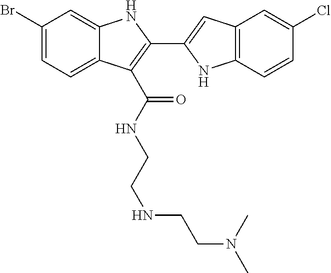 [SMILES: CN(C)CCNCCNC(=O)c1c(-c2cc3cc(Cl)ccc3[nH]2)[nH]c2cc(Br)ccc12] |
| 127 | 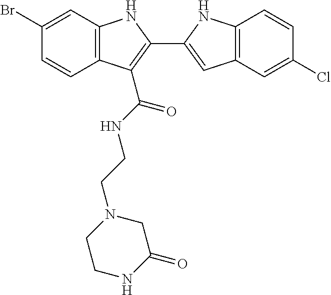 [SMILES: O=C1CN(CCNC(=O)c2c(-c3cc4cc(Cl)ccc4[nH]3)[nH]c3cc(Br)ccc23)CCN1] |
| 128 | 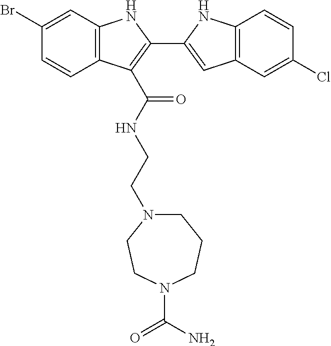 [SMILES: NC(=O)N1CCCN(CCNC(=O)c2c(-c3cc4cc(Cl)ccc4[nH]3)[nH]c3cc(Br)ccc23)CC1] |
| 129 | 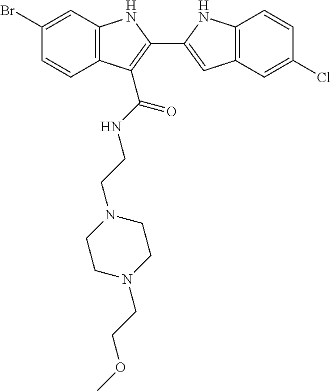 [SMILES: COCCN1CCN(CCNC(=O)c2c(-c3cc4cc(Cl)ccc4[nH]3)[nH]c3cc(Br)ccc23)CC1] |
| 130 | 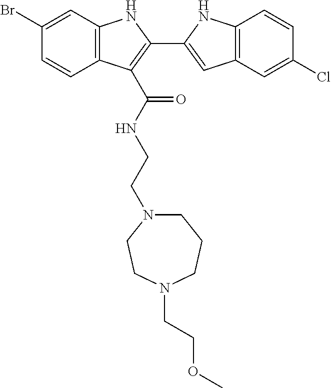 [SMILES: COCCN1CCCN(CCNC(=O)c2c(-c3cc4cc(Cl)ccc4[nH]3)[nH]c3cc(Br)ccc23)CC1] |
| 131 | 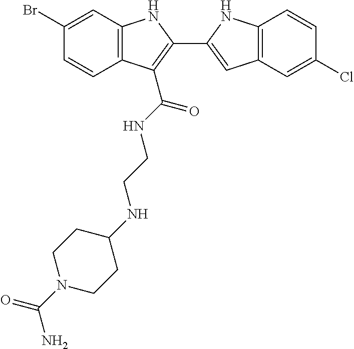 [SMILES: NC(=O)N1CCC(NCCNC(=O)c2c(-c3cc4cc(Cl)ccc4[nH]3)[nH]c3cc(Br)ccc23)CC1] |
| 132 | 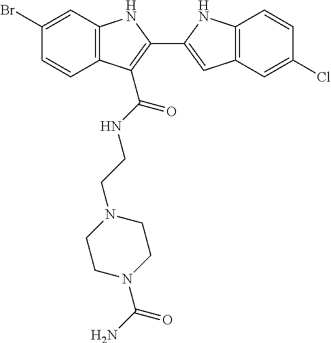 [SMILES: NC(=O)N1CCN(CCNC(=O)c2c(-c3cc4cc(Cl)ccc4[nH]3)[nH]c3cc(Br)ccc23)CC1] |
| 133 | 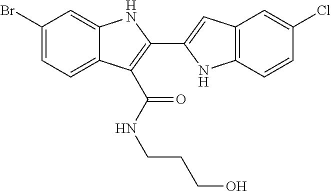 [SMILES: O=C(NCCCO)c1c(-c2cc3cc(Cl)ccc3[nH]2)[nH]c2cc(Br)ccc12] |
| 134 | 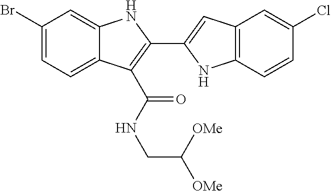 [SMILES: COC(CNC(=O)c1c(-c2cc3cc(Cl)ccc3[nH]2)[nH]c2cc(Br)ccc12)OC] |
| 135 | 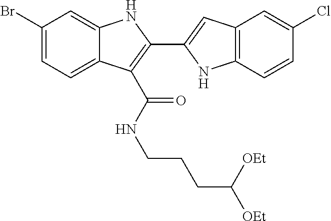 [SMILES: CCOC(CCCNC(=O)c1c(-c2cc3cc(Cl)ccc3[nH]2)[nH]c2cc(Br)ccc12)OCC] |
| 136 | 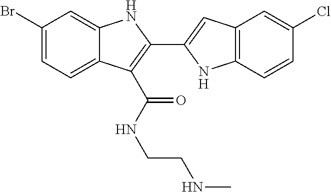 [SMILES: CNCCNC(=O)c1c(-c2cc3cc(Cl)ccc3[nH]2)[nH]c2cc(Br)ccc12] |
| 137 | 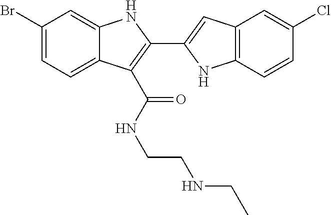 [SMILES: CCNCCNC(=O)c1c(-c2cc3cc(Cl)ccc3[nH]2)[nH]c2cc(Br)ccc12] |
| 138 | 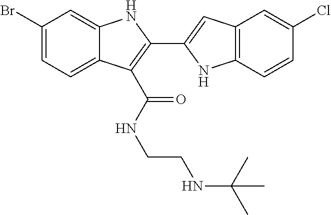 [SMILES: CC(C)(C)NCCNC(=O)c1c(-c2cc3cc(Cl)ccc3[nH]2)[nH]c2cc(Br)ccc12] |
| 139 | 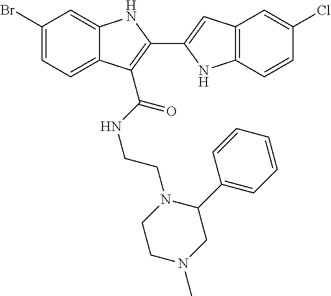 [SMILES: CN1CCN(CCNC(=O)c2c(-c3cc4cc(Cl)ccc4[nH]3)[nH]c3cc(Br)ccc23)C(c2ccccc2)C1] |
| 140 | 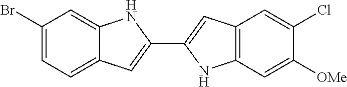 [SMILES: COc1cc2[nH]c(-c3cc4ccc(Br)cc4[nH]3)cc2cc1Cl] |
| 141 | 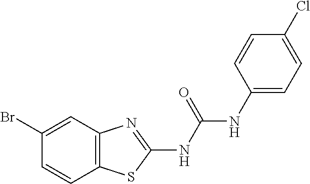 [SMILES: O=C(Nc1ccc(Cl)cc1)Nc1nc2cc(Br)ccc2s1] |
| 142 | 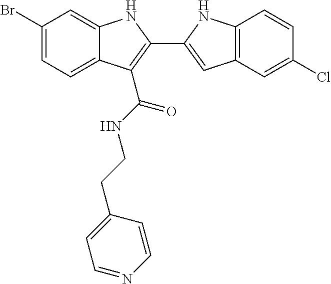 [SMILES: O=C(NCCc1ccncc1)c1c(-c2cc3cc(Cl)ccc3[nH]2)[nH]c2cc(Br)ccc12] |
| 143 | 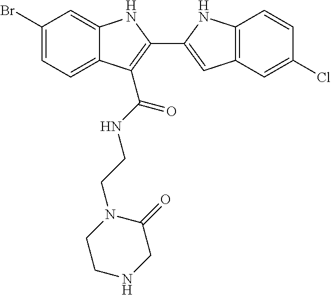 [SMILES: O=C(NCCN1CCNCC1=O)c1c(-c2cc3cc(Cl)ccc3[nH]2)[nH]c2cc(Br)ccc12] |
| 144 | 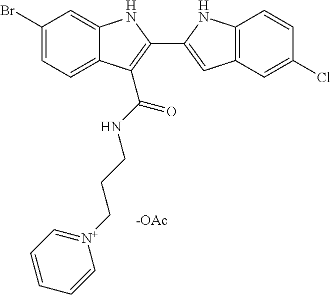 [SMILES: O=C(NCCC[n+]1ccccc1)c1c(-c2cc3cc(Cl)ccc3[nH]2)[nH]c2cc(Br)ccc12] |
| 145 | 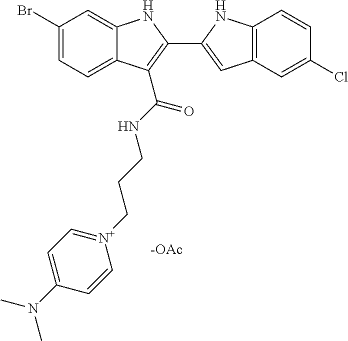 [SMILES: CN(C)c1cc[n+](CCCNC(=O)c2c(-c3cc4cc(Cl)ccc4[nH]3)[nH]c3cc(Br)ccc23)cc1] |
| 146 | 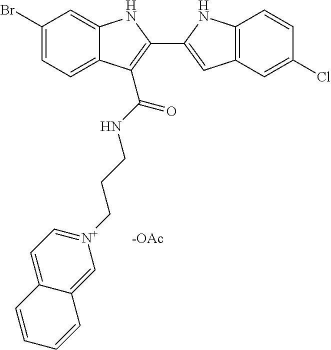 [SMILES: O=C(NCCC[n+]1ccc2ccccc2c1)c1c(-c2cc3cc(Cl)ccc3[nH]2)[nH]c2cc(Br)ccc12] |
| 147 | 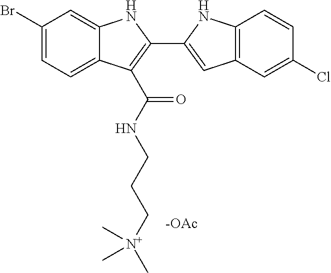 [SMILES: C[N+](C)(C)CCCNC(=O)c1c(-c2cc3cc(Cl)ccc3[nH]2)[nH]c2cc(Br)ccc12] |
| 148 | 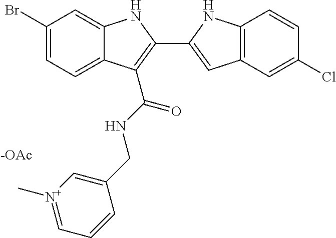 [SMILES: C[n+]1cccc(CNC(=O)c2c(-c3cc4cc(Cl)ccc4[nH]3)[nH]c3cc(Br)ccc23)c1] |
| 149 | 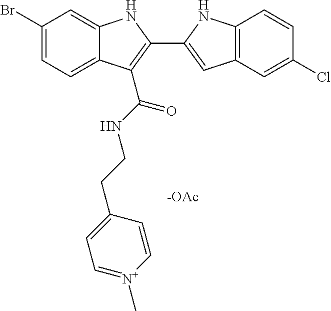 [SMILES: C[n+]1ccc(CCNC(=O)c2c(-c3cc4cc(Cl)ccc4[nH]3)[nH]c3cc(Br)ccc23)cc1] |
| 150 | 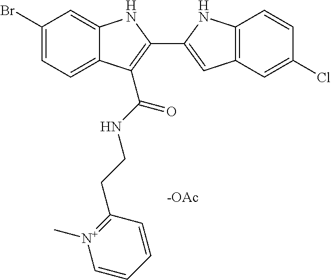 [SMILES: C[n+]1ccccc1CCNC(=O)c1c(-c2cc3cc(Cl)ccc3[nH]2)[nH]c2cc(Br)ccc12] |
| 151 | 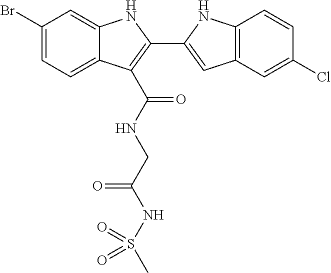 [SMILES: CS(=O)(=O)NC(=O)CNC(=O)c1c(-c2cc3cc(Cl)ccc3[nH]2)[nH]c2cc(Br)ccc12] |
| 152 | 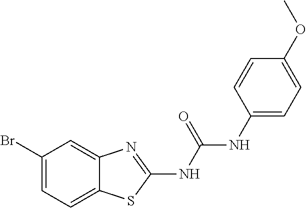 [SMILES: COc1ccc(NC(=O)Nc2nc3cc(Br)ccc3s2)cc1] |
| 153 | 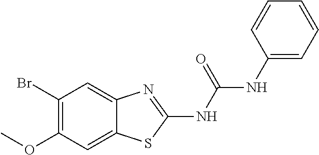 [SMILES: COc1cc2sc(NC(=O)Nc3ccccc3)nc2cc1Br] |
| 154 | 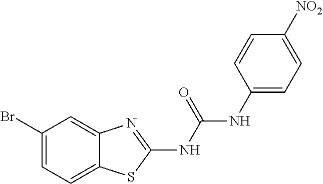 [SMILES: O=C(Nc1ccc([N+](=O)[O-])cc1)Nc1nc2cc(Br)ccc2s1] |
| 155 | 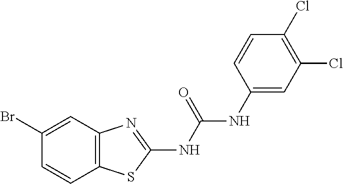 [SMILES: O=C(Nc1ccc(Cl)c(Cl)c1)Nc1nc2cc(Br)ccc2s1] |
| 156 | 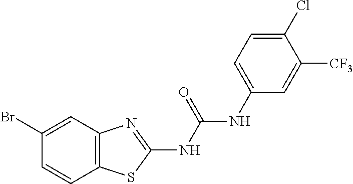 [SMILES: O=C(Nc1ccc(Cl)c(C(F)(F)F)c1)Nc1nc2cc(Br)ccc2s1] |
| 157 | 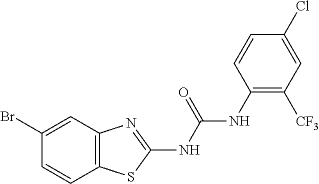 [SMILES: O=C(Nc1nc2cc(Br)ccc2s1)Nc1ccc(Cl)cc1C(F)(F)F] |
| 158 | 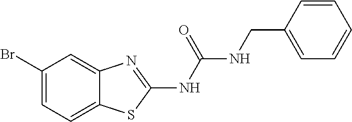 [SMILES: O=C(NCc1ccccc1)Nc1nc2cc(Br)ccc2s1] |
| 159 | 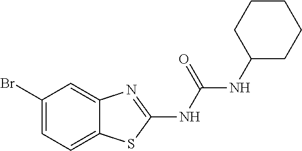 [SMILES: O=C(Nc1nc2cc(Br)ccc2s1)NC1CCCCC1] |
| 160 | 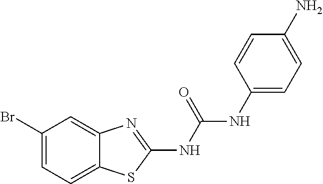 [SMILES: Nc1ccc(NC(=O)Nc2nc3cc(Br)ccc3s2)cc1] |
| 161 | 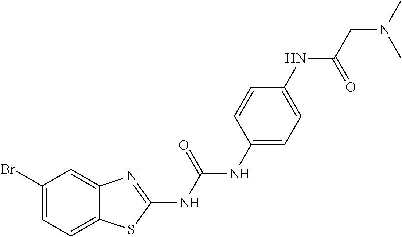 [SMILES: CN(C)CC(=O)Nc1ccc(NC(=O)Nc2nc3cc(Br)ccc3s2)cc1] |
| 162 | 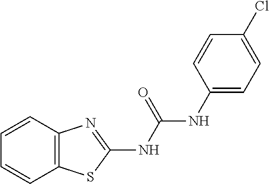 [SMILES: O=C(Nc1ccc(Cl)cc1)Nc1nc2ccccc2s1] |
| 163 | 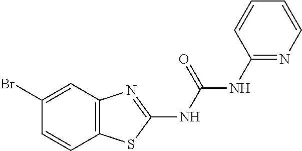 [SMILES: O=C(Nc1ccccn1)Nc1nc2cc(Br)ccc2s1] |
| 164 | 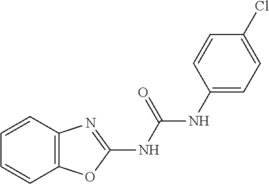 [SMILES: O=C(Nc1ccc(Cl)cc1)Nc1nc2ccccc2o1] |
| 165 | 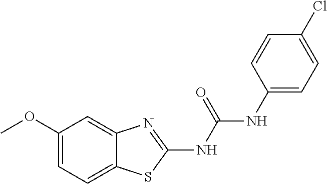 [SMILES: COc1ccc2sc(NC(=O)Nc3ccc(Cl)cc3)nc2c1] |
| 166 | 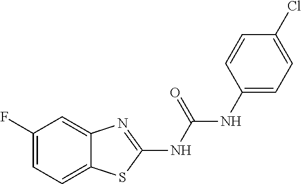 [SMILES: O=C(Nc1ccc(Cl)cc1)Nc1nc2cc(F)ccc2s1] |
| 167 | 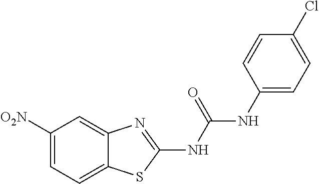 [SMILES: O=C(Nc1ccc(Cl)cc1)Nc1nc2cc([N+](=O)[O-])ccc2s1] |
| 168 | 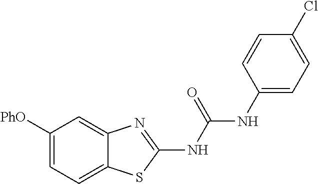 [SMILES: O=C(Nc1ccc(Cl)cc1)Nc1nc2cc(Oc3ccccc3)ccc2s1] |
| 169 | 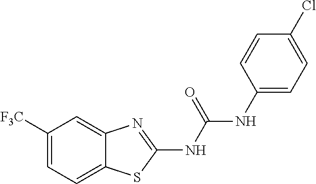 [SMILES: O=C(Nc1ccc(Cl)cc1)Nc1nc2cc(C(F)(F)F)ccc2s1] |
| 170 | 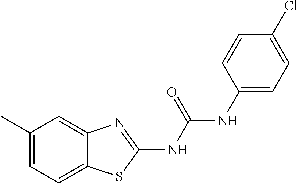 [SMILES: Cc1ccc2sc(NC(=O)Nc3ccc(Cl)cc3)nc2c1] |
| 171 | 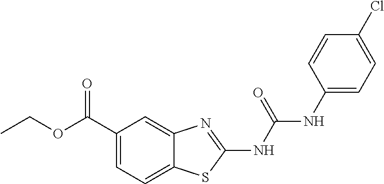 [SMILES: CCOC(=O)c1ccc2sc(NC(=O)Nc3ccc(Cl)cc3)nc2c1] |
| 172 | 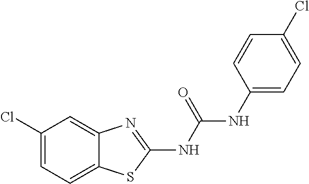 [SMILES: O=C(Nc1ccc(Cl)cc1)Nc1nc2cc(Cl)ccc2s1] |
| 173 | 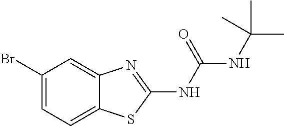 [SMILES: CC(C)(C)NC(=O)Nc1nc2cc(Br)ccc2s1] |
| 174 | 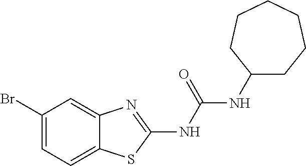 [SMILES: O=C(Nc1nc2cc(Br)ccc2s1)NC1CCCCCC1] |
| 175 | 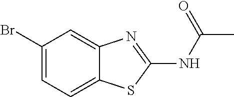 |
| 176 | 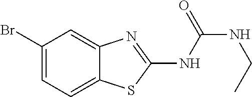 [SMILES: CCNC(=O)Nc1nc2cc(Br)ccc2s1] |
| 177 | 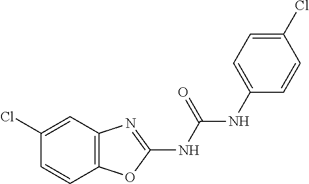 [SMILES: O=C(Nc1ccc(Cl)cc1)Nc1nc2cc(Cl)ccc2o1] |
| 178 | 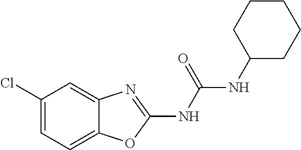 [SMILES: O=C(Nc1nc2cc(Cl)ccc2o1)NC1CCCCC1] |
| 179 | 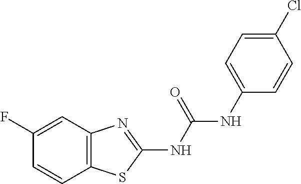 [SMILES: O=C(Nc1ccc(Cl)cc1)Nc1nc2cc(F)ccc2s1] |
| 180 | 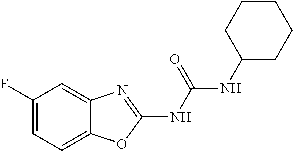 [SMILES: O=C(Nc1nc2cc(F)ccc2o1)NC1CCCCC1] |
| 181 | 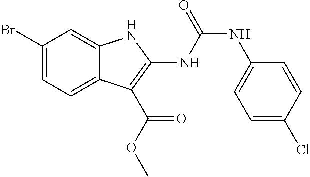 [SMILES: COC(=O)c1c(NC(=O)Nc2ccc(Cl)cc2)[nH]c2cc(Br)ccc12] |
| 182 | 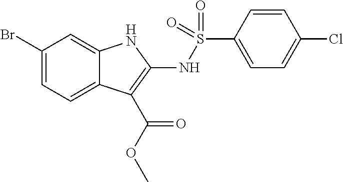 [SMILES: COC(=O)c1c(NS(=O)(=O)c2ccc(Cl)cc2)[nH]c2cc(Br)ccc12] |
| 183 | 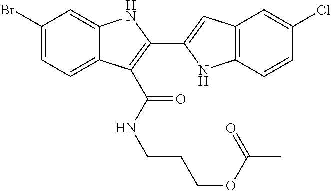 [SMILES: CC(=O)OCCCNC(=O)c1c(-c2cc3cc(Cl)ccc3[nH]2)[nH]c2cc(Br)ccc12] |
| 184 | 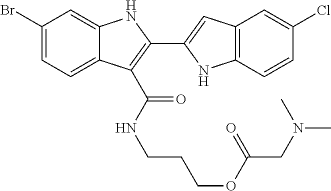 [SMILES: CN(C)CC(=O)OCCCNC(=O)c1c(-c2cc3cc(Cl)ccc3[nH]2)[nH]c2cc(Br)ccc12] |
| 185 | 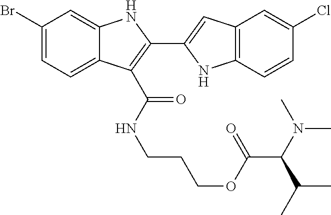 [SMILES: CC(C)[C@@H](C(=O)OCCCNC(=O)c1c(-c2cc3cc(Cl)ccc3[nH]2)[nH]c2cc(Br)ccc12)N(C)C] |
| 186 | 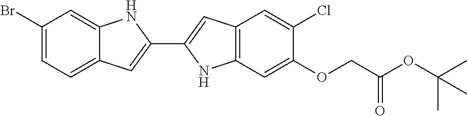 [SMILES: CC(C)(C)OC(=O)COc1cc2[nH]c(-c3cc4ccc(Br)cc4[nH]3)cc2cc1Cl] |
| 187 | 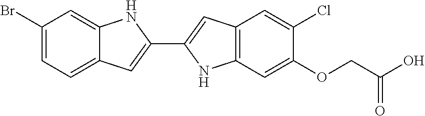 [SMILES: O=C(O)COc1cc2[nH]c(-c3cc4ccc(Br)cc4[nH]3)cc2cc1Cl] |
| 188 | 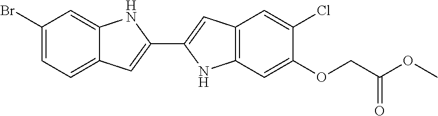 [SMILES: COC(=O)COc1cc2[nH]c(-c3cc4ccc(Br)cc4[nH]3)cc2cc1Cl] |
| 189 | 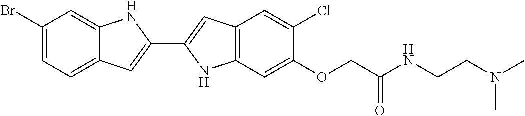 [SMILES: CN(C)CCNC(=O)COc1cc2[nH]c(-c3cc4ccc(Br)cc4[nH]3)cc2cc1Cl] |
| 190 | 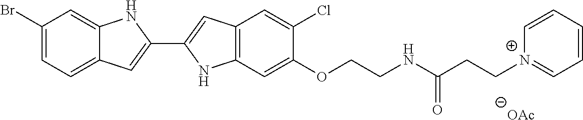 [SMILES: O=C(CC[n+]1ccccc1)NCCOc1cc2[nH]c(-c3cc4ccc(Br)cc4[nH]3)cc2cc1Cl ; "OAc"] |
| 191 | 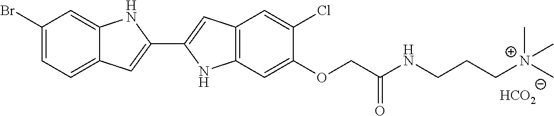 |
| 192 | 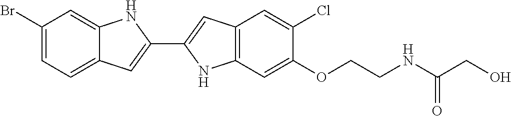 [SMILES: O=C(CO)NCCOc1cc2[nH]c(-c3cc4ccc(Br)cc4[nH]3)cc2cc1Cl] |
| 193 | 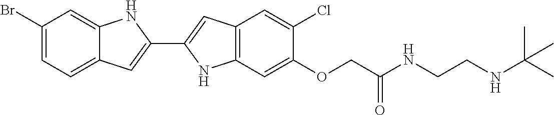 [SMILES: CC(C)(C)NCCNC(=O)COc1cc2[nH]c(-c3cc4ccc(Br)cc4[nH]3)cc2cc1Cl] |
| 194 | 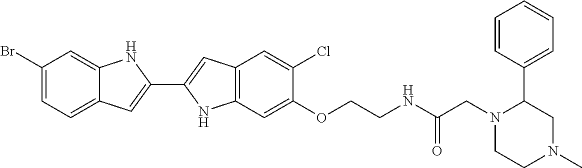 [SMILES: CN1CCN(CC(=O)NCCOc2cc3[nH]c(-c4cc5ccc(Br)cc5[nH]4)cc3cc2Cl)C(c2ccccc2)C1] |
| 195 | 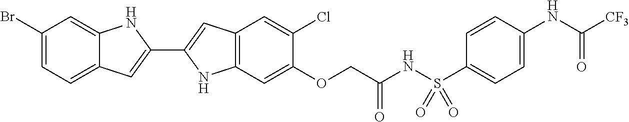 [SMILES: O=C(COc1cc2[nH]c(-c3cc4ccc(Br)cc4[nH]3)cc2cc1Cl)NS(=O)(=O)c1ccc(NC(=O)C(F)(F)F)cc1] |
| 196 | 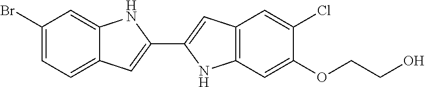 [SMILES: OCCOc1cc2[nH]c(-c3cc4ccc(Br)cc4[nH]3)cc2cc1Cl] |
| 197 | 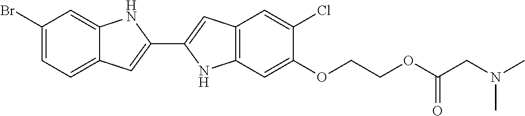 [SMILES: CN(C)CC(=O)OCCOc1cc2[nH]c(-c3cc4ccc(Br)cc4[nH]3)cc2cc1Cl] |
| 198 | 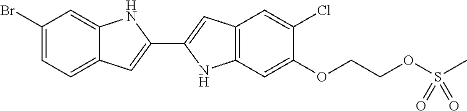 [SMILES: CS(=O)(=O)OCCOc1cc2[nH]c(-c3cc4ccc(Br)cc4[nH]3)cc2cc1Cl] |
| 199 | 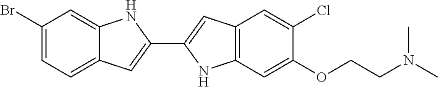 [SMILES: CN(C)CCOc1cc2[nH]c(-c3cc4ccc(Br)cc4[nH]3)cc2cc1Cl] |
| 200 | 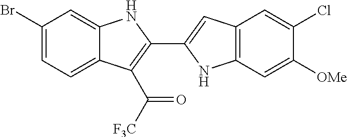 [SMILES: COc1cc2[nH]c(-c3[nH]c4cc(Br)ccc4c3C(=O)C(F)(F)F)cc2cc1Cl] |
| 201 | 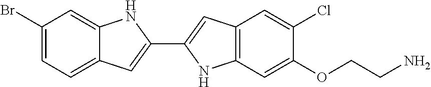 [SMILES: NCCOc1cc2[nH]c(-c3cc4ccc(Br)cc4[nH]3)cc2cc1Cl] |
| 202 | 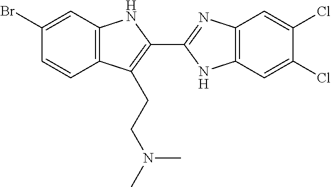 [SMILES: CN(C)CCc1c(-c2nc3cc(Cl)c(Cl)cc3[nH]2)[nH]c2cc(Br)ccc12] |
| 203 | 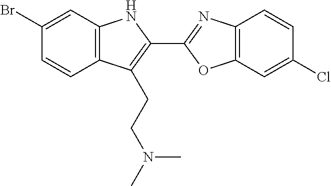 [SMILES: CN(C)CCc1c(-c2nc3ccc(Cl)cc3o2)[nH]c2cc(Br)ccc12] |
| 204 | 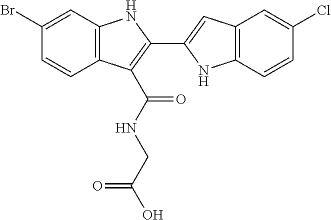 [SMILES: O=C(O)CNC(=O)c1c(-c2cc3cc(Cl)ccc3[nH]2)[nH]c2cc(Br)ccc12] |
| 205 | 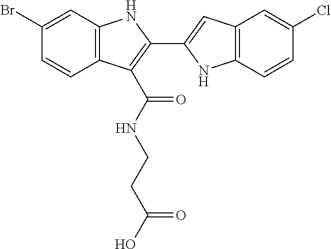 [SMILES: O=C(O)CCNC(=O)c1c(-c2cc3cc(Cl)ccc3[nH]2)[nH]c2cc(Br)ccc12] |
| 206 | 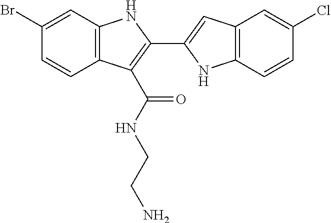 [SMILES: NCCNC(=O)c1c(-c2cc3cc(Cl)ccc3[nH]2)[nH]c2cc(Br)ccc12] |
| 207 | 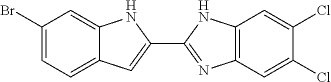 [SMILES: Clc1cc2nc(-c3cc4ccc(Br)cc4[nH]3)[nH]c2cc1Cl] |
| 208 | 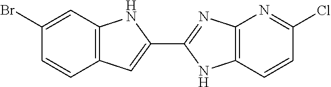 [SMILES: Clc1ccc2[nH]c(-c3cc4ccc(Br)cc4[nH]3)nc2n1] |
| 209 | 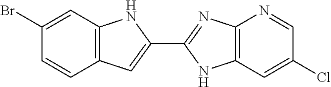 [SMILES: Clc1cnc2nc(-c3cc4ccc(Br)cc4[nH]3)[nH]c2c1] |
| 210 | 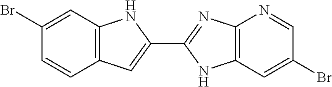 [SMILES: Brc1ccc2cc(-c3nc4ncc(Br)cc4[nH]3)[nH]c2c1] |
| 211 | 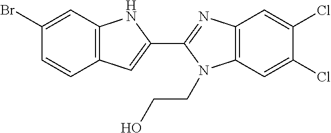 [SMILES: OCCn1c(-c2cc3ccc(Br)cc3[nH]2)nc2cc(Cl)c(Cl)cc21] |
| 212 | 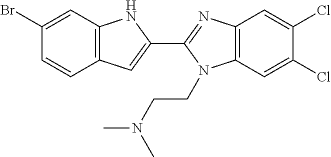 [SMILES: CN(C)CCn1c(-c2cc3ccc(Br)cc3[nH]2)nc2cc(Cl)c(Cl)cc21] |
| 213 | 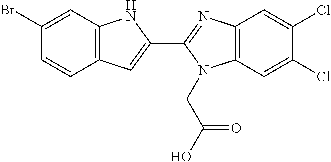 [SMILES: O=C(O)Cn1c(-c2cc3ccc(Br)cc3[nH]2)nc2cc(Cl)c(Cl)cc21] |
| 214 | 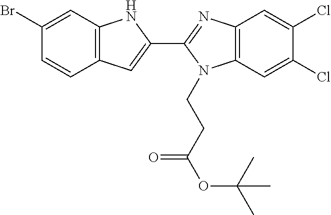 [SMILES: CC(C)(C)OC(=O)CCn1c(-c2cc3ccc(Br)cc3[nH]2)nc2cc(Cl)c(Cl)cc21] |
| 215 | 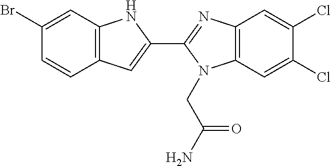 [SMILES: NC(=O)Cn1c(-c2cc3ccc(Br)cc3[nH]2)nc2cc(Cl)c(Cl)cc21] |
| 216 | 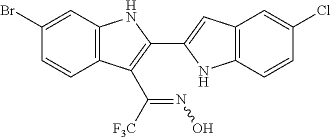 [SMILES: ON=C(c1c(-c2cc3cc(Cl)ccc3[nH]2)[nH]c2cc(Br)ccc12)C(F)(F)F] |
| 217 | 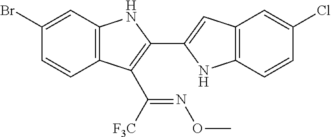 [SMILES: CO/N=C(/c1c(-c2cc3cc(Cl)ccc3[nH]2)[nH]c2cc(Br)ccc12)C(F)(F)F] |
| 218 | 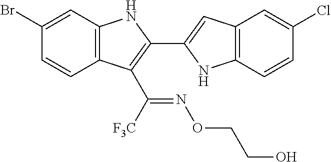 [SMILES: OCCO/N=C(/c1c(-c2cc3cc(Cl)ccc3[nH]2)[nH]c2cc(Br)ccc12)C(F)(F)F] |
| 219 | 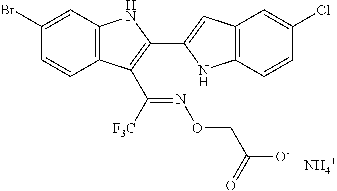 [SMILES: O=C([O-])CO/N=C(/c1c(-c2cc3cc(Cl)ccc3[nH]2)[nH]c2cc(Br)ccc12)C(F)(F)F ; [NH4+]] |
| 220 | 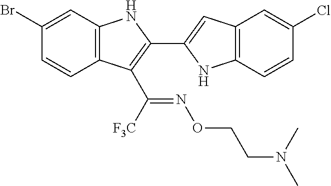 [SMILES: CN(C)CCO/N=C(/c1c(-c2cc3cc(Cl)ccc3[nH]2)[nH]c2cc(Br)ccc12)C(F)(F)F] |
| 221 | 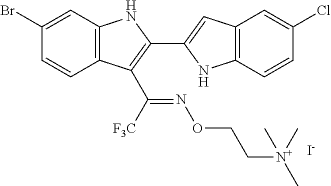 [SMILES: C[N+](C)(C)CCO/N=C(/c1c(-c2cc3cc(Cl)ccc3[nH]2)[nH]c2cc(Br)ccc12)C(F)(F)F ; [I-]] |
| 222 | 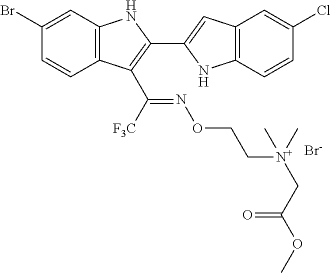 [SMILES: COC(=O)C[N+](C)(C)CCO/N=C(/c1c(-c2cc3cc(Cl)ccc3[nH]2)[nH]c2cc(Br)ccc12)C(F)(F)F ; [Br-]] |
| 223 | 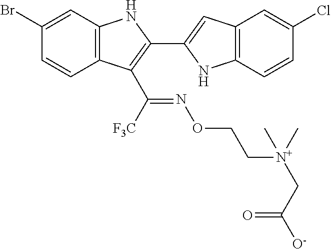 [SMILES: C[N+](C)(CCO/N=C(/c1c(-c2cc3cc(Cl)ccc3[nH]2)[nH]c2cc(Br)ccc12)C(F)(F)F)CC(=O)[O-]] |
| 224 | 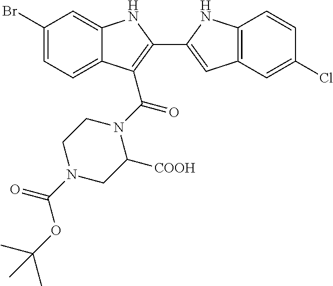 [SMILES: CC(C)(C)OC(=O)N1CCN(C(=O)c2c(-c3cc4cc(Cl)ccc4[nH]3)[nH]c3cc(Br)ccc23)C(C(=O)O)C1] |
| 225 | 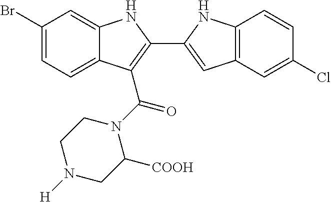 [SMILES: O=C(O)C1CNCCN1C(=O)c1c(-c2cc3cc(Cl)ccc3[nH]2)[nH]c2cc(Br)ccc12] |
| 226 | 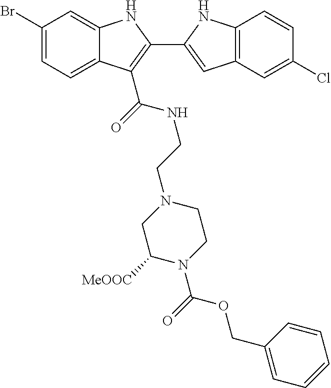 [SMILES: COC(=O)[C@@H]1CN(CCNC(=O)c2c(-c3cc4cc(Cl)ccc4[nH]3)[nH]c3cc(Br)ccc23)CCN1C(=O)OCc1ccccc1] |
| 227 | 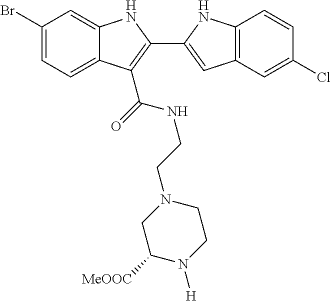 [SMILES: COC(=O)[C@@H]1CN(CCNC(=O)c2c(-c3cc4cc(Cl)ccc4[nH]3)[nH]c3cc(Br)ccc23)CCN1] |
| 228 | 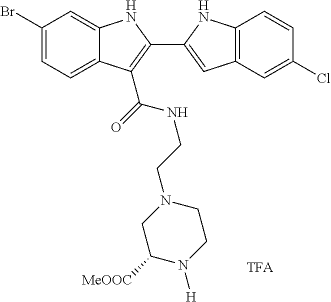 [SMILES: COC(=O)[C@@H]1CN(CCNC(=O)c2c(-c3cc4cc(Cl)ccc4[nH]3)[nH]c3cc(Br)ccc23)CCN1] |
| 229 | 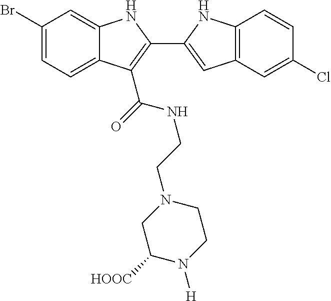 [SMILES: O=C(NCCN1CCN[C@H](C(=O)O)C1)c1c(-c2cc3cc(Cl)ccc3[nH]2)[nH]c2cc(Br)ccc12] |

In certain embodiments, the compound may be selected from: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 142, 143, 144, 145, 146, 147, 148, 149, 150, 151, 175, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, and 229, and salts thereof.

In certain embodiments, the compound may be selected from: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 102, 103, 104, 105, 106, 107, 108, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 142, 143, 144, 145, 146, 147, 148, 149, 150, 151, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, and 229, and salts thereof.

In certain embodiments, the compound may be selected from: 1, 2, 3, 4, 5, 6, 7, 8, 9, 10, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 102, 103, 104, 105, 106, 107, 108, 109, 110, 111, 112, 113, 114, 116, 117, 118, 119, 120, 121, 122, 123, 124, 125, 126, 127, 128, 129, 130, 131, 132, 133, 134, 135, 136, 137, 138, 139, 142, 143, 144, 145, 146, 147, 148, 149, 150, 151, 175, 183, 184, 185, 186, 187, 188, 189, 190, 191, 192, 193, 194, 195, 196, 197, 198, 199, 201, 202, 203, 204, 205, 206, 208, 209, 210, 212, 214, 215, 216, 217, 218, 219, 220, 221, 222, 223, 224, 225, 226, 227, 228, and 229, and salts thereof.

In certain embodiments, the compound may be selected from: 1, 2, 3, 4, 5, 6, 7, 8, 9, 10, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 102, 103, 104, 105, 106, 107, 108, 110, 111, 112, 113, 114, 116, 117, 118, 119, 120, 121, 122, 123, 124, 125, 126, 127, 128, 129, 130, 131, 132, 133, 134, 135, 136, 137, 138, 139, 142, 143, 144, 145, 146, 147, 148, 149, 150, 151, 183, 184, 185, 186, 187, 188, 189, 190, 191, 192, 193, 194, 195, 196, 197, 198, 199, 201, 202, 203, 204, 205, 206, 208, 209, 210, 212, 214, 215, 216, 217, 218, 219, 220, 221, 222, 223, 224, 225, 226, 227, 228, and 229, and salts thereof.

In certain embodiments, the compound may be selected from: 1, 2, 3, 4, 5, 6, 7, 8, 9, 10, 12, 13, 14, 16, 17, 18, 19, 21, 22, 23, 24, 25, 26, 27, 28, 30, 31, 32, 35, 36, 37, 38, 39, 40, 41, 42, 43, 44, 45, 52, 53, 54, 57, 58, 59, 60, 61, 62, 63, 64, 65, 66, 68, 69, 72, 73, 74, 77, 78, 79, 80, 81, 82, 85, 86, 87, 88, 90, 92, 93, 94, 95, 98, 102, 103, 104, 105, 106, 107, 112, 113, 114, 116, 117, 118, 119, 120, 121, 122, 123, 124, 125, 126, 127, 129, 130, 133, 136, 137, 138, 139, 142, 144, 145, 146, 147, 148, 149, 150, 183, 184, 185, 186, 187, 188, 189, 190, 191, 192, 193, 195, 196, 197, 198, 199, 201, 202, 203, 204, 205, 206, 216, 217, 218, 219, 220, 221, 222, 223, 224, and 229, and salts thereof.

In certain embodiments, the compound may be selected from: 1, 2, 3, 4, 5, 16, 18, 21, 22, 23, 27, 31, 32, 38, 39, 41, 43, 45, 52, 53, 54, 58, 60, 64, 65, 68, 69, 72, 73, 74, 77, 78, 79, 80, 81, 82, 86, 87, 92, 93, 102, 103, 104, 105, 106, 107, 113, 116, 117, 118, 119, 120, 121, 122, 123, 124, 125, 126, 127, 129, 130, 133, 136, 137, 138, 139, 142, 144, 145, 146, 147, 148, 149, 150, 183, 184, 185, 186, 187, 188, 189, 190, 191, 192, 193, 195, 196, 197, 198, 199, 201, 202, 203, 204, 205, 206, 217, 218, 219, 220, 221, 222, 224, and 229, and salts thereof.

In certain embodiments, the compound may be selected from: 1, 3, 4, 5, 16, 21, 23, 27, 31, 32, 38, 39, 58, 64, 65, 68, 69, 72, 73, 74, 78, 79, 80, 81, 82, 86, 87, 93, 102, 103, 104, 105, 106, 107, 113, 116, 117, 118, 119, 120, 121, 122, 123, 124, 125, 126, 127, 129, 130, 133, 136, 137, 138, 139, 142, 144, 145, 146, 147, 148, 149, 150, 183, 184, 185, 186, 187, 188, 189, 190, 191, 192, 193, 195, 196, 197, 198, 199, 201, 202, 203, 204, 205, 206, 217, 218, 219, 221, 224, and 229, and salts thereof.

In certain embodiments, the compound may be selected from: 3, 5, 21, 27, 38, 39, 64, 65, 68, 72, 73, 74, 79, 80, 81, 82, 102, 103, 104, 105, 106, 107, 116, 117, 118, 119, 120, 121, 122, 123, 124, 125, 126, 127, 129, 130, 133, 136, 137, 138, 139, 142, 144, 145, 147, 148, 149, 150, 183, 184, 185, 186, 187, 188, 189, 190, 192, 193, 196, 197, 198, 199, 201, 202, 203, 204, 205, 206, 217, 218, 219, and 229, and salts thereof.

In certain embodiments, the compound may be selected from: 38, 72, 79, 80, 81, 82, 102, 103, 104, 105, 106, 107, 116, 117, 118, 119, 120, 121, 122, 123, 124, 125, 126, 127, 130, 133, 136, 137, 138, 139, 142, 144, 147, 148, 149, 150, 183, 184, 185, 186, 187, 188, 190, 192, 197, 199, 201, 202, 203, 205, 206, 218, 219, and 229, and salts thereof.

In certain embodiments, the compound may be selected from: 79, 80, 102, 105, 106, 107, 116, 119, 120, 121, 122, 123, 124, 125, 126, 130, 133, 136, 142, 183, 184, 185, 186, 187, 205, and 206, and salts thereof.

In certain embodiments, the compound may be selected from: 79, 80, 102, 106, 107, 125, 133, 142, and 187, and salts thereof.

In certain embodiments, the compound may be selected from: 99, 100, 101, 141, 152, 153, 154, 155, 156, 157, 158, 159, 160, 161, 162, 163, 164, 165, 166, 167, 168, 169, 170, 171, 172, 173, 174, 176, 177, 178, 179, 180, 181, and 182, and salts thereof.

In certain embodiments, the compound may be selected from: 99, 101, 141, 152, 153, 154, 155, 156, 157, 158, 159, 160, 161, 163, 165, 166, 167, 168, 169, 170, 171, 172, 173, 174, 177, 179, 180, 181, and 182, and salts thereof.

In certain embodiments, the compound may be selected from: 99, 101, 141, 152, 155, 156, 158, 159, 160, 161, 166, 168, 169, 170, 172, 173, 174, 176, and 182, and salts thereof.

In certain embodiments, the compound may be selected from: 99, 101, 141, 152, 155, 156, 158, 159, 160, 161, 166, 168, 169, 170, 172, 173, 174, and 182, and salts thereof.

In certain embodiments, the compound may be selected from: 99, 101, 141, 152, 155, 156, 158, 159, 160, 166, 168, 169, 170, 172, 174, 176, and 182, and salts thereof.

In certain embodiments, the compound may be selected from: 99, 101, 141, 152, 155, 156, 158, 159, 160, 166, 168, 169, 170, 172, 174, and 182, and salts thereof.

In certain embodiments, the compound may be selected from: 99, 141, 152, 155, 156, 158, 159, 160, 166, 168, 169, 170, 172, and 174, and salts thereof.

In certain embodiments, the compound may be selected from: 99, 141, 152, 155, 156, 158, 159, 166, 168, 169, 172, and 174, and salts thereof.

In certain embodiments, the compound may be selected from: 99, 141, 152, 155, 156, 166, 168, 169, 172, and 174, and salts thereof.

In certain embodiments, the compound may be selected from: 141, 155, 156, and 172, and salts thereof.

In certain embodiments, the compound may be selected from: 141, 152, 155, 156, 158, 159, 160, 161, 166, 168, 169, 170, 172, 173, 174, 176, and 182, and salts thereof.

In certain embodiments, the compound may be selected from: 141, 152, 155, 156, 158, 159, 160, 161, 166, 168, 169, 170, 172, 173, 174, and 182, and salts thereof.

In certain embodiments, the compound may be selected from: 141, 152, 155, 156, 158, 159, 160, 166, 168, 169, 170, 172, 174, and 182, and salts thereof.

In certain embodiments, the compound may be selected from: 141, 152, 155, 156, 158, 159, 166, 168, 169, 170, 172, 174, and 182, and salts thereof.

In certain embodiments, the compound may be selected from: 141, 152, 155, 156, 158, 159, 166, 168, 169, 172, 174, and 182, and salts thereof.

In certain embodiments, the compound may be selected from: 141, 155, 166, 169, 172, and 182, and salts thereof.

In certain embodiments, the compound may be selected from: 152, 155, 156, 158, 160, 161, 166, 168, 169, 172, 173, 174, and 176, and salts thereof.

In certain embodiments, the compound may be selected from: 152, 155, 156, 158, 166, 169, 172, and 174, and salts thereof.

In certain embodiments, the compound may be selected from: 155, 156, 166, 169, 172, and 174, and salts thereof.

In certain embodiments, the compound may be selected from: 155, 166, 169, and 172, and salts thereof.

In certain embodiments, the compound may be selected from: 99 and 101, and salts thereof.

In certain embodiments, the compound may be selected from: 2, 3, 4, 5, 6, 7, 9, 12, 13, 14, 15, 16, 17, 18, 19, 23, 25, 26, 30, 31, 34, 40, 43, 44, 46, 52, 53, 60, 61, 65, 66, 68, 69, 72, 73, 74, 77, 93, 94, 95, 98, 102, 106, 107, 108, 112, 114, 117, 118, 119, 122, 123, 124, 126, 129, 130, 136, 137, 138, 139, 144, 145, 146, 147, 148, 149, 150, 175, 190, 191, 196, 197, 199, 201, 202, 203, 205, 206, 208, 216, 217, 218, 219, 220, 221, 222, 223, and 224, and salts thereof.

In certain embodiments, the compound may be selected from: 2, 3, 4, 5, 6, 7, 9, 12, 13, 14, 15, 16, 17, 18, 19, 23, 25, 26, 30, 31, 34, 40, 43, 44, 46, 52, 53, 60, 61, 65, 66, 68, 69, 72, 73, 74, 77, 93, 94, 95, 98, 102, 106, 107, 108, 112, 114, 117, 118, 119, 122, 123, 124, 126, 129, 130, 136, 137, 138, 139, 144, 145, 146, 147, 148, 149, 150, 190, 191, 196, 197, 199, 201, 202, 203, 205, 206, 208, 216, 217, 218, 219, 220, 221, 222, 223, and 224, and salts thereof.

In certain embodiments, the compound may be selected from: 2, 3, 4, 6, 7, 12, 13, 14, 15, 16, 17, 18, 19, 23, 25, 26, 30, 31, 34, 40, 46, 52, 53, 60, 61, 65, 66, 68, 69, 72, 73, 77, 102, 106, 107, 108, 117, 118, 119, 122, 123, 124, 126, 129, 130, 136, 137, 138, 139, 144, 145, 147, 148, 149, 150, 190, 191, 196, 197, 199, 201, 202, 203, 206, 208, 216, 217, 218, 219, 220, 221, 222, and 223, and salts thereof.

In certain embodiments, the compound may be selected from: 2, 4, 6, 7, 12, 13, 14, 15, 16, 17, 18, 19, 23, 25, 26, 40, 46, 53, 60, 61, 65, 66, 68, 72, 73, 77, 102, 106, 107, 108, 117, 122, 123, 124, 136, 137, 138, 139, 144, 145, 148, 190, 197, 199, 201, 202, 203, 206, 208, 216, 217, 218, 219, 220, 221, and 222, and salts thereof.

In certain embodiments, the compound may be selected from: 2, 12, 13, 14, 15, 16, 17, 18, 19, 46, 60, 61, 65, 66, 77, 107, 108, 138, 139, 199, 201, 202, 216, 217, 218, 220, 221, and 222, and salts thereof.

In certain embodiments, the compound may be selected from: 2, 13, 14, 15, 16, 17, 18, 19, 46, 65, 66, 108, 216, 217, 218, 220, 221, and 222, and salts thereof.

In certain embodiments, the compound may be selected from: 13, 14, 15, 16, 18, 19, 46, 65, 108, 216, 217, and 221, and salts thereof.

In certain embodiments, the compound may be selected from: 6, 7, 12, 43, 44, 61, 77, 102, 106, 107, 108, 114, 205, and 207, and salts thereof.

In certain embodiments, the compound may be selected from: 6, 7, 12, 61, 77, 102, 106, 107, 108, 114, and 207, and salts thereof.

In certain embodiments, the compound may be selected from: 12, 61, 77, 107, 108, and 207, and salts thereof.

In certain embodiments, the compound may be selected from: 117, 118, 119, 122, 123, 124, 126, 129, 130, 136, 137, 138, 139, 144, 145, 146, 147, 148, 149, 150, 175, 184, 190, 191, 196, 197, 199, 201, 202, 203, 206, 208, 216, 217, 218, 219, 220, 221, 222, 223, and 224, and salts thereof.

In certain embodiments, the compound may be selected from: 117, 118, 119, 122, 123, 124, 126, 129, 130, 136, 137, 138, 139, 144, 145, 146, 147, 148, 149, 150, 184, 190, 191, 196, 197, 199, 201, 202, 203, 206, 208, 216, 217, 218, 219, 220, 221, 222, 223, and 224, and salts thereof.

In certain embodiments, the compound may be selected from: 117, 122, 123, 124, 136, 137, 138, 139, 190, 196, 197, 199, 201, 202, 203, 206, 208, 216, 217, 218, 219, 220, 221, 222, and 223, and salts thereof.

In certain embodiments, the compound may be selected from: 199, 201, 202, 216, 217, 218, 220, 221, and 222, and salts thereof.

In certain embodiments, the compound may be selected from: 216, 217, 218, 220, 221, and 222, and salts thereof.

In certain embodiments, the compound may be selected from: 6, 7, 40, 43, 44, 61, 72, 73, 92, 102, 106, 107, 108, 114, 117, 118, 119, 122, 123, 124, 126, 129, 130, 136, 137, 138, 139, 144, 145, 147, 148, 149, 150, 175, 190, 191, 197, 199, 201, 202, 203, 206, 208, 216, 217, 218, 219, 220, 221, 222, 223, and 224, and salts thereof.

In certain embodiments, the compound may be selected from: 6, 7, 40, 43, 44, 61, 72, 73, 92, 102, 106, 107, 108, 114, 117, 118, 119, 122, 123, 124, 126, 129, 130, 136, 137, 138, 139, 144, 145, 147, 148, 149, 150, 190, 191, 197, 199, 201, 202, 203, 206, 208, 216, 217, 218, 219, 220, 221, 222, 223, and 224, and salts thereof.

In certain embodiments, the compound may be selected from: 6, 7, 40, 61, 72, 73, 106, 107, 108, 114, 117, 118, 119, 122, 123, 124, 126, 136, 137, 138, 139, 197, 199, 201, 202, 206, 216, 217, 218, 219, 220, 221, 222, and 223, and salts thereof.

In certain embodiments, the compound may be selected from: 61, 107, 108, 114, 138, 199, 201, 202, 206, 216, 217, 218, 219, 220, 221, and 222, and salts thereof.

In certain embodiments, the compound may be selected from: 108, 216, 217, 218, 220, and 221, and salts thereof.

In certain embodiments, the compound may be selected from: 6, 7, 12, 40, 43, 44, 45, 61, 72, 73, 77, 84, 102, 106, 107, 108, 114, and 207, and salts thereof.

In certain embodiments, the compound may be selected from: 6, 7, 12, 61, 72, 73, 77, 106, 107, 108, and 114, and salts thereof.

In certain embodiments, the compound may be selected from: 12, 61, 77, 107, and 108, and salts thereof.

In certain embodiments, the compound may be selected from: 12, 117, 118, 124, 126, 136, 137, 199, 201, 206, 221, and 222, and salts thereof.

In certain embodiments, the compound may be selected from: 201, 206, and 221, and salts thereof.

In certain embodiments, the compound may be selected from: 102, 199, and 201, and salts thereof.

In certain embodiments, the compound may be selected from: 2, 3, 4, 5, 6, 7, 9, 12, 13, 14, 15, 16, 17, 18, 19, 23, 25, 26, 30, 31, 34, 40, 43, 44, 46, 52, 53, 60, 61, 65, 66, 68, 69, 72, 73, 74, 77, 93, 94, 95, 98, 99, 101, 102, 106, 107, 108, 112, 114, 117, 118, 119, 122, 123, 124, 126, 129, 130, 136, 137, 138, 139, 141, 144, 145, 146, 147, 148, 149, 150, 152, 155, 156, 158, 159, 160, 161, 166, 168, 169, 170, 172, 173, 174, 175, 176, 182, 190, 191, 196, 197, 199, 201, 202, 203, 205, 206, 208, 216, 217, 218, 219, 220, 221, 222, 223, and 224, and salts thereof.

In certain embodiments, the compound may be selected from: 2, 3, 4, 5, 6, 7, 9, 12, 13, 14, 15, 16, 17, 18, 19, 23, 25, 26, 30, 31, 34, 40, 43, 44, 46, 52, 53, 60, 61, 65, 66, 68, 69, 72, 73, 74, 77, 93, 94, 95, 98, 99, 101, 102, 106, 107, 108, 112, 114, 117, 118, 119, 122, 123, 124, 126, 129, 130, 136, 137, 138, 139, 141, 144, 145, 146, 147, 148, 149, 150, 152, 155, 156, 158, 159, 160, 161, 166, 168, 169, 170, 172, 173, 174, 182, 190, 191, 196, 197, 199, 201, 202, 203, 205, 206, 208, 216, 217, 218, 219, 220, 221, 222, 223, and 224, and salts thereof.

In certain embodiments, the compound may be selected from: 2, 4, 6, 7, 12, 13, 14, 15, 16, 17, 18, 19, 23, 25, 26, 40, 46, 53, 60, 61, 65, 66, 68, 72, 73, 77, 99, 101, 102, 106, 107, 108, 117, 122, 123, 124, 136, 137, 138, 139, 141, 144, 145, 148, 152, 155, 156, 158, 159, 160, 166, 168, 169, 170, 172, 174, 176, 182, 190, 197, 199, 201, 202, 203, 206, 208, 216, 217, 218, 219, 220, 221, and 222, and salts thereof.

In certain embodiments, the compound may be selected from: 2, 4, 6, 7, 12, 13, 14, 15, 16, 17, 18, 19, 23, 25, 26, 40, 46, 53, 60, 61, 65, 66, 68, 72, 73, 77, 99, 101, 102, 106, 107, 108, 117, 122, 123, 124, 136, 137, 138, 139, 141, 144, 145, 148, 152, 155, 156, 158, 159, 160, 166, 168, 169, 170, 172, 174, 182, 190, 197, 199, 201, 202, 203, 206, 208, 216, 217, 218, 219, 220, 221, and 222, and salts thereof.

In certain embodiments, the compound may be selected from: 2, 12, 13, 14, 15, 16, 17, 18, 19, 46, 60, 61, 65, 66, 77, 99, 107, 108, 138, 139, 141, 152, 155, 156, 158, 159, 160, 166, 168, 169, 170, 172, 174, 199, 201, 202, 216, 217, 218, 220, 221, and 222, and salts thereof.

In certain embodiments, the compound may be selected from: 2, 13, 14, 15, 16, 17, 18, 19, 46, 65, 66, 99, 108, 141, 152, 155, 156, 158, 159, 166, 168, 169, 172, 174, 216, 217, 218, 220, 221, and 222, and salts thereof.

In certain embodiments, the compound may be selected from: 13, 14, 15, 16, 18, 19, 29, 46, 65, 99, 108, 141, 152, 155, 156, 166, 168, 169, 172, 174, 216, 217, and 221, and salts thereof.

In certain embodiments, the compound may be selected from: 19, 141, 155, 156, and 172, and salts thereof.

In certain embodiments, the compound may be selected from: 6, 7, 12, 43, 44, 61, 77, 102, 106, 107, 108, 114, 205, and 207, and salts thereof.

In certain embodiments, the compound may be selected from: 6, 7, 12, 61, 77, 102, 106, 107, 108, 114, and 207, and salts thereof.

In certain embodiments, the compound may be selected from: 12, 61, 77, 107, 108, and 207, and salts thereof.

In certain embodiments, the compound may be selected from: 117, 118, 119, 122, 123, 124, 126, 129, 130, 136, 137, 138, 139, 141, 144, 145, 146, 147, 148, 149, 150, 152, 155, 156, 158, 159, 160, 161, 166, 168, 169, 170, 172, 173, 174, 175, 176, 182, 184, 190, 191, 196, 197, 199, 201, 202, 203, 206, 208, 216, 217, 218, 219, 220, 221, 222, 223, and 224, and salts thereof.

In certain embodiments, the compound may be selected from: 117, 118, 119, 122, 123, 124, 126, 129, 130, 136, 137, 138, 139, 141, 144, 145, 146, 147, 148, 149, 150, 152, 155, 156, 158, 159, 160, 161, 166, 168, 169, 170, 172, 173, 174, 182, 184, 190, 191, 196, 197, 199, 201, 202, 203, 206, 208, 216, 217, 218, 219, 220, 221, 222, 223, and 224, and salts thereof.

In certain embodiments, the compound may be selected from: 117, 122, 123, 124, 136, 137, 138, 139, 141, 152, 155, 156, 158, 159, 160, 161, 166, 168, 169, 170, 172, 174, 176, 182, 190, 196, 197, 199, 201, 202, 203, 206, 208, 216, 217, 218, 219, 220, 221, 222, and 223, and salts thereof.

In certain embodiments, the compound may be selected from: 117, 122, 123, 124, 136, 137, 138, 139, 141, 152, 155, 156, 158, 159, 160, 161, 166, 168, 169, 170, 172, 174, 182, 190, 196, 197, 199, 201, 202, 203, 206, 208, 216, 217, 218, 219, 220, 221, 222, and 223, and salts thereof.

In certain embodiments, the compound may be selected from: 141, 152, 155, 156, 158, 159, 160, 166, 168, 169, 170, 172, 174, 182, 199, 201, 202, 216, 217, 218, 220, 221, and 222, and salts thereof.

In certain embodiments, the compound may be selected from: 141, 152, 155, 156, 158, 159, 166, 168, 169, 172, 174, 182, 216, and 217, and salts thereof.

In certain embodiments, the compound may be selected from: 6, 7, 40, 43, 44, 61, 72, 73, 92, 102, 106, 107, 108, 114, 117, 118, 119, 122, 123, 124, 126, 129, 130, 136, 137, 138, 139, 144, 145, 147, 148, 149, 150, 152, 155, 156, 158, 160, 161, 166, 168, 169, 172, 173, 174, 175, 176, 190, 191, 197, 199, 201, 202, 203, 206, 208, 216, 217, 218, 219, 220, 221, 222, 223, and 224, and salts thereof.

In certain embodiments, the compound may be selected from: 6, 7, 40, 43, 44, 61, 72, 73, 92, 102, 106, 107, 108, 114, 117, 118, 119, 122, 123, 124, 126, 129, 130, 136, 137, 138, 139, 144, 145, 147, 148, 149, 150, 152, 155, 156, 158, 160, 161, 166, 168, 169, 172, 173, 174, 190, 191, 197, 199, 201, 202, 203, 206, 208, 216, 217, 218, 219, 220, 221, 222, 223, and 224, and salts thereof.

In certain embodiments, the compound may be selected from: 6, 7, 40, 61, 72, 73, 106, 107, 108, 114, 117, 118, 119, 122, 123, 124, 126, 136, 137, 138, 139, 152, 155, 156, 158, 160, 166, 169, 172, 174, 197, 199, 201, 202, 206, 216, 217, 218, 219, 220, 221, 222, and 223, and salts thereof.

In certain embodiments, the compound may be selected from: 61, 108, 138, 152, 155, 156, 158, 166, 169, 172, 174, 216, 217, 218, 220, and 221, and salts thereof.

In certain embodiments, the compound may be selected from: 6, 7, 12, 40, 43, 44, 45, 61, 72, 73, 77, 84, 99, 101, 102, 106, 107, 108, and 114, and salts thereof.

In certain embodiments, the compound may be selected from: 12, 61, 77, 107, and 108, and salts thereof.

In certain embodiments, the compound may be selected from: 12, 117, 118, 124, 126, 136, 137, 199, 201, 206, 221, and 222, and salts thereof.

In certain embodiments, the compound may be selected from: 201, 206, and 221, and salts thereof.

In certain embodiments, the compound may be selected from: 102, 199, and 201, and salts thereof.

Methods of making compounds of the present invention are provided herein and specifically in the Examples section of the application.

Compounds of the present invention may be used and are useful in the treatment, prophylactic or otherwise, of bacterial infection. In some embodiments, the bacterial infection may be caused by a Gram positive or a Gram negative bacterial species. In some embodiments, the bacterial species is selected from Enterobacteriales, Bacteriodales, Legionellales, Neisseriales, Pseudomonales, Vibrionales, Pasterrellales and Camylobacterales. In some embodiments, the bacterial species is selected from Acinetobacter, Actinobacillus, Bordetella, Brucella, Bartonella, Campylobacter, Cyanobacteria, Enterobacter, Erwinia, Escherichia coli, Franciscella, Helicobacter, Hemophilus, Klebsiella, Legionella, Moraxella, Neisseria, Pasteurella, Proteus, Pseudomonas, Salmonella, Serratia, Shigella, Treponema, Vibrio and Yersinia. In some embodiments, the bacterial species is selected from E. coli, Klebsiella pneumoniae, Acinetobacter baumannii, Pseudomonas aeruginosa, Neisseria gonorrhoeae, Salmonella typhimurium and Neisseria meningitis. In some embodiments, the bacterial species is selected from Staphylococcus, Streptococcus, Enterococcus (including Vancomycin-resistant Enterococcus faecalis: VRE), Bacillus and Listeria. In some embodiments, the bacterial species is selected from Staphyloccus saprophytics, Staphyloccocus xylosus, Staphyloccocus lugdunensis, Staphyloccocus schleiferi, Stapylococcus caprae, Staphylococcus epidermidis, Staphylococcus hominis, Staphylococcus saprophytics, Staphylococcus warneri, Staphylococcus aureus, MRSA, Enterococcus faecalis, Enterococcus faecium (including Vancomycin-resistant enterococcus VRE), Proprionibacterium acnes, Bacillus cereus, Bacillus subtilis, Listeria monocytogenes, Streptococcus pyogenes, Streptococcus salivarius, Streptococcus mutans and Streptococcus pneumoniae.

The antimicrobial activity of compounds of the present invention against S. aureus ATCC 29123 (or another bacterial species of interest) may be tested in vivo using a thigh infection model in neutropenic mice. Briefly, animals (e.g. female CD-1 mice, 5 weeks of age) may be made neutropenic prior to S. aureus (or other bacterial species of interest) thigh infection by pre-treating with cyclophosphamide (e.g. 150 mg/kg, IP, −4 and −1 days pre-inoculation). On the inoculation day (day 0), mice can be infected with S. aureus (or other bacterial species of interest) at time zero (t=0). Animals are then individually monitored for adverse reactions for 30 min post-infection.

A compound of the present of invention for testing may be prepared for IV administration by dissolving in 3% DMSO/6% Solutol® HS 15/10 mM PB (pH7.4) and/or for oral administration by dissolving in 3% DMSO/6% Solutol® HS 15/water. Vancomycin may also be administered as a solution in PBS. The test compounds may be administered at 2 and 8 hours post-infection and animals individually monitored for adverse reactions for 30 min after each injection. All animals are monitored hourly from 20 hours post infection to an endpoint (e.g. t=24 hr post infection). At a determined timepoint, animals are sacrificed and the injected thighs collected.

Quantitative enumeration of bacterial load may then be determined by plating serial dilutions from homogenized thigh muscles. Homogenized muscle could, for example, be in a total of 2 mL volume, from which a 1 in 10 dilution may be prepared (100 μL into 900 μL saline). From this a series of dilutions may be prepared and plated on Mueller Hinton agar plates. Plates are incubated for a period of time, (e.g. overnight) at suitable conditions (e.g. 37° C. in 100% atmospheric air). At the end of the period of time colony counts may be determined and the final CFU per mL calculated.

Compounds of the present invention may be formulated into a pharmaceutical formulation. Many compounds of this invention or for use in this invention are generally water soluble and may be formed as salts. In such cases, pharmaceutical compositions in accordance with this invention may comprise a salt of such a compound, preferably a physiologically acceptable salt, which are known in the art. Pharmaceutical preparations will typically comprise one or more carriers acceptable for the mode of administration of the preparation, be it by injection, inhalation, topical administration, lavage, or other modes suitable for the selected treatment. Suitable carriers are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20^{th} ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Compounds or pharmaceutical compositions in accordance with this invention or for use in this invention may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

An “effective amount” of a pharmaceutical composition according to the invention includes a therapeutically effective amount or a prophylactically effective amount. A “therapeutically effective amount” refers to an amount effective, at dosages and for periods of time necessary, to achieve the desired therapeutic result, such as a reduction in a bacterial population in a subject. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as prevention of a bacterial infection or reduced ill effects from bacterial activity in a subject. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

As used herein, a “subject” may be a human, non-human primate, rat, mouse, cow, horse, pig, sheep, goat, dog, cat, etc. The subject may be suspected of having or at risk for having a bacterial infection. Diagnostic methods for various bacterial infections and the clinical delineation of bacterial infections diagnoses are known to those of ordinary skill in the art.

Illustrative embodiments of the present invention provide a pharmaceutical formulation comprising a compound described herein and a pharmaceutically acceptable excipient.

Illustrative embodiments of the present invention provide use of a compound described herein in the preparation of a medicament for treatment or prophylactic treatment of bacterial infection.

Illustrative embodiments of the present invention provide use of a compound described herein for treatment or prophylactic treatment of bacterial infection.

Illustrative embodiments of the present invention provide a method of treatment comprising administering an effective amount of a compound described herein to a subject having, suspected of having or at risk for having bacterial infection.

Compounds described herein may also be used for non-medicinal purposes. Such non-medicinal purposes are generally related to introducing or applying a compound described here to a surface in order to reduce or inhibit the prevalence of bacteria on the surface. The reduction or inhibiting the prevalence may be prophylactic or otherwise. Such non-medicinal uses include, but are not limited to cleaning surfaces, hand washing, plant protection to control various bacterial and fungal diseases, food preservation, and as an adjunct in a microbiological technique, for example in a tissue culture.

### EXAMPLES

The following examples are illustrative of some of the embodiments of the invention described herein. These examples do not limit the spirit or scope of the invention in any way.

### Examples

### General Methods and Equipment

¹H and ¹³C NMR spectra were recorded with either Bruker Avance II™ 600 MHz, Bruker Avance III™ 500 MHz, Bruker Avance III™ 400 MHz or Bruker Avance II⁺. Processing of the spectra was performed with MestRec™ software. Mass spectra were recorded using a Waters Micromass ZQ mass spectrometer. Analytical thin-layer chromatography (TLC) was performed on aluminum plates pre-coated with silica gel 60F-254 as the absorbent. The developed plates were air-dried, exposed to UV light and/or dipped in KMnO₄ solution and heated. Silica gel chromatography was carried out on Biotage Isolera Flash Purification Systems using commercial 50 μm silica gel cartridges. Purity (>90%) for all final compounds was confirmed by analytical reverse-phase HPLC utilizing either a Dikma Technologies™ Inspire® C18 reverse-phase analytical column (4.6×150 mm) or Waters Symmetry C18 reverse-phase analytical column (4.6×75 mm). All HPLC purifications were carried out using an Agilent™ C18 reverse-phase preparatory column (21.2×250 mm).

### Synthesis of Intermediate 1-iii

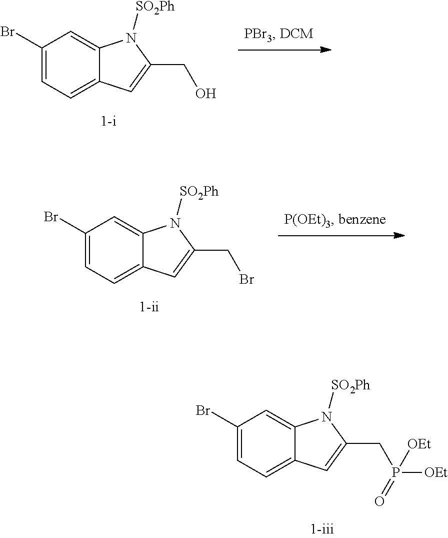

### Intermediate 1-iii: Diethyl ((6-bromo-1-(phenylsulfonyl)-1H-indol-2-yl)methyl)phosphonate

To a stirred solution of 1-i (300 mg, 0.82 mmol) (Kumar N. S et al. Bioorg. Med. Chem. 22 (2014) 1708-1725)) in DCM (5 mL) at 0° C. under Ar was added PBr₃ (90 μL, 0.96 mmol) and the mixture was stirred at rt for 1 h. The mixture was re-cooled to 0° C. and then quenched with saturated NaHCO₃ (5 mL). The mixture was partitioned between EtOAc and H₂O and the organic layer was washed with brine, dried over anhydrous Na₂SO₄, filtered, and concentrated under reduced pressure to provide crude intermediate 1-ii. 1-ii was dissolved in benzene (1 mL) and triethyl phosphite (1 mL), and the resulting mixture was refluxed for 16 h. Volatiles were removed by distillation and the residue was purified by silica gel chromatography, eluting with MeOH/DCM, to provide intermediate 1-iii as brown solid (295 mg, 74%).

### General Method I

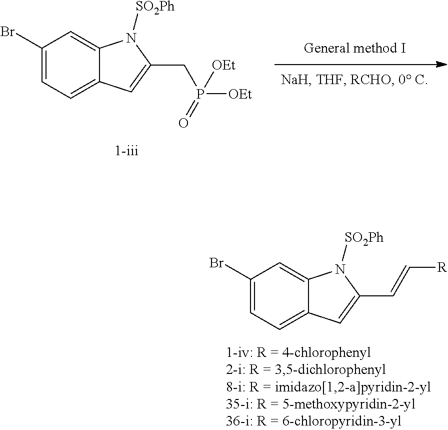

### Intermediates 1-iv, 2-i, 8-i, 35-i or 36-i

To a stirred solution of 1-iii (1 mmol) in THF (7 mL) at 0° C. was added NaH (60% in oil, 1.5 mmol) and the mixture was stirred for 20 min followed by the addition of the corresponding aldehyde (2.0 mmol) in THF (2 mL). The mixture was stirred at 0° C. for 2 h and then partitioned between EtOAc and H₂O. The organic layer was washed with brine, dried over anhydrous Na₂SO₄, filtered and then concentrated under reduced pressure. The residue was partially purified by silica gel chromatography, eluting with an either EtOAc/hexanes or MeOH/DCM gradient, to provide the corresponding alkene intermediate.

### General Method II and III

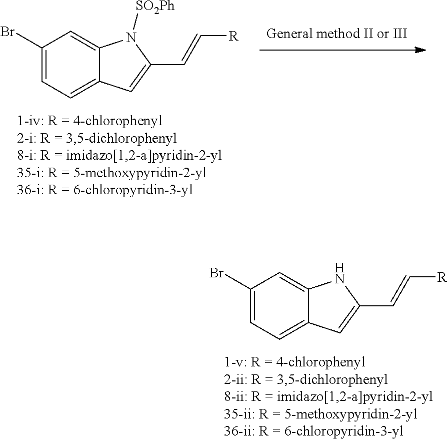

General Method II: Cs₂CO₃, MeOH-THF, 90° C.

General Method III: TBAF, THF, rt

### General Method II

Intermediate 1-iv, 2-i, 8-i or 35-i was dissolved in THF (6 mL) and MeOH (12 mL). Cs₂CO₃ (2 mmol) was added and the mixture was heated with microwave at 90° C. for 30 min. The mixture was diluted with EtOAc and H₂O. The organic layer was separated, washed with brine, dried over anhydrous Na₂SO₄, filtered and then concentrated under reduced pressure. The crude product was purified by silica gel chromatography, eluting with an EtOAc/hexanes or MeOH/DCM gradient, to provide the desired indole intermediates.

### General Method III

Intermediate 36-i was dissolved in THF (5 mmol) followed by the addition of TBAF (1M in THF, 2 mmol). The mixture was stirred at rt for 16 h and then diluted with EtOAc and H₂O The organic layer was separated, washed with brine, dried over anhydrous Na₂SO₄, filtered and then concentrated under reduced pressure. The crude product was purified by silica gel chromatography, eluting with an EtOAc/hexanes gradient, to provide the desired indole intermediates.

### Intermediate 1-v: (E)-6-Bromo-2-(4-chlorostyryl)-1H-indole

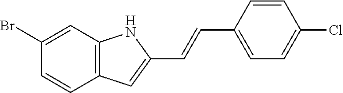 [SMILES: Clc1ccc(/C=C/c2cc3ccc(Br)cc3[nH]2)cc1]

Prepared according to general method I and II from 1-iii and 4-chlorobenzaldehyde (550 mg, 50%). ¹H NMR (500 MHz, CDCl₃) δ 8.23 (s, 1H), 7.56-7.41 (m, 4H), 7.36 (d, J=8.0 Hz, 2H), 7.23 (d, J=8.0 Hz, 1H), 7.08 (d, J=16.4 Hz, 1H), 6.88 (d, J=16.2 Hz, 1H), 6.61 (s, 1H). HRMS calc for (C₁₆H₁₁BrClN−H)⁻ 331.9669, found 331.9665.

### Intermediate 2-ii: (E)-6-Bromo-2-(3,5-dichlorostyryl)-1H-indole

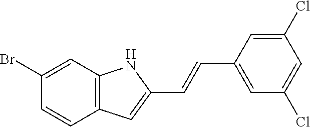 [SMILES: Clc1cc(Cl)cc(/C=C/c2cc3ccc(Br)cc3[nH]2)c1]

Prepared according to general method I and II from 1-iii and 3,5-dichlorobenzaldehyde (65 mg, 37%). ¹H NMR (400 MHz, DMSO) δ 11.57 (s, 1H), 7.63 (d, J=1.8 Hz, 2H), 7.54-7.49 (m, 3H), 7.47 (d, J=11.5 Hz, 1H), 7.15-7.08 (m, 2H), 6.66 (s, 1H). Mass calculated for (C₁₆H₁₀BrCl₂N+H)⁺365.9, found 365.8.

### Intermediate 8-ii: (E)-2-(2-(6-Bromo-1H-indol-2-yl)vinyl)imidazo[1,2-a]pyridine

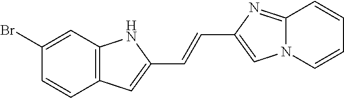 [SMILES: Brc1ccc2cc(/C=C/c3cn4ccccc4n3)[nH]c2c1]

Prepared according to general method I and II from 1-iii and imidazo[1,2-a]pyridine-2-carbaldehyde (105 mg, 76%). ¹H NMR (400 MHz, DMSO) δ 11.56 (s, 1H), 8.50 (d, J=6.8 Hz, 1H), 8.08 (s, 1H), 7.55-7.48 (m, 2H), 7.46 (d, J=8.4 Hz, 1H), 7.42 (d, J=16.1 Hz, 1H), 7.29-7.21 (m, 2H), 7.11 (dd, J=8.4, 1.8 Hz, 1H), 6.88 (td, J=6.7, 1.1 Hz, 1H), 6.64 (d, J=1.3 Hz, 1H). Mass calculated for (C₁₇H₁₂BrN₃+H)⁺338.0, found 338.1.

### Intermediate 35-ii: (E)-6-Bromo-2-(2-(5-methoxypyridin-2-yl)vinyl)-1H-indole

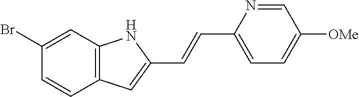 [SMILES: COc1ccc(/C=C/c2cc3ccc(Br)cc3[nH]2)nc1]

Prepared according to general method I and II from 1-iii and 5-methoxypicolinaldehyde (85 mg, 63%). ¹H NMR (400 MHz, DMSO) δ 11.58 (s, 1H), 8.32 (d, J=2.9 Hz, 1H), 7.56-7.44 (m, 4H), 7.41 (dd, J=8.7, 3.0 Hz, 1H), 7.23 (d, J=16.2 Hz, 1H), 7.11 (dd, J=8.4, 1.8 Hz, 1H), 6.66 (d, J=1.3 Hz, 1H), 3.87 (s, 3H). Mass calculated for (C₁₆H₁₃BrN₂O+H)⁺329.0, found 329.1.

### Intermediate 36-ii: (E)-6-Bromo-2-(2-(6-chloropyridin-3-yl)vinyl)-1H-indole

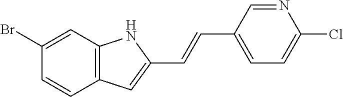 [SMILES: Clc1ccc(/C=C/c2cc3ccc(Br)cc3[nH]2)cn1]

Prepared according to general method I and III from 1-iii and 6-chloronicotinaldehyde (145 mg, 60%). ¹H NMR (400 MHz, DMSO) δ 11.63 (s, 1H), 8.56 (d, J=2.5 Hz, 1H), 8.12 (dd, J=8.5, 2.5 Hz, 1H), 7.57-7.47 (m, 3H), 7.44 (d, J=16.5 Hz, 1H), 7.19 (d, J=16.6 Hz, 1H), 7.13 (dd, J=8.4, 1.8 Hz, 1H), 6.66 (s, 1H). Mass calculated for (C₁₅H₁₀BrClN₂+H)⁺335.0, found 334.9.

### General Method IV

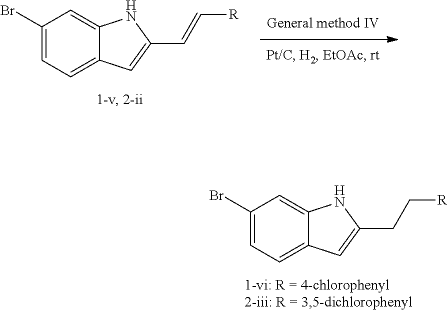

To a stirred solution of the appropriate indole (1 mmol) in EtOAc (25 mL) was added Pt/C (10% dry on C, 50 mg) and the mixture was stirred under H₂ (1 atm) for 16 h. The resulting mixture was filtered through a pad of celite and concentrated under reduced pressure. The crude product was purified by silica gel chromatography, eluting with an EtOAc/hexanes gradient, to provide the desired product.

### Intermediate 1-vi: 6-Bromo-2-(4-chlorophenethyl)-1H-indole

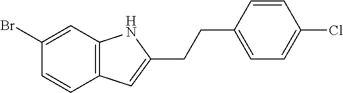 [SMILES: Clc1ccc(CCc2cc3ccc(Br)cc3[nH]2)cc1]

Prepared according to general method IV from intermediate 1-v (240 mg, 90%). ¹H NMR (500 MHz, CDCl₃) δ 7.75 (s, 1H), 7.44 (d, J=1.4 Hz, 1H), 7.40 (d, J=8.4 Hz, 1H), 7.29 (d, J=8.3 Hz, 2H), 7.20 (dd, J=8.4, 1.7 Hz, 1H), 7.13 (d, J=8.3 Hz, 2H), 6.24 (s, 1H), 3.10-2.97 (m, 4H). HRMS calc for (C₁₆H₁₃BrClN−H)⁻ 333.9825, found 333.9825.

### Intermediate 2-iii: 6-Bromo-2-(3,5-dichlorophenethyl)-1H-indole

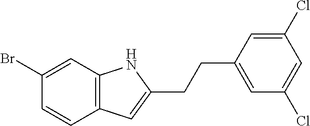 [SMILES: Clc1cc(Cl)cc(CCc2cc3ccc(Br)cc3[nH]2)c1]

Prepared according to general method IV from intermediate 2-ii (70 mg, 88%). ¹H NMR (400 MHz, DMSO) δ 11.13 (s, 1H), 7.47 (s, 1H), 7.42 (t, J=1.9 Hz, 1H), 7.37 (d, J=8.4 Hz, 1H), 7.35 (d, J=1.9 Hz, 2H), 7.05 (dd, J=8.4, 1.8 Hz, 1H), 6.18 (d, J=1.4 Hz, 1H), 3.03 (s, 4H). Mass calculated for (C₁₆H₁₂BrCl₂N−H)⁻ 366.0, found 365.9.

### General Method V

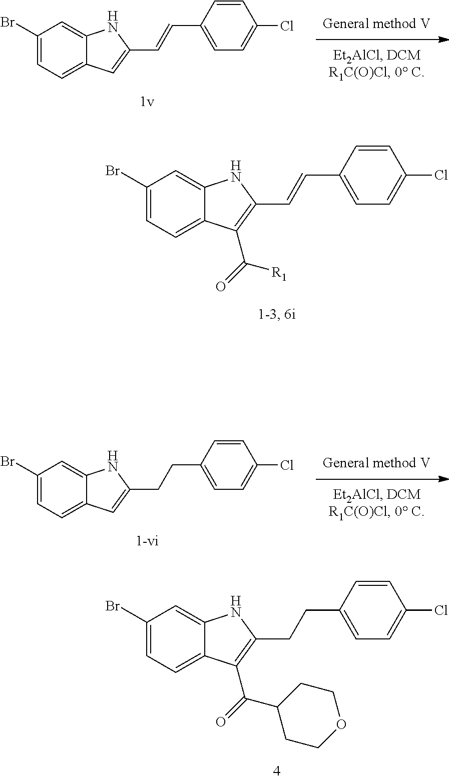

To a stirred solution of the corresponding indole (0.1 mmol) in DCM (1.6 mL) under Ar at 0° C. was added Et₂AlCl (1M in hexanes, 0.45 mmol) and the mixture was stirred at 0° C. for 30 min. The corresponding acid chloride (0.45 mmol) in DCM (1 mL) was added and the mixture was stirred at 0° C. for 3 h, quenched with saturated aqueous NaHCO₃ and then partitioned between EtOAc and H₂O. The organic layer was washed with brine, dried over anhydrous Na₂SO₄, filtered and concentrated under reduced pressure. The crude product was purified by silica gel chromatography, eluting with an EtOAc/hexanes gradient, to provide the corresponding 3-acylindole.

### Compound 1: (E)-(6-Bromo-2-(4-chlorostyryl)-1H-indol-3-yl)(tetrahydro-2H-pyran-4-yl)methanone

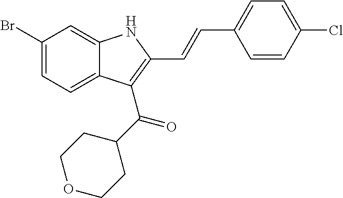 [SMILES: O=C(c1c(/C=C/c2ccc(Cl)cc2)[nH]c2cc(Br)ccc12)C1CCOCC1]

Prepared according to general method V from intermediate 1-v and tetrahydro-2H-pyran-4-carbonyl chloride (13 mg, 19%). ¹H NMR (600 MHz, DMSO) δ 12.39 (s, 1H), 7.95 (d, J=16.7 Hz, 1H), 7.89 (d, J=8.6 Hz, 1H), 7.66 (d, J=8.4 Hz, 2H), 7.61 (d, J=1.7 Hz, 1H), 7.53 (d, J=8.5 Hz, 2H), 7.46 (d, J=16.6 Hz, 1H), 7.35 (dd, J=8.6, 1.8 Hz, 1H), 3.92 (d, J=10.4 Hz, 2H), 3.57-3.47 (m, 3H), 1.78 (d, J=11.6 Hz, 2H), 1.72-1.63 (m, 2H). Mass calculated for (C₂₂H₁₉BrClNO₂−H)⁻ 444.0, found 444.0.

### Compound 2: (E)-(6-Bromo-2-(4-chlorostyryl)-1H-indol-3-yl)(cyclopropyl)methanone

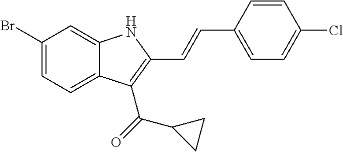 [SMILES: O=C(c1c(/C=C/c2ccc(Cl)cc2)[nH]c2cc(Br)ccc12)C1CC1]

Prepared according to general method V from intermediate 1-v and cyclopropanecarbonyl chloride (48 mg, 53%). ¹H NMR (600 MHz, DMSO) δ 12.33 (s, 1H), 7.97 (d, J=8.5 Hz, 1H), 7.86 (d, J=16.7 Hz, 1H), 7.65-7.60 (m, 3H), 7.51 (d, J=8.0 Hz, 2H), 7.46 (d, J=16.7 Hz, 1H), 7.34-7.31 (m, 1H), 2.67-2.62 (m, 1H), 1.14-1.06 (m, 4H). Mass calculated for (C₂₀H₁₅BrClNO−H)⁻ 400.0, found 400.0.

### Compound 3: (E)-(6-Bromo-2-(4-chlorostyryl)-1H-indol-3-yl)(phenyl)methanone

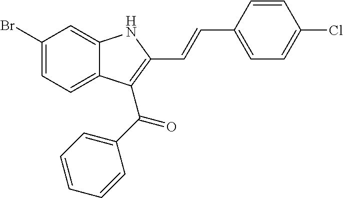 [SMILES: O=C(c1ccccc1)c1c(/C=C/c2ccc(Cl)cc2)[nH]c2cc(Br)ccc12]

Prepared according to general method V from intermediate 1-v and benzoyl chloride (32 mg, 49%). ¹H NMR (400 MHz, DMSO) δ 12.46 (s, 1H), 7.70-7.61 (m, 4H), 7.58-7.52 (m, 2H), 7.48-7.37 (m, 5H), 7.26-7.16 (m, 3H). Mass calculated for (C₂₃H₁₅BrClNO−H)⁻ 436.0, found 435.9.

### Intermediate 6-i: (E)-3-Bromo-1-(6-bromo-2-(4-chlorostyryl)-1H-indol-3-yl)propan-1-one

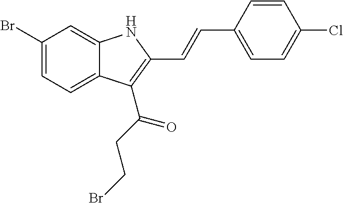 [SMILES: O=C(CCBr)c1c(/C=C/c2ccc(Cl)cc2)[nH]c2cc(Br)ccc12]

Prepared according to general method V from intermediate 1-v and 3-bromopropanoyl chloride (125 mg, 89%). ¹H NMR (400 MHz, DMSO-d₆) δ 12.43 (s, 1H), 8.00-7.90 (m, 2H), 7.68 (d, J=8.6 Hz, 2H), 7.62 (d, J=1.8 Hz, 1H), 7.54 (d, J=8.5 Hz, 2H), 7.49 (d, J=16.6 Hz, 1H), 7.36 (dd, J=8.6, 1.9 Hz, 1H), 3.84 (t, J=6.2 Hz, 2H), 3.69 (t, J=6.2 Hz, 2H).

### Compound 4: (6-Bromo-2-(4-chlorophenethyl)-1H-indol-3-yl)(tetrahydro-2H-pyran-4-yl)methanone

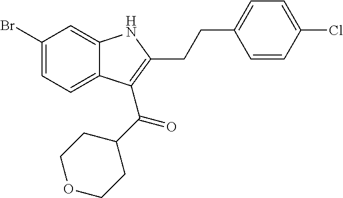 [SMILES: O=C(c1c(CCc2ccc(Cl)cc2)[nH]c2cc(Br)ccc12)C1CCOCC1]

Prepared according to general method V from intermediate 1-vi and tetrahydro-2H-pyran-4-carbonyl chloride (38 mg, 57%). ¹H NMR (400 MHz, CDCl₃) δ 8.23 (s, 1H), 7.67 (d, J=8.7 Hz, 1H), 7.48 (d, J=1.5 Hz, 1H), 7.39 (dd, J=8.6, 1.8 Hz, 1H), 7.26 (d, J=8.4 Hz, 2H), 7.11 (d, J=8.5 Hz, 2H), 4.14-4.09 (m, 2H), 3.63 (td, J=11.3, 3.1 Hz, 2H), 3.45-3.36 (m, 3H), 3.04 (t, J=7.4 Hz, 2H), 1.99-1.85 (m, 4H). Mass calculated for (C₂₂H₂₁BrClNO₂−H)⁻ 444.0, found 444.0.

### Synthesis of Compound 5

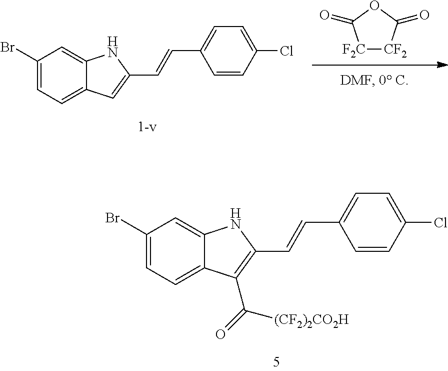

### Compound 5: (E)-4-(6-Bromo-2-(4-chlorostyryl)-1H-indol-3-yl)-2,2,3,3-tetrafluoro-4-oxobutanoic Acid

To a stirred solution of 1-v (50 mg, 0.15 mmol) in DMF (2 mL) at 0° C. under Ar was added 3,3,4,4-tetrafluorodihydrofuran-2,5-dione (90 μL, 0.84 mmol) and the mixture was stirred at 0° C. for 6 h. The reaction was quenched with 2:1 MeOH/H₂O (1 mL) and then concentrated under reduced pressure. The residue was purified by silica gel chromatography, eluting with aMeOH/DCM gradient, followed by preparative HPLC (ACN/H₂O with 0.1% formic acid) to provide compound 5 (35 mg, 46%). ¹H NMR (600 MHz, DMSO) δ 7.85 (d, J=8.6 Hz, 1H), 7.74 (d, J=16.6 Hz, 1H), 7.68 (s, 1H), 7.66-7.57 (m, 3H), 7.56-7.51 (m, 2H), 7.42 (dd, J=8.7, 1.8 Hz, 1H). Mass calculated for (C₂₀H₁₁BrClF₄NO₃−H)⁻ 504.0, found 503.8.

### Synthesis of Compound 6

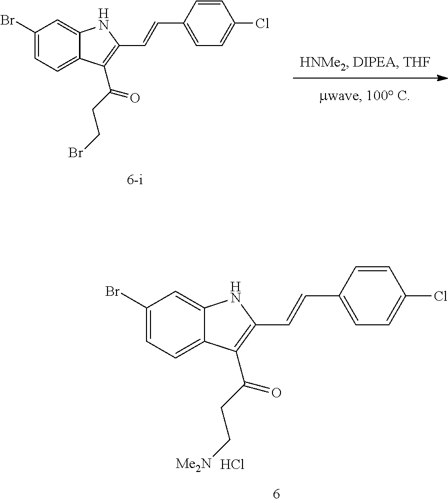 [SMILES: O=C(CCBr)c1c(/C=C/c2ccc(Cl)cc2)[nH]c2cc(Br)ccc12 ; CN(C)CCC(=O)c1c(/C=C/c2ccc(Cl)cc2)[nH]c2cc(Br)ccc12 ; Cl]

### Compound 6: (E)-1-(6-Bromo-2-(4-chlorostyryl)-1H-indol-3-yl)-3-(dimethylamino)propan-1-one Hydrochloride

A mixture of 6-i (55 mg, 0.12 mmol), dimethylamine (60 uL, 0.12 mmol) and DIPEA (28 uL, 0.16 mmol) in THF (1.0 mL) was heated with microwave at 100° C. for 1 h and then concentrated under reduced pressure. The residue was dissolved in EtOAc (2 mL) followed by addition of HCl (2 M in Et₂O, 0.1 mL, 0.2 mmol). The resulting solid was collected by filtration and washed with Et₂O (2×1 mL) to provide compound 6 mono-HCl salt as yellow solid (27 mg, 49%). ¹H NMR (400 MHz, DMSO-d₆) δ 12.65 (s, 1H), 9.59 (bs, 1H), 8.05-7.96 (m, 2H), 7.68 (d, J=8.6 Hz, 2H), 7.66 (d, J=1.7 Hz, 1H), 7.61-7.51 (m, 3H), 7.38 (dd, J=8.6, 1.9 Hz, 1H), 3.60 (t, J=6.7 Hz, 2H), 3.49 (t, J=5.8 Hz, 2H), 2.87 (d, J=4.6 Hz, 6H). Mass calculated for (C₂₁H₂₀BrClN₂O+H)⁺433.0, found 432.9.

### Synthesis of Compound 7

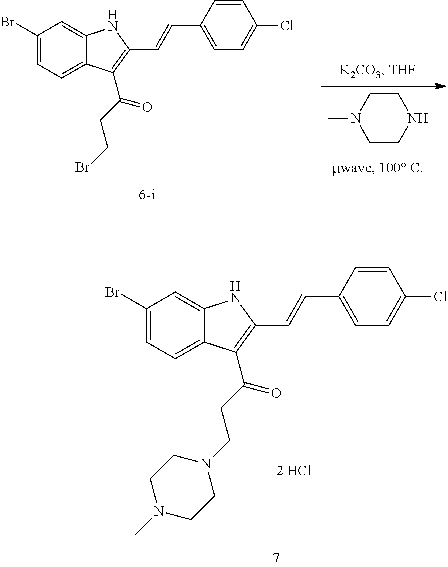

### Compound 7: (E)-1-(6-Bromo-2-(4-chlorostyryl)-1H-indol-3-yl)-3-(4-methylpiperazin-1-yl)propan-1-one Hydrochloride

A mixture of 6-i (45 mg, 0.10 mmol), 1-methylpiperazine (12 uL, 0.11 mmol) and K₂CO₃ (30 mg, 0.22 mmol) in THF (1.0 mL) was heated with microwave at 100° C. for 1 h. The reaction mixture was diluted with MeOH/EtOAc (2/8, 5 mL), filtered and concentrated under reduced pressure. The residue was dissolved in MeOH/EtOAc (1/2, 3 mL) and treated with 2M HCl (2 M in Et₂O, 0.2 mL, 0.4 mmol). The mixture was concentrated under reduced pressure and the resulting solid was triturated with MeOH (1 mL) to provide compound 7 bis-HCl salt as a yellow solid (18 mg, 36%). ¹H NMR (400 MHz, DMSO-d₆) δ 12.67 (s, 1H), 11.21 (s, 1H), 8.05-7.93 (m, 2H), 7.69 (d, J=8.6 Hz, 2H), 7.66 (d, J=1.8 Hz, 1H), 7.60 (d, J=16.7 Hz, 1H), 7.54 (d, J=8.5 Hz, 2H), 7.37 (dd, J=8.6, 1.9 Hz, 1H), 4.01-3.18 (m, 12H), 2.86 (s, 3H).

### General Method VI

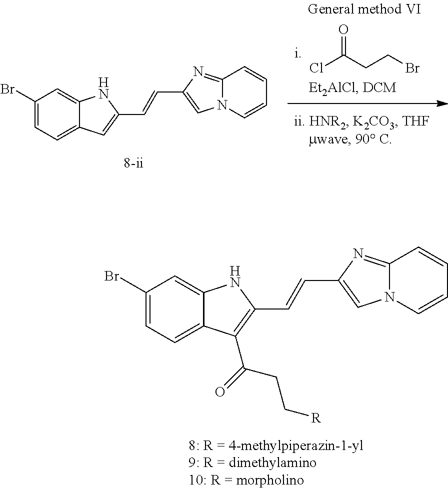

To a stirred solution of intermediate 8-ii (0.1 mmol) in DCM (2 mL) under Ar at 0° C. was added Et₂AlCl (1M in hexanes, 0.5 mmol) and the mixture was stirred at 0° C. for 30 min followed by the addition of 3-bromopropanoyl chloride (0.5 mmol) in DCM (1 mL). The mixture was stirred at 0° C. for 1 h and then diluted with DCM and EtOAc. Saturated aqueous sodium citrate (2 mL) was added and the mixture was vigorously stirred for 18 h. The layers were separated and the organic phase was dried over anhydrous Na₂SO₄, filtered and concentrated under reduced pressure. The residue was dissolved in THF (0.5 mL) followed by the addition of the corresponding amine (0.15 mmol) and K₂CO₃ (0.2 mmol). The mixture was heated with microwave at 90° C. for 45-60 min, cooled to rt and then diluted with EtOAc. The organic layer was washed with H₂O, brine, dried over anhydrous Na₂SO₄, filtered and concentrated under reduced pressure. The crude product was purified by silica gel chromatography, eluting with aneither EtOAc/hexanes or MeOH/DCM gradient, and then triturated with either Et₂O or EtOAc to provide the desired compound.

### Compound 8: (E)-1-(6-Bromo-2-(2-(imidazo[1,2-a]pyridin-2-yl)vinyl)-1H-indol-3-yl)-3-(4-methylpiperazin-1-yl)propan-1-one

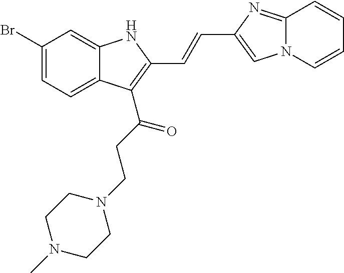 [SMILES: CN1CCN(CCC(=O)c2c(/C=C/c3cn4ccccc4n3)[nH]c3cc(Br)ccc23)CC1]

Prepared according to general method VI from intermediate 8-ii, 3-bromopropanoyl chloride and 4-methylpiperazine (23 mg, 26%). ¹H NMR (400 MHz, DMSO-d₆) δ 12.35 (s, 1H), 8.53 (d, J=6.8 Hz, 1H), 8.24-8.14 (m, 2H), 7.92 (d, J=8.7 Hz, 1H), 7.61-7.51 (m, 3H), 7.36-7.25 (m, 2H), 6.91 (t, J=6.8 Hz, 1H), 3.16 (t, J=7.1 Hz, 2H), 2.75 (t, J=7.2 Hz, 2H), 2.49-2.19 (m, 8H), 2.14 (s, 3H). Mass calculated for (C₂₅H₂₆BrN₅₀+H)⁺492.1, found 492.0.

### Compound 9: (E)-1-(6-Bromo-2-(2-(imidazo[1,2-a]pyridin-2-yl)vinyl)-1H-indol-3-yl)-3-(dimethylamino)propan-1-one

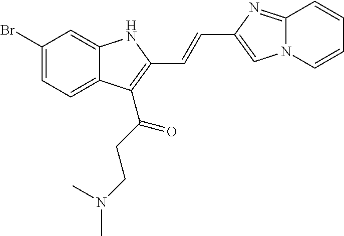 [SMILES: CN(C)CCC(=O)c1c(/C=C/c2cn3ccccc3n2)[nH]c2cc(Br)ccc12]

Prepared according to general method VI from intermediate 8-ii, 3-bromopropanoyl chloride and dimethylamine (16 mg, 18%). ¹H NMR (400 MHz, DMSO-d₆) δ 12.77 (s, 1H), 8.59-8.48 (m, 1H), 8.32-8.19 (m, 2H), 7.97 (d, J=8.7 Hz, 1H), 7.75-7.64 (m, 2H), 7.58 (d, J=9.1 Hz, 1H), 7.36 (dd, J=8.6, 1.9 Hz, 1H), 7.33-7.24 (m, 1H), 6.92 (t, J=7.0 Hz, 1H), 3.61 (t, J=6.8 Hz, 2H), 3.49 (t, J=6.7 Hz, 2H), 2.86 (s, 6H). Mass calculated for (C₂₂H₂₁BrN₄₀+H)⁺437.1, found 437.0.

### Compound 10: (E)-1-(6-Bromo-2-(2-(imidazo[1,2-a]pyridin-2-yl)vinyl)-1H-indol-3-yl)-3-morpholinopropan-1-one

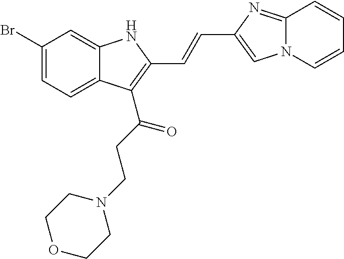 [SMILES: O=C(CCN1CCOCC1)c1c(/C=C/c2cn3ccccc3n2)[nH]c2cc(Br)ccc12]

Prepared according to general method VI from intermediate 8-ii, 3-bromopropanoyl chloride and morpholine (13 mg, 14%). ¹H NMR (400 MHz, DMSO-d₆) δ 12.34 (s, 1H), 8.53 (d, J=6.7 Hz, 1H), 8.24-8.14 (m, 2H), 7.93 (d, J=8.6 Hz, 1H), 7.63-7.48 (m, 3H), 7.33 (dd, J=8.6, 1.9 Hz, 1H), 7.32-7.26 (m, 1H), 6.91 (td, J=6.8, 1.2 Hz, 1H), 3.62-3.53 (m, 4H), 3.19 (t, J=7.2 Hz, 2H), 2.76 (bs, 2H), 2.45 (s, 4H). Mass calculated for (C₂₄H₂₃BrN₄O₂+H)⁺481.1, found 480.9.

### General Method VII

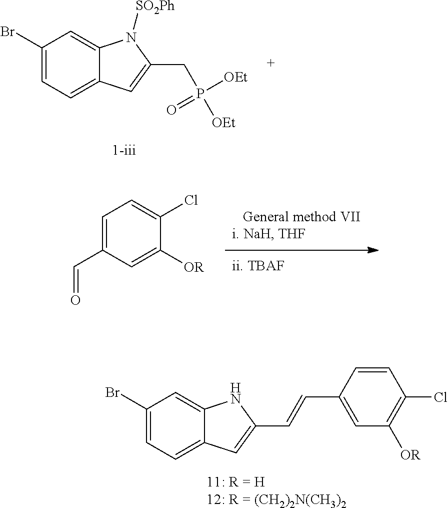

NaH (1.5 equiv) was added to a cold (0° C.) stirring solution of phosphonate 1-iii under N₂. After 30 min, the corresponding aldehyde (1.1 equiv) was added at ambient temperature. The mixture was stirred for 3-6 h followed by the addition of 1.0M solution of tetrabutylammonium fluoride (5 equiv). The resulting mixture was further stirred at ambient temperature for 16 h and then partitioned between H₂O and EtOAc. The organic layer was washed with H₂O (1×) and brine (1×). The combined organics was dried over MgSO₄, filtered and concentrated in vacuo. The crude product was purified by silica gel chromatography, eluting an EtOAc/hexanes or MeOH/DCM gradient, to provide the desired intermediate.

### Compound 11: (E)-5-(2-(6-bromo-1H-indol-2-yl)vinyl)-2-chlorophenol

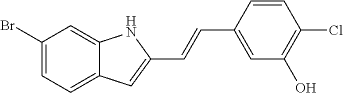 [SMILES: Oc1cc(/C=C/c2cc3ccc(Br)cc3[nH]2)ccc1Cl]

Prepared according to general method VII from intermediate 1-iii and 4-chloro-3-hydroxybenzaldehyde (21 mg, 40%). ¹H NMR (400 MHz, CDCl₃) δ 8.26 (s, 1H), 7.52 (t, J=1.1 Hz, 1H), 7.45 (d, J=8.4 Hz, 1H), 7.33 (d, J=8.3 Hz, 1H), 7.22 (dd, J=8.4, 1.7 Hz, 1H), 7.18 (d, J=2.1 Hz, 1H), 7.11-7.01 (m, 2H), 6.84 (d, J=16.5 Hz, 1H), 6.64-6.59 (m, 1H), 5.59 (s, 1H). Mass calculated for (C₁₆H₁₁BrClNO−H)⁻ 346.0, found 346.5.

### Compound 12: (E)-2-(5-(2-(6-bromo-1H-indol-2-yl)vinyl)-2-chlorophenoxy)-N,N-dimethylethanamine

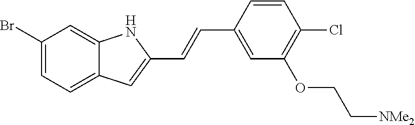 [SMILES: CN(C)CCOc1cc(/C=C/c2cc3ccc(Br)cc3[nH]2)ccc1Cl]

Prepared according to general method VII from intermediate 1-iii and 4-chloro-3-(2-(dimethylamino)ethoxy)benzaldehyde (30 mg, 36%). ¹H NMR (400 MHz, CDCl₃) δ 8.42 (s, 1H), 7.53 (t, J=1.2 Hz, 1H), 7.46 (d, J=8.4 Hz, 1H), 7.36 (d, J=8.1 Hz, 1H), 7.22 (dd, J=8.4, 1.7 Hz, 1H), 7.11-7.03 (m, 3H), 6.88 (d, J=16.5 Hz, 1H), 6.61 (d, J=1.9 Hz, 1H), 4.26 (t, J=5.7 Hz, 2H), 2.93 (t, J=5.7 Hz, 2H), 2.48 (s, 6H). Mass calculated for (C₂₀H₂₀BrClN₂O+H)⁺419.0, found 419.4.

### General Method VIII

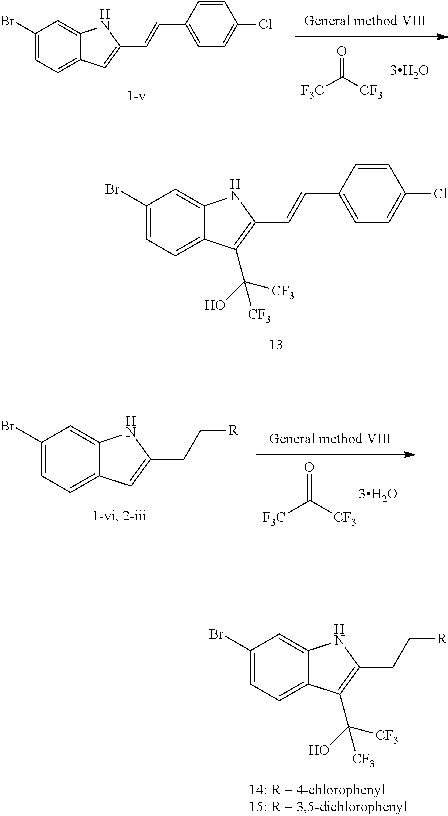

A mixture of the corresponding indole (1.0 mmol) and hexafluoroacetone trihydrate (10.0 mmol) was heated in a sealed tube at 105° C. for 20 h and then diluted with EtOAc. The mixture was washed with H₂O, brine, dried over anhydrous Na₂SO₄, filtered and concentrated under reduced pressure. The crude product was purified by silica gel chromatography, eluting with an EtOAc/hexanes or MeOH/DCM gradient, to provide the desired product.

### Compound 13: (E)-2-(6-Bromo-2-(4-chlorostyryl)-1H-indol-3-yl)-1,1,1,3,3,3-hexafluoropropan-2-ol

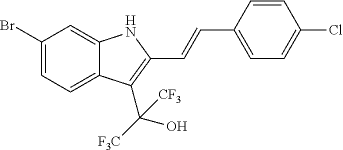 [SMILES: OC(c1c(/C=C/c2ccc(Cl)cc2)[nH]c2cc(Br)ccc12)(C(F)(F)F)C(F)(F)F]

Prepared according to general method VIII from intermediate 1-v. (50 mg, 61%). ¹H NMR (600 MHz, CDCl₃) δ 8.51 (s, 1H), 7.93-7.60 (m, 2H), 7.55 (d, J=1.8 Hz, 1H), 7.46 (d, J=8.5 Hz, 2H), 7.39 (d, J=8.5 Hz, 2H), 7.29 (dd, J=8.9, 1.8 Hz, 1H), 6.92 (d, J=16.6 Hz, 1H), 3.57 (s, 1H). Mass calculated for (C₁₉H₁₁BrClF₆NO−H)⁻ 498.0, found 498.0.

### Compound 14: 2-(6-Bromo-2-(4-chlorophenethyl)-1H-indol-3-yl)-1,1,1,3,3,3-hexafluoropropan-2-ol

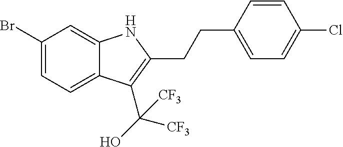 [SMILES: OC(c1c(CCc2ccc(Cl)cc2)[nH]c2cc(Br)ccc12)(C(F)(F)F)C(F)(F)F]

Prepared according to general method VIII from intermediate 1-vi. (65 mg, 75%). H NMR (600 MHz, DMSO) δ 11.66 (s, 1H), 8.45 (s, 1H), 7.53 (d, J=1.8 Hz, 1H), 7.38 (d, J=8.4 Hz, 2H), 7.26 (d, J=8.3 Hz, 2H), 7.17 (dd, J=8.9, 1.9 Hz, 1H), 3.23 (s, 2H), 2.97 (m, 2H). Mass calculated for (C₁₉H₁₃BrCl F₆NO−H)⁻ 498.0, found 498.2.

### Compound 15: 2-(6-Bromo-2-(3,5-dichlorophenethyl)-1H-indol-3-yl)-1,1,1,3,3,3-hexafluoropropan-2-ol

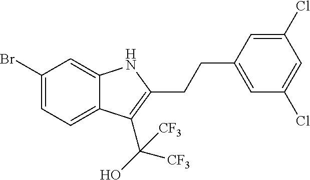 [SMILES: OC(c1c(CCc2cc(Cl)cc(Cl)c2)[nH]c2cc(Br)ccc12)(C(F)(F)F)C(F)(F)F]

Prepared according to general method VIII from intermediate 2-iii. (50 mg, 59%). ¹H NMR (600 MHz, DMSO) δ 11.64 (s, 1H), 8.48 (s, 1H), 7.55 (m, 3H), 7.29 (s, 2H), 7.19 (s, 1H), 3.25 (s, 2H), 2.99 (s, 1H). Mass calculated for (C₁₉H₁₂BrCl₂ F₆NO−H)⁻ 533.9, found 533.8.

### General Method IX

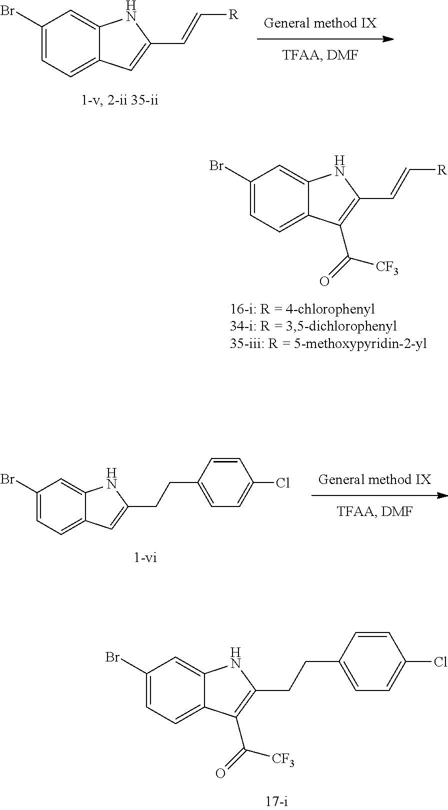

To a stirred solution of the corresponding indole (1.0 mmol) in DMF (10 mL) under Ar at 0° C. was added TFAA (2.0 mmol) and the mixture was stirred at 0° C. for 2-6 h. The reaction was quenched with H₂O and then diluted with EtOAc. The organic layer was washed with H₂O, brine, dried over anhydrous Na₂SO₄, filtered and concentrated under reduced pressure. The crude product was purified by silica gel chromatography, eluting with an EtOAc/hexanes gradient, to provide the desired adduct.

### Intermediate 16-i: (E)-1-(6-Bromo-2-(4-chlorostyryl)-1H-indol-3-yl)-2,2,2-trifluoroethanone

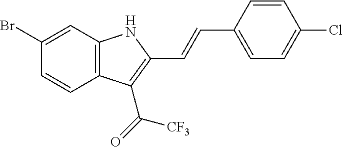 [SMILES: O=C(c1c(/C=C/c2ccc(Cl)cc2)[nH]c2cc(Br)ccc12)C(F)(F)F]

Prepared according to general method IX from intermediate 1-v (165 mg, 80%). ¹H NMR (600 MHz, CDCl₃) δ 9.18 (s, 1H), 7.96-7.89 (m, 2H), 7.59 (d, J=1.5 Hz, 1H), 7.51 (d, J=8.4 Hz, 2H), 7.42 (dd, J=8.7, 1.8 Hz, 1H), 7.39 (d, J=8.4 Hz, 2H), 7.20 (d, J=16.7 Hz, 1H). Mass calculated for (C₁₈H₁₀BrClF₃NO−H)⁻ 428.0, found 427.9.

### Intermediate 34-i: (E)-1-(6-Bromo-2-(3,5-dichlorostyryl)-1H-indol-3-yl)-2,2,2-trifluoroethanone

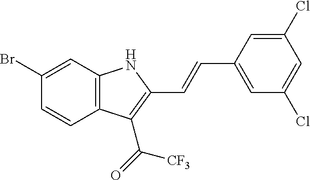 [SMILES: O=C(c1c(/C=C/c2cc(Cl)cc(Cl)c2)[nH]c2cc(Br)ccc12)C(F)(F)F]

Prepared according to general method IX from intermediate 2-ii (495 mg, 89%). ¹H NMR (500 MHz, DMSO) δ 13.14 (s, 1H), 7.87 (d, J=16.5 Hz, 1H), 7.80 (d, J=8.6 Hz, 1H), 7.73 (d, J=1.8 Hz, 1H), 7.72 (d, J=1.8 Hz, 2H), 7.67 (t, J=1.8 Hz, 1H), 7.54 (d, J=16.6 Hz, 1H), 7.47 (dd, J=8.8, 1.8 Hz, 1H). Mass calculated for (C₁₈H₉BrCl₂F₃NO−H)⁻ 461.9, found 461.9.

### Intermediate 35-iii: (E)-1-(6-Bromo-2-(2-(5-methoxypyridin-2-yl)vinyl)-1H-indol-3-yl)-2,2,2-trifluoroethanone

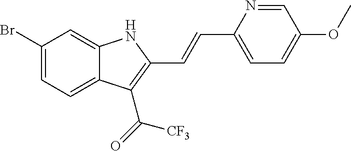 [SMILES: COc1ccc(/C=C/c2[nH]c3cc(Br)ccc3c2C(=O)C(F)(F)F)nc1]

Prepared according to general method IX from intermediate 35-ii (32 mg, 50%). ¹H NMR (400 MHz, DMSO) δ 13.05 (s, 1H), 8.44 (d, J=2.9 Hz, 1H), 8.16 (d, J=16.1 Hz, 1H), 7.82 (d, J=8.7 Hz, 1H), 7.71-7.63 (m, 2H), 7.59 (d, J=8.6 Hz, 1H), 7.51-7.42 (m, 2H), 3.90 (s, 3H). Mass calculated for (C₁₈H₁₂BrF₃N₂O₂+H)⁺425.0, found 425.0.

### Intermediate 17-i: 1-(6-Bromo-2-(4-chlorophenethyl)-1H-indol-3-yl)-2,2,2-trifluoroethanone

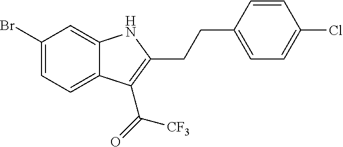 [SMILES: O=C(c1c(CCc2ccc(Cl)cc2)[nH]c2cc(Br)ccc12)C(F)(F)F]

Prepared according to general method IX from intermediate 1-vi (250 mg, 69%). ¹H NMR (600 MHz, DMSO) δ 7.75 (d, J=8.5 Hz, 1H), 7.70 (d, J=1.8 Hz, 1H), 7.43 (dd, J=8.7, 1.9 Hz, 1H), 7.37 (d, J=8.3 Hz, 2H), 7.26 (d, J=8.4 Hz, 2H), 3.40-3.33 (m, 2H), 3.01-2.95 (m, 2H). Mass calculated for (C₁₈H₁₂BrClF₃NO−H)⁻ 430.0, found 430.0.

### General Method X

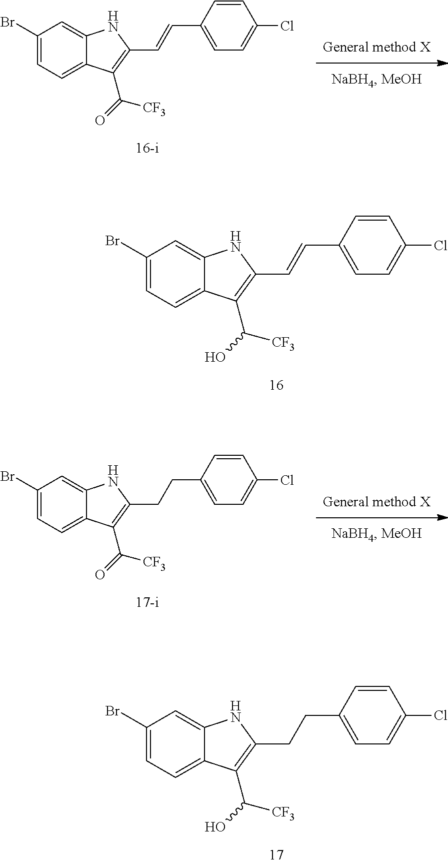 [SMILES: O=C(c1c(/C=C/c2ccc(Cl)cc2)[nH]c2cc(Br)ccc12)C(F)(F)F ; OC(c1c(/C=C/c2ccc(Cl)cc2)[nH]c2cc(Br)ccc12)C(F)(F)F ; O=C(c1c(CCc2ccc(Cl)cc2)[nH]c2cc(Br)ccc12)C(F)(F)F ; OC(c1c(CCc2ccc(Cl)cc2)[nH]c2cc(Br)ccc12)C(F)(F)F]

To a stirred solution of the trifluoromethyl ketone intermediate (1.0 mmol) in MeOH (30 mL) at 0° C. was added NaBH₄ (1.8 mmol) and the mixture was stirred for 90 min. The reaction was quenched with H₂O and then diluted with EtOAc. The organic phase was washed with H₂O, brine, dried over anhydrous Na₂SO₄, filtered and concentrated under reduced pressure. The crude product was purified by silica gel chromatography, eluting with an EtOAc/hexanes gradient, to provide the desired product.

### Compound 16: (E)-1-(6-Bromo-2-(4-chlorostyryl)-1H-indol-3-yl)-2,2,2-trifluoroethanol

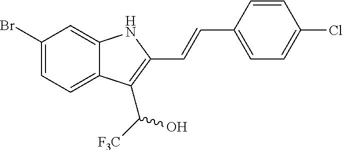 [SMILES: OC(c1c(/C=C/c2ccc(Cl)cc2)[nH]c2cc(Br)ccc12)C(F)(F)F]

Prepared according to general method X from intermediate 16-i (26 mg, 67%). ¹H NMR (600 MHz, CDCl₃) δ 8.37 (s, 1H), 7.65 (d, J=8.5 Hz, 1H), 7.52 (d, J=1.7 Hz, 1H), 7.47 (d, J=8.4 Hz, 2H), 7.39 (d, J=8.5 Hz, 2H), 7.29-7.23 (m,2H), 6.91 (d, J=16.5 Hz, 1H), 5.54-5.49 (m,1H), 2.65 (d, J=3.7 Hz, 1H), 5.42-5.33 (m, 1H). Mass calculated for (C₁₈H₁₂BrClF₃NO+H)⁺ 431.0, found 431.9.

### Compound 17: 1-(6-Bromo-2-(4-chlorophenyl)-1H-indol-3-yl)-2,2,2-trifluoroethanol

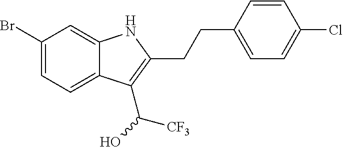 [SMILES: OC(c1c(CCc2ccc(Cl)cc2)[nH]c2cc(Br)ccc12)C(F)(F)F]

Prepared according to general method X from intermediate 17-i (39 mg, 93%). ¹H NMR (600 MHz, DMSO) δ 11.35 (s, 1H), 7.59 (d, J=8.4 Hz, 1H), 7.49 (d, J=1.7 Hz, 1H), 7.36 (d, J=8.4 Hz, 2H), 7.30 (d, J=8.4 Hz, 2H), 7.11 (dd, J=8.5, 1.8 Hz, 1H), 6.54 (d, J=4.6 Hz, 1H), 5.42-5.33 (m, 1H), 3.08-3.02 (m, 2H), 2.96-2.91 (m, 2H). Mass calculated for (C₁₈H₁₄BrClF₃NO−H)⁻ 432.0, found 432.0.

### Synthesis of Compound 18

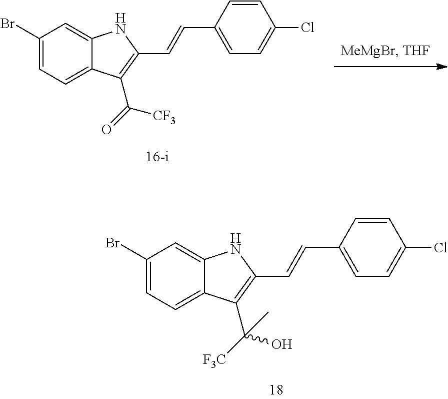 [SMILES: O=C(c1c(/C=C/c2ccc(Cl)cc2)[nH]c2cc(Br)ccc12)C(F)(F)F ; CC(O)(c1c(/C=C/c2ccc(Cl)cc2)[nH]c2cc(Br)ccc12)C(F)(F)F]

### Compound 18: (E)-2-(6-Bromo-2-(4-chlorostyryl)-1H-indol-3-yl)-1,1,1-trifluoropropan-2-ol

To a stirred solution of compound 16-i (1.0 mmol) in THF (20 mL) at °0 C. under Ar was added MeMgBr (3M in Et₂O, 3.6 mmol) and the mixture was stirred for 3 h. The reaction was quenched with saturated aqueous NH₄Cl and then diluted with EtOAc. The organic phase was washed with H₂O, brine, dried over anhydrous Na₂SO₄, filtered and concentrated under reduced pressure. The crude product was purified by preparative HPLC (ACN/H₂O with 0.1% formic acid) to provide compound 18 (18 mg, 46%). ¹H NMR (600 MHz, CDCl₃) δ 8.38 (s, 1H), 7.80 (d, J=16.6 Hz, 1H), 7.66 (d, J=8.7 Hz, 1H), 7.51 (d, J=1.6 Hz, 1H), 7.45 (d, J=8.5 Hz, 2H), 7.37 (d, J=8.5 Hz, 2H), 7.24 (dd, J=8.7, 1.8 Hz, 1H), 6.85 (d, J=16.6 Hz, 1H), 2.50 (bs, 1H), 2.08 (s, 3H). Mass calculated for (C₁₉H₁₄BrClF₃NO+H)⁺446.0, found 445.8.

### General Method XI

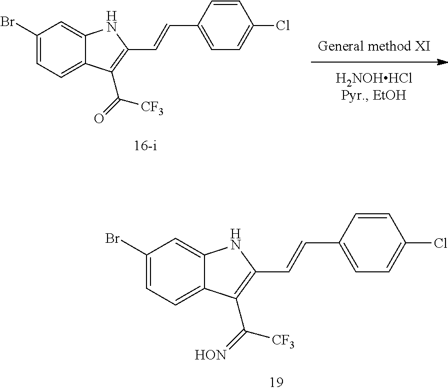

A mixture of corresponding trifluoromethyl ketone (1.0 mmol), hydroxylamine hydrochloride (3.5 mmol) and pyridine (15.0 mmol) in EtOH (18 mL) was refluxed for 4 h and then diluted with EtOAc. The mixture was washed with 1M aqueous HCl, H₂O, brine, dried over anhydrous Na₂SO₄, filtered and concentrated under reduced pressure. The crude product was purified by silica gel chromatography, eluting with an EtOAc/hexanes gradient, to provide the desired product.

### Compound 19: 1-(6-Bromo-2-(4-chlorostyryl)-1H-indol-3-yl)-2,2,2-trifluoroethanone Oxime

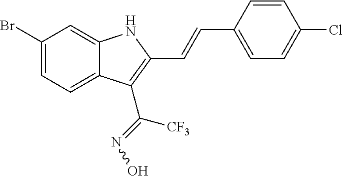 [SMILES: ON=C(c1c(/C=C/c2ccc(Cl)cc2)[nH]c2cc(Br)ccc12)C(F)(F)F]

Prepared according to general method XI from intermediate 16-i (30 mg, 83%). Present as 2:1 mixture of isomers.

Major isomer: ¹H NMR (600 MHz, DMSO) δ 12.11 (s, 1H), 7.60 (bs, 1H), 7.58 (d, J=8.5 Hz, 2H), 7.49 (d, J=8.4 Hz, 2H), 7.36 (d, J=16.8 Hz, 1H), 7.34 (d, J=10.3 Hz, 1H), 7.25 (dd, J=8.5, 1.8 Hz, 1H), 7.10 (d, J=16.5 Hz, 1H).

Minor isomer: ¹H NMR (600 MHz, DMSO) δ 12.12 (s, 1H), 7.60 (s, 1H), 7.58 (d, J=8.5 Hz, 2H), 7.48 (d, J=7.6 Hz, 2H), 7.32 (d, J=17.1 Hz, 1H), 7.25 (d, J=8.9 Hz, 1H), 7.22 (dd, J=8.5, 1.7 Hz, 1H), 6.90 (d, J=16.5 Hz, 1H).

Mass calculated for (C₁₈H₁₁BrClF₃N₂O+H)⁺445.0, found 445.3.

### General Method XII

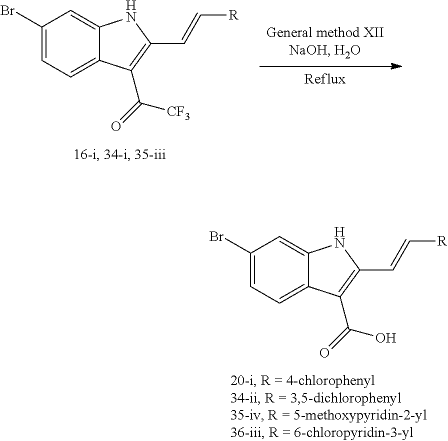

A suspension of the corresponding trifluoromethyl ketone in 20% NaOH aqueous solution (30 mL) was refluxed for 3-16 h. The mixture was cooled to rt and then acidified with 15% aqueous HCl to pH ˜3. The mixture was extracted with EtOAc (×2) and the combined organic phase was concentrated under reduced pressure. The crude product was purified by silica gel chromatography, eluting with as EtOAc/hexanes or MeOH/DCM gradient, to provide the desired intermediate.

### Intermediate 20-i: (E)-6-Bromo-2-(4-chlorostyryl)-1H-indole-3-carboxylic Acid

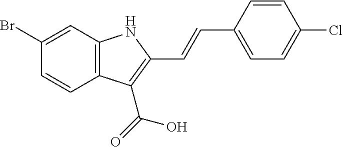 [SMILES: O=C(O)c1c(/C=C/c2ccc(Cl)cc2)[nH]c2cc(Br)ccc12]

Prepared according to general method XII from intermediate 16-i (1.32 g, 72%). ¹H NMR (400 MHz, DMSO) δ 12.52 (s, 1H), 12.26 (s, 1H), 8.04 (d, J=16.9 Hz, 1H), 7.95 (d, J=8.6 Hz, 1H), 7.64-7.56 (m, 3H), 7.54-7.50 (m, 2H), 7.45 (d, J=16.8 Hz, 1H), 7.28 (dd, J=8.6, 1.8 Hz, 1H). Mass calculated for (C₁₇H₁₁BrClNO₂−H)⁻ 374.0, found 374.0.

### Intermediate 34-ii: (E)-6-Bromo-2-(3,5-dichlorostyryl)-1H-indole-3-carboxylic Acid

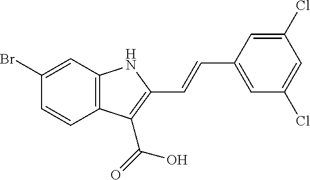 [SMILES: O=C(O)c1c(/C=C/c2cc(Cl)cc(Cl)c2)[nH]c2cc(Br)ccc12]

Prepared according to general method XII from intermediate 34-i (130 mg, 73%). ¹H NMR (600 MHz, DMSO) δ 12.31 (s, 1H), 8.06 (d, J=16.8 Hz, 1H), 7.96 (d, J=8.6 Hz, 1H), 7.62-7.58 (m, 4H), 7.38 (d, J=16.7 Hz, 1H), 7.30 (dd, J=8.6, 1.8 Hz, 1H). Mass calculated for (C₁₇H₁₀BrCl₂NO₂−H)⁻ 407.9, found 408.1.

### Intermediate 35-iv: (E)-6-Bromo-2-(2-(5-methoxypyridin-2-yl)vinyl)-1H-indole-3-carboxylic Acid

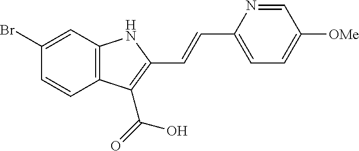 [SMILES: COc1ccc(/C=C/c2[nH]c3cc(Br)ccc3c2C(=O)O)nc1]

Prepared according to general method XII from intermediate 35-iii (74 mg, 58%). ¹H NMR (400 MHz, DMSO) δ 12.48 (bs, 1H), 12.25 (s, 1H), 8.40 (d, J=2.8 Hz, 1H), 8.33 (d, J=16.5 Hz, 1H), 7.96 (d, J=8.6 Hz, 1H), 7.57 (d, J=1.6 Hz, 1H), 7.53-7.41 (m, 3H), 7.28 (dd, J=8.6, 1.7 Hz, 1H), 3.89 (s, 3H). Mass calculated for (C₁₇H₁₃BrN₂O₃−H)⁻ 371.0, found 371.0.

### Intermediate 36-iii: (E)-6-Bromo-2-(2-(6-chloropyridin-3-yl)vinyl)-1H-indole-3-carboxylic Acid

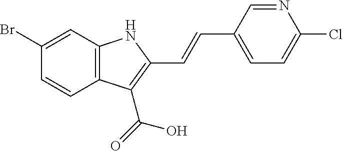 [SMILES: O=C(O)c1c(/C=C/c2ccc(Cl)nc2)[nH]c2cc(Br)ccc12]

Prepared according to general method IX and XII from intermediate 36-ii (245 mg, 53%). ¹H NMR (400 MHz, DMSO) δ 12.52 (s, 1H), 12.34 (s, 1H), 8.57 (d, J=2.5 Hz, 1H), 8.14-8.06 (m, 2H), 7.96 (d, J=8.6 Hz, 1H), 7.62-7.57 (m, 2H), 7.45 (d, J=16.9 Hz, 1H), 7.30 (dd, J=8.6, 1.8 Hz, 1H). Mass calculated for (C₁₆H₁₀BrClN₂O₂+H)⁺379.0, found 379.1.

### General Method XIII and XIV

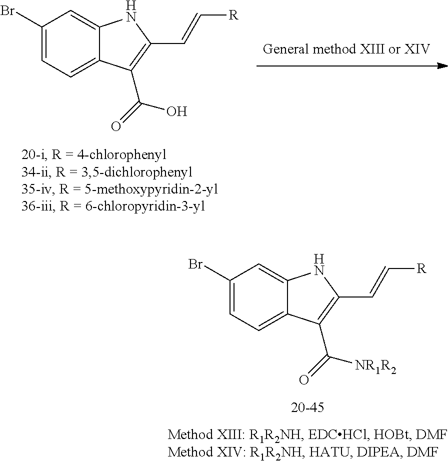

### General Method XIII

A solution of the corresponding carboxylic acid (1.0 mmol), EDC-HCl (1.5 mmol), HOBt (1.5 mmol) and the corresponding amine (2.5 mmol) in DMF (15 mL) was stirred at rt for 16 h and then diluted with EtOAc. The organic layer was washed with H₂O, brine, dried over anhydrous Na₂SO₄, filtered and concentrated under reduced pressure. The crude product was purified by silica gel chromatography, eluting with an EtOAc/hexanes or MeOH/DCM gradient, to provide the desired compound.

### General Method XIV

A solution of the corresponding carboxylic acid (1.0 mmol), DIPEA (2.2 mmol) and HATU (1.1 mmol) in DMF (25 mL) was stirred at rt for 5 min followed by the addition of the corresponding amine (1.4 mmol). The organic layer was washed with H₂O, brine, dried over anhydrous Na₂SO₄, filtered and concentrated under reduced pressure. The crude product was purified by silica gel chromatography, eluting with an EtOAc/hexanes or MeOH/DCM gradient, to provide the desired compound.

### Compound 20: (E)-6-Bromo-2-(4-chlorostyryl)-N-methyl-1H-indole-3-carboxamide

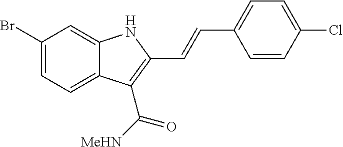 [SMILES: CNC(=O)c1c(/C=C/c2ccc(Cl)cc2)[nH]c2cc(Br)ccc12]

Prepared according to general method XIII from intermediate 20-i and methylamine (44 mg, 85%). ¹H NMR (400 MHz, DMSO) δ 11.97 (s, 1H), 7.86-7.79 (m, 1H), 7.74 (d, J=16.8 Hz, 1H), 7.70 (d, J=8.8 Hz, 1H), 7.58 (d, J=8.5 Hz, 2H), 7.54 (d, J=1.5 Hz, 1H), 7.49 (d, J=8.6 Hz, 2H), 7.32 (d, J=16.7 Hz, 1H), 7.23 (dd, J=8.6, 1.8 Hz, 1H), 2.83 (d, J=4.6 Hz, 3H). Mass calculated for (C₁₈H₁₄BrClN₂O−H)⁻ 387.0, found 387.0.

### Compound 21: (E)-6-Bromo-2-(4-chlorostyryl)-N,N-dimethyl-1H-indole-3-carboxamide

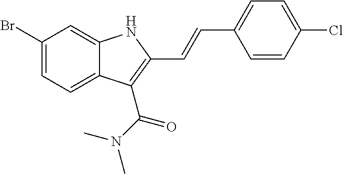 [SMILES: CN(C)C(=O)c1c(/C=C/c2ccc(Cl)cc2)[nH]c2cc(Br)ccc12]

Prepared according to general method XIII from intermediate 20-i and dimethylamine (45 mg, 70%). ¹H NMR (400 MHz, DMSO) δ 11.93 (s, 1H), 7.58 (d, J=8.6 Hz, 2H), 7.56 (d, J=1.5 Hz, 1H), 7.46 (d, J=8.6 Hz, 2H), 7.36 (d, J=8.5 Hz, 1H), 7.29 (d, J=16.6 Hz, 1H), 7.22-7.16 (m, 2H), 2.99 (bs, 6H). Mass calculated for (C₁₉H₁₆BrClN₂O+H)⁺ 405.0, found 405.1.

### Compound 22: (E)-(6-Bromo-2-(4-chlorostyryl)-1H-indol-3-yl)(morpholino)methanone

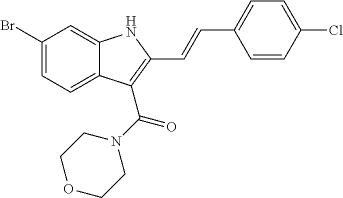 [SMILES: O=C(c1c(/C=C/c2ccc(Cl)cc2)[nH]c2cc(Br)ccc12)N1CCOCC1]

Prepared according to general method XIII from intermediate 20-i and morpholine (18 mg, 51%). ¹H NMR (600 MHz, DMSO) δ 12.00 (s, 1H), 7.61 (d, J=8.5 Hz, 2H), 7.58 (d, J=1.5 Hz, 1H), 7.49 (d, J=8.5 Hz, 2H), 7.44 (d, J=8.5 Hz, 1H), 7.31 (d, J=16.6 Hz, 1H), 7.26 (d, J=16.8 Hz, 1H), 7.23 (dd, J=8.5, 1.8 Hz, 1H), 3.63 (bs, 4H), 3.52 (bs, 4H). Mass calculated for (C₂₁H₁₈BrClN₂O₂+H)⁺446.9, found 447.0.

### Compound 23: (E)-6-Bromo-2-(4-chlorostyryl)-N-(2-(dimethylamino)ethyl)-1H-indole-3-carboxamide

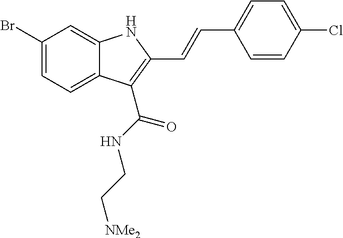 [SMILES: CN(C)CCNC(=O)c1c(/C=C/c2ccc(Cl)cc2)[nH]c2cc(Br)ccc12]

Prepared according to general method XIII from intermediate 20-i and N¹,N¹-dimethylethane-1,2-diamine (15 mg, 42%). ¹H NMR (600 MHz, DMSO) δ 11.97 (s, 1H), 7.79-7.73 (m, 2H), 7.70 (d, J=8.6 Hz, 1H), 7.60 (d, J=8.5 Hz, 2H), 7.56 (d, J=1.6 Hz, 1H), 7.50 (d, J=8.5 Hz, 2H), 7.32 (d, J=16.7 Hz, 1H), 7.24 (dd, J=8.5, 1.7 Hz, 1H), 3.42 (q, J=6.4 Hz, 2H), 2.47 (t, J=6.6 Hz, 2H), 2.24 (s, 6H). Mass calculated for (C₂₁H₂₁BrClN₃O+H)⁺448.0, found 448.1.

### Compound 24: (E)-6-Bromo-2-(4-chlorostyryl)-N-(2-(dimethylamino)ethyl)-N-methyl-1H-indole-3-carboxamide

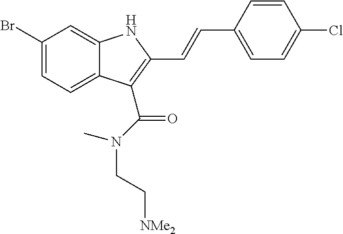 [SMILES: CN(C)CCN(C)C(=O)c1c(/C=C/c2ccc(Cl)cc2)[nH]c2cc(Br)ccc12]

Prepared according to general method XIII from intermediate 20-i and N¹,N¹,N²-trimethylethane-1,2-diamine (30 mg, 38%). ¹H NMR (600 MHz, DMSO) δ 11.90 (s, 1H), 7.68-7.53 (m, 3H), 7.49 (d, J=7.8 Hz, 2H), 7.37 (d, J=7.7 Hz, 1H), 7.30 (d, J=16.6 Hz, 1H), 7.20 (m, 2H), 3.63 (s, 2H), 2.97 (s, 3H), 2.28 (s, 6H), 1.87 (s, 2H). Mass calculated for (C₂₂H₂₃BrCl₂ N₃O+H)⁺ 462.1, found 462.0.

### Compound 25: (E)-(6-Bromo-2-(4-chlorostyryl)-1H-indol-3-yl)(piperazin-1-yl)methanone

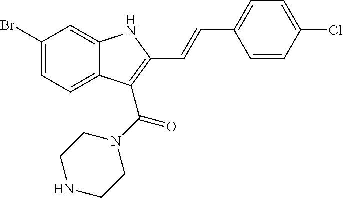 [SMILES: O=C(c1c(/C=C/c2ccc(Cl)cc2)[nH]c2cc(Br)ccc12)N1CCNCC1]

Prepared according to general method XIII from intermediate 20-i and piperazine (9 mg, 26%). ¹H NMR (400 MHz, DMSO) δ 11.95 (s, 1H), 7.59 (d, J=8.6 Hz, 2H), 7.56 (d, J=1.5 Hz, 1H), 7.48 (d, J=8.6 Hz, 2H), 7.40 (d, J=8.5 Hz, 1H), 7.30 (d, J=16.6 Hz, 1H), 7.25-7.18 (m, 2H), 3.44 (bs, J=21.8 Hz, 4H), 2.71 (bs, J=11.0 Hz, 4H). Mass calculated for (C₂₁H₁₉BrClN₃O+H)⁺446.0, found 446.0.

### Compound 26: (E)-6-Bromo-2-(4-chlorostyryl)-N-(quinuclidin-3-yl)-1H-indole-3-carboxamide

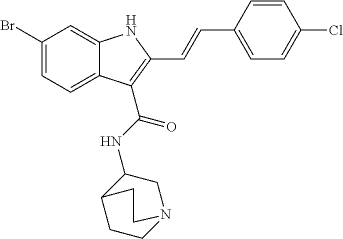 [SMILES: O=C(NC1CN2CCC1CC2)c1c(/C=C/c2ccc(Cl)cc2)[nH]c2cc(Br)ccc12]

Prepared according to general method XIII from intermediate 20-i and quinuclidin-3-amine hydrochloride (25 mg, 66%). ¹H NMR (600 MHz, MeOD) δ 7.69-7.50 (m, 5H), 7.40 (d, J=8.2 Hz, 2H), 7.29-7.19 (m, 2H), 4.32 (bs, 1H), 3.56 (t, J=11.3 Hz, 1H), 3.21-2.94 (m, 5H), 2.24 (s, 1H), 2.15-2.07 (m, 1H), 1.93 (t, J=6.8 Hz, 2H), 1.75 (t, J=12.1 Hz, 1H). Mass calculated for (C₂₄H₂₃BrClN₃O+H)⁺ 486.1, found 486.1.

### Compound 27: (E)-N-Benzyl-6-bromo-2-(4-chlorostyryl)-1H-indole-3-carboxamide

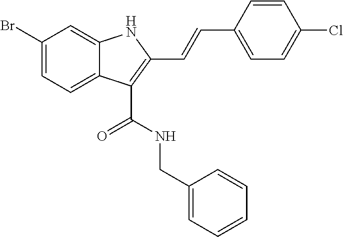 [SMILES: O=C(NCc1ccccc1)c1c(/C=C/c2ccc(Cl)cc2)[nH]c2cc(Br)ccc12]

Prepared according to general method XIII from intermediate 20-i and benzylamine. ¹H NMR (400 MHz, MeOD) δ 7.69-7.62 (m, 2H), 7.57 (d, J=1.4 Hz, 1H), 7.51-7.45 (m, 4H), 7.43-7.36 (m, 4H), 7.33 (d, J=7.2 Hz, 1H), 7.26 (s, 1H), 7.24-7.20 (m, 1H), 4.68 (s, 2H). Mass calculated for (C₂₄H₁₈BrClN₂O−H)⁻ 463.03, found 463.3.

### Compound 28: (E)-6-Bromo-2-(4-chlorostyryl)-N-(2-hydroxyethyl)-1H-indole-3-carboxamide

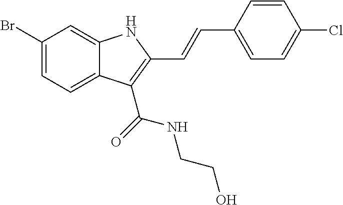 [SMILES: O=C(NCCO)c1c(/C=C/c2ccc(Cl)cc2)[nH]c2cc(Br)ccc12]

Prepared according to general method XIV from intermediate 20-i and 2-aminoethanol (30 mg, 90%). ¹H NMR (400 MHz, DMSO) δ 11.98 (s, 1H), 7.83 (t, J=5.5 Hz, 1H), 7.76 (d, J=16.7 Hz, 1H), 7.71 (d, J=8.6 Hz, 1H), 7.60 (d, J=8.5 Hz, 2H), 7.56 (d, J=1.6 Hz, 1H), 7.49 (d, J=8.5 Hz, 2H), 7.33 (d, J=16.7 Hz, 1H), 7.24 (dd, J=8.6, 1.7 Hz, 1H), 4.79 (t, J=5.4 Hz, 1H), 3.59 (q, J=5.9 Hz, 2H), 3.41 (q, J=6.0 Hz, 2H). Mass calculated for (C₁₉H₁₆BrClN₂O₂−H)⁻ 419.0, found 419.1.

### Compound 29: (E)-6-Bromo-2-(4-chlorostyryl)-N-(2-morpholinoethyl)-1H-indole-3-carboxamide

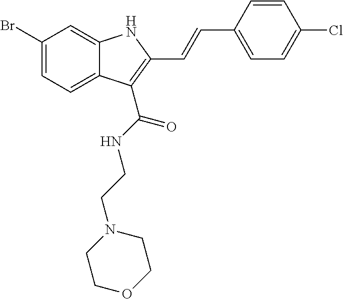 [SMILES: O=C(NCCN1CCOCC1)c1c(/C=C/c2ccc(Cl)cc2)[nH]c2cc(Br)ccc12]

Prepared according to general method XIV from intermediate 20-i and 2-morpholinoethan-1-amine (35 mg, 54%). ¹H NMR (400 MHz, DMSO) δ 11.99 (s, 1H), 7.77 (d, J=8.6 Hz, 2H), 7.62-7.55 (m, 3H), 7.53-7.47 (m, 2H), 7.34 (d, J=16.7 Hz, 1H), 7.26 (dd, J=8.6, 1.8 Hz, 1H), 3.59 (t, J=4.6 Hz, 4H), 3.52-3.41 (m, 2H), 2.55 (t, J=6.6 Hz, 2H), 2.46 (t, J=4.5 Hz, 4H). Mass calculated for (C₂₃H₂₃BrClN₃O₂−H)⁻ 486.1, found 485.9.

### Compound 30: (E)-6-Bromo-2-(4-chlorostyryl)-N-(2-(4-hydroxypiperidin-1-yl)ethyl)-1 H-indole-3-carboxamide

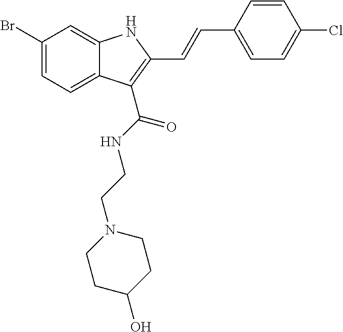 [SMILES: O=C(NCCN1CCC(O)CC1)c1c(/C=C/c2ccc(Cl)cc2)[nH]c2cc(Br)ccc12]

Prepared according to general method XIV from intermediate 20-i and 1-(2-aminoethyl)piperidin-4-ol (44 mg, 66%). ¹H NMR (400 MHz, MeOD) δ 7.80-7.71 (m, 2H), 7.60-7.55 (m, 3H), 7.40 (d, 2H), 7.30-7.21 (m, 2H), 3.84-3.75 (m, 1H), 3.70 (t, J=6.4 Hz, 2H), 3.22-3.13 (m, 2H), 2.94 (t, J=6.4 Hz, 2H), 2.67 (s, 2H), 2.05-1.94 (m, 2H), 1.77-1.65 (m, 2H). Mass calculated for (C₂₄H₂₅BrClN₃O₂−H)⁻ 500.1, found 499.9.

### Compound 31: (E)-6-Bromo-2-(4-chlorostyryl)-N-(2-(4-methylpiperazin-1-yl)ethyl)-1 H-indole-3-carboxamide

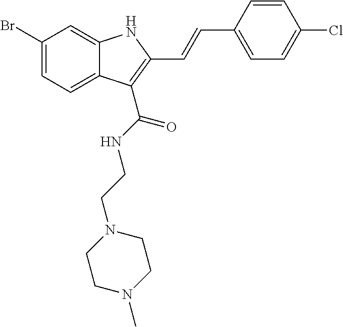 [SMILES: CN1CCN(CCNC(=O)c2c(/C=C/c3ccc(Cl)cc3)[nH]c3cc(Br)ccc23)CC1]

Prepared according to general method XIV from intermediate 20-i and 2-(4-methylpiperazin-1-yl)ethan-1-amine (43 mg, 64%). ¹H NMR (400 MHz, MeOD) δ 7.78-7.69 (m, 2H), 7.60-7.52 (m, 3H), 7.39 (dd, J=8.7, 2.4 Hz, 2H), 7.29-7.18 (m, 2H), 3.62 (t, J=6.4 Hz, 2H), 2.85-2.47 (m, 10H), 2.36 (s, 3H). Mass calculated for (C₂₄H₂₆BrClN₄O−H)⁻ 499.1, found 499.0.

### Compound 32: (E)-6-Bromo-2-(4-chlorostyryl)-N-(1,3-dihydroxypropan-2-yl)-1H-indole-3-carboxamide

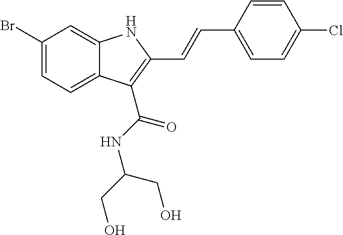 [SMILES: O=C(NC(CO)CO)c1c(/C=C/c2ccc(Cl)cc2)[nH]c2cc(Br)ccc12]

Prepared according to general method XIV from intermediate 20-i and 2-aminopropane-1,3-diol (29 mg, 46%). ¹H NMR (400 MHz, DMSO) δ 12.07 (s, 1H), 7.78 (d, J=16.7 Hz, 1H), 7.69 (d, J=8.5 Hz, 1H), 7.63-7.58 (m, 2H), 7.57 (d, J=1.8 Hz, 1H), 7.52-7.46 (m, 2H), 7.44 (d, J=8.1 Hz, 1H), 7.36 (d, J=16.7 Hz, 1H), 7.24 (dd, J=8.5, 1.8 Hz, 1H), 4.78 (s, 2H), 4.05 (dt, J=8.1, 5.8 Hz, 1H), 3.60 (d, J=5.0 Hz, 4H. Mass calculated for (C₂₀H₁₈BrClN₂O₃−H)⁻ 447.0, found 446.8.

### Compound 33: (E)-tert-Butyl 4-(2-(6-bromo-2-(4-chlorostyryl)-1H-indole-3-carboxamido)ethyl)piperazine-1-carboxylate

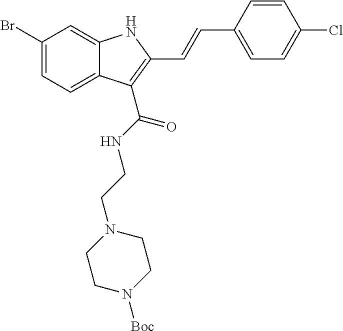 [SMILES: CC(C)(C)OC(=O)N1CCN(CCNC(=O)c2c(/C=C/c3ccc(Cl)cc3)[nH]c3cc(Br)ccc23)CC1]

Prepared according to general method XIV from intermediate 20-i and tert-butyl 4-(2-aminoethyl)piperazine-1-carboxylate (298 mg, 75%). ¹H NMR (400 MHz, MeOD) δ 7.78 (d, J=5.5 Hz, 1H), 7.75 (d, J=2.6 Hz, 1H), 7.63-7.57 (m, 3H), 7.44-7.40 (m, 2H), 7.31-7.24 (m, 2H), 3.64 (t, J=6.4 Hz, 2H), 3.45 (t, J=5.2 Hz, 5H), 2.71 (t, J=6.4 Hz, 2H), 2.55 (t, J=5.0 Hz, 4H), 1.48 (s, 9H). Mass calculated for (C₂₈H₃₂BrClN₄O₃−H)⁻ 585.1, found 584.9.

### Compound 34: (E)-6-Bromo-2-(3,5-dichlorostyryl)-N-(2-(dimethylamino)ethyl)-1H-indole-3-carboxamide

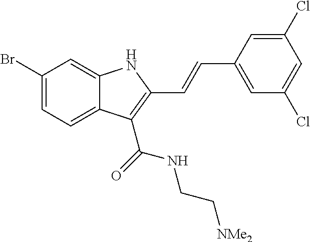 [SMILES: CN(C)CCNC(=O)c1c(/C=C/c2cc(Cl)cc(Cl)c2)[nH]c2cc(Br)ccc12]

Prepared according to general method XIII from intermediate 34-ii and N¹,N¹-dimethylethane-1,2-diamine (15 mg, 26%). ¹H NMR (400 MHz, DMSO) δ 12.02 (s, 1H), 7.88 (t, J=5.5 Hz, 1H), 7.78 (d, J=16.6 Hz, 1H), 7.72 (d, J=8.6 Hz, 1H), 7.58 (s, 4H), 7.31-7.23 (m, 2H), 3.44 (q, J=6.2 Hz, 2H), 2.60-2.53 (m, 2H), 2.31 (s, 6H). Mass calculated for (C₂₁H₂₀BrCl₂N₃O+H)⁺482.0, found 481.8.

### Compound 35: (E)-6-Bromo-N-(2-hydroxyethyl)-2-(2-(5-methoxypyridin-2-yl)vinyl)-1 H-indole-3-carboxamide

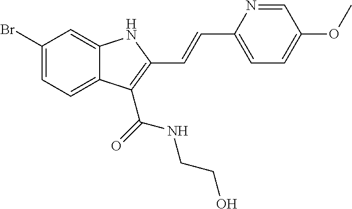 [SMILES: COc1ccc(/C=C/c2[nH]c3cc(Br)ccc3c2C(=O)NCCO)nc1]

Prepared according to general method XIV from intermediate 35-iv and 2-aminoethanol (36 mg, 86%). ¹H NMR (400 MHz, DMSO) δ 11.99 (s, 1H), 8.36 (d, J=2.9 Hz, 1H), 7.99 (d, J=16.5 Hz, 1H), 7.81 (t, J=5.5 Hz, 1H), 7.70 (d, J=8.6 Hz, 1H), 7.55 (d, J=1.7 Hz, 1H), 7.52 (d, J=8.6 Hz, 1H), 7.43 (dd, J=8.7, 3.0 Hz, 1H), 7.36 (d, J=16.4 Hz, 1H), 7.24 (dd, J=8.5, 1.8 Hz, 1H), 4.77 (t, J=5.5 Hz, 1H), 3.58 (q, J=6.1 Hz, 2H), 3.40 (q, J=6.0 Hz, 2H). Mass calculated for (C₁₉H₁₈BrN₃O₃+H)⁺416.1, found 416.1.

### Compound 36: (E)-6-bromo-2-(2-(6-chloropyridin-3-yl)vinyl)-N,N-dimethyl-1H-indole-3-carboxamide

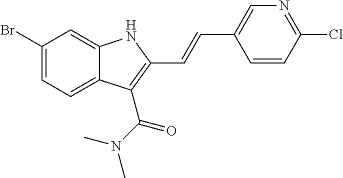 [SMILES: CN(C)C(=O)c1c(/C=C/c2ccc(Cl)nc2)[nH]c2cc(Br)ccc12]

Prepared according to general method XIII from intermediate 36-iii and dimethylamine (38 mg, 72%). ¹H NMR (600 MHz, DMSO) δ 11.99 (s, 1H), 8.55 (d, J=2.4 Hz, 1H), 8.13 (dd, J=8.4, 2.5 Hz, 1H), 7.58 (d, J=1.5 Hz, 1H), 7.55 (d, J=8.4 Hz, 1H), 7.39 (d, J=8.5 Hz, 1H), 7.32 (d, J=16.7 Hz, 1H), 7.29 (d, J=16.7 Hz, 1H), 7.22 (dd, J=8.5, 1.7 Hz, 1H), 3.01 (s, 6H). Mass calculated for (C₁₈H₁₅BrClN₃O+H)⁺406.0, found 406.0.

### Compound 37: (E)-6-Bromo-2-(2-(6-chloropyridin-3-yl)vinyl)-N-(pyridin-3-ylmethyl)-1 H-indole-3-carboxamide

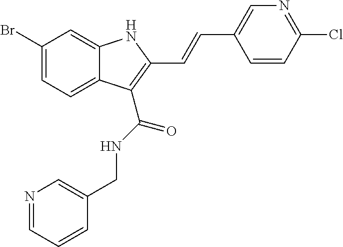 [SMILES: O=C(NCc1cccnc1)c1c(/C=C/c2ccc(Cl)nc2)[nH]c2cc(Br)ccc12]

Prepared according to general method XIII from intermediate 36-iii and pyridin-3-ylmethanamine (27 mg, 73%). ¹H NMR (600 MHz, DMSO) δ 12.11 (s, 1H), 8.63 (s, 1H), 8.55 (t, J=5.9 Hz, 1H), 8.53 (d, J=2.4 Hz, 1H), 8.48 (d, J=3.7 Hz, 1H), 8.05 (dd, J=8.4, 2.5 Hz, 1H), 7.84-7.76 (m, 2H), 7.75 (d, J=8.6 Hz, 1H), 7.61-7.56 (m, 2H), 7.40 (dd, J=7.8, 4.7 Hz, 1H), 7.34 (d, J=16.7 Hz, 1H), 7.27 (dd, J=8.6, 1.7 Hz, 1H), 4.56 (d, J=5.9 Hz, 2H). Mass calculated for (C₂₂H₁₆BrClN₄O+H)⁺469.0, found 468.9.

### Compound 38: (E)-2-(6-Bromo-2-(4-chlorostyryl)-1H-indole-3-carboxamido)acetic Acid

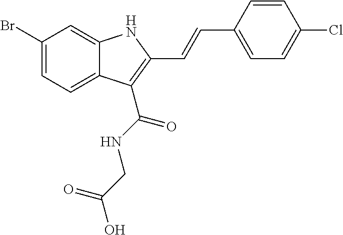 [SMILES: O=C(O)CNC(=O)c1c(/C=C/c2ccc(Cl)cc2)[nH]c2cc(Br)ccc12]

Prepared according to general method XIV from intermediate 20-i and glycine (24 mg, 42%). ¹H NMR (400 MHz, DMSO) δ 12.07 (s, 1H), 8.18 (t, J=5.6 Hz, 1H), 7.85 (d, J=16.7 Hz, 1H), 7.77 (d, J=8.6 Hz, 1H), 7.61 (d, J=8.5 Hz, 2H), 7.58 (d, J=1.2 Hz, 1H), 7.49 (d, J=8.4 Hz, 2H), 7.36 (d, J=16.7 Hz, 1H), 7.26 (dd, J=8.6, 1.4 Hz, 1H), 4.00 (d, J=5.8 Hz, 2H). Mass calculated for (C₁₉H₁₄BrClN₂O₃−H)⁻ 430.0, found 432.8.

### Compound 39: (E)-3-(6-Bromo-2-(4-chlorostyryl)-1H-indole-3-carboxamido)propanoic Acid

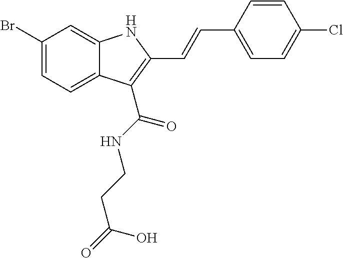 [SMILES: O=C(O)CCNC(=O)c1c(/C=C/c2ccc(Cl)cc2)[nH]c2cc(Br)ccc12]

Prepared according to general method XIV from intermediate 20-i and 3-aminopropanoic acid

(22 mg, 37%). ¹H NMR (400 MHz, DMSO) δ 12.31 (s, 1H), 12.00 (s, 1H), 8.00 (t, J=5.4 Hz, 1H), 7.72 (d, J=11.1 Hz, 1H), 7.69 (d, J=2.9 Hz, 1H), 7.61 (d, J=8.5 Hz, 2H), 7.55 (d, J=1.6 Hz, 1H), 7.50 (d, J=8.5 Hz, 2H), 7.33 (d, J=16.7 Hz, 1H), 7.23 (dd, J=8.6, 1.7 Hz, 1H), 3.59-3.47 (m, 2H), 2.59 (t, J=6.9 Hz, 2H). Mass calculated for (C₂₀H₁₆BrClN₂O₃−H)⁻ 447.0, found 446.8.

### Compound 40: (E)-6-Bromo-2-(4-chlorostyryl)-N-(2-(1-methylpiperidin-4-yl)ethyl)-1 H-indole-3-carboxamide

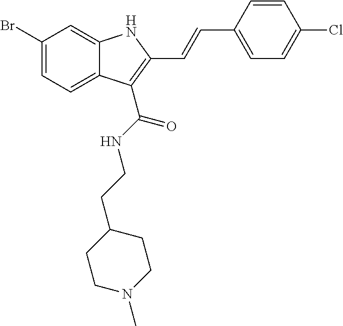 [SMILES: CN1CCC(CCNC(=O)c2c(/C=C/c3ccc(Cl)cc3)[nH]c3cc(Br)ccc23)CC1]

Prepared according to general method XIV from intermediate 20-i and 2-(1-methylpiperidin-4-yl)ethan-1-amine (40 mg, 62%). ¹H NMR (400 MHz, DMSO-d₆) δ 11.99 (s, 1H), 7.93 (t, J=5.7 Hz, 1H), 7.70 (d, J=11.0 Hz, 1H), 7.67 (d, J=2.8 Hz, 1H), 7.61-7.54 (m, 3H), 7.50 (d, J=8.5 Hz, 2H), 7.34 (d, J=16.7 Hz, 1H), 7.25 (dd, J=8.5, 1.8 Hz, 1H), 3.38 (s, 2H), 2.91-2.80 (m, 2H), 2.24 (s, 3H), 2.01 (bs, 2H), 1.84-1.66 (m, 2H), 1.57-1.44 (m, 2H), 1.37 (bs, 1H), 1.30-1.16 (m, 2H). Mass calculated for (C₂₅H₂₇BrClN₃O+H)⁺502.1, found 502.0.

### Compound 41: (E)-6-Bromo-2-(4-chlorostyryl)-N-(2-(4-hydroxycyclohexyl)ethyl)-1H-indole-3-carboxamide (Mixture of Diasteriomers)

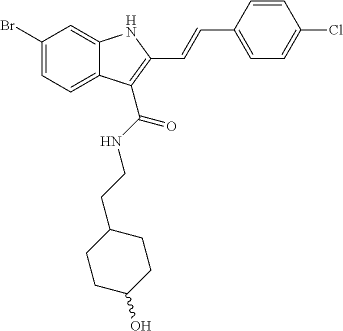 [SMILES: O=C(NCCC1CCC(O)CC1)c1c(/C=C/c2ccc(Cl)cc2)[nH]c2cc(Br)ccc12]

Prepared according to general method XIV from intermediate 20-i and 4-(2-aminoethyl)cyclohexan-1-ol (28 mg, 42%). ¹H NMR (400 MHz, DMSO-d₆) δ 11.98 (s, 2H), 7.96-7.87 (m, 2H), 7.74-7.64 (m, 4H), 7.60-7.54 (m, 6H), 7.53-7.47 (m, 4H), 7.33 (d, J=16.7 Hz, 2H), 7.27-7.21 (m, 2H), 4.48 (d, J=4.4 Hz, 1H), 4.27 (d, J=3.4 Hz, 1H), 3.74 (s, 1H), 3.31 (s, 1H), 1.87-1.71 (m, 4H), 1.63-1.21 (m, 11H), 1.13 (q, J=11.8 Hz, 2H), 1.04-0.87 (m, 2H). Mass calculated for (C₁₉H₁₃BrClN₃O−H)⁻ 501.1, found 501.1.

### Compound 42: (E)-6-Bromo-2-(4-chlorostyryl)-N-(2-(tetrahydro-2H-pyran-4-yl)ethyl)-1H-indole-3-carboxamide

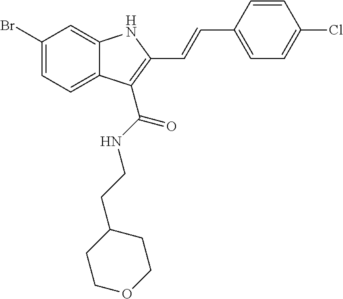 [SMILES: O=C(NCCC1CCOCC1)c1c(/C=C/c2ccc(Cl)cc2)[nH]c2cc(Br)ccc12]

Prepared according to general method XIV from intermediate 20-i and 2-(tetrahydro-2H-pyran-4-yl)ethan-1-amine (53 mg, 82%). ¹H NMR (400 MHz, DMSO-d₆) δ 11.98 (s, 1H), 7.94 (t, J=5.6 Hz, 1H), 7.73-7.65 (m, 2H), 7.61-7.56 (m, 2H), 7.56 (d, J=1.8 Hz, 1H), 7.52-7.47 (m, 2H), 7.33 (d, J=16.7 Hz, 1H), 7.25 (dd, J=8.6, 1.8 Hz, 1H), 3.89-3.80 (m, 2H), 3.43-3.34 (m, 2H), 3.31-3.21 (m, 2H), 1.74-1.47 (m, 5H), 1.29-1.11 (m, 2H). Mass calculated for (C₂₄H₂₄BrClN₂O₂−H)⁻ 487.1, found 487.0.

### Compound 43: (E)-6-Bromo-2-(4-chlorostyryl)-N-(3-(4-methylpiperazin-1-yl)propyl)-1 H-indole-3-carboxamide

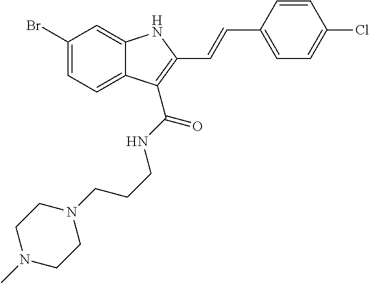 [SMILES: CN1CCN(CCCNC(=O)c2c(/C=C/c3ccc(Cl)cc3)[nH]c3cc(Br)ccc23)CC1]

Prepared according to general method XIV from intermediate 20-i and 3-(4-methylpiperazin-1-yl)propan-1-amine (120 mg, 88%). ¹H NMR (400 MHz, DMSO-d₆) δ 11.99 (s, 1H), 7.97 (t, J=5.6 Hz, 1H), 7.75-7.66 (m, 2H), 7.58 (d, J=8.6 Hz, 2H), 7.56 (d, J=1.8 Hz, 1H), 7.49 (d, J=8.6 Hz, 2H), 7.33 (d, J=16.7 Hz, 1H), 7.25 (dd, J=8.5, 1.8 Hz, 1H), 3.37-3.32 (m, 2H), 2.46-2.36 (m, 12H), 2.19 (s, 3H), 1.82-1.67 (m, 2H). Mass calculated for (C₂₅H₂₈BrClN₄O+H)⁺517.1, found 517.1.

### Compound 44: (E)-6-Bromo-2-(4-chlorostyryl)-N-(4-(4-methylpiperazin-1-yl)butyl)-1 H-indole-3-carboxamide

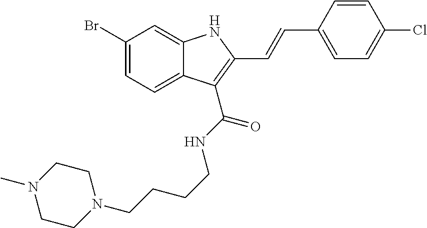 [SMILES: CN1CCN(CCCCNC(=O)c2c(/C=C/c3ccc(Cl)cc3)[nH]c3cc(Br)ccc23)CC1]

Prepared according to general method XIV from intermediate 20-i and 4-(4-methylpiperazin-1-yl)butan-1-amine (125 mg, 89%). ¹H NMR (400 MHz, DMSO-d₆) δ 12.29 (s, 1H), 7.98 (t, J=5.7 Hz, 1H), 7.74-7.63 (m, 2H), 7.61-7.54 (m, 3H), 7.53-7.39 (m, 3H), 7.23 (dd, J=8.5, 1.8 Hz, 1H), 3.32-3.22 (m, 4H), 2.40-2.17 (m, 8H), 2.11 (s, 3H), 1.64-1.46 (m, 4H). Mass calculated for (C₂₆H₃₀BrClN₄O+H)⁺529.1, found 529.2.

### Compound 45: (E)-6-Bromo-2-(4-chlorostyryl)-N-(4-morpholinobutyl)-1H-indole-3-carboxamide

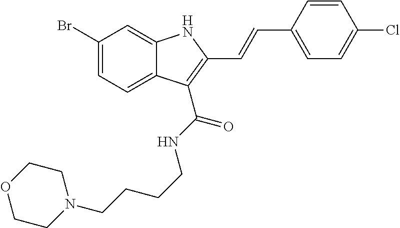 [SMILES: O=C(NCCCCN1CCOCC1)c1c(/C=C/c2ccc(Cl)cc2)[nH]c2cc(Br)ccc12]

Prepared according to general method XIV from intermediate 20-i and 4-morpholinobutan-1-amine (15 mg, 77%). ¹H NMR (400 MHz, DMSO-d₆) δ 12.00 (s, 1H), 7.97 (t, J=5.7 Hz, 1H), 7.74-7.64 (m, 2H), 7.60-7.53 (m, 3H), 7.50 (d, J=8.5 Hz, 2H), 7.33 (d, J=16.7 Hz, 1H), 7.24 (dd, J=8.5, 1.8 Hz, 1H), 3.54 (t, J=4.6 Hz, 4H), 3.33-3.23 (m, 2H), 2.38-2.27 (m, 6H), 1.66-1.41 (m, 4H). Mass calculated for (C₂₅H₂₇BrClN₃O₂+H)⁺518.1, found 518.1.

### Compound 46: 1-(6-Bromo-2-(4-chlorophenethyl)-1H-indol-3-yl)-2,2,2-trifluoroethanone Oxime

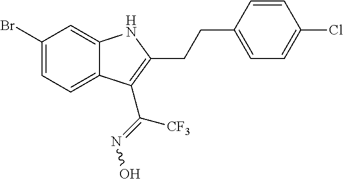 [SMILES: ON=C(c1c(CCc2ccc(Cl)cc2)[nH]c2cc(Br)ccc12)C(F)(F)F]

Prepared according to general method XI from intermediate 17-i (26 mg, 54%). Present as 2:1 mixture of isomers.

Major isomer: ¹H NMR (600 MHz, DMSO) δ 11.74 (s, 1H), 7.57 (s, 1H), 7.32 (d, J=8.3 Hz, 2H), 7.24 (d, J=8.5 Hz, 1H), 7.22-7.16 (m, 3H), 3.03-2.92 (m, 4H).

Minor isomer: ¹H NMR (600 MHz, DMSO) δ 11.79 (s, 1H), 7.57 (s, 1H), 7.34 (d, J=8.2 Hz, 2H), 7.21-7.16 (m, 4H), 2.97-2.92 (m, 2H), 2.89-2.82 (m, 2H).

Mass calculated for (C₁₈H₁₃BrClF₃N₂O−H)⁻ 445.0, found 444.9.

### Compound 47: (6-Bromo-2-(4-chlorophenethyl)-1H-indol-3-yl)(morpholino)methanone

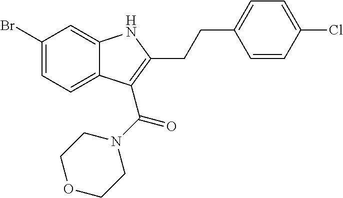 [SMILES: O=C(c1c(CCc2ccc(Cl)cc2)[nH]c2cc(Br)ccc12)N1CCOCC1]

Prepared according to general method XII and XIII from intermediate 17-i (23 mg, 65%). ¹H NMR (500 MHz, CDCl₃) δ 8.33 (s, 1H), 7.40 (d, J=1.4 Hz, 1H), 7.29 (d, J=8.5 Hz, 1H), 7.26-7.20 (m, 4H), 7.02 (d, J=8.4 Hz, 2H), 3.81-3.35 (m, 8H), 3.11 (t, J=7.1 Hz, 2H), 2.96 (t, J=7.4 Hz, 2H). Mass calculated for (C₂₁H₂₀BrClN₂O₂+H)⁺448.0, found 448.9.

### Synthesis of Compound 48

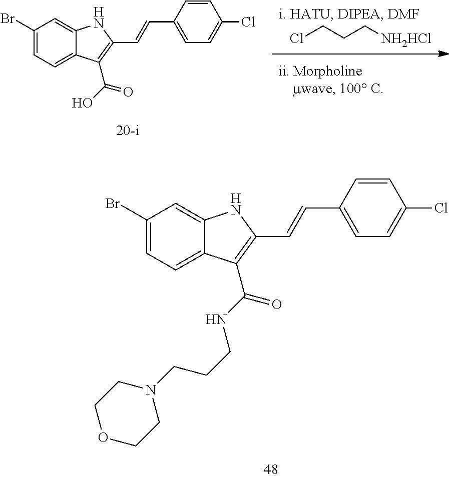

### Compound 48: (E)-6-Bromo-2-(4-chlorostyryl)-N-(3-morpholinopropyl)-1H-indole-3-carboxamide

A solution of 20-i (100 mg, 0.27 mmol), DIPEA (150 uL, 0.86 mmol) and HATU (110 mg, 0.29 mmol) in DMF (3 mL) was stirred at rt for 5 min followed by the addition of 3-chloropropan-1-amine hydrochloride (50 mg, 0.38 mmol). The resulting mixture was stirred at rt for 4 h. Morpholine (0.1 mL, 1.14 mmol) was added and the mixture was heated with microwave at 100° C. for 1 h. The reaction mixture was diluted with EtOAc and washed with H₂O, brine, dried over anhydrous Na₂SO₄, filtered and concentrated under reduced pressure. The crude material was purified by silica gel chromatography, eluting with an EtOAc/hexanes gradient, to provide the compound 48 as a pale brown solid (29 mg, 22%). ¹H NMR (400 MHz, DMSO-d₆) δ 11.97 (s, 1H), 7.97 (t, J=5.7 Hz, 1H), 7.75-7.67 (m, 2H), 7.61-7.54 (m, 3H), 7.53-7.46 (m, 2H), 7.33 (d, J=16.7 Hz, 1H), 7.25 (dd, J=8.5, 1.8 Hz, 1H), 3.55 (t, J=4.7 Hz, 4H), 3.39-3.33 (m, 2H), 2.38 (d, J=8.6 Hz, 6H), 1.75 (p, J=7.0 Hz, 2H). Mass calculated for (C₂₄H₂₅BrClN₃O₂+H)⁺504.1, found 504.0.

### General Method XV

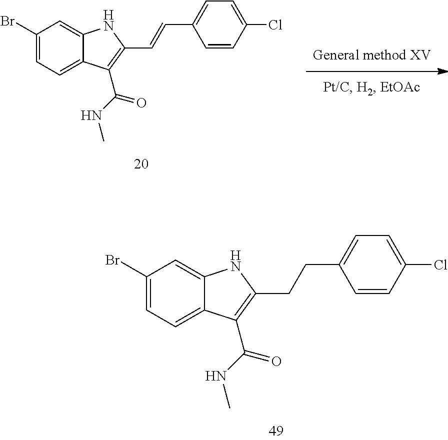 [SMILES: CNC(=O)c1c(/C=C/c2ccc(Cl)cc2)[nH]c2cc(Br)ccc12 ; CNC(=O)c1c(CCc2ccc(Cl)cc2)[nH]c2cc(Br)ccc12]

To a stirred solution of the corresponding amide (1.0 mmol) in EtOAc (10 mL) was added Pt/C (10% on carbon, 100 mg). The mixture was purged with H₂ for 30 min and then stirred under H₂ (1 atm) for 18 h. The reaction mixture was filtered through a pad of celite and concentrated under reduced pressure. The residue was purified by silica gel chromatography, eluting with an EtOAc/hexanes gradient, followed by preparative HLPC (ACN/H₂O with 0.1% formic acid) to provide the desired product.

### Compound 49: 6-Bromo-2-(4-chlorophenethyl)-N-methyl-1H-indole-3-carboxamide

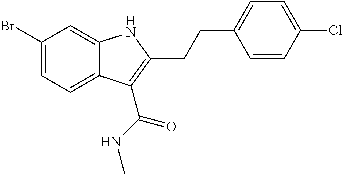 [SMILES: CNC(=O)c1c(CCc2ccc(Cl)cc2)[nH]c2cc(Br)ccc12]

Prepared according to general method XV from compound 20 (6 mg, 36%). ¹H NMR (400 MHz, MeOD) δ 7.60 (d, J=8.6 Hz, 1H), 7.49-7.45 (m, 1H), 7.24-7.19 (m, 3H), 7.13 (d, J=8.5 Hz, 2H), 3.33-3.27 (m, 2H), 3.00 (t, J=7.7 Hz, 2H), 2.91 (s, 3H). Mass calculated for (C₁₈H₁₆BrClN₂O+H)⁺393.0, found 393.0.

### Compound 50: 6-Bromo-2-(4-chlorophenethyl)-N,N-dimethyl-1H-indole-3-carboxamide (50)

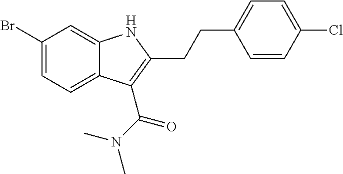 [SMILES: CN(C)C(=O)c1c(CCc2ccc(Cl)cc2)[nH]c2cc(Br)ccc12]

Prepared according to general method XV from compound 21 except MeOH/EtOAc (1/5) mixture was used as solvent instead of EtOAc (8 mg, 27%). ¹H NMR (400 MHz, MeOD) δ 7.50 (dd, J=1.6, 0.6 Hz, 1H), 7.23-7.15 (m, 4H), 7.10 (d, J=8.5 Hz, 2H), 3.16 (t, J=7.1 Hz, 2H), 3.00 (t, J=7.2 Hz, 2H), 2.97 (bs, 6H). Mass calculated for (C₁₉H₁₈BrClN₂O+H)⁺405.0, found 405.0.

### Compound 51: 2-(4-Chlorophenethyl)-N,N-dimethyl-1H-indole-3-carboxamide

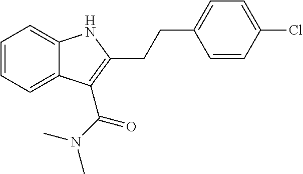 [SMILES: CN(C)C(=O)c1c(CCc2ccc(Cl)cc2)[nH]c2ccccc12]

Prepared according to general method XV from compound 21 except MeOH/EtOAc (1/5) mixture was used as solvent instead of EtOAc (5 mg, 21%). ¹H NMR (400 MHz, MeOD) δ 7.36-7.32 (m, 1H), 7.30-7.26 (m, 1H), 7.20 (d, J=8.4 Hz, 2H), 7.13-7.03 (m, 4H), 3.18 (t, J=7.1 Hz, 2H), 3.04 (bs, 6H), 3.01 (t, J=7.3 Hz, 2H). Mass calculated for (C₁₉H₁₉ClN₂O+H)⁺327.1, found 327.2.

### General Method XVI

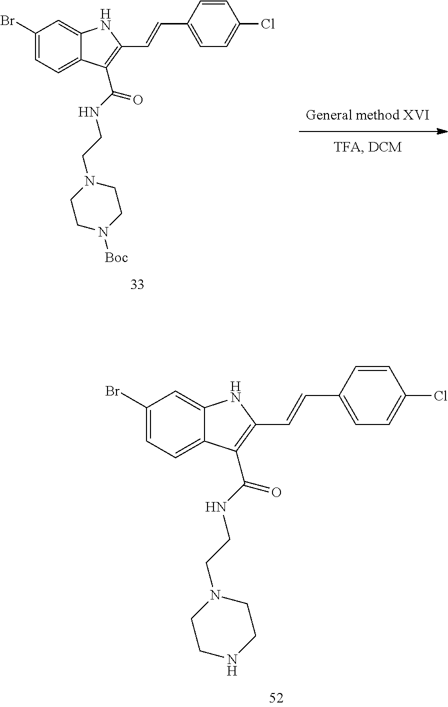 [SMILES: CC(C)(C)OC(=O)N1CCN(CCNC(=O)c2c(/C=C/c3ccc(Cl)cc3)[nH]c3cc(Br)ccc23)CC1 ; O=C(NCCN1CCNCC1)c1c(/C=C/c2ccc(Cl)cc2)[nH]c2cc(Br)ccc12]

To a stirred solution of N-Boc intermediate (1.0 mmol) in CH₂Cl₂ (12 ml) was added trifluoroacetic acid (6 ml). The resulting solution was stirred at rt for 2 h and concentrated under reduced pressure. The residue was dissolved in EtOAc and washed with saturated aqueous solution of NaHCO₃, H₂O, brine, dried over anhydrous Na₂SO₄, filtered and concentrated under reduced pressure. The crude material was purified by silica gel chromatography, eluting with a MeOH/DCM gradient, to provide the desired product.

### Compound 52: (E)-6-Bromo-2-(4-chlorostyryl)-N-(2-(piperazin-1-yl)ethyl)-1H-indole-3-carboxamide

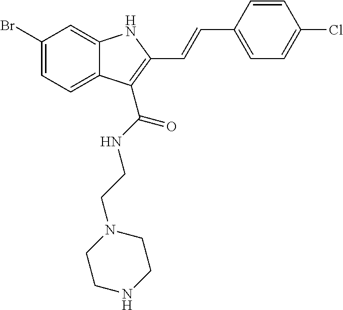 [SMILES: O=C(NCCN1CCNCC1)c1c(/C=C/c2ccc(Cl)cc2)[nH]c2cc(Br)ccc12]

Prepared according to general method XVI from 33 (244 mg, 99%). ¹H NMR (400 MHz, Methanol-d₄) δ 7.74 (dd, J=12.6, 4.1 Hz, 2H), 7.59-7.52 (m, 3H), 7.41-7.36 (m, 2H), 7.28-7.19 (m, 2H), 3.62 (t, J=6.5 Hz, 2H), 2.91 (t, J=4.9 Hz, 4H), 2.68 (t, J=6.5 Hz, 2H), 2.64-2.54 (m, 4H). Mass calculated for (C₂₃H₂₄BrClN₄O−H)⁻ 486.06, found 484.9.

### Compound 53: (E)-6-Bromo-2-(4-chlorostyryl)-N-(piperidin-4-yl)-1H-indole-3-carboxamide

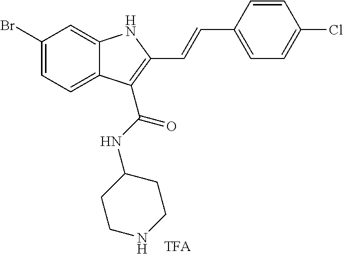 [SMILES: O=C(NC1CCNCC1)c1c(/C=C/c2ccc(Cl)cc2)[nH]c2cc(Br)ccc12 ; "TFA"]

Prepared according to general method XIII and XVI from 20-i and tert-butyl 4-aminopiperidine-1-carboxylate (31 mg, 70%). ¹H NMR (600 MHz, MeOD) δ 8.52 (s, 1H), 7.67 (d, J=16.7 Hz, 1H), 7.65 (d, J=8.6 Hz, 1H), 7.60-7.55 (m, 3H), 7.41 (d, J=8.4 Hz, 2H), 7.30-7.24 (m, 2H), 4.31-4.21 (m, 1H), 3.51 (bd, J=13.0 Hz, 2H), 3.20 (td, J=12.8, 2.8 Hz, 2H), 2.32 (dd, J=14.1, 2.6 Hz, 2H), 1.97-1.86 (m, 2H). Mass calculated for (C₂₄H₂₁BrClF₃N₃O₂+H-TFA)⁺460.0, found 460.0.

### Compound 54: (S,E)-2-Amino-6-(6-bromo-2-(4-chlorostyryl)-1H-indole-3-carboxamido)hexanoic Acid

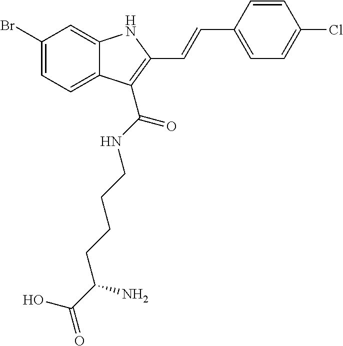 [SMILES: N[C@@H](CCCCNC(=O)c1c(/C=C/c2ccc(Cl)cc2)[nH]c2cc(Br)ccc12)C(=O)O]

Prepared according to general method XIII and XVI from 20-i and (tert-butoxycarbonyl)-L-lysine (52 mg, 78%). ¹H NMR (400 MHz, DMSO) δ 12.03 (s, 1H), 8.16 (bs, 2H), 7.94 (t, J=5.5 Hz, 1H), 7.77-7.66 (m, 2H), 7.62-7.53 (m, 3H), 7.50 (d, J=8.5 Hz, 2H), 7.34 (d, J=16.6 Hz, 1H), 7.25 (dd, J=8.5, 1.5 Hz, 1H), 3.85 (t, J=6.0 Hz, 1H), 3.43-3.31 (m, 2H), 1.95-1.73 (m, 2H), 1.69-1.34 (m, 4H). Mass calculated for (C₂₃H₂₃BrClN₃O₃+H)⁺506.1, found 505.9.

### Compound 55: (E)-N,N′-((4,4′-succinylbis(piperazine-4,1-diyl))bis(ethane-2,1-diyl))bis(6-bromo-2-((E)-4-chlorostyryl)-1H-indole-3-carboxamide)

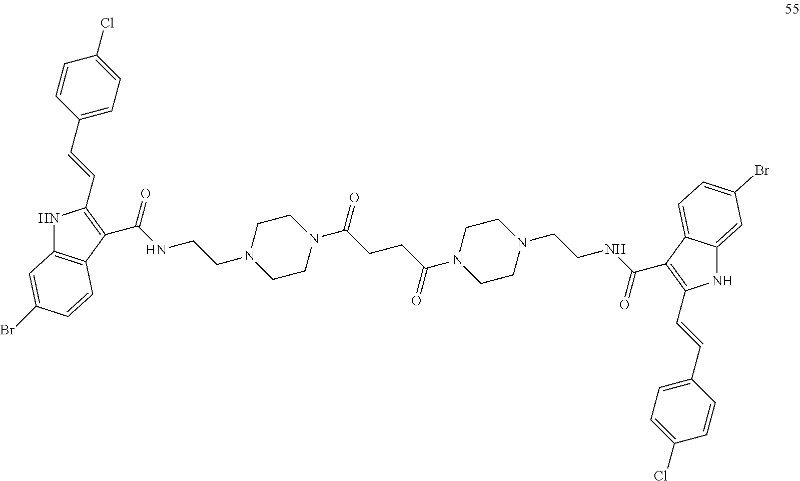 [SMILES: O=C(NCCN1CCN(C(=O)CCC(=O)N2CCN(CCNC(=O)c3c(/C=C/c4ccc(Cl)cc4)[nH]c4cc(Br)ccc34)CC2)CC1)c1c(/C=C/c2ccc(Cl)cc2)[nH]c2cc(Br)ccc12]

Prepared according to general method XIV from compound 52 (2.2 equivalent) and succinic acid (16 mg, 16%). ¹H NMR (400 MHz, DMSO-d₆) δ 11.99 (s, 1H), 7.83-7.71 (m, 3H), 7.62-7.54 (m, 3H), 7.50 (d, J=8.4 Hz, 2H), 7.34 (d, J=16.7 Hz, 1H), 7.27 (dd, J=8.5, 1.8 Hz, 1H), 3.53-3.41 (m, 6H), 2.57 (t, J=6.6 Hz, 1H), 2.54 (s, 2H), 2.42 (s, 2H). Mass calculated for (C₅₀H₅₀Br₂Cl₂N₈O₄+H)⁺ 1055.2, found 1055.0.

### Synthesis of Compound 56

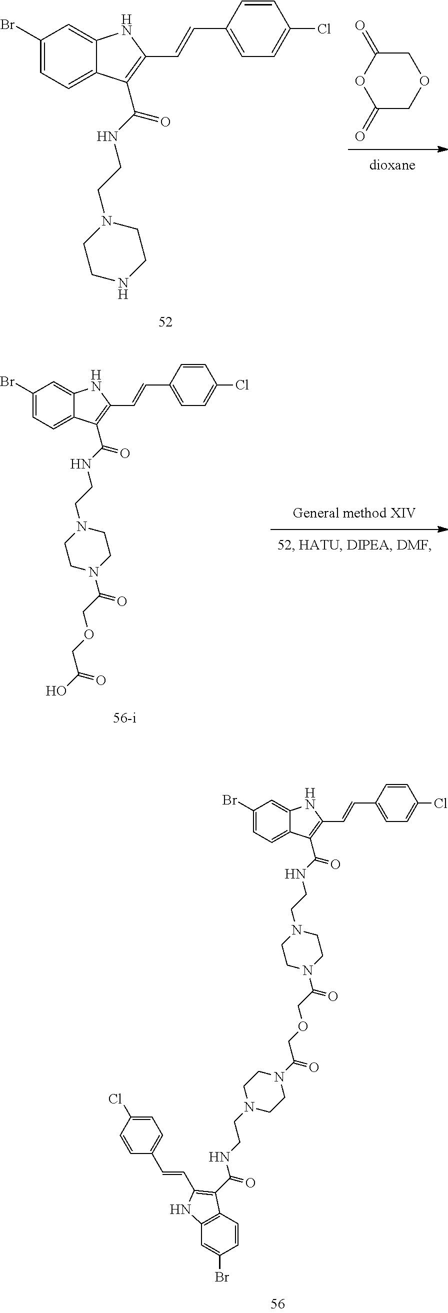 [SMILES: O=C(NCCN1CCNCC1)c1c(/C=C/c2ccc(Cl)cc2)[nH]c2cc(Br)ccc12 ; O=C1COCC(=O)O1 ; O=C(O)COCC(=O)N1CCN(CCNC(=O)c2c(/C=C/c3ccc(Cl)cc3)[nH]c3cc(Br)ccc23)CC1 ; O=C(NCCN1CCN(C(=O)COCC(=O)N2CCN(CCNC(=O)c3c(/C=C/c4ccc(Cl)cc4)[nH]c4cc(Br)ccc34)CC2)CC1)c1c(/C=C/c2ccc(Cl)cc2)[nH]c2cc(Br)ccc12]

### Compound 56: (E)-N,N′-((4,4′-(2,2′-oxybis(acetyl))bis(piperazine-4,1-diyl))bis(ethane-2,1-diyl))bis(6-bromo-2-((E)-4-chlorostyryl)-1H-indole-3-carboxamide)

To a stirred solution of compound 52 (20.3 mg, 0.0416 mmol) in dioxane at 0° C. under Ar was added diglycolic anhydride (5.5 mg, 0.047 mmol). The resulting mixture was stirred for 15 min and then concentrated to give 56-i. Intermediate 56-i was then coupled to another molecule of 52 using general method XIV to give the desired compound 56 (9 mg, 20%). ¹H NMR (400 MHz, DMSO) δ 11.99 (s, 1H), 7.84-7.70 (m, 3H), 7.65-7.54 (m, 2H), 7.50 (d, J=8.4 Hz, 2H), 7.34 (d, J=16.7 Hz, 1H), 7.27 (d, J=8.2 Hz, 1H), 4.22 (s, 2H), 3.45 (bs, 6H), 2.56 (d, J=5.7 Hz, 2H), 2.47 (bs, 4H). Mass calculated for (C₅₀H₅₀Br₂Cl₂N₈O₅+H)⁺1071.2, found 1071.1.

### Synthesis of Compound 57

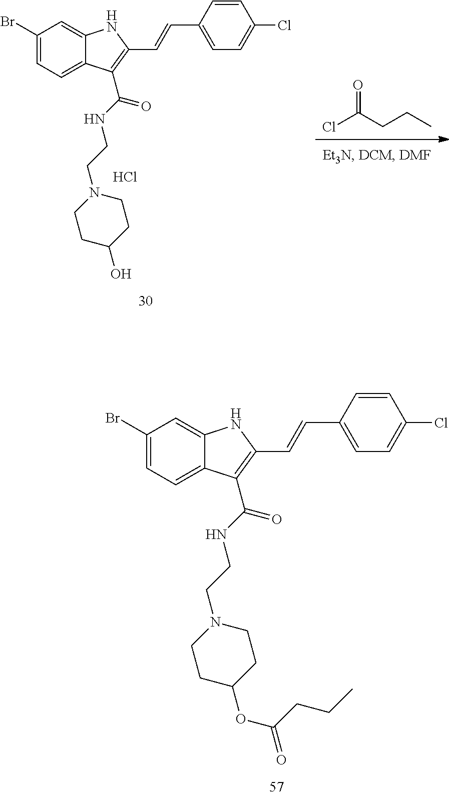

### Compound 57: (E)-1-(2-(6-Bromo-2-(4-chlorostyryl)-1H-indole-3-carboxamido)ethyl)piperidin-4-yl Butyrate

To a stirred solution of compound 30 (50 mg, 93 umol) in DCM (2 mL) and DMF (0.2 mL) was added Et₃N (60 uL, 430 umol) and butyryl chloride (36 uL, 348 umol). The resulting mixture was stirred at rt for 20 h and then diluted with DCM. The mixture was washed with H₂O, brine, dried over anhydrous Na₂SO₄, filtered and concentrated under reduced pressure. The crude product was purified by silica gel chromatography, eluting with an EtOAc/hexanes gradient, to provide compound 57 (34 mg, 53%). ¹H NMR (400 MHz, DMSO-d₆) δ 11.98 (s, 1H), 7.82-7.67 (m, 3H), 7.60 (d, J=8.6 Hz, 2H), 7.56 (d, J=1.8 Hz, 1H), 7.50 (d, J=8.6 Hz, 2H), 7.34 (d, J=16.7 Hz, 1H), 7.25 (dd, J=8.6, 1.8 Hz, 1H), 4.79-4.52 (m, 1H), 3.44 (q, J=6.2 Hz, 2H), 2.85-2.68 (m, 1H), 2.58-2.52 (m, 2H), 2.33-2.22 (m, 4H), 1.90-1.75 (m, 2H), 1.63-1.47 (m, 4H), 0.89 (t, J=7.4 Hz, 3H). Mass calculated for (C₂₃H₂₂BrClN₂O₃+H)⁺574.1, found 574.0.

### Compound 58: (E)-6-Bromo-2-(4-chlorostyryl)-N-(2-(4-(2-hydroxyacetyl)piperazin-1-yl)ethyl)-1H-indole-3-carboxamide

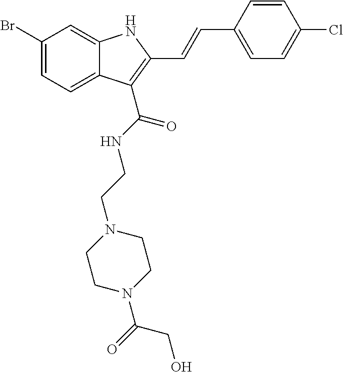 [SMILES: O=C(NCCN1CCN(C(=O)CO)CC1)c1c(/C=C/c2ccc(Cl)cc2)[nH]c2cc(Br)ccc12]

Prepared according to general method XIV from 52 and glycolic acid (46 mg, quantitative). ¹H NMR (400 MHz, DMSO) δ 12.02 (s, 1H), 7.81 (t, J=5.3 Hz, 1H), 7.79-7.69 (m, 2H), 7.61-7.54 (m, 3H), 7.50 (d, J=8.4 Hz, 2H), 7.34 (d, J=16.7 Hz, 1H), 7.27 (dd, J=8.6, 1.3 Hz, 1H), 4.57 (t, J=4.8 Hz, 1H), 4.09 (d, J=5.4 Hz, 2H), 3.52-3.41 (m, 4H), 3.35-3.28 (m, 2H), 2.57 (t, J=6.4 Hz, 2H), 2.49-2.40 (m, 4H). Mass calculated for (C₂₅H₂₆BrClN₄O₃+H)⁺547.1, found 546.9.

### Synthesis of Compound 59

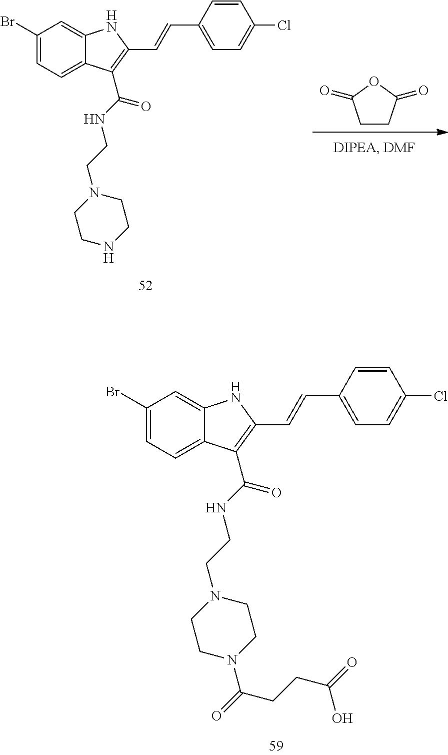 [SMILES: O=C(NCCN1CCNCC1)c1c(/C=C/c2ccc(Cl)cc2)[nH]c2cc(Br)ccc12 ; O=C1CCC(=O)O1 ; O=C(O)CCC(=O)N1CCN(CCNC(=O)c2c(/C=C/c3ccc(Cl)cc3)[nH]c3cc(Br)ccc23)CC1]

### Compound 59: (E)-4-(4-(2-(6-Bromo-2-(4-chlorostyryl)-1H-indole-3-carboxamido)ethyl)piperazin-1-yl)-4-oxobutanoic Acid

A mixture of compound 52 (45 mg, 92 umol), succinic anhydride (15 mg, 150 umol) and DIPEA (50 uL, 287 umol) in DMF (2 mL) was stirred at rt for 90 min. The mixture was purified by preparative HPLC (ACN/H₂O in 0.1% formic acid) followed by silica gel chromatography, eluting with aMeOH/DCM gradient, to provide the compound 59 (18 mg, 33%). ¹H NMR (400 MHz, DMSO) δ 12.00 (s, 1H), 7.83-7.70 (m, 3H), 7.62-7.54 (m, 3H), 7.50 (d, J=8.5 Hz, 2H), 7.34 (d, J=16.5 Hz, 1H), 7.27 (dd, J=8.5, 1.7 Hz, 1H), 3.59 (s, 4H), 3.45 (bs, 6H), 2.65-2.54 (m, 4H), 2.42 (bs, 2H). Mass calculated for (C₂₇H₂₈BrClN₄O₄+H)⁺589.1, found 588.9.

### General Method XVII and XVIII

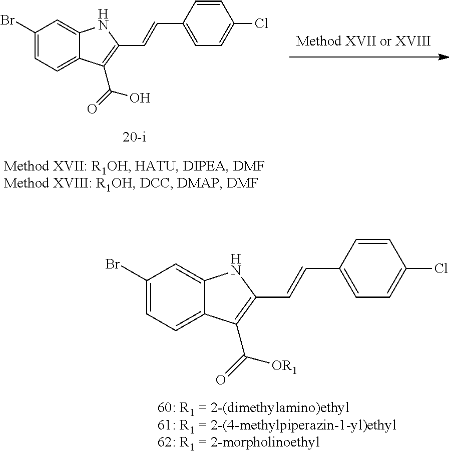

### General Method XVII

A mixture of 20-i (1.0 mmol), DIPEA (4.0 mmol), HATU (1.5 mmol) and the corresponding alcohol (3.0 mmol) in DMF (15 mL) was heated at 50-60° C. for 20 h and then diluted with EtOAc. The organic layer was washed with H₂O, brine, dried over anhydrous Na₂SO₄, filtered and concentrated under reduced pressure. The crude product was purified by silica gel chromatography, eluting with an EtOAc/hexanes or MeOH/DCM gradient, to provide the desired ester.

### General Method XVIII

To a stirred solution of 20-i (1.0 mmol), DMAP (0.1 mmol) and the corresponding alcohol (4.0) in DMF (10 mL) at 0° C. was added DCC (1.2 mmol). The resulting mixture was stirred at 0° C. for 5 min and then at rt for 20 h. The organic layer was washed with H₂O, brine, dried over anhydrous Na₂SO₄, filtered and concentrated under reduced pressure. The crude product was purified by silica gel chromatography, eluting with an EtOAc/hexanes or MeOH/DCM gradient, to provide the desired ester.

### Compound 60: (E)-2-(Dimethylamino)ethyl 6-bromo-2-(4-chlorostyryl)-1H-indole-3-carboxylate

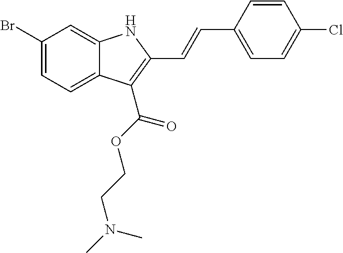 [SMILES: CN(C)CCOC(=O)c1c(/C=C/c2ccc(Cl)cc2)[nH]c2cc(Br)ccc12]

Prepared according to general method XVII from 20-i and 2-(dimethylamino)ethan-1-ol (280 mg, 45%). ¹H NMR (400 MHz, DMSO) δ 12.36 (s, 1H), 8.08 (d, J=16.8 Hz, 1H), 7.97 (d, J=8.6 Hz, 1H), 7.66 (d, J=8.5 Hz, 2H), 7.59 (d, J=1.5 Hz, 1H), 7.54 (d, J=8.5 Hz, 2H), 7.48 (d, J=16.8 Hz, 1H), 7.32 (dd, J=8.6, 1.6 Hz, 1H), 4.39 (t, J=5.6 Hz, 2H), 2.68 (t, J=5.6 Hz, 2H), 2.27 (s, 6H). Mass calculated for (C₂₁H₂₀BrClN₂O₂+H)⁺449.0, found 448.9.

### Compound 61: 2-(4-Methylpiperazin-1-yl)ethyl (E)-6-bromo-2-(4-chlorostyryl)-1H-indole-3-carboxylate

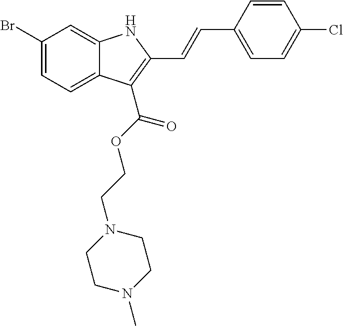 [SMILES: CN1CCN(CCOC(=O)c2c(/C=C/c3ccc(Cl)cc3)[nH]c3cc(Br)ccc23)CC1]

Prepared according to general method XVII from 20-i and 2-(4-methylpiperazin-1-yl)ethan-1-ol (23 mg, 34%). ¹H NMR (400 MHz, DMSO) δ 12.38 (s, 1H), 8.06-7.98 (m, 2H), 7.63 (d, J=8.5 Hz, 2H), 7.60 (d, J=1.6 Hz, 1H), 7.53 (d, J=8.6 Hz, 2H), 7.49 (d, J=16.8 Hz, 1H), 7.32 (dd, J=8.6, 1.7 Hz, 1H), 4.41 (t, J=5.7 Hz, 2H), 2.78 (t, J=5.4 Hz, 2H), 2.55 (bs, 8H), 2.31 (s, 3H). Mass calculated for (C₂₄H₂₅BrClN₃O₂+H)⁺504.1, found 503.9.

### Compound 62: 2-Morpholinoethyl (E)-6-bromo-2-(4-chlorostyryl)-1H-indole-3-carboxylate

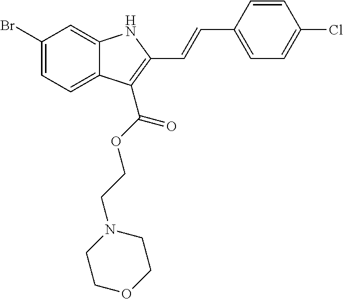 [SMILES: O=C(OCCN1CCOCC1)c1c(/C=C/c2ccc(Cl)cc2)[nH]c2cc(Br)ccc12]

Prepared according to general method XVIII from 20-i and 2-morpholinoethan-1-ol (10 mg, 15%). ¹H NMR (400 MHz, DMSO-d₆) δ 12.49 (s, 1H), 8.06-7.97 (m, 2H), 7.67-7.58 (m, 3H), 7.57-7.47 (m, 3H), 7.32 (dd, J=8.6, 1.8 Hz, 1H), 4.42 (t, J=5.7 Hz, 2H), 3.58 (t, J=4.6 Hz, 4H), 2.50 (bs, 4H). Mass calculated for (C₂₃H₂₂BrClN₂O₃+H)⁺491.1, found 490.9.

### General Method XIX

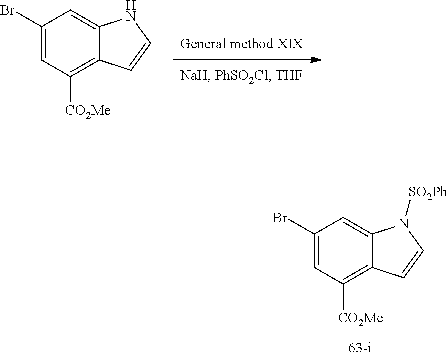

To a stirred solution of the corresponding indole (1.0 mmol) in THF (25 ml) at 0° C. was added NaH (60% in oil, 1.5 mmol) gradually. After stirring at rt for 10 min benzenesulphonyl chloride (1.2 mmol) was added and the mixture was further stirred for 2 h. The reaction was quenched with H₂O and extracted with EtOAc (2×50 ml).

The combined organic layer was washed with brine, dried over anhydrous Na₂SO₄, filtered and concentrated under reduced pressure. The crude product was purified by silica gel chromatography, eluting with an EtOAc/hexanes gradient, to provide the desired product.

### Intermediate 63-i: Methyl 6-bromo-1-(phenylsulfonyl)-1H-indole-4-carboxylate

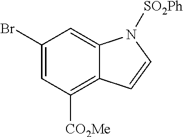

Prepared according to general method XIX from methyl 6-bromo-1H-indole-4-carboxylate (220 mg, 56%). ¹H NMR (400 MHz, CDCl₃) δ 8.41 (dd, J=1.7, 0.8 Hz, 1H), 8.12 (d, J=1.7 Hz, 1H), 7.93-7.87 (m, 2H), 7.69 (d, J=3.7 Hz, 1H), 7.65-7.58 (m, 1H), 7.54-7.47 (m, 2H), 7.36 (dd, J=3.7, 0.8 Hz, 1H), 3.97 (s, 3H).

### General Method XX

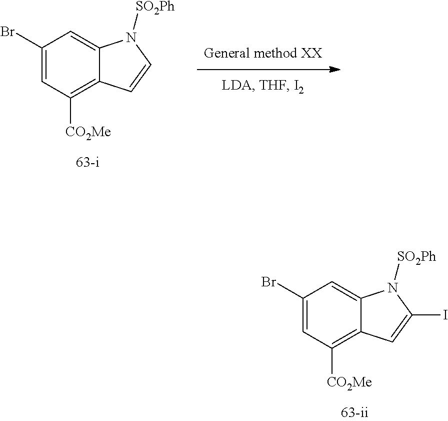

To a stirred solution of protected indole (1 mmol) in anhydrous THF (15 mL), at −78 OC, was added a solution of LDA (1.5 mmol) in THF (5 mL) slowly. The mixture was stirred at −78° C. for 10 min and then warmed to −10 OC for 5 min (except with 4-methyl carboxylate derivative where I₂ was added immediately after the addition of LDA). The solution was re-cooled to −78° C. and then a solution of I₂ (1.5 mmol) in THF (5 mL) was added. The reaction mixture was stirred at 0° C. for 15 minutes and then allowed for warm to rt for 1 h. The reaction was quenched with saturated aqueous NH₄Cl solution and extracted with EtOAc (2×50 ml). The combined organic phases were washed with brine, dried over anhydrous Na₂SO₄, filtered and concentrated in vacuo. The residue was purified by silica gel chromatography eluting with an EtOAc/hexanes gradient to provide the desired intermediate.

### Intermediate 63-iii: Methyl 6-bromo-2-iodo-1H-indole-4-carboxylate

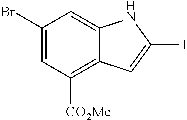 [SMILES: COC(=O)c1cc(Br)cc2[nH]c(I)cc12]

Prepared according to general method XX to give 63-ii from 63-i. Intermediate 63-ii was treated with TBAF as described in general method III to give 63-iii. (16 mg, 32%). ¹H NMR (400 MHz, CDCl₃) δ 8.31 (s, 1H), 8.00 (d, J=1.7 Hz, 1H), 7.69 (dd, J=1.7, 0.9 Hz, 1H), 7.38 (dd, J=2.2, 0.9 Hz, 1H), 4.01 (s, 3H).

### General Method XXI

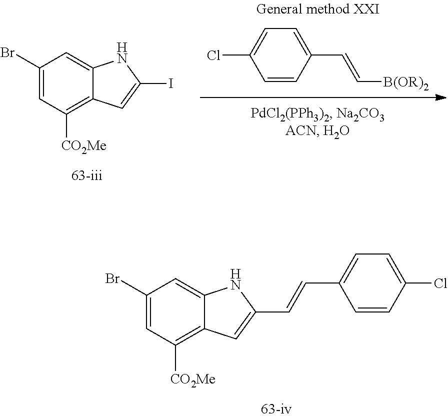

A solution of either pinacol boronate or boronic acid (1 mmol), 2-iodoindole derivative (1 mmol), Na₂CO₃ (1M aqueous solution, 3.5 mmol) in ACN (5 mL) was purged with argon for 10 min followed by the addition of Pd(PPh₃)₂Cl₂ catalyst (10 mol %). The mixture was heated in a sealed tube with microwave at 110° C. for 90 min. The reaction mixture was partitioned between EtOAc (100 mL) and H₂O (50 mL). The organic phase was washed with brine (50 mL), dried over anhydrous Na₂SO₄, filtered and concentrated under reduced pressure. The crude product was purified by silica gel chromatography eluting with either EtOAc/hexanes or MeOH/DCM gradient, to provide the desired adduct.

### Intermediate 63-iv: (E)-Methyl 6-bromo-2-(4-chlorostyryl)-1H-indole-4-carboxylate

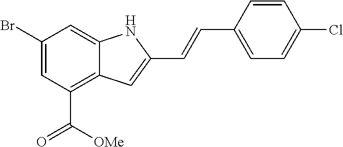 [SMILES: COC(=O)c1cc(Br)cc2[nH]c(/C=C/c3ccc(Cl)cc3)cc12]

Prepared according to general method XXI from 63-iii and (E)-2-(4-chlorostyryl)-4,4,5,5-tetramethyl-1,3,2-dioxaborolane (233 mg, 68%). ¹H NMR (600 MHz, DMSO) δ 11.93 (s, 1H), 7.79-7.76 (m, 2H), 7.64 (d, J=8.5 Hz, 2H), 7.48 (d, J=8.4 Hz, 2H), 7.35 (dd, J=46.9, 16.5 Hz, 2H), 7.14 (d, J=1.3 Hz, 1H), 3.93 (s, 3H). ¹³C NMR (151 MHz, DMSO) δ 165.89, 139.90, 139.02, 135.46, 132.36, 128.89, 128.47, 128.14, 126.85, 124.58, 121.38, 119.63, 117.98, 113.22, 103.77, 52.04.

### Synthesis of Intermediate 63-v

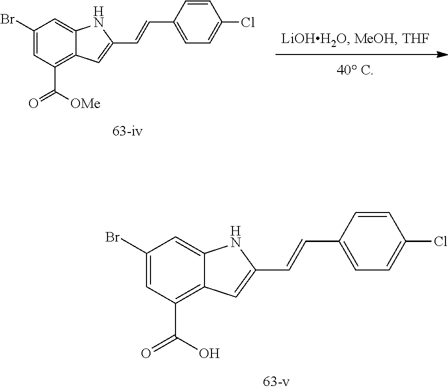 [SMILES: COC(=O)c1cc(Br)cc2[nH]c(/C=C/c3ccc(Cl)cc3)cc12 ; O=C(O)c1cc(Br)cc2[nH]c(/C=C/c3ccc(Cl)cc3)cc12]

### Intermediate 63-v: (E)-6-Bromo-2-(4-chlorostyryl)-1H-indole-4-carboxylic Acid

To a stirred solution of intermediate 63-iv (39 mg, 0.1 mmol) in MeOH (1 mL) and THF (1 mL) was added a solution of LiOH·H₂O (22 mg, 0.5 mmol) in H₂O (1 mL) and the mixture was heated at 40° C. for 21 h. The mixture was acidified with 1M HCl to pH 1 and then extracted with EtOAc (×2). The combined organic phase was washed with brine, dried over anhydrous Na₂SO₄, filtered and concentrated. The crude product was purified by silica gel chromatography eluting with a MeOH/DCM gradient to provide intermediate 63-v (34 mg, 90%). ¹H NMR (400 MHz, DMSO) δ 13.03 (s, 1H), 11.89 (s, 1H), 7.77 (s, 2H), 7.66 (d, J=8.6 Hz, 2H), 7.51 (d, J=8.5 Hz, 2H), 7.36 (dd, J=36.8, 16.5 Hz, 2H), 7.18 (d, J=1.7 Hz, 1H). Mass calculated for (C₁₇H₁₁BrClNO₂−H)⁻ 375.96, found 376.0.

### Compound 63: (E)-N-Benzyl-6-bromo-2-(4-chlorostyryl)-1H-indole-4-carboxamide

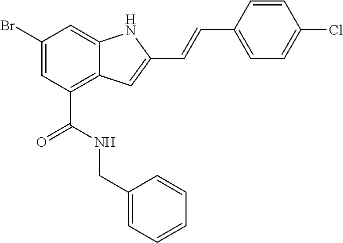 [SMILES: O=C(NCc1ccccc1)c1cc(Br)cc2[nH]c(/C=C/c3ccc(Cl)cc3)cc12]

Prepared according to general method XIII from 63-v and benzylamine. ¹H NMR (400 MHz, DMSO) δ 11.75 (s, 1H), 9.01 (t, J=6.0 Hz, 1H), 7.63 (dd, J=10.4, 4.1 Hz, 4H), 7.47 (d, J=8.5 Hz, 2H), 7.40-7.21 (m, 7H), 7.08 (s, 1H), 4.51 (d, J=6.0 Hz, 2H). Mass calculated for (C₂₄H₁₈BrClN₂O−H)⁻ 463.0, found 463.0.

### Compound 64: (E)-6-Bromo-2-(4-chlorostyryl)-N,N-dimethyl-1H-indole-4-carboxamide

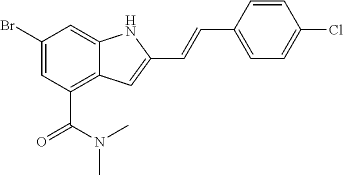 [SMILES: CN(C)C(=O)c1cc(Br)cc2[nH]c(/C=C/c3ccc(Cl)cc3)cc12]

Prepared according to general method XIII from 63-v and dimethylamine (10 mg, 37%). ¹H NMR (400 MHz, DMSO) δ 11.76 (s, 1H), 7.66-7.55 (m, 3H), 7.47 (d, J=8.5 Hz, 2H), 7.27 (d, J=4.7 Hz, 2H), 7.10 (d, J=1.6 Hz, 1H), 6.54 (s, 1H), 3.06 (s, 3H), 2.87 (s, 3H). Mass calculated for (C₁₉H₁₆BrClN₂O−H)⁻ 403.0, found 403.0.

### Synthesis of Compound 65

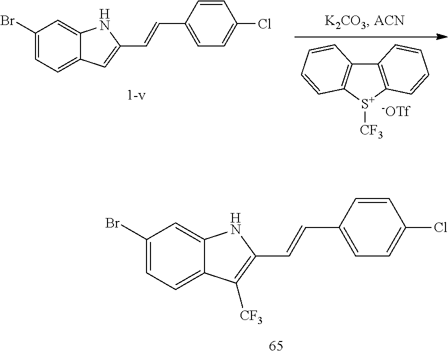 [SMILES: Clc1ccc(/C=C/c2cc3ccc(Br)cc3[nH]2)cc1 ; FC(F)(F)[s+]1c2ccccc2c2ccccc21 ; FC(F)(F)c1c(/C=C/c2ccc(Cl)cc2)[nH]c2cc(Br)ccc12 ; "-OTf"]

### Compound 65: (E)-6-Bromo-2-(4-chlorostyryl)-3-(trifluoromethyl)-1H-indole (67)

A mixture of intermediate 1-v (60 mg, 0.18 mmol), 5-(trifluoromethyl) dibenzothiophenium trifluoromethanesulfonate (78 mg, 0.19 mmol) and K₂CO₃ (40 mg, 0.29 mmol) in ACN (4 mL) was heated at 50° C. under Ar for 24 hrs and then concentrated under reduced pressure. The crude product was purified by silica gel chromatography, eluting with an EtOAc/hexanes gradient, to provide the compound 65 (25 mg, 34%). ¹H NMR (600 MHz, CDCl₃) δ 8.53 (s, 1H), 7.62 (d, J=8.6 Hz, 1H), 7.56 (s, 1H), 7.49 (d, J=8.3 Hz, 2H), 7.41 (d, J=8.2 Hz, 2H), 7.35 (d, J=1.6 Hz, 1H), 7.33 (d, J=7.2 Hz, 1H), 7.29 (d, J=0.9 Hz, 1H), 7.00 (d, J=16.6 Hz, 1H). Mass calculated for (C₂₃H₁₅BrClNO−H)⁻ 436.0, found 435.9.

### Compound 66: 6-Bromo-2-(4-chlorophenethyl)-3-(trifluoromethyl)-1H-indole

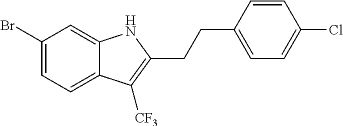 [SMILES: FC(F)(F)c1c(CCc2ccc(Cl)cc2)[nH]c2cc(Br)ccc12]

Prepared according to general method IV from compound 65 (18 mg, 64%). ¹H NMR (400 MHz, CDCl₃) δ 7.89 (bs, 1H), 7.58 (d, J=8.5 Hz, 1H), 7.44 (d, J=1.5 Hz, 1H), 7.32 (dd, J=8.6, 1.7 Hz, 1H), 7.30-7.26 (m, 2H), 7.08 (d, J=8.4 Hz, 2H), 3.19 (t, J=7.5 Hz, 2H), 3.01 (t, J=7.5 Hz, 2H). Mass calculated for (C₁₇H₁₂BrClF₃N−H)⁻ 402.0, found 401.9.

### Synthesis of Compound 67

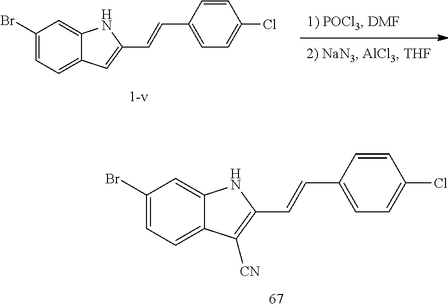 [SMILES: Clc1ccc(/C=C/c2cc3ccc(Br)cc3[nH]2)cc1 ; N#Cc1c(/C=C/c2ccc(Cl)cc2)[nH]c2cc(Br)ccc12]

### Compound 67: (E)-6-Bromo-2-(4-chlorostyryl)-1H-indole-3-carbonitrile

To a cooled DMF (3 mL) at 0° C. under Ar was added POCl₃ (35 uL, 0.38 mmol) and the mixture was allowed to warm to rt followed by the addition of compound 1-v (92 mg, 0.28 mmol) in DMF (1 mL). The mixture was heated at 35° C. for 2 h, diluted with EtOAc and washed with H₂O and brine. The organic phase was dried over anhydrous Na₂SO₄ and concentrated under reduced pressure. The residue was partially purified by silica gel chromatography, eluting with an EtOAc/hexanes gradient, and then it was added along with NaN₃ (58 mg, 0.89 mmol) to a stirred suspension of AlC₃ (40 mg, 0.3 mmol) in THF (4 mL). The mixture was refluxed under Ar for 1 d, quenched with H₂O and extracted with EtOAc (×2). The combined organic layer was concentrated under reduced pressure and purified by silica gel chromatography, eluting with an EtOAc/hexanes gradient to provide compound 67 (27 mg, 27%). ¹H NMR (600 MHz, DMSO) δ 7.73 (d, J=8.5 Hz, 2H), 7.69 (dd, J=1.2, 0.5 Hz, 1H), 7.57 (d, J=8.4 Hz, 1H), 7.56 (d, J=16.5 Hz, 1H), 7.52 (d, J=8.5 Hz, 2H), 7.38-7.36 (m, 1H), 7.29 (d, J=16.5 Hz, 1H). Mass calculated for (C₁₇H₁₀BrClN₂—H) 357.0, found 357.0.

### Synthesis of Compound 68

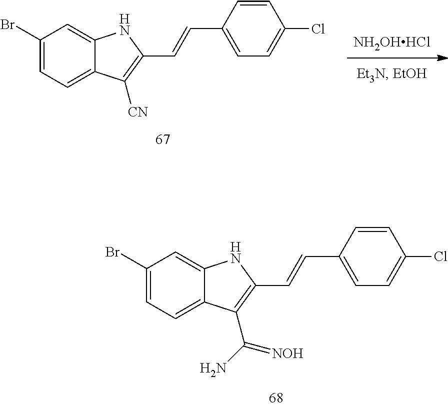

### Compound 68: 6-Bromo-2-((E)-4-chlorostyryl)-N′-hydroxy-1H-indole-3-carboximidamide

A mixture of compound 67 (50 mg, 0.14 mmol), hydroxylamine hydrochloride (100 mg, 1.4 mmol) and Et₃N (200 uL, 1.4 mmol) in EtOH (2 mL) was heat in a sealed tube at 80° C. for 16 h and then diluted with EtOAc. The mixture was washed with H₂O (×2), brine, dried over anhydrous Na₂SO₄, filtered and concentrated under reduced pressure. The crude product was purified by silica gel chromatography, eluting with an EtOAc/hexanes gradient, to provide compound 68 (19 mg, 35%). ¹H NMR (400 MHz, DMSO) δ 11.75 (s, 1H), 9.59 (s, 1H), 7.68 (d, J=8.5 Hz, 1H), 7.61-7.44 (m, 6H), 7.24 (d, J=16.7 Hz, 1H), 7.18 (dd, J=8.5, 1.8 Hz, 1H), 5.77 (s, 2H). Mass calculated for (C₁₇H₁₃BrClN₃O−H)⁻ 390.0, found 390.0.

### Synthesis of Compound 69

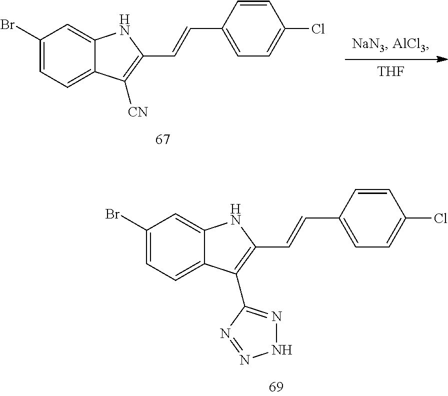 [SMILES: N#Cc1c(/C=C/c2ccc(Cl)cc2)[nH]c2cc(Br)ccc12 ; "NaN3, AlCl3," ; Clc1ccc(/C=C/c2[nH]c3cc(Br)ccc3c2-c2nn[nH]n2)cc1 ; "THF"]

### Compound 69: (E)-6-Bromo-2-(4-chlorostyryl)-3-(2H-tetrazol-5-yl)-1H-indole

A mixture of compound 67 (50 mg, 0.14 mmol) and NaN₃ (39 mg, 0.6 mmol) was added to a stirred suspension of AlCl₃ (30 mg, 0.23 mmol) in THF (0.5 mL). The resulting mixture was heated in a sealed tube at 90° C. for 3 d and then diluted with EtOAc. The organic layer was washed with H₂O (×2), brine, dried over anhydrous Na₂SO₄, filtered and concentrated under reduced pressure. The crude product was purified by silica gel chromatography, eluting with a MeOH/DCM gradient, to provide compound 69 (24 mg, 43%). ¹H NMR (600 MHz, DMSO) δ 12.25 (s, 1H), 8.00 (d, J=8.5 Hz, 1H), 7.89 (d, J=16.6 Hz, 1H), 7.67 (d, J=8.4 Hz, 2H), 7.64 (d, J=0.6 Hz, 1H), 7.52 (d, J=8.3 Hz, 2H), 7.46 (d, J=16.5 Hz, 1H), 7.33 (dd, J=8.5, 0.9 Hz, 1H). Mass calculated for (C₁₇H₁₁BrClN₅−H)⁻ 400.0, found 399.9.

### Synthesis of Compound 70

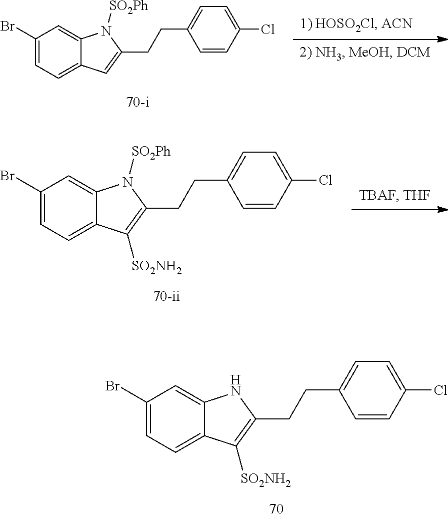

### Compound 70: 6-Bromo-2-(4-chlorophenethyl)-1H-indole-3-sulfonamide

To a stirred solution of 70-i (prepared from 1-iv using general method IV) (50 mg, 0.11 mmol) in ACN (3 mL) under Ar was added HOSO₂Cl (0.1 mL, 1.5 mmol) dropwise and the resulting mixture was stirred at rt for 3 d. The mixture was poured into ice water and extracted with DCM (×3). The organic phase was washed with saturated aqueous NaHCO₃, brine, dried over anhydrous Na₂SO₄, filtered and concentrated under reduced pressure. The residue was suspended in DCM (5 mL) followed by the addition of NH₃ solution (2M in MeOH, 0.6 mL, 1.2 mmol). The mixture was stirred at rt for 16 h and the concentrated under reduced pressure. The residue was purified by silica chromatography, eluting with a MeOH/DCM gradient, to provide the corresponding sulfonamide intermediate 70-ii which was further dissolved in THF (5 mmol) followed by the addition of TBAF (1M in THF, 0.14 mL, 0.14 mmol). The mixture was stirred at rt for 20 h and then concentrated under reduced pressure. The crude product was purified by preparative HPLC (ACN/H₂O with 0.1% formic acid) to provide compound 70 (14 mg, 37%). ¹H NMR (600 MHz, DMSO) δ 11.87 (s, 1H), 7.79 (d, J=8.6 Hz, 1H), 7.58 (d, J=1.7 Hz, 1H), 7.38 (d, J=8.3 Hz, 2H), 7.30 (d, J=8.5 Hz, 2H), 7.28 (dd, J=8.7, 1.8 Hz, 1H), 7.17 (s, 2H), 3.29-3.25 (m, 2H), 3.02-2.97 (m, 2H). Mass calculated for (C₁₋₆H₁₄BrClN₂O₂S−H)⁻ 413.0, found 412.9.

### Synthesis of Compound 71

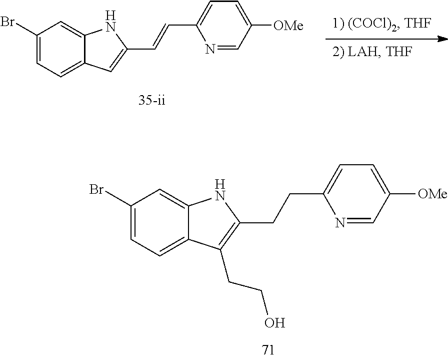

### Compound 71: 2-(6-Bromo-2-(2-(5-methoxypyridin-2-yl)ethyl)-1H-indol-3-yl)ethanol

To a stirred solution of intermediate 35-ii (50 mg, 0.15 mmol) in THF (6 mL) under Ar was added oxalylchloride (0.2 mL, 2.3 mmol) and the mixture was heated at 50° C. for 4 h. The reaction was quenched with MeOH (5 mL) and then diluted with EtOAc. The mixture was washed with H₂O, brine, dried over anhydrous Na₂SO₄ and concentrated under reduced pressure. The residue was dissolved in THF (6 mL) followed by the addition of LAH (50 mg, 1.3 mmol) slowly. The resulting mixture was refluxed for 3.5 h, cooled to rt and slowly quenched with H₂O. The mixture was diluted with EtOAc and the resulting organic layer was washed with H₂O, brine, dried over anhydrous Na₂SO₄, filtered and concentrated under reduced pressure. The crude product was purified by preparative HPLC (ACN/H₂O with 0.1% formic acid) to provide compound 71 (9 mg, 16%). ¹H NMR (400 MHz, MeOD) δ 8.16 (d, J=2.9 Hz, 1H), 7.41 (d, J=1.6 Hz, 1H), 7.33 (d, J=8.4 Hz, 1H), 7.28 (dd, J=8.6, 3.0 Hz, 1H), 7.11-7.03 (m, 2H), 3.85 (s, 3H), 3.56 (t, J=7.4 Hz, 2H), 3.10 (s, 4H), 2.78 (t, J=7.4 Hz, 2H). Mass calculated for (C₁₈H₁₉BrN₂O₂+H)⁺375.1, found 375.0.

### Synthesis of Compound 72 and 73

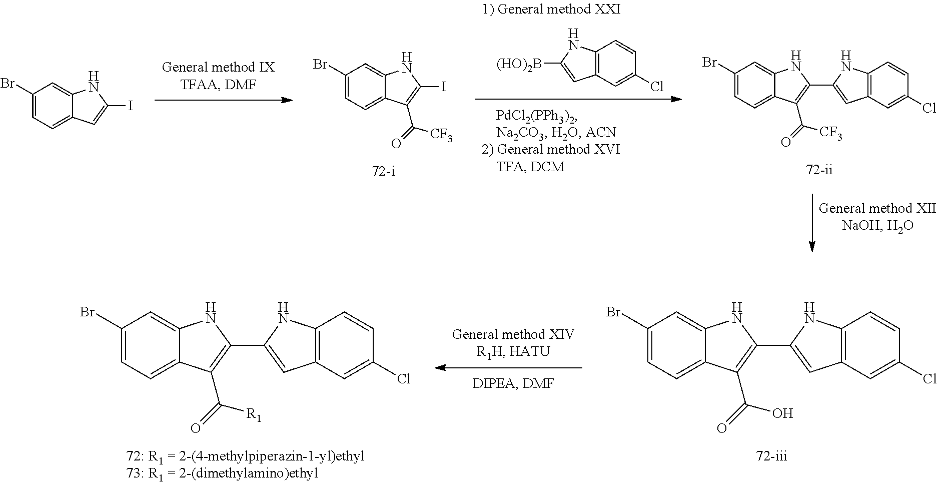

### Synthesis of Intermediate 72-i

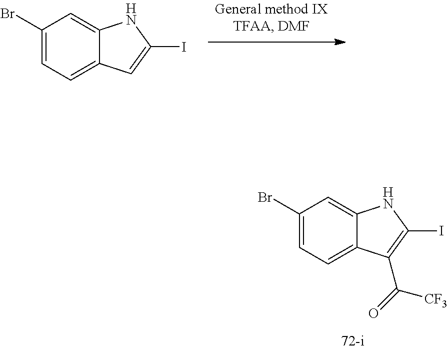 [SMILES: Brc1ccc2cc(I)[nH]c2c1 ; O=C(c1c(I)[nH]c2cc(Br)ccc12)C(F)(F)F]

### Intermediate 72-i: 1-(6-bromo-2-iodo-1H-indol-3-yl)-2,2,2-trifluoroethan-1-one

Prepared according to the general method IX from 6-bromo-2-iodoindole (880 mg, 97%). ¹H NMR (400 MHz, CDCl₃) δ 8.99 (s, 1H), 7.89 (d, J=8.8 Hz, 1H), 7.60 (d, J=1.5 Hz, 1H), 7.43 (dd, J=8.8, 1.8 Hz, 1H).

### Synthesis of Intermediate 72-ii

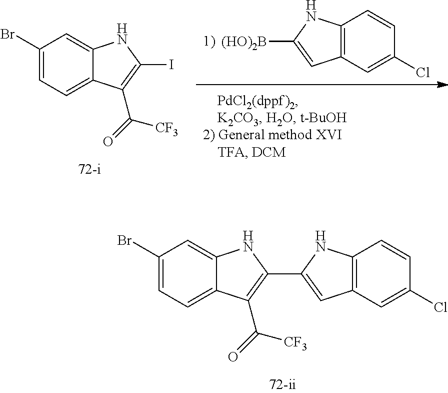 [SMILES: O=C(c1c(I)[nH]c2cc(Br)ccc12)C(F)(F)F ; OB(O)c1cc2cc(Cl)ccc2[nH]1 ; O=C(c1c(-c2cc3cc(Cl)ccc3[nH]2)[nH]c2cc(Br)ccc12)C(F)(F)F]

### Intermediate 72-ii: 1-(6-Bromo-5′-chloro-1H,1′H-[2,2′-biindol]-3-yl)-2,2,2-trifluoroethanone

A solution of intermediate 72-i (1.00 g, 2.4 mmol) and (6-chloro-1H-indol-2-yl)boronic acid (1.06 g, 3.6 mmol) in t-BuOH (200 mL) was purged with nitrogen for 15 min followed by the addition of K₂CO₃ (1.5 M aqueous solution, 8.37 mmol). The mixture was further purged with nitrogen for 5 min and then PdCl₂(dppf)₂ (525 mg, 0.71 mmol) was added. The reaction mixture was heated at 50° C. for 5 h and then diluted with EtOAc. The organic layer was washed with H₂O (×2), brine, dried over anhydrous Na₂SO₄, filtered and concentrated under reduced pressure. The residue was purified by silica gel chromatography, eluting with EtOAc/hexanes gradient, to provide boc protected intermediate. The boc group was removed using general method XVI to give intermediate 72-ii (600 mg, 57%). ¹H NMR (400 MHz, DMSO) δ 13.17 (s, 1H), 11.94 (s, 1H), 7.86 (d, J=8.6 Hz, 1H), 7.78 (d, J=2.1 Hz, 1H), 7.74 (d, J=1.7 Hz, 1H), 7.58 (d, J=8.7 Hz, 1H), 7.51 (dd, J=8.7, 1.9 Hz, 1H), 7.25 (dd, J=8.7, 2.1 Hz, 1H), 7.15 (d, J=1.3 Hz, 1H). ¹³C NMR (151 MHz, DMSO) δ 175.17 (q, J_{C,F}=35.6 Hz), 141.58, 137.52, 135.90, 129.23, 128.73, 126.60, 125.04, 124.93, 123.95, 122.42 (q, J_{C,F}=3.9 Hz), 120.46, 117.17, 117.17 (q, J_{C,F}=290.5 Hz), 115.60, 114.14, 107.32, 105.88. Mass calculated for (C₁₈H₉BrClF₃N₂O−H)⁻ 441.0, found 441.0.

### Synthesis of Intermediate 72-iii

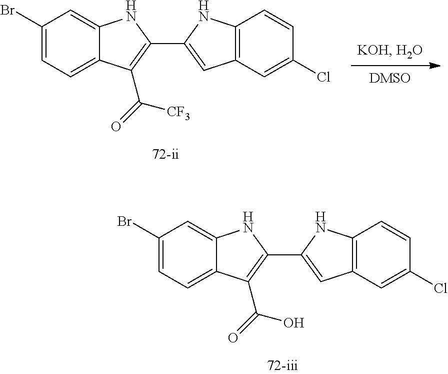

### Intermediate 72-iii: 6-Bromo-5′-chloro-1H,1′H-[2,2′-biindole]-3-carboxylic Acid

KOH (20% aqueous solution, 150 mL) was added to a stirred solution of intermediate 72-ii (2.08 g, 4.72 mmol) in DMSO (150 mL). The mixture was heated at 60° C. for 1 h and then diluted with H₂O. The resulting solution was acidified with 1M HCl to pH 2-3 and the precipitation was collected by filtration. The solid was washed with H₂O and further purified by silica gel chromatography, eluting with MeOH/DCM gradient, to provide intermediate 72-iii (1.60 g, 87%). ¹H NMR (600 MHz, DMSO) δ 12.34 (s, 1H), 8.13 (d, J=8.5 Hz, 1H), 7.73 (d, J=1.8 Hz, 1H), 7.63-7.56 (m, 2H), 7.30 (d, J=8.7 Hz, 1H), 7.23 (s, 1H), 7.17 (dd, J=8.6, 1.9 Hz, 1H). Mass calculated for (C₁₇H₁₀BrClN₂O₂−H)⁻ 389.0, found 388.9.

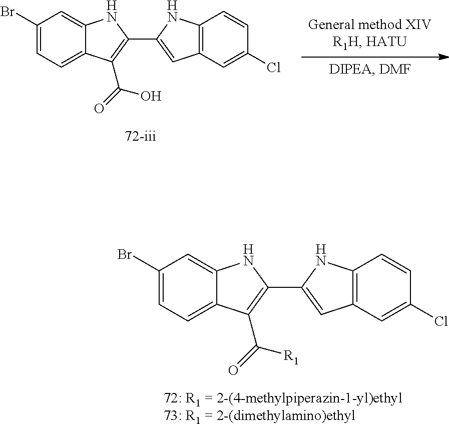

### Compound 72: 6-Bromo-5′-chloro-N-(2-(4-methylpiperazin-1-yl)ethyl)-1H,1′H-[2,2′-biindole]-3-carboxamide

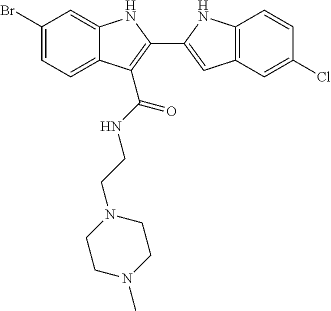 [SMILES: CN1CCN(CCNC(=O)c2c(-c3cc4cc(Cl)ccc4[nH]3)[nH]c3cc(Br)ccc23)CC1]

Prepared according to general method XIV from 72-iii and 2-(4-methylpiperazin-1-yl)ethan-1-amine (43 mg, 65%). ¹H NMR (400 MHz, DMSO-d₆) δ 12.54 (s, 1H), 12.30 (s, 1H), 8.09 (t, J=5.5 Hz, 1H), 7.93 (d, J=8.6 Hz, 1H), 7.73 (d, J=2.0 Hz, 1H), 7.63 (d, J=1.8 Hz, 1H), 7.60 (d, J=8.7 Hz, 1H), 7.31 (dd, J=8.6, 1.8 Hz, 1H), 7.19-7.13 (m, 2H), 3.51 (q, J=6.2 Hz, 2H), 2.57 (t, J=6.4 Hz, 2H), 2.38 (s, 6H), 2.19 (s, 3H). Mass calculated for (C₂₄H₂₅BrClN₅O+H)⁺516.1, found 516.0.

### Compound 73: 6-Bromo-5′-chloro-N-(2-(dimethylamino)ethyl)-1H,1′H-[2,2′-biindole]-3-carboxamide

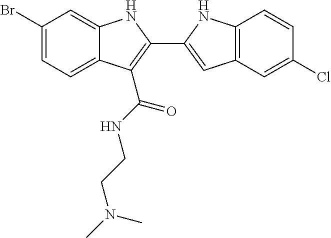 [SMILES: CN(C)CCNC(=O)c1c(-c2cc3cc(Cl)ccc3[nH]2)[nH]c2cc(Br)ccc12]

Prepared according to general method XIV from 72-iii and N¹,N¹-dimethylethane-1,2-diamine (45 mg, 76%). ¹H NMR (400 MHz, DMSO-d₆) δ 12.51 (s, 1H), 12.29 (s, 1H), 8.12 (t, J=5.6 Hz, 1H), 7.80 (d, J=8.6 Hz, 1H), 7.73 (d, J=2.0 Hz, 1H), 7.64-7.59 (m, 2H), 7.33 (dd, J=8.6, 1.8 Hz, 1H), 7.19-7.11 (m, 2H), 3.50 (q, J=6.4 Hz, 2H), 2.56-2.52 (m, 2H), 2.24 (s, 6H). Mass calculated for (C₂₁H₂₀BrClN₄O+H)⁺461.1, found 461.0.

### Synthesis of Compound 74

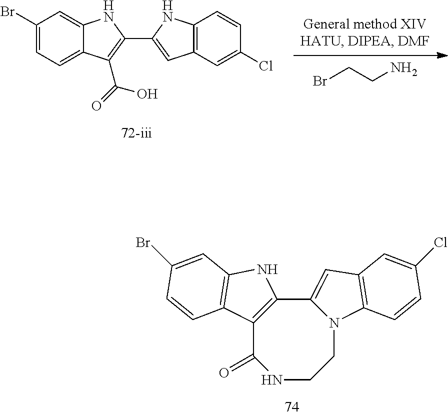

### Compound 74: 12-Bromo-2-chloro-7,8-dihydro-6H-[1,4]diazocino[1,8-a:7,6-b′]diindol-9(14H)-one

Prepared according to general method XIV from 72-iii and 2-bromoethan-1-amine (32 mg, 60%). ¹H NMR (400 MHz, DMSO-d₆) δ 14.73 (s, 1H), 12.48 (s, 1H), 7.99 (d, J=8.7 Hz, 1H), 7.75 (d, J=2.0 Hz, 1H), 7.64-7.58 (m, 2H), 7.31 (dd, J=8.7, 1.9 Hz, 1H), 7.27 (t, J=1.2 Hz, 1H), 7.18 (dd, J=8.7, 2.1 Hz, 1H), 4.59 (t, J=9.5 Hz, 2H), 4.19 (t, J=9.5 Hz, 2H). Mass calculated for (C₁₉H₁₃BrClN₃O+H)⁺414.0, found 413.9.

### Synthesis of Intermediates 75-i and 75-ii

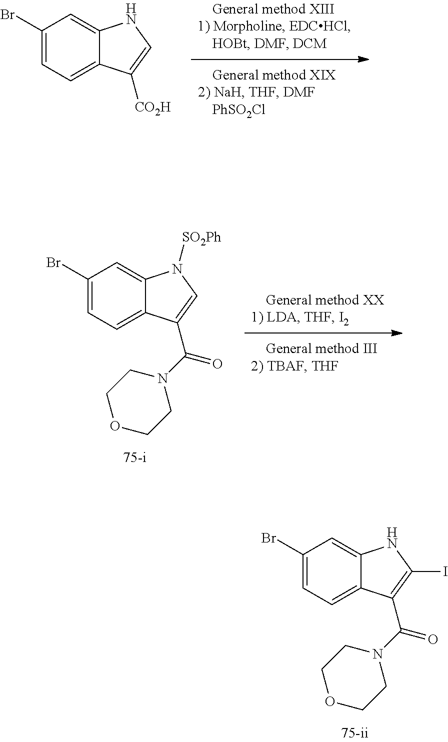

### Intermediate 75-i: (6-Bromo-1-(phenylsulfonyl)-1H-indol-3-yl)(morpholino)methanone

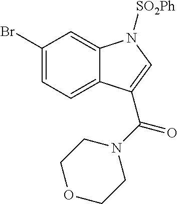

Prepared according to general method XIII and XIX from 6-bromo-1H-indole-3-carboxylic acid and morpholine (1.32 g, 59%). ¹H NMR (400 MHz, DMSO) δ 8.19 (s, 1H), 8.14-8.07 (m, 3H), 7.80-7.74 (m, 1H), 7.70-7.64 (m, 2H), 7.62 (d, J=8.5 Hz, 1H), 7.51 (dd, J=8.5, 1.7 Hz, 1H), 3.62 (bs, 4H), 3.57 (bs, 4H).

### Intermediate 75-ii: (6-Bromo-2-iodo-1H-indol-3-yl)(morpholino)methanone

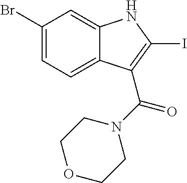 [SMILES: O=C(c1c(I)[nH]c2cc(Br)ccc12)N1CCOCC1]

Prepared according to general method XX and III from 75-i (120 mg, 83%).

### Synthesis of Compounds 75 and 76

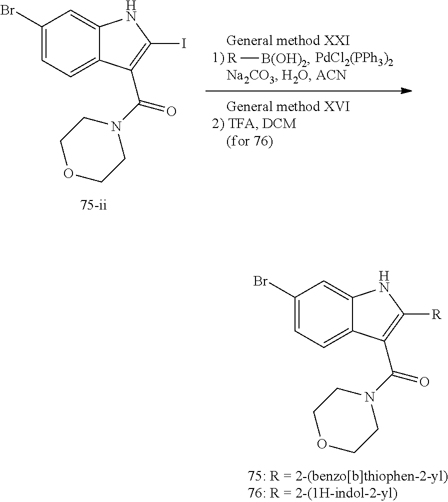

### Compound 75: 2-(Benzo[b]thiophen-2-yl)-6-bromo-1H-indol-3-yl)(morpholino)methanone

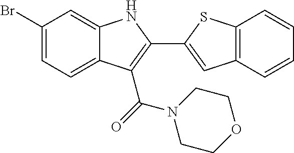 [SMILES: O=C(c1c(-c2cc3ccccc3s2)[nH]c2cc(Br)ccc12)N1CCOCC1]

Prepared according to general method XXI from 75-ii and benzo[b]thiophen-2-ylboronic acid (16 mg, 42%). ¹H NMR (400 MHz, DMSO) δ 12.20 (s, 1H), 8.06-8.03 (m, 1H), 7.95 (dd, J=6.6, 2.2 Hz, 1H), 7.85 (s, 1H), 7.62 (d, J=1.5 Hz, 1H), 7.47-7.40 (m, 3H), 7.26 (dd, J=8.5, 1.8 Hz, 1H), 4.00-3.21 (m, 4H). Mass calculated for (C₂₁H₁₇BrN₂O₂S−H)⁻ 439.0, found 438.9.

### Compound 76: (6-Bromo-1H,1′H-[2,2′-biindol]-3-yl)(morpholino)methanone

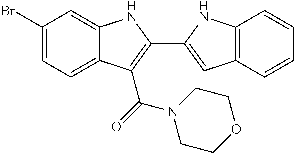 [SMILES: O=C(c1c(-c2cc3ccccc3[nH]2)[nH]c2cc(Br)ccc12)N1CCOCC1]

Prepared according to general method XXI and XVI from 75-ii and (1-(tert-butoxycarbonyl)-1H-indol-2-yl)boronic acid (15 mg, 44%). ¹H NMR (500 MHz, CDCl₃) δ 10.87 (s, 1H), 8.67 (d, J=32.3 Hz, 1H), 7.63 (d, J=7.9 Hz, 1H), 7.56 (d, J=1.4 Hz, 1H), 7.47 (d, J=8.5 Hz, 1H), 7.36 (d, J=8.5 Hz, 1H), 7.31 (dd, J=8.5, 1.6 Hz, 1H), 7.27-7.23 (m, 3H), 7.16-7.11 (m, 1H), 6.84 (d, J=1.2 Hz, 1H), 4.42-2.77 (m, 8H). Mass calculated for (C₂₁H₁₈BrN₃O₂−H)⁻ 422.0, found 422.0.

### Synthesis of Intermediates 77-i and 77-ii

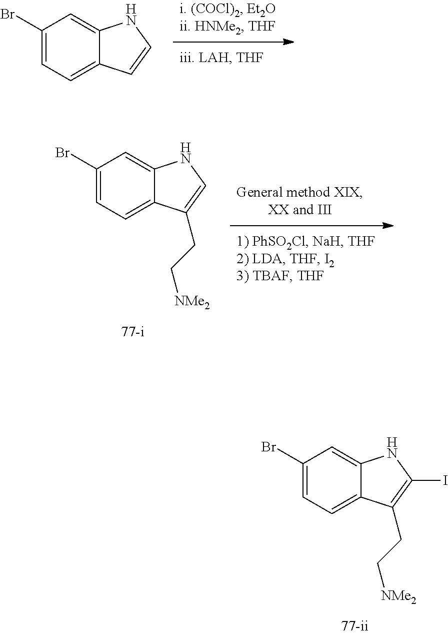 [SMILES: Brc1ccc2cc[nH]c2c1 ; CN(C)CCc1c[nH]c2cc(Br)ccc12 ; CN(C)CCc1c(I)[nH]c2cc(Br)ccc12]

### Intermediate 77-i: 2-(6-bromo-1H-indol-3-yl)-N,N-dimethylethan-1-amine

To a stirred solution of 6-bromoindole (5.0 g, 25.5 mmol) in Et₂O (50 mL) at 0° C. under Ar was added oxalyl chloride (2.7 mL, 30.9 mmol) gradually. The mixture was stirred at 0° C. for 3 h followed by the addition of dimethylamine (2M in THF, 40 mL, 80 mmol). The resulting mixture was stirred at rt for 2 h and then concentrated under reduced pressure. The residue was triturated with H₂O and the solid was collected by filtration. The solid was suspended in THF (100 mL) under Ar followed by the addition of LAH (2.0 g, 52.6 mmol) gradually and the mixture was refluxed for 20 h. After cooling to rt, NaOH (15% aqueous solution, 30 mL) was added dropwise. The mixture was filtered through a pad of celite and then concentrated under reduced pressure. The crude product was purified by silica gel chromatography, eluting with an EtOAc/hexanes gradient, to provide intermediate 77-i (3.17 g, 47%). ¹H NMR (400 MHz, Chloroform-d) δ 8.19 (s, 1H), 7.54 (d, J=1.7 Hz, 1H), 7.49 (d, J=8.4 Hz, 1H), 7.24 (dd, J=8.4, 1.7 Hz, 1H), 7.05 (d, J=2.2 Hz, 1H), 3.05 (t, J=8.0 Hz, 2H), 2.87-2.72 (m, 2H), 2.49 (s, 6H).

### Intermediate 77-ii. 2-(6-bromo-2-iodo-1H-indol-3-yl)-N,N-dimethylethan-1-amine

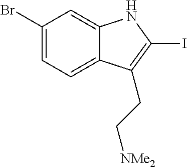 [SMILES: CN(C)CCc1c(I)[nH]c2cc(Br)ccc12]

Prepared according to general method XIX, XX and III respectively from 77-i (32 mg, 3%). Mass calculated for (C₁₂H₁₄BrIN₂+H)⁺392.9, found 393.0.

### Synthesis of Compound 77

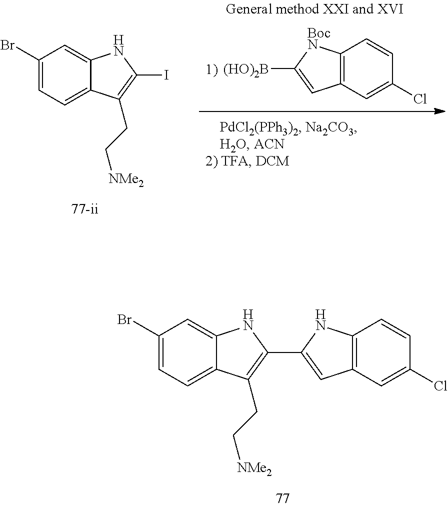 [SMILES: CN(C)CCc1c(I)[nH]c2cc(Br)ccc12 ; CC(C)(C)OC(=O)n1c(B(O)O)cc2cc(Cl)ccc21 ; CN(C)CCc1c(-c2cc3cc(Cl)ccc3[nH]2)[nH]c2cc(Br)ccc12]

### Compound 77: 2-(6-Bromo-5′-chloro-1H,1′H-[2,2′-biindol]-3-yl)-N,N-dimethylethan-1-amine

Prepared according to general method XXI and XVI from 77-ii and (1-(tert-butoxycarbonyl)-5-chloro-1H-indol-2-yl)boronic acid (5 mg, 16%). ¹H NMR (400 MHz, Methanol-d₄) δ 7.56 (dd, J=7.0, 1.9 Hz, 2H), 7.50 (d, J=8.5 Hz, 1H), 7.40 (d, J=8.6 Hz, 1H), 7.19 (dd, J=8.4, 1.7 Hz, 1H), 7.12 (dd, J=8.6, 2.1 Hz, 1H), 6.74 (d, J=1.0 Hz, 1H), 3.19 (t, J=7.5 Hz, 2H), 2.72 (t, J=7.5 Hz, 2H), 2.42 (s, 6H). Mass calculated for (C₂₀H₁₉BrClN₃+H)⁺418.0, found 418.0.

### Synthesis of Intermediates 78-i, 78-ii and 78-iii

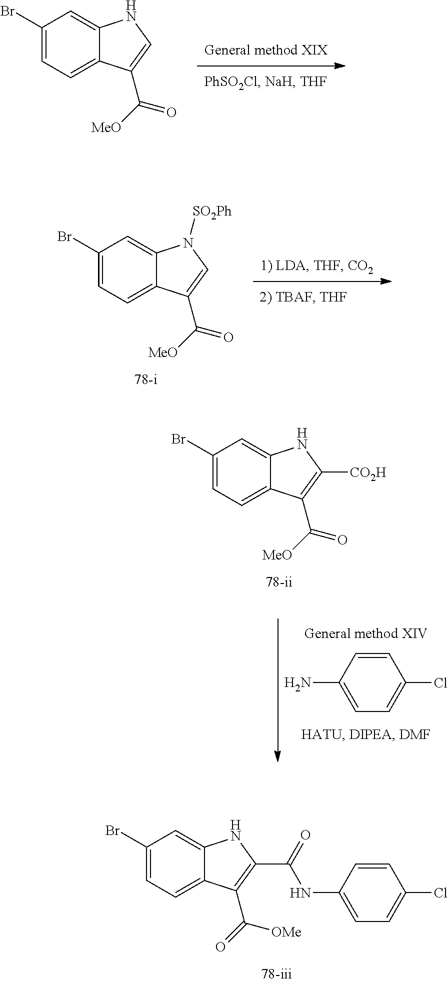

### Intermediate 78-i: Methyl 6-bromo-1-(phenylsulfonyl)-1H-indole-3-carboxylate

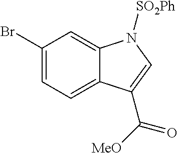

Prepared according to general method XIX from methyl 6-bromo-1H-indole-3-carboxylate (6.61 g, 92%). ¹H NMR (400 MHz, DMSO-d₆) δ 8.53 (s, 1H), 8.26-8.20 (m, 2H), 8.13 (d, J=1.7 Hz, 1H), 8.00 (d, J=8.6 Hz, 1H), 7.84-7.76 (m, 1H), 7.69 (dd, J=8.5, 7.3 Hz, 2H), 7.60 (dd, J=8.5, 1.7 Hz, 1H), 3.88 (s, 3H).

### Intermediate 78-ii: 6-Bromo-3-(methoxycarbonyl)-1H-indole-2-carboxylic Acid

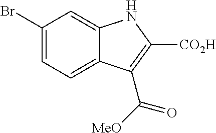 [SMILES: COC(=O)c1c(C(=O)O)[nH]c2cc(Br)ccc12]

To a stirred solution of 78-i (6.61 g, 16.8 mmol) in anhydrous THF (125 mL) at −78° C. was added a solution of LDA (2.0 M in THF/heptane/ethylbenzene, 12.0 ml, 24 mmol) dropwise. The mixture was stirred at 0° C. for 15 min and then CO₂ gas was bubbled through for 30 min. The reaction was quenched with H₂O and then diluted with EtOAc. The resulting mixture was washed with 0.5M aqueous HCl, brine, dried over anhydrous Na₂SO₄, filtered and concentrated in vacuo. The residue was dissolved in THF (150 ml) followed by the addition of TBAF (50.0 mL, 1 M in THF, 50 mmol). The mixture was stirred at rt for 18 h and then diluted with EtOAc. The mixture was washed with H₂O, brine, dried over anhydrous Na₂SO₄, filtered and concentrated under reduced pressure. The crude product was partially purified by silica gel chromatography, eluting with a MeOH/DCM gradient, and then recrystallized with EtOAc/hexanes to provide intermediate 78-ii as a brown solid (2.85 g, 57%). ¹H NMR (400 MHz, DMSO-d₆) δ 14.42 (s, 1H), 12.91 (s, 1H), 7.95 (d, J=8.7 Hz, 1H), 7.72 (d, J=1.8 Hz, 1H), 7.44 (dd, J=8.7, 1.8 Hz, 1H), 3.97 (s, 3H).

### Intermediate 78-iii: Methyl 6-bromo-2-((4-chlorophenyl)carbamoyl)-1H-indole-3-carboxylate

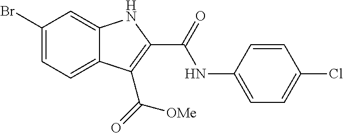 [SMILES: COC(=O)c1c(C(=O)Nc2ccc(Cl)cc2)[nH]c2cc(Br)ccc12]

Prepared according to general method XIV from 78-ii and the corresponding amine (1.15 g, 84%). ¹H NMR (400 MHz, DMSO-d₆) δ 12.95 (s, 1H), 12.39 (s, 1H), 8.06 (d, J=8.8 Hz, 1H), 7.80 (d, J=8.9 Hz, 2H), 7.77 (d, J=1.8 Hz, 1H), 7.51 (d, J=8.8 Hz, 2H), 7.44 (dd, J=8.8, 1.9 Hz, 1H), 4.00 (s, 3H).

### General Method XXII

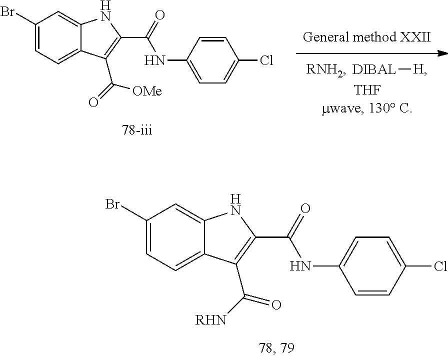

To a stirred solution of the corresponding amine (3.5 mmol) in THF (8 mL) under Ar was added DIBAL-H (1M in cyclohexane, 3.5 mmol) gradually and the mixture was stirred at rt for 15 min. A suspension of 78-iii (1.0 mmol) in THF (8 mL) was added and the resulting mixture was heated with microwave at 130° C. for 30 min. The mixture was diluted with EtOAc (200 mL) and saturated aqueous solution of sodium citrate (200 mL). The mixture was vigorously stirred for 1 h and the organic phase was washed with brine, dried over anhydrous Na₂SO₄, filtered and concentrated under reduced pressure. The crude product was purified by silica gel chromatography, eluting with a MeOH/DCM gradient, to give the desired adduct.

### Compound 78: tert-Butyl 4-(2-(6-bromo-2-((4-chlorophenyl)carbamoyl)-1H-indole-3-carboxamido)ethyl)piperazine-1-carboxylate

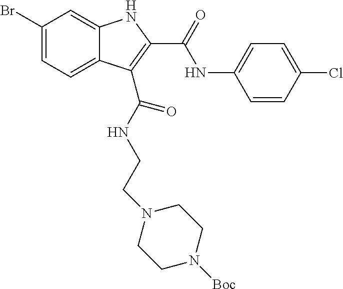 [SMILES: CC(C)(C)OC(=O)N1CCN(CCNC(=O)c2c(C(=O)Nc3ccc(Cl)cc3)[nH]c3cc(Br)ccc23)CC1]

Prepared according to general method XXII from 78-iii and tert-butyl 4-(2-aminoethyl)piperazine-1-carboxylate (99 mg, 67%). ¹H NMR (400 MHz, DMSO) δ 13.15 (s, 1H), 12.59 (s, 1H), 8.62 (t, J=5.3 Hz, 1H), 8.00 (d, J=8.8 Hz, 1H), 7.80-7.70 (m, 3H), 7.49 (d, J=8.8 Hz, 2H), 7.43 (dd, J=8.8, 1.7 Hz, 1H), 3.61-3.49 (m, 2H), 3.33 (bs, 4H), 2.60 (t, J=6.2 Hz, 2H), 2.47-2.39 (m, 4H), 1.40 (s, 9H). Mass calculated for (C₂₇H₃₁BrClN₅₀O₄+H)⁺606.1, found 605.9.

### Compound 79: 6-Bromo-N₂-(4-chlorophenyl)-N₃-(2-(4-methylpiperazin-1-yl)ethyl)-1 H-indole-2,3-dicarboxamide

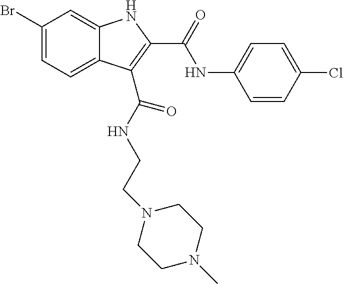 [SMILES: CN1CCN(CCNC(=O)c2c(C(=O)Nc3ccc(Cl)cc3)[nH]c3cc(Br)ccc23)CC1]

Prepared according to general method XXII from 78-iii and 2-(4-methylpiperazin-1-yl)ethan-1-amine (79 mg, 62%). ¹H NMR (400 MHz, DMSO) δ 13.14 (s, 1H), 12.60 (s, 1H), 8.58 (t, J=5.4 Hz, 1H), 8.05 (d, J=8.8 Hz, 1H), 7.82-7.70 (m, 3H), 7.48 (d, J=8.8 Hz, 2H), 7.37 (dd, J=8.8, 1.8 Hz, 1H), 3.53 (q, J=6.1 Hz, 2H), 2.58 (t, J=6.3 Hz, 2H), 2.48-2.24 (m, 6H), 2.17 (s, 3H). Mass calculated for (C₂₃H₂₅BrClN₅O₂+H)⁺520.1, found 519.9.

### Compound 80: 6-Bromo-N₂-(4-chlorophenyl)-N₃-(2-(piperazin-1-yl)ethyl)-1H-indole-2,3-dicarboxamide

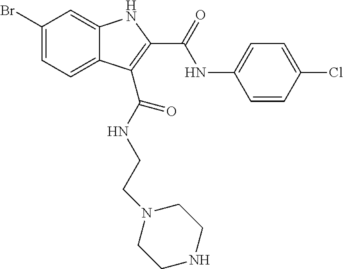 [SMILES: O=C(Nc1ccc(Cl)cc1)c1[nH]c2cc(Br)ccc2c1C(=O)NCCN1CCNCC1]

Prepared according to general method XVI from compound 78 mono-TFA salt (53 mg, 91%). ¹H NMR (400 MHz, DMSO) δ 12.79 (bs, 1H), 8.90 (bs, 1H), 8.11 (d, J=8.8 Hz, 1H), 7.78 (d, J=8.8 Hz, 2H), 7.71 (d, J=1.5 Hz, 1H), 7.47 (d, J=8.8 Hz, 2H), 7.31 (d, J=8.3 Hz, 1H), 3.58-3.46 (m, 2H), 2.76-2.68 (m, 4H), 2.59-2.52 (m, 2H), 2.40 (bs, 4H). Mass calculated for (C₂₂H₂₃BrClN₅O₂+H)⁺ 506.1, found 505.9.

### Synthesis of Intermediates 81-i and 81-ii

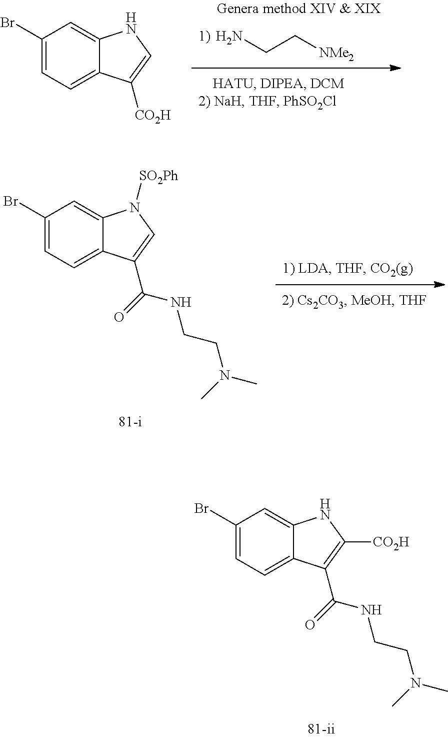

### Intermediate 81-i: 6-Bromo-N-(2-(dimethylamino)ethyl)-1-(phenylsulfonyl)-1H-indole-3-carboxamide

Prepared according to general method XIV and XIX from 6-bromo-1H-indole-3-carboxylic acid (1.2 g, 80%). ¹H NMR (400 MHz, DMSO) δ 8.69 (t, J=5.7 Hz, 1H), 8.55 (s, 1H), 8.12 (d, J=8.6 Hz, 1H), 8.10-8.05 (m, 3H), 7.83-7.77 (m, 1H), 7.70 (t, J=7.8 Hz, 2H), 7.56 (dd, J=8.5, 1.8 Hz, 1H), 3.61 (q, J=5.9 Hz, 2H), 3.29-3.22 (m, 2H), 2.87 (s, 6H).

### Intermediate 81-ii: 6-Bromo-3-((2-(dimethylamino)ethyl)carbamoyl)-1H-indole-2-carboxylic Acid

LDA (2M in THF/heptanes/ethylbenzene, 1.5 mmol) was slowly added to a stirred solution of 81-i (1.0 mmol) in THF (7.5 mL) at −78° C. under Ar. The mixture was then warmed to 0° C. and stirred for 15 min. CO₂ gas was bubbled for 1 h and then the reaction was quenched with H₂O. The mixture was diluted with EtOAc and washed with H₂O, brine, dried over anhydrous Na₂SO₄ and concentrated under reduced pressure. The residue was dissolved in 18 mL of THF:MeOH, (2:1) and Cs₂CO₃ (2.0 mmol) was added and the mixture has heated in a μwave reactor at 90° C. for 30 min. The mixture was diluted with EtOAc and washed with 0.1 M aqueous HCl, brine, dried over anhydrous Na₂SO₄ and concentrated under reduced pressure. The crude intermediate 81-ii was partially purified by silica gel chromatography in MeOH/DCM and used in the next step.

### Synthesis of Compound 81

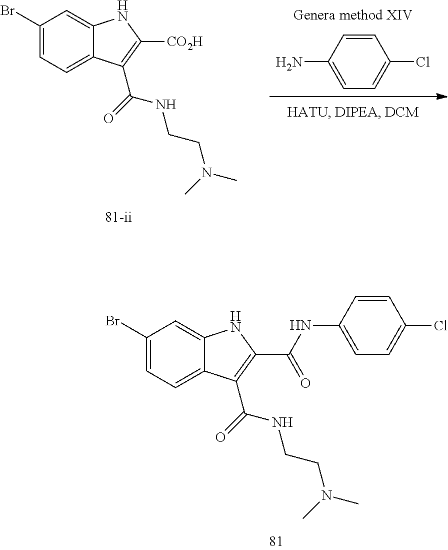 [SMILES: CN(C)CCNC(=O)c1c(C(=O)O)[nH]c2cc(Br)ccc12 ; Nc1ccc(Cl)cc1 ; CN(C)CCNC(=O)c1c(C(=O)Nc2ccc(Cl)cc2)[nH]c2cc(Br)ccc12]

### Compound 81: 6-Bromo-N₂-(4-chlorophenyl)-N₃-(2-(dimethylamino)ethyl)-1H-indole-2,3-dicarboxamide

Prepared according to general method XIV from 81-ii and 4-chloroaniline (16 mg, 17%). ¹H NMR (400 MHz, DMSO) δ 13.11 (s, 1H), 12.58 (s, 1H), 8.59 (t, J=5.4 Hz, 1H), 7.91 (d, J=8.8 Hz, 1H), 7.79-7.72 (m, 3H), 7.49 (d, J=8.8 Hz, 2H), 7.40 (dd, J=8.8, 1.8 Hz, 1H), 3.54 (q, J=6.0 Hz, 2H), 2.61 (bs, 2H), 2.31 (s, 6H). Mass calculated for (C₂₀H₂₀BrClN₄O₂+H)⁺465.0, found 465.1.

### Synthesis of Compound 82

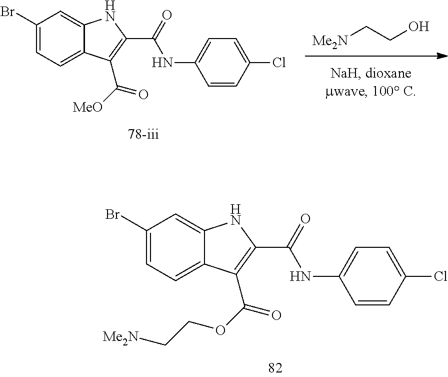 [SMILES: COC(=O)c1c(C(=O)Nc2ccc(Cl)cc2)[nH]c2cc(Br)ccc12 ; CN(C)CCO ; CN(C)CCOC(=O)c1c(C(=O)Nc2ccc(Cl)cc2)[nH]c2cc(Br)ccc12]

### Compound 82: 2-(Dimethylamino)ethyl 6-bromo-2-((4-chlorophenyl)carbamoyl)-1 H-indole-3-carboxylate

To a stirred solution of 2-(dimethylamino)ethan-1-ol (200 uL, 2.0 mmol) in dioxane (2 mL) under Ar was added NaH (60% in oil, 10 mg, 0.25 mmol). The mixture was stirred at rt for 15 min followed by the addition of 78-iii (50 mg, 0.12 mmol). The resulting mixture was heated with microwave at 100° C. for 1 h and then diluted with EtOAc (200 mL). The mixture was washed with H₂O, brine, dried over anhydrous Na₂SO₄, filtered and concentrated under reduced pressure. The crude product was purified by silica gel chromatography, eluting with a MeOH/DCM gradient, to provide compound 82 (21 mg, 37%). ¹H NMR (400 MHz, DMSO) δ 12.95 (s, 1H), 12.37 (s, 1H), 8.13 (d, J=8.7 Hz, 1H), 7.80 (d, J=8.9 Hz, 2H), 7.75 (d, J=1.6 Hz, 1H), 7.50 (d, J=8.8 Hz, 2H), 7.43 (dd, J=8.8, 1.8 Hz, 1H), 4.47 (t, J=5.7 Hz, 2H), 2.66 (t, J=5.7 Hz, 2H), 2.22 (s, 6H). Mass calculated for (C₂₀H₁₉BrClN₃O₃+H)⁺466.0, found 565.8.

### Synthesis of Compound 83

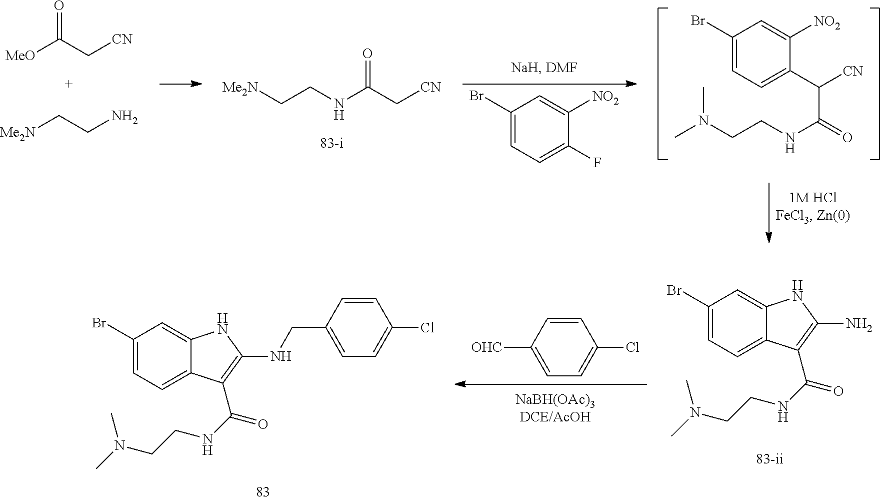

### Intermediate 83-i: 2-Cyano-N-(2-(dimethylamino)ethyl)acetamide

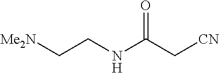 [SMILES: CN(C)CCNC(=O)CC#N]

A solution of methyl 2-cyanoacetate and N¹,N¹-dimethylethane-1,2-diamine was stirred at ambient temperature 48 h then diluted with Et₂O, concentrated in vacuo, co-evaporated with more Et₂O (3×) to afford acetamide 83-i (1.76 g, quantitative). ¹H NMR (400 MHz, DMSO) δ 8.16 (s, 1H), 3.63 (s, 2H), 3.17 (td, J=6.5, 5.5 Hz, 2H), 2.29 (t, J=6.5 Hz, 2H), 2.15 (s, 6H).

### Intermediate 83-ii: 2-Amino-6-bromo-N-(2-(dimethylamino)ethyl)-1H-indole-3-carboxamide

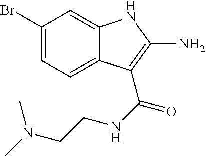 [SMILES: CN(C)CCNC(=O)c1c(N)[nH]c2cc(Br)ccc12]

The 2-aminoindole intermediate 83-ii was prepared according to literature procedures (WO 2011/056739). NaH (1.6 equiv) was added to a stirred solution of acetamide 83-i (1 equiv) in DMF. After 10 minutes, 5-bromo-2-fluoronitrobenzene (0.95 equiv) was added. After 1 h, the reaction mixture was quenched with 1M HCl (2 equiv) followed by the addition of FeCl₃ (3 equiv) and zinc powder (10 equiv). The resulting mixture was stirred at 100° C. for 2 h, cooled down to ambient temperature, passed through a bed of celite and rinsed with EtOAc. The brown filtrate was successively washed with sat. aq. NaHCO₃ (1×), H₂O (3×) and brine (1×) then dried (MgSO₄), filtered and concentrated in vacuo to afford a brown paste. The crude product was purified by silica gel column chromatography, eluted with 5-15% (5% NH₄OH/MeOH) in CH₂Cl₂, to afford the 3-amido-2-aminoindole intermediate 83-ii (278 mg, 9%). ¹H NMR (400 MHz, DMSO) δ 10.67 (s, 1H), 7.42 (d, J=8.4 Hz, 1H), 7.29 (d, J=1.9 Hz, 1H), 7.08 (dd, J=8.4, 1.9 Hz, 1H), 6.86 (s, 2H), 6.67 (t, J=5.5 Hz, 1H), 2.42 (t, J=6.8 Hz, 2H), 2.21 (s, 6H). Mass calculated for (C₁₃H₁₇BrN₄O+H)⁺325.1, found 325.5.

### Compound 83: 6-bromo-2-(4-chlorobenzylamino)-N-(2-(dimethylamino)ethyl)-1H-indole-3-carboxamide

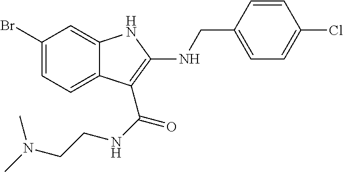 [SMILES: CN(C)CCNC(=O)c1c(NCc2ccc(Cl)cc2)[nH]c2cc(Br)ccc12]

A mixture of 83-ii (1.0 mmol), aldehyde (2.0 mmol), NaBH(OAc)₃ (4.5 mmol), acetic acid (4.0 mmol) in DCE (5 mL) was stirred at ambient temperature for 1-2 days, slowly quenched with a saturated aqueous solution of NaHCO₃ (10 mL), diluted with water (15 mL) and extracted with EtOAc (3×25 mL). The combined organics was dried over MgSO₄, filtered and concentrated in vacuo. The crude product was purified by silica gel chromatography to give the desired product (18 mg, 20%). ¹H NMR (400 MHz, DMSO) δ 11.22 (s, 1H), 8.36 (t, J=6.9 Hz, 1H), 7.46 (d, J=8.4 Hz, 1H), 7.44-7.40 (m, 2H), 7.38 (d, J=8.6 Hz, 2H), 7.24 (d, J=1.9 Hz, 1H), 7.12 (dd, J=8.4, 1.9 Hz, 1H), 6.72 (t, J=5.6 Hz, 1H), 4.57 (d, J=6.8 Hz, 2H), 3.35 (m, 2H), 2.41 (t, J=6.9 Hz, 2H), 2.20 (s, 6H). Mass calculated for (C₂₀H₂₂BrClN₄O+H)⁺449.1, found 449.4.

### Synthesis of Compound 84

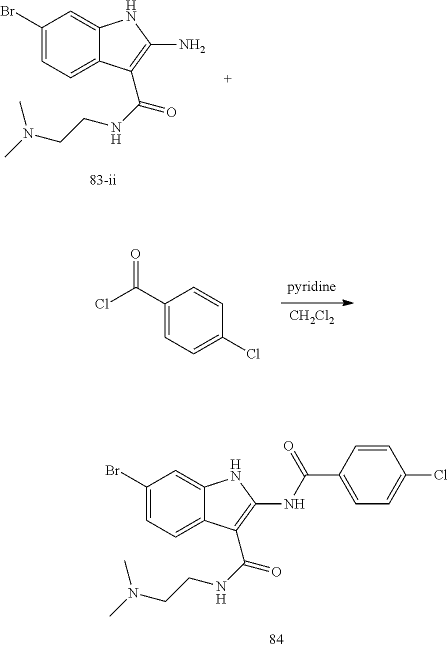 [SMILES: CN(C)CCNC(=O)c1c(N)[nH]c2cc(Br)ccc12 ; O=C(Cl)c1ccc(Cl)cc1 ; "CH2Cl2" ; CN(C)CCNC(=O)c1c(NC(=O)c2ccc(Cl)cc2)[nH]c2cc(Br)ccc12]

### Compound 84: 6-Bromo-2-(4-chlorobenzamido)-N-(2-(dimethylamino)ethyl)-1H-indole-3-carboxamide

Pyridine (10 equiv) and 4-chlorobenzoyl chloride (4 equiv) were successively added to a stirring suspension of 83-ii (1 equiv) in CH₂Cl₂. After stirring for 16-24 h at ambient temperature, the reaction mixture was concentrated in vacuo and purified by preparative HPLC (ACN/H₂O with 0.1% formic acid) to afford the anilide product. ¹H NMR (400 MHz, DMSO) δ 12.70 (s, 1H), 12.44 (s, 1H), 9.33 (s, 1H), 7.99 (d, J=8.6 Hz, 2H), 7.93 (d, J=8.6 Hz, 1H), 7.83 (d, J=1.9 Hz, 1H), 7.80-7.76 (m, 2H), 7.37 (dd, J=8.5, 1.9 Hz, 1H), 6.54 (s, 1H), 3.73 (q, J=5.9 Hz, 2H), 2.89 (d, J=3.5 Hz, 6H). Mass calculated for (C₂₀H₂₀BrClN₄O₂+H)⁺463.1, found 463.0.

### General Method XXIII

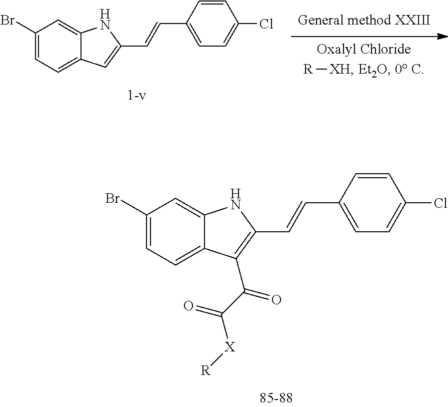

To a solution of 1-v (1 eq.) in diethyl ether (0.1 M) at 0° C. was added dropwise oxalyl chloride (1.1 eq.), and the solution was stirred at 0° C. for 1.5 hours. Progress of the acylation was monitored by quenching a small sample in methanol and analyzing by LCMS. Where X=O, the alcohol or water (˜⅓ volume with respect to solvent) was added followed by Hunig's base (3 eq.). Where X=NH, a 2 M solution of the corresponding amine (6.67 eq.) in THF was added with no exogenous base. The solution was then stirred for 3 h at 0° C. Upon completion, the reaction mixture was diluted EtOAc, washed with either 1 M HCl (X=O) or 1 M NaOH (X=NH), followed by H₂O and brine. The organic phase was then dried over Na₂SO₄, filtered and concentrated in vacuo. Purification via preparative HPLC (ACN/H₂O with 0.1% formic acid) yielded pure products.

### Compound 85: (E)-2-(6-bromo-2-(4-chlorostyryl)-1H-indol-3-yl)-2-oxoacetic Acid

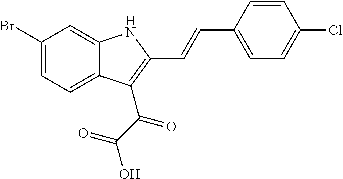 [SMILES: O=C(O)C(=O)c1c(/C=C/c2ccc(Cl)cc2)[nH]c2cc(Br)ccc12]

Prepared according to general method XXIII from 1-v (8.9 mg, 46%). ¹H NMR (400 MHz, DMSO-d₆) δ 12.76 (s, 1H), 7.85 (d, J=8.6 Hz, 1H), 7.71 (d, J=16.5 Hz, 1H), 7.66 (d, J=1.8 Hz, 1H), 7.65-7.51 (m, 5H), 7.39 (dd, J=8.6, 1.8 Hz, 1H). Mass calculated for (C₁₈H₁₁BrClNO₃−H)⁻ 403.6, found 403.8.

### Compound 86: (E)-Methyl 2-(6-bromo-2-(4-chlorostyryl)-1H-indol-3-yl)-2-oxoacetate

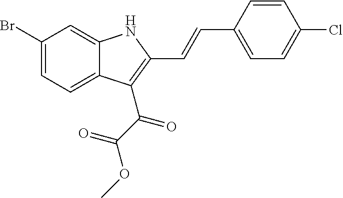 [SMILES: COC(=O)C(=O)c1c(/C=C/c2ccc(Cl)cc2)[nH]c2cc(Br)ccc12]

Prepared according to general method XXIII from 1-v and methanol (15.5 mg, 37%). ¹H NMR (400 MHz, DMSO-d₆) δ 12.89 (s, 1H), 7.71-7.66 (m, 2H), 7.64 (d, J=8.6 Hz, 2H), 7.59 (s, 2H), 7.55 (d, J=8.6 Hz, 2H), 7.40 (dd, J=8.6, 1.7 Hz, 1H), 3.96 (s, 3H). Mass calculated for (C₁₉H₁₃BrClNO₃−H)⁻ 418.0, found 418.0.

### Compound 87: (E)-2-(6-bromo-2-(4-chlorostyryl)-1H-indol-3-yl)-N-methyl-2-oxoacetamide

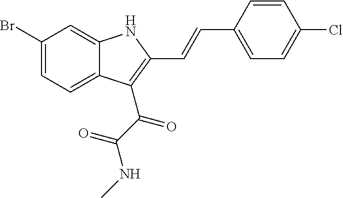 [SMILES: CNC(=O)C(=O)c1c(/C=C/c2ccc(Cl)cc2)[nH]c2cc(Br)ccc12]

Prepared according to general method XXIII from 1-v and methylamine (10.2 mg, 24%). ¹H NMR (400 MHz, DMSO-d₆) δ 12.62 (s, 1H), 8.82 (q, J=4.5 Hz, 1H), 7.83 (d, J=8.6 Hz, 1H), 7.70 (d, J=16.6 Hz, 1H), 7.63 (d, J=1.8 Hz, 1H), 7.59 (d, J=8.7 Hz, 2H), 7.56 (d, J=6.1 Hz, 2H), 7.52 (d, J=13.9 Hz, 1H), 7.35 (dd, J=8.6, 1.8 Hz, 1H), 2.82 (d, J=4.7 Hz, 3H). Mass calculated for (C₁₉H₁₄BrClN₂O₂−H)⁻ 417.0, found 417.0.

### Compound 88: (E)-2-(6-bromo-2-(4-chlorostyryl)-1H-indol-3-yl)-2-oxo-N-(2-(pyridin-2-yl)ethyl)acetamide

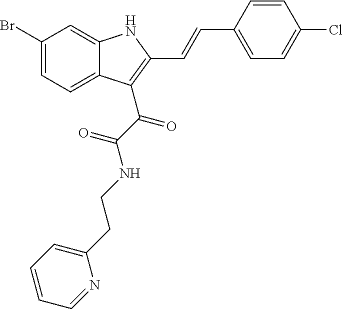 [SMILES: O=C(NCCc1ccccn1)C(=O)c1c(/C=C/c2ccc(Cl)cc2)[nH]c2cc(Br)ccc12]

Prepared according to general method XXIII from 1-v and 2-(pyridin-2-yl)ethan-1-amine (10.7 mg, 7%). ¹H NMR (400 MHz, Acetone-d₆) δ 11.67 (s, 1H), 8.75-8.65 (m, 1H), 8.35 (s, 1H), 8.07 (td, J=7.7, 1.8 Hz, 1H), 7.95 (d, J=5.1 Hz, 1H), 7.92 (d, J=3.0 Hz, 1H), 7.70-7.60 (m, 3H), 7.58-7.52 (m, 1H), 7.52-7.45 (m, 3H), 7.32 (dd, J=8.6, 1.8 Hz, 1H), 7.30-7.22 (m, 1H), 3.98-3.89 (m, 2H), 3.33 (t, J=6.8 Hz, 2H). Mass calculated for (C₂₅H₁₉BrClN₃O₂−H)⁻ 508.0, found 508.0.

### Compound 89: 2-(6-bromo-2-(4-chlorophenethyl)-1H-indol-3-yl)-N-methyl-2-oxoacetamide

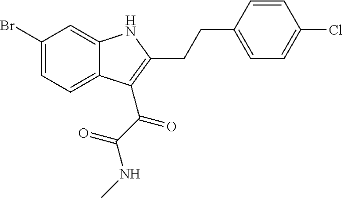 [SMILES: CNC(=O)C(=O)c1c(CCc2ccc(Cl)cc2)[nH]c2cc(Br)ccc12]

Prepared according to general method IV from 87 (56.5 mg, 38%). ¹H NMR (400 MHz, DMSO-d₆) δ 12.30 (s, 1H), 8.73 (q, J=4.3 Hz, 1H), 7.86 (d, J=8.6 Hz, 1H), 7.62 (d, J=1.8 Hz, 1H), 7.39 (d, J=8.3 Hz, 2H), 7.33 (dd, J=8.6, 1.8 Hz, 1H), 7.28 (d, J=8.4 Hz, 2H), 3.20 (dd, J=10.0, 6.4 Hz, 2H), 2.97 (dd, J=10.1, 6.2 Hz, 2H), 2.77 (d, J=4.7 Hz, 3H). Mass calculated for (C₁₉H₁₆BrClN₂O₂−H)⁻ 419.0, found 419.0.

### Compound 90: (E)-2-(6-bromo-2-(4-chlorostyryl)-1H-indol-3-yl)-2-hydroxy-N-methylacetamide

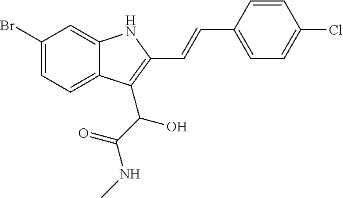 [SMILES: CNC(=O)C(O)c1c(/C=C/c2ccc(Cl)cc2)[nH]c2cc(Br)ccc12]

To a solution of compound 87 (132.5 mg, 0.32 mmol) in MeOH/DCM (0.1 M) at 0° C. was added sodium borohydride (14.5 mg, 0.38 mmol, 1.2 eq.), and the solution was stirred for 3 h. Upon reaction completion, the product was filtered out, washed with water, and dried in vacuo to yield compound 90 (103.5 mg, 78%) as a yellow solid. ¹H NMR (400 MHz, DMSO-d₆) δ 11.47 (s, 1H), 8.17 (q, J=4.6 Hz, 1H), 7.66 (d, J=8.4 Hz, 2H), 7.57 (d, J=8.4 Hz, 1H), 7.51 (d, J=16.5 Hz, 1H), 7.49-7.43 (m, 3H), 7.18 (d, J=16.4 Hz, 1H), 7.09 (dd, J=8.5, 1.8 Hz, 1H), 5.96 (d, J=3.7 Hz, 1H), 5.45 (d, J=3.8 Hz, 1H), 2.66 (d, J=4.7 Hz, 3H). Mass calculated for (C₁₉H₁₆⁷⁹Br³⁵ClN₂O₂−H)⁻ 417.0, found 417.0.

### Compound 91: 2-(6-Bromo-2-(4-chlorophenethyl)-1H-indol-3-yl)-N-methylacetamide

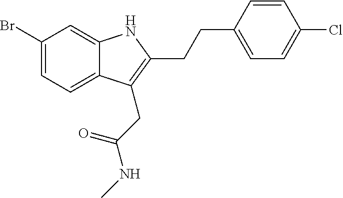 [SMILES: CNC(=O)Cc1c(CCc2ccc(Cl)cc2)[nH]c2cc(Br)ccc12]

Prepared according to general method IV from compound 90 (4.9 mg, 10%). H NMR (400 MHz, DMSO-d₆) δ 11.06 (s, 1H), 7.68 (q, J=4.5 Hz, 1H), 7.48-7.38 (m, 2H), 7.33 (d, J=8.4 Hz, 2H), 7.25 (d, J=8.3 Hz, 2H), 7.06 (dd, J=8.4, 1.8 Hz, 1H), 3.06-2.85 (m, 4H), 2.54 (s, 3H). Mass calculated for (C₁₉H₁₈BrClN₂O−H)⁻ 405.0, found 405.1.

### General Method XXIV

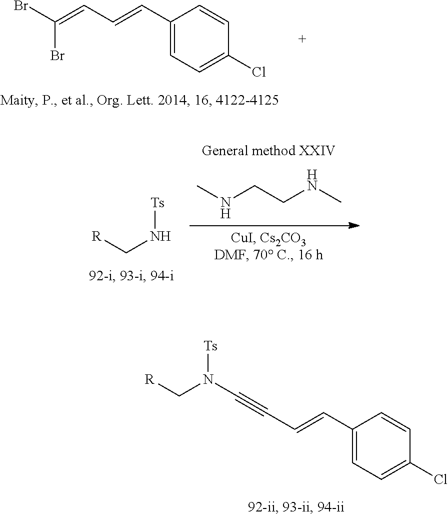

(E)-1-Chloro-4-(4,4-dibromobuta-1,3-dienyl)benzene (prepared according to Maity, P., et al. Org. Lett. 2014, 16 4122-4125), the appropriate tosylamine (1.05 eq), and Cs₂CO₃ (4.0 eq) were combined and taken up in DMF (0.33 M) under N₂. Next, N,N′-dimethylethylenediamine (0.18 eq) and CuI (0.12 eq) were added and the reaction was heated to 70° C. The reaction was monitored by TLC and/or HPLC (product had UV response only). After 3-5 h, the reaction was cooled to rt, water was added, and the mixture was extracted four times with diethyl ether. The combined organics were washed with water, brine, dried over sodium sulfate, filtered and concentrated under reduced pressure. The crude material was purified by silica gel chromatography, eluting with an EtOAc/hexanes gradient, to yield the desired adduct.

### Intermediate 92-ii: (E)-N-benzyl-N-(4-(4-chlorophenyl)but-3-en-1-ynyl)-4-methylbenzenesulfonamide

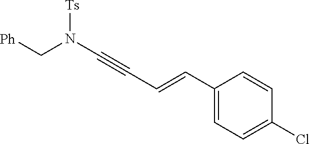 [SMILES: Cc1ccc(S(=O)(=O)N(C#C/C=C/c2ccc(Cl)cc2)Cc2ccccc2)cc1]

Prepared according to general method XXIV from 92-i (780 mg, 86%). ¹H NMR (400 MHz, Chloroform-d) δ 7.79 (d, J=8.3 Hz, 2H), 7.41-7.21 (m, 11H), 6.66 (d, J=16.1 Hz, 1H), 6.15 (d, J=16.2 Hz, 1H), 4.58 (s, 2H), 2.48 (s, 3H).

### Intermediate 93-ii: (E)-N-(4-(4-chlorophenyl)but-3-en-1-ynyl)-N-ethyl-4-methylbenzenesulfonamide

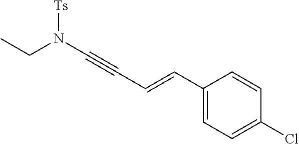 [SMILES: CCN(C#C/C=C/c1ccc(Cl)cc1)S(=O)(=O)c1ccc(C)cc1]

Prepared according to general method XXIV from 93-i (254 mg, 81%). ¹H NMR (400 MHz, Chloroform-d) δ 7.84 (d, J=8.3 Hz, 2H), 7.38 (d, J=8.1 Hz, 2H), 7.31 (s, 4H), 6.79 (d, J=16.2 Hz, 1H), 6.25 (d, J=16.2 Hz, 1H), 3.48 (q, J=7.2 Hz, 2H), 2.48 (s, 3H), 1.27 (t, J=7.2 Hz, 3H).

### Intermediate 94-ii: (E)-N-(4-(4-chlorophenyl)but-3-en-1-ynyl)-N-isobutyl-4-methylbenzenesulfonamide

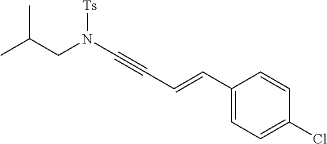 [SMILES: Cc1ccc(S(=O)(=O)N(C#C/C=C/c2ccc(Cl)cc2)CC(C)C)cc1]

Prepared according to general method XXIV from 94-i (144 mg, 43%). ¹H NMR (400 MHz, Chloroform-d) δ 7.84 (d, J=8.3 Hz, 2H), 7.38 (d, J=8.1 Hz, 2H), 7.31 (s, 4H), 6.78 (d, J=16.1 Hz, 1H), 6.24 (d, J=16.2 Hz, 1H), 3.16 (d, J=7.4 Hz, 2H), 2.48 (s, 3H), 2.08 (hept, J=6.9 Hz, 1H), 0.98 (d, J=6.7 Hz, 6H).

### a) General Method XXV

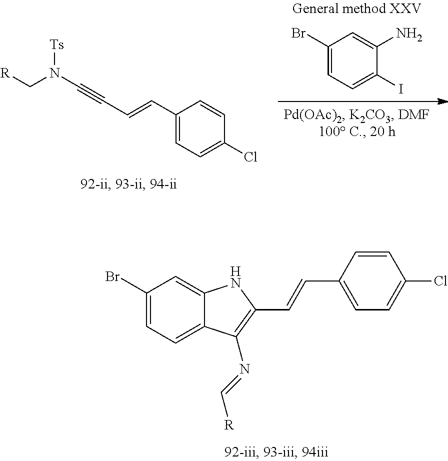

The appropriate tosylamine intermediate (92-ii-94-ii) was taken up in DMF (0.33 M) under N₂ and 5-bromo-2-iodoaniline (1.05 eq) was added. Next, K₂CO₃ (4.0 eq) and Pd(OAc)₂ (0.05 eq) were added and the reaction was heated to 100° C. When complete, water was added, and the mixture was extracted four time with EtOAc, combined organics washed with saturated NaHCO₃, brine, dried over sodium sulfate, filtered and concentrated under reduced pressure. The crude product was purified by silica gel chromatography, eluting with an EtOAc/hexanes gradient, to yield the desired intermediate.

### Intermediate 92-iii: (E)-N-Benzylidene-6-bromo-2-(4-chlorostyryl)-1H-indol-3-amine (96iii)

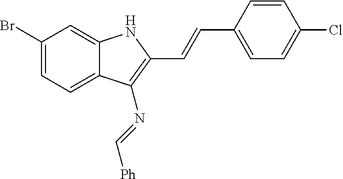 [SMILES: Clc1ccc(/C=C/c2[nH]c3cc(Br)ccc3c2/N=C/c2ccccc2)cc1]

Prepared according to general method XXV from 92-ii (157 mg, 32%). ¹H NMR (400 MHz, Chloroform-d) δ 9.02 (s, 1H), 8.20 (s, 1H), 8.05-7.95 (m, 1H), 7.80 (d, J=8.5 Hz, 1H), 7.62 (d, J=16.7 Hz, 1H), 7.58-7.43 (m, 8H), 7.38 (d, J=8.5 Hz, 2H), 6.93 (d, J=16.7 Hz, 1H).

### Synthesis of Compound 92

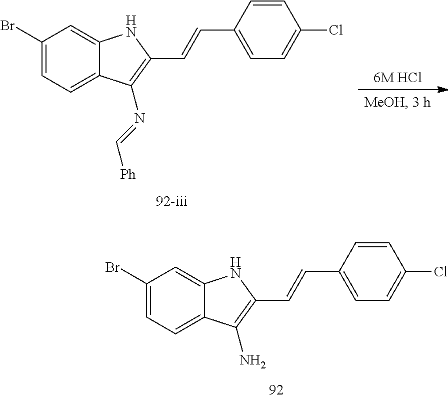 [SMILES: Clc1ccc(/C=C/c2[nH]c3cc(Br)ccc3c2/N=C/c2ccccc2)cc1 ; "6M HCl" ; "MeOH, 3 h" ; Nc1c(/C=C/c2ccc(Cl)cc2)[nH]c2cc(Br)ccc12]

### Compound 92: (E)-6-bromo-2-(4-chlorostyryl)-1H-indol-3-amine

Intermediate 92-iii was taken up in MeOH (10 mL) and treated with 6 M HCl (2 mL). After 30 min, no starting material was visible by HPLC-MS. After 1 h, pH was adjusted to >12 with 5 M NaOH. Organic solvent was removed under reduced pressure, then water was added and the solution was extracted 3×30 mL DCM, washed with brine, dried over sodium sulfate, filtered and concentrated under reduced pressure. The crude residue was purified by silica gel chromatography, eluting with an EtOAc/hexanes gradient, to provide compound 92 (50 mg, 40%). ¹H NMR (400 MHz, Chloroform-d) δ 7.61 (s, 1H), 7.43 (d, J=8.5 Hz, 2H), 7.38-7.30 (m, 3H), 7.19 (dd, J=8.4, 1.7 Hz, 1H), 7.12 (d, J=16.3 Hz, 1H), 6.64 (d, J=16.3 Hz, 1H). Mass calculated for (C₂₄H₂₂⁷⁹Br³⁵ClN₂+H)⁺347.0, found 347.0.

### b) General Method XXVI

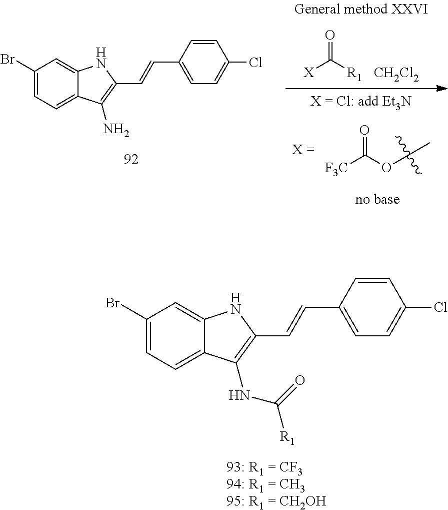

Compound 92 was taken up in DCM (0.1 M) and the appropriate acylating agent was added (1.2-1.5 eq) along with optional triethylamine (2.0 eq). Upon completion of the reactions (TLC and/or HPLC-MS), they were concentrated under reduced pressure and purified directly by flash chromatography, eluting with an EtOAc/hexanes gradient, and/or preparative HPLC (ACN/H₂O with 0.1% formic acid).

### Compound 93: (E)-N-(6-bromo-2-(4-chlorostyryl)-1H-indol-3-yl)-2,2,2-trifluoroacetamide

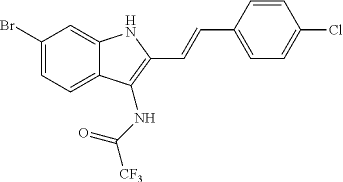 [SMILES: O=C(Nc1c(/C=C/c2ccc(Cl)cc2)[nH]c2cc(Br)ccc12)C(F)(F)F]

Prepared according to general method XXVI with trifluoroacetic anhydride (3.9 mg, 39%). ¹H NMR (400 MHz, Chloroform-d) δ 10.89 (s, 1H), 10.19 (s, 1H), 7.61-7.53 (m, 3H), 7.41 (d, J=8.5 Hz, 2H), 7.38 (d, J=8.5 Hz, 1H), 7.32 (d, J=16.6 Hz, 1H), 7.25-7.17 (m, 2H). Mass calculated for (C₁₈H₁₁BrClF₃N₂O−H) 443.0, found 442.9.

### Compound 94: (E)-N-(6-bromo-2-(4-chlorostyryl)-1H-indol-3-yl)acetamide

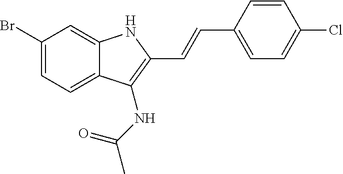 [SMILES: CC(=O)Nc1c(/C=C/c2ccc(Cl)cc2)[nH]c2cc(Br)ccc12]

Prepared according to general method XXVI with acetyl chloride (1.5 eq) (20 mg, 70%). ¹H NMR (400 MHz, DMSO-d₆) δ 11.46 (s, 1H), 9.60 (s, 1H), 7.58 (d, J=8.6 Hz, 2H), 7.53-7.40 (m, 3H), 7.31 (d, J=8.5 Hz, 1H), 7.22 (d, J=16.6 Hz, 1H), 7.16-7.03 (m, 2H), 2.13 (s, 3H). Mass calculated for (C₁₈H₁₄⁷⁹Br³⁵ClN₂O+H)⁺389.0, found 389.0.

### Compound 95: (E)-N-(6-bromo-2-(4-chlorostyryl)-1H-indol-3-yl)-2-hydroxyacetamide

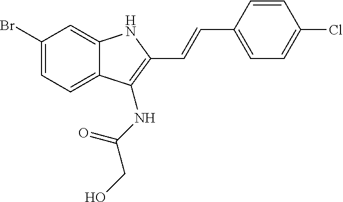 [SMILES: O=C(CO)Nc1c(/C=C/c2ccc(Cl)cc2)[nH]c2cc(Br)ccc12]

Prepared according to general method XXVI with acetoxyacetyl chloride (1.5 eq) to give 20 mg (61%) of a crude acetate that spontaneously saponified in MeOH to give the title compound after prep-HPLC (ACN/H₂O with 0.1% formic acid). ¹H NMR (400 MHz, Chloroform-d) δ 9.50 (s, 1H), 8.42 (s, 1H), 7.41-7.19 (m, 6H), 7.14-7.07 (m, 1H), 7.02 (d, J=16.4 Hz, 1H), 6.87 (d, J=16.5 Hz, 1H), 4.38 (s, 2H). Mass calculated for (C₁₈H₁₄BrClN₂O₂+H)⁺407.0, found 407.0.

### Compound 96: N-(6-Bromo-2-(4-chlorophenethyl)-1H-indol-3-yl)acetamide

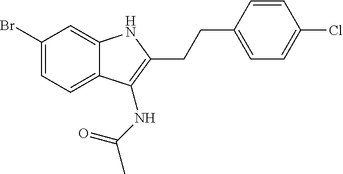 [SMILES: CC(=O)Nc1c(CCc2ccc(Cl)cc2)[nH]c2cc(Br)ccc12]

Prepared according to general method IV from compound 94 (2.4 mg, 42%). ¹H NMR (400 MHz, Chloroform-d) δ 7.93 (s, 1H), 7.46 (s, 1H), 7.39 (s, 1H), 7.29-7.24 (m, 1H), 7.10-7.01 (m, 4H), 6.36 (s, 1H), 3.05 (t, J=6.9 Hz, 2H), 2.97 (t, J=7.0 Hz, 2H), 2.20 (s, 3H). Mass calculated for (C₁₈H₁₆BrClN₂O+H)⁺393.0, found 393.0.

### Compound 97: 2-(6-Bromo-2-(4-chlorophenethyl)-1H-indol-3-ylamino)-2-oxoethyl Acetate

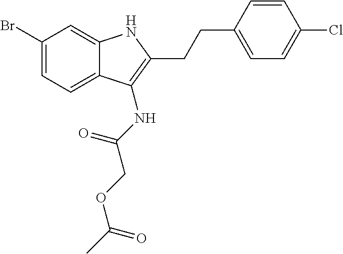 [SMILES: CC(=O)OCC(=O)Nc1c(CCc2ccc(Cl)cc2)[nH]c2cc(Br)ccc12]

Prepared according to general method IV from the crude material of compound 95 (1.6 mg, 21%). ¹H NMR (400 MHz, Chloroform-d) δ 7.87 (s, 1H), 7.41 (s, 1H), 7.28-7.22 (m, 3H), 7.09-7.02 (m, 3H), 4.76 (s, 2H), 2.95 (s, 4H), 2.29 (s, 3H). Mass calculated for (C₂₀H₁₈BrClN₂O₃+H)⁺ 451.0, found 451.0.

### Compound 98: N-benzyl-6-bromo-2-(4-chlorophenethyl)-N-methyl-1H-indol-3-amine

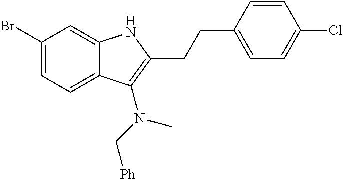 [SMILES: CN(Cc1ccccc1)c1c(CCc2ccc(Cl)cc2)[nH]c2cc(Br)ccc12]

(E)-N-benzylidene-6-bromo-2-(4-chlorostyryl)-1H-indol-3-amine (92-iii) was subjected to general procedure IV, then the resulting crude product was taken up in MeOH (5 mL), purged with N₂, 5% Pt/C (17.6 mg, 0.009 mmol, 0.1 eq) was added, then 4.4% formic acid in MeOH (0.22 mL) was added. After 90 min, another 0.22 mL of 4.4% formic acid in MeOH was added. After 3 d, the mixture was filtered over celite, concentrated under reduced pressure, and purified by prep-HPLC (ACN/H₂O with 0.1% formic acid) to yield compound 98 (605 mg, 18%). ¹H NMR (400 MHz, Acetone-d₆) δ 7.83 (d, J=8.5 Hz, 1H), 7.55 (d, J=1.8 Hz, 1H), 7.31-7.18 (m, 10H), 4.58 (s, 2H), 3.17 (s, 3H), 2.99 (dd, J=8.7, 7.3 Hz, 2H), 2.70 (t, J=8.1 Hz, 2H). Mass calculated for (C₂₄H₂₂BrClN₂+H)⁺455.1, found 455.1.

### General Method XXVII

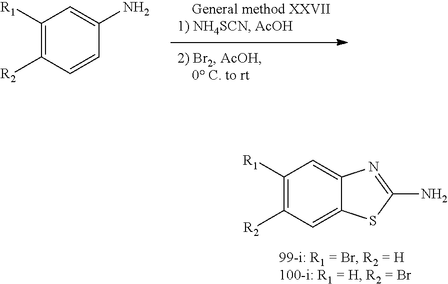

To a solution of the commercially available aniline (1.0 eq) in acetic acid (0.5 M), was added ammonium thiocyanate (5.0 eq) at rt. After 90 min, the thick suspension was cooled to 0° C., and bromine (1.1 eq) in acetic acid (1.0 M) was added. After 1 h, the reaction was allowed to warm to rt. After a subsequent 1 h, the mixture was concentrated to remove acetic acid. A small amount of water was added, then the mixture was adjusted to pH 12 with concentrated ammonium hydroxide. The resulting mixture was extracted with EtOAc (×3) and the combined organics were washed with NaHCO₃₍ₛₐₜ₎, brine, dried over sodium sulfate, filtered and concentrated under reduced pressure. The residue was purified by silica gel chromatography and/or recrystallization from EtOAc/Hex to yield the appropriate 2-aminobenzothiazole.

### Intermediate 99-i: 5-Bromobenzo[d]thiazol-2-amine

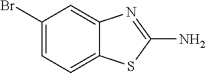 [SMILES: Nc1nc2cc(Br)ccc2s1]

Prepared according to general method XXVII from 3-bromoaniline (1.02 g, 77%). ¹H NMR (400 MHz, DMSO-d₆) δ 7.48 (d, J=8.6 Hz, 1H), 6.98 (d, J=2.4 Hz, 1H), 6.63 (dd, J=8.6, 2.5 Hz, 1H), 6.09 (s, 2H). Mass calculated for (C₇H₅BrN₂S+H)⁺231.0, found 231.4.

### Intermediate 100-i: 6-Bromobenzo[d]thiazol-2-amine

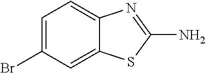 [SMILES: Nc1nc2ccc(Br)cc2s1]

Prepared according to general method XXVII from 4-bromoaniline (740 mg, 56%). ¹H NMR (400 MHz, DMSO-d₆) δ 7.90 (d, J=2.1 Hz, 1H), 7.61 (s, 2H), 7.34 (dd, J=8.5, 2.1 Hz, 1H), 7.25 (d, J=8.5 Hz, 1H). Mass calculated for (C₇H₅BrN₂S+H)⁺231.0, found 231.1.

### c) General Method XXVIII

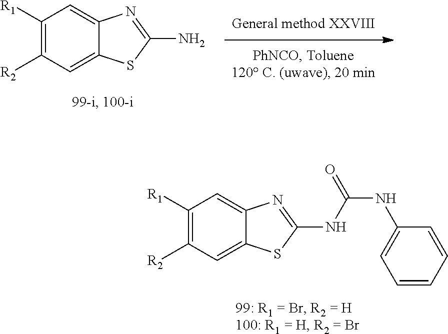

The appropriate 2-aminobenzothiazole was taken up in toluene (0.1-0.2 M) and an isocyanate (1.2 eq) was added. The clear solution was heated to 120° C. in a microwave reactor for 20 min. At the completion of the reaction, a large amount of solid material had crashed out. This solid material was filtered off (washed with DCM if desired). The solid was purified by silica gel chromatography, eluting with an EtOAc/hexanes gradient, to yield the urea product.

### Compound 99: 1-(5-bromobenzo[d]thiazol-2-yl)-3-phenylurea

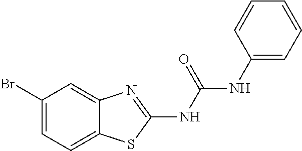 [SMILES: O=C(Nc1ccccc1)Nc1nc2cc(Br)ccc2s1]

Prepared according to general method XXVIII from 99-i and phenylisocyanate (22.8 mg, 52%). ¹H NMR (400 MHz, DMSO-d₆) δ 9.18 (s, 1H), 8.87 (s, 1H), 8.12 (d, J=2.3 Hz, 1H), 7.75 (d, J=8.6 Hz, 1H), 7.50 (dd, J=8.7, 2.4 Hz, 1H), 7.47 (d, J=7.5 Hz, 2H), 7.31 (dd, J=8.5, 7.3 Hz, 2H), 7.02 (tt, J=7.5, 1.2 Hz, 1H). Mass calculated for (C₁₄H₁₀BrN₃OS+H)⁺350.0, found 350.4.

### Compound 100: 1-(6-bromobenzo[d]thiazol-2-yl)-3-phenylurea

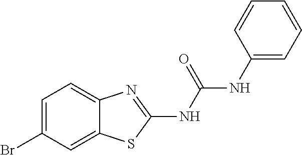 [SMILES: O=C(Nc1ccccc1)Nc1nc2ccc(Br)cc2s1]

Prepared according to general method XXVIII from 100-i and phenylisocyanate (16.9 mg, 15%). ¹H NMR (400 MHz, DMSO-d₆) δ 10.86 (s, 1H), 9.15 (s, 1H), 8.20 (s, 1H), 7.67-7.57 (m, 1H), 7.57-7.47 (m, 3H), 7.35 (t, J=7.8 Hz, 2H), 7.08 (t, J=7.4 Hz, 1H). Mass calculated for (C₁₄H₁₀BrN₃OS+H)⁺350.0, found 350.4.

### Synthesis of Compound 101

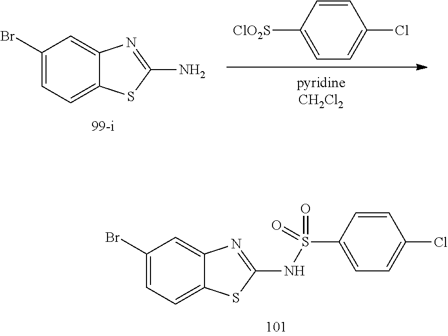 [SMILES: Nc1nc2cc(Br)ccc2s1 ; O=S(=O)(Cl)c1ccc(Cl)cc1 ; O=S(=O)(Nc1nc2cc(Br)ccc2s1)c1ccc(Cl)cc1]

### Compound 101: N-(5-bromobenzo[d]thiazol-2-yl)-4-chlorobenzenesulfonamide

To a solution of intermediate 99-i in DCM (0.5 M) at 0° C. was added chlorobenzenesulfonylchloride (1.2 eq) and pyridine (2.0 eq). The reaction was allowed to warm to rt and stirred for 16 h. The solution was concentrated under reduced pressure and the residue was taken up in saturated NaHCO₃. The resulting mixture was extracted with 2×10 mL EtOAc and the combined organics were washed with saturated NaHCO₃, brine, dried over sodium sulfate, filtered and concentrated under reduced pressure. The crude material could be purified by recrystallization from DCM (35 mg, 36%). ¹H NMR (400 MHz, DMSO-d₆) δ 11.06 (s, 1H), 7.83 (d, J=8.6 Hz, 2H), 7.78-7.61 (m, 3H), 7.47 (d, J=2.4 Hz, 1H), 7.28 (dd, J=8.8, 2.4 Hz, 1H).

### Synthesis of Compound 102

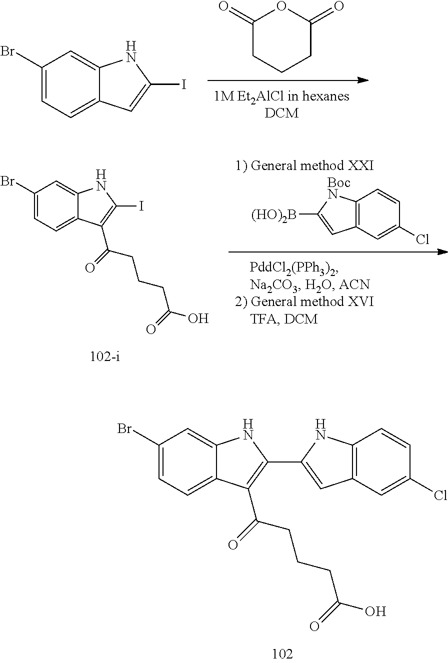 [SMILES: Brc1ccc2cc(I)[nH]c2c1 ; O=C1CCCC(=O)O1 ; O=C(O)CCCC(=O)c1c(I)[nH]c2cc(Br)ccc12 ; CC(C)(C)OC(=O)n1c(B(O)O)cc2cc(Cl)ccc21 ; O=C(O)CCCC(=O)c1c(-c2cc3cc(Cl)ccc3[nH]2)[nH]c2cc(Br)ccc12]

### Intermediate 102-i: 5-(6-bromo-2-iodo-1H-indol-3-yl)-5-oxopentanoic Acid

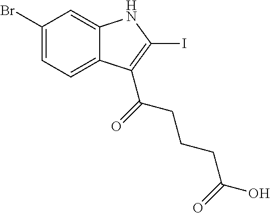 [SMILES: O=C(O)CCCC(=O)c1c(I)[nH]c2cc(Br)ccc12]

To 2-iodo-6-bromoindole (3.2 mmol) in DCM at 0° C. under Ar was added Et₂AlCl (1M in hexanes, 14.8 mmom) and the reaction mixture was stirred at 0° C. for 30 min. Glutaric anhydride (14.8 mmol) was then added in one portion and the reaction mixture was stirred at 0° C. for an additional 5 h. The reaction mixture was quenched with 1M citric acid and the aqueous layer was extracted with EtOAc. The combined organic layers was washed with sat. NaHCO₃. The aqueous layer was acidified with 1M HCl and extracted with EtOAc. The combined EtOAc layers was dried over Na₂SO₄, filtered and concentrated to provide intermediate 102-i (950 mg, yield 69%) which was used in the next step without further purification.

### Compound 102: 5-(6-bromo-5′-chloro-1H,1′H-[2,2′-biindol]-3-yl)-5-oxopentanoic Acid

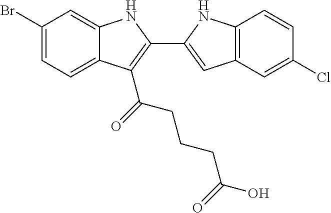 [SMILES: O=C(O)CCCC(=O)c1c(-c2cc3cc(Cl)ccc3[nH]2)[nH]c2cc(Br)ccc12]

Prepared according to general method XXI and XVI from 102-i and (1-(tert-butoxycarbonyl)-5-chloro-1H-indol-2-yl)boronic acid (76 mg, 72%). ¹H NMR (400 MHz, DMSO-d₆) δ 12.57 (s, 1H), 12.43 (d, J=1.9 Hz, 1H), 12.03 (s, 1H), 8.02 (d, J=8.8 Hz, 1H), 7.76 (d, J=2.0 Hz, 1H), 7.70-7.59 (m, 2H), 7.40 (dd, J=8.6, 1.9 Hz, 1H), 7.22 (dd, J=8.7, 2.1 Hz, 1H), 7.17 (dd, J=2.0, 0.9 Hz, 1H), 3.00 (t, J=7.2 Hz, 2H), 2.27 (t, J=7.4 Hz, 2H), 1.89 (p, J=7.3 Hz, 2H). Mass Calculated for (C21H16BrClN2O3)⁻ 457.0, found 457.0.

### General Method XXIX

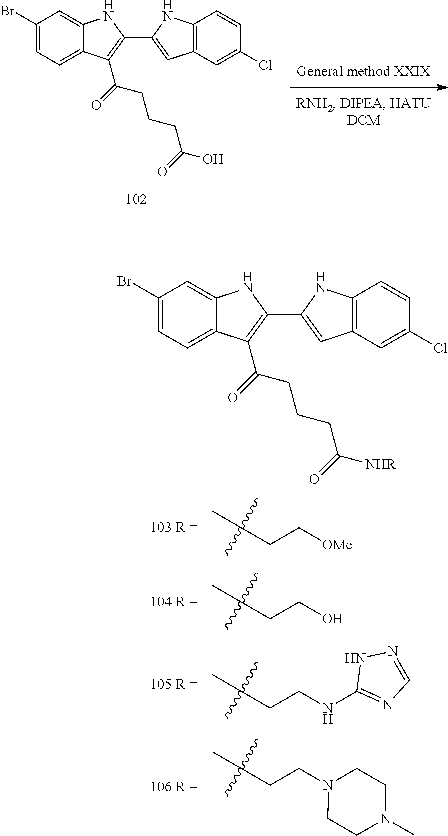

To compound 102 (0.11 mmol) in DCM (5 mL) was added DIPEA (0.33 mmol), the appropriate amine (0.11 mmol), followed by HATU (0.14 mmol). The reaction mixture was stirred at RT overnight. The reaction mixture was concentrated under reduced pressure and the crude material was re-dissolved in EtOAc and the organic layer was washed with sat. NaHCO₃ then with 1M HCl. The combined organic layers was dried over Na₂SO₄, filtered and concentrated under reduced pressure. The crude material was purified via crystallization in EtOAc/hexanes or by silica gel chromatography, eluting with an EtOAc/hexanes gradient, to afford compounds 103-106.

### Compound 103: 5-(6-bromo-5′-chloro-1H,1′H-[2,2′-biindol]-3-yl)-N-(2-methoxyethyl)-5-oxopentanamide

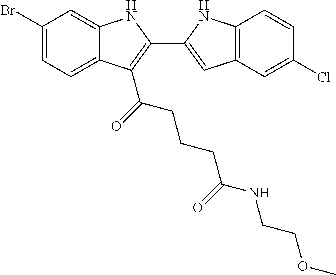 [SMILES: COCCNC(=O)CCCC(=O)c1c(-c2cc3cc(Cl)ccc3[nH]2)[nH]c2cc(Br)ccc12]

Prepared according to general method XXIX from 102 and 2-methoxyethan-1-amine (20 mg, 57%). ¹H NMR (400 MHz, DMSO-d₆) 12.57 (s, 1H), 12.45 (s, 1H), 8.01 (d, J=8.7 Hz, 1H), 7.84 (t, J=5.6 Hz, 1H), 7.76 (d, J=2.1 Hz, 1H), 7.68-7.60 (m, 2H), 7.39 (dd, J=8.7, 1.9 Hz, 1H), 7.22 (dd, J=8.7, 2.1 Hz, 1H), 7.17 (dd, J=2.0, 0.9 Hz, 1H), 3.28 (t, J=5.7 Hz, 2H), 3.20 (s, 3H), 3.15 (q, J=5.7 Hz, 2H), 2.96 (t, J=7.1 Hz, 2H), 2.12 (t, J=7.3 Hz, 2H), 1.89 (p, J=7.0 Hz, 2H). Mass Calculated for (C24H23BrClN3O3)⁻ 514.1, found 514.1.

### Compound 104: 5-(6-bromo-5′-chloro-1H,1′H-[2,2′-biindol]-3-yl)-N-(2-hydroxyethyl)-5-oxopentanamide

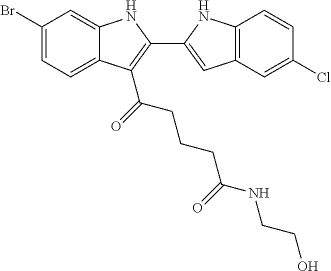 [SMILES: O=C(CCCC(=O)c1c(-c2cc3cc(Cl)ccc3[nH]2)[nH]c2cc(Br)ccc12)NCCO]

Prepared according to general method XXIX from 102 and 2-aminoethan-1-ol (10 mg, 18%). ¹H NMR (400 MHz, DMSO-d₆) δ 12.57 (s, 1H), 12.47 (s, 1H), 8.01 (d, J=8.7 Hz, 1H), 7.82-7.73 (m, 2H), 7.68-7.61 (m, 2H), 7.39 (dd, J=8.6, 1.9 Hz, 1H), 7.22 (dd, J=8.7, 2.1 Hz, 1H), 7.17 (d, J=1.7 Hz, 1H), 4.63 (t, J=5.5 Hz, 1H), 3.07 (q, J=6.0 Hz, 2H), 2.97 (t, J=7.2 Hz, 2H), 2.18-2.06 (m, 2H), 1.89 (p, J=7.3 Hz, 2H). Mass Calculated for (C23H21BrClN3O3)⁻ 500.1, found 500.1.

### Compound 105: 5-(6-bromo-5′-chloro-1H,1′H-[2,2′-biindol]-3-yl)-5-oxo-N-(1H-1,2,4-triazol-5-yl)pentanamide

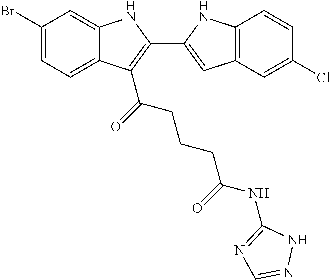 [SMILES: O=C(CCCC(=O)c1c(-c2cc3cc(Cl)ccc3[nH]2)[nH]c2cc(Br)ccc12)Nc1ncn[nH]1]

Prepared according to general method XXIX from 102 and 1H-1,2,4-triazol-5-amine (20 mg, 29%). ¹H NMR (400 MHz, DMSO-d₆) δ 12.62 (s, 1H), 12.46 (d, J=2.1 Hz, 1H), 8.04 (d, J=8.7 Hz, 1H), 7.75 (d, J=2.0 Hz, 1H), 7.69-7.62 (m, 2H), 7.60 (d, J=8.2 Hz, 3H), 7.39 (dd, J=8.6, 1.9 Hz, 1H), 7.25-7.15 (m, 2H), 3.09 (t, J=7.1 Hz, 2H), 3.03 (t, J=7.3 Hz, 2H), 2.04 (p, J=7.2 Hz, 2H). Mass Calculated for (C23H18BrClN6O2)⁻ 523.0, found 523.0.

### Compound 106: 5-(6-bromo-5′-chloro-1H,1′H-[2,2′-biindol]-3-yl)-N-(2-(4-methylpiperazin-1-yl)ethyl)-5-oxopentanamide

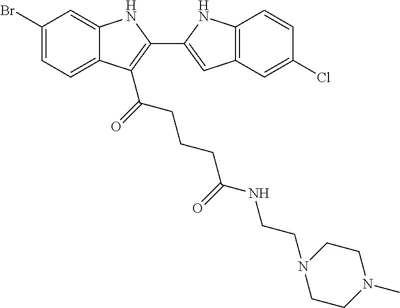 [SMILES: CN1CCN(CCNC(=O)CCCC(=O)c2c(-c3cc4cc(Cl)ccc4[nH]3)[nH]c3cc(Br)ccc23)CC1]

Prepared according to general method XXIX from 102 and 2-(4-methylpiperazin-1-yl)ethan-1-amine (35 mg, 76%). ¹H NMR (400 MHz, DMSO-d₆) δ 12.57 (s, 1H), 12.46 (d, J=2.0 Hz, 1H), 8.01 (d, J=8.7 Hz, 1H), 7.76 (d, J=2.0 Hz, 1H), 7.72-7.57 (m, 3H), 7.39 (dd, J=8.7, 1.9 Hz, 1H), 7.22 (dd, J=8.7, 2.1 Hz, 1H), 7.17 (d, J=1.9 Hz, 1H), 3.08 (q, J=6.5 Hz, 2H), 2.97 (t, J=7.2 Hz, 2H), 2.44-2.19 (m, 10H), 2.19-2.07 (m, 5H), 1.96-1.84 (m, 2H). Mass Calculated for (C28H31 BrClN5O2)⁺584.1, found 584.1.

### Synthesis of Compound 107

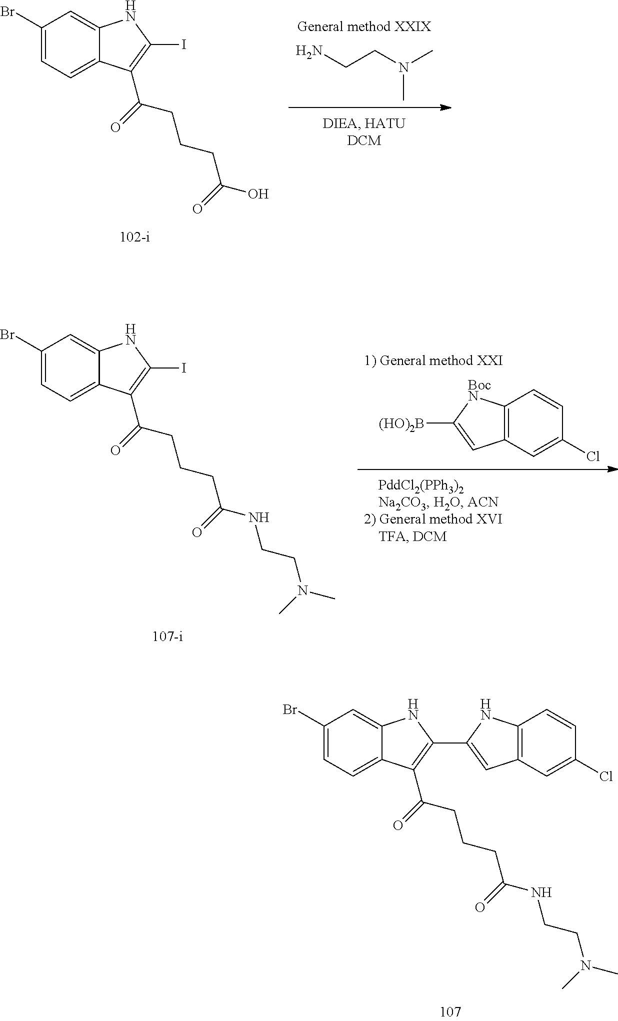

### Intermediate 107-i: 5-(6-bromo-2-iodo-1H-indol-3-yl)-N-(2-(dimethylamino)ethyl)-5-oxopentanamide

Prepared according to general method XXIX from 102-i and N¹,N¹-dimethylethane-1,2-diamine. The crude intermediate 107-i was used in the next step without purification.

### Compound 107: 5-(6-bromo-5′-chloro-1H,1′H-[2,2′-biindol]-3-yl)-N-(2-(dimethylamino)ethyl)-5-oxopentanamide

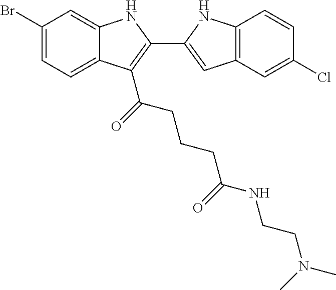 [SMILES: CN(C)CCNC(=O)CCCC(=O)c1c(-c2cc3cc(Cl)ccc3[nH]2)[nH]c2cc(Br)ccc12]

Prepared according to general method XXI and XVI from 107-i and (1-(tert-butoxycarbonyl)-5-chloro-1H-indol-2-yl)boronic acid (28 mg, 23%). ¹H NMR (400 MHz, DMSO-d₆) δ 12.60 (s, 1H), 12.46 (d, J=2.1 Hz, 1H), 8.17 (s, OH), 8.01 (d, J=8.7 Hz, 1H), 7.82 (t, J=5.6 Hz, 1H), 7.76 (d, J=2.0 Hz, 1H), 7.66 (d, J=1.8 Hz, 1H), 7.63 (d, J=8.7 Hz, 1H), 7.39 (dd, J=8.7, 1.9 Hz, 1H), 7.22 (dd, J=8.7, 2.1 Hz, 1H), 7.17 (d, J=1.8 Hz, 1H), 3.17 (q, J=6.3 Hz, 2H), 2.96 (t, J=7.1 Hz, 2H), 2.55 (t, J=6.6 Hz, 2H), 2.36 (s, 6H), 2.13 (t, J=7.4 Hz, 2H), 1.89 (t, J=7.2 Hz, 2H). Mass Calculated for (C25H26BrClN4O2)⁺529.1, found 529.0.

### Synthesis of Compound 108

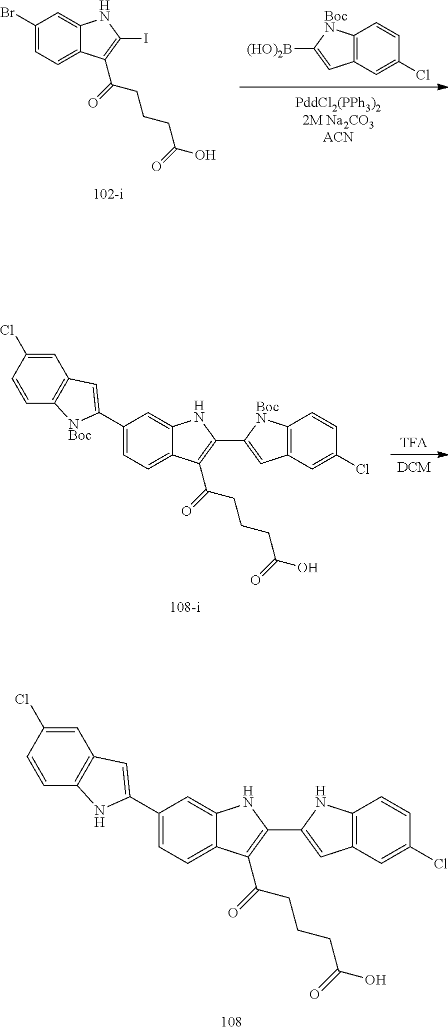

### Intermediate 108-i: 5-(1,1″-bis(tert-butoxycarbonyl)-5,5″-dichloro-1H,1′H,1″H-[2,2′:6′,2″-terindol]-3′-yl)-5-oxopentanoic Acid

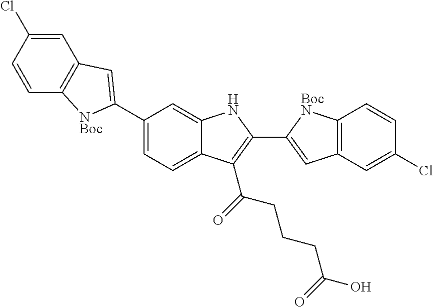 [SMILES: CC(C)(C)OC(=O)n1c(-c2ccc3c(C(=O)CCCC(=O)O)c(-c4cc5cc(Cl)ccc5n4C(=O)OC(C)(C)C)[nH]c3c2)cc2cc(Cl)ccc21]

A mixture of iodide 102-i (0.15 mmol), (1-(tert-butoxycarbonyl)-5-chloro-1H-indol-2-yl)boronic acid (0.21 mmol), PdCl₂(PPh₃)₂ (12 mol %), and 2M Na₂CO₃ (0.37 mL) in ACN (1.5 mL) under Ar was microwaved at 90° C. for 3 h. The reaction was quenched with H₂O and washed with EtOAc. The organic layer was dried over Na₂SO₄, filtered and concentrated under reduced pressure to give 60 mg of compound 108-i which was used in the next step without further purification.

### Compound 108: 5-(5,5″-dichloro-1H,1′H,1″H-[2,2′:6′,2″-terindol]-3′-yl)-5-oxopentanoic Acid

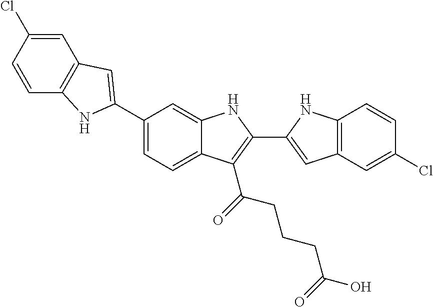 [SMILES: O=C(O)CCCC(=O)c1c(-c2cc3cc(Cl)ccc3[nH]2)[nH]c2cc(-c3cc4cc(Cl)ccc4[nH]3)ccc12]

To trisindole 108-i (0.21 mmol) in DCM (3 mL) was added TFA (1.5 mL). The reaction mixture was stirred at RT for 1 h 30 min. The reaction mixture was concentrated under reduced pressure and the crude material was purified via preparative HPLC (ACN/H₂O with 0.1% formic acid) to give 4 mg (4% yield) of compound 108. ¹H NMR (400 MHz, DMSO-d₆) δ 12.63 (broad s, 2H), 11.87 (d, J=2.2 Hz, 1H), 8.13 (d, J=8.6 Hz, 1H), 7.95 (d, J=1.6 Hz, 1H), 7.78 (dd, J=7.4, 1.8 Hz, 2H), 7.66 (d, J=8.7 Hz, 1H), 7.59 (d, J=2.0 Hz, 1H), 7.44 (d, J=8.6 Hz, 1H), 7.26 (s, 1H), 7.22 (dd, J=8.7, 2.1 Hz, 1H), 7.10 (dd, J=8.6, 2.1 Hz, 1H), 6.92 (d, J=2.0 Hz, 1H), 3.09 (t, J=7.1 Hz, 2H), 2.31 (t, J=7.4 Hz, 2H), 1.94 (p, J=7.3 Hz, 2H). Mass Calculated for (C29H21Cl2N3O3)⁻ 528.1, found 528.1.

### Compound 109: N-(6-bromobenzo[d]thiazol-2-yl)-2-(4-chlorophenyl)acetamide

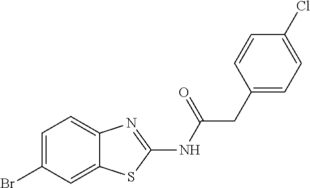 [SMILES: O=C(Cc1ccc(Cl)cc1)Nc1nc2ccc(Br)cc2s1]

Prepared according to general method XIV from 100-i and 4-chlorophenylacetic acid in DCM/DMF (65 mg, 52%). ¹H NMR (400 MHz, DMSO-d₆) δ 12.71 (s, 1H), 8.26 (d, J=2.0 Hz, 1H), 7.69 (d, J=8.6 Hz, 1H), 7.58 (dd, J=8.6, 2.1 Hz, 1H), 7.42 (d, J=8.6 Hz, 2H), 7.38 (d, J=8.6 Hz, 2H), 3.87 (s, 2H). Mass calculated for (C₁₅H₁₀⁷⁹Br³⁵ClN₂OS+H)⁺381.0, found 381.4.

### General Method XXX

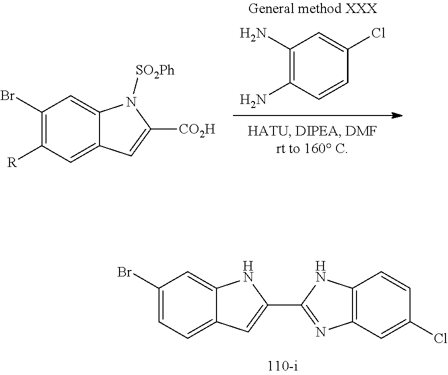

A mixture of the corresponding carboxylic acid (0.16 mmol), 1,2-diamine (0.35 mmol), HATU (0.24 mmol) and DIPEA (0.57 mmol) in DMF (2 mL) was stirred at rt for 3 h and then heated by microwave at 160° C. for 5 h. The reaction mixture was diluted with EtOAc (50 mL) and washed with H₂O (2×20 mL) and brine (20 mL). The organic phase was dried over anhydrous Na₂SO₄, filtered and concentrated under reduced pressure. The crude product was purified by silica gel chromatography, eluting with a MeOH/DCM gradient, to provide the desired adduct.

### Intermediate 110-i: 2-(6-Bromo-1H-indol-2-yl)-5-chloro-1H-benzo[d]imidazole

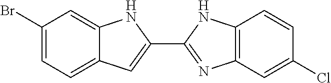 [SMILES: Clc1ccc2[nH]c(-c3cc4ccc(Br)cc4[nH]3)nc2c1]

Prepared according to general method XXX from 6-bromo-1-(phenylsulfonyl)-1H-indole-2-carboxylic acid and 4-chlorobenzene-1,2-diamine (27 mg, 49%). ¹H NMR (400 MHz, DMSO-d₆) b 13.24 (d, J=15.6 Hz, 1H), 12.19 (d, J=12.2 Hz, 1H), 7.92-7.52 (m, 4H), 7.31-7.23 (m, 2H), 7.20 (dd, J=8.4, 1.9 Hz, 1H). Mass calculated for (C₁₅H₉BrClN₃+H)⁺346.0, found 346.0.

### Synthesis of Compound 110

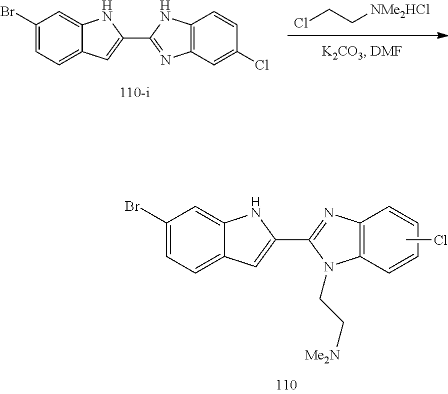

### Compound 110: 2-(2-(6-Bromo-1H-indol-2-yl)-(5 or 6)-chloro-1H-benzo[d]imidazol-1-yl)-N,N-dimethylethan-1-amine

A mixture of 110-i (100 mg, 0.29 mmol), 2-chloro-N,N-dimethylethan-1-amine hydrochloride (42 mg, 0.29 mmol) and K₂CO₃ (80 mg, 0.58 mmol) in DMF (1.5 mL) was stirred at rt for 7 d. The mixture was purified by preparative HPLC (ACN/H₂O with 0.1% formic acid) to give the desired product as 4:1 mixture of regioisomers (9 mg, 7%).

Major isomer: ¹H NMR (400 MHz, DMSO-d₆) δ 12.26 (s, 1H), 7.85 (d, J=2.0 Hz, 1H), 7.70 (d, J=8.6 Hz, 1H), 7.69-7.64 (m, 2H), 7.30 (dd, J=8.6, 2.0 Hz, 1H), 7.23-7.17 (m, 2H), 4.64 (t, J=6.5 Hz, 2H), 2.70 (t, J=6.5 Hz, 2H), 2.20 (s, 6H). Mass calculated for (C₁₉H₁₈BrClN₄+H)⁺419.0, found 419.0.

### Synthesis of Compound 111

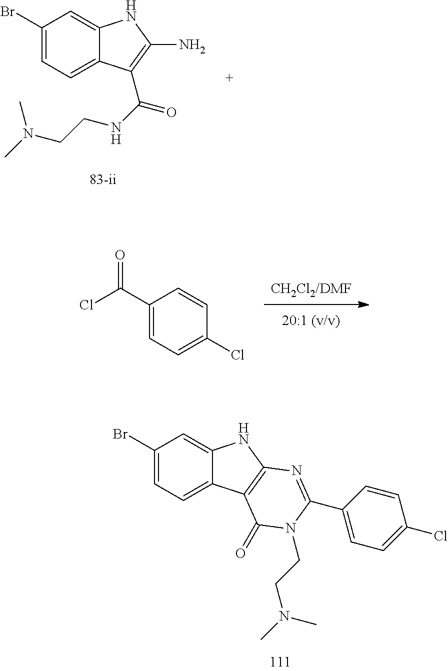 [SMILES: CN(C)CCNC(=O)c1c(N)[nH]c2cc(Br)ccc12 ; O=C(Cl)c1ccc(Cl)cc1 ; CN(C)CCn1c(-c2ccc(Cl)cc2)nc2[nH]c3cc(Br)ccc3c2c1=O]

### Compound 111: 7-bromo-2-(4-chlorophenyl)-3-(2-(dimethylamino)ethyl)-3H-pyrimido[4,5-b]indol-4(9H)-one

A mixture of 2-aminoindole intermediate (83-ii), 4-chlorobenzoyl chloride (1.1 equiv), CH₂Cl₂, and DMF was stirred at ambient temperature for 48 h. The mixture was concentrated in vacuo and purified by column chromatography with 1-5% (5% NH-₄OH/MeOH) in CH₂Cl₂ to afford the desired product as an off-white solid (8 mg, 52%). ¹H NMR (400 MHz, DMSO) δ 12.35 (s, 1H), 7.98 (d, J=8.4 Hz, 1H), 7.72 (d, J=8.5 Hz, 2H), 7.69-7.61 (m, 3H), 7.44 (dd, J=8.3, 1.8 Hz, 1H), 4.08 (t, J=7.0 Hz, 2H), 2.37 (t, J=7.0 Hz, 2H), 1.92 (s, 6H). Mass calculated for (C₂₀H₁₈BrClN₄O+H)⁺445.0, found 445.3.

### Compound 112: 7-bromo-3-(4-chlorophenyl)-2-phenyl-5H-pyrido[3,2-b]indole

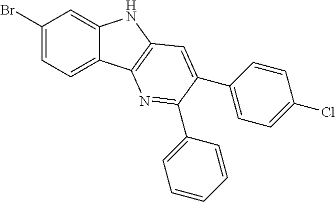 [SMILES: Clc1ccc(-c2cc3[nH]c4cc(Br)ccc4c3nc2-c2ccccc2)cc1]

Prepared according to general method XXV from 92-ii and found as a byproduct (3.1 mg, 0.6%). ¹H NMR (400 MHz, Methanol-d₄) δ 8.41-8.34 (m, 2H), 7.93 (d, J=1.6 Hz, 1H), 7.57 (dd, J=8.6, 1.7 Hz, 1H), 7.51-7.43 (m, 5H), 7.36 (d, J=8.5 Hz, 2H), 7.30 (d, J=8.6 Hz, 2H). Mass calculated for (C₂₃H₁₄BrClN₂+H)⁺435.0, found 435.0.

### Compound 113: 7-bromo-3-(4-chlorophenyl)-2-methyl-5H-pyrido[3,2-b]indole

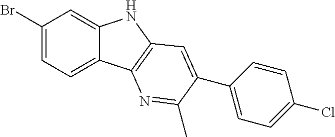 [SMILES: Cc1nc2c(cc1-c1ccc(Cl)cc1)[nH]c1cc(Br)ccc12]

Prepared according to general method XXV from 93-ii and found as a byproduct (3.3 mg, 1.0%). ¹H NMR (400 MHz, Acetone-d₆) δ 10.90 (s, 1H), 8.28 (d, J=8.4 Hz, 1H), 7.88 (d, J=2.1 Hz, 1H), 7.85 (s, 1H), 7.63-7.50 (m, 4H), 7.46 (dd, J=8.4, 1.7 Hz, 1H), 2.66 (s, 3H). Mass calculated for (C₁₈H₁₂BrClN₂+H)⁺373.0, found 373.1.

### Compound 114: 7-bromo-3-(4-chlorophenyl)-2-isopropyl-5H-pyrido[3,2-b]indole

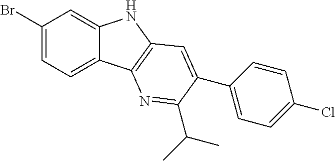 [SMILES: CC(C)c1nc2c(cc1-c1ccc(Cl)cc1)[nH]c1cc(Br)ccc12]

Prepared according to general method XXV from 94-ii and found as a byproduct (1.7 mg, 0.5%). ¹H NMR (400 MHz, Acetone-d₆) δ 8.27 (d, J=8.3 Hz, 1H), 7.82 (d, J=1.7 Hz, 1H), 7.74 (s, 1H), 7.56 (d, J=8.4 Hz, 2H), 7.48 (d, J=8.5 Hz, 2H), 7.45 (dd, J=8.3, 1.7 Hz, 1H), 3.31 (hept, J=7.0 Hz, 1H), 1.32 (d, J=6.7 Hz, 6H). Mass calculated for (C₁₀H₁₆BrClN₂+H)⁺401.0, found 401.1.

### Synthesis of Compound 115

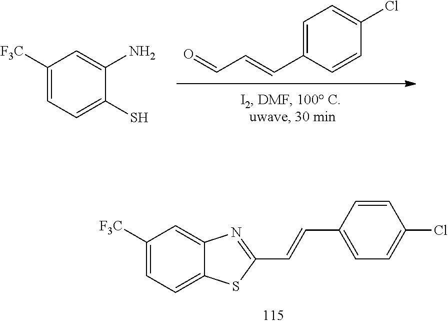 [SMILES: Nc1cc(C(F)(F)F)ccc1S ; O=C/C=C/c1ccc(Cl)cc1 ; FC(F)(F)c1ccc2sc(/C=C/c3ccc(Cl)cc3)nc2c1]

### Compound 115: (E)-2-(4-chlorostyryl)-5-(trifluoromethyl)benzo[d]thiazole

2-Amino-4-trifluoromethylbenzenethiol (1.0 eq), 4-chlorocinnamaldehyde (1.0 eq) and iodine (0.5 eq) in DMF (0.2 M) and heated to 100° C. in a microwave reactor. Upon cooling, water was added, and the precipitate was filtered off or extracted with EtOAc. When extracted, the precipitate was washed twice with NaOH, once with brine, dried over sodium sulfate, and concentrated under reduced pressure. The residue was purified by silica gel chromatography in EtOAc/hexanes to give 115 (22.6 mg, 17%). ¹H NMR (400 MHz, DMSO-d₆) δ 8.39 (d, J=8.4 Hz, 1H), 8.33 (s, 1H), 7.86 (d, J=8.6 Hz, 2H), 7.81-7.75 (m, 2H), 7.72 (d, J=16.3 Hz, 1H), 7.53 (d, J=8.5 Hz, 2H). Mass calculated for (C₁₆H₉ClF₃NS+H)⁺340.0, found 340.4.

### General Method XXXI

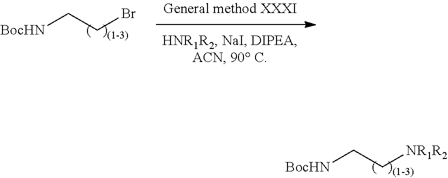

A mixture of the corresponding bromide (1.0 mmol), NaI (0.2 mmol), DIPEA (2.0 mmol) and the corresponding amine (1.2 mmol) in ACN (4.0 mL) was heated in a sealed tube at 90° C. for 18 h and then concentrated under reduced pressure. The residue was purified by silica gel chromatography, eluting with MeOH/DCM gradient, to provide the desired amine intermediates.

### Intermediate 116-i: Tert-Butyl (S)-(4-(2-carbamoylpyrrolidin-1-yl)butyl)carbamate

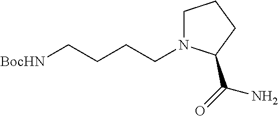 [SMILES: CC(C)(C)OC(=O)NCCCCN1CCC[C@H]1C(N)=O]

Prepared according to general method XXXI from tert-butyl (4-bromobutyl)carbamate and (S)-pyrrolidine-2-carboxamide (135 mg, 48%). ¹H NMR (400 MHz, DMSO-d₆) δ 7.07 (s, 1H), 7.03 (s, 1H), 6.79 (t, J=5.1 Hz, 1H), 3.08 (bs, 1H), 2.91 (q, J=6.3 Hz, 2H), 2.81-2.71 (m, 1H), 2.36-1.92 (m, 3H), 1.80-1.61 (m, 3H), 1.48-1.39 (m, 5H), 1.38 (s, 9H).

### Intermediate 117-i: Tert-Butyl (4-(4-methyl-1,4-diazepan-1-yl)butyl)carbamate

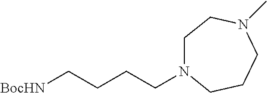 [SMILES: CN1CCCN(CCCCNC(=O)OC(C)(C)C)CC1]

Prepared according to general method XXXI from tert-butyl (4-bromobutyl)carbamate and 1-methyl-1,4-diazepane (210 mg, 74%). ¹H NMR (400 MHz, DMSO-d₆) δ 6.82 (t, J=5.7 Hz, 1H), 3.14-2.76 (m, 10H), 2.72-2.65 (m, 2H), 2.64 (s, 3H), 1.91 (bs, 2H), 1.52-1.40 (m, 3H), 1.38 (s, 9H), 1.34-1.19 (m, 1H).

### General Method XXXII

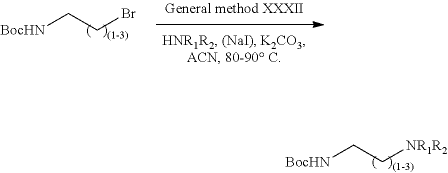

A mixture of the corresponding bromide (1.0 mmol), K₂CO₃ (2.0-3.0 mmol), the corresponding amine (1.2 mmol) and optionally NaI (0.2 mmol) in ACN (4.0 mL) was heated in a sealed tube at 80-90° C. for 18 h and then concentrated under reduced pressure. The residue was purified by silica gel chromatography, eluting with either MeOH/DCM or ([5% NH₄OH/MeOH]/DCM) gradient, to provide the desired amine intermediates.

### Intermediate 118-i: Tert-Butyl (3-(4-methyl-1,4-diazepan-1-yl)propyl)carbamate

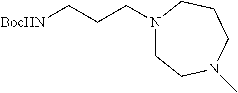 [SMILES: CN1CCCN(CCCNC(=O)OC(C)(C)C)CC1]

Prepared according to general method XXXII from tert-butyl (3-bromopropyl)carbamate and 1-methyl-1,4-diazepane with NaI and K₂CO₃ (2.0 mmol) (104 mg, 37%). ¹H NMR (400 MHz, DMSO-d₆) δ 6.88-6.69 (m, 1H), 3.14-2.85 (m, 8H), 2.79 (t, J=5.9 Hz, 2H), 2.66 (s, 3H), 2.59 (t, J=7.3 Hz, 2H), 1.97-1.87 (m, 2H), 1.56 (p, J=7.3 Hz, 2H), 1.36 (s, 9H).

### Intermediate 119-i: Tert-Butyl (2-(4-methyl-1,4-diazepan-1-yl)ethyl)carbamate

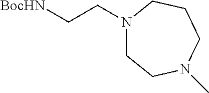 [SMILES: CN1CCCN(CCNC(=O)OC(C)(C)C)CC1]

Prepared according to general method XXXII from tert-butyl (2-bromoethyl)carbamate and 1-methyl-1,4-diazepane with NaI and K₂CO₃ (2.0 mmol) (185 mg, 64%). ¹H NMR (400 MHz, DMSO-d₆) δ 6.67 (t, J=5.9 Hz, 1H), 3.01 (q, J=6.4 Hz, 2H), 2.91-2.76 (m, 4H), 2.75-2.70 (m, 2H), 2.68 (t, J=6.1 Hz, 2H), 2.48 (s, 3H), 1.83-1.71 (m, 2H), 1.44-1.39 (m, 2H), 1.38 (s, 9H).

### Intermediate 120-i: Tert-Butyl (S)-(2-(2-carbamoylpyrrolidin-1-yl)ethyl)carbamate

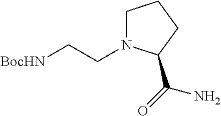 [SMILES: CC(C)(C)OC(=O)NCCN1CCC[C@H]1C(N)=O]

Prepared according to general method XXXII from tert-butyl (2-bromoethyl)carbamate and (S)-pyrrolidine-2-carboxamide with NaI and K₂CO₃ (2.0 mmol) (120 mg, 42%). ¹H NMR (400 MHz, DMSO-d₆) δ 7.25 (s, 1H), 7.06 (s, 1H), 6.85 (t, J=4.9 Hz, 1H), 3.19-2.89 (m, 3H), 2.85-2.76 (m, 1H), 2.60 (dt, J=12.0, 7.5 Hz, 1H), 2.36 (dt, J=11.6, 5.6 Hz, 1H), 2.27-2.15 (m, 1H), 2.07-1.93 (m, 1H), 1.76-1.56 (m, 3H), 1.37 (s, 9H).

### Intermediate 121-i: Tert-Butyl (2-(4-(2-methoxyacetyl)-1,4-diazepan-1-yl)ethyl)carbamate

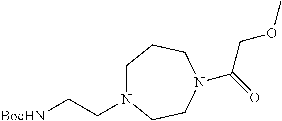 [SMILES: COCC(=O)N1CCCN(CCNC(=O)OC(C)(C)C)CC1]

Prepared according to general method XXXII from tert-butyl (2-bromoethyl)carbamate and 1-(1,4-diazepan-1-yl)-2-methoxyethan-1-one hydrochloride with K₂CO₃ (2.0 mmol) (140 mg, 40%). ¹H NMR (400 MHz, DMSO-d₆) δ 6.71-6.54 (m, 1H), 4.06 (d, J=2.9 Hz, 2H), 3.50-3.36 (m, 4H), 3.28 (s, 3H), 3.00 (q, J=7.2 Hz, 2H), 2.74-2.65 (m, 1H), 2.64-2.54 (m, 3H), 2.49-2.41 (m, 2H), 1.83-1.61 (m, 2H), 1.38 (s, 9H).

### Intermediate 122-i: Tert-Butyl (2-((2S,5R)-2,4,5-trimethylpiperazin-1-yl)ethyl)carbamate

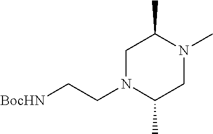 [SMILES: C[C@@H]1CN(CCNC(=O)OC(C)(C)C)[C@@H](C)CN1C]

Prepared according to general method XXXII from tert-butyl (2-bromoethyl)carbamate and (2R,5S)-1,2,5-trimethylpiperazine oxalate with K₂CO₃ (3.0 mmol) (31 mg, 31%). ¹H NMR (400 MHz, DMSO-d₆) δ 6.65 (s, 1H), 3.10-2.86 (m, 2H), 2.80-2.56 (m, 3H), 2.41-1.64 (m, 8H), 1.38 (s, 9H), 0.95 (s, 3H), 0.94 (s, 3H).

### Intermediate 123-i: Tert-butyl (2-(2-(2-methoxyethyl)pyrrolidin-1-yl)ethyl)carbamate

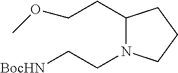 [SMILES: COCCC1CCCN1CCNC(=O)OC(C)(C)C]

Prepared according to general method XXXII from tert-butyl (2-bromoethyl)carbamate and 2-(2-methoxyethyl)pyrrolidine with K₂CO₃ (2.0 mmol) (178 mg, 86%). ¹H NMR (400 MHz, DMSO-d₆) δ 6.66 (s, 1H), 3.41-3.34 (m, 2H), 3.22 (s, 3H), 3.13-2.70 (m, 4H), 2.40-2.24 (m, 1H), 2.16-1.97 (m, 2H), 1.91-1.57 (m, 4H), 1.47-1.31 (m, 2H).

### Intermediate 124-i: Tert-Butyl (2-((1-(2-methoxyethyl)piperidin-4-yl)amino)ethyl)carbamate

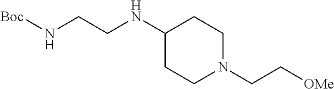 [SMILES: COCCN1CCC(NCCNC(=O)OC(C)(C)C)CC1]

Prepared according to general method XXXII from tert-butyl (2-bromoethyl)carbamate, 1-(2-methoxyethyl)piperidin-4-amine and K₂CO₃ (177 mg, 60%). ¹H NMR (400 MHz, CDCl₃) δ 5.02 (s, 1H), 3.55 (t, J=5.6 Hz, 2H), 3.51 (s, 3H), 3.37 (s, 3H), 3.24 (d, J=6.1 Hz, 2H), 2.98 (d, J=11.6 Hz, 2H), 2.79 (t, J=5.9 Hz, 2H), 2.62 (t, J=5.7 Hz, 2H), 2.14 (t, J=11.5 Hz, 2H), 1.93 (d, J=12.7 Hz, 2H), 1.47 (s, 9H). Mass calculated for (C₁₅H₃₁N₃O₃+H)⁺ 302.2, found 302.2.

### Intermediate 125-i: Tert-Butyl (2-((tert-butoxycarbonyl)amino)ethyl)-L-prolinate

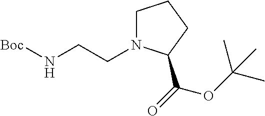 [SMILES: CC(C)(C)OC(=O)NCCN1CCC[C@H]1C(=O)OC(C)(C)C]

Prepared according to general method XXXII from tert-butyl (2-bromoethyl)carbamate, tert-butyl L-prolinate and K₂CO₃ (112 mg, 36%). ¹H NMR (400 MHz, CDCl₃) δ 5.35 (s, 1H), 3.29-3.05 (m, 4H), 2.78 (dt, J=12.6, 7.0 Hz, 1H), 2.64 (d, J=12.3 Hz, 1H), 2.39 (d, J=8.3 Hz, 1H), 2.18-2.03 (m, 1H), 1.92 (dt, J=10.7, 5.2 Hz, 2H), 1.82 (t, J=8.2 Hz, 1H), 1.49 (s, 9H), 1.47 (s, 9H). Mass calculated for (C₁₆H₃₀N₂O₄+H)⁺315.2, found 315.2.

### Synthesis of Intermediate 126-i

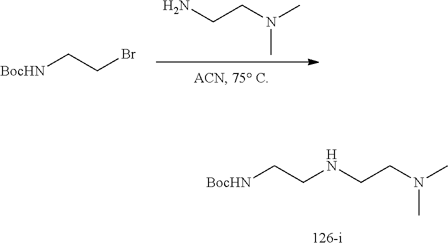

### Intermediate 126-i: Tert-Butyl (2-((2-(dimethylamino)ethyl)amino)ethyl)carbamate

A mixture of tert-butyl (2-bromoethyl)carbamate (250 mg, 1.1 mmol) and N¹,N¹-dimethylethane-1,2-diamine (1.5 mL, 13.7 mmol) in ACN (3.0 mL) was heated at 75° C. for 18 h and then concentrated. The residue was purified by silica gel chromatography, eluting with MeOH (5% aqueous NH₄OH)/DCM gradient, to provide the desired amine intermediate 126-i (180 mg, 70%). ¹H NMR (400 MHz, DMSO-d₆) δ 6.79 (t, J=5.5 Hz, 1H), 3.04 (q, J=6.3 Hz, 2H), 2.70-2.57 (m, 4H), 2.35 (t, J=6.3 Hz, 2H), 2.17 (s, 6H), 1.39 (s, 9H).

### Intermediate 127-i: Tert-butyl 2-(3-oxopiperazin-1-yl)ethylcarbamate

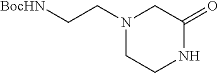 [SMILES: CC(C)(C)OC(=O)NCCN1CCNC(=O)C1]

Prepared according to general method XXXII from tert-butyl N-(2-bromoethyl)carbamate and piperazin-2-one to yield 55.4 mg (51%). ¹H NMR (400 MHz, Chloroform-d) δ 4.95 (d, J=441.9 Hz, 1H), 3.51 (ddd, J=2376.0, 48.0, 4.3 Hz, 1H), 3.40 (ddd, J=6.3, 4.9, 2.2 Hz, 2H), 3.28 (q, J=5.8 Hz, 2H), 3.18 (s, 2H), 2.71 (d, J=5.6 Hz, 2H), 2.58 (t, J=6.0 Hz, 2H), 1.47 (s, 9H).

### Intermediate 128-i: Tert-butyl 2-(4-carbamoyl-1,4-diazepan-1-yl)ethylcarbamate

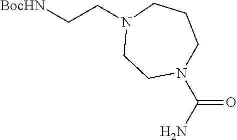 [SMILES: CC(C)(C)OC(=O)NCCN1CCCN(C(N)=O)CC1]

Prepared according to general method XXXII from tert-butyl N-(2-bromoethyl)carbamate and 1,4-diazepane-1-carboxamide to yield 63.2 mg (49%). ¹H NMR (400 MHz, Chloroform-d) δ 5.01 (s, 1H), 4.52 (s, 2H), 3.57 (s, 2H), 3.49 (s, 2H), 3.23 (s, 2H), 2.85-2.67 (m, 4H), 2.63 (t, J=5.9 Hz, 2H), 1.92 (s, 2H), 1.47 (s, 9H).

### Intermediate 129-i: Tert-butyl 2-(4-(2-methoxyethyl)piperazin-1-yl)ethylcarbamate

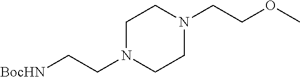 [SMILES: COCCN1CCN(CCNC(=O)OC(C)(C)C)CC1]

Prepared according to general method XXXII from tert-butyl N-(2-bromoethyl)carbamate and 1-(2-methoxyethyl)piperazine to yield 54.7 mg (43%). ¹H NMR (400 MHz, Chloroform-d) δ 5.00 (s, 1H), 3.53 (t, J=5.6 Hz, 2H), 3.37 (s, 3H), 3.31-3.14 (m, 2H), 2.60 (t, J=5.7 Hz, 2H), 2.58-2.51 (m, 8H), 2.48 (t, J=6.1 Hz, 2H), 1.47 (s, 9H).

### Intermediate 130-i: Tert-butyl 2-(4-(2-methoxyethyl)-1,4-diazepan-1-yl)ethylcarbamate

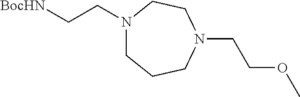 [SMILES: COCCN1CCCN(CCNC(=O)OC(C)(C)C)CC1]

Prepared according to general method XXXII from tert-butyl N-(2-bromoethyl)carbamate and 1-(2-methoxyethyl)-1,4-diazepine to yield 90 mg (67%). ¹H NMR (400 MHz, Chloroform-d) δ 5.06 (s, 1H), 3.55 (t, J=5.7 Hz, 2H), 3.37 (s, 3H), 3.29-3.12 (m, 2H), 2.81 (ddd, J=25.1, 12.8, 5.7 Hz, 10H), 2.63 (t, J=6.1 Hz, 2H), 1.87 (p, J=6.0 Hz, 2H), 1.47 (s, 9H).

### Intermediate 131-i: Tert-butyl 2-(1-carbamoylpiperidin-4-ylamino)ethylcarbamate

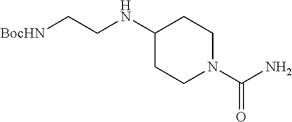 [SMILES: CC(C)(C)OC(=O)NCCNC1CCN(C(N)=O)CC1]

Prepared according to general method XXXII from tert-butyl N-(2-bromoethyl)carbamate and 4-aminopiperidine-1-carboxamide to yield 110 mg (43%). ¹H NMR (400 MHz, Methanol-d₄) δ 4.18-3.98 (m, 2H), 3.94 (d, J=13.6 Hz, 1H), 3.11-2.72 (m, 4H), 2.03-1.81 (m, 4H), 1.56-1.40 (m, 9H), 1.44-1.21 (m, 2H).

### Intermediate 132-i: Tert-butyl 2-(4-carbamoylpiperazin-1-yl)ethylcarbamate

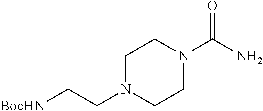 [SMILES: CC(C)(C)OC(=O)NCCN1CCN(C(N)=O)CC1]

Prepared according to general method XXXII from tert-butyl N-(2-bromoethyl)carbamate and 1-piperazinecarboxamide to yield 80 mg (33%). ¹H NMR (400 MHz, Chloroform-d) δ 4.95 (s, 1H), 4.48 (s, 2H), 3.43 (t, J=5.7 Hz, 4H), 3.27 (q, J=6.2 Hz, 2H), 2.59-2.39 (m, 6H), 1.48 (s, 9H).

### General Method XXXIII

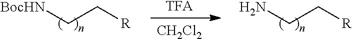

To a stirred solution of the appropriate Boc-aminoalkylamine from general method XXXI and XXXII in DCM (˜0.1 M) was added trifluoroacetic acid (25-50% TFA/DCM). The deprotection was monitored by HPLC. When complete, the solution was concentrated in vacuo and co-evaporated with DCM and optionally toluene to remove excess TFA. The crude amine was used without purification.

### General Method XXXIV

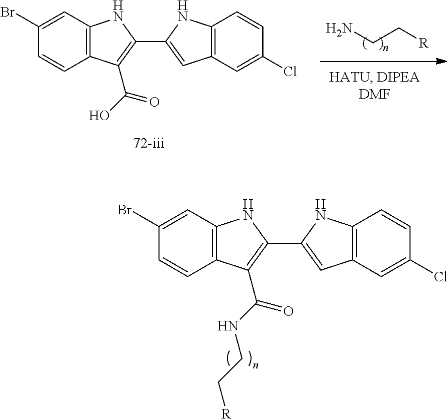

To a stirred solution of intermediate 72-iii (1.0 eq) in DMF (0.5 M) was added HATU (1.2 eq), then DIPEA (5-6 eq). After 3-5 min, a solution of the appropriate amine from general method XXXIII in DMF (1.1 eq, 0.5 M) was added. The reactions were allowed to stir overnight, wherein they were diluted with EtOAc, washed with NaHCO₃₍ₛₐₜ₎ and/or water, brine, dried over sodium sulfate, filtered and concentrated under reduced pressure. In some cases, trituration in DCM yielded pure products as solids. In others, the residue was purified by silica gel chromatography, eluting with ([5% NH₄OH/MeOH]/DCM) gradient, to yield pure product.

### Compound 116: (S)-6-Bromo-N-(4-(2-carbamoylpyrrolidin-1-yl)butyl)-5′-chloro-1H,1′H-[2,2′-biindole]-3-carboxamide

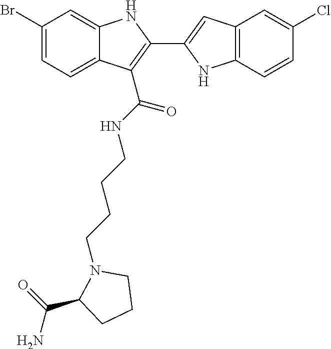 [SMILES: NC(=O)[C@@H]1CCCN1CCCCNC(=O)c1c(-c2cc3cc(Cl)ccc3[nH]2)[nH]c2cc(Br)ccc12]

Prepared according to general methods XXXIII and XXXIV from intermediate 116-i and intermediate 72-iii (86 mg, 60%). ¹H NMR (400 MHz, DMSO-d₆) δ 12.53 (s, 1H), 12.28 (s, 1H), 8.34 (t, J=5.6 Hz, 1H), 7.76-7.69 (m, 2H), 7.66-7.57 (m, 2H), 7.34 (dd, J=8.6, 1.8 Hz, 1H), 7.19-7.04 (m, 4H), 3.40 (q, J=6.6 Hz, 2H), 3.08 (s, 1H), 2.79 (d, J=9.2 Hz, 1H), 2.61 (dt, J=11.5, 7.7 Hz, 1H), 2.37 (dt, J=12.2, 6.5 Hz, 1H), 2.19 (q, J=8.1 Hz, 1H), 2.07-1.92 (m, 1H), 1.77-1.49 (m, 7H). Mass calculated for (C₂₆H₂₇BrClN₅O₂+H)⁺556.1, found 556.0.

### Compound 117: 6-Bromo-5′-chloro-N-(4-(4-methyl-1,4-diazepan-1-yl)butyl)-1H,1′H-[2,2′-biindole]-3-carboxamide

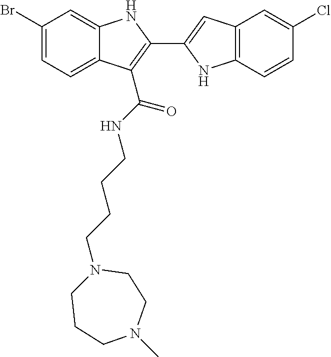 [SMILES: CN1CCCN(CCCCNC(=O)c2c(-c3cc4cc(Cl)ccc4[nH]3)[nH]c3cc(Br)ccc23)CC1]

Prepared according to general methods XXXIII and XXXIV from intermediate 117-i and intermediate 72-iii (26 mg, 18%). ¹H NMR (400 MHz, DMSO-d₆) δ 12.56 (s, 1H), 12.50 (s, 1H), 8.36 (s, 1H), 7.75-7.69 (m, 2H), 7.66 (d, J=1.8 Hz, 1H), 7.60 (d, J=8.7 Hz, 1H), 7.33 (dd, J=8.6, 1.8 Hz, 1H), 7.23-7.12 (m, 2H), 3.50-3.39 (m, 5H), 3.00-2.85 (m, 6H), 2.82 (t, J=5.8 Hz, 2H), 2.71-2.63 (m, 2H), 1.91-1.82 (m, 2H), 1.71-1.54 (m, 4H). Mass calculated for (C₂₇H₃₁BrClN₅O+H)⁺556.1, found 556.0.

### Compound 118: 6-Bromo-5′-chloro-N-(3-(4-methyl-1,4-diazepan-1-yl)propyl)-1H,1′H-[2,2′-biindole]-3-carboxamide

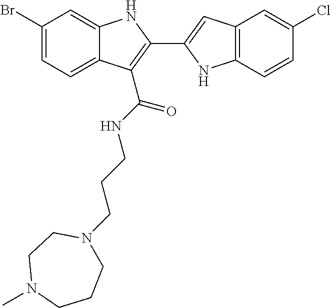 [SMILES: CN1CCCN(CCCNC(=O)c2c(-c3cc4cc(Cl)ccc4[nH]3)[nH]c3cc(Br)ccc23)CC1]

Prepared according to general methods XXXII and XXXIV from intermediate 118-i and intermediate 72-iii (45 mg, 32%). ¹H NMR (400 MHz, DMSO-d₆) δ 12.56 (bs, 1H), 12.29 (bs, 1H), 8.36 (bs, 1H), 7.74 (d, J=8.7 Hz, 1H), 7.72 (d, J=2.1 Hz, 1H), 7.63 (d, J=1.8 Hz, 1H), 7.61 (d, J=8.7 Hz, 1H), 7.33 (dd, J=8.6, 1.8 Hz, 1H), 7.16 (dd, J=8.7, 2.1 Hz, 1H), 7.14 (s, 1H), 3.44 (t, J=6.9 Hz, 2H), 2.66-2.58 (m, 4H), 2.56-2.52 (m, 2H), 2.49-2.41 (m, 4H), 2.20 (s, 3H), 1.74 (p, J=6.9 Hz, 2H), 1.66 (p, J=6.0 Hz, 2H). Mass calculated for (C₂₆H₂₉BrClN₅O+H)⁺542.1, found 542.0.

### Compound 119: 6-Bromo-5′-chloro-N-(2-(4-methyl-1,4-diazepan-1-yl)ethyl)-1H,1′H-[2,2′-biindole]-3-carboxamide

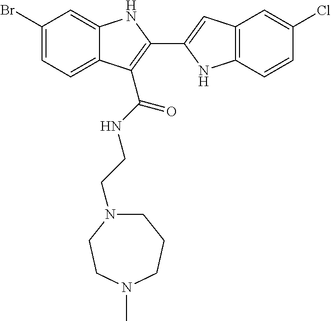 [SMILES: CN1CCCN(CCNC(=O)c2c(-c3cc4cc(Cl)ccc4[nH]3)[nH]c3cc(Br)ccc23)CC1]

Prepared according to general methods XXXIII and XXXIV from intermediate 119-i and intermediate 72-iii (52 mg, 38%). ¹H NMR (400 MHz, DMSO-d₆) δ 12.57 (s, 1H), 12.37 (s, 1H), 8.11 (t, J=5.5 Hz, 1H), 7.88 (d, J=8.7 Hz, 1H), 7.73 (d, J=2.0 Hz, 1H), 7.65 (d, J=1.8 Hz, 1H), 7.61 (d, J=8.7 Hz, 1H), 7.32 (dd, J=8.6, 1.8 Hz, 1H), 7.20-7.10 (m, 2H), 3.49 (q, J=6.0 Hz, 2H), 2.81-2.68 (m, 10H), 2.37 (s, 3H), 1.84-1.74 (m, 2H). Mass calculated for (C₂₅H₂₇BrClN₅O+H)⁺528.1, found 527.9.

### Compound 120: (S)-6-Bromo-N-(2-(2-carbamoylpyrrolidin-1-yl)ethyl)-5′-chloro-1H,1′H-[2,2′-biindole]-3-carboxamide

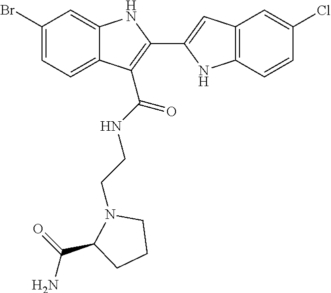 [SMILES: NC(=O)[C@@H]1CCCN1CCNC(=O)c1c(-c2cc3cc(Cl)ccc3[nH]2)[nH]c2cc(Br)ccc12]

Prepared according to general methods XXXII and XXXIV from intermediate 120-i and intermediate 72-iii (71 mg, 52%). ¹H NMR (400 MHz, DMSO-d₆) δ 12.56 (s, 1H), 12.34 (s, 1H), 8.35 (t, J=5.5 Hz, 1H), 7.81 (d, J=8.7 Hz, 1H), 7.72 (d, J=2.0 Hz, 1H), 7.63 (d, J=1.9 Hz, 1H), 7.61 (d, J=8.9 Hz, 1H), 7.33 (dd, J=8.6, 1.8 Hz, 1H), 7.29 (d, J=3.5 Hz, 1H), 7.19-7.13 (m, 2H), 7.08 (d, J=3.5 Hz, 1H), 3.63-3.44 (m, 2H), 3.28-3.16 (m, 1H), 3.00-2.82 (m, 2H), 2.68-2.59 (m, 1H), 2.36 (q, J=8.2 Hz, 1H), 2.15-1.97 (m, 1H), 1.82-1.61 (m, 3H). Mass calculated for (C₂₄H₂₃BrClN₅O₂+H)⁺528.1, found 527.9.

### Compound 121: 6-Bromo-5′-chloro-N-(2-(4-(2-methoxyacetyl)-1,4-diazepan-1-yl)ethyl)-1H,1′H-[2,2′-biindole]-3-carboxamide

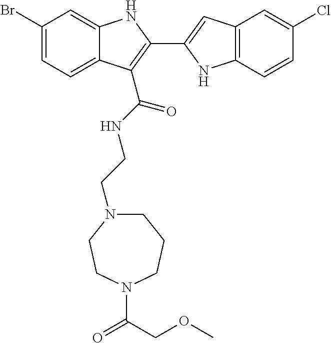 [SMILES: COCC(=O)N1CCCN(CCNC(=O)c2c(-c3cc4cc(Cl)ccc4[nH]3)[nH]c3cc(Br)ccc23)CC1]

Prepared according to general methods XXXIII and XXXIV from intermediate 121-i and intermediate 72-iii (63 mg, 42%). Present as mixture of rotomers. H NMR (400 MHz, DMSO-d₆) δ 12.57 (s, 1H), 12.30 (s, 1H), 8.19-8.07 (m, 1H), 7.86 (d, J=8.6 Hz, 1H), 7.73 (d, J=2.0 Hz, 1H), 7.66-7.57 (m, 2H), 7.37-7.29 (m, 1H), 7.20-7.12 (m, 2H), 4.07 (s, 1H), 4.04 (s, 1H), 3.57-3.39 (m, 6H), 3.28 (s, 1.5H), 3.24 (s, 1.5H), 2.83-2.61 (m, 6H), 1.77 (m, 2H). Mass calculated for (C₂₇H₂₉BrClN₅O₃+H)⁺586.1, found 586.0.

### Compound 122: 6-Bromo-5′-chloro-N-(2-((2S,5R)-2,4,5-trimethylpiperazin-1-yl)ethyl)-1H,1′H-[2,2′-biindole]-3-carboxamide

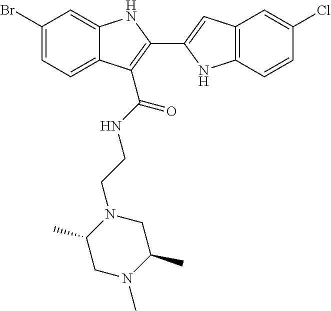 [SMILES: C[C@@H]1CN(CCNC(=O)c2c(-c3cc4cc(Cl)ccc4[nH]3)[nH]c3cc(Br)ccc23)[C@@H](C)CN1C]

Prepared according to general methods XXXIII and XXXIV from intermediate 122-i and intermediate 72-iii (30 mg, 54%). ²H NMR (400 MHz, Methanol-d₄) δ 7.87 (d, J=8.6 Hz, 1H), 7.62 (d, J=1.7 Hz, 1H), 7.59 (d, J=1.9 Hz, 1H), 7.43 (d, J=8.7 Hz, 1H), 7.29 (dd, J=8.6, 1.7 Hz, 1H), 7.14 (dd, J=8.7, 2.0 Hz, 1H), 7.00 (s, 1H), 3.75-3.52 (m, 2H), 3.22-3.09 (m, 1H), 2.99 (d, J=8.9 Hz, 1H), 2.72 (dd, J=11.7, 2.8 Hz, 1H), 2.55-2.34 (m, 2H), 2.24 (s, 3H), 2.18-2.03 (m, 2H), 1.95 (t, J=11.1 Hz, 1H), 1.07 (s, 3H), 1.06 (s, 3H). Mass calculated for (C₂₆H₂₉BrClN₅O+H)⁺542.1, found 542.0.

### Compound 123: 6-Bromo-5′-chloro-N-(2-(2-(2-methoxyethyl)pyrrolidin-1-yl)ethyl)-1H,1′H-[2,2′-biindole]-3-carboxamide

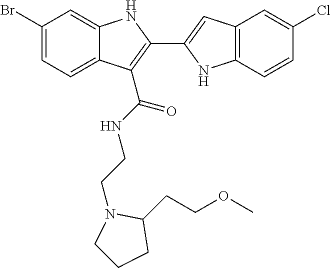 [SMILES: COCCC1CCCN1CCNC(=O)c1c(-c2cc3cc(Cl)ccc3[nH]2)[nH]c2cc(Br)ccc12]

Prepared according to general methods XXXIII and XXXIV from intermediate 123-i and intermediate 72-iii (93 mg, 67%). ¹H NMR (400 MHz, DMSO-d₆) δ 12.55 (s, 1H), 12.29 (s, 1H), 8.11 (bs, 1H), 7.87 (d, J=8.7 Hz, 1H), 7.73 (d, J=2.1 Hz, 1H), 7.63 (d, J=1.8 Hz, 1H), 7.60 (d, J=8.7 Hz, 1H), 7.31 (dd, J=8.6, 1.8 Hz, 1H), 7.17 (dd, J=8.7, 2.1 Hz, 1H), 7.14 (s, 1H), 3.67-3.38 (m, 2H), 3.32-3.24 (m, 2H), 3.24-3.16 (m, 1H), 3.14 (s, 3H), 3.11-3.02 (m, 1H), 2.44-2.23 (m, 2H), 2.14 (q, J=7.7 Hz, 1H), 1.93-1.59 (m, 4H), 1.49-1.31 (m, 2H). Mass calculated for (C₂₆H₂₈BrClN₄O₂+H)⁺ 543.1, found 543.0.

### Compound 124: 6-Bromo-5′-chloro-N-(2-((1-(2-methoxyethyl)piperidin-4-yl)amino)ethyl)-1H,1′H-[2,2′-biindole]-3-carboxamide

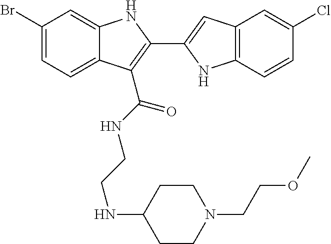 [SMILES: COCCN1CCC(NCCNC(=O)c2c(-c3cc4cc(Cl)ccc4[nH]3)[nH]c3cc(Br)ccc23)CC1]

Prepared according to general methods XXXII and XXXIV from intermediate 124-i and intermediate 72-iii (101 mg, 69%). ¹H NMR (400 MHz, DMSO-d₆) δ 12.33 (s, 1H), 8.24 (t, J=5.6 Hz, 1H), 7.83 (d, J=8.6 Hz, 1H), 7.73 (d, J=2.1 Hz, 1H), 7.64 (d, J=1.8 Hz, 1H), 7.59 (d, J=8.7 Hz, 1H), 7.32 (dd, J=8.6, 1.8 Hz, 1H), 7.22-7.14 (m, 2H), 3.54 (q, J=6.2 Hz, 2H), 3.41 (t, J=5.8 Hz, 2H), 3.23 (s, 3H), 2.92 (t, J=6.2 Hz, 2H), 2.84 (d, J=11.6 Hz, 2H), 2.67-2.56 (m, 1H), 2.46 (t, J=5.8 Hz, 2H), 2.00 (t, J=11.4 Hz, 2H), 1.82 (bs, J=9.8 Hz, 2H), 1.41-1.26 (m, 2H). Mass calculated for (C₂₇H₃₁BrClN₅O₂+H)⁺572.1, found 572.1.

### Compound 125: (2-(6-Bromo-5′-chloro-1H,1′H-[2,2′-biindole]-3-carboxamido)ethyl)-L-proline

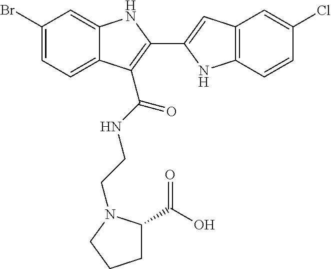 [SMILES: O=C(NCCN1CCC[C@H]1C(=O)O)c1c(-c2cc3cc(Cl)ccc3[nH]2)[nH]c2cc(Br)ccc12]

Prepared according to general methods XXXIII and XXXIV from intermediate 125-i and intermediate 72-iii (45 mg, 33%). ¹H NMR (400 MHz, DMSO-d₆) δ 12.60 (s, 1H), 12.41 (s, 1H), 8.36 (bs, 1H), 7.87 (d, J=8.6 Hz, 1H), 7.74-7.67 (m, 2H), 7.63 (d, J=1.8 Hz, 1H), 7.30 (dd, J=8.6, 1.9 Hz, 1H), 7.14 (dd, J=8.7, 2.0 Hz, 1H), 7.10 (s, 1H), 3.66-3.52 (m, 4H), 3.31-3.21 (m, 1H), 3.15-3.04 (m, 1H), 2.84 (td, J=9.6, 7.0 Hz, 1H), 2.25-2.10 (m, 1H), 2.00-1.65 (m, 3H). Mass calculated for (C₂₄H₂₂BrClN₄O₃+H)⁺529.1, found 529.0.

### Compound 126: 6-Bromo-5′-chloro-N-(2-((2-(dimethylamino)ethyl)amino)ethyl)-1H,1′H-[2,2′-biindole]-3-carboxamide

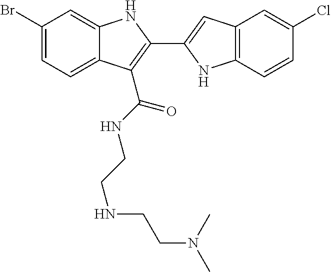 [SMILES: CN(C)CCNCCNC(=O)c1c(-c2cc3cc(Cl)ccc3[nH]2)[nH]c2cc(Br)ccc12]

Prepared according to general methods XXXII and XXXIV from intermediate 126-i and intermediate 72-iii (49 mg, 38%). ¹H NMR (400 MHz, DMSO-d₆) δ 12.48 (s, 2H), 8.41 (t, J=5.6 Hz, 1H), 7.87 (d, J=8.6 Hz, 1H), 7.74 (d, J=2.0 Hz, 1H), 7.66 (d, J=1.8 Hz, 1H), 7.59 (d, J=8.7 Hz, 1H), 7.34 (dd, J=8.6, 1.8 Hz, 1H), 7.23-7.14 (m, 2H), 3.73 (q, J=6.1 Hz, 2H), 3.42-3.31 (m, 4H), 3.25 (t, J=6.2 Hz, 2H), 2.71 (bs, 6H). Mass calculated for (C₂₃H₂₅BrClN₅O+H)⁺502.1, found 502.1.

### Compound 127: 6-bromo-5′-chloro-N-(2-(3-oxopiperazin-1-yl)ethyl)-1H,1′H-2,2′-biindole-3-carboxamide

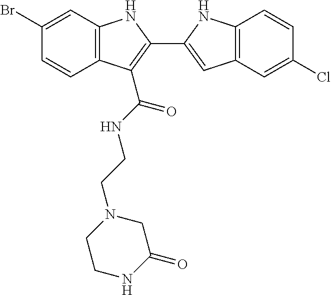 [SMILES: O=C1CN(CCNC(=O)c2c(-c3cc4cc(Cl)ccc4[nH]3)[nH]c3cc(Br)ccc23)CCN1]

Prepared according to general methods XXXIII and XXXIV from intermediate 127-i and intermediate 72-iii to yield 23.7 mg (21%) as a tan powder. ¹H NMR (400 MHz, Methanol-d₄) δ 7.86 (d, J=8.6 Hz, 1H), 7.65 (d, J=1.7 Hz, 1H), 7.61 (d, J=2.0 Hz, 1H), 7.46 (d, J=8.8 Hz, 1H), 7.32 (dd, J=8.6, 1.8 Hz, 1H), 7.17 (dd, J=8.7, 2.0 Hz, 1H), 7.04 (s, 1H), 3.78 (t, J=6.0 Hz, 2H), 3.51 (s, 2H), 3.42 (t, J=5.5 Hz, 2H), 3.21-2.91 (m, 4H). Mass calculated for (C₂₃H₂₁BrClN₅O₂+H)⁺516.1, found 516.2.

### Compound 128: 6-bromo-N-(2-(4-carbamoyl-1,4-diazepan-1-yl)ethyl)-5′-chloro-1H,1′H-2,2′-biindole-3-carboxamide

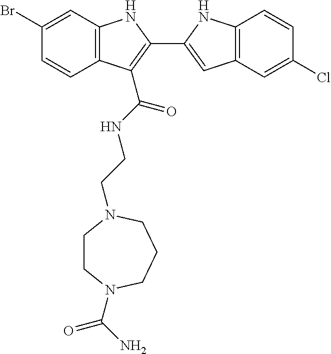 [SMILES: NC(=O)N1CCCN(CCNC(=O)c2c(-c3cc4cc(Cl)ccc4[nH]3)[nH]c3cc(Br)ccc23)CC1]

Prepared according to general methods XXXIII and XXXIV from intermediate 128-i and intermediate 72-iii to yield 50 mg (45%). ¹H NMR (400 MHz, Methanol-d₄) δ 7.85 (d, J=8.6 Hz, 1H), 7.64 (d, J=1.7 Hz, 1H), 7.60 (d, J=2.0 Hz, 1H), 7.45 (d, J=8.7 Hz, 1H), 7.33 (dd, J=8.6, 1.8 Hz, 1H), 7.16 (dd, J=8.7, 2.0 Hz, 1H), 7.02 (s, 1H), 3.66 (t, J=6.3 Hz, 2H), 3.54 (s, 2H), 3.48 (t, J=6.1 Hz, 2H), 2.97-2.74 (m, 6H), 2.06-1.76 (m, 2H). Mass calculated for (C₂₅H₂₆BrClN₆O₂+H)⁺559.1, found 559.4.

### Compound 129: 6-bromo-5′-chloro-N-(2-(4-(2-methoxyethyl)piperazin-1-yl)ethyl)-1H,1′H-2,2′-biindole-3-carboxamide

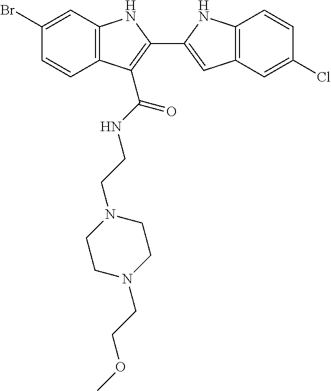 [SMILES: COCCN1CCN(CCNC(=O)c2c(-c3cc4cc(Cl)ccc4[nH]3)[nH]c3cc(Br)ccc23)CC1]

Prepared according to general methods XXXIII and XXXIV from intermediate 129-1 and intermediate 72-iii to yield 22 mg (23%). ¹H NMR (400 MHz, Methanol-d₄) δ 7.88 (d, J=8.6 Hz, 1H), 7.64 (d, J=1.8 Hz, 1H), 7.60 (d, J=2.0 Hz, 1H), 7.45 (d, J=8.7 Hz, 1H), 7.31 (dd, J=8.7, 1.8 Hz, 1H), 7.16 (dd, J=8.7, 2.0 Hz, 1H), 7.01 (s, 1H), 3.65 (t, J=6.4 Hz, 2H), 3.54 (t, J=5.5 Hz, 2H), 3.36 (s, 3H), 2.88-2.35 (m, 12H). Mass calculated for (C₂₆H₂₉BrClN₅O₂+H)⁺560.1, found 560.5.

### Compound 130: 6-bromo-5′-chloro-N-(2-(4-(2-methoxyethyl)-1,4-diazepan-1-yl)ethyl)-1H,1′H-2,2′-biindole-3-carboxamide

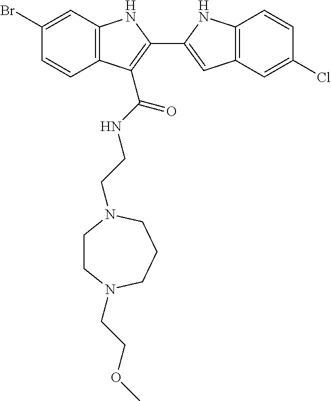 [SMILES: COCCN1CCCN(CCNC(=O)c2c(-c3cc4cc(Cl)ccc4[nH]3)[nH]c3cc(Br)ccc23)CC1]

Prepared according to general methods XXXIII and XXXIV from intermediate 130-i and intermediate 72-iii to yield 70 mg (45%). ¹H NMR (400 MHz, Methanol-d₄) δ 7.83 (d, J=8.6 Hz, 1H), 7.66 (d, J=1.7 Hz, 1H), 7.62 (d, J=2.0 Hz, 1H), 7.46 (d, J=8.7 Hz, 1H), 7.35 (dd, J=8.6, 1.8 Hz, 1H), 7.18 (dd, J=8.7, 2.1 Hz, 1H), 7.04 (d, J=0.9 Hz, 1H), 3.68 (t, J=6.1 Hz, 2H), 3.50 (t, J=5.3 Hz, 2H), 3.31 (s, 3H), 3.25-3.08 (m, 2H), 3.06-2.81 (m, 10H), 1.96 (p, J=5.8 Hz, 2H). Mass calculated for (C₂₇H₃₁BrClN₅O₂+H)⁺574.1, found 574.4.

### Compound 131: 6-bromo-N-(2-(1-carbamoylpiperidin-4-ylamino)ethyl)-5′-chloro-1H,1′H-2,2′-biindole-3-carboxamide

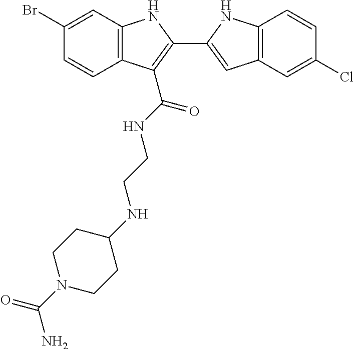 [SMILES: NC(=O)N1CCC(NCCNC(=O)c2c(-c3cc4cc(Cl)ccc4[nH]3)[nH]c3cc(Br)ccc23)CC1]

Prepared according to general methods XXXIII and XXXIV from intermediate 131-i and intermediate 72-iii to yield 33.2 mg (23%). ¹H NMR (400 MHz, Methanol-d₄) δ 7.82 (d, J=8.6 Hz, 1H), 7.66 (d, J=1.8 Hz, 1H), 7.62 (d, J=2.0 Hz, 1H), 7.45 (d, J=8.7 Hz, 1H), 7.35 (dd, J=8.6, 1.8 Hz, 1H), 7.18 (dd, J=8.7, 2.1 Hz, 1H), 7.05 (s, 1H), 4.09 (d, J=16.2 Hz, 2H), 3.78 (t, J=6.0 Hz, 2H), 3.23 (t, J=6.1 Hz, 2H), 2.96-2.76 (m, 2H), 2.06 (d, J=11.2 Hz, 1H), 1.45 (qd, J=12.0, 4.1 Hz, 2H). Mass calculated for (C₂₅H₂₆BrClN₆O₂+H)⁺559.1, found 559.5.

### Compound 132: 6-bromo-N-(2-(4-carbamoylpiperazin-1-yl)ethyl)-5′-chloro-1H,1′H-2,2′-biindole-3-carboxamide

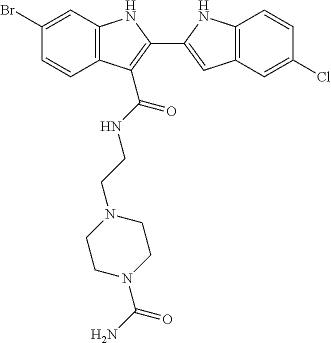 [SMILES: NC(=O)N1CCN(CCNC(=O)c2c(-c3cc4cc(Cl)ccc4[nH]3)[nH]c3cc(Br)ccc23)CC1]

Prepared according to general methods XXXIII and XXXIV from intermediate 132-i and intermediate 72-iii to yield 56.2 mg (39%) as fine tan crystals after flash purification and trituration with DCM. ¹H NMR (400 MHz, Methanol-d₄) δ 7.87 (d, J=8.6 Hz, 1H), 7.62 (d, J=1.7 Hz, 1H), 7.59 (d, J=2.0 Hz, 1H), 7.43 (d, J=8.7 Hz, 1H), 7.30 (dd, J=8.6, 1.8 Hz, 1H), 7.15 (dd, J=8.7, 2.1 Hz, 1H), 7.00 (d, J=0.9 Hz, 1H), 3.66 (t, J=6.3 Hz, 2H), 3.42 (t, J=5.0 Hz, 4H), 2.69 (t, J=6.3 Hz, 2H), 2.54 (t, J=5.0 Hz, 4H). Mass calculated for (C₂₄H₂₄BrClN₆O₂+H)⁺545.1, found 545.4.

### Compound 133: 6-Bromo-5′-chloro-N-(3-hydroxypropyl)-1H,1′H-[2,2′-biindole]-3-carboxamide

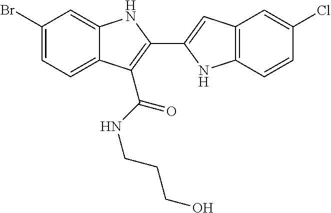 [SMILES: O=C(NCCCO)c1c(-c2cc3cc(Cl)ccc3[nH]2)[nH]c2cc(Br)ccc12]

Prepared according general method XXXIV from 3-aminopropan-1-ol and intermediate 72-iii (23 mg, 52%). ¹H NMR (400 MHz, DMSO-d₆) δ 12.58 (s, 1H), 12.28 (s, 1H), 8.31 (t, J=5.5 Hz, 1H), 7.79-7.69 (m, 2H), 7.66-7.59 (m, 2H), 7.32 (dd, J=8.6, 1.8 Hz, 1H), 7.20-7.12 (m, 2H), 4.59 (t, J=5.1 Hz, 1H), 3.56 (q, J=6.0 Hz, 2H), 3.47 (q, J=6.6 Hz, 2H), 1.79 (p, J=6.6 Hz, 2H). 13C NMR (101 MHz, DMSO-d₆) δ 166.11, 137.20, 135.08, 133.03, 131.58, 129.30, 125.43, 124.83, 124.01, 123.01, 122.59, 119.96, 115.98, 114.37, 114.10, 108.97, 101.70, 59.42, 37.68, 32.66. Mass calculated for (C₂₀H₁₇BrClN₃O₂−H)⁻ 444.0, found 444.0.

### Compound 134: 6-Bromo-5′-chloro-N-(2,2-dimethoxyethyl)-1H,1′H-[2,2′-biindole]-3-carboxamide

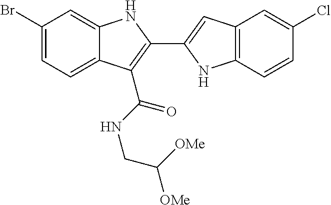 [SMILES: COC(CNC(=O)c1c(-c2cc3cc(Cl)ccc3[nH]2)[nH]c2cc(Br)ccc12)OC]

Prepared according general method XXXIV from 2,2-dimethoxyethan-1-amine and and intermediate 72-iii (77 mg, 64%). ¹H NMR (400 MHz, DMSO-d₆) δ 12.34 (s, 1H), 12.29 (s, 1H), 8.31 (t, J=5.8 Hz, 1H), 7.79-7.71 (m, 2H), 7.65-7.59 (m, 2H), 7.34 (dd, J=8.6, 1.8 Hz, 1H), 7.20-7.12 (m, 2H), 4.68 (t, J=5.6 Hz, 1H), 3.51 (t, J=5.8 Hz, 2H), 3.35 (s, 6H). Mass calculated for (C₂₁H₁₉BrClN₃O₃−H)⁻ 474.0, found 474.0.

### Compound 135: 6-Bromo-5′-chloro-N-(4,4-diethoxybutyl)-1H,1′H-[2,2′-biindole]-3-carboxamide

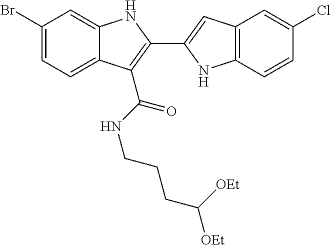 [SMILES: CCOC(CCCNC(=O)c1c(-c2cc3cc(Cl)ccc3[nH]2)[nH]c2cc(Br)ccc12)OCC]

Prepared according general method XXXIV from 4,4-diethoxybutan-1-amine and intermediate 72-iii (87 mg, 64%). ¹H NMR (400 MHz, DMSO-d₆) δ 12.53 (s, 1H), 12.28 (s, 1H), 8.34 (t, J=5.6 Hz, 1H), 7.77-7.68 (m, 2H), 7.65-7.56 (m, 2H), 7.32 (dd, J=8.7, 1.8 Hz, 1H), 7.21-7.11 (m, 2H), 4.53 (d, J=4.6 Hz, 1H), 3.56 (dq, J=9.5, 7.0 Hz, 4H), 1.50 (dt, J=14.2, 7.1 Hz, 2H), 1.10 (t, J=7.0 Hz, 6H), 1.03 (d, J=6.3 Hz, 2H), 0.88 (q, J=7.4 Hz, 2H). Mass calculated for (C₂₅H₂₇BrClN₃O₃−H)⁻ 530.1, found 530.1.

### Synthetic Scheme for Compound 136: 6-Bromo-5′-chloro-N-(2-(methylamino)ethyl)-1H,1′H-[2,2′-biindole]-3-carboxamide

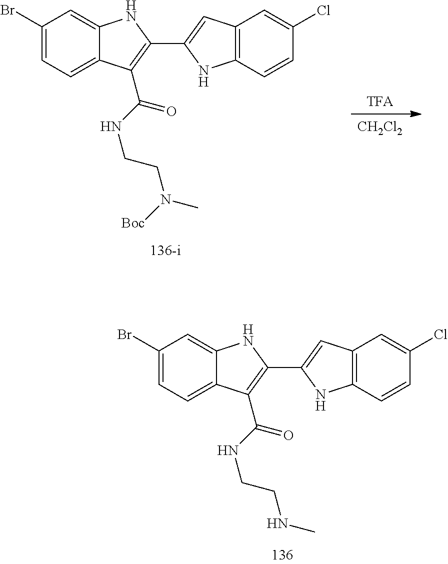 [SMILES: CN(CCNC(=O)c1c(-c2cc3cc(Cl)ccc3[nH]2)[nH]c2cc(Br)ccc12)C(=O)OC(C)(C)C ; "CH2Cl2" ; CNCCNC(=O)c1c(-c2cc3cc(Cl)ccc3[nH]2)[nH]c2cc(Br)ccc12 ; "TFA"]

Intermediate 136-i was prepared according general method XXXIV from tert-butyl (2-aminoethyl)(methyl)carbamate and intermediate 72-iii (109 mg, 99%). TFA (65 equiv) was added to a solution of intermediate 136-i in CH₂Cl₂ and stirred for 1 h at ambient temperature. The mixture was concentrated in vacuo and co-evaporated with CH₂Cl₂ (3×). The resulting off-white solid was collected and rinsed with CH₂Cl₂ to afford desired secondary amine 136 (13 mg) in 15% yield. ¹H NMR (400 MHz, DMSO-d₆) δ 12.62-12.13 (m, 2H), 8.30 (d, J=5.9 Hz, 1H), 7.83 (d, J=8.6 Hz, 1H), 7.74 (d, J=1.9 Hz, 1H), 7.65 (s, 1H), 7.58 (d, J=8.6 Hz, 1H), 7.33 (d, J=8.6 Hz, 1H), 7.18 (q, J=3.9, 2.9 Hz, 2H), 4.11 (s, 1H), 3.61 (q, J=5.8 Hz, 3H), 3.18 (s, 2H), 3.02 (t, J=6.2 Hz, 2H). Mass calculated for (C₂₀H₁₈BrClN₄O−H)⁻ 443.1, found 443.0.

### Synthetic Scheme for Compound 137: 6-Bromo-5′-chloro-N-(2-(ethylamino)ethyl)-1H,1′H-[2,2′-biindole]-3-carboxamide

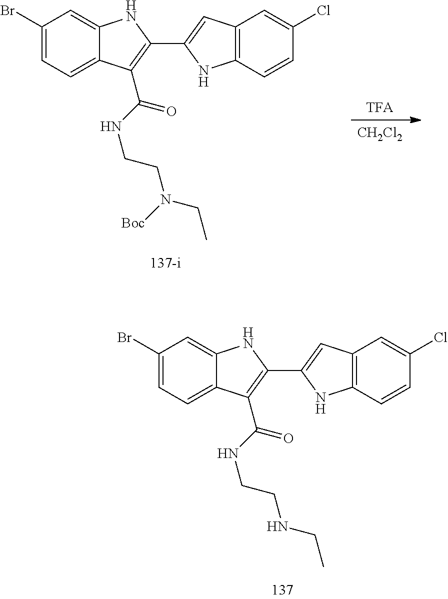 [SMILES: CCN(CCNC(=O)c1c(-c2cc3cc(Cl)ccc3[nH]2)[nH]c2cc(Br)ccc12)C(=O)OC(C)(C)C ; "CH2Cl2" ; CCNCCNC(=O)c1c(-c2cc3cc(Cl)ccc3[nH]2)[nH]c2cc(Br)ccc12 ; "TFA"]

Intermediate 137-i was prepared according general method XXXIV from tert-butyl (2-aminoethyl)(ethyl)carbamate (97 mg, 85%). TFA (75 equiv) was added to a solution of intermediate 137-i in CH₂Cl₂ and stirred for 1 h at ambient temperature. The mixture was concentrated in vacuo and co-evaporated with CH₂Cl₂ (3×). The resulting white solid was collected and rinsed with CH₂Cl₂ to afford desired secondary amine 137 (30 mg) in 30% yield. ¹H NMR (400 MHz, DMSO-d₆) δ 12.46 (s, 1H), 12.38 (s, 1H), 8.37 (s, 1H), 7.84 (d, J=8.6 Hz, 1H), 7.75 (d, J=2.0 Hz, 1H), 7.65 (d, J=1.8 Hz, 1H), 7.56 (d, J=8.7 Hz, 1H), 7.36 (dd, J=8.6, 1.8 Hz, 1H), 7.25-7.15 (m, 2H), 3.69 (q, J=6.0 Hz, 2H), 3.19 (t, J=6.2 Hz, 3H), 3.03 (q, J=6.6 Hz, 2H), 1.19 (t, J=7.2 Hz, 3H). Mass calculated for (C₂₁H₂₀BrClN₄O−H)⁻ 457.1, found 457.0.

### Compound 138: 6-Bromo-N-(2-(tert-butylamino)ethyl)-5′-chloro-1H,1′H-[2,2′-biindole]-3-carboxamide

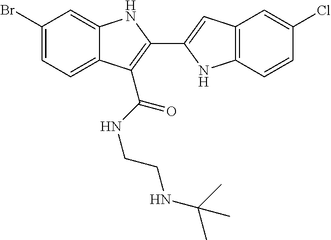 [SMILES: CC(C)(C)NCCNC(=O)c1c(-c2cc3cc(Cl)ccc3[nH]2)[nH]c2cc(Br)ccc12]

Prepared according general method XXXIV from N¹-(tert-butyl)ethane-1,2-diamine and and intermediate 72-iii (92 mg, 73%). ¹H NMR (400 MHz, DMSO-d₆) δ 12.36 (s, 2H), 8.22 (s, 1H), 7.87 (d, J=8.6 Hz, 1H), 7.73 (d, J=2.0 Hz, 1H), 7.64 (d, J=1.8 Hz, 1H), 7.60 (d, J=8.7 Hz, 1H), 7.32 (dd, J=8.6, 1.8 Hz, 1H), 7.21-7.13 (m, 2H), 4.12 (s, 1H), 3.50 (d, J=6.0 Hz, 2H), 2.83 (d, J=6.7 Hz, 2H), 1.10 (s, 9H). Mass calculated for (C₂₃H₂₄BrClN₄O−H)⁻ 485.1, found 485.0.

### Compound for 139: 6-Bromo-5′-chloro-N-(2-(4-methyl-2-phenylpiperazin-1-yl)ethyl)-1H,1′H-[2,2′-biindole]-3-carboxamide

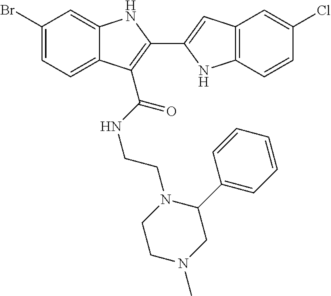 [SMILES: CN1CCN(CCNC(=O)c2c(-c3cc4cc(Cl)ccc4[nH]3)[nH]c3cc(Br)ccc23)C(c2ccccc2)C1]

Prepared according general method XXXIV from 2-(4-methyl-2-phenylpiperazin-1-yl)ethan-1-amine and intermediate 72-iii (90 mg, 60%). ¹H NMR (400 MHz, DMSO-d₆) δ 12.51 (s, 1H), 12.30 (s, 1H), 8.02 (t, J=5.6 Hz, 1H), 7.88 (d, J=8.6 Hz, 1H), 7.73 (d, J=2.0 Hz, 1H), 7.64 (d, J=1.8 Hz, 1H), 7.55 (d, J=8.7 Hz, 1H), 7.33 (dd, J=8.6, 1.8 Hz, 1H), 7.30-7.25 (m, 2H), 7.21-7.05 (m, 5H), 3.44 (dt, J=10.0, 3.9 Hz, 2H), 3.27 (td, J=10.0, 2.7 Hz, 2H), 3.18 (d, J=4.5 Hz, 1H), 2.85-2.78 (m, 1H), 2.72 (dt, J=12.0, 8.2 Hz, 1H), 2.60 (dt, J=11.0, 2.6 Hz, 1H), 2.29 (td, J=11.1, 2.6 Hz, 1H), 2.15 (s, 3H), 2.05 (dq, J=12.5, 4.5, 4.0 Hz, 1H), 1.84 (t, J=10.7 Hz, 1H). ¹³C NMR (101 MHz, DMSO-d₆) δ 165.80, 142.22, 137.21, 135.10, 133.04, 131.48, 129.30, 128.66, 128.11, 127.61, 125.51, 124.81, 123.89, 123.00, 122.80, 120.01, 116.02, 114.44, 114.09, 108.86, 101.78, 67.09, 64.23, 55.31, 53.50, 51.51, 45.93, 36.93. Mass calculated for (C₃₀H₂₉BrClN₅O−H)⁻ 588.1, found 588.0.

### Synthesis of Compound 140: 6′-Bromo-5-chloro-6-methoxy-1H,1′H-2,2′-biindole

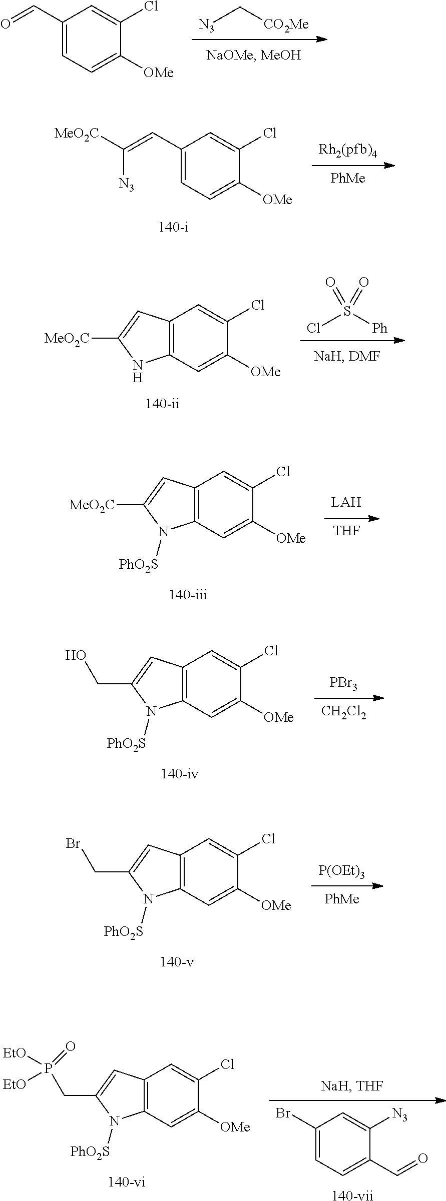

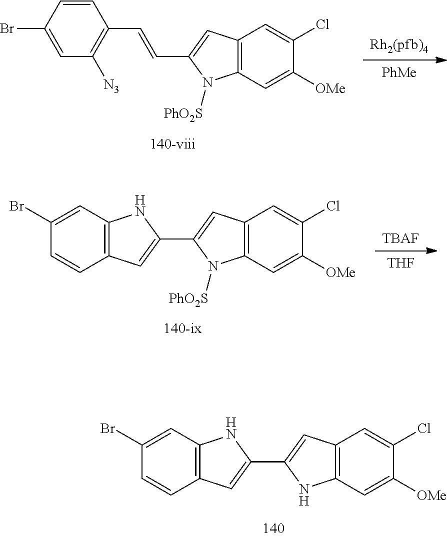

### Compound 140-i: Methyl (Z)-2-azido-3-(3-chloro-4-methoxyphenyl)acrylate

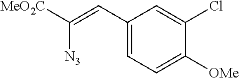 [SMILES: COC(=O)/C(=C/c1ccc(OC)c(Cl)c1)N=[N+]=[N-]]

Vinyl azide 140-i was prepared according to literature procedures (J. Am. Chem. Soc. 2007, 129, 7500).

### Compound 140-ii: Methyl 5-chloro-6-methoxy-1H-indole-2-carboxylate

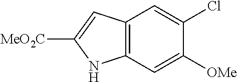 [SMILES: COC(=O)c1cc2cc(Cl)c(OC)cc2[nH]1]

Indole 140-ii was prepared according to literature procedures (J. Am. Chem. Soc. 2007, 129, 7500).

### Compound 140-iii: Methyl 5-chloro-6-methoxy-1-(phenylsulfonyl)-1H-indole-2-carboxylate

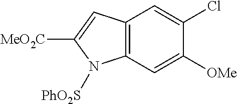

60% Sodium hydride (1.8 equiv) was added in one portion to a stirring solution of indole 140-ii in DMF under N₂. The milky mixture was stirred for 10 minutes then benzylsulfonyl chloride (1.8 equiv) was added. After stirring at ambient temperature for 21 h, the yellow mixture was diluted with EtOAc, washed with H₂O (1×) and brine (1×), dried over MgSO₄, filtered, concentrated in vacuo and purified by column chromatography with 10-80% Et₂O/hexanes to afford the desired protected indole 140-iii as a white solid in 78% yield. ¹H NMR (400 MHz, CDCl₃) δ 8.02-7.94 (m, 2H), 7.77 (s, 1H), 7.61 (d, J=15.2 Hz, 2H), 7.52 (dd, J=8.5, 7.1 Hz, 2H), 7.15 (s, 1H), 4.04 (s, 3H), 3.92 (s, 3H). Mass calculated for (C₁₇H₁₄ClNO₅S+H)⁺380.0, found 380.4.

### Compound 140-iv: (5-Chloro-6-methoxy-1-(phenylsulfonyl)-1H-indol-2-yl)methanol

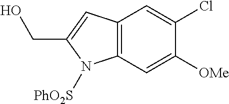

Lithium aluminum hydride (2.4 equiv) was added in one portion to a cold (−5° C.) stirring solution of protected indole 140-iii in THF under N₂. After 90 minutes, the cold mixture was quenched with H₂O and 5M NaOH. The mixture was warmed with ambient temperature and passed through a bed of Celite. The filtrate was concentrated in vacuo, purified by column chromatography with 15-60% EtOAc/hexanes and triturated with Et₂O to afford the desired alcohol 140-iv as a white solid in 57% yield. ¹H NMR (400 MHz, CDCl₃) δ 7.85-7.78 (m, 2H), 7.70 (s, 1H), 7.64-7.57 (m, 1H), 7.53-7.45 (m, 3H), 6.57 (s, 1H), 4.87 (s, 2H), 3.99 (s, 3H), 3.01 (s, 1H). Mass calculated for (C₁₆H₁₄ClNO₄S+H)⁺352.0, found 334.2 (M−OH).

### Compound 140-v: 2-(Bromomethyl)-5-chloro-6-methoxy-1-(phenylsulfonyl)-1H-indole

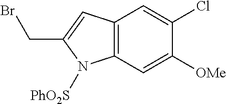

Phosphorus tribromide (1.3 equiv) was added in one portion to a cold (0° C.) stirring suspension of alcohol 140-iv in CH₂Cl₂. After stirring at ambient temperature for 90 minutes, the pale yellow mixture was cooled back down to 0° C. and quenched with saturated aq NaHCO₃. The organic layer was washed with H₂O (1×) and brine (1×), dried over MgSO₄, filtered, concentrated in vacuo to afford the desired bromide 140-v as a white solid in 99% yield. ¹H NMR (400 MHz, CDCl₃) δ 7.88-7.83 (m, 2H), 7.71 (s, 1H), 7.64-7.58 (m, 1H), 7.51-7.45 (m, 3H), 6.73 (s, 1H), 4.97 (s, 2H), 4.00 (s, 3H).

### Compound 140-vi: Diethyl ((5-chloro-6-methoxy-1-(phenylsulfonyl)-1H-indol-2-yl)methyl)phosphonate

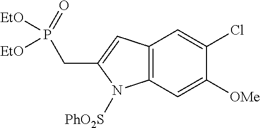

A mixture of bromide 140-v, triethyl phosphite (3.7 equiv) and toluene was stirred at 125° C. for 80 minutes. The mixture was cooled to ambient temperature, diluted with EtOAc, concentrated in vacuo and co-evaporated with EtOAc (3×) to afford an orange oil. The oily crude was triturated with a Et₂O/hexanes mixture to afford the desired phosphonate 140-vi as an off-white solid in 92% yield. ¹H NMR (400 MHz, CDCl₃) δ 7.77 (s, 1H), 7.76-7.70 (m, 2H), 7.61-7.55 (m, 1H), 7.49-7.42 (m, 3H), 6.75 (d, J=3.3 Hz, 1H), 4.20-4.08 (m, 4H), 4.00 (s, 3H), 3.72 (d, J=21.9 Hz, 2H), 1.32 (t, J=7.1 Hz, 6H). 13C NMR (101 MHz, CDCl₃) δ 152.78, 138.30, 136.33, 134.09, 130.89, 129.38, 126.23, 123.45, 121.42, 120.01, 111.88, 111.81, 99.33, 62.57, 62.50, 56.61, 26.93, 25.52, 16.43, 16.38. ³¹P NMR (162 MHz, CDCl₃) δ 23.52. Mass calculated for (C₂₀H₂₃ClNO₆PS+H)⁺472.1, found 472.3.

### Compound 140-vii: 2-Azido-4-bromobenzaldehyde

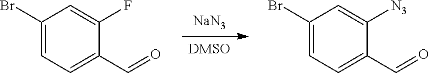

A mixture of 4-bromo-2-fluorobenzaldehyde, sodium azide (3.1 equiv) and DMSO was stirred at 60° C. for 24 h. The mixture was diluted with TBME, washed with H₂O (1×), saturated aq NH₄Cl (1×), H₂O (2×) and brine (1×). The organic layer was dried over MgSO₄, filtered, concentrated in vacuo to afford the desired aryl azide 140-vii as a white solid in 50% yield. ¹H NMR (400 MHz, CDCl₃) δ 10.27 (s, 1H), 7.93 (d, J=8.4 Hz, 1H), 6.93 (ddd, J=8.4, 2.1, 0.8 Hz, 1H), 6.85 (d, J=2.0 Hz, 1H). Mass calculated for (C₇H₄BrN₃O+H)⁺226.0, found 198.2 (M−N₂+H).

### Compound 140-viii: (E)-2-(2-azido-4-bromostyryl)-5-chloro-6-methoxy-1-(phenylsulfonyl)-1H-indole

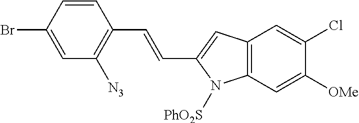

60% Sodium hydride (1.3 equiv) was added in one portion to a cold (0° C.) stirring solution of phosphonate 140-vi in THF under N₂. The green cloudy mixture was stirred cold for 30 minutes then aryl azide 140-vii (1.1 equiv) was added. After stirring at ambient temperature for 50 minutes, the dark green mixture was quenched with H₂O. The resulting bright red mixture was extracted EtOAc (Ix), washed with H₂O (1×) and brine (1×), dried over MgSO₄, filtered, concentrated in vacuo and purified by column chromatography with 5-20% Et₂O/hexanes to afford the desired vinyl indole 140-viii as a yellow solid in 61% yield. H NMR (400 MHz, CDCl₃) δ 7.86 (s, 1H), 7.77 (d, J=16.2 Hz, 1H), 7.72-7.67 (m, 2H), 7.62-7.52 (m, 2H), 7.46 (s, 1H), 7.40 (t, J=7.9 Hz, 2H), 7.34 (d, J=7.3 Hz, 2H), 7.14 (d, J=16.3 Hz, 1H), 6.79 (s, 1H), 4.05 (s, 3H).

### Compound 140-ix: 6′-Bromo-5-chloro-6-methoxy-1-(phenylsulfonyl)-1H,1′H-2,2′-biindole

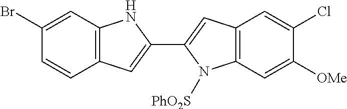

A mixture of vinyl indole 140-viii, rhodium(II) perfluorobutyrate dimer (3.3 mol %) and toluene was stirred at 80° C. under N₂ for 16 h. The mixture was cooled to ambient temperature, concentrated in vacuo and purified by column chromatography with 5-30% Et₂O/hexanes to afford the desired protected biindole 140-ix as a white solid in 71% yield. H NMR (400 MHz, CDCl₃) δ 8.91 (s, 1H), 7.99 (s, 1H), 7.69-7.63 (m, 1H), 7.54-7.47 (m, 3H), 7.44-7.37 (m, 2H), 7.30-7.26 (m, 3H), 6.71 (s, 1H), 6.61 (dd, J=2.1, 1.0 Hz, 1H), 4.09 (s, 3H). Mass calculated for (C₂₃H₁₆BrClN₂O₃S+H)⁺515.0, found 515.1.

### Compound 140: 6′-Bromo-5-chloro-6-methoxy-1H,1′H-2,2′-biindole

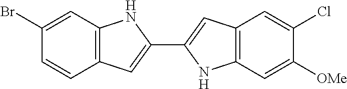 [SMILES: COc1cc2[nH]c(-c3cc4ccc(Br)cc4[nH]3)cc2cc1Cl]

A 1M solution of tetrabutylammonium fluoride (5.3 equiv) in THF was added to a stirring solution of the protected biindole 140-ix in THF under N₂. The resulting bright yellow mixture was stirred at 60° C. for 3 h, cooled to ambient temperature, quenched with H₂O, extracted with EtOAc (1×), washed with H₂O (1×) and brine (1×), dried over MgSO₄, filtered, concentrated in vacuo, purified by column chromatography with 15-60% Et₂O/hexanes and triturated with a CH₂Cl₂/hexanes mixture to afford the desired biindole as a maroon solid in 64% yield. ¹H NMR (400 MHz, DMSO-d₆) δ 11.69 (d, J=2.1 Hz, 1H), 11.66-11.57 (m, 1H), 7.65 (s, 1H), 7.58-7.49 (m, 2H), 7.14 (dd, J=8.4, 1.8 Hz, 1H), 7.04 (s, 1H), 6.87 (dd, J=15.5, 2.0 Hz, 2H), 3.90 (s, 3H). ¹³C NMR (101 MHz, DMSO-d₆) δ 151.24, 138.22, 136.82, 132.69, 131.19, 127.97, 123.02, 122.77, 122.13, 121.16, 115.30, 114.56, 113.88, 98.90, 98.67, 95.27, 56.57. Mass calculated for (C₁₇H₁₂BrClN₂O+H)⁺375.0, found 375.2.

### Compound 141: 1-(5-bromobenzo[d]thiazol-2-yl)-3-(4-chlorophenyl)urea

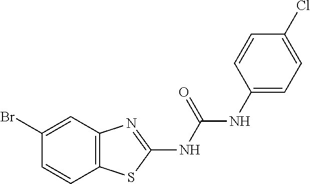 [SMILES: O=C(Nc1ccc(Cl)cc1)Nc1nc2cc(Br)ccc2s1]

Prepared according to general method XXVIII from intermediate 99-i and 4-chlorophenylisocyanate and recovered as pure precipitate that was washed with toluene but required no flash purification (23.4 mg, 19%). ¹H NMR (400 MHz, DMSO-d₆) δ 9.23 (s, 1H), 9.03 (s, 1H), 8.10 (d, J=2.3 Hz, 1H), 7.75 (d, J=8.7 Hz, 1H), 7.56-7.42 (m, 3H), 7.36 (d, J=8.9 Hz, 2H). Mass calculated for (C₁₄H₉BrClN₃OS+H)⁺384.0, found 384.4.

### Compound 142: 6-bromo-5′-chloro-N-(2-(pyridin-4-yl)ethyl)-1H,1′H-[2,2′-biindole]-3-carboxamide

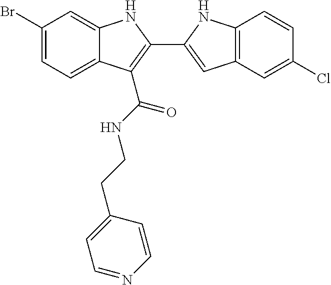 [SMILES: O=C(NCCc1ccncc1)c1c(-c2cc3cc(Cl)ccc3[nH]2)[nH]c2cc(Br)ccc12]

Prepared according to general method XXXIV from intermediate 72-iii and 4-(2-ethylamino)-pyridine with purification by concentration, then direct flash purification with a gradient of 2-30% MeOH/CH₂Cl₂ then trituration with CH₂Cl₂ to yield the title compound (22.8 mg, 36%) as a tan powder. ¹H NMR (400 MHz, DMSO-d₆) δ 12.40 (s, 1H), 12.32 (s, 1H), 8.52-8.45 (m, 2H), 8.39 (t, J=5.6 Hz, 1H), 7.72 (d, J=2.0 Hz, 1H), 7.67-7.55 (m, 2H), 7.47 (d, J=8.6 Hz, 1H), 7.34 (d, J=6.0 Hz, 2H), 7.24 (dd, J=8.6, 1.8 Hz, 1H), 7.17 (dd, J=8.7, 2.1 Hz, 1H), 7.13 (d, J=1.8 Hz, 1H), 3.71 (q, J=6.7 Hz, 2H), 2.99 (t, J=7.0 Hz, 2H). Mass calculated for (C₂₄H₁₈BrClN₄O+H)⁺495.0, found 495.3.

### Intermediate 143-i: Tert-butyl 4-(2-(6-bromo-5′-chloro-1H, 1′H-[2,2′-biindole]-3-carboxamido)ethyl)-3-oxopiperazine-1-carboxylate

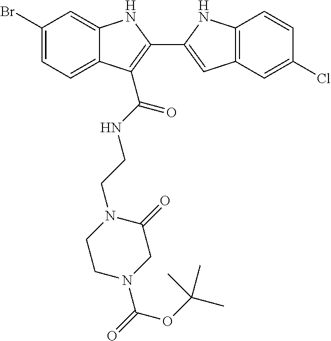 [SMILES: CC(C)(C)OC(=O)N1CCN(CCNC(=O)c2c(-c3cc4cc(Cl)ccc4[nH]3)[nH]c3cc(Br)ccc23)C(=O)C1]

Prepared according to general method XXXIV from intermediate 72-iii and tert-butyl 4-(2-aminoethyl)-3-oxopiperazine-1-carboxylate (prepared according to Crawford, J. J., et al. PCT Int. Appl. 2015011252) with an EtOAc/aqueous workup and purification by flash (50-100% EtOAc/Hex) to yield the title compound as a clear film (60 mg, 76%). ¹H NMR (400 MHz, Chloroform-d) δ 12.44 (s, 1H), 8.84 (s, 1H), 7.75 (d, J=8.6 Hz, 1H), 7.62 (s, 1H), 7.58 (s, 1H), 7.44 (d, J=8.7 Hz, 1H), 7.39 (d, J=9.0 Hz, 1H), 7.21 (dd, J=8.7, 2.0 Hz, 1H), 7.12 (s, 1H), 6.81 (s, 1H), 4.05 (s, 2H), 3.81 (s, 4H), 3.71 (s, 2H), 3.54 (s, 2H), 1.45 (s, 9H). Mass calculated for (C₂₈H₂₉BrClN₅O₄+H)⁺ 616.1, found 616.4.

### Compound 143: 6-bromo-5′-chloro-N-(2-(2-oxopiperazin-1-yl)ethyl)-1H,1′H-[2,2′-biindole]-3-carboxamide

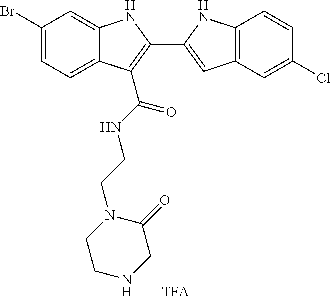 [SMILES: O=C(NCCN1CCNCC1=O)c1c(-c2cc3cc(Cl)ccc3[nH]2)[nH]c2cc(Br)ccc12]

Prepared according to general method XVI from 143-i to yield the title compound as a tan powder (16.5 mg, 32%) after precipitation from MeOH/CH₂Cl₂ (1:20) with Et₂O at 0° C. ¹H NMR (400 MHz, DMSO-d₆) δ 12.47 (s, 1H), 12.32 (s, 1H), 8.87 (s, 2H), 8.29 (t, J=5.4 Hz, 1H), 7.79 (d, J=8.6 Hz, 1H), 7.74 (d, J=1.9 Hz, 1H), 7.63 (s, 1H), 7.61 (d, J=8.7 Hz, 1H), 7.32 (d, J=8.4 Hz, 1H), 7.22-7.11 (m, 2H), 3.71-3.56 (m, 8H), 3.41-3.36 (m, 2H). Mass calculated for (C₂₃H₂₁BrClN₅O₂+H)⁺516.1, found 516.4.

### General Method XXXV

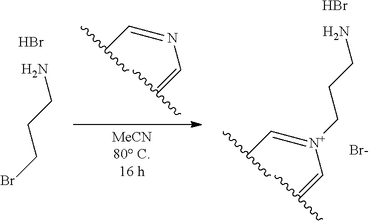

3-Bromopropylamine hydrobromide (1.0 eq) was taken up in acetonitrile (10 mL/g), then the desired pyridine nucleophile (2.0 eq) was added and heated to 80° C. in a sealed vial overnight. The product crashed out as a solid precipitate, or a syrup that solidified upon cooling. Filtration and washing with ethanol yielded the title compounds in >90% purity.

### Intermediate 144-i

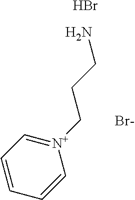

Prepared according to general method XXXV with pyridine to yield a white solid (290 mg, 43%). ¹H NMR (400 MHz, DMSO-d₆) δ 9.16 (d, J=6.0 Hz, 2H), 8.66 (t, J=7.8 Hz, 1H), 8.22 (d, J=6.8 Hz, 2H), 7.93 (s, 3H), 4.75 (t, J=7.2 Hz, 2H), 2.95-2.83 (m, 2H), 2.25 (p, J=7.3 Hz, 2H).

### Compound 144: 1-(3-(6-bromo-5′-chloro-1H,1′H-[2,2′-biindole]-3-carboxamido)propyl)pyridin-1-ium

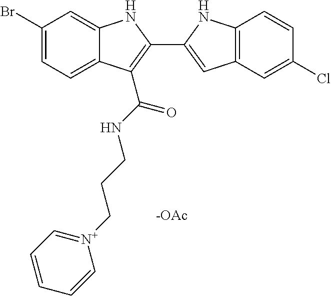 [SMILES: O=C(NCCC[n+]1ccccc1)c1c(-c2cc3cc(Cl)ccc3[nH]2)[nH]c2cc(Br)ccc12]

Prepared according to general method XXXIV from intermediates 72-iii and 144-i with purification by directly loading onto a flash column, running 1-70% MeOH/CH₂Cl₂ then, 30-95% (5% AcOH/MeOH)/CH₂Cl₂ to give the title compound as a clear film (60 mg, 82%). ¹H NMR (400 MHz, DMSO-d₆) δ 9.15 (d, J=5.5 Hz, 2H), 8.59 (t, J=7.8 Hz, 1H), 8.14 (t, J=7.0 Hz, 2H), 7.71 (d, J=8.5 Hz, 1H), 7.64 (d, J=1.8 Hz, 1H), 7.58 (d, J=2.0 Hz, 1H), 7.44 (d, J=8.6 Hz, 1H), 7.19 (dd, J=8.5, 1.8 Hz, 1H), 7.17 (s, 1H), 7.05 (d, J=8.7 Hz, 1H), 4.74 (t, J=7.1 Hz, 2H), 3.43 (t, J=6.6 Hz, 2H), 2.30 (p, J=6.8 Hz, 2H). Mass calculated for (C₂₅H₂₁BrClN₄O)⁺509.1, found 509.1.

### Intermediate 145-i: 1-(3-aminopropyl)-4-(dimethylamino)pyridin-1-ium

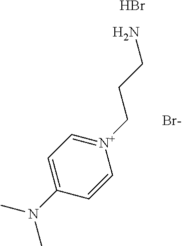

Prepared according to general method XXXV with 4-dimethylaminopyridine to yield a white solid (621 mg, 80%). ¹H NMR (400 MHz, DMSO-d₆) δ 8.35 (d, J=7.7 Hz, 2H), 7.97 (s, 3H), 7.10 (d, J=7.8 Hz, 2H), 4.28 (t, J=7.0 Hz, 2H), 3.21 (s, 6H), 2.87-2.71 (m, 2H), 2.07 (p, J=7.5 Hz, 2H).

### Compound 145: 1-(3-(6-bromo-5′-chloro-1H, 1′H-[2,2′-biindole]-3-carboxamido)propyl)-4-(dimethylamino)pyridin-1-ium

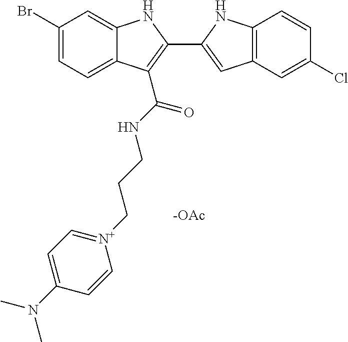 [SMILES: CN(C)c1cc[n+](CCCNC(=O)c2c(-c3cc4cc(Cl)ccc4[nH]3)[nH]c3cc(Br)ccc23)cc1]

Prepared according to general method XXXIV from intermediates 72-iii and 145-i with purification by directly loading onto a flash column, running 0-60% MeOH/CH₂Cl₂ then, 60-95% (5% AcOH/MeOH)/CH₂Cl₂, followed by precipitation from MeOH/CH₂Cl₂ with Et₂O at 0° C. to give the title compound as a white powder (23.1 mg, 49%). ¹H NMR (400 MHz, DMSO-d₆) δ 8.33 (d, J=7.2 Hz, 2H), 7.69 (s, 1H), 7.63 (s, 1H), 7.56 (s, 1H), 7.43 (d, J=8.6 Hz, 1H), 7.18 (d, J=8.4 Hz, 1H), 7.14 (s, 1H), 7.04 (d, J=8.7 Hz, 1H), 6.98 (d, J=7.1 Hz, 2H), 4.29 (t, J=6.9 Hz, 2H), 3.40-3.34 (m, 2H), 3.14 (s, 6H), 2.18-2.09 (m, 2H). Mass calculated for (C₂₇H₂₆BrClN₅O)⁺552.1, found 552.4.

### Intermediate 146-i: 2-(3-aminopropyl)isoquinolin-2-ium

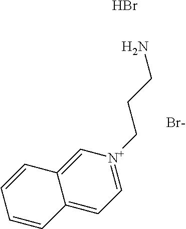 [SMILES: Br ; NCCC[n+]1ccc2ccccc2c1 ; [Br-]]

Prepared according to general method XXXV with isoquinoline to yield a light beige powder (782 mg, 98%). ¹H NMR (400 MHz, DMSO-d₆) δ 10.17 (s, 1H), 8.84 (d, J=7.9 Hz, 1H), 8.66 (d, J=6.8 Hz, 1H), 8.52 (d, J=8.3 Hz, 1H), 8.39 (d, J=8.3 Hz, 1H), 8.30 (t, J=7.6 Hz, 1H), 8.11 (t, J=7.6 Hz, 1H), 7.89 (s, 3H), 4.84 (t, J=7.0 Hz, 2H), 2.93 (q, J=7.6, 7.0 Hz, 2H), 2.34 (p, J=7.1 Hz, 2H).

### Compound 146: 2-(3-(6-bromo-5′-chloro-1H,1′H-[2,2′-biindole]-3-carboxamido)propyl)isoquinolin-2-ium

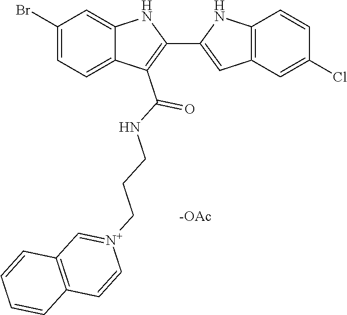 [SMILES: O=C(NCCC[n+]1ccc2ccccc2c1)c1c(-c2cc3cc(Cl)ccc3[nH]2)[nH]c2cc(Br)ccc12]

Prepared according to general method XXXIV from intermediates 146-i and 72-iii with purification by directly loading onto a flash column, running 0-60% MeOH/CH₂Cl₂ then, 60-95% (5% AcOH/MeOH)/CH₂Cl₂, followed by precipitation from MeOH/CH₂Cl₂ with Et₂O at 0° C. to give the title compound as a white powder (24.8 mg, 53%). ¹H NMR (400 MHz, DMSO-d₆) δ 10.16 (s, 1H), 8.85 (d, J=6.8 Hz, 1H), 8.56 (d, J=6.7 Hz, 1H), 8.43 (d, J=8.3 Hz, 1H), 8.32 (d, J=8.4 Hz, 1H), 8.24 (t, J=7.7 Hz, 1H), 8.04 (t, J=7.7 Hz, 1H), 7.69 (s, 1H), 7.63 (s, 1H), 7.56 (s, 1H), 7.43 (d, J=8.6 Hz, 1H), 7.27-7.11 (m, 2H), 7.04 (d, J=9.2 Hz, 1H), 4.86 (t, J=7.2 Hz, 2H), 3.47-3.34 (m, 2H), 2.46-2.33 (m, 2H). Mass calculated for (C₂₉H₂₃BrClN₄O)⁺559.1, found 559.4.

### Intermediate 147-i: 3-amino-N,N,N-trimethylpropan-1-aminium

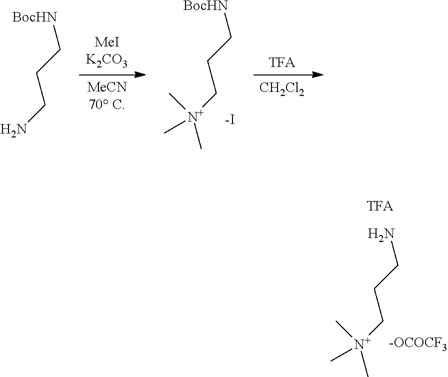

To a stirring solution of tert-butyl (3-aminopropyl)carbamate (320 mg, 1.84 mmol) in acetonitrile (5 mL) was added potassium carbonate (1.02 g, 7.36 mmol, 4.0 eq) and methyl iodide (0.57 mL, 9.20 mmol, 5.0 eq) and the mixture was heated to 70° C. in a sealed vial. After 16 h, the reaction was allowed to cool to rt. 5 mL of water was added, dissolving all precipitate and giving two layers. The top layer was isolated and concentrated, then the residue was taken up in CH₂Cl₂, filtered, and the filtrate concentrated to give 596 mg (94%) of white solid. ¹H NMR (400 MHz, Chloroform-d) δ 5.40 (s, 1H), 3.75 (t, J=8.2 Hz, 2H), 3.45 (s, 9H), 3.30 (q, J=6.5 Hz, 2H), 2.12 (p, J=8.2 Hz, 2H), 1.45 (s, 9H). Mass calculated for (C₁₁H₂₅N₂O₂)⁺217.2, found 217.6.

The above isolated intermediate was suspended in CH₂Cl₂ (20 mL) and treated with trifluoroacetic acid (5 mL). After 2 h, the solution was concentrated and co-evaporated once with CH₂Cl₂ to yield a semisolid oil. ¹H NMR showed the oil to be mostly product, but it could be triturated to give about 80 mg (13%) of the title compound as a white solid. ¹H NMR (400 MHz, Deuterium Oxide) δ 3.41-3.24 (m, 2H), 3.12-3.02 (m, 9H), 2.98 (t, J=7.8 Hz, 2H), 2.10 (p, J=8.1 Hz, 2H).

### Compound 147: 3-(6-bromo-5′-chloro-1H,1′H-[2,2′-biindole]-3-carboxamido)-N,N,N-trimethylpropan-1-aminium

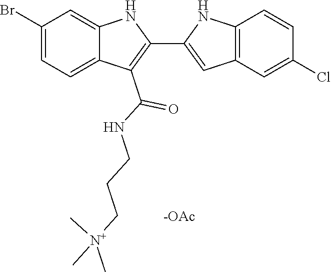 [SMILES: C[N+](C)(C)CCCNC(=O)c1c(-c2cc3cc(Cl)ccc3[nH]2)[nH]c2cc(Br)ccc12]

Prepared according to general method XXXV from intermediates 72-iii and 147-i and purified direct loading into a flash purification 2-95% MeOH/CH₂Cl₂, then 95% (1.5% AcOH/MeOH)/CH₂Cl₂ followed by precipitation from MeOH/CH₂Cl₂ (1:7) with Et₂O at 0° C. to give the title compound as a yellow powder (22.1 mg, 53%). ¹H NMR (400 MHz, DMSO-d₆) δ 7.69 (d, J=8.5 Hz, 1H), 7.66 (d, J=1.8 Hz, 1H), 7.61 (d, J=2.0 Hz, 1H), 7.47 (d, J=8.6 Hz, 1H), 7.23 (dd, J=8.5, 1.8 Hz, 1H), 7.20 (s, 1H), 7.09 (dd, J=8.6, 2.1 Hz, 1H), 3.45-3.36 (m, 4H), 3.06 (s, 9H), 2.04 (p, J=7.1, 6.7 Hz, 2H). Mass calculated for (C₂₃H₂₅BrClN₄O)⁺489.1, found 489.4.

### General Method XXXVI

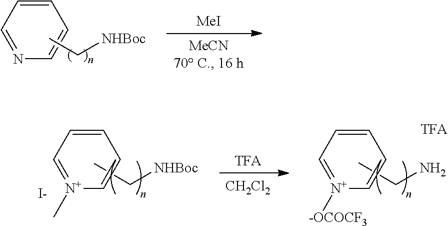

To a stirred solution of N-(tert-butoxycarbonyl)-(aminoalkyl)pyridine in acetonitrile (0.2-0.5 M) was added methyl iodide (1.5 eq), and the mixture was heated to 70° C. After 10 min, the solution turned bright yellow. After 16 h, the reaction was cooled and concentrated to yield methylated intermediate.

This pure intermediate was treated with TFA (3 mL) in DCM (5 mL). When the deprotection was complete after a few hours, the reaction was concentrated, coevaporated once with DCM, and triturated 3× with ether to yield pure product.

### Intermediate 148-i: 3-(aminomethyl)-1-methylpyridin-1-ium

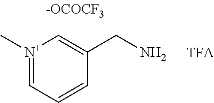

Prepared according to general method XXXVI to give 32 mg (11%) of the title compound as a white solid. ¹H NMR (400 MHz, Deuterium Oxide) δ 8.85 (s, 1H), 8.75 (d, J=6.0 Hz, 1H), 8.52 (d, J=8.2 Hz, 1H), 8.01 (t, J=7.0 Hz, 1H), 4.33 (d, J=4.3 Hz, 2H), 4.31 (s, 3H).

### Compound 148: 3-((6-bromo-5′-chloro-1H,1′H-[2,2′-biindole]-3-carboxamido)methyl)-1-methylpyridin-1-ium

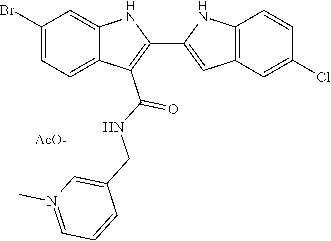 [SMILES: "AcO-" ; C[n+]1cccc(CNC(=O)c2c(-c3cc4cc(Cl)ccc4[nH]3)[nH]c3cc(Br)ccc23)c1]

Prepared according to general method XXXIV from intermediates 72-iii and 148-i and purified by directly loading into a flash purification 2-65% MeOH/CH₂Cl₂, then 65-95% (1.5% AcOH/MeOH)/CH₂Cl₂ to give the title compound as a white solid powder (23.1 mg, 54%). ¹H NMR (400 MHz, DMSO-d₆) δ 9.08 (s, 1H), 8.90 (d, J=6.0 Hz, 1H), 8.58 (d, J=8.1 Hz, 1H), 8.13 (t, J=7.0 Hz, 1H), 7.81 (d, J=8.5 Hz, 1H), 7.64 (d, J=1.8 Hz, 1H), 7.55 (d, J=2.0 Hz, 1H), 7.41 (d, J=8.6 Hz, 1H), 7.21 (dd, J=8.6, 1.8 Hz, 1H), 7.12 (s, 1H), 7.04 (d, J=8.6 Hz, 1H), 4.73 (s, 2H), 4.36 (s, 3H). Mass calculated for (C₂₄H₁₉BrClN₄O)⁺495.0, found 495.3.

### Intermediate 149-i: 4-(2-aminoethyl)-1-methylpyridin-1-ium

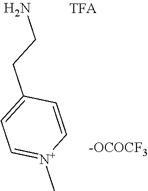

Prepared according to general method XXXVI to give the title compound as a white solid (405 mg, quant.). ¹H NMR (400 MHz, DMSO-d₆) δ 8.95 (d, J=6.2 Hz, 2H), 8.06 (d, J=6.2 Hz, 2H), 7.96 (s, 3H), 4.33 (s, 3H), 3.24 (s, 2H), 3.18 (d, J=6.8 Hz, 2H).

### Compound 149: 4-(2-(6-bromo-5′-chloro-1H,1′H-[2,2′-biindole]-3-carboxamido)ethyl)-1-methylpyridin-1-ium

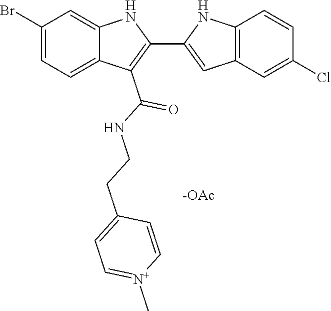 [SMILES: C[n+]1ccc(CCNC(=O)c2c(-c3cc4cc(Cl)ccc4[nH]3)[nH]c3cc(Br)ccc23)cc1]

Prepared according to general method XXXIV from 72-iii and 149-i and purified by directly loading into a flash purification 2-60% MeOH/CH₂Cl₂, then 60-95% (1.5% AcOH/MeOH)/CH₂Cl₂ to give the title compound as a white solid powder (15.0 mg, 34%). ¹H NMR (400 MHz, Methanol-d₄) δ 8.63 (d, J=6.2 Hz, 2H), 8.00 (d, J=6.2 Hz, 2H), 7.69-7.61 (m, 3H), 7.45 (d, J=8.6 Hz, 1H), 7.33 (dd, J=8.6, 1.8 Hz, 1H), 7.21 (dd, J=8.7, 2.1 Hz, 1H), 7.02 (s, 1H), 4.03 (s, 3H), 3.98 (t, J=6.6 Hz, 2H), 3.30 (t, J=6.7 Hz, 2H). Mass calculated for (C₂₅H₂₁BrClN₄O)⁺509.1, found 509.3.

### Intermediate 150-i: 2-(2-aminoethyl)-1-methylpyridin-1-ium

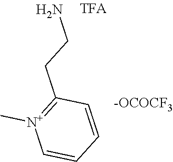

Prepared according to general method XXXVI to give the title compound as a white solid (430 mg, quant.). ¹H NMR (400 MHz, DMSO-d₆) δ 9.04 (d, J=6.1 Hz, 1H), 8.55 (t, J=7.9 Hz, 1H), 8.16 (s, 3H), 8.09-7.99 (m, 2H), 4.31 (s, 3H), 3.46-3.36 (m, 2H), 3.31 (p, J=6.8 Hz, 2H).

### Compound 150: 4-(2-(6-bromo-5′-chloro-1H,1′H-[2,2′-biindole]-3-carboxamido)ethyl)-1-methylpyridin-1-ium

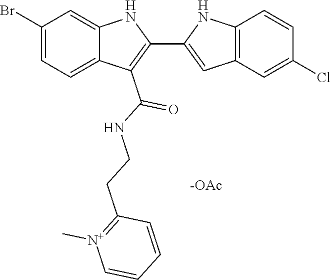 [SMILES: C[n+]1ccccc1CCNC(=O)c1c(-c2cc3cc(Cl)ccc3[nH]2)[nH]c2cc(Br)ccc12]

Prepared according to general method XXXIV from intermediates 72-iii and 150-i and purified direct loading into a flash purification 2-60% MeOH/CH₂Cl₂, then 60-95% (1.5% AcOH/MeOH)/CH₂Cl₂ to give the title compound as a white solid powder (15.0 mg, 34%). ¹H NMR (400 MHz, DMSO-d₆) δ 8.99 (d, J=6.1 Hz, 1H), 8.35 (t, J=7.8 Hz, 1H), 8.02 (d, J=8.0 Hz, 1H), 7.85 (t, J=6.9 Hz, 1H), 7.65-7.58 (m, 3H), 7.44 (d, J=8.7 Hz, 1H), 7.19 (d, J=8.7 Hz, 1H), 7.12-7.05 (m, 2H), 4.39 (s, 3H), 3.87 (t, J=6.7 Hz, 2H), 3.45 (t, J=6.5 Hz, 2H). Mass calculated for (C₂₅H₂₁BrClN₄O)⁺509.1, found 509.3.

### Intermediate 151-i: 2-amino-N-(methylsulfonyl)acetamide

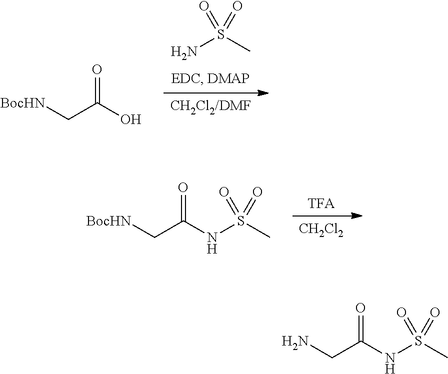

(tert-butoxycarbonyl)glycine (150 mg, 0.86 mmol), methylsulfonamide (123 mg, 1.29 mmol, 1.5 eq), EDC (198 mg, 1.03 mmol, 1.2 eq), and DMAP (158 mg, 1.29 mmol, 1.5 eq) were combined in 3 mL DCM/0.5 mL DMF. After 16 h, the reaction was concentrated, and the residues taken up in 20 mL EtOAc and extracted twice with 1M NaOH. The aqueous layer was acidified to pH ˜4 with 1 M citric acid. This was extracted 3× with EtOAc (25 mL), the combined organics washed with saturated brine, dried over anhydrous sodium sulfate, filtered, and concentrated to a clear oil.

The crude oil was treated with TFA (2 mL) in DCM (15 mL). After 30 min, the reaction was concentrated and coevaporated once with DCM. The crude was precipitated from DCM/MeOH with ether to form a sticky solid. This was further triturated with ether and sonication until the material was solid, and it was filtered to yield the title compound (210 mg, 83%). ¹H NMR (400 MHz, DMSO-d₆) δ 8.06 (s, 2H), 3.65 (s, 2H), 3.21 (s, 3H).

### Compound 151: 6-bromo-5′-chloro-N-(2-(methylsulfonamido)-2-oxoethyl)-1H,1′H-[2,2′-biindole]-3-carboxamide

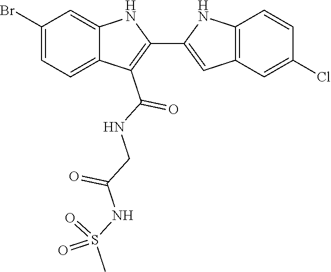 [SMILES: CS(=O)(=O)NC(=O)CNC(=O)c1c(-c2cc3cc(Cl)ccc3[nH]2)[nH]c2cc(Br)ccc12]

Prepared according to general method XXXIV from intermediates 72-iii and 151-i and purified by preparative HPLC to give the title compound as a white solid powder (18.4 mg, 41%). ¹H NMR (400 MHz, DMSO-d₆) δ 12.34 (s, 1H), 12.27 (s, 1H), 12.08 (s, 1H), 8.54 (t, J=5.8 Hz, 1H), 7.90 (d, J=8.6 Hz, 1H), 7.73 (d, J=2.1 Hz, 1H), 7.64 (d, J=1.8 Hz, 1H), 7.59 (d, J=8.7 Hz, 1H), 7.35 (dd, J=8.6, 1.9 Hz, 1H), 7.24-7.13 (m, 2H), 4.17 (d, J=5.8 Hz, 2H), 3.32 (s, 3H). ¹³C NMR (101 MHz, DMSO-d₆) δ 169.96, 166.39, 137.21, 135.21, 133.23, 131.22, 129.22, 125.59, 124.88, 124.13, 123.13, 122.70, 120.02, 116.15, 114.43, 114.04, 108.13, 102.08, 41.64. Mass calculated for (C₂₀H₁₆⁸¹BrClN₄O₄S+H)⁺525.0, found 525.3.

### General Method XXXVII

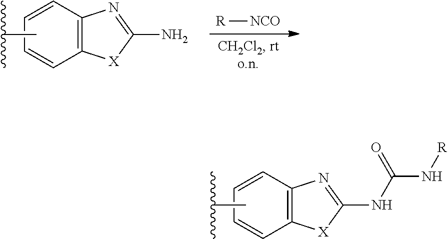

To a stirring solution or suspension of a benzothiazole-2-amine, or benzoxazole-2-amine in DCM (0.5-1 M), the required isocyanate (1.1 eq) was added, and the reaction stirred overnight. The white precipitate which formed was collected by filtration and washed with DCM to yield a product that was usually >95% pure.

### Compound 152: 1-(5-bromobenzo[d]thiazol-2-yl)-3-(4-methoxyphenyl)urea

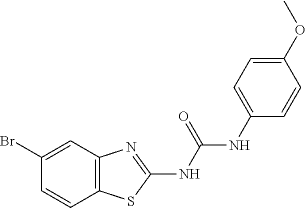 [SMILES: COc1ccc(NC(=O)Nc2nc3cc(Br)ccc3s2)cc1]

Prepared according to general method XXXVII from intermediate 99-i and 4-methoxyphenyl isocyanate to give the title compound as a white solid powder (26.3 mg, 16%). ¹H NMR (400 MHz, DMSO-d₆) δ 9.10 (s, 1H), 8.68 (s, 1H), 8.11 (d, J=2.3 Hz, 1H), 7.74 (d, J=8.7 Hz, 1H), 7.48 (dd, J=8.7, 2.4 Hz, 1H), 7.37 (d, J=9.0 Hz, 2H), 6.89 (d, J=9.0 Hz, 2H), 3.73 (s, 3H). ¹³C NMR (101 MHz, DMSO-d₆) δ 155.35, 152.75, 143.83, 134.93, 132.47, 126.50, 122.59, 121.00, 119.12, 116.13, 114.48, 111.63, 55.67. Mass calculated for (C₁₅H₁₂BrN₃O₂S+H) 378.0, found 378.3.

### Intermediate 153-i: 5-bromo-6-methoxybenzo[d]thiazol-2-amine (ALM00462-108)

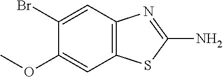

Prepared according to general method XXVII from 3-bromo-4-methoxyaniline to give the title compound as a light-yellow powder (1.5 g, 59%). ¹H NMR (400 MHz, DMSO-d₆) δ 7.52 (s, 1H), 7.51 (s, 1H), 7.42 (s, 2H), 3.82 (s, 3H). Mass calculated for (C₈H₇⁸¹BrN₂OS+H)⁺261.0, found 261.4.

### Compound 153: 1-(5-bromo-6-methoxybenzo[d]thiazol-2-yl)-3-phenylurea

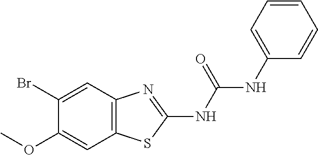 [SMILES: COc1cc2sc(NC(=O)Nc3ccccc3)nc2cc1Br]

Prepared according to general method XXXVII from 153-i and phenyl isocyanate to give the title compound as a white solid powder (26.3 mg, 16%). ¹H NMR (400 MHz, DMSO-d₆) δ 10.77 (s, 1H), 9.11 (s, 1H), 7.89 (s, 1H), 7.75 (s, 1H), 7.51 (d, J=8.0 Hz, 2H), 7.35 (d, J=7.7 Hz, 2H), 7.08 (t, J=7.4 Hz, 1H), 3.89 (s, 3H). Mass calculated for (C₁₅H₁₂⁸¹BrN₃O₂S+H)⁺380.0, found 380.3.

### Compound 154: 1-(5-bromobenzo[d]thiazol-2-yl)-3-(4-nitrophenyl)urea

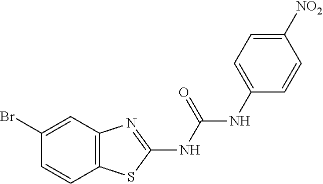 [SMILES: O=C(Nc1ccc([N+](=O)[O-])cc1)Nc1nc2cc(Br)ccc2s1]

Prepared according to general method XXXVII from 99-i and 4-nitrophenyl isocyanate to give the title compound as a yellow powder (435 mg, 84%). ¹H NMR (400 MHz, DMSO-d₆) δ 9.62 (s, 1H), 9.41 (s, 1H), 8.22 (d, J=9.1 Hz, 2H), 8.11 (d, J=2.3 Hz, 1H), 7.78 (d, J=8.6 Hz, 1H), 7.72 (d, J=9.2 Hz, 2H), 7.55 (dd, J=8.7, 2.4 Hz, 1H). ¹³C NMR (101 MHz, DMSO-d₆) δ 152.22, 146.23, 142.83, 141.91, 134.67, 126.16, 125.59, 123.18, 119.72, 118.40, 117.59, 111.51.

### Compound 155: 1-(5-bromobenzo[d]thiazol-2-yl)-3-(3,4-dichlorophenyl)urea

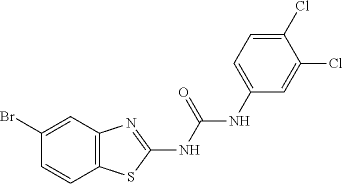 [SMILES: O=C(Nc1ccc(Cl)c(Cl)c1)Nc1nc2cc(Br)ccc2s1]

Prepared according to general method XXXVII from 99-i and 3,4-dichlorophenyl isocyanate to give the title compound as a white powder (100 mg, 55%). ¹H NMR (400 MHz, DMSO-d₆) δ 9.34 (s, 1H), 9.21 (s, 1H), 8.10 (d, J=2.4 Hz, 1H), 7.88 (d, J=2.5 Hz, 1H), 7.76 (d, J=8.7 Hz, 1H), 7.55 (d, J=8.8 Hz, 1H), 7.52 (dd, J=8.7, 2.4 Hz, 1H), 7.36 (dd, J=8.8, 2.5 Hz, 1H). ¹³C NMR (101 MHz, DMSO-d₆) δ 152.47, 143.13, 139.85, 134.76, 131.53, 131.10, 126.28, 124.21, 123.01, 120.20, 119.53, 119.24, 117.13, 111.58. Mass calculated for (C₁₄H₈⁷⁹BrCl₂N₃OS+H)⁺416.0, found 416.3.

### Compound 156: 1-(5-bromobenzo[d]thiazol-2-yl)-3-(4-chloro-3-(trifluoromethyl)phenyl)urea

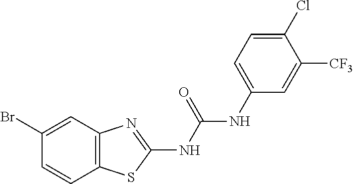 [SMILES: O=C(Nc1ccc(Cl)c(C(F)(F)F)c1)Nc1nc2cc(Br)ccc2s1]

Prepared according to general method XXXVII from 99-i and 3-trifluoromethyl-4-chlorophenyl isocyanate to give the title compound as a white powder (130 mg, 66%). ¹H NMR (400 MHz, DMSO-d₆) 9.37 (s, 2H), 8.10 (s, 1H), 8.10 (s, 1H), 7.75 (d, J=8.6 Hz, 1H), 7.67 (dd, J=8.9, 2.3 Hz, 1H), 7.63 (d, J=8.8 Hz, 1H), 7.54 (dd, J=8.7, 2.3 Hz, 1H). Mass calculated for (C₁₅H₈⁷⁹BrClF₃N₃OS+H)+450.0, found 450.4.

### Compound 157: 1-(5-bromobenzo[d]thiazol-2-yl)-3-(4-chloro-2-(trifluoromethyl)phenyl)urea

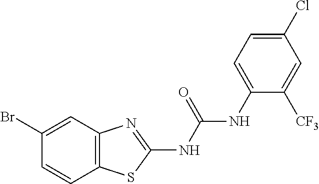 [SMILES: O=C(Nc1nc2cc(Br)ccc2s1)Nc1ccc(Cl)cc1C(F)(F)F]

Prepared according to general method XXXVII from 99-i and 2-trifluoromethyl-4-chlorophenyl isocyanate to give the title compound as a white powder (117 mg, 59%). ¹H NMR (400 MHz, DMSO-d₆) δ 9.83 (s, 1H), 8.34 (s, 1H), 8.13 (d, J=2.1 Hz, 1H), 7.96 (d, J=8.8 Hz, 1H), 7.77 (dq, J=11.1, 2.5 Hz, 3H), 7.47 (d, J=8.7 Hz, 1H). Mass calculated for (C₁₅H₈⁷⁹BrClF₃N₃OS+H)⁺ 450.0, found 450.4.

### Compound 158: 1-benzyl-3-(5-bromobenzo[d]thiazol-2-yl)urea

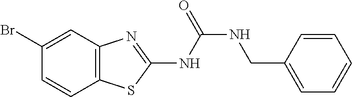 [SMILES: O=C(NCc1ccccc1)Nc1nc2cc(Br)ccc2s1]

Compound 99-i (100 mg, 0.437 mmol) was taken up in 1,2-DCE (1.5 mL) and benzyl isocyanate (0.054 mL, 0.524 mmol, 1.2 eq) was added, and the reaction heated to 60° C. for 22 h. The precipitate was filtered, washing with DCM to give the title compound as a white powder (39.7 mg, 25%). ¹H NMR (400 MHz, DMSO-d₆) δ 9.16 (s, 1H), 8.10 (s, 1H), 7.70 (d, J=8.4 Hz, 1H), 7.43 (dd, J=8.8, 2.2 Hz, 1H), 7.39-7.27 (m, 4H), 7.25 (t, J=7.0 Hz, 1H), 6.91 (t, J=6.1 Hz, 1H), 4.31 (d, J=5.9 Hz, 2H). ¹³C NMR (101 MHz, DMSO-d₆) δ 155.13, 144.40, 140.39, 135.06, 128.80, 127.61, 127.28, 126.66, 122.20, 118.68, 115.39, 111.73, 43.24. Mass calculated for (C₁₅H₁₂⁸¹BrN₃OS+H)⁺364.0, found 364.4.

### Compound 159: 1-(5-bromobenzo[d]thiazol-2-yl)-3-cyclohexylurea

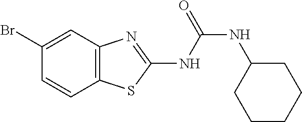 [SMILES: O=C(Nc1nc2cc(Br)ccc2s1)NC1CCCCC1]

Compound 99-i (100 mg, 0.437 mmol) was taken up in 1,2-DCE (1 mL) and cyclohexyl isocyanate (0.084 mL, 0.656 mmol, 1.5 eq) was added and the reaction was heated to 80° C. for 6 d. The reaction was placed in a 4° C. fridge for 3 h, precipitate filtered, and the filtrate was concentrated and purified by flash chromatography, then the still crude eluant was triturated with DCM to yield 14.6 mg (9%) of the title compound as a white solid. ¹H NMR (400 MHz, DMSO-d₆) δ 8.82 (s, 1H), 8.08 (d, J=2.3 Hz, 1H), 7.68 (d, J=8.7 Hz, 1H), 7.37 (dd, J=8.7, 2.3 Hz, 1H), 6.31 (d, J=7.8 Hz, 1H), 3.54-3.40 (m, 1H), 1.80 (d, J=13.2 Hz, 2H), 1.71-1.62 (m, 2H), 1.56-1.51 (m, 1H), 1.31 (q, J=12.2, 11.8 Hz, 2H), 1.18 (q, J=11.6, 10.7 Hz, 3H). ¹³C NMR (101 MHz, DMSO-d₆) δ 154.24, 144.47, 135.07, 126.70, 122.01, 118.51, 115.09, 111.70, 48.32, 33.18, 25.64, 24.80. Mass calculated for (C₁₄H₁₆⁸¹BrN₃OS+H)⁺356.0, found 356.4.

### Compound 160: 1-(4-aminophenyl)-3-(5-bromobenzo[d]thiazol-2-yl)urea

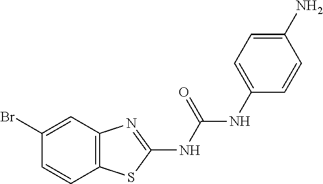 [SMILES: Nc1ccc(NC(=O)Nc2nc3cc(Br)ccc3s2)cc1]

Compound 154 (109 mg, 0.277 mmol) was taken up in EtOAc, purged with N₂, then 10% platinum on carbon (54 mg, 0.028 mmol, 0.1 eq) was added, and the reaction purged with H₂. After 3 h, the mixture was filtered through celite, washed with EtOAc and MeOH and concentrated to yield the title compound as a light-yellow solid (87 mg, 87%). ¹H NMR (400 MHz, DMSO-d₆) δ 9.04 (s, 1H), 8.36 (s, 1H), 8.11 (d, J=2.3 Hz, 1H), 7.72 (d, J=8.7 Hz, 1H), 7.46 (dd, J=8.7, 2.4 Hz, 1H), 7.08 (d, J=8.7 Hz, 2H), 6.52 (d, J=8.7 Hz, 2H), 4.84 (s, 2H). ¹³C NMR (101 MHz, DMSO-d₆) δ 152.86, 145.05, 144.14, 134.99, 128.18, 126.59, 122.40, 121.67, 118.92, 115.65, 114.50, 111.67.

### Compound 161: N-(4-(3-(5-bromobenzo[d]thiazol-2-yl)ureido)phenyl)-2-(dimethylamino)acetamide

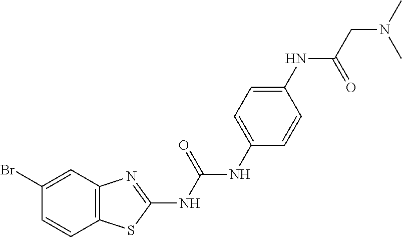 [SMILES: CN(C)CC(=O)Nc1ccc(NC(=O)Nc2nc3cc(Br)ccc3s2)cc1]

To a stirring solution of N,N-dimethylglycine (13.4 mg, 0.130 mmol, 1.3 eq) in 2 mL DCM/DMF (1:1) was added HATU (53 mg, 0.140 mmol, 1.4 eq) then DIPEA (0.061 mL, 0.35 mmol, 3.5 eq). After 3 min, compound 160 (36 mg, 0.100 mmol) was added in 1 mL DMF and the reaction stirred for 3 d. The mixture was diluted with 2 mL DCM and purified by flash chromatography (2-16% (5% NH₄OH/MeOH)/DCM), and the resultant oil was precipitated from MeOH/DCM with Et₂O to give a pinkish powder. Lyophilization of the powder from 1:1 MeOH/H₂O gave the title compound as a white solid (9.7 mg, 21%). ¹H NMR (400 MHz, DMSO-d₆) δ 10.41 (s, 1H), 9.73 (s, 1H), 9.17 (s, 1H), 8.88 (s, 1H), 8.12 (s, 1H), 7.75 (d, J=8.6 Hz, 1H), 7.56-7.49 (m, 2H), 7.49-7.41 (m, 2H), 4.04 (s, 2H), 2.84 (s, 6H). Mass calculated for (C₁₈H₁₈⁸¹BrN₅O₂S+H)⁺450.0, found 450.3.

### Compound 162: 1-(benzo[d]thiazol-2-yl)-3-(4-chlorophenyl)urea

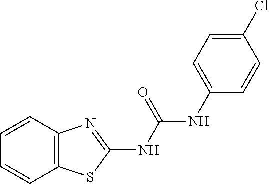 [SMILES: O=C(Nc1ccc(Cl)cc1)Nc1nc2ccccc2s1]

Prepared according to general method XXXVII from 2-aminobenzothiazole and 4-chlorophenyl isocyanate to give the title compound as a white powder (182 mg, 92%). ¹H NMR (400 MHz, DMSO-d₆) δ 11.01 (s, 1H), 9.34 (s, 1H), 7.90 (d, J=7.9 Hz, 1H), 7.65 (d, J=8.0 Hz, 1H), 7.58 (d, J=8.4 Hz, 2H), 7.44-7.35 (m, 3H), 7.25 (t, J=7.6 Hz, 1H). Mass calculated for (C₁₄H₁₀ClN₃OS+H)⁺304.0, found 304.4.

### General Method XXXVIII

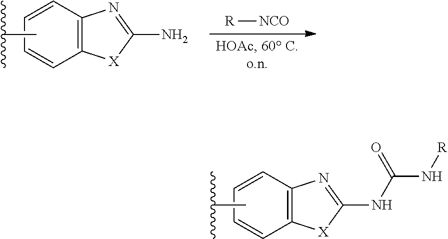

To a stirring solution or suspension of a benzothiazole-2-amine, or benzoxazole-2-amine in acetic acid (0.2-0.5 M), the required isocyanate (1.2 eq) was added, and the reaction stirred overnight at 60° C. The solution was concentrated and purified by flash chromatography and further precipitated, triturated, and/or recrystallized to obtain pure product.

### Compound 163: 1-(5-bromobenzo[d]thiazol-2-yl)-3-(pyridin-2-yl)urea

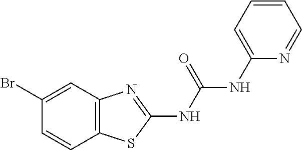 [SMILES: O=C(Nc1ccccn1)Nc1nc2cc(Br)ccc2s1]

Prepared according to general method XXXVIII from 99-i and 2-pyridyl isocyanate to give the title compound as a tan powder (15.2 mg, 10%). ¹H NMR (400 MHz, DMSO-d₆) δ 10.96 (s, 1H), 9.64 (s, 1H), 8.32 (d, J=4.9 Hz, 1H), 8.20 (d, J=2.3 Hz, 1H), 7.83-7.73 (m, 2H), 7.62 (dd, J=8.6, 2.3 Hz, 1H), 7.49 (d, J=8.4 Hz, 1H), 7.06 (t, J=6.2 Hz, 1H). ¹³C NMR (101 MHz, DMSO-d₆) δ 152.84, 152.45, 147.47, 142.74, 139.22, 134.68, 126.24, 123.36, 119.89, 118.45, 117.55, 112.54, 111.52. Mass calculated for (C₁₃H₉⁸¹BrN₄OS+H)⁺351.0, found 351.4.

### Compound 164: 1-(benzo[d]oxazol-2-yl)-3-(4-chlorophenyl)urea

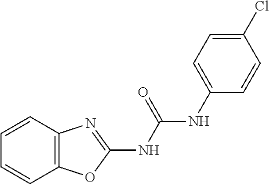 [SMILES: O=C(Nc1ccc(Cl)cc1)Nc1nc2ccccc2o1]

Prepared according to general method XXXVIII from 2-aminobenzoxazole and 4-chlorophenyl isocyanate to give the title compound as a white powder (155 mg, 72%). ¹H NMR (400 MHz, DMSO-d₆) δ 11.47 (s, 1H), 10.61 (s, 1H), 7.64 (s, 3H), 7.51-7.21 (m, 5H). Mass calculated for (C₁₄H₁₀ClN₃O₂+H)⁺288.0, found 288.5.

### Intermediate 165-i: 5-methoxybenzo[d]thiazol-2-amine

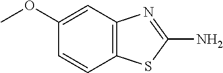

Prepared according to general method XXVII from 3-methoxyaniline to give the title compound (510 mg, 57%). ¹H NMR (400 MHz, DMSO-d₆) δ 7.22 (d, J=8.4 Hz, 1H), 6.33 (d, J=2.3 Hz, 1H), 6.23 (dd, J=8.5, 2.3 Hz, 1H), 5.84 (s, 2H), 3.82 (s, 3H). Mass calculated for (C₈H₈N₂OS+H)⁺181.0, found 181.4.

### Compound 165: 1-(4-chlorophenyl)-3-(5-methoxybenzo[d]thiazol-2-yl)urea

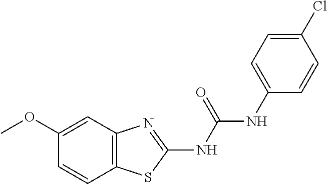 [SMILES: COc1ccc2sc(NC(=O)Nc3ccc(Cl)cc3)nc2c1]

Prepared according to general method XXXVII from intermediate 165-i and 4-chlorophenyl isocyanate to give the title compound as a white powder (84 mg, 73%). ¹H NMR (400 MHz, DMSO-d₆) δ 9.09 (s, 1H), 8.93 (s, 1H), 7.54-7.46 (m, 4H), 7.35 (d, J=8.8 Hz, 2H), 7.07 (dd, J=8.5, 2.1 Hz, 1H), 3.91 (s, 3H). Mass calculated for (C₁₅H₁₂ClN₃O₂S+H)⁺ 334.0, found 334.5.

### Intermediate 166-i: 5-fluorobenzo[d]thiazol-2-amine

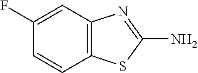 [SMILES: Nc1nc2cc(F)ccc2s1]

Prepared according to general method XXVII from 3-fluoroaniline to give the title compound (290 mg, 34%). ¹H NMR (400 MHz, DMSO-d₆) δ 7.35 (t, J=8.4 Hz, 1H), 6.56-6.39 (m, 2H), 6.18 (s, 2H). Mass calculated for (C₇H₅FN₂S+H) 169.0, found 169.4.

### Compound 166: 1-(4-chlorophenyl)-3-(5-fluorobenzo[d]thiazol-2-yl)urea

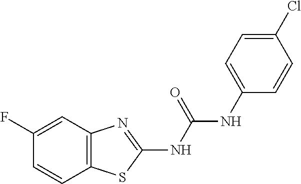 [SMILES: O=C(Nc1ccc(Cl)cc1)Nc1nc2cc(F)ccc2s1]

Prepared according to general method XXXVII from intermediate 166-i and 4-chlorophenyl isocyanate to give the title compound as a white powder (103.2 mg, 54%). ¹H NMR (400 MHz, DMSO-d₆) δ 9.31 (s, 1H), 9.05 (s, 1H), 7.77-7.64 (m, 2H), 7.50 (d, J=8.8 Hz, 2H), 7.36 (d, J=8.9 Hz, 2H), 7.29 (d, J=6.7 Hz, 1H). Mass calculated for (C₁₄H₉ClFN₃OS+H)⁺322.0, found 322.5.

### Intermediate 167-i: 5-nitrobenzo[d]thiazol-2-amine

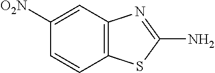 [SMILES: Nc1nc2cc([N+](=O)[O-])ccc2s1]

Prepared according to general method XXVII from 3-nitroaniline to give the title compound (150 mg, 15%). ¹H NMR (400 MHz, DMSO-d₆) δ 7.58 (d, J=8.6 Hz, 1H), 7.51 (d, J=2.7 Hz, 1H), 7.10 (dd, J=8.7, 2.7 Hz, 1H), 6.26 (s, 2H).

### Compound 167: 1-(4-chlorophenyl)-3-(5-nitrobenzo[d]thiazol-2-yl)urea

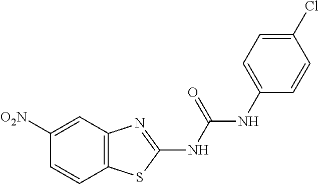 [SMILES: O=C(Nc1ccc(Cl)cc1)Nc1nc2cc([N+](=O)[O-])ccc2s1]

Prepared according to general method XXXVII from 167-i and 4-chlorophenyl isocyanate to give the title compound as an orange powder (55.0 mg, 31%). ¹H NMR (400 MHz, DMSO-d₆) δ 9.49 (s, 1H), 9.05 (s, 1H), 8.78 (s, 1H), 7.88 (s, 2H), 7.56-7.44 (m, 2H), 7.40-7.30 (m, 2H).

### Intermediate 168-i: 5-phenoxybenzo[d]thiazol-2-amine

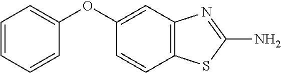 [SMILES: Nc1nc2cc(Oc3ccccc3)ccc2s1]

Prepared according to general method XXVII from 3-phenoxyaniline to give the title compound (70 mg, 6%). ¹H NMR (400 MHz, Chloroform-d) δ 7.49-7.32 (m, 3H), 7.21 (t, J=7.4 Hz, 1H), 7.09 (d, J=8.5 Hz, 2H), 6.44 (dd, J=8.5, 2.4 Hz, 1H), 6.16 (d, J=2.4 Hz, 1H). Mass calculated for (C₁₃H₁₀N₂OS+H)⁺243.0, found 243.5.

### Compound 168: 1-(4-chlorophenyl)-3-(5-phenoxybenzo[d]thiazol-2-yl)urea

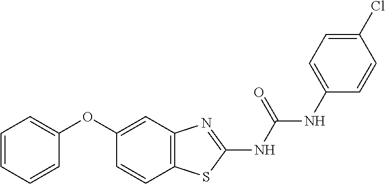 [SMILES: O=C(Nc1ccc(Cl)cc1)Nc1nc2cc(Oc3ccccc3)ccc2s1]

Prepared according to general method XXXVII from 168-i and 4-chlorophenyl isocyanate to give the title compound as a white powder (122.4 mg, 107%). ¹H NMR (400 MHz, DMSO-d₆) δ 9.12 (s, 1H), 8.82 (s, 1H), 7.66 (d, J=8.4 Hz, 1H), 7.48 (t, J=8.0 Hz, 2H), 7.47-7.38 (m, 2H), 7.37-7.20 (m, 5H), 7.12 (d, J=8.0 Hz, 2H). ¹³C NMR (101 MHz, DMSO-d₆) δ 157.01, 155.72, 152.46, 144.11, 138.58, 134.73, 130.83, 129.09, 126.26, 125.21, 120.51, 119.79, 114.67, 111.85, 108.09, 105.42. Mass calculated for (C₂₀H₁₄ClN₃O₂S+H)⁺396.0, found 396.4.

### Intermediate 169-i: 5-(trifluoromethyl)benzo[d]thiazol-2-amine

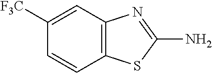 [SMILES: Nc1nc2cc(C(F)(F)F)ccc2s1]

Prepared according to general method XXVII from 3-trifluoromethylaniline to give the title compound (200 mg, 18%). ¹H NMR (400 MHz, DMSO-d₆) δ 7.62 (d, J=8.4 Hz, 1H), 7.08 (s, 1H), 6.85 (dd, J=8.6, 2.6 Hz, 1H), 6.37 (s, 2H). Mass calculated for (C₈H₅F₃N₂S+H)⁺219.0, found 219.4.

### Compound 169: 1-(4-chlorophenyl)-3-(5-(trifluoromethyl)benzo[d]thiazol-2-yl)urea

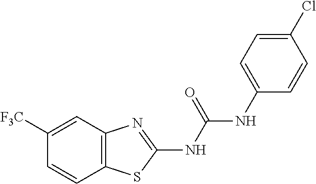 [SMILES: O=C(Nc1ccc(Cl)cc1)Nc1nc2cc(C(F)(F)F)ccc2s1]

Prepared according to general method XXXVII from 169-i and 4-chlorophenyl isocyanate to give the title compound as a white powder (108.5 mg, 64%). ¹H NMR (400 MHz, DMSO-d₆) δ 9.45 (s, 1H), 9.09 (s, 1H), 8.21 (s, 1H), 7.96 (d, J=8.5 Hz, 1H), 7.78 (d, J=8.3 Hz, 1H), 7.51 (d, J=8.8 Hz, 2H), 7.36 (d, J=6.7 Hz, 2H). Mass calculated for (C₁₅H₉ClF₃N₃OS+H)⁺ 372.0, found 372.4.

### Intermediate 170-i: 5-methylbenzo[d]thiazol-2-amine

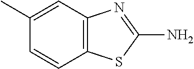

Prepared according to general method XXVII from 3-methylaniline to give the title compound (60 mg, 7%). ¹H NMR (400 MHz, Chloroform-d) δ 7.39 (d, J=8.4 Hz, 1H), 6.62 (d, J=2.7 Hz, 1H), 6.53 (dd, J=8.4, 2.7 Hz, 1H), 3.95 (s, 2H), 2.48 (s, 3H). Mass calculated for (C₈H₈N₂S+H)⁺ 165.0, found 165.5.

### Compound 170: 1-(4-chlorophenyl)-3-(5-methylbenzo[d]thiazol-2-yl)urea

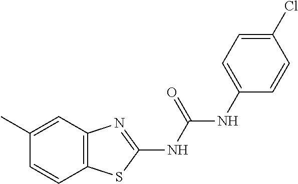 [SMILES: Cc1ccc2sc(NC(=O)Nc3ccc(Cl)cc3)nc2c1]

Prepared according to general method XXXVII from 170-i and 4-chlorophenyl isocyanate to give the title compound as a white powder (48.8 mg, 41%). ¹H NMR (400 MHz, DMSO-d₆) δ 8.99 (s, 1H), 8.93 (s, 1H), 7.61 (d, J=8.6 Hz, 1H), 7.55 (s, 1H), 7.49 (d, J=8.8 Hz, 2H), 7.45 (d, J=8.0 Hz, 1H), 7.34 (d, J=8.9 Hz, 2H), 2.47 (s, 3H). Mass calculated for (C₁₅H₁₂ClN₃OS+H)⁺ 318.0, found 318.5.

### Intermediate 171-i: 2-aminobenzo[d]thiazole-5-carboxylate

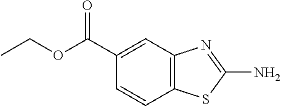

Prepared according to general method XXVII from ethyl 3-aminobenzoate to give the title compound as (412 mg, 37%). ¹H NMR (400 MHz, DMSO-d₆) δ 7.44 (d, J=8.7 Hz, 1H), 7.31 (d, J=2.7 Hz, 1H), 6.95 (dd, J=8.7, 2.7 Hz, 1H), 5.85 (s, 2H), 4.33 (q, J=7.1 Hz, 2H), 1.33 (t, J=7.1 Hz, 3H). Mass calculated for (C₁₀H₁₀N₂O₂S+H)⁺ 223.0, found 223.5.

### Compound 171: ethyl 2-(3-(4-chlorophenyl)ureido)benzo[d]thiazole-5-carboxylate

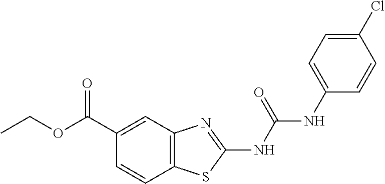 [SMILES: CCOC(=O)c1ccc2sc(NC(=O)Nc3ccc(Cl)cc3)nc2c1]

Prepared according to general method XXXVII from 171-i and 4-chlorophenyl isocyanate to give the title compound as an off-white powder (155 mg, 92%). ¹H NMR (400 MHz, DMSO-d₆) δ 9.25 (s, 1H), 8.91 (s, 1H), 8.40 (d, J=2.5 Hz, 1H), 7.81 (dd, J=8.9, 2.6 Hz, 1H), 7.74 (d, J=8.8 Hz, 1H), 7.51 (d, J=8.9 Hz, 2H), 7.35 (d, J=8.9 Hz, 1H), 4.39 (q, J=7.1 Hz, 2H), 1.36 (t, J=7.1 Hz, 3H). Mass calculated for (C₁₇H₁₄ClN₃O₃S+H)⁺ 376.0, found 376.5.

### Intermediate 172-i: 5-chlorobenzo[d]thiazol-2-amine

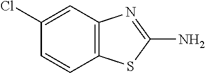 [SMILES: Nc1nc2cc(Cl)ccc2s1]

Prepared according to general method XXVII from 3-chloroaniline to give the title compound (420 mg, 46%). ¹H NMR (400 MHz, DMSO-d₆) δ 7.46 (dd, J=8.6, 2.0 Hz, 1H), 6.81 (t, J=2.3 Hz, 1H), 6.59 (dt, J=8.6, 2.4 Hz, 1H), 6.12 (s, 2H). Mass calculated for (C₇H₅ClN₂S+H)⁺185.0, found 185.4.

### Compound 172: 1-(5-chlorobenzo[d]thiazol-2-yl)-3-(4-chlorophenyl)urea

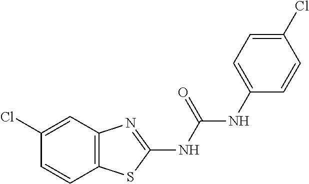 [SMILES: O=C(Nc1ccc(Cl)cc1)Nc1nc2cc(Cl)ccc2s1]

Prepared according to general method XXXVII from 172-i and 4-chlorophenyl isocyanate to give the title compound as a white powder (101 mg, 55%). ¹H NMR (400 MHz, DMSO-d₆) δ 9.24 (s, 1H), 9.03 (s, 1H), 7.96 (d, J=2.3 Hz, 1H), 7.75 (d, J=8.7 Hz, 1H), 7.50 (d, J=8.8 Hz, 2H), 7.46 (dd, J=8.7, 2.4 Hz, 1H), 7.35 (d, J=8.7 Hz, 2H). Mass calculated for (C₁₄H₉Cl₂N₃OS+H)⁺338.0, found 338.4.

### Compound 173: 1-(5-bromobenzo[d]thiazol-2-yl)-3-(tert-butyl)urea

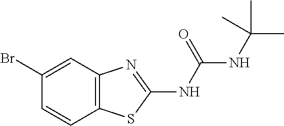 [SMILES: CC(C)(C)NC(=O)Nc1nc2cc(Br)ccc2s1]

Prepared according to general method XXXVIII from 99-i and tert-butyl isocyanate to give the title compound as a white powder (52.6 mg, 37%). ¹H NMR (400 MHz, DMSO-d₆) δ 8.76 (s, 1H), 8.08 (d, J=2.3 Hz, 1H), 7.67 (d, J=8.7 Hz, 1H), 7.30 (dd, J=8.7, 2.4 Hz, 1H), 6.19 (s, 1H), 1.29 (s, 9H). Mass calculated for (C₁₂H₁₄⁷⁹BrN₃OS+H)⁺ 328.0, found 328.4.

### Compound 174: 1-(5-bromobenzo[d]thiazol-2-yl)-3-cycloheptylurea

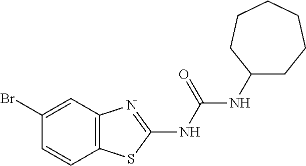 [SMILES: O=C(Nc1nc2cc(Br)ccc2s1)NC1CCCCCC1]

Prepared according to general method XXXVIII from 99-i and cycloheptyl isocyanate to give the title compound as a white powder (21.8 mg, 14%). ¹H NMR (400 MHz, DMSO-d₆) δ 8.81 (s, 1H), 8.09 (d, J=2.3 Hz, 1H), 7.68 (d, J=8.7 Hz, 1H), 7.36 (dd, J=8.7, 2.4 Hz, 1H), 6.34 (d, J=7.7 Hz, 1H), 3.74-3.61 (m, 1H), 1.88-1.79 (m, 2H), 1.64-1.39 (m, 10H). Mass calculated for (C₁₅H₁₈⁷⁹BrN₃OS+H)⁺368.0, found 368.4.

### Compound 175: N-(5-bromobenzo[d]thiazol-2-yl)acetamide

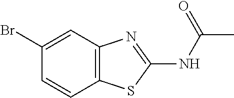

Prepared according to general method XXXVIII from 99-i and cyclohexyl isocyanate to give the title compound (a byproduct) as a white powder (47.1 mg, 17%). ¹H NMR (400 MHz, DMSO-d₆) δ 10.37 (s, 1H), 8.19 (d, J=2.3 Hz, 1H), 7.77 (d, J=8.7 Hz, 1H), 7.63 (dd, J=8.7, 2.3 Hz, 1H), 2.09 (s, 3H). Mass calculated for (C₉H₈⁷⁹BrN₂OS+H)⁺271.0, found 271.3.

### Compound 176: 1-(5-bromobenzo[d]thiazol-2-yl)-3-ethylurea

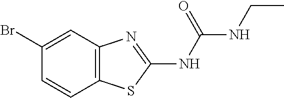 [SMILES: CCNC(=O)Nc1nc2cc(Br)ccc2s1]

Prepared according to general method XXXVIII from 99-i and ethyl isocyanate to give the title compound as a white powder (1.7 mg, 1.3%). NMR Mass calculated for (C₁₀H₁₀⁷⁹BrN₃OS+H)⁺ 300.0, found 300.3.

### General Method XXXIX

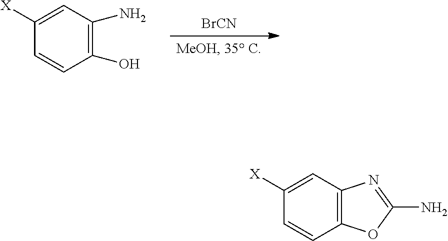

To a stirring solution of a 2-amino-4-halophenol in MeOH (0.5 M), cyanogen bromide (1.2 eq) was added portionwise. The mixture was heated to 35° C. for 2 h. To quench, sodium carbonate was added until the pH became neutral, then 100 mL EtOAc was added and the organic layer was washed 3× with water, once with saturated brine, dried over anhydrous sodium sulfate, filtered, and concentrated to yield a pure product.

### Intermediate 177-i: 5-chlorobenzo[d]oxazol-2-amine

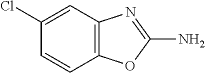 [SMILES: Nc1nc2cc(Cl)ccc2o1]

Prepared according to general method XXXIX from 2-amino-4-chlorophenol to yield the title compound as a brown solid (542 mg, 92%). ¹H NMR (400 MHz, DMSO-d₆) δ 7.60 (s, 2H), 7.33 (d, J=8.4 Hz, 1H), 7.23 (d, J=2.2 Hz, 1H), 6.98 (dd, J=8.4, 2.2 Hz, 1H). Mass calculated for (C₇H₅ClN₂O+H)⁺ 169.0, found 169.4.

### Compound 177: 1-(5-chlorobenzo[d]oxazol-2-yl)-3-(4-chlorophenyl)urea

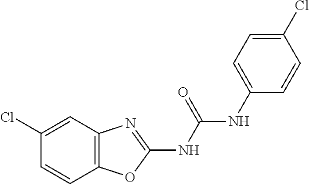 [SMILES: O=C(Nc1ccc(Cl)cc1)Nc1nc2cc(Cl)ccc2o1]

Prepared according to general method XXXVII from 177-i and 4-chlorophenyl isocyanate to give the title compound as a pink powder, (28 mg, 10%). ¹H NMR (400 MHz, DMSO-d₆) δ 11.55 (s, 1H), 10.44 (s, 1H), 7.80-7.58 (m, 4H), 7.47-7.10 (m, 3H). Mass calculated for (C₁₄H₉Cl₂N₃O₂+H)⁺322.0, found 322.5.

### Compound 178: 1-(5-chlorobenzo[d]oxazol-2-yl)-3-cyclohexylurea

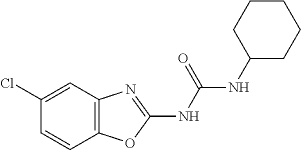 [SMILES: O=C(Nc1nc2cc(Cl)ccc2o1)NC1CCCCC1]

Prepared according to general method XXXVIII from 177-i and cyclohexyl isocyanate to give the title compound as a peach film (35.1 mg, 20%). ¹H NMR (400 MHz, Chloroform-d) δ 9.33 (s, 1H), 8.59 (d, J=7.9 Hz, 1H), 7.51 (d, J=2.1 Hz, 1H), 7.34 (d, J=8.6 Hz, 1H), 7.19 (dd, J=8.6, 2.2 Hz, 1H), 3.95-3.81 (m, 1H), 2.04 (d, J=11.3 Hz, 2H), 1.80 (dd, J=9.6, 4.4 Hz, 2H), 1.71-1.59 (m, 2H), 1.45 (q, J=11.6 Hz, 2H), 1.40-1.27 (m, 2H). Mass calculated for (C₁₄H₁₆ClN₃O₂+H)⁺ 294.0, found 294.6.

### Intermediate 179-i: 5-fluorobenzo[d]oxazol-2-amine

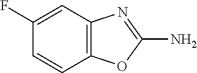 [SMILES: Nc1nc2cc(F)ccc2o1]

Prepared according to general method XXXIX from 2-amino-4-fluorophenol to yield the title compound as a brown solid (220 mg, 89%). ¹H NMR (400 MHz, DMSO-d₆) δ 7.54 (s, 2H), 7.30 (dd, J=8.7, 4.5 Hz, 1H), 7.02 (dd, J=9.4, 2.7 Hz, 1H), 6.75 (ddd, J=10.0, 8.6, 2.6 Hz, 1H). Mass calculated for (C₇H₅FN₂O+H)⁺153.0, found 153.4.

### Compound 179: 1-(4-chlorophenyl)-3-(5-fluorobenzo[d]oxazol-2-yl)urea

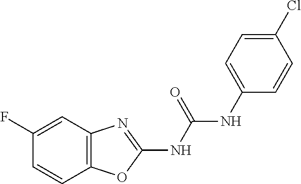 [SMILES: O=C(Nc1ccc(Cl)cc1)Nc1nc2cc(F)ccc2o1]

Prepared according to general method XXXVII from 179-i and 4-chlorophenyl isocyanate to give the title compound as a white powder (107 mg, 66%). ¹H NMR (400 MHz, DMSO-d₆) δ 11.52 (s, 1H), 10.45 (s, 1H), 7.79-7.53 (m, 3H), 7.53-7.20 (m, 3H), 7.09 (s, 1H). Mass calculated for (C₁₄H₉ClFN₃O₂+H)⁺306.0, found 306.5.

### Compound 180: 1-cyclohexyl-3-(5-fluorobenzo[d]oxazol-2-yl)urea

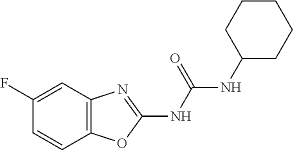 [SMILES: O=C(Nc1nc2cc(F)ccc2o1)NC1CCCCC1]

Prepared according to general method XXXVIII from 179-i and cyclohexyl isocyanate to give the title compound as an orange powder (45.3 mg, 17%). ¹H NMR (400 MHz, Chloroform-d) δ 9.43 (s, 1H), 8.62 (d, J=7.9 Hz, 1H), 7.34 (dd, J=8.8, 4.3 Hz, 1H), 7.21 (dd, J=8.5, 2.6 Hz, 1H), 6.93 (td, J=9.1, 2.6 Hz, 1H), 3.88 (s, 1H), 2.05 (d, J=6.9 Hz, 2H), 1.81 (dd, J=9.6, 4.3 Hz, 2H), 1.72-1.61 (m, 2H), 1.46 (q, J=11.4, 10.9 Hz, 2H), 1.50-1.24 (m, 2H). Mass calculated for (C₁₄H₁₈FN₃O₂+H)⁺278.1, found 278.6.

### Synthesis of Compound 181: Methyl 6-bromo-2-(3-(4-chlorophenyl)ureido)-1 H-indole-3-carboxylate

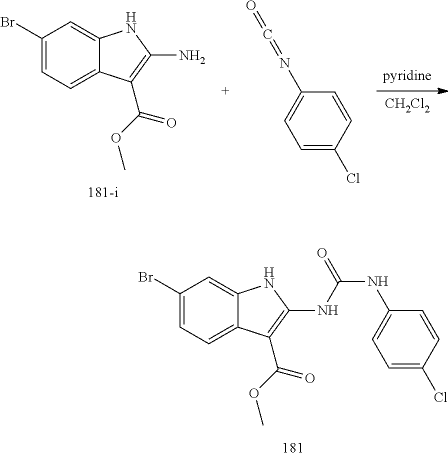

The 2-aminoindole intermediate 181-i was prepared according to literature procedures (WO 2011/056739). A mixture of methyl 2-amino-6-bromo-1H-indole-3-carboxylate 181-i (28 mg, 0.10 mmol), 4-chlorophenyl isocyanate (85 mg, 0.55 mmol), and pyridine (80 μL, 1.0 mmol) in 25% DMF/CH₂Cl₂ (1 mL) was stirred at ambient temperature for 12 days. The resulting suspension was filtered, the filtrate concentrated in vacuo and purified by column chromatography (eluted with 10-40% EtOAc/hexanes) to afford the desired urea 181 as a pink solid in 45% yield. ¹H NMR (400 MHz, DMSO-d₆) δ 12.14 (s, 1H), 10.52 (s, 1H), 10.02 (s, 1H), 7.73 (d, J=1.9 Hz, 1H), 7.67 (d, J=8.4 Hz, 1H), 7.61-7.53 (m, 2H), 7.46-7.38 (m, 2H), 7.26 (dd, J=8.4, 1.9 Hz, 1H), 3.89 (s, 3H). Mass calculated for (C₁₋₇H₁₃BrClN₃O₃−H)⁻ 420.0, found 420.3.

### Synthesis of Compound 182: Methyl 6-bromo-2-((4-chlorophenyl)sulfonamido)-1H-indole-3-carboxylate

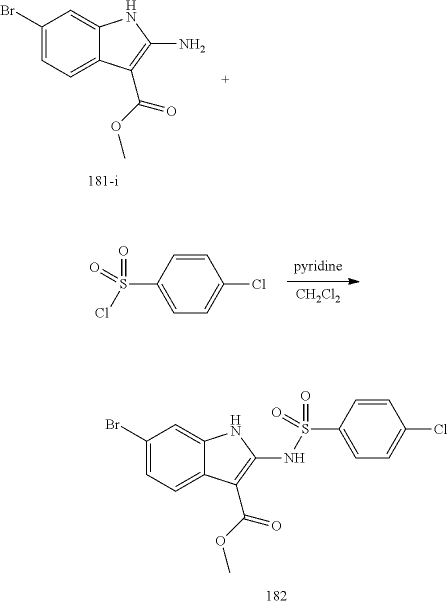

A mixture of methyl 2-amino-6-bromo-1H-indole-3-carboxylate 181-i (28 mg, 0.10 mmol), 4-chlorobenzenesulfonyl chloride (148 mg, 0.70 mmol), and pyridine (125 μL, 1.6 mmol) in CH₂Cl₂ (1 mL) was stirred at ambient temperature for 74 h. The reaction mixture was purified by column chromatography twice—first with 5-40% EtOAc/hexanes then with 0.2% MeOH/CH₂Cl₂—to afford the desired sulfonamide 182 as a white solid in 48% yield. ¹H NMR (400 MHz, DMSO-d₆) δ 12.12 (br s, 1H), 10.63 (br s, 1H), 7.79-7.70 (m, 3H), 7.70-7.60 (m, 3H), 7.28 (dd, J=8.5, 1.8 Hz, 1H), 3.56 (s, 3H). Mass calculated for (C₁₆H₁₂BrClN₂O₄S−H)-441.0, found 441.4.

### Compound 183: 3-(6-Bromo-5′-chloro-1H,1′H-[2,2′-biindole]-3-carboxamido)propyl Acetate

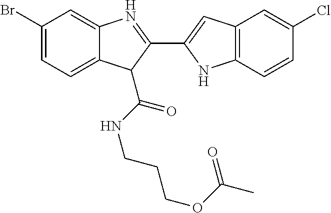

To a stirring suspension of glacial acetic acid (5.5 μL, 0.10 mmol) and HATU (44 mg, 0.12 mmol) in 30% DMF/CH₂Cl₂ (1 mL) was added DIPEA (49 μL, 0.28 mmol). After stirring for 10 min, alcohol 133 (24 mg, 0.054 mmol) was added and the resulting solution was stirring at 50° C. for 40 h. The mixture was diluted with EtOAc (5 mL) then washed with H₂O (2×) and brine (1×). The aqueous washes were combined and extracted with TBME (2×5 mL). The organics were combined, dried over MgSO₄, filtered, concentrated in vacuo and purified by column chromatography (eluted with 5-30% EtOAC/hexanes), followed by trituration (with CH₂Cl₂) to afford the desired acetyl ester 183 as light green solid in 74% yield. ¹H NMR (400 MHz, DMSO-d₆) δ 12.48 (s, 1H), 12.29 (s, 1H), 8.37 (s, 1H), 7.79-7.70 (m, 2H), 7.65-7.59 (m, 2H), 7.32 (dd, J=8.6, 1.8 Hz, 1H), 7.20-7.12 (m, 2H), 4.13 (t, J=6.4 Hz, 2H), 3.48 (q, J=6.5 Hz, 2H), 2.02 (s, 3H), 1.95 (q, J=6.6 Hz, 2H). Mass calculated for (C₂₂H₁₉BrClN₃O₃+H)⁺488.0, found 488.3.

### General Method XL

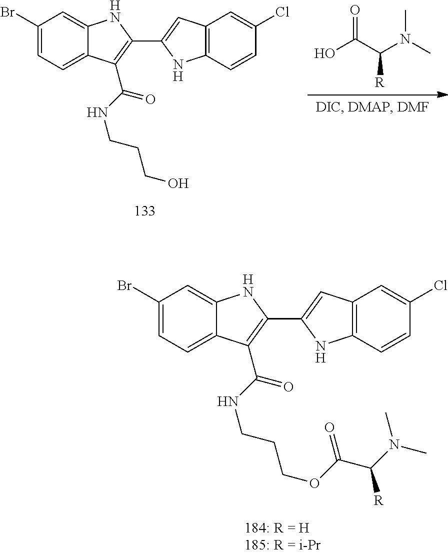

To a stirred suspension of alcohol 133 (1.0 eq), the appropriate acid (7.0 eq) and DMAP (8.0 eq) in DMF was added DIC (8.0 eq). The resulting mixture was stirred at 60° C. for 3 h, diluted with EtOAc, and washed with H₂O (2×) and brine (1×). The aqueous washes were combined and extracted with TBME (2×). The organics were combined, dried of MgSO₄, filtered, concentrated in vacuo and purified by column chromatography with gradients of EtOAc/hexanes. The residue was dissolved in minimal CH₂Cl₂ and precipitated with hexanes to afford the desired ester product.

### Compound 184: 3-(6-Bromo-5′-chloro-1H, 1′H-[2,2′-biindole]-3-carboxamido)propyl Dimethylglycinate

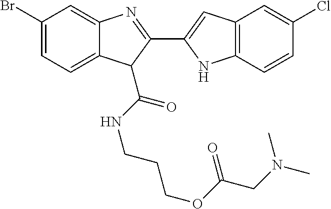 [SMILES: CN(C)CC(=O)OCCCNC(=O)C1C(c2cc3cc(Cl)ccc3[nH]2)=Nc2cc(Br)ccc21]

Prepared according to general method XL from N,N-dimethylglycine (15 mg, 42%). ¹H NMR (400 MHz, DMSO-d₆) 12.47 (s, 1H), 12.29 (s, 1H), 8.37 (t, J=5.7 Hz, 1H), 7.79-7.70 (m, 2H), 7.66-7.59 (m, 2H), 7.32 (dd, J=8.6, 1.9 Hz, 1H), 7.21-7.11 (m, 2H), 4.17 (t, J=6.3 Hz, 2H), 3.48 (q, J=6.4 Hz, 2H), 3.17 (s, 2H), 2.24 (s, 6H), 1.95 (p, J=7.5, 7.0 Hz, 2H). Mass calculated for (C₂₄H₂₄BrClN₄O₃+H)⁺531.1, found 531.4.

### Compound 185: 3-(6-Bromo-5′-chloro-1H,1′H-[2,2′-biindole]-3-carboxamido)propyl Dimethyl-L-valinate

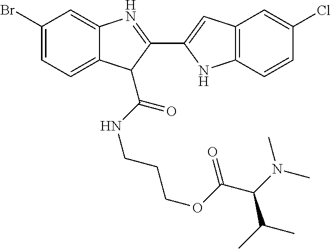

Prepared according to general method XL from N,N-dimethyl-L-valine (15 mg, 42%). ¹H NMR (400 MHz, DMSO-d₆) b 12.48 (s, 1H), 12.29 (s, 1H), 8.37 (t, J=5.6 Hz, 1H), 7.79-7.70 (m, 2H), 7.65-7.56 (m, 2H), 7.32 (dd, J=8.6, 1.8 Hz, 1H), 7.21-7.12 (m, 2H), 4.20 (td, J=6.4, 2.3 Hz, 2H), 3.49 (q, J=6.6 Hz, 2H), 2.68 (d, J=10.5 Hz, 1H), 2.22 (s, 6H), 2.03-1.84 (m, 3H), 0.90 (d, J=6.6 Hz, 3H), 0.82 (d, J=6.6 Hz, 3H). Mass calculated for (C₂₇H₃₀BrClN₄O₃+H)⁺573.1, found 573.4.

### Synthesis of Compounds 186-194

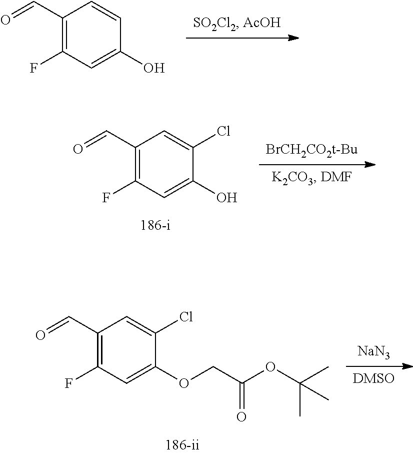

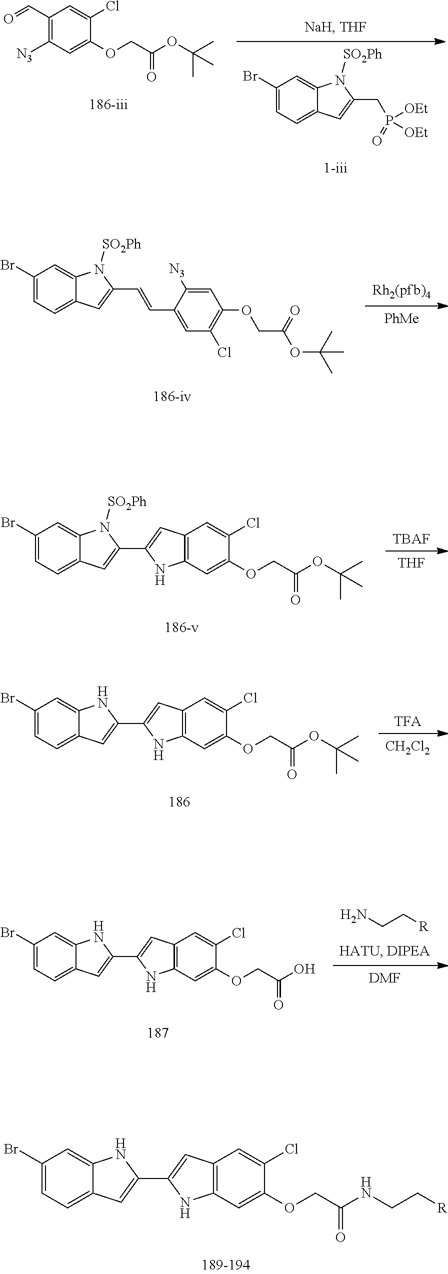

### Intermediate 186-i: 5-Chloro-2-fluoro-4-hydroxybenzaldehyde

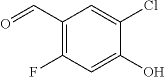 [SMILES: O=Cc1cc(Cl)c(O)cc1F]

Sulfuryl chloride (3.3 mL, 40.7 mmol) was added to a stirring suspension of 2-fluoro-4-hydroxybenzaldehyde (3.78 g, 27.0 mmol) in glacial acetic acid (27 mL). The resulting mixture was stirred at ambient temperature for 21 h and then quenched with H₂O (200 mL). The mixture was extracted with EtOAc (3×100 mL). The organic extracts were combined, washed with H₂O (2×) and brine (1×), dried over MgSO₄, filtered, concentrated in vacuo and purified by column chromatography (eluted with 5-50% Et₂O/hexanes) to afford the desired chloride 186-i as a white solid in 43% yield. ¹H NMR (400 MHz, DMSO-d₆) δ 12.06 (s, 1H), 9.98 (s, 1H), 7.81 (d, J=7.3 Hz, 1H), 6.88 (d, J=12.0 Hz, 1H). Mass calculated for (C₇H₄ClFO₂−H)⁻ 173.0, found 173.5.

### Intermediate 186-ii: Tert-Butyl 2-(2-chloro-5-fluoro-4-formylphenoxy)acetate

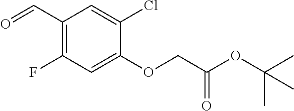 [SMILES: CC(C)(C)OC(=O)COc1cc(F)c(C=O)cc1Cl]

tert-Butyl bromoacetate (1.13 mL, 7.7 mmol) was added to a stirring suspension of chloride 186-i (1.16 g, 6.6 mmol) and K₂CO₃ (1.13 g, 8.2 mmol) in DMF (6 mL). The resulting mixture was stirred at ambient temperature for 18 h and quenched with H₂O (30 mL). The mixture was extracted with Et₂O (40 mL). The organic extract was washed with H₂O (3×40 mL) and brine (20 mL). The aqueous washes were combined and extracted with more Et₂O (2×25 mL). The combined organics was dried over MgSO₄, filtered, concentrated in vacuo and purified by column chromatography (eluted with 5-30% Et₂O/hexanes) to afford the desired phenolic ester 186-ii as a white solid in 92% yield. ¹H NMR (400 MHz, CDCl₃) δ 10.21 (s, 1H), 7.94 (d, J=7.2 Hz, 1H), 6.59 (d, J=11.4 Hz, 1H), 4.69 (s, 2H), 1.53 (s, 9H). λₘₐₓ=264.5 nm

### Intermediate 186-iii: Tert-Butyl 2-(5-azido-2-chloro-4-formylphenoxy)acetate

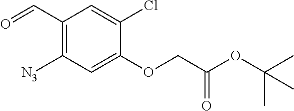 [SMILES: CC(C)(C)OC(=O)COc1cc(N=[N+]=[N-])c(C=O)cc1Cl]

A suspension of phenoxide 186-ii (1.75 g, 6.1 mmol) and sodium azide (0.80 g, 12.4 mmol) in DMSO (15 mL) was stirring at 50° C. for 42 h and then partitioned between TBME (120 mL) and H₂O (60 mL). The organic layer was successively washed with sat. aq. NH₄Cl (50 mL), H₂O (50 mL) and brine (50 mL). The aqueous washes were combined and then extracted with TBME (2×35 mL). The organic extracts were combined, dried over MgSO₄, filtered and concentrated in vacuo. The pale yellow crude solid was triturated with Et₂O, collected via filtration, rinsed with more Et₂O to afford the desired aryl azide 186-iii as an off-white solid in 95% yield. ¹H NMR (400 MHz, CDCl₃) δ 10.19 (s, 1H), 7.95 (s, 1H), 6.57 (s, 1H), 4.73 (s, 2H), 1.54 (s, 9H). Mass calculated for (C₁₃H₁₄ClN₃O₄−N₂+H)⁺284.1, found 284.3.

### Intermediate 186-iv: tert-Butyl (E)-2-(5-azido-4-(2-(6-bromo-1-(phenylsulfonyl)-1H-indol-2-yl)vinyl)-2-chlorophenoxy)acetate

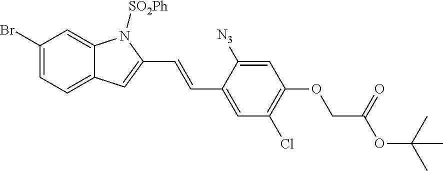

Sodium hydride (60% dispersion, 0.13 g, 3.2 mmol) was added to a cold (0° C.) stirring solution of phosphonate 1-iii (1.22 g, 2.5 mmol) in THF (30 mL) under N₂. The resulting mixture was stirred for 15 min and then azide 186-iii (0.86 g, 2.8 mmol) was added in one portion. The resulting brown/brick red mixture was stirred cold for 5 min then at ambient temperature for 90 min. The mixture was concentrated in vacuo, co-evaporated with CH₂Cl₂ (3×) to afford a very viscous deep red oil/paste which was sonicated with MeOH (40 mL) for several minutes until a uniform light orange suspension was obtained. This mixture was stirred for an additional 90 min and then the yellow solid was collected by filtration, rinsed with MeOH, dried in vacuo to afford the desired vinyl azide 186-iv in 62% yield. ¹H NMR (400 MHz, CDCl₃) δ 8.43 (s, 1H), 7.81-7.71 (m, 3H), 7.67 (d, J=16.2 Hz, 1H), 7.56 (t, J=7.5 Hz, 1H), 7.47-7.31 (m, 4H), 7.14 (d, J=16.3 Hz, 1H), 6.82 (s, 1H), 6.59 (s, 1H), 4.69 (s, 2H), 1.56 (s, 9H). Mass calculated for (C₂₈H₂₄BrClN₄O₅S−N₂+Na)⁺637.0, found 637.3.

### Intermediate 186-v: tert-Butyl 2-((6′-bromo-5-chloro-1′-(phenylsulfonyl)-1H,1′H-[2,2′-biindol]-6-yl)oxy)acetate

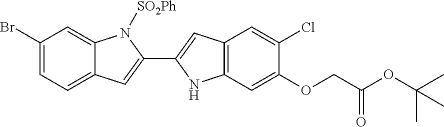

A mixture of vinyl azide 186-iv (1.23 g, 1.9 mmol), rhodium(II) perfluorobutyrate dimer (59 mg, 0.055 mmol, 3.0 mol %) and toluene (20 mL) was stirred at 100° C. for 3 h. The mixture was concentrated in vacuo, co-evaporated with Et₂O to afford a light brown solid crude 186-v which was carried onto the next step without further purification. ¹H NMR (400 MHz, CDCl₃) δ 8.93 (s, 1H), 8.56 (s, 1H), 7.65 (s, 1H), 7.56-7.32 (m, 7H), 6.98 (s, 1H), 6.75 (s, 1H), 6.57-6.50 (m, 1H), 4.69 (s, 2H), 1.55 (s, 9H). Mass calculated for (C₂₈H₂₄BrClN₂O₅S+H)⁺615.0, found 615.3.

### Compound 186: tert-Butyl 2-((6′-bromo-5-chloro-1H,1′H-[2,2′-biindol]-6-yl)oxy)acetate

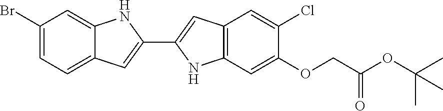 [SMILES: CC(C)(C)OC(=O)COc1cc2[nH]c(-c3cc4ccc(Br)cc4[nH]3)cc2cc1Cl]

Crude protected biindole 186-v was dissolved in THF (40 mL) and treated with a 1M solution of TBAF in THF (10 mL). The mixture was stirring at 60° C. for 2 h and the concentrated in vacuo. The residue was dissolved in EtOAc (60 mL), washed with H₂O (3×40 mL) and brine (50 mL), dried over MgSO₄, filtered, concentrated in vacuo and purified by column chromatography (eluted with 25% EtOAc/hexanes) to afford the desired biindole ester 186 in quantitative yield over two steps. ¹H NMR (400 MHz, DMSO-d₆) δ 11.70 (s, 1H), 11.62 (s, 1H), 7.67 (s, 1H), 7.54 (s, 1H), 7.52 (s, 1H), 7.14 (dd, J=8.3, 1.8 Hz, 1H), 6.92 (s, 1H), 6.91-6.83 (m, 2H), 4.80 (s, 2H), 1.47 (s, 9H). Mass calculated for (C₂₂H₂₀BrClN₂O₃+H)⁺475.0, found 475.3.

### Compound 187: 2-((6′-Bromo-5-chloro-1H,1′H-[2,2′-biindol]-6-yl)oxy)acetic Acid

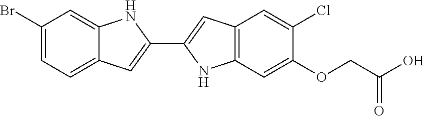 [SMILES: O=C(O)COc1cc2[nH]c(-c3cc4ccc(Br)cc4[nH]3)cc2cc1Cl]

TFA (21 mL) was added to a stirring suspension of biindole ester 186 (0.91 g, 1.9 mmol) in CH₂Cl₂ (85 mL). The resulting red solution was stirred at ambient temperature for 2 h. The mixture was then concentrated in vacuo, co-evaporated with MeOH and CH₂Cl₂ and sonicated with a minimal amount of 5% MeOH/CH₂Cl₂. The resulting maroon solid was collected, rinsed with CH₂Cl₂ to afford the desired biinolde acid 187 in 46% yield. ¹H NMR (400 MHz, DMSO-d₆) δ 13.15 (br s, 1H), 11.70 (d, J=2.1 Hz, 1H), 11.63 (d, J=2.2 Hz, 1H), 7.67 (s, 1H), 7.53 (d, J=2.0 Hz, 1H), 7.52 (s, 1H), 7.14 (dd, J=8.4, 1.8 Hz, 1H), 6.94 (s, 1H), 6.87 (dd, J=8.9, 2.0 Hz, 2H), 4.82 (s, 2H). Mass calculated for (C₁₈H₁₂BrClN₂O₃+H)⁺419.0, found 419.3.

### Compound 188: Methyl 2-((6′-bromo-5-chloro-1H,1′H-[2,2′-biindol]-6-yl)oxy)acetate

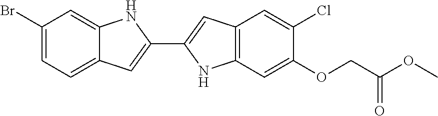 [SMILES: COC(=O)COc1cc2[nH]c(-c3cc4ccc(Br)cc4[nH]3)cc2cc1Cl]

The filtrate from the preparation of biinolde acid 187 was concentrated in vacuo and the residue was dissolved in MeOH. After standing for several days, a solid was collected. Analysis by ¹H NMR spectroscopy shows a 4:1 mixture of methyl ester to acid. The mixture was purified via preparative HPLC (eluted with 60-80% MeCN/H₂O, with 0.1% formic acid) to afford the desired ester 188 as a pink solid in 69% yield. ¹H NMR (400 MHz, DMSO-d₆) δ 11.73 (d, J=2.1 Hz, 1H), 11.67 (d, J=2.1 Hz, 1H), 7.68 (s, 1H), 7.56-7.50 (m, 2H), 7.14 (dd, J=8.4, 1.8 Hz, 1H), 6.95 (s, 1H), 6.87 (dd, J=12.6, 1.9 Hz, 2H), 4.96 (s, 2H), 3.74 (s, 3H). Mass calculated for (C₁₉H₁₄BrClN₂O₃+H)⁺433.0, found 433.3.

### General Method XLI

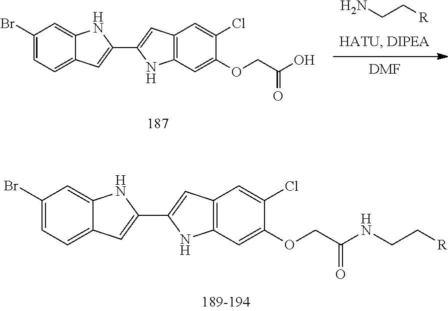

To a stirred solution of biindole acid 185 (1.0 eq) in DMF was added HATU (1.3 eq), then DIPEA (4-6 eq). After 3-5 min, the appropriate amine (1.3 eq) was added. The mixture was stirred for 16-24 h. In some cases, the resulting precipitate was collected and rinsed with Et₂O. In other cases, the reaction mixture was directly loaded onto a silica gel column and eluted with gradients of either (5% AcOH/MeOH)/CH₂Cl₂ or (2% formic acid/MeOH)/CH₂Cl₂ to yield pure product.

### Compound 189: 2-((6′-Bromo-5-chloro-1H,1′H-[2,2′-biindol]-6-yl)oxy)-N-(2-(dimethylamino)ethyl)acetamide

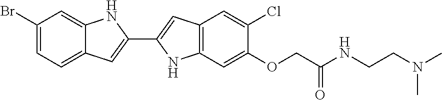 [SMILES: CN(C)CCNC(=O)COc1cc2[nH]c(-c3cc4ccc(Br)cc4[nH]3)cc2cc1Cl]

Prepared according to general method XLI from N,N-dimethylethylenediamine (58 mg, 80%). ¹H NMR (400 MHz, DMSO-d₆) δ 11.73 (d, J=2.0 Hz, 1H), 11.70 (s, 1H), 7.87 (t, J=5.5 Hz, 1H), 7.69 (s, 1H), 7.54 (d, J=2.0 Hz, 1H), 7.52 (s, 1H), 7.14 (dd, J=8.4, 1.7 Hz, 1H), 7.01 (s, 1H), 6.93-6.83 (m, 2H), 4.62 (s, 2H), 3.27 (q, J=6.2 Hz, 2H), 2.36 (t, J=6.6 Hz, 2H), 2.17 (s, 6H). Mass calculated for (C₂₂H₂₂BrClN₄O₂+H)⁺489.1, found 489.4.

### Compound 190: 1-(3-(2-((6′-Bromo-5-chloro-1H,1′H-[2,2′-biindol]-6-yl)oxy)acetamido)propyl)pyridin-1-ium Acetate

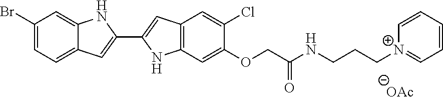 [SMILES: O=C(COc1cc2[nH]c(-c3cc4ccc(Br)cc4[nH]3)cc2cc1Cl)NCCC[n+]1ccccc1 ; "OAc"]

Prepared according to general method XLI from 1-(2-aminoethyl)pyridin-1-ium bromide 144-i (23 mg, 58%). ¹H NMR (400 MHz, DMSO-d₆) δ 13.57 (br s, 2H), 9.10 (d, J=6.0 Hz, 2H), 8.58 (t, J=7.8 Hz, 1H), 8.39 (s, 1H), 8.13 (t, J=7.0 Hz, 2H), 7.62 (d, J=1.3 Hz, 1H), 7.54 (d, J=1.8 Hz, 1H), 7.47 (d, J=8.5 Hz, 1H), 7.14-7.05 (m, 2H), 6.85 (d, J=10.9 Hz, 2H), 4.63 (d, J=9.4 Hz, 4H), 3.25 (d, J=6.3 Hz, 2H), 2.18 (t, J=6.9 Hz, 2H), 1.70 (d, J=2.7 Hz, 5H). Mass calculated for (C₂₆H₂₃BrClN₄O₂)⁺537.1, found 537.4.

### Compound 191: 3-(2-((6′-Bromo-5-chloro-1H,1′H-[2,2′-biindol]-6-yl)oxy)acetamido)-N,N,N-trimethylpropan-1-aminium Formate

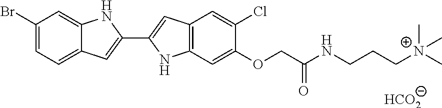

Prepared according to general method XLI from 3-amino-N,N,N-trimethylpropan-1-aminium 147-i (33 mg, 80%). ¹H NMR (400 MHz, DMSO-d₆) δ 12.63 (s, 2H), 8.56 (br s, 2H), 8.30 (s, 1H), 7.66 (s, 1H), 7.56 (d, J=1.7 Hz, 1H), 7.50 (d, J=8.4 Hz, 1H), 7.12 (dd, J=8.4, 1.8 Hz, 1H), 7.06 (s, 1H), 6.89 (dd, J=10.9, 1.9 Hz, 2H), 4.63 (s, 2H), 3.27 (t, J=7.9 Hz, 4H), 3.01 (s, 9H), 1.91 (s, 2H). Mass calculated for (C₂₄H₂₇BrClN₄O₂)⁺ 517.1, found 517.4.

### Compound 192: 2-((6′-Bromo-5-chloro-1H,1′H-[2,2′-biindol]-6-yl)oxy)-N-(2-hydroxyethyl)acetamide

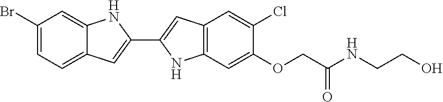 [SMILES: O=C(COc1cc2[nH]c(-c3cc4ccc(Br)cc4[nH]3)cc2cc1Cl)NCCO]

Prepared according to general method XLI from 2-aminoethanol (22 mg, 68%). ¹H NMR (400 MHz, DMSO-d₆) δ 11.72 (d, J=2.1 Hz, 1H), 11.68 (d, J=2.1 Hz, 1H), 7.91 (t, J=5.8 Hz, 1H), 7.68 (s, 1H), 7.57-7.50 (m, 2H), 7.14 (dd, J=8.3, 1.8 Hz, 1H), 7.02 (s, 1H), 6.93-6.82 (m, 2H), 4.79 (t, J=5.3 Hz, 1H), 4.62 (s, 2H), 3.48 (q, J=5.8 Hz, 2H), 3.27 (q, J=6.0 Hz, 2H). Mass calculated for (C₂₀H₁₇BrClN₃O₃+H)⁺462.0, found 462.2.

### Compound 193: 2-((6′-Bromo-5-chloro-1H,1′H-[2,2′-biindol]-6-yl)oxy)-N-(2-(tert-butylamino)ethyl)acetamide

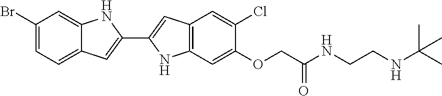 [SMILES: CC(C)(C)NCCNC(=O)COc1cc2[nH]c(-c3cc4ccc(Br)cc4[nH]3)cc2cc1Cl]

Prepared according to general method XLI from N¹-(tert-butyl)ethane-1,2-diamine (23 mg, 50%). ¹H NMR (400 MHz, DMSO-d₆) δ 11.69 (d, J=10.3 Hz, 2H), 7.87 (t, J=5.4 Hz, 1H), 7.69 (s, 1H), 7.56-7.50 (m, 2H), 7.14 (dd, J=8.4, 1.8 Hz, 1H), 7.02 (s, 1H), 6.92-6.83 (m, 2H), 4.62 (s, 2H), 3.21 (q, J=6.0 Hz, 2H), 2.58 (d, J=6.5 Hz, 2H), 1.39 (s, 1H), 0.99 (s, 9H). Mass calculated for (C₂₄H₂₆BrClN₄O₂+H)⁺517.1, found 517.0.

### Compound 194: 2-((6′-Bromo-5-chloro-1H,1′H-[2,2′-biindol]-6-yl)oxy)-N-(2-(4-methyl-2-phenylpiperazin-1-yl)ethyl)acetamide

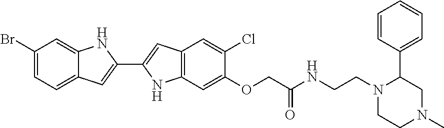 [SMILES: CN1CCN(CCNC(=O)COc2cc3[nH]c(-c4cc5ccc(Br)cc5[nH]4)cc3cc2Cl)C(c2ccccc2)C1]

Prepared according to general method XLI from 2-(4-methyl-2-phenylpiperazin-1-yl)ethan-1-amine (25 mg, 45%). ¹H NMR (400 MHz, DMSO-d₆) δ 11.83-11.60 (m, 2H), 7.73 (s, 1H), 7.63 (d, J=6.2 Hz, 1H), 7.57-7.50 (m, 2H), 7.35-7.28 (m, 2H), 7.26-7.17 (m, 3H), 7.14 (dd, J=8.5, 1.7 Hz, 1H), 7.04 (s, 1H), 6.95-6.84 (m, 2H), 4.60 (d, J=2.3 Hz, 2H), 3.29-3.22 (m, 2H), 3.16 (d, J=11.4 Hz, 1H), 3.09 (d, J=11.5 Hz, 1H), 2.75 (d, J=10.6 Hz, 1H), 2.60 (d, J=11.1 Hz, 2H), 2.23 (t, J=11.1 Hz, 1H), 2.14 (s, 4H), 1.98 (d, J=12.2 Hz, 1H), 1.86 (s, 1H). Mass calculated for (C₃₁H₃₁BrClN₅O₂+H)⁺620.1, found 620.0.

### Compound 195: N-(4-(N-(2-((6′-Bromo-5-chloro-1H, 1′H-[2,2′-biindol]-6-yl)oxy)acetyl)sulfamoyl)phenyl)-2,2,2-trifluoroacetamide

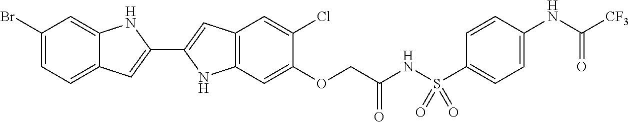 [SMILES: O=C(COc1cc2[nH]c(-c3cc4ccc(Br)cc4[nH]3)cc2cc1Cl)NS(=O)(=O)c1ccc(NC(=O)C(F)(F)F)cc1]

A mixture of biindole acid 187 (63 mg, 0.15 mmol), 2,2,2-trifluoro-N-(4-sulfamoylphenyl)acetamide (93 mg, 0.35 mmol), DCC (71 mg, 0.35 mmol) and DMAP (42 mg, 0.35 mmol) in 15% DMF/CH₂Cl₂ (5 mL) was stirred at ambient temperature for 112 h. The resulting suspension was filtered and the collected solid was purified via preparative HPLC (eluted with 60-80% MeCN/H₂O, with 0.1% formic acid) to afford the desired N-acyl sulfonamide 195 as a brown solid in 22% yield. ¹H NMR (400 MHz, DMSO-d₆) δ 12.63 (s, 1H), 11.83-11.59 (m, 3H), 8.02 (dd, J=8.9, 2.3 Hz, 2H), 7.94 (dd, J=8.9, 2.3 Hz, 2H), 7.66 (d, J=2.1 Hz, 1H), 7.57-7.46 (m, 2H), 7.15 (dt, J=8.4, 2.0 Hz, 1H), 6.94-6.78 (m, 3H), 4.85-4.73 (m, 2H). Mass calculated for (C₂₆H₁₇BrClF₃N₄O₃S+H)⁺669.0, found 669.2.

### Compound 196: 2-((6′-Bromo-5-chloro-1H,1′H-[2,2′-biindol]-6-yl)oxy)ethan-1-ol

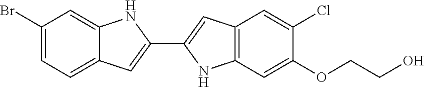 [SMILES: OCCOc1cc2[nH]c(-c3cc4ccc(Br)cc4[nH]3)cc2cc1Cl]

Lithium aluminum hydride (32 mg, 0.84 mmol) was added portion-wise (4×8 mg, 0.5 h intervals) to a stirring solution of biindole t-butyl ester 186 (49 mg, 0.10 mmol) in THF (3 mL). After stirring for an additional 0.5 h at ambient temperature, the mixture was stirred at 50° C. for 0.5 h. The mixture was cooled to ambient temperature, quenched with H₂O (0.14 mL) and 5M NaOH (0.035 mL). After stirring for 20 min, the mixture was passed through a bed of celite, rinsed with THF and concentrated in vacuo. The residue was purified via column chromatography (eluted with 30-70% EtOAc/Hex) to afford the desired alcohol 196 as a light purple solid in 68% yield. ¹H NMR (400 MHz, DMSO-d₆) δ 11.69 (s, 1H), 11.59 (s, 1H), 7.65 (s, 1H), 7.56-7.49 (m, 2H), 7.14 (dd, J=8.4, 1.8 Hz, 1H), 7.07 (s, 1H), 6.87 (dd, J=16.9, 2.0 Hz, 2H), 4.92 (t, J=5.4 Hz, 1H), 4.10 (t, J=5.1 Hz, 2H), 3.81 (q, J=5.2 Hz, 2H). Mass calculated for (C₁₈H₁₄BrClN₂O₂+H)⁺405.0, found 405.3.

### Compound 197: 2-((6′-Bromo-5-chloro-1H,1′H-[2,2′-biindol]-6-yl)oxy)ethyl Dimethylglycinate

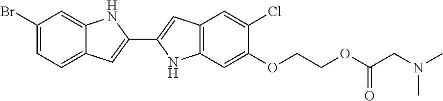 [SMILES: CN(C)CC(=O)OCCOc1cc2[nH]c(-c3cc4ccc(Br)cc4[nH]3)cc2cc1Cl]

Prepared according to general method XL from biindole alcohol 196 and N,N-dimethylglycine (14 mg, 28%). ¹H NMR (400 MHz, DMSO-d₆) δ 11.70 (s, 1H), 11.63 (s, 1H), 7.66 (s, 1H), 7.57-7.49 (m, 2H), 7.14 (dd, J=8.4, 1.8 Hz, 1H), 7.07 (s, 1H), 6.88 (dd, J=19.7, 2.1 Hz, 2H), 4.47 (t, J=4.4 Hz, 2H), 4.31 (t, J=4.5 Hz, 2H), 3.22 (s, 2H), 2.26 (s, 6H). Mass calculated for (C₂₂H₂₁BrClN₃O₃+H)⁺490.1, found 490.2.

### Compound 198: 2-((6′-Bromo-5-chloro-1H,1′H-[2,2′-biindol]-6-yl)oxy)ethyl Methanesulfonate

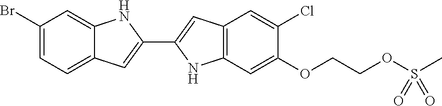 [SMILES: CS(=O)(=O)OCCOc1cc2[nH]c(-c3cc4ccc(Br)cc4[nH]3)cc2cc1Cl]

To a stirring solution of alcohol 196 (83 mg, 0.20 mmol) and pyridine (0.82 mL, 10.2 mmol) in THF (7 mL) was added MsCl (0.48 mL, 6.18 mmol). The resulting pink solution was stirred at ambient temperature for 21 h. The mixture was diluted with EtOAc and then successively washed with 1M HCl (3×) and brine (1×). The organics were dried over MgSO₄, filtered and concentrated in vacuo. The deep red residue was purified via column chromatography (eluted with 70-100% Et₂O/Hex) to afford the desired sulfonate 198 as a dark yellow solid in 42% yield. ¹H NMR (400 MHz, DMSO-d₆) δ 11.70 (s, 1H), 11.66 (s, 1H), 7.68 (s, 1H), 7.56-7.50 (m, 2H), 7.14 (dd, J=8.5, 1.8 Hz, 1H), 7.08 (s, 1H), 6.89 (dd, J=18.6, 2.0 Hz, 2H), 4.68-4.55 (m, 2H), 4.37 (dd, J=5.3, 3.2 Hz, 2H), 3.29 (s, 3H). Mass calculated for (C₁₉H₁₆BrClN₂O₄S+H)⁺483.0, found 483.2.

### Compound 199: 2-((6′-Bromo-5-chloro-1H,1′H-[2,2′-biindol]-6-yl)oxy)-N,N-dimethylethan-1-amine

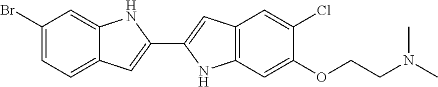 [SMILES: CN(C)CCOc1cc2[nH]c(-c3cc4ccc(Br)cc4[nH]3)cc2cc1Cl]

To a stirring solution of sulfonate 198 (20.8 mg, 0.043 mmol) in DMF (0.4 mL) was added an aqueous solution of dimethylamine (40 wt %, 0.145 mL, 1.29 mmol). The mixture was stirred at 50° C. for 16 h and then directly purified via column chromatography (eluted with 5-9% (5% NH₄OH/MeOH) in CH₂Cl₂) to afford the desired amine 199 as an off-white solid in 59% yield. ¹H NMR (400 MHz, DMSO-d₆) δ 11.71-11.66 (m, 1H), 11.60 (s, 1H), 7.65 (s, 1H), 7.56-7.49 (m, 2H), 7.14 (dd, J=8.4, 1.8 Hz, 1H), 7.05 (s, 1H), 6.92-6.81 (m, 2H), 4.16 (t, J=5.7 Hz, 2H), 2.73 (t, J=5.8 Hz, 2H), 2.29 (s, 6H). Mass calculated for (C₂₀H₁₉BrClN₃O+H)⁺ 432.0, found 431.9.

### Synthesis of Compound 200

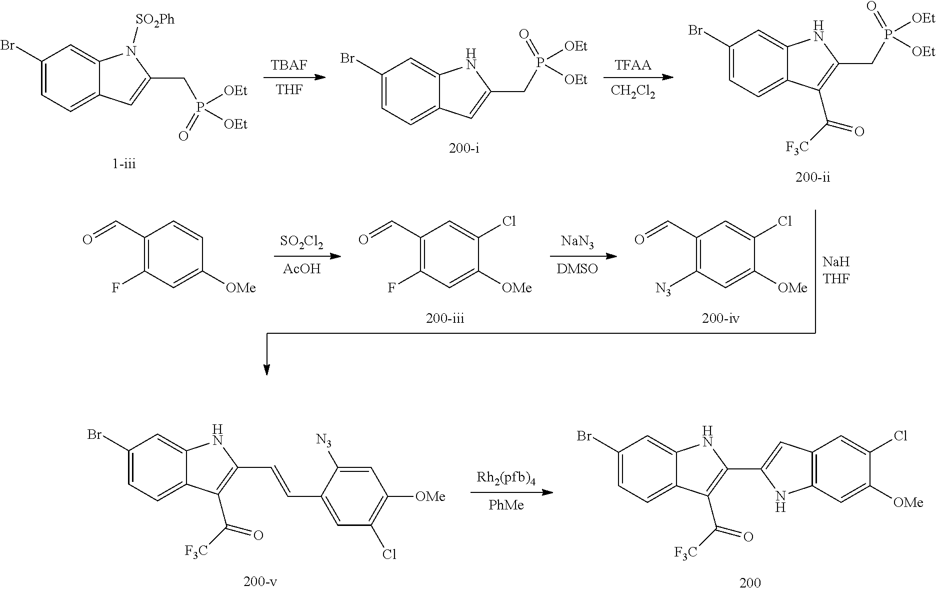

Intermediate 200-i: Diethyl ((6-bromo-1H-indol-2-yl)methyl)phosphonate

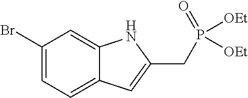 [SMILES: CCOP(=O)(Cc1cc2ccc(Br)cc2[nH]1)OCC]

To a stirring solution of phosphonate 1-iii (1.20 g, 2.47 mmol) in THF (15 mL) was added a 1M solution of TBAF in THF (5.0 mL, 5.0 mmol). After stirring at ambient temperature for 66 h, the mixture was stirred at 50° C. for 2 h. The mixture was concentrated in vacuo and purified via column chromatography (eluted with 40-80% EtOAc/Hex) to afford the unprotected phosphonate 200-i as a dark yellow solid in 59% yield. ¹H NMR (400 MHz, CDCl₃) δ 8.97 (s, 1H), 7.55-7.48 (m, 1H), 7.41 (d, J=8.4 Hz, 1H), 7.20 (dd, J=8.4, 1.7 Hz, 1H), 6.33 (t, J=2.5 Hz, 1H), 4.18-3.99 (m, 4H), 3.33 (d, J=20.8 Hz, 2H), 1.29 (t, J=7.0 Hz, 6H). Mass calculated for (C₁₃H₁₇BrNO₃P+H)⁺346.0, found 346.2.

### Intermediate 200-ii: Diethyl ((6-bromo-3-(2,2,2-trifluoroacetyl)-1H-indol-2-yl)methyl)phosphonate

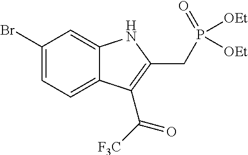 [SMILES: CCOP(=O)(Cc1[nH]c2cc(Br)ccc2c1C(=O)C(F)(F)F)OCC]

To a stirring solution of phosphonate 200-i (0.74 g, 2.13 mmol) in CH₂Cl₂ (20 mL) was added TFAA (1.0 mL, 7.19 mmol). After stirring at ambient temperature for 3 h, the mixture was concentrated in vacuo and co-evaporated with CH₂Cl₂ (5×) to afford ketone 200-ii as a light purple solid in quantitative yield. H NMR (400 MHz, CDCl₃) δ 11.09 (s, 1H), 7.83 (d, J=8.7 Hz, 1H), 7.47 (d, J=1.8 Hz, 1H), 7.38 (dd, J=8.8, 1.8 Hz, 1H), 4.24-4.09 (m, 4H), 4.03 (d, J=22.1 Hz, 2H), 1.33 (t, J=7.1 Hz, 6H). Mass calculated for (C₁₅H₁₆BrF₃NO₄P+H)⁺442.0, found 442.2.

### Intermediate 200-iii: 5-Chloro-2-fluoro-4-methoxybenzaldehyde

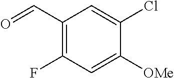 [SMILES: COc1cc(F)c(C=O)cc1Cl]

To a cold (0° C.) stirring solution of 2-fluoro-4-methoxybenzaldehyde (3.23 g, 21.0 mmol) in glacial AcOH (6 mL) was slowly added sulfuryl chloride (3.5 mL, 43.2 mmol). The mixture was stirred at ambient temperature for 16 h, poured onto ice water and stirred for 0.5 h. The resulting pale yellow suspension was extracted with CH₂Cl₂ (3×), washed with brine (Ix), dried (MgSO₄), filtered and concentrated in vacuo to afford aryl chloride 200-iii as a pale yellow solid in quantitative yield. The characterization data is in agreement with the literature (|J. Org. Chem. 2011, 76, 9519-9524).

### Intermediate 200-iv: 2-Azido-5-chloro-4-methoxybenzaldehyde

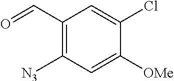 [SMILES: COc1cc(N=[N+]=[N-])c(C=O)cc1Cl]

A mixture was chloride 200-iv (2.8 g, 14.9 mmol), sodium azide (2.67 g, 41.1 mmol) and DMSO (25 mL) was stirring at 50° C. for 29 h. The mixture was cooled to ambient temperature, diluted with TBME (200 mL) and successively washed with H₂O (3×), saturated NH₄Cl (1×), and brine (1×). The aqueous washes were combined and extracted with TBME (Ix). The combined organics was dried (MgSO₄), filtered and concentrated in vacuo. The yellow residue was purified by trituration in CH₂Cl₂ to afford aryl azide 200-iv as a light orange solid in 38% yield. ¹H NMR (400 MHz, CDCl₃) δ 10.19 (s, 1H), 7.93 (s, 1H), 6.72 (s, 1H), 4.05 (s, 3H).

### Intermediate 200-v: (E)-1-(2-(2-Azido-5-chloro-4-methoxystyryl)-6-bromo-1 H-indol-3-yl)-2,2,2-trifluoroethan-1-one

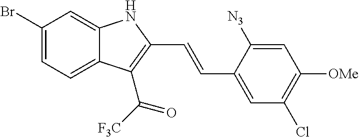 [SMILES: COc1cc(N=[N+]=[N-])c(/C=C/c2[nH]c3cc(Br)ccc3c2C(=O)C(F)(F)F)cc1Cl]

Sodium hydride (60% dispersion, 0.025 g, 0.62 mmol) was added to a cold (0° C.) stirring solution of phosphonate 200-ii (0.114 g, 0.25 mmol) in THF (3.0 mL) under N₂. The resulting mixture was stirred for 15 min and then azide 200-iv (0.057 g, 0.27 mmol) was added in one portion. The resulting deep green mixture was stirred cold for 5 min then at ambient temperature for 5 h. The dark yellow mixture was concentrated in vacuo, co-evaporated with MeOH (3×) and dissolved in minimal MeOH. After standing for 4 d, vinyl indole 200-v was collected as orange needles in 19% yield. ¹H NMR (400 MHz, CDCl₃) δ 9.07 (s, 1H), 7.95-7.82 (m, 2H), 7.77 (s, 1H), 7.61 (d, J=1.8 Hz, 1H), 7.46-7.36 (m, 2H), 6.70 (s, 1H), 4.01 (s, 3H). Mass calculated for (C₁₉H₁₁BrClF₃N₄O₂−H)⁻ 497.0, found 497.5.

### Compound 200: 1-(6-Bromo-5′-chloro-6′-methoxy-1H, 1′H-[2,2′-biindol]-3-yl)-2,2,2-trifluoroethan-1-one

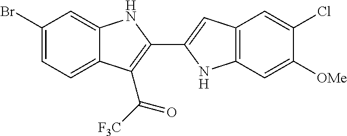 [SMILES: COc1cc2[nH]c(-c3[nH]c4cc(Br)ccc4c3C(=O)C(F)(F)F)cc2cc1Cl]

A mixture of vinyl indole 200-v (20 mg, 0.039 mmol), rhodium(II) perfluorobutyrate dimer (1.0 mg, 0.95 μmol) and toluene (0.3 mL) was stirred at 80° C. under N₂ for 16 h. The mixture was cooled to ambient temperature, concentrated in vacuo and purified by column chromatography with 10-70% Et-₂O/hexanes to afford the desired protected biindole 200 as an orange solid in 74% yield. ¹H NMR (400 MHz, CDCl₃) δ 11.98 (s, 1H), 9.22 (s, 1H), 7.88 (d, J=8.8 Hz, 1H), 7.74-7.61 (m, 2H), 7.45 (dd, J=8.8, 1.8 Hz, 1H), 7.07 (s, 2H), 4.01 (s, 3H). Mass calculated for (C₁₉H₁₁BrClF₃N₂O₂−H)⁻ 469.0, found 469.5.

### Synthesis of Compound 201

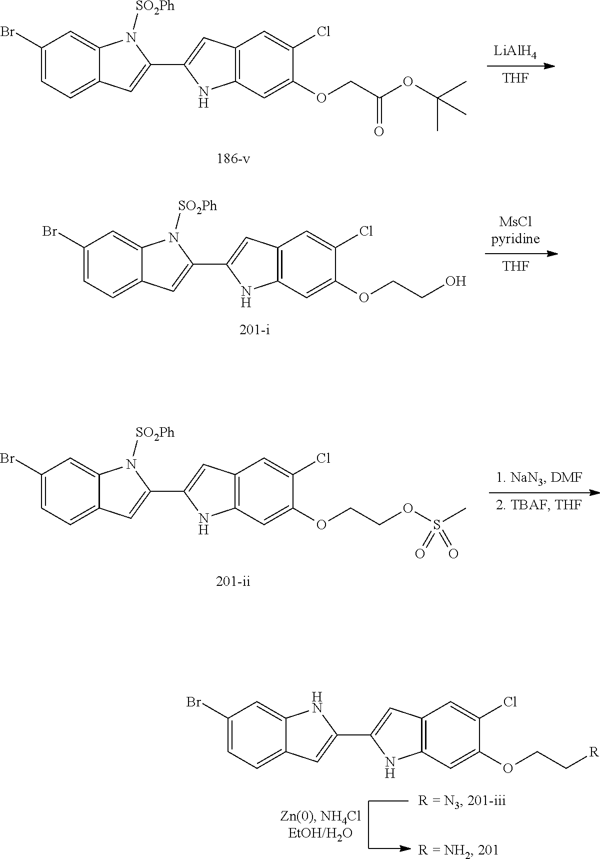

### Intermediate 201-i: 2-((6′-Bromo-5-chloro-1′-(phenylsulfonyl)-1H, 1′H-[2,2′-biindol]-6-yl)oxy)ethan-1-ol

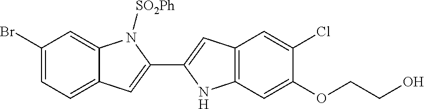

Lithium aluminum hydride (111 mg, 2.92 mmol) was added to a cold (0° C.) stirring solution of biindole t-butyl ester 186-v (355 mg, 0.576 mmol) in THF (12 mL). After stirring for 0.5 h at ambient temperature, the mixture cooled to 0° C. and carefully quenched with H₂O (0.2 mL) and 5M NaOH (0.15 mL). After stirring for 20 min, the mixture was passed through a bed of celite, rinsed with THF and concentrated in vacuo. The residue was purified via column chromatography (eluted with 20-80% EtOAc/Hex) to afford the desired alcohol 201-i as a light green film in 97% yield. ¹H NMR (400 MHz, CDCl₃) δ 8.95 (s, 1H), 8.56 (d, J=1.6 Hz, 1H), 7.64 (d, J=3.1 Hz, 1H), 7.53-7.33 (m, 7H), 7.07 (s, 1H), 6.75 (s, 1H), 6.54 (d, J=2.0 Hz, 1H), 5.57 (s, 1H), 4.25 (t, J=4.5 Hz, 2H), 4.08 (d, J=5.2 Hz, 2H). Mass calculated for (C₂₄H₁₈BrClN₂O₄S+H)⁺545.0, found 545.4.

### Intermediate 201-ii: 2-((6′-Bromo-5-chloro-1′-(phenylsulfonyl)-1H, 1′H-[2,2′-biindol]-6-yl)oxy)ethyl Methanesulfonate

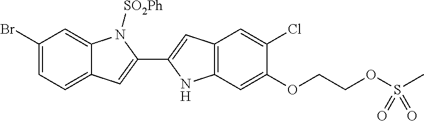

To a stirring solution of alcohol 201-i (302 mg, 0.553 mmol) and pyridine (1.1 mL, 13.7 mmol) in THF (9 mL) was added MsCl (0.86 mL, 11.1 mmol). The resulting red orange solution was stirred at ambient temperature for 18 h. The mixture was diluted with EtOAc and then successively washed with 1M HCl (3×), sat. CuSO₄ (2×) and brine (2×). The organics were dried over MgSO₄, filtered and concentrated in vacuo. The deep red residue was purified via column chromatography (eluted with 50-90% Et₂O/Hex) to afford the desired sulfonate 201-ii as an off-white solid in 31% yield. ¹H NMR (400 MHz, CDCl₃) δ 8.96 (s, 1H), 8.56 (s, 1H), 7.65 (s, 1H), 7.57-7.31 (m, 7H), 7.06 (s, 1H), 6.77 (s, 1H), 6.55 (s, 1H), 4.72 (t, J=4.4 Hz, 2H), 4.43-4.36 (m, 2H), 3.22 (s, 3H). Mass calculated for (C₂₅H₂₀BrClN₂O₆S₂+H)⁺623.0, found 623.3.

### Intermediate 201-iii: 6-(2-Azidoethoxy)-6′-bromo-5-chloro-1H, 1′H-2,2′-biindole

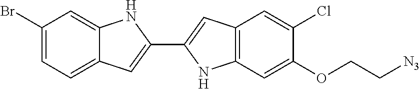 [SMILES: [N-]=[N+]=NCCOc1cc2[nH]c(-c3cc4ccc(Br)cc4[nH]3)cc2cc1Cl]

A mixture of sulfonate 201-ii (105 mg, 0.168 mmol), sodium azide (100 mg, 1.54 mmol) and DMF (3 mL) was stirred at 60° C. After 3 h, the mixture was co-evaporated with PhMe (2×) and then THF (1×). The residue was dissolved in THF (3 mL), treated with a 1M solution of TBAF in THF (2.5 mL, 2.5 mmol) and stirred at reflux for 20 h. The mixture was concentrated in vacuo and purified by column chromatography with 40-80% Et₂O/hexanes to afford the desired azido biindole 201-iii as an off-white solid in 51% yield. ¹H NMR (400 MHz, DMSO-d₆) δ 11.70 (d, J=2.1 Hz, 1H), 11.64 (d, J=2.2 Hz, 1H), 7.68 (s, 1H), 7.53 (d, J=8.4 Hz, 2H), 7.14 (dd, J=8.3, 1.8 Hz, 1H), 7.07 (s, 1H), 6.94-6.83 (m, 2H), 4.33-4.22 (m, 2H), 3.72 (t, J=4.7 Hz, 2H). Mass calculated for (C₁₈H₁₃BrClN₅O−H)⁻ 428.0, found 428.5.

### Compound 201: 2-((6′-Bromo-5-chloro-1H,1′H-[2,2′-biindol]-6-yl)oxy)ethan-1-amine

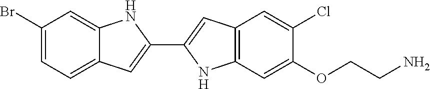 [SMILES: NCCOc1cc2[nH]c(-c3cc4ccc(Br)cc4[nH]3)cc2cc1Cl]

A mixture of azide 201-iii (29 mg, 0.067 mmol), Zn dust (25 mg, 0.378 mmol), NH₄Cl (38 mg, 0.705 mmol), EtOH (0.38 mL) and H₂O (0.12 mL) was stirred at ambient temperature. After 17 h, the mixture was passed through a bed of Celite, rinsed with EtOAc and concentrated in vacuo. The residue was purified by column chromatography with 10-40% MeOH/CH₂Cl₂ to afford the desired amino biindole 201 as an off-white solid in 67% yield. ¹H NMR (400 MHz, DMSO-d₆) δ 11.75 (d, J=2.1 Hz, 1H), 11.67 (d, J=2.2 Hz, 1H), 7.67 (s, 1H), 7.53 (d, J=8.6 Hz, 2H), 7.14 (dd, J=8.4, 1.8 Hz, 1H), 7.08 (s, 1H), 6.88 (dt, J=15.6, 3.1 Hz, 2H), 5.11 (br s, 2H), 4.13 (t, J=5.5 Hz, 2H), 3.10 (s, 2H). Mass calculated for (C₁₈H₁₅BrClN₃O+H)⁺404.0, found 403.9.

### Synthesis of Compound 202

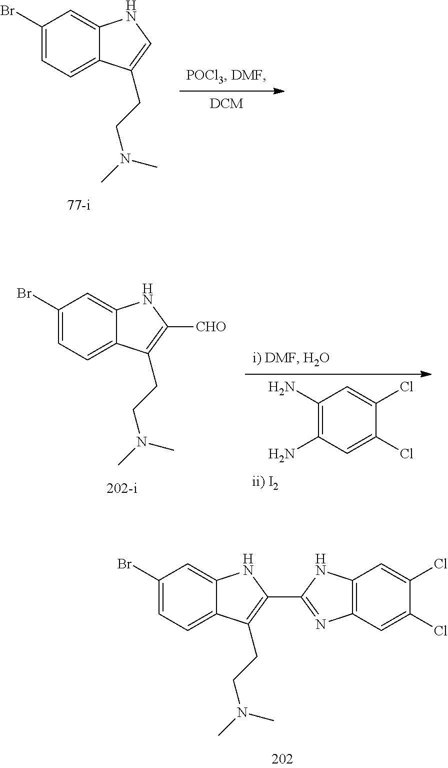

### Compound 202: 2-(6-Bromo-2-(5,6-dichloro-1H-benzo[d]imidazol-2-yl)-1H-indol-3-yl)-N,N-dimethylethan-1-amine

POCl₃ (1.0 mL, 10.9 mmol) was added to DMF (2.0 mL) at 0° C. under Ar and the mixture was allowed to warm to rt. The resulting mixture was then added to a stirred solution of 77-i (1.05 g, 3.93 mmol)) in DMF/DCM (1/1, 16 mL) at 0° C. under Ar gradually. The mixture was heated with μwave at 100° C. for 1 h and then sat. aqueous solution of NaHCO₃ (50 mL) was slowly added. The mixture was extracted with EtOAc and the organic layer was washed with brine, dried over anhydrous Na₂SO₄, filtered and then concentrated under reduced pressure. The residue was partially purified by silica gel chromatography, eluting with MeOH/DCM gradient, to provide the corresponding aldehyde intermediate 202-i (745 mg).

A mixture of intermediate 202-i (˜220 mg, 0.75 mmol) and 4,5-dichlorobenzene-1,2-diamine (140 mg, 0.79 mmol) in DMF/H₂O (9/1, 5 mL) was heated at 50° C. in an open vial for 17 h. I₂ (100 mg, 0.65 mmol) was added and the mixture was stirred at rt for 20 min. The mixture was diluted with EtOAc (100 mL) and the resulting mixture was washed with brine (20 mL), dried over anhydrous Na₂SO₄, filtered and then concentrated under reduced pressure. The residue was purified by silica gel chromatography, eluting with MeOH (5% aqueous NH₄OH)/DCM gradient, to provide compound 202 (26 mg, 8%). ¹H NMR (400 MHz, DMSO-d₆) δ 11.82 (s, 1H), 7.91 (s, 2H), 7.63 (d, J=8.6 Hz, 1H), 7.61 (d, J=1.8 Hz, 1H), 7.20 (dd, J=8.5, 1.8 Hz, 1H), 3.22 (t, J=5.8 Hz, 2H), 2.78 (t, J=5.8 Hz, 2H), 2.44 (s, 6H). Mass calculated for (C₁₉H₁₇BrCl₂N₄+H)⁺451.0, found 450.8.

### Synthesis of Compound 203

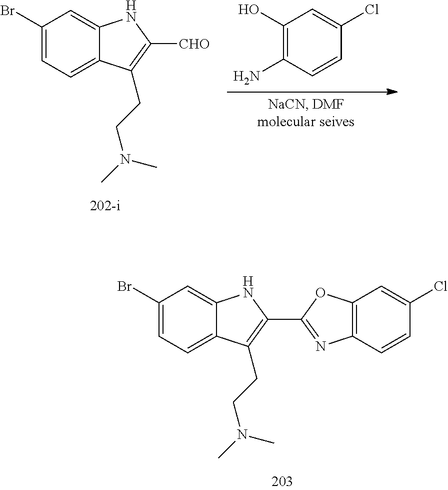

### Compound 203: 2-(6-Bromo-2-(6-chlorobenzo[d]oxazol-2-yl)-1H-indol-3-yl)-N,N-dimethylethan-1-amine

A mixture of intermediate 202-i (50 mg, 0.17 mmol), 2-amino-5-chlorophenol (30 mg, 0.21 mmol), NaCN (10 mg, 0.20 mmol) and 4 Å molecular sieves (200 mg) in DMF (2 mL) was heated at 80° C. in an open vial for 4 h and then concentrated under reduced pressure. The residue was purified by silica gel chromatography, eluting with MeOH (5% aqueous NH₄OH)/DCM gradient, to provide compound 203 (8 mg, 11%). ¹H NMR (400 MHz, DMSO-d₆) δ 12.23 (s, 1H), 8.00 (d, J=1.9 Hz, 1H), 7.83 (d, J=8.5 Hz, 1H), 7.71 (d, J=8.6 Hz, 1H), 7.63 (d, J=1.7 Hz, 1H), 7.50 (dd, J=8.5, 2.0 Hz, 1H), 7.26 (dd, J=8.6, 1.8 Hz, 1H), 3.46-3.41 (m, 2H), 2.75-2.66 (m, 2H), 2.38 (s, 6H). Mass calculated for (C₁₉H₁₇BrClN₃O+H)⁺418.0, found 417.9.

### Compound 204: (6-Bromo-5′-chloro-1H,1′H-[2,2′-biindole]-3-carbonyl)glycine

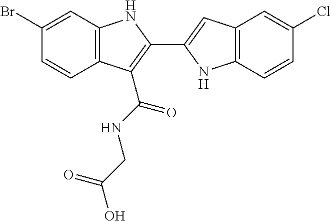 [SMILES: O=C(O)CNC(=O)c1c(-c2cc3cc(Cl)ccc3[nH]2)[nH]c2cc(Br)ccc12]

Prepared according to general method XIV from intermediate 20-i and glycine (26 mg, 45%). ¹H NMR (400 MHz, DMSO-d₆) δ 12.62 (s, 1H), 12.33 (s, 1H), 8.58 (t, J=5.9 Hz, 1H), 7.84 (d, J=8.6 Hz, 1H), 7.72 (d, J=2.0 Hz, 1H), 7.63 (d, J=1.8 Hz, 1H), 7.60 (d, J=8.7 Hz, 1H), 7.32 (dd, J=8.6, 1.8 Hz, 1H), 7.18 (d, J=1.5 Hz, 1H), 7.16 (dd, J=8.7, 2.1 Hz, 1H), 4.04 (d, J=5.7 Hz, 2H). Mass calculated for (C₁₉H₁₃BrClN₃O₃−H)⁻ 444.0, found 443.9.

### Compound 205: 3-(6-Bromo-5′-chloro-1H,1′H-[2,2′-biindole]-3-carboxamido)propanoic Acid

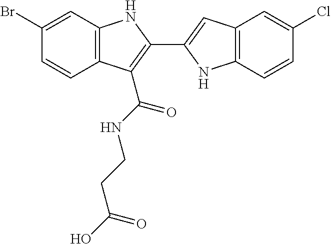 [SMILES: O=C(O)CCNC(=O)c1c(-c2cc3cc(Cl)ccc3[nH]2)[nH]c2cc(Br)ccc12]

Prepared according to general method XIV from intermediate 20-i and 3-aminopropanoic acid (29 mg, 49%). ¹H NMR (400 MHz, DMSO-d₆) δ 12.88-12.42 (m, 2H), 8.41 (bs, 1H), 7.78 (d, J=8.6 Hz, 1H), 7.71 (d, J=2.0 Hz, 1H), 7.63 (d, J=1.8 Hz, 1H), 7.61 (d, J=8.9 Hz, 1H), 7.27 (dd, J=8.6, 1.8 Hz, 1H), 7.18-7.13 (m, 2H), 3.60-3.57 (m, 2H), 2.57 (t, J=6.9 Hz, 2H). Mass calculated for (C₂₀H₁₅BrClN₃O₃−H)⁻ 458.0, found 457.9.

### Compound 206: N-(2-Aminoethyl)-6-bromo-5′-chloro-1H,1′H-[2,2′-biindole]-3-carboxamide

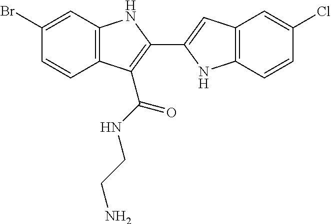 [SMILES: NCCNC(=O)c1c(-c2cc3cc(Cl)ccc3[nH]2)[nH]c2cc(Br)ccc12]

Prepared according to general method XIV from intermediate 20-i and ethane-1,2-diamine (41 mg, 73%). ¹H NMR (400 MHz, DMSO-d₆) δ 8.37 (bs, J=8.6 Hz, 2H), 7.82 (d, J=8.6 Hz, 1H), 7.73 (d, J=2.0 Hz, 1H), 7.65 (d, J=1.7 Hz, 1H), 7.58 (d, J=8.7 Hz, 1H), 7.32 (dd, J=8.6, 1.8 Hz, 1H), 7.21-7.14 (m, 2H), 3.58-3.55 (m, 2H), 3.00 (t, J=6.3 Hz, 2H), 1.01 (d, J=6.5 Hz, 2H). Mass calculated for (C₁₉H₁₆BrClN₄O+H)⁺431.0, found 430.8.

### General Method XLII

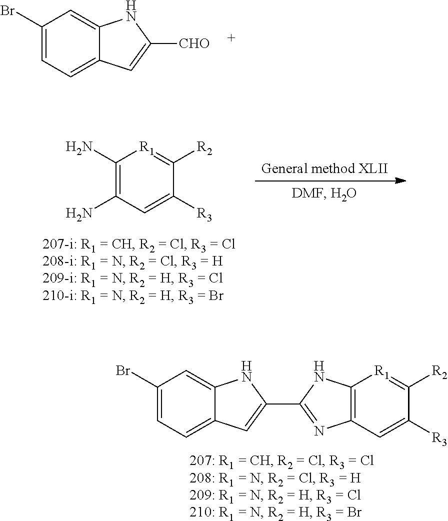

A mixture of either 207-i, 208-i, 209-i, or 210-i (1 mmol) and 6-bromo-1H-indole-2-carbaldehyde (1.1 mmol) in DMF/H₂O (9/1, 7 mL) was heated at 100° C. in an open vial for 1-5 d. Upon completion of the reaction (HPLC analysis), the mixture was concentrated under reduced pressure. For compound 207, the residue was purified by silica gel chromatography, eluting with EtOAc/hexanes gradient, to provide compound 207. For compounds 208-210, the residue was triturated with MeOH (10 mL) and the solid was collected by filtration to give the desired adduct.

### Compound 207: 2-(6-Bromo-1H-indol-2-yl)-5,6-dichloro-1H-benzo[d]imidazole

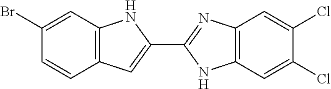 [SMILES: Clc1cc2nc(-c3cc4ccc(Br)cc4[nH]3)[nH]c2cc1Cl]

Prepared according to general method XLII from 207-i (1.26 g, 74%). ¹H NMR (400 MHz, DMSO-d₆) δ 13.40 (s, 1H), 12.23 (s, 1H), 7.87 (s, 2H), 7.68-7.59 (m, 2H), 7.29 (s, 1H), 7.20 (dd, J=8.4, 1.8 Hz, 1H). Mass calculated for (C₁₅H₈BrCl₂N₃+H)⁺379.9, found 379.8.

### Compound 208: 2-(6-Bromo-1H-indol-2-yl)-5-chloro-1H-imidazo[4,5-b]pyridine

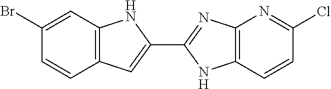 [SMILES: Clc1ccc2[nH]c(-c3cc4ccc(Br)cc4[nH]3)nc2n1]

Prepared according to general method XLII from 208-i (16 mg, 10%). ¹H NMR (400 MHz, DMSO-d₆) (present as a mixture of tautomer, major tautomer) δ 13.57 (s, 1H), 12.30 (s, 1H), 8.03 (d, J=8.3 Hz, 1H), 7.69-7.63 (m, 2H), 7.37-7.31 (m, 2H), 7.22 (s, 1H). Mass calculated for (C₁₄H₈BrClN₄+H)⁺347.0, found 347.0.

### Compound 209: 2-(6-Bromo-1H-indol-2-yl)-6-chloro-1H-imidazo[4,5-b]pyridine

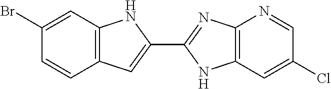 [SMILES: Clc1cnc2nc(-c3cc4ccc(Br)cc4[nH]3)[nH]c2c1]

Prepared according to general method XLII from 209-i (76 mg, 49%). ¹H NMR (400 MHz, DMSO-d₆) (present as a mixture of tautomer, major tautomer) δ 13.94 (s, 1H), 12.26 (s, 1H), 8.36 (d, J=2.2 Hz, 1H), 8.21 (d, J=2.2 Hz, 1H), 7.69-7.62 (m, 2H), 7.37 (s, 1H), 7.22 (s, 1H). Mass calculated for (C₁₄H₈BrClN₄+H)⁺347.0, found 346.9.

### Compound 210: 6-Bromo-2-(6-bromo-1H-indol-2-yl)-1H-imidazo[4,5-b]pyridine

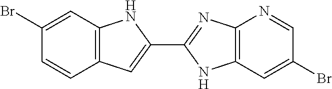 [SMILES: Brc1ccc2cc(-c3nc4ncc(Br)cc4[nH]3)[nH]c2c1]

Prepared according to general method XLII from 210-i (98 mg, 56%). ¹H NMR (400 MHz, DMSO-d₆) (present as a mixture of tautomer, major tautomer) δ 13.94 (s, 1H), 12.27 (s, 1H), 8.42 (d, J=2.1 Hz, 1H), 8.33 (d, J=2.1 Hz, 1H), 7.69-7.62 (m, 2H), 7.37 (s, 1H), 7.22 (s, 1H). Mass calculated for (C₁₄H₈Br₂N₄+H)⁺392.9, found 392.8.

### General Method XLIII

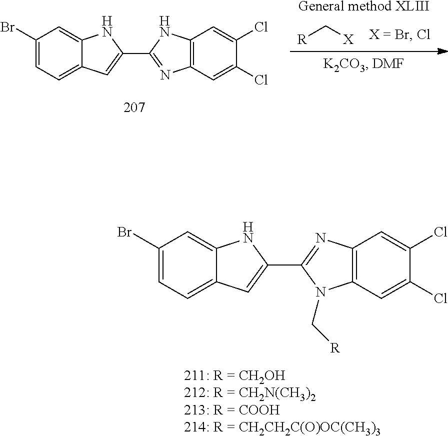

A mixture of compound 207 (1 mmol), K₂CO₃ (2 mmol) and the corresponding alkyl halide (1.1 mmol) in DMF (8 mL) was heated at 100° C. for 3-23 h. Upon completion of the reaction (HPLC analysis), the mixture was concentrated under reduced pressure. The residue was purified by silica gel chromatography, eluting with either EtOAc/hexanes or MeOH/DCM gradient, to provide the desired adduct.

### Compound 211: 2-(2-(6-Bromo-1H-indol-2-yl)-5,6-dichloro-1H-benzo[d]imidazol-1-yl)ethan-1-ol

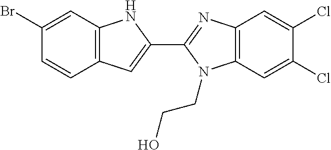 [SMILES: OCCn1c(-c2cc3ccc(Br)cc3[nH]2)nc2cc(Cl)c(Cl)cc21]

Prepared according to general method XLIII from 207 and 2-bromoethanol (11 mg, 20%). ¹H NMR (400 MHz, DMSO-d₆) δ 12.13 (s, 1H), 8.08 (s, 1H), 7.95 (s, 1H), 7.67 (d, J=1.7 Hz, 1H), 7.65 (d, J=8.5 Hz, 1H), 7.30 (s, 1H), 7.22 (dd, J=8.5, 1.8 Hz, 1H), 5.12 (bs, 1H), 4.63 (t, J=5.3 Hz, 2H), 3.90 (bs, 2H). Mass calculated for (C₁₇H₁₂BrCl₂N₃O+H)⁺424.0, found 423.8.

### Compound 212: 2-(2-(6-Bromo-1H-indol-2-yl)-5,6-dichloro-1H-benzo[d]imidazol-1-yl)-N,N-dimethylethan-1-amine

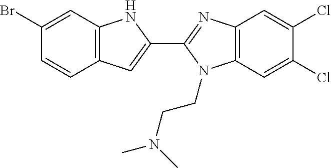 [SMILES: CN(C)CCn1c(-c2cc3ccc(Br)cc3[nH]2)nc2cc(Cl)c(Cl)cc21]

Prepared according to general method XLIII from 207 and 2-chloro-N,N-dimethylethan-1-amine hydrochloride (12 mg, 20%). ¹H NMR (400 MHz, DMSO-d₆) δ 12.32 (s, 1H), 8.08 (s, 1H), 7.93 (s, 1H), 7.66 (d, J=1.7 Hz, 1H), 7.64 (d, J=8.6 Hz, 1H), 7.22 (s, 1H), 7.19 (dd, J=8.5, 1.8 Hz, 1H), 4.70 (t, J=6.4 Hz, 2H), 2.69 (t, J=6.4 Hz, 2H), 2.19 (s, 6H). Mass calculated for (C₁₉H₁₇BrCl₂N₄+H)⁺451.0, found 450.8.

### Compound 213: 2-(2-(6-Bromo-1H-indol-2-yl)-5,6-dichloro-1H-benzo[d]imidazol-1-yl)acetic Acid

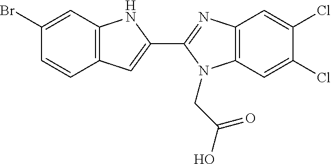 [SMILES: O=C(O)Cn1c(-c2cc3ccc(Br)cc3[nH]2)nc2cc(Cl)c(Cl)cc21]

Prepared according to general method XLIII from 207 and 2-bromoacetic acid (13 mg, 22%). ¹H NMR (400 MHz, DMSO-d₆) δ 12.33 (s, 1H), 7.92 (s, 2H), 7.66 (s, 1H), 7.61 (d, J=8.5 Hz, 1H), 7.20 (dd, J=8.4, 1.6 Hz, 1H), 7.10 (s, 1H), 4.88 (s, 2H). Mass calculated for (C₁₇H₁₀BrCl₂N₃O₂−H)⁻ 435.9, found 435.8.

### Compound 214: tert-Butyl 3-(2-(6-bromo-1H-indol-2-yl)-5,6-dichloro-1H-benzo[d]imidazol-1-yl)propanoate

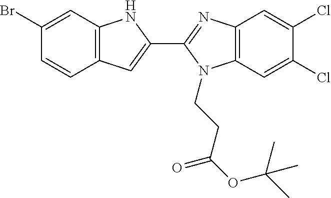 [SMILES: CC(C)(C)OC(=O)CCn1c(-c2cc3ccc(Br)cc3[nH]2)nc2cc(Cl)c(Cl)cc21]

Prepared according to general method XLIII from 207 and tert-butyl 3-bromopropanoate (15 mg, 11%). ¹H NMR (400 MHz, DMSO-d₆) δ 13.35 (s, 1H), 8.00-7.72 (m, 3H), 7.67 (d, J=8.5 Hz, 1H), 7.38 (s, 1H), 7.27 (dd, J=8.4, 1.7 Hz, 1H), 5.10 (t, J=6.8 Hz, 2H), 2.76 (t, J=6.8 Hz, 2H), 1.23 (s, 9H). Mass calculated for (C₂₂H₂₀BrCl₂N₃O₂+H)⁺508.0, found 507.8.

### Synthesis of Compound 215

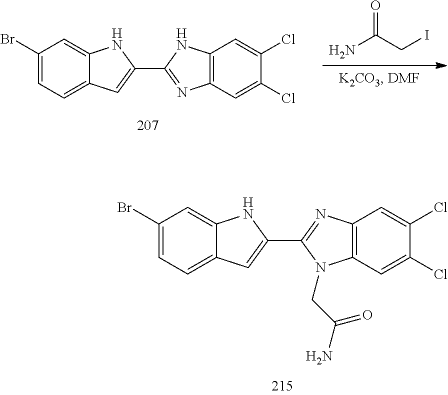

### Compound 215: 2-(2-(6-Bromo-1H-indol-2-yl)-5,6-dichloro-1H-benzo[d]imidazol-1-yl)acetamide

A mixture of compound 207 (50 mg, 0.13 mmol), K₂CO₃ (36 mg, 0.26 mmol) and 2-iodoacetamide (26 mg, 0.14 mmol) in DMF (1.5 mL) was stirred at rt for 2 h and then concentrated under reduced pressure. The residue was purified by silica gel chromatography, eluting with EtOAc/hexanes, to provide compound 215 (25 mg, 43%). ¹H NMR (400 MHz, DMSO-d₆) δ 12.21 (s, 1H), 8.10 (s, 1H), 7.97 (s, 1H), 7.93 (bs, 1H), 7.67 (s, 1H), 7.64 (d, J=8.5 Hz, 1H), 7.54 (bs, 1H), 7.22 (dd, J=8.5, 1.8 Hz, 1H), 7.02 (d, J=1.6 Hz, 1H), 5.23 (s, 2H). Mass calculated for (C₁₇H₁₁BrCl₂N₄O+H)⁺437.0, found 436.8.

### General Method XLIV

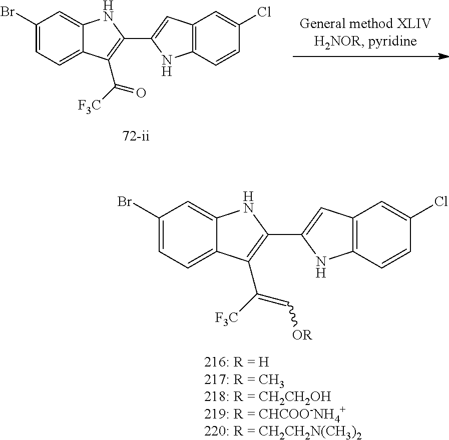

A mixture of compound 72-ii (1 mmol) and the corresponding amine (4-5 mmol) in pyridine (7 mL) was heated at 50° C. for 1-2 d. Upon completion of the reaction (HPLC analysis), the mixture was concentrated under reduced pressure. The residue was purified by silica gel chromatography, eluting with either MeOH/DCM or MeOH (5% aqueous NH₄OH)/DCM gradient, to provide the desired adduct.

### Compound 216: 1-(6-Bromo-6′-chloro-1H,1′H-[2,2′-biindol]-3-yl)-2,2,2-trifluoroethan-1-one Oxime

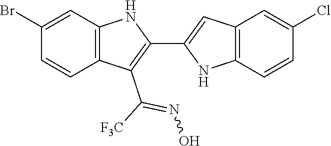 [SMILES: ON=C(c1c(-c2cc3cc(Cl)ccc3[nH]2)[nH]c2cc(Br)ccc12)C(F)(F)F]

Prepared according to general method XLIV from 72-ii and hydroxylamine hydrochloride (44 mg, 62%). Present as a mixture of E/Z isomers. Major isomer: H NMR (400 MHz, DMSO-d₆) δ 12.78 (s, 1H), 12.09 (s, 1H), 11.58 (s, 1H), 7.73-7.66 (m, 2H), 7.50 (d, J=8.6 Hz, 1H), 7.28 (s, 2H), 7.17 (dd, J=8.6, 2.1 Hz, 1H), 6.64 (s, 1H). Mass calculated for (C₁₈H₁₀BrClF₃N₃O−H)⁻ 456.0, found 455.8.

### Compound 217: 1-(6-Bromo-6′-chloro-1H,1′H-[2,2′-biindol]-3-yl)-2,2,2-trifluoroethan-1-one O-methyl Oxime

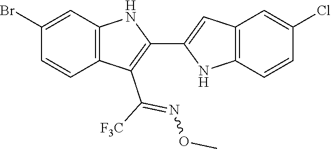 [SMILES: CON=C(c1c(-c2cc3cc(Cl)ccc3[nH]2)[nH]c2cc(Br)ccc12)C(F)(F)F]

Prepared according to general method XLIV from 72-ii and methoxyamine hydrochloride (52 mg, 64%). Present as a mixture of E/Z isomers. Major isomer: ¹H NMR (400 MHz, DMSO-d₆) δ 12.21 (s, 1H), 11.70 (s, 1H), 7.75-7.66 (m, 2H), 7.51-7.44 (m, 2H), 7.32 (dd, J=8.5, 1.8 Hz, 1H), 7.19 (dd, J=8.6, 2.2 Hz, 1H), 6.73 (s, 1H), 4.14 (s, 3H). Mass calculated for (C₁₉H₁₂BrClF₃N₃O−H)⁻ 470.0, found 469.9.

### Compound 218: 1-(6-Bromo-6′-chloro-1H,1′H-[2,2′-biindol]-3-yl)-2,2,2-trifluoroethan-1-one O-(2-hydroxyethyl) Oxime

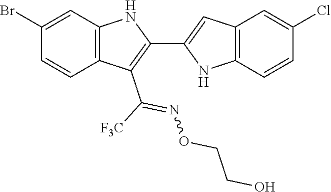 [SMILES: OCCON=C(c1c(-c2cc3cc(Cl)ccc3[nH]2)[nH]c2cc(Br)ccc12)C(F)(F)F]

Prepared according to general method XLIV from 72-ii and 2-(aminooxy)ethan-1-ol (49 mg, 68%). Present as a mixture of E/Z isomers. Major isomer: H NMR (400 MHz, DMSO-d₆) δ 12.19 (s, 1H), 11.57 (s, 1H), 7.72-7.66 (m, 2H), 7.50 (d, J=8.7 Hz, 1H), 7.46 (d, J=8.5 Hz, 1H), 7.31 (dd, J=8.6, 1.8 Hz, 1H), 7.19 (dd, J=8.6, 2.1 Hz, 1H), 6.79 (s, 1H), 4.97 (t, J=5.3 Hz, 1H), 4.38 (t, J=5.0 Hz, 2H), 3.74 (q, J=5.2 Hz, 2H). Mass calculated for (C₂₀H₁₄BrClF₃N₃O₂−H)⁻ 500.0, found 499.9.

### Compound 219: Ammonium 2-(((1-(6-bromo-5′-chloro-1H,1′H-[2,2′-biindol]-3-yl)-2,2,2-trifluoroethylidene)amino)oxy)acetate

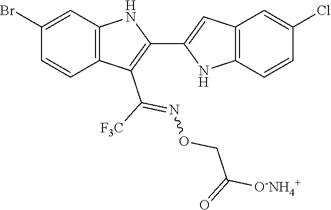

Prepared according to general method XLIV from 72-ii and 2-(aminooxy)acetic acid hydrochloride. The crude product was purified by silica gel chromatography, eluting with MeOH (5% aqueous NH₄OH)/DCM gradient, to provide the desired adduct (7.5 mg, 16%). Present as a mixture of E/Z isomers. Major isomer: ¹H NMR (400 MHz, Methanol-d₄) δ 7.82 (d, J=8.7 Hz, 1H), 7.63 (d, J=1.7 Hz, 1H), 7.56 (d, J=2.0 Hz, 1H), 7.41 (d, J=8.5 Hz, 1H), 7.25 (dd, J=8.5, 1.7 Hz, 1H), 7.12 (dd, J=8.7, 2.0 Hz, 1H), 6.93 (s, 1H), 4.80 (s, 2H). Mass calculated for (C₂₀H₁₅BrClF₃N₄O₃−NH₄)⁻ 512.0, found 511.9.

### Compound 220: 1-(6-Bromo-5′-chloro-1H,1′H-[2,2′-biindol]-3-yl)-2,2,2-trifluoroethan-1-one O-(2-(dimethylamino)ethyl)oxime

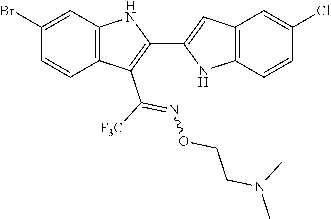 [SMILES: CN(C)CCON=C(c1c(-c2cc3cc(Cl)ccc3[nH]2)[nH]c2cc(Br)ccc12)C(F)(F)F]

Prepared according to general method XLIV from 72-ii and 2-(aminooxy)-N,N-dimethylethan-1-amine. The crude product was purified by silica gel chromatography, eluting with MeOH (5% aqueous NH₄OH)/DCM gradient, to provide the desired adduct (21 mg, 44%). Present as a mixture of E/Z isomers. Major isomer: ¹H NMR (400 MHz, Methanol-d₄) δ 7.66 (d, J=1.8 Hz, 1H), 7.59 (d, J=2.0 Hz, 1H), 7.47-7.41 (m, 2H), 7.29 (dd, J=8.6, 1.8 Hz, 1H), 7.16 (dd, J=8.6, 2.0 Hz, 1H), 6.73 (s, 1H), 4.45 (t, J=5.6 Hz, 2H), 2.70 (t, J=5.6 Hz, 2H), 2.30 (s, 6H). Mass calculated for (C₂₂H₁₉BrClF₃N₄O+H)⁺527.0, found 526.9.

### Synthesis of Compound 221

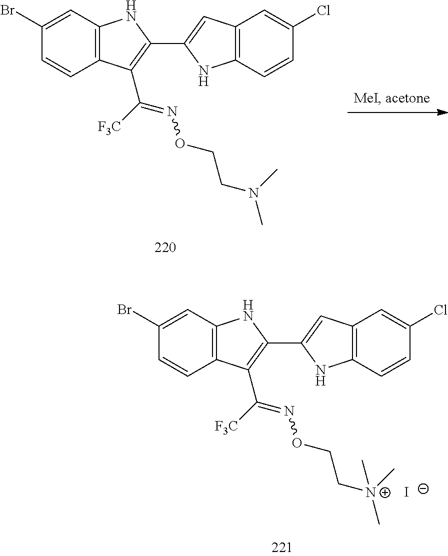 [SMILES: CN(C)CCON=C(c1c(-c2cc3cc(Cl)ccc3[nH]2)[nH]c2cc(Br)ccc12)C(F)(F)F ; "MeI, acetone" ; C[N+](C)(C)CCON=C(c1c(-c2cc3cc(Cl)ccc3[nH]2)[nH]c2cc(Br)ccc12)C(F)(F)F ; [I-]]

### Compound 221: 2-(((1-(6-Bromo-5′-chloro-1H,1′H-[2,2′-biindol]-3-yl)-2,2,2-trifluoroethylidene)amino)oxy)-N,N,N-trimethylethan-1-aminium Iodide

MeI (30 μL, 0.48 mmol) was added to a stirred solution of compound 220 (190 mg, 0.36 mmol) in acetone (8 mL). The mixture was stirred at rt for 90 min and then concentrated. The residue was triturated with Et₂O (10 mL) and the pale yellow solid was collected by filtration, to provide compound 221 (193 mg, 80%). Present as a mixture of E/Z isomers. Major isomer: ¹H NMR (400 MHz, Methanol-d₄) δ 7.68 (d, J=1.7 Hz, 1H), 7.64 (d, J=2.0 Hz, 1H), 7.50 (d, J=8.5 Hz, 1H), 7.46 (d, J=8.6 Hz, 1H), 7.32 (dd, J=8.6, 1.7 Hz, 1H), 7.20 (dd, J=8.6, 2.1 Hz, 1H), 6.73 (s, 1H), 4.84-4.77 (m, 2H), 3.74-3.66 (m, 2H), 3.18 (s, 9H). Mass calculated for (C₂₃H₂₂BrClF₃N₄O−I)⁺541.1, found 541.0.

### Synthesis of Compound 222

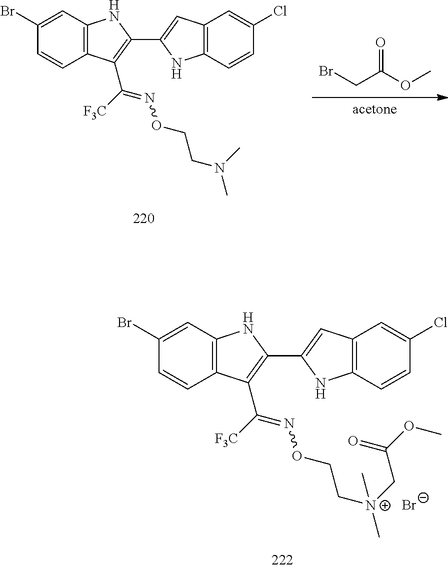

### Compound 222: N-(2-(((1-(6-Bromo-5′-chloro-1H,1′H-[2,2′-biindol]-3-yl)-2,2,2-trifluoroethylidene)amino)oxy)ethyl)-2-methoxy-N,N-dimethyl-2-oxoethan-1-aminium Bromide

A mixture of compound 220 (30 mg, 57 μmol) and methyl 2-bromoacetate (8 μL, 85 μmol) in acetone (2 mL) was stirred at rt for 20 h and then concentrated. The crude product was purified by silica gel chromatography, eluting with MeOH/DCM gradient, to provide the desired adduct 222 (24 mg, 62%). Present as a mixture of E/Z isomers. Major isomer: ¹H NMR (400 MHz, Methanol-d₄) δ 7.68 (d, J=1.7 Hz, 1H), 7.63 (d, J=2.1 Hz, 1H), 7.54 (d, J=8.6 Hz, 1H), 7.46 (d, J=8.9 Hz, 1H), 7.32 (dd, J=8.6, 1.8 Hz, 1H), 7.20 (dd, J=8.6, 2.0 Hz, 1H), 6.75 (bs, 1H), 4.87-4.83 (m, 2H), 4.66 (s, 2H), 4.08-4.02 (m, 2H), 3.85 (s, 3H), 3.37 (s, 6H). Mass calculated for (C₂₅H₂₄BrClF₃N₄O₃−Br)⁺599.1, found 599.0.

### Synthesis of Compound 223

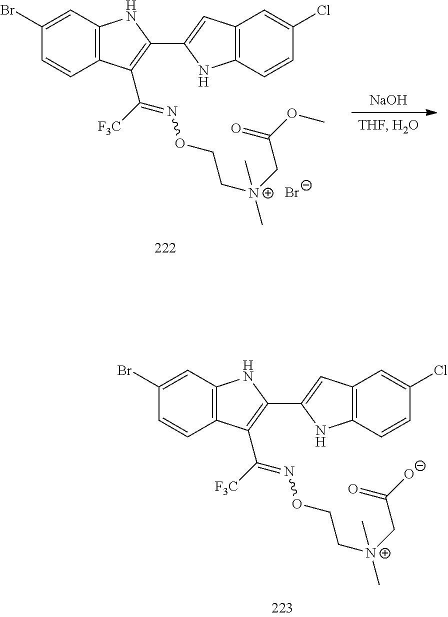

### Compound 223: 2-((2-(((1-(6-Bromo-5′-chloro-1H,1′H-[2,2′-biindol]-3-yl)-2,2,2-trifluoroethylidene)amino)oxy)ethyl)dimethylammonio)acetate

2M aqueous NaOH solution was added to a stirred solution of compound 222 (12 mg, 18 μmol) in THF (1 mL) until the solution was basic (˜pH 12) and the mixture was stirred at rt for 16 h. The reaction mixture was purified by preparative HPLC (ACN/H₂O with 0.1% TFA) to provide compound 223 (1.7 mg, 17%). ¹H NMR (400 MHz, Methanol-d₄) δ 7.68 (d, J=1.8 Hz, 1H), 7.63 (d, J=2.1 Hz, 1H), 7.48 (d, J=8.7 Hz, 1H), 7.38 (d, J=8.6 Hz, 1H), 7.30 (dd, J=8.6, 1.7 Hz, 1H), 7.20 (dd, J=8.7, 2.1 Hz, 1H), 6.76 (bs, 1H), 4.72-4.64 (m, 2H), 3.93 (s, 2H), 3.88-3.80 (m, 2H), 2.97 (s, 6H). Mass calculated for (C₂₄H₂₁BrClF₃N₄O₃+H)⁺585.0, found 584.9.

### Compound 224: 1-(6-bromo-5′-chloro-1H,1′H-[2,2′-biindole]-3-carbonyl)-4-(tert-butoxycarbonyl)piperazine-2-carboxylic Acid

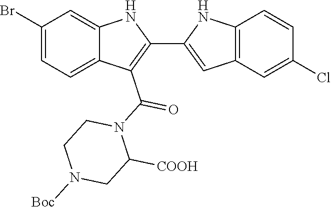 [SMILES: CC(C)(C)OC(=O)N1CCN(C(=O)c2c(-c3cc4cc(Cl)ccc4[nH]3)[nH]c3cc(Br)ccc23)C(C(=O)O)C1]

Prepared according to general method XXXIV from intermediate 72-iii and 4-Boc-piperazine-2-carboxylic acid with purification by concentration, then direct flash purification with a gradient of 50-100% EtOAc/Hexanes to afford the product as yellow solid (14 mg, 18%). ¹H NMR (400 MHz, DMSO) δ 12.21 (s, 1H), 7.64 (s, 2H), 7.52 (d, J=8.6 Hz, 2H), 7.26 (d, J=7.9 Hz, 1H), 7.20-6.85 (m, 2H), 4.69-4.35 (m, 1H), 3.85-3.36 (m, 6H), 1.36 (s, 9H). Mass calculated for (C₂₇H₂₆BrClN₄O₅−H)⁻ 599.1, found 599.0.

### Compound 225: 1-(6-bromo-5′-chloro-1H,1′H-[2,2′-biindole]-3-carbonyl)piperazine-2-carboxylic Acid

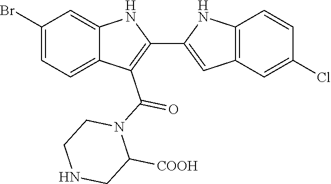 [SMILES: O=C(O)C1CNCCN1C(=O)c1c(-c2cc3cc(Cl)ccc3[nH]2)[nH]c2cc(Br)ccc12]

Prepared according to general method XXXIII from compound 224 to yield the title compound as a light brown solid (10 mg, quant.). ¹H NMR (400 MHz, DMSO) δ 12.21 (s, 1H), 7.64 (s, 2H), 7.52 (d, J=8.6 Hz, 2H), 7.26 (d, J=7.9 Hz, 1H), 7.20-6.85 (m, 2H), 5.44-4.34 (m, 2H), 3.85-3.35 (m, 5H), 1.36 (s, 9H). Mass calculated for (C₂₂H₁₈BrClN₄O₃+H)⁺501.0, found 500.8.

### Synthesis of Compound 226

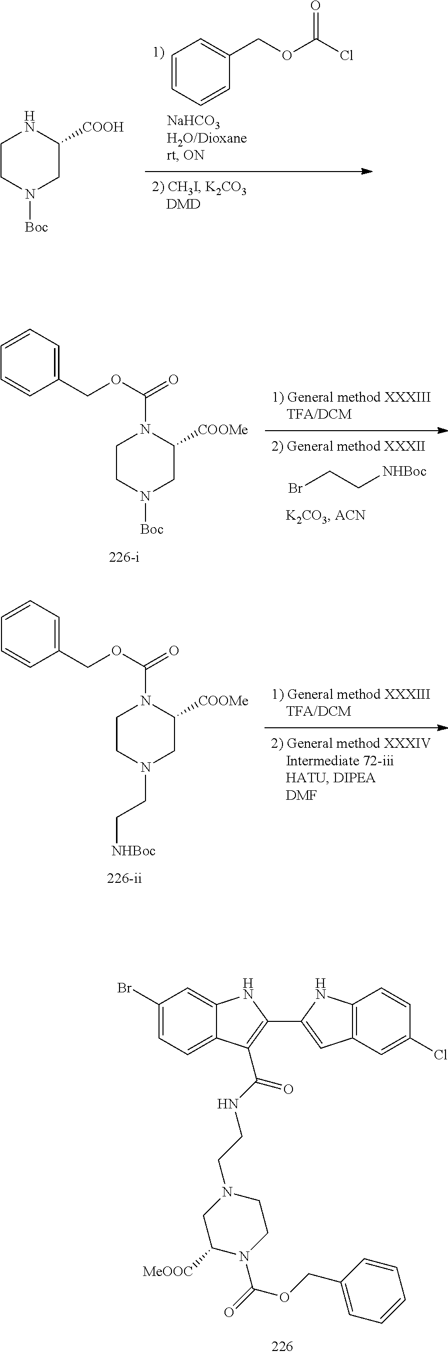 [SMILES: O=C(Cl)OCc1ccccc1 ; CC(C)(C)OC(=O)N1CCN[C@H](C(=O)O)C1 ; COC(=O)[C@@H]1CN(C(=O)OC(C)(C)C)CCN1C(=O)OCc1ccccc1 ; CC(C)(C)OC(=O)NCCBr ; COC(=O)[C@@H]1CN(CCNC(=O)OC(C)(C)C)CCN1C(=O)OCc1ccccc1 ; COC(=O)[C@@H]1CN(CCNC(=O)c2c(-c3cc4cc(Cl)ccc4[nH]3)[nH]c3cc(Br)ccc23)CCN1C(=O)OCc1ccccc1]

### Intermediate 226-i: 1-benzyl 4-(tert-butyl) 2-methyl (S)-piperazine-1,2,4-tricarboxylate

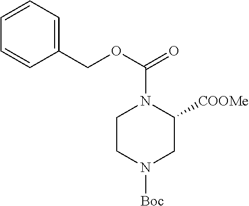 [SMILES: COC(=O)[C@@H]1CN(C(=O)OC(C)(C)C)CCN1C(=O)OCc1ccccc1]

To a stirred suspension of (S)-4-N-Boc-piperazine-2-carboxylic acid (502 mg, 2.18 mmol) in water (2.5 mL) was added NaHCO₃ (366.2 mg, 4.36 mmol), and the resulting suspension was stirred at ambient temperature for 30 minutes. A solution of benzyl chloroformate (744 mg, 4.36 mmol) in dioxane (4 mL) was then added and the reaction mixture was stirred at ambient temperature overnight. The reaction mixture was then diluted with water (5 mL) and extracted with EtOAc (2×15 mL). The combined organic layer was washed brine, dried over anhydrous Na₂SO₄, filtered and concentrated under reduced pressure. The remaining residue was then dissolved in DMF (7 mL) and K₂CO₃ (904 mg, 6.54 mmol) was added. After stirring for 5 minutes, CH₃I (928 mg, 6.54 mmol) was added slowly and the resulting mixture was stirred at ambient temperature for 2 h. The reaction was quenched with H₂O and extracted with EtOAc (2×15 mL). The combined organic layer was washed with brine, dried over anhydrous Na₂SO₄, filtered and concentrated under reduced pressure to leave brown oil as the product (800 mg, quant.). ¹H NMR (400 MHz, CDCl₃) δ 7.42-7.31 (m, 5H), 5.22-5.09 (m, 2H), 4.84-4.63 (m, 1H), 4.63-4.47 (m, 1H), 4.11-3.81 (m, 2H), 3.72 (d, J=22.6 Hz, 3H), 3.32 (s, 1H), 3.08 (d, J=13.5 Hz, 1H), 1.44 (s, 9H). Mass calculated for (C₁₉H₂₆N₂O₆+H)⁺379.2, found 379.1.

### Intermediate 226-ii: 1-benzyl 2-methyl (S)-4-(2-((tert-butoxycarbonyl)amino)ethyl)piperazine-1,2-dicarboxylate

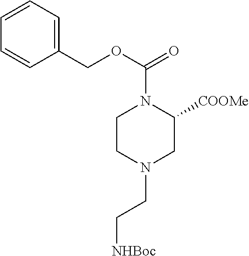 [SMILES: COC(=O)[C@@H]1CN(CCNC(=O)OC(C)(C)C)CCN1C(=O)OCc1ccccc1]

Prepared in two sequential steps: 1) according to general method XXXIII from intermediate 226-i to yield light brown oil residue; 2) according to general method XXXII from the light brown residue obtained from the first step and 2-(N-Boc-amino)ethyl bromide followed by filtration through celite, concentration and purification by silica gel column chromatography eluting with 12-100% EtOAc/Hexanes to afford the intermediate 226-ii as yellow oil (553 mg, 62%). ¹H NMR (400 MHz, CDCl₃) δ 7.44-7.29 (m, 5H), 5.17 (dd, J=14.4, 11.7 Hz, 2H), 4.82 (t, J=33.8 Hz, 2H), 4.04-3.84 (m, 1H), 3.77 (d, J=18.0 Hz, 3H), 3.49-3.10 (m, 4H), 2.76 (dd, J=28.8, 11.0 Hz, 1H), 2.60-2.46 (m, 1H), 2.47-2.32 (m, 1H), 2.31-2.08 (m, 2H), 1.46 (s, 9H). Mass calculated for (C₂₁H₃₁N₃O₆+H)⁺422.2, found 422.1.

### Compound 226: 1-benzyl 2-methyl (S)-4-(2-(6-bromo-5′-chloro-1H,1′H-[2,2′-biindole]-3-carboxamido)ethyl)piperazine-1,2-dicarboxylate

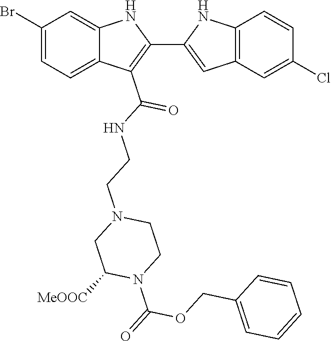 [SMILES: COC(=O)[C@@H]1CN(CCNC(=O)c2c(-c3cc4cc(Cl)ccc4[nH]3)[nH]c3cc(Br)ccc23)CCN1C(=O)OCc1ccccc1]

Prepared in two sequential steps: 1) according to general method XXXIII from intermediate 226-ii to yield brown oil residue; 2) according to general method XXXIV from intermediate 72-iii and the brown oil residue obtained from the first step followed by purification by silica gel column chromatography with 5-100% EtOAc/Hexanes to afford the product as yellow solid (205 mg, 58%). ¹H NMR (400 MHz, CDCl₃) δ 12.61 (s, 1H), 8.80 (s, 1H), 7.65-7.54 (m, 3H), 7.45-7.30 (m, 7H), 7.18 (dd, J=8.6, 1.8 Hz, 1H), 6.92-6.84 (m, 1H), 6.81 (s, 1H), 5.28-5.11 (m, 2H), 4.81 (d, J=53.8 Hz, 1H), 4.09-3.94 (m, 1H), 3.92-3.79 (m, 1H), 3.65-3.37 (m, 3H), 3.38-3.19 (m, 4H), 3.01-2.85 (m, 1H), 2.81-2.63 (m, 1H), 2.39-2.23 (m, 2H). Mass calculated for (C₃₃H₃₁BrClN₅O₅+H)⁺692.1, found 692.0.

### Compound 227: Methyl (S)-4-(2-(6-bromo-5′-chloro-1H,1′H-[2,2′-biindole]-3-carboxamido)ethyl)piperazine-2-carboxylate

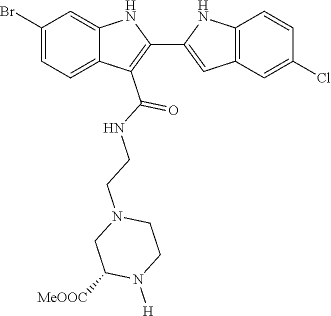 [SMILES: COC(=O)[C@@H]1CN(CCNC(=O)c2c(-c3cc4cc(Cl)ccc4[nH]3)[nH]c3cc(Br)ccc23)CCN1]

Compound 226 (100 mg, 0.144 mmol) was dissolved in TFA (3 mL, 17.7 mmol) and stirred under N₂ atmosphere at 70° C. for 3 h. It was then concentrated in vacuo and the remaining residue was purified by silica gel column chromatography with 50-100% (5% Et₃N/EtOAc)/Hexanes to afford the product (free base) as sticky orange solid (60 mg, 74%). ¹H NMR (400 MHz, CDCl₃) δ 12.65 (s, 1H), 9.03 (d, J=44.6 Hz, 1H), 7.68 (d, J=8.6 Hz, 1H), 7.58 (d, J=7.8 Hz, 2H), 7.41 (d, J=8.7 Hz, 1H), 7.34 (d, J=8.6 Hz, 1H), 7.17 (dd, J=8.7, 1.8 Hz, 1H), 7.05-6.96 (m, 1H), 6.82 (s, 1H), 3.83-3.70 (m, 1H), 3.67-3.57 (m, 2H), 3.53 (s, 3H), 3.36-3.25 (m, 1H), 3.12 (ddd, J=9.0, 5.6, 2.7 Hz, 1H), 2.96-2.81 (m, 2H), 2.80-2.58 (m, 4H), 2.57-2.39 (m, 1H). Mass calculated for (C₂₅H₂₅BrClN₅O₃+H)⁺ 558.1, found 558.0.

### Compound 228: (S)-4-(2-(6-bromo-5′-chloro-1H,1′H-[2,2′-biindole]-3-carboxamido)ethyl)-2-(methoxycarbonyl)piperazin-1-ium 2,2,2-trifluoroacetate

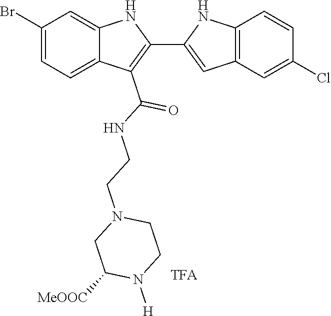 [SMILES: COC(=O)[C@@H]1CN(CCNC(=O)c2c(-c3cc4cc(Cl)ccc4[nH]3)[nH]c3cc(Br)ccc23)CCN1]

Compound 227 (10 mg, 0.018 mmol) was dissolve in TFA (500 μL) and stirred at ambient temperature for 30 minutes. The solution was then concentrated in vacuo and co-evaporated with PhMe (2×5 mL). The remaining residue was rinsed with Et₂O and was dried in vacuo to leave the product (TFA salt) as creamy white solid (10 mg, 82%). ¹H NMR (400 MHz, DMSO) δ 12.49 (s, 1H), 12.29 (s, 1H), 9.23 (s, 2H), 8.13 (t, J=4.9 Hz, 1H), 7.78 (d, J=8.6 Hz, 1H), 7.70 (d, J=1.7 Hz, 1H), 7.59 (dd, J=15.8, 5.1 Hz, 2H), 7.32 (dd, J=8.6, 1.6 Hz, 1H), 7.17-7.08 (m, 2H), 4.28 (s, 1H), 3.77-3.69 (m, 1H), 3.68 (s, 3H), 3.54-3.46 (m, 2H), 3.11-2.99 (m, 2H), 2.86-2.76 (m, 1H), 2.72-2.55 (m, 4H). Mass calculated for (C₂₇H₂₆BrClF₃N₅O₅−TFA+H)⁺ 558.1, found 558.0.

### Compound 229: (S)-4-(2-(6-bromo-5′-chloro-1H,1′H-[2,2′-biindole]-3-carboxamido)ethyl)piperazine-2-carboxylic Acid

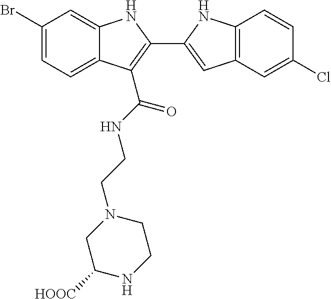 [SMILES: O=C(NCCN1CCN[C@H](C(=O)O)C1)c1c(-c2cc3cc(Cl)ccc3[nH]2)[nH]c2cc(Br)ccc12]

To a solution of Compound 227 (35 mg, 0.063 mmol) in THF/H₂O (2/1, 5 mL) was added LiOH (16 mg, 0.63 mmol), and the resulting solution was stirred at ambient temperature for 1 h. The reaction mixture was then quenched with 2M HCl and extracted with EtOAc (2×10 mL). The combined organic layer was washed with brine, dried over anhydrous Na₂SO₄, filtered and concentrated under reduced pressure to leave the product as white solid (30 mg, 87%). ¹H NMR (400 MHz, DMSO) δ 12.55 (s, 1H), 12.39 (s, 1H), 8.18 (t, J=5.2 Hz, 1H), 7.87 (d, J=8.6 Hz, 1H), 7.71 (s, 1H), 7.66-7.56 (m, 2H), 7.33 (dd, J=8.6, 1.4 Hz, 1H), 7.18-7.11 (m, 2H), 3.59-3.49 (m, 4H), 3.13 (d, J=12.8 Hz, 2H), 2.90-2.81 (m, 1H), 2.70-2.58 (m, 2H), 2.42-2.13 (m, 2H). Mass calculated for (C₂₄H₂₃BrClN₅O₃+H)⁺544.1, found 543.9.

### Compound Testing

The IC50 of selected compounds was determined according the following procedures described herein. The results are presented in Table 3 below. Further, the antimicrobial activity of selected compounds against the gram negative bacteria Klebsiella pneumoniae and Acinetobacter baumannii, as well as additional gram positive bacteria, including drug-resistant strains, was tested according to the procedures described herein The results are presented in Table 4 below.

### Bacterial Strains

Epidemic methicillin resistant S. aureus (MRSA) strain sequenced at the Sanger Centre (MRSA252, NRS71) was obtained from NARSA (Network on Antimicrobial Resistance in S. aureus). Methicillin sensitive S. aureus (ATCC 29213 and 25923), methicillin resistant S. aureus (ATCC BAA-1762), and Salmonella typhimurium (ATCC BAA-185) were obtained from ATCC, The Global Bioresource Center. Acinetobacter baumannii (ATCC 19606 and ATCC 17978), Klebsiella pneumonia (C238), vancomycin resistant Enterococci #2 (2010A) (VRE#2), MRSA (USA400, MW2) and Pseudomonas aeruginosa (PA0-1) were obtained from the laboratory of Dr B. B. Finlay at the University of British Columbia (Vancouver, Canada).

### Generation of Pyruvate Kinase (PK) Constructs

Genomic DNA of MRSA strain Sanger 252 extracted using DNeasy Tissue Kit™ (Qiagen™) was used as a template to generate the His-tagged MRSA PK. Human cDNA from MCF-7 breast cancer cell line (courtesy of Dr. J Wong, BC Cancer Research Center (Vancouver, Canada)) was used as a template to generate the full-length human M2 PK enzyme. The following primer sets were used creating appropriate restriction sites (NdeI and XhoI sites underlined): For cloning of MRSA PK: M27F 5′-CTACATATGAGAAAAACTAAAATTGTATG-3′ and M27R 5′-GTTCTCGAGTTATAGTACGTTTGCATATCCTTC-3′, for cloning of human M2 PK isoform: hM2F 5′-GATCATATGATGTCGAAGCCCCATAGTGAAGCC-3′ and hM2R 5′-GTTCTCGAGTCACGGCACAGGAACAACACGCATG-3′. The resulting PCR fragments for each construct were cloned into the NdeI and XhoI unique sites of the bacterial expression vector pET-28a (+) (Novagen™). This step resulted in plasmids pET-28a-MRSA and pET-28M2, which generated N-terminally His-tagged recombinant MRSA and human M2 PKs. The sequence and the correct reading frame of all constructs were verified by sequencing. Human M1, R and L PK constructs in pET-28-a(+) vectors (courtesy of Dr. L. Cantley, Harvard Medical, School (Boston, USA)) were used to generate relevant recombinant His-tagged human PK isoforms.

### Expression and Purification of Recombinant His-Tagged MRSA and Human PKs

MRSA and human constructs in pET-28a(+) were used to express relevant recombinant PK proteins in E. coli BL-21 (DE3). The proteins were expressed and purified using Ni-NTA agarose (Qiagen™) according to the manufacturer's protocol. Briefly, cells were grown to an absorbance of 0.4-0.5 at 600 nm in 2×YT medium, then induced with 0.1 mM IPTG for 3 h at 20° C. Cells were lysed by sonication on ice (3×10-s bursts with a 30-s recovery between bursts) in lysis buffer (0.2 mg/ml lysozyme, 50 mM Tris pH 7.5, 10 mM MgCl₂, 200 mM NaCl, 100 mM KCl, 10% glycerol, 10 mM imidazole, 0.5% NP-40 and 1 mM DTT containing Complete™ protease inhibitor). Cell lysates were cleared by centrifugation (18,000×g in a Beckman™ JA-20 rotor) for 20 min at 4° C. and PK isoforms were purified by batch binding to Ni-NTA resin. The resins were then packed in columns (1×2 cm) and washed with 400 column volumes lysis buffer containing 30 mM imidazole. His-tagged PK isoforms were eluted with the same buffer containing 300 mM imidazole. The proteins were dialyzed overnight at 4° C. against 2000 volumes of ice-cold 30 mM Tris pH 7.5, 25 mM KCl, 5 mM MgCl₂, 10% glycerol and 1 mM DTT to remove imidazole. All purification steps were done at 4° C.; enzymes were flash-frozen and stored at −70° C. Enzymatic activity of frozen protein preparations was stable for at least 10 months and up to 5 freeze/thaw cycles. Purity and physical integrity of proteins were assessed using SDS-polyacrylamide gel electrophoresis (SDS-PAGE) followed by coomassie blue staining. Protein concentration was estimated by Bradford assay (Bio-Rad Protein Assay™) using bovine serum albumin as a standard.

### Measurement of PK Activity

Candidate MRSA PK inhibitors were assayed for their ability to inhibit enzymatic activities of MRSA and human PKs. PK activity was determined using a continuous assay coupled to lactate dehydrogenase (LDH) in which the change in absorbance at 340 nm owing to oxidation of NADH was measured using a Benchmark Plus™ microplate spectrophotometer (Bio-Rad Laboratories, Hercules, Calif.). The reaction contained 60 mM Na⁺-HEPES, pH 7.5, 5% glycerol, 67 mM KCl, 6.7 mM MgCl₂, 0.24 mM NADH, 5.5 units L-LDH from rabbit muscle (Sigma-Aldrich, St. Louis, Mo.), 2 mM ADP and 10 mM PEP (i.e. close to the Kₘ of MRSA PK, so that the IC₅₀ values should approximate the Kᵢ) in a total volume of 200 μl. Reactions were initiated by the addition of 15 nM of one of the PK enzymes. PK activity proportional to the rate of change at 340 nm was expressed as specific activity (μmol/min/mg), which is defined as the amount of PK that catalyzes the formation of one micromole of either product per minute. Inhibitors were dissolved in DMSO with the final concentration of the solvent never exceeding 1% of the assay volume. IC₅₀ values were calculated by curve fitting on a four-parameter dose-response model with variable slope using Graphpad Prism 5.0™ (GraphPad™ Software Inc., La Jolla, Calif.). In all studies, less than 10% of total PEP was exhausted during the reaction. Reactions were performed at 30° C. for up to 5 min. All values determined represent at least two measurements, in triplicate (Tables 1-6) or duplicate unless mentioned otherwise. Mode-of-inhibition and Kᵢ values were determined by simultaneously changing the inhibitor concentration (0-400 nM) and substrate PEP concentration (2-20 mM) while keeping the level of the ADP substrate fixed at 2 mM. The resulting curve at each inhibitor concentration was fitted by nonlinear regression to the allosteric sigmoidal kinetic model using Graphpad Prism™. Kᵢ values were obtained by nonlinear regression curve-fitting using the following equation:

Apparent Vₘₐₓ=Vₘₐₓ/(1+[I]/Kᵢ) (1)

### In Vitro Susceptibility Testing

The antimicrobial activities of PK inhibitor candidates were determined using the 96-well microtiter standard 2-fold serial broth microdilution method as described by CLSI (formerly NCCLS) with the various gram-positive and gram-negative bacteria species mentioned above. Bacteria from a single colony were grown, overnight in either BHI Broth (VRE), mueller hinton broth (S. aureus 29213; MRSA USA400, A. baumannii 19606, MRSA BAA-1762) or L-broth (P. aeruginosa, S. typhimurium, K. pneumonia and A. baumannii 17978). Each compound was prepared in DMSO with 2-fold serial dilutions to give a final concentration of, 0.031 to 64 μg/ml. 10 μl of the compound solution was then added, in duplicate, to either, 190 μl of cation adjusted mueller hinton broth (CAMHB) or 190 μl CAMHB containing ˜2.5×10⁵ CFU/ml of bacteria (final compound concentration 0.031 to 64 μg/ml). Culture plates were incubated for 18-24 h at 37° C., and optical density at 600 nm (OD₆₀₀) was measured using a Benchmark Plus™ microplate spectrophotometer (Bio-Rad™). The absorbance control values for the series containing CAMHB and inhibitor were subtracted as background from the corresponding infected wells. Minimal inhibitory concentration (MIC) was defined as the lowest concentration of test compound leading to complete inhibition of cell growth in relation to compound-free control wells as determined by optical density. Vancomycin, methicillin and ciprofloxicin were used as reference compounds. All assays were run in triplicate or duplicate. Experiments were replicated at least twice to verify reproducibility using the above conditions.

Determination of Mammalian Cytotoxicity

The cytotoxic activities of compounds were determined for HeLa cells 229 (ATCC:CCL-2-.1) in microtiter cultures by measuring dehydrogenase activity using CellTiter 96® AQᵤₑₒᵤₛ One Solution Cell Proliferation Assay™ (Promega™, Madison, Wis., USA), according to the manufacturer's protocol. Freshly split cells were seeded into microtiter wells (2×10⁴/well) and grown for 24 hours. The original media was then removed and replaced with media containing the desired concentration of compound or solvent control (i.e., DMSO). Plates were incubated for 24 h at 37° C. in a humidified incubator with a 5% CO₂ atmosphere. At the end of the growth period, cells were lysed by the addition of 20 μl of Cell Titer 96 Aqueous One™ solution, and the incubation was continued for another 3 h at 37° C. Production of formazan was determined at 490 nm on Benchmark Plus™ microplate spectrophotometer (Bio-Rad™). To control for intrinsic absorbance, control series containing inhibitor dilutions but no cells were run for every experiment and the resulting absorbance values were subtracted as background from the experimental readings. Growth in compound-free control wells was considered as 100% and percentage of growth inhibition was calculated for each compound concentration. Cytotoxicity was quantified as the CC₅₀, the concentration of compound that inhibited 50% of conversion of MTS to formazan. The “selectivity index” is defined as the ratio of the mammalian cell cytotoxicity to the MIC against S. aureus (i.e., CC₅₀/MIC). Positive control measurements were performed with xanthohumol (HeLa cells: CC₅₀≈9 μg/ml). All assays were performed three times in triplicate.

TABLE 3
MRSA Pyk Inhibitory Activity

| | IC₅₀ (nM) |
|---|---|
| Compound No. | [a - <100 nM, b 100 nM to 1000 nM, c - >1000 nM] |
| 1 | a |
| 2 | a |
| 3 | a |
| 4 | a |
| 5 | a |
| 6 | b |
| 7 | b |
| 8 | b |
| 9 | b |
| 10 | b |
| 12 | b |
| 13 | b |
| 14 | b |
| 16 | a |
| 17 | b |
| 18 | a |
| 19 | b |
| 21 | a |
| 22 | a |
| 23 | a |
| 24 | b |
| 25 | b |
| 26 | b |
| 27 | a |
| 28 | b |
| 29 | c |
| 30 | b |
| 31 | a |
| 32 | a |
| 33 | c |
| 34 | c |
| 35 | b |
| 36 | b |
| 37 | b |
| 38 | a |
| 39 | a |
| 40 | b |
| 41 | a |
| 42 | b |
| 43 | a |
| 44 | b |
| 45 | a |
| 46 | c |
| 47 | c |
| 49 | c |
| 50 | c |
| 51 | c |
| 52 | a |
| 53 | a |
| 54 | a |
| 55 | c |
| 57 | b |
| 58 | a |
| 59 | b |
| 60 | a |
| 61 | b |
| 62 | b |
| 63 | b |
| 64 | a |
| 65 | a |
| 66 | b |
| 67 | c |
| 68 | a |
| 69 | a |
| 70 | c |
| 71 | c |
| 72 | a |
| 73 | a |
| 74 | a |
| 75 | c |
| 76 | c |
| 77 | a |
| 78 | a |
| 79 | a |
| 80 | a |
| 81 | a |
| 82 | a |
| 83 | c |
| 85 | b |
| 86 | a |
| 87 | a |
| 88 | b |
| 89 | c |
| 90 | b |
| 92 | a |
| 93 | a |
| 94 | b |
| 95 | b |
| 96 | c |
| 97 | c |
| 98 | b |
| 102 | a |
| 103 | a |
| 104 | a |
| 105 | a |
| 106 | a |
| 107 | a |
| 108 | c |
| 112 | b |
| 113 | a |
| 114 | b |
| 116 | a |
| 117 | a |
| 118 | a |
| 119 | a |
| 120 | a |
| 121 | a |
| 122 | a |
| 123 | a |
| 124 | a |
| 125 | a |
| 126 | a |
| 127 | a |
| 129 | a |
| 130 | a |
| 133 | a |
| 136 | a |
| 137 | a |
| 138 | a |
| 139 | a |
| 142 | a |
| 144 | a |
| 145 | a |
| 146 | a |
| 147 | a |
| 148 | a |
| 149 | a |
| 150 | a |
| 183 | a |
| 184 | a |
| 185 | a |
| 186 | a |
| 187 | a |
| 188 | a |
| 189 | a |
| 190 | a |
| 191 | a |
| 192 | a |
| 193 | a |
| 195 | a |
| 196 | a |
| 197 | a |
| 198 | a |
| 199 | a |
| 200 | a |
| 201 | a |
| 202 | a |
| 203 | a |
| 204 | a |
| 205 | a |
| 206 | a |
| 207 | a |
| 208 | c |
| 216 | b |
| 217 | a |
| 218 | a |
| 219 | a |
| 220 | a |
| 221 | a |
| 222 | a |
| 223 | b |
| 224 | a |
| 229 | a |

TABLE 4
Antimicrobial Activity against Gram Negative and Gram Postive Bacterial Strains

| | | | MRSA | | | | | |
|---|---|---|---|---|---|---|---|---|
| | S. aureus | MSRA | ATCC | | | | | |
| Compound | ATCC29213 | USA400 | BAA1762 | VRE#2 | K. pneumoniae | A. baumannii | P. aeruginosa | S. typhimurium |
| No. | (ug/mL) | (ug/mL) | (ug/mL) | (ug/mL) | (ug/mL) | (ug/mL) | (ug/mL) | (ug/mL) |
| 1 | >64 | | | | | | | |
| 2 | 2 | | | | | | | |
| 3 | 16 | | | | | | | |
| 4 | 8 | | | | | | | |
| 5 | 32 | | | | | | | |
| 6 | 8 | 8 | | 8 | 8 | 64* | | |
| 7 | 8 | 8 | | 8 | 8 | >64* | | |
| 8 | 64 | | | | | | | |
| 9 | 32 | | | | | | | |
| 10 | >64 | | | | | | | |
| 11 | 2 | 2 | | | 4 | >64* | | |
| 12 | 4 | 4 | | | 4 | 16 | | |
| 13 | 1 | | | | | | | |
| 14 | 1 | | | | | | | |
| 15 | 1 | | | | | | | |
| 16 | 1 | | | | | | | |
| 17 | 2 | | | | | | | |
| 18 | 1 | | | | | | | |
| 19 | 0.5 | | | | | | | |
| 20 | 64 | | | | | | | |
| 21 | >64 | | | | | | | |
| 22 | >64 | | | | | | | |
| 23 | 8 | | | | | | | |
| 24 | >64 | | | | | | | |
| 25 | 8 | | | | | | | |
| 26 | 8 | | | | | | | |
| 27 | >64 | | | | | | | |
| 28 | >64 | | | | | | | |
| 30 | 16 | | | | | | | |
| 31 | 16 | | | | | | | |
| 32 | >64 | | | | | | | |
| 33 | >64 | | | | | | | |
| 34 | 16 | | | | | | | |
| 35 | >64 | | | | | | | |
| 36 | >64 | | | | | | | |
| 38 | >64 | | | | | | | |
| 39 | >64 | | | | | | | |
| 40 | 8 | | | 8 | 16 | >64 | | |
| 41 | >64 | >64 | | >64 | >64 | | | |
| 42 | >64 | | | | | | | |
| 43 | 32 | 32 | | 16 | 32 | | | |
| 44 | 32 | 32 | | 16 | 32 | | | |
| 45 | >64 | | | | 32 | | | |
| 46 | 1 | | | | | | | |
| 47 | >64 | | | | | | | |
| 48 | >64 | | | | >64 | | | |
| 49 | >64 | | | | | | | |
| 50 | >64 | | | | | | | |
| 51 | >64 | | | | | | | |
| 52 | 16 | | | | | | | |
| 53 | 8 | | | | | | | |
| 54 | >64 | | | | | | | |
| 55 | >64 | | | | | | | |
| 56 | >64 | | | | | | | |
| 57 | >64 | | | >64 | | | | |
| 58 | >64 | | | | | | | |
| 59 | >64 | | | | | | | |
| 60 | 4 | | | | | >64 | | |
| 61 | 4 | 4 | | 4 | 4 | >64* | | |
| 62 | >64 | | | | | | | |
| 63 | >64 | | | | | | | |
| 64 | >64 | | | | | | | |
| 65 | 1 | | | | | | | |
| 66 | 2 | | | | | | | |
| 67 | >64 | | | | | | | |
| 68 | 8 | | | | | | | |
| 69 | 16 | | | | | | | |
| 70 | >64 | | | | | | | |
| 72 | 8 | | | 8 | 8 | >64* | | |
| 73 | 8 | | | 8 | 8 | 64 | | |
| 74 | 32 | | | 64 | | | | |
| 75 | >64 | | | | | | | |
| 76 | >64 | | | | | | | |
| 77 | 4 | 4 | | | 4 | | | |
| 78 | >64 | | | | | | | |
| 79 | >64 | | | | | | | |
| 80 | >64 | | | | | | | |
| 81 | >64 | | | | | | | |
| 82 | >64 | | | | | | | |
| 84 | 64 | >64 | | | 16 | | | |
| 85 | >64 | | | | | | | |
| 86 | 64 | 64 | | 64 | >64 | | | |
| 87 | >64 | >64 | | >64 | >64 | | | |
| 88 | >64 | | | | >64 | | | |
| 89 | >64 | | | | >64 | | | |
| 90 | >64 | | | | >64 | | | |
| 91 | >64 | | | | >64 | | | |
| 92 | 64 | | | 32 | | | | |
| 93 | 32 | >64 | | 64 | >64 | | | |
| 94 | 32 | >64 | | >64 | >64 | | | |
| 95 | 32 | 64 | | 64 | 64 | | | |
| 96 | 64 | >64 | | >64 | >64 | | | |
| 98 | 32 | | | | >64 | | | |
| 99 | 1 | | | | 8 | | | |
| 100 | >64 | | | | >64 | | | |
| 101 | 8 | >64 | | | 32 | | | |
| 102 | 8 | 8 | | 16 | 16 | | | 8 |
| 103 | >64 | | | | >64 | | | |
| 104 | >64 | | | | >64 | | | |
| 105 | >64 | | | | >64 | | | |
| 106 | 8 | 8 | | 8 | 8 | >64 | | |
| 107 | 4 | 4 | | 4 | 4 | | | |
| 108 | 1 | 2 | | 2 | 2 | | | |
| 109 | >64 | | | | >64 | | | |
| 110 | >64 | | | | >64 | | | |
| 111 | >64 | | | | >64 | | | |
| 112 | 32 | | | >64 | >64 | >64* | | |
| 113 | >64 | | | | >64 | | | |
| 114 | 32 | 8 | | 4 | 8 | | | |
| 115 | >64 | | | | >64 | | | |
| 116 | >64 | | | | | | | |
| 117 | 8 | | 8 | 8 | | 32 | | |
| 118 | 16 | | 16 | 8 | | 32 | | |
| 119 | 16 | | 16 | 8 | | 64 | | |
| 120 | >64 | | | | | >64* | | |
| 121 | >64 | | | | | >64* | | |
| 122 | 8 | | 8 | 8 | | | | |
| 123 | 8 | | 8 | 8 | | | | |
| 124 | 8 | | 8 | 8 | | 16 | | |
| 125 | >64 | | | | | >64* | | |
| 126 | 16 | | 16 | 8 | | 32 | | |
| 127 | >64 | | | | | >64* | | |
| 128 | >64 | | | >64 | | >64 | | |
| 129 | 16 | | 16 | 16 | | | | |
| 130 | 16 | | 16 | 16 | | >64 | | |
| 131 | >64 | | | | | 64* | | |
| 132 | >64 | | | | | | | |
| 134 | >64 | | | | | >64* | | |
| 135 | >64 | | | | | >64* | | |
| 136 | 8 | | 8 | 8 | | 16 | | |
| 137 | 8 | | 8 | 8 | | 32 | | |
| 138 | 4 | | 8 | 4 | | 64 | | |
| 139 | 4 | | 8 | 8 | | | | |
| 140 | >64 | | | | >64 | | | |
| 141 | 0.5 | | 0.5 | | | | | |
| 142 | >64 | | | | | >64* | | |
| 143 | >64 | | | | | >64* | | |
| 144 | 8 | | 16 | 16 | | | | |
| 145 | 8 | | 16 | 16 | | >64* | | |
| 146 | 32 | | 32 | 64 | | >64* | | |
| 147 | 16 | | 16 | 16 | | >64* | | |
| 148 | 8 | | 16 | 16 | | >64 | | |
| 149 | 16 | | 16 | 16 | | >64 | | |
| 150 | 16 | | 16 | 32 | | >64 | | |
| 151 | >64 | | | | | | | |
| 152 | 1 | | 1 | 2 | | >64* | | |
| 153 | >64 | | | | | >64* | | |
| 154 | >64 | | | | | >64* | | |
| 155 | 0.5 | | 0.5 | 0.5 | | >64* | | |
| 156 | 0.5 | | 1 | 1 | | 64* | | |
| 157 | | | | | | >64* | | |
| 158 | 2 | | 1 | 2 | | >64* | | |
| 159 | 2 | | 1 | | | >64 | | |
| 160 | 4 | | 4 | 8 | | >64 | | |
| 161 | 16 | | 8 | 16 | | >64 | | |
| 162 | >64 | | | | | | | |
| 163 | >64 | | | | | | | |
| 164 | >64 | | | | | | | |
| 165 | >64 | | | | | | | |
| 166 | 1 | | 0.5 | 0.5 | | >64 | | |
| 167 | >64 | | | | | | | |
| 168 | 1 | | 1 | 16 | | >64 | | |
| 169 | 1 | | 0.5 | 2 | | >64 | | |
| 170 | 4 | | 2 | | | | | |
| 171 | >64 | | | | | | | |
| 172 | 0.5 | | 0.5 | 1 | | >64 | | |
| 173 | 16 | | 16 | 32 | | >64 | | |
| 174 | 1 | | 1 | 1 | | >64 | | |
| 175 | 32 | | 16 | 32 | | | | |
| 176 | 8 | | 8 | 16 | | >64 | | |
| 177 | >64 | | | | | | | |
| 178 | >64 | | | | | | | |
| 179 | >64 | | | | | | | |
| 180 | >64 | | | | | | | |
| 181 | >64 | | | | | >64* | | |
| 182 | 8 | | 0.5 | 64 | | | | |
| 183 | >64 | | | | | >64* | | |
| 184 | >64 | | 16 | >64 | | >64 | | |
| 185 | >64 | | | | | >64* | | |
| 186 | >64 | | | | | | | |
| 187 | 64 | | | | | | | |
| 188 | >64 | | | | | >64* | | |
| 189 | >64 | | | | | >64* | | |
| 190 | 8 | | 8 | 16 | | >64* | | |
| 191 | 16 | | 16 | 16 | | >64 | | |
| 192 | >64 | | | | | >64* | | |
| 193 | >64 | | | | | >64 | | |
| 194 | >64 | | | | | >64* | | |
| 195 | 64 | | 64 | | | >64* | | |
| 196 | 16 | | 8 | >64 | | >64 | | |
| 197 | 8 | | 8 | 8 | | >64 | | |
| 198 | >64 | | | | | | | |
| 199 | 4 | | 4 | 4 | | 8 | | 32 |
| 200 | 4 | | 1 | 1 | | >64 | | |
| 201 | 4 | | 4 | 4 | | 16 | 16 | 16 |
| 202 | 4 | | 4 | 4 | | >64* | | |
| 203 | 8 | | 8 | 16 | | | | |
| 204 | 64 | | | | | >64* | | |
| 205 | 32 | 32 | 64 | | | >64* | | |
| 206 | 8 | | 8 | 4 | | 16 | 32 | 64 |
| 207 | 0.5 | 1 | | 32 | 16 | >64 | | |
| 208 | 8 | | 8 | 16 | | >64* | | |
| 209 | >64 | | | | | >64* | | |
| 210 | >64 | | | | | >64* | | |
| 211 | >64 | | | | | >64* | | |
| 212 | >64 | | | | | >64* | | |
| 213 | >64 | | | | | >64* | | |
| 214 | >64 | | | | | >64* | | |
| 215 | >64 | | | | | >64 | | |
| 216 | 1 | | 1 | 1 | | 64 | | |
| 217 | 1 | | 1 | 1 | | >64 | | |
| 218 | 2 | | 2 | 2 | | >64 | | |
| 219 | 8 | | 8 | 4 | | >64 | | |
| 220 | 2 | | 2 | 2 | | >64 | | |
| 221 | 1 | | 2 | 2 | | 32 | 32 | 64 |
| 222 | 2 | | 2 | 4 | | 32 | | |
| 223 | 16 | | 8 | 8 | | >64 | | |
| 224 | 32 | | 32 | 32 | | | | |
| 225 | >64 | | | | | | | |
| 226 | >64 | | | | | | | |
| 227 | >64 | | | | | | | |
| 228 | >64 | | | | | | | |
| 229 | >64 | | | | | | | |

K. pneumoniae (C238);
A. baumannii (ATCC 19606);
VRE#2 (2010A);
MRSA MW2 (USA400);
S. aureus (ATCC29213);
*A. baumannii (ATCC 17978);
P. aeruginosa (PA01);
S. typhimurium (ATCC BAA-185);
MRSA (ATCC BAA-1762)

### In Vivo Antimicrobial Activity

The antimicrobial activity of compounds 23 and 60 against S. aureus ATCC 29123 was tested in vivo using a thigh infection model in neutropenic mice. Briefly, animals (female CD-1 mice, 5 weeks of age) were made neutropenic prior to S. aureus thigh infection by pre-treating with cyclophosphamide (150 mg/kg, IP, −4 and −1 days pre-inoculation). On the inoculation day (day 0), mice were infected with S. aureus at time zero (t=0). Animals were individually monitored for adverse reactions for 30 min post-infection.

Compounds 23 and 60 were prepared for IV administration and for oral administration. Vancomycin was administered as a solution in PBS. The test compounds were administered at 2 and 8 hours post-infection and animals were individually monitored for adverse reactions for 30 min after each injection. All animals were then monitored hourly from 20 hours post infection to the endpoint (t=24 hr post infection). At the indicated timepoint, animals were sacrificed and the injected thigh collected.

Quantitative enumeration of bacterial load was determined by plating serial dilutions from homogenized thigh muscles. The plates were incubated and colony counts were determined. CFU per mL calculated was calculated. A 3-log reduction in CFU was observed for compounds 23 and 60 upon dosing at 10 mg/kg IV BID.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. Furthermore, numeric ranges are provided so that the range of values is recited in addition to the individual values within the recited range being specifically recited in the absence of the range. The word “comprising” is used herein as an open-ended term, substantially equivalent to the phrase “including, but not limited to”, and the word “comprises” has a corresponding meaning. As used herein, the singular forms “a”, “an” and “the” include plural references unless the context clearly dictates otherwise. Thus, for example, reference to “a thing” includes more than one such thing. Citation of references herein is not an admission that such references are prior art to the present invention. Furthermore, material appearing in the background section of the specification is not an admission that such material is prior art to the invention. All citations are expressly incorporated herein in their entirety by reference. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

What is claimed is:

1. A compound having a structure of formula (1): 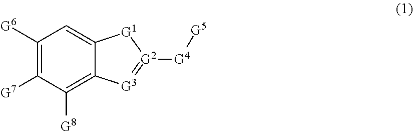
or a salt thereof,
wherein:
G¹ is NH, O, or S;
G², G³ and G⁴ may either: i) together form a ring moiety selected from the group consisting of: 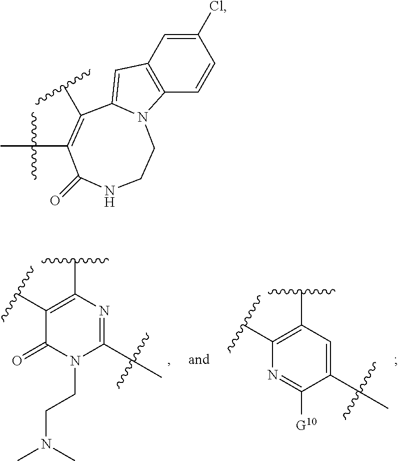
or
ii) together do not form a ring moiety wherein G² is C; G³ is N, CH or CG⁹; and
G⁴ is selected from the group consisting of: a bond, 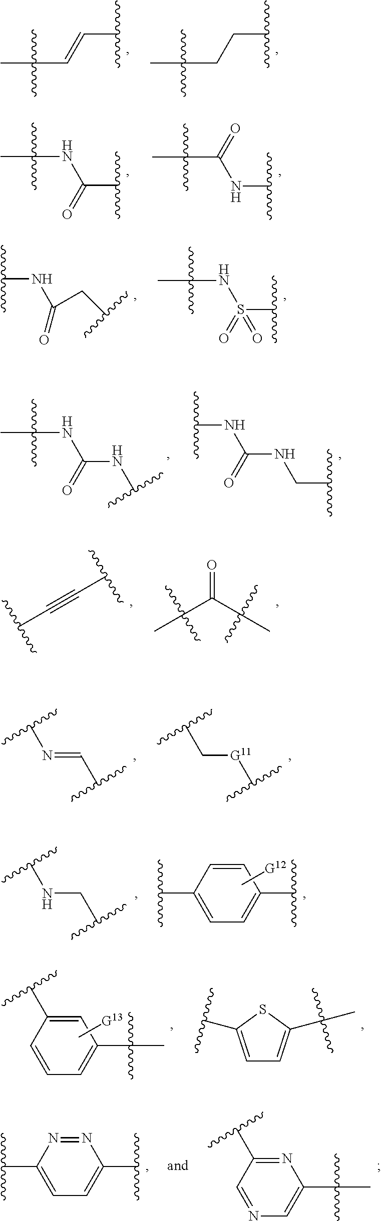
G⁵ is absent, 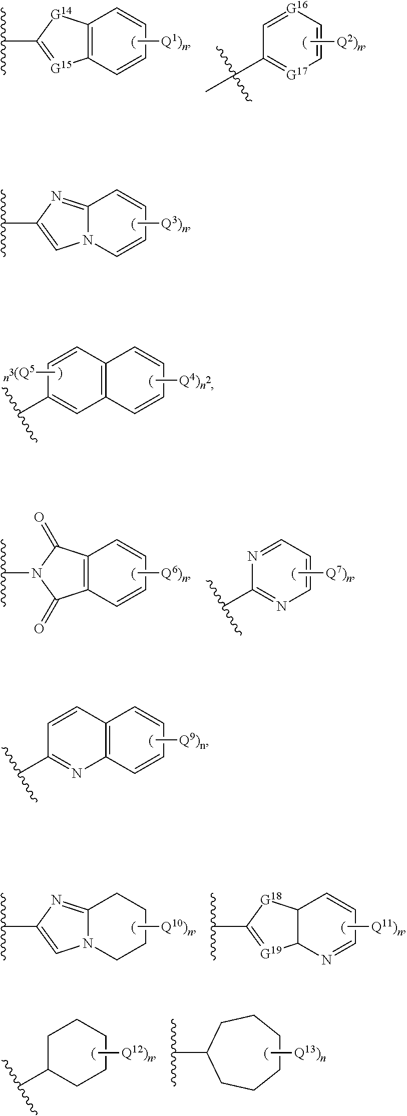
or a 5-membered heteroaryl optionally substituted with (Q⁸)ₙ and containing 1 or 2 heteroatoms each heteroatom independently selected from N, O and S;
G⁶ is H, halogen, CF₃, NO₂, substituted (C₁₋₁₁)alkyl, unsubstituted (C₁₋₁₁)alkyl, substituted (C₁₋₁₁)alkoxyl, unsubstituted (C₁₋₁₁) alkoxyl, substituted (C₆₋₁₁)aryloxy, unsubstituted (C₁₋₁₁)aryloxy, C(O)OR⁵⁰, or 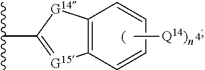
G⁷ is H, halogen, CF₃, NO₂, substituted (C₁₋₁₁)alkyl, unsubstituted (C₁₋₁₁)alkyl, substituted (C₁₋₁₁) alkoxyl, unsubstituted (C₁₋₁₁) alkoxy, substituted (C₆₋₁₁)aryloxy, unsubstituted (C₆₋₁₁)aryloxy, C(O)OR⁵¹, or 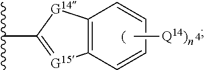
R⁵⁰ and R⁵¹ are each independently substituted (C₁₋₆)alkyl, unsubstituted (C₁₋₆)alkyl, substituted (C₁₋₆)heteroalkyl or unsubstituted (C₁₋₆) heteroalkyl;
G⁸ is H, C(═O)N(CH₃)₂, or C(═O)N(H)C(H₂)C₆H₅;
G⁹ is CF₃, —SO₂NH₂, —NH₂, —C(CF₃)₂OH, —C(CF₃)(H)OH, —C(CF₃)(CH₃)OH, —C(NOH)C(R²¹)(R²²)(R²³), C(NOH)N(R²⁴)(R²⁵), C(NOR⁶⁰)C(R⁶¹)(R⁶²)(R⁶³),
substituted (C₁₋₆) alkyl-NR⁶⁴R⁶⁵, unsubstituted (C₁₋₆) alkyl-NR⁶⁴R⁶⁵,
substituted (C₆₋₁₁) aryl, unsubstituted (C₁₀)aryl,
substituted (C₁₋₁₁) heteroaryl, unsubstituted (C₁₋₁₁) heteroaryl,
substituted (C₆₋₁₁) arylcarbonyl, unsubstituted (C₆₋₁₁) arylcarbonyl,
substituted (C₁₋₁₁) heteroarylcarbonyl, unsubstituted (C₁₋₁₁) heteroarylcarbonyl,
—CO-substituted-carbocycle, —CO-unsubstituted-carbocycle,
—CO-substituted-heterocarbocycle, —CO-unsubstituted-heterocarbocycle,
—CO-substituted-C(₁₋₆)alkyl-OR¹, —CO-unsubstituted-C(₁₋₆)alkyl-OR¹,
—CO-substituted-C(₁₋₆)alkyl-NR²R³, —CO-unsubstituted-C(₁₋₆)alkyl-NR²R³,
—CO-substituted-C(₁₋₆)alkyl-C(O)OR⁴, —CO-unsubstituted-C(₁₋₆)alkyl-C(O)OR⁴;
—CO-substituted-C(₁₋₆)alkyl-C(O)NR⁵R⁶, —CO-unsubstituted-C(₁₋₆)alkyl-C(O)NR⁵R⁶,
—C(O)NR⁷R⁸, —C(O)OR⁹, —C(O)C(O)OR¹², —C(O)C(O)NR¹³R¹⁴, —NR¹⁵R¹⁶,
—N(H)C(O)substituted-C(₁₋₆)alkyl, —N(H)C(O)unsubstituted-C(₁₋₆)alkyl,
—N(H)C(O)substituted-C(₁₋₆)haloalkyl, —N(H)C(O)unsubstituted-C(₁₋₆)haloalkyl,
—N(H)C(O)substituted-C(₆₋₁₁)aryl, —N(H)C(O)unsubstituted-C(₆₋₁₁)aryl,
—N(H)C(O)substituted-C(₁₋₁₁)heteroaryl, —N(H)C(O)unsubstituted-C(₁₋₁₁)heteroaryl,
—N(H)C(O)NR¹⁷R¹⁸,
—N(H)CO-substituted-C(₁₋₆)alkyl-OR¹⁹, —N(H)CO-unsubstituted-C(₁₋₆)alkyl-OR¹⁹,
each of R¹, R², R³, R⁴, R⁵, R⁶, R¹², R¹³, R¹⁴, R¹⁷, R¹⁸, R¹⁹, R²⁴, and R²⁵ is independently selected from the group consisting of: H, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl, and
each of R²¹, R²², R²³, R⁶¹, R⁶² and R⁶³ is independently selected from the group consisting of: H, F, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl;
each pair: a) R² and R³, b) R⁵ and R⁶, c) R¹³ and R¹⁴, and d) R¹⁷ and R¹⁸ may alternately be and independently as a pair be a 3-7 membered substituted heterocarbocyclic ring or a 3-7 membered unsubstituted heterocarbocyclic ring;
R⁶⁰ is unsubstituted C(₁₋₁₁)alkyl, substituted C(₁₋₁₁)alkyl, unsubstituted C(₁₋₁₁)alkyl-NR⁶⁶R⁶⁷, substituted C(₁₋₁₁)alkyl-NR⁶⁶R⁶⁷, unsubstituted C(₁₋₁₁)alkyl-N⁺R⁶⁸R⁶⁹R⁷⁰, or substituted C(₁₋₁₁)alkyl-N⁺R⁶⁸R⁶⁹R⁷⁰, wherein R⁶⁶ and R⁶⁷ are each independently H, unsubstituted C(₁₋₁₁)alkyl or substituted C(₁₋₁₁)alkyl, and R⁶⁸, R⁶⁹ and R⁷⁰ are each independently unsubstituted C(₁₋₁₁)alkyl, or substituted C(₁₋₁₁)alkyl,
each of R¹⁵ and R¹⁶ is independently selected from the group consisting of: H, substituted C(₁₋₆)alkyl, substituted C(₁₋₁₁)aryl, substituted C(₁₋₁₁)heteroaryl, substituted C(₇₋₁₁)aralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl, or R¹⁵ and R¹⁶ may alternately be a 3-7 membered unsubstituted heterocarbocyclic ring;
each of R⁶⁴ and R⁶⁵ is independently selected from the group consisting of: H, substituted C(₃₋₆)alkyl, substituted C(₁₋₁₁)aryl, substituted C(₁₋₁₁)heteroaryl, substituted C(₇₋₁₁)aralkyl, unsubstituted C(₂₋₁₁)alkyl, unsubstituted C(₁₋₁₁)aryl, unsubstituted C(₁₋₁₁)heteroaryl, and unsubstituted C(₈₋₁₁)aralky, or R⁶⁴ and R⁶⁵ may alternately be a 3-7 membered substituted heterocarbocyclic ring or a 3-7 membered unsubstituted heterocarbocyclic ring;
each of R⁷ and R⁸ are either
I) independently selected from the group consisting of: H, substituted C(₁₋₆)alkyl, substituted C(₁₋₆)alkyl-NR⁵²R⁵³, unsubstituted C(₁₋₆)alkyl-NR⁵²R⁵³, substituted C(₁₋₆)alkyl-N⁺R⁷¹R⁷²R⁷³, unsubstituted C(₁₋₆)alkyl-N⁺R⁷¹R⁷²R⁷³, substituted C(₁₋₆)alkyl-OC(O)unsubstituted C(₁₋₆)alkyl-NR⁷⁴R⁷⁵, unsubstituted C(₁₋₆)alkyl-OC(O)unsubstituted C(₁₋₆)alkyl-NR⁷⁴R⁷⁵, substituted C(₁₋₆)alkyl-C(O)NHS(O)₂R⁷⁶, unsubstituted C(₁₋₆)alkyl-C(O)NHS(O)₂R⁷⁶, substituted C(₆₋₁₁)aryl, substituted C(₃₋₁₁)carbocyclic, substituted C(₄₋₇)heterocarbocycle, substituted C(₄₋₇)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₃₋₁₁)carbocyclic, unsubstituted C(₁₋₁₁)heterocarbocycle, unsubstituted C(₁₋₁₁)heteroaryl, unsubstituted C(₇₋₁₁)aralkyl, and unsubstituted C(₂₋₁₁)heteroaralkyl wherein each of R⁵², R⁵³, R⁷⁴ and R⁷⁵ is selected from the group consisting of: H, unsubstituted C(₁₋₆)alkyl, substituted C(₃₋₇)heterocycloalkyl, unsubstituted C(₃₋₇)heterocycloalkyl, substituted C(₁₋₆)alkyl, substituted C(₃₋₇)cycloalkyl and unsubstituted C(₃₋₇)cycloalkyl, or each pair: a) R⁵² and R⁵³, or (b) R⁷⁴ and R⁷⁵, together form a 3-7 membered substituted heterocarbocyclic ring or a 3-7 membered unsubstituted heterocarbocyclic ring, and wherein each of R⁷¹, R⁷², R⁷³ and R⁷⁶ is independently unsubstituted C(₁₋₁₁)alkyl, or substituted C(₁₋₁₁)alkyl, or
II) together form a 3-7 membered substituted heterocarbocyclic ring or a 3-7 membered unsubstituted heterocarbocyclic ring;
R⁹ is selected from the group consisting of substituted C(₁₋₆)alkyl, substituted C(₁₋₆)alkyl-NR¹⁰R¹¹, unsubstituted C(₁₋₆)alkyl-NR¹⁰R¹¹, substituted C(₁₋₆)alkyl-OR²⁰, unsubstituted C(₁₋₆)alkyl-OR²⁰, and unsubstituted C(₄₋₆)alkyl wherein each of R¹⁰, R¹¹ and R²⁰ is independently selected from the group consisting of: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; R¹⁰ and R¹¹ may alternately as a pair be a 3-7 membered substituted heterocarbocyclic ring or a 3-7 membered unsubstituted heterocarbocyclic ring, or
G⁹ is 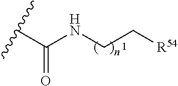
wherein n¹ is 1, 2, 3 or 4 and R⁵⁴ is 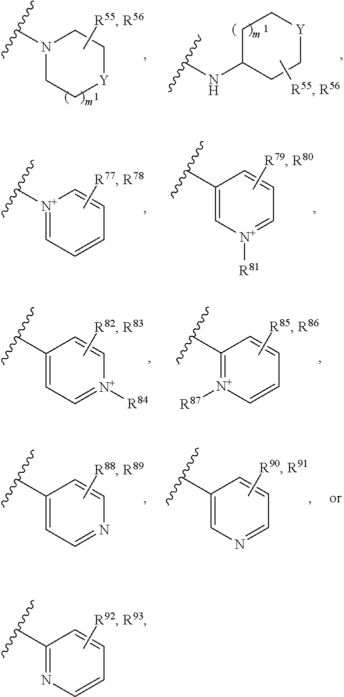
wherein m¹=0, 1 or 2, R⁵⁵ and R⁵⁶ are independently H, carbonyl (═O), Me, Ph, CO₂R⁹⁴, CO₂NH₂, C(₁₋₆)substituted alkyl or C(₁₋₆)unsubstituted alkyl, wherein R⁹⁴ is H, C(₁₋₆)unsubstituted alkyl or C(₁₋₆)substituted alkyl;
R⁷⁷, R⁷⁸, R⁷⁹, R⁸⁰, R⁸², R⁸³, R⁸⁵, R⁸⁶, R⁸⁸, R⁸⁹, R⁹⁰, R⁹¹, R⁹² and R⁹³ are each independently H, C(₁₋₆)substituted alkyl, C(₁₋₆)unsubstituted alkyl, substituted C(₁₋₆)heteroalkyl, unsubstituted C₍₁₋₆₎ heteroalkyl, OR⁹⁵, C(O)R⁹⁶, or NR⁹⁷R⁹⁸, wherein R⁹⁵ is H, C(₁₋₆)substituted alkyl, or C(₁₋₆)unsubstituted alkyl, R⁹⁶ is C(₁₋₆)substituted alkyl, or C(₁₋₆)unsubstituted alkyl, and R⁹⁷ and R⁹⁸ are each independently H, C(₁₋₆)substituted alkyl, or C(₁₋₆)unsubstituted alkyl, or each pair: a) R⁷⁷ and R⁷⁸, b) R⁷⁹ and R⁸⁰, c) R⁸² and R⁸³, d) R⁸⁵ and R⁸⁶, e) R⁸⁸ and R⁸⁹, f) R⁹⁰ and R⁹¹, or g) R⁹² and R⁹³ are attached to adjacent ring-forming C atoms, and together with the ring-forming C atoms, form a substituted C₆ aryl ring or an unsubstituted C₆ aryl ring;
R⁸¹, R⁸⁴ and R⁸⁷ each independently is C(₁₋₆)substituted alkyl, or C(₁₋₆)unsubstituted alkyl; and
Y is CH₂, CHOH, CHO—CO—C(₁₋₆)unsubstituted alkyl, CHO—CO—C(₁₋₆)substituted alkyl, NCONH₂, N—C(₁₋₆)substituted alkyl, N—C(₁₋₆)unsubstituted alkyl, NH or N—C(O)OR⁹⁹ wherein R⁹⁹ is C(₁₋₆)unsubstituted alkyl, C(₁₋₆)substituted alkyl, C(₆₋₁₁)unsubstituted aralkyl or C(₆₋₁₁)substituted aralkyl;
G¹⁰ is selected from the group consisting of: a straight C(₁₋₆)alkyl, a branched C(₃₋₆)alkyl and phenyl;
G¹¹ is NHCH₂, NH, NHCO, SCH₂, O, or S;
G¹² is H, NO₂, or OMe;
G¹³ is H, NO₂, or OMe;
each of G¹⁴, G^{14′} and G¹⁸ is independently NH, S, O, N—CH₃, N—CH₂—OCH₃, N—CH₂—COOH, N—CH₂—CH₂OH, N—CH₂—C(O)NH₂, CH—CH₃, N—R^{14′}, CH—R^{14′} or substituted C(₁₋₆)alkyl-NR⁵²R⁵³, wherein R^{14′} is C₍₁₋₆₎ substituted alkyl, C₍₁₋₆₎ unsubstituted alkyl, 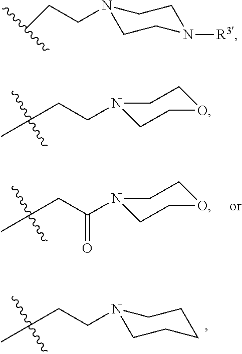
wherein R^{3′} is H, unsubstituted alkyl, or substituted alkyl, wherein the alkyl is 1-6 carbons in length, and the alkyl is optionally substituted with Br, F, Cl, I, OH, OMe, or N₃;
each of G¹⁵, G^{15′} and G¹⁹ is independently N, CH or CG⁹;
G¹⁶ is N or CH;
G¹⁷ is N or CH;
each of n, n², n³ and n⁴ is independently 0, 1, 2, 3 or 4;
each Q¹ and Q¹⁴ is independently selected from the group consisting of: halogen, —OR²⁶, —O—(C₁₋₆)alkyl-NR²⁷R²⁸, —O—(C₁₋₆)alkyl-C(O)OR¹⁰⁰, —O—(C₁₋₆)alkyl-C(O)NHR¹⁰¹, —O—(C₁₋₆)alkyl-OC(O)R¹⁰², —O—(C₁₋₆)alkyl-OS(O)₂R¹⁰³, NO₂, NR¹⁰⁴R¹⁰⁵, —NHC(O)R¹⁰⁶, substituted C(₁₋₆)alkyl, substituted C(₁₋₆)heteroalkyl, unsubstituted C(₁₋₆)alkyl, and unsubstituted C(₁₋₆)heteroalkyl;
each Q² is independently selected from the group consisting of: halogen, —OR²⁹, —O—(C₁₋₆)alkyl-NR³⁰R³¹, —O—(C₁₋₆)alkyl-C(O)OR¹⁰⁷, —O—(CO₁-6)alkyl-C(O)NHR¹⁰⁸, —O—(C₁₋₆)alkyl-OC(O)R¹⁰⁹, —O—(C₁₋₆)alkyl-OS(O)₂R¹¹⁰, NO₂, NR¹¹¹R¹¹², —NHC(O)R¹¹³, substituted C(₁₋₆)alkyl, substituted C(₁₋₆)heteroalkyl, unsubstituted C(₁₋₆)alkyl, and unsubstituted C(₁₋₆)heteroalkyl;
each Q³ is independently selected from the group consisting of: halogen, —OR¹¹⁴, —O—(C₁₋₆)alkyl-NR¹¹⁵R¹¹⁶, —O—(C₁₋₆)alkyl-C(O)OR¹¹⁷, —O—(C₁₋₆)alkyl-C(O)NHR¹¹⁸, —O—(C₁₋₆)alkyl-OC(O)R¹¹⁹, —O—(C₁₋₆)alkyl-OS(O)₂R¹²⁰, NO₂, NR¹²¹R¹²², —NHC(O)R¹²³, substituted C(₁₋₆)alkyl, substituted C(₁₋₆)heteroalkyl, unsubstituted C(₁₋₆)alkyl, and unsubstituted C(₁₋₆)heteroalkyl;
each Q⁴ is independently selected from the group consisting of: halogen, —OR³⁵, —O—(C₁₋₆)alkyl-NR³⁶R³⁷, —O—(C₁₋₆)alkyl-C(O)OR¹²⁴, —O—(C₁₋₆)alkyl-C(O)NHR¹²⁵, —O—(C₁₋₆)alkyl-OC(O)R¹²⁶, —O—(C₁₋₆)alkyl-OS(O)₂R¹²⁷, NO₂, NR¹²⁸R¹²⁹, —NHC(O)R¹³⁰, substituted C(₁₋₆)alkyl, substituted C(₁₋₆)heteroalkyl, unsubstituted C(₁₋₆)alkyl, and unsubstituted C(₁₋₆)heteroalkyl;
each Q⁵ is independently selected from the group consisting of: halogen, —OR³⁸, —O—(C₁₋₆)alkyl-NR³⁹R⁴⁰, —O—(C₁₋₆)alkyl-C(O)OR¹³¹, —O—(C₁₋₆)alkyl-C(O)NHR¹³², —O—(C₁₋₆)alkyl-OC(O)R¹³³, —O—(C₁₋₆)alkyl-OS(O)₂R¹³⁴, NO₂, NR¹³⁵R¹³⁶, —NHC(O)R¹³⁷, substituted C(₁₋₆)alkyl, substituted C(₁₋₆)heteroalkyl, unsubstituted C(₁₋₆)alkyl, and unsubstituted C(₁₋₆)heteroalkyl;
each Q⁶ is independently selected from the group consisting of: halogen, —OR⁴¹, —O—(C₁₋₆)alkyl-NR⁴²R⁴³, —O—(C₁₋₆)alkyl-C(O)OR¹³⁸, —O—(C₁₋₆)alkyl-C(O)NHR¹³⁹, —O—(C₁₋₆)alkyl-OC(O)R¹⁴⁰, —O—(C₁₋₆)alkyl-OS(O)₂R¹⁴¹, NO₂, NR¹⁴²R¹⁴³, —NHC(O)R¹⁴⁴, substituted C(₁₋₆)alkyl, substituted C(₁₋₆)heteroalkyl, unsubstituted C(₁₋₆)alkyl, and unsubstituted C(₁₋₆)heteroalkyl;
each Q⁷ is independently selected from the group consisting of: halogen, —OR⁴⁴, —O—(C₁₋₆)alkyl-NR⁴⁵R⁴⁶, —O—(C₁₋₆)alkyl-C(O)OR¹⁴⁵, —O—(C₁₋₆)alkyl-C(O)NHR¹⁴⁶, —O—(C₁₋₆)alkyl-OC(O)R¹⁴⁷, —O—(C₁₋₆)alkyl-OS(O)₂R¹⁴⁸, NO₂, NR¹⁴⁹R¹⁵⁰, —NHC(O)R¹⁵¹, substituted C(₁₋₆)alkyl, substituted C(₁₋₆)heteroalkyl, unsubstituted C(₁₋₆)alkyl, and unsubstituted C(₁₋₆)heteroalkyl;
each Q⁸ is independently selected from the group consisting of: halogen, —OR⁴⁷, —O—(C₁₋₆)alkyl-NR⁴⁸R⁴⁹, —O—(C₁₋₆)alkyl-C(O)OR¹⁵², —O—(C₁₋₆)alkyl-C(O)NHR¹⁵³, —O—(C₁₋₆)alkyl-OC(O)R¹⁵⁴, —O—(C₁₋₆)alkyl-OS(O)₂R¹⁵⁵, NO₂, NR¹⁵⁶R¹⁵⁷, —NHC(O)R¹⁵⁸, substituted C(₁₋₆)alkyl, substituted C(₁₋₆)heteroalkyl, unsubstituted C(₁₋₆)alkyl, and unsubstituted C(₁₋₆)heteroalkyl;
each Q⁹ is independently selected from the group consisting of: halogen, —OR¹⁵⁹, —O—(C₁₋₆)alkyl-NR¹⁶⁰R¹⁶¹, —O—(C₁₋₆)alkyl-C(O)OR¹⁶², —O—(C₁₋₆)alkyl-C(O)NHR¹⁶³, —O—(C₁₋₆)alkyl-OC(O)R¹⁶⁴, —O—(C₁₋₆)alkyl-OS(O)₂R¹⁶⁵, NO₂, NR¹⁶⁶R¹⁶⁷, —NHC(O)R¹⁶⁸, substituted C(₁₋₆)alkyl, substituted C(₁₋₆)heteroalkyl, unsubstituted C(₁₋₆)alkyl, and unsubstituted C(₁₋₆)heteroalkyl;
each Q¹⁰ is independently selected from the group consisting of:
halogen, —OR¹⁶⁹, —O—(C₁₋₆)alkyl-NR¹⁷⁰R¹⁷¹, —O—(C₁₋₆)alkyl-C(O)OR¹⁷², —O—(C₁₋₆)alkyl-C(O)NHR¹⁷³, —O—(C₁₋₆)alkyl-OC(O)R¹⁷⁴, —O—(C₁₋₆)alkyl-OS(O)₂R¹⁷⁵, NO₂, NR¹⁷⁶R¹⁷⁷, —NHC(O)R¹⁷⁸, substituted C(₁₋₆)alkyl, substituted C(₁₋₆)heteroalkyl, unsubstituted C(₁₋₆)alkyl, and unsubstituted C(₁₋₆)heteroalkyl;
each Q¹¹ is independently selected from the group consisting of:
halogen, —OR¹⁷⁹, —O—(C₁₋₆)alkyl-NR¹⁸⁰R¹⁸¹, —O—(C₁₋₆)alkyl-C(O)OR¹⁸², —O—(C₁₋₆)alkyl-C(O)NHR¹⁸³, —O—(C₁₋₆)alkyl-OC(O)R¹⁸⁴, —O—(C₁₋₆)alkyl-OS(O)₂R¹⁸⁵, NO₂, NR¹⁸⁶R¹⁸⁷, —NHC(O)R¹⁸⁸, substituted C(₁₋₆)alkyl, substituted C(₁₋₆)heteroalkyl, unsubstituted C(₁₋₆)alkyl, and unsubstituted C(₁₋₆)heteroalkyl;
each Q¹² is independently selected from the group consisting of:
halogen, —OR¹⁸⁹, —O—(C₁₋₆)alkyl-NR¹⁹⁰R¹⁹¹, —O—(C₁₋₆)alkyl-C(O)OR¹⁹², —O—(C₁₋₆)alkyl-C(O)NHR¹⁹³, —O—(C₁₋₆)alkyl-OC(O)R¹⁹⁴, —O—(C₁₋₆)alkyl-OS(O)₂R¹⁹⁵, NO₂, NR¹⁹⁶R¹⁹⁷, —NHC(O)R¹⁹⁸, substituted C(₁₋₆)alkyl, substituted C(₁₋₆)heteroalkyl, unsubstituted C(₁₋₆)alkyl, and unsubstituted C(₁₋₆)heteroalkyl;
each Q¹³ is independently selected from the group consisting of:
halogen, —OR¹⁹⁹, —O—(C₁₋₆)alkyl-NR²⁰⁰R²⁰¹, —O—(C₁₋₆)alkyl-C(O)OR²⁰², —O—(C₁₋₆)alkyl-C(O)NHR²⁰³, —O—(C₁₋₆)alkyl-OC(O)R²⁰⁴, —O—(C₁₋₆)alkyl-OS(O)₂R²⁰⁵, NO₂, NR²⁰⁶R²⁰⁷, —NHC(O)R²⁰⁸, substituted C(₁₋₆)alkyl, substituted C(₁₋₆)heteroalkyl, unsubstituted C(₁₋₆)alkyl, and unsubstituted C(₁₋₆)heteroalkyl;
each R²⁶, R²⁷, R²⁸, R²⁹, R³⁰, R³¹, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R¹⁰⁰, R¹⁰⁴, R¹⁰⁵, R¹⁰⁷, R¹¹¹, R¹¹², R¹¹⁴, R¹¹⁵, R¹¹⁶, R¹¹⁷, R¹²¹, R¹²², R¹²⁴, R¹²⁸, R¹²⁹, R¹³¹, R¹³⁵, R¹³⁶, R¹³⁸, R¹⁴², R¹⁴³, R¹⁴⁵, R¹⁴⁹, R¹⁵⁰, R¹⁵², R¹⁵⁶, R¹⁵⁷, R¹⁵⁹, R¹⁶⁰, R¹⁶¹, R¹⁶², R¹⁶⁶, R¹⁶⁷, R¹⁶⁹, R¹⁷⁰, R¹⁷¹, R¹⁷², R¹⁷⁶, R¹⁷⁷, R¹⁷⁹, R¹⁸⁰, R¹⁸¹, R¹⁸², R¹⁸⁶, R¹⁸⁷, R¹⁸⁹, R¹⁹⁰, R^{191′} R¹⁹², R¹⁹⁶, R¹⁹⁷, R¹⁹⁹, R²⁰⁰, R²⁰¹, R²⁰², R²⁰⁶ and R²⁰⁷ are independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and each pair: a) R²⁷ and R²⁸, b) R³⁰ and R³¹, c) R³⁶ and R³⁷, d) R³⁹ and R⁴⁰, e) R⁴² and R⁴³, f) R⁴⁵ and R⁴⁶, g) R⁴⁸ and R⁴⁹, h) R¹⁰⁴ and R¹⁰⁵, i) R¹¹¹ and R¹¹² j) R¹¹⁵ and R¹¹⁶ k) R¹²¹ and R¹²², l) R¹²⁸ and R¹², m) R¹³⁵ and R¹³⁶, n) R¹⁴² and R¹⁴³, o) R¹⁴⁹ and R¹⁵⁰, p) R¹⁵⁶ and R¹⁵⁷ q) R¹⁶⁰ and R¹⁶¹ r) R¹⁶⁶ and R¹⁶⁷ s) R¹⁷⁰ and R¹⁷¹, t) R¹⁷⁶ and R¹⁷⁷, u) R¹⁸⁰ and R¹⁸¹, v) R¹⁸⁶ and R¹⁸⁷, w) R¹⁹⁰ and R¹⁹¹, x) R¹⁹⁶ and R¹⁹⁷, y) R²⁰⁰ and R²⁰¹, and z) R²⁰⁶ and R²⁰⁷ may alternately be and independently as a pair be a 3-7 membered substituted heterocarbocyclic ring or a 3-7 membered unsubstituted heterocarbocyclic ring;
R¹⁰¹, R¹⁰⁸, R¹¹⁸, R¹²⁵, R¹³², R¹³⁹, R¹⁴⁶, R¹⁵³, R¹⁶³, R¹⁷³, R¹⁸³, R¹⁹³ and R²⁰³ are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, unsubstituted C(₂₋₁₁)heteroaralkyl, substituted C(₁₋₆)alkyl-NR²⁰⁹R²¹⁰, unsubstituted C(₁₋₆)alkyl-NR²⁰⁹R²¹⁰, substituted C(₁₋₆)alkyl-N⁺R²¹¹R²¹²R²¹³, unsubstituted C(₁₋₆)alkyl-N⁺R²¹²R²¹³, substituted C(₁₋₆)alkyl-OR²¹⁴, unsubstituted C(₁₋₆)alkyl-OR²¹⁴, 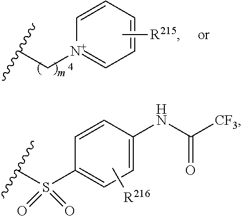
wherein m is 1, 2, 3, 4, or 5, R²⁰⁹, R²¹⁰, R²¹⁴, R²¹⁵ and R²¹⁶ are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and R²⁰⁹ and R²¹⁰, may alternately be and independently as a pair be a 3-7 membered substituted heterocarbocyclic ring or a 3-7 membered unsubstituted heterocarbocyclic ring, and R²¹¹, R²¹² and R²¹³ are each independently unsubstituted C(₁₋₁₁)alkyl, or substituted C(₁₋₁₁)alkyl; and
R¹⁰², R¹⁰³, R¹⁰⁶, R¹⁰⁹, R¹¹⁰, R¹¹³, R¹¹⁹, R¹²⁰, R¹²³, R¹²⁶, R¹²⁷, R¹³⁰, R¹³³, R¹³⁴, R¹³⁷, R¹⁴⁰, R¹⁴¹, R¹⁴⁴, R¹⁴⁷, R¹⁴⁸, R¹⁵¹, R¹⁵⁴, R¹⁵⁵, R¹⁵⁸, R¹⁶⁴, R¹⁶⁵, R¹⁶⁸, R¹⁷⁴, R¹⁷⁵, R¹⁷⁸, R¹⁸⁴, R¹⁸⁵, R¹⁸⁸, R¹⁹⁴, R¹⁹⁵, R¹⁹⁸, R²⁰⁴, R²⁰⁵ and R²⁰⁸ are each independently substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl;
(i) provided that G⁵ is absent only when G², G³ and G⁴ together form the ring moiety 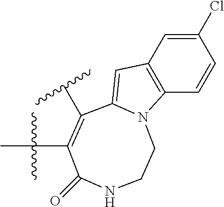 [SMILES: */C1=C(\*)c2cc3cc(Cl)ccc3n2CCNC1=O]
and G⁵ is absent when G², G³ and G⁴ together form the ring moiety 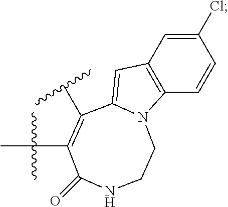 [SMILES: */C1=C(\*)c2cc3cc(Cl)ccc3n2CCNC1=O]
(ii) provided that when G³ is N, CH, or CG⁹ where G⁹ is C(O)OR⁹ and R⁹ is unsubstituted C(₄₋₆) alkyl, G⁴ is other than 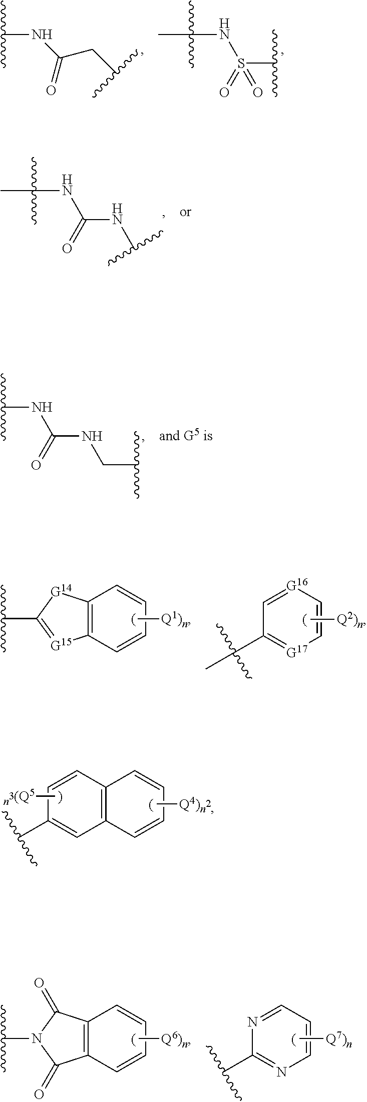
or a 5-membered heteroaryl optionally substituted with (Q⁸)ₙ and containing 1 or 2 heteroatoms each heteroatom independently selected from N, O and S, then n is at least 1 or n²+n³ is at least 1, and
(a) when n is 1 or n²+n³=1, then Q, Q², Q⁴, Q⁵, Q⁶, Q⁷ or Q⁸ is independently selected from the group consisting of —OR^{26′}, —O—(C₁₋₆)alkyl-NR^{27′}R^{28′}, —′O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, and —NHC(O)R^{106′},
wherein R^{26′} is independently selected from the group consisting of substituted C(₁₋₆)alkyl, substituted C(₇₋₁₁)aralkyl, substituted C(₂₋₁₁)heteroaralkyl, unsubstituted C(₅₋₁₁)alkyl, unsubstituted C(₇₋₁₁)aralkyl, and unsubstituted C(₂₋₁₁)heteroaralkyl;
each of R^{27′}, R^{28′}, and R^{100′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; or R^{27′} and R^{28′} may alternately as a pair be a 3-7 membered substituted heterocarbocyclic ring or a 3-7 membered unsubstituted heterocarbocyclic ring;
R^{101′} is H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, unsubstituted C(₂₋₁₁)heteroaralkyl, substituted C(₁₋₆)alkyl-NR^{209′}R^{210′}, unsubstituted C(₁₋₆)alkyl-NR^{209′}R^{210′}, substituted C(₁₋₆)alkyl-N⁺R^{211′}R^{212′}R^{213′}, unsubstituted C(₁₋₆)alkyl-N⁺R^{211′}R^{212′}R^{213′}, substituted C(₁₋₆)alkyl-OR^{214′}, unsubstituted C(₁₋₆)alkyl-OR^{214′}, 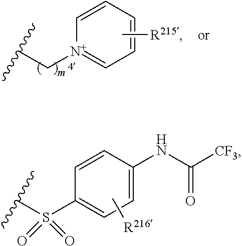
wherein m is 1, 2, 3, 4 or 5, R^{209′}, R^{210′}, R^{214′}, R^{215′} and R^{216′} are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and R^{209′} and R^{210′}, may alternately be and independently as a pair be a 3-7 membered substituted heterocarbocyclic ring or a 3-7 membered unsubstituted heterocarbocyclic ring, and R^{211′}, R^{212′} and R^{213′} are each independently unsubstituted C(₁₋₁₁)alkyl, or substituted C(₁₋₁₁)alkyl; and
R^{102′} and R^{103′} are each independently substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, or unsubstituted C(₂₋₁₁)heteroaralkyl; and
R^{106′} is substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₂₋₁₁)alkyl, unsubstituted C(₆₋₁₁)aryl, unsubstituted C(₁₋₁₁)heteroaryl, unsubstituted C(₇₋₁₁)aralkyl, or unsubstituted C(₂₋₁₁)heteroaralkyl; and
(b) when n is at least 2 or n²+n³ is at least 2, then a first Q¹, Q², Q⁴, Q⁵, Q⁶, Q⁷ or Q⁸ is independently selected from the group consisting of —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, and —NHC(O)R^{106′},
wherein each of R^{26′}, R^{27′}, R^{28′}, R^{100′}, R^{101′}, R^{102′}, R^{103′}, and R^{106′} is as defined above; and
the remaining Q¹, Q², Q⁴, Q⁵, Q⁶, Q⁷ or Q⁸ are each independently selected from the group consisting of halogen, —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, NR^{104′}R^{105′}, —NHC(O)R^{106′}, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
wherein each R^{26′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl;
each of R^{104′} and R^{105′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; or R^{104′} and R^{105′} may alternately as a pair be a 3-7 membered substituted heterocarbocyclic ring or a 3-7 membered unsubstituted heterocarbocyclic ring;
each R^{106′} is substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, or unsubstituted C(₂₋₁₁)heteroaralkyl; and
each of R^{27′}, R^{28′}, R^{100′} R^{101′}, R^{102′}, and R^{103′} is as defined above;
(iii) provided that when G³ is N, CH, or CG⁹ where G⁹ is C(O)OR⁹ and R⁹ is unsubstituted C(₄₋₆) alkyl, G⁴ is other than 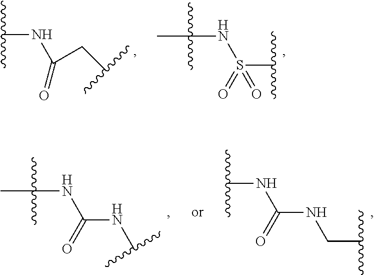 [SMILES: *CC(=O)N* ; *NS(*)(=O)=O ; *NC(=O)N* ; *CNC(=O)N*]
and G⁵ is 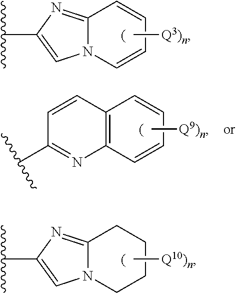
then at least one of G⁶, G⁷, and G⁸ is not H; n is at least 1; and each of Q³, Q⁹ or Q¹⁰ is independently selected from the group consisting of halogen, —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, —NHC(O)R^{106′}, substituted C₍₁₋₆₎alkyl, substituted C₍₁₋₆₎heteroalkyl, unsubstituted C₍₂₋₆₎alkyl, and unsubstituted C₍₁₋₆₎heteroalkyl;
(iv) provided that when G³ is N or CH, and G⁵ is, 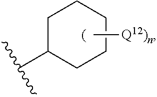
then at least one of G⁶, G⁷, and G⁸ is not H; n is at least 1; and each Q is independently selected from the group consisting of halogen, —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, —NHC(O)R^{106′}, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
(v) provided that when G³ is N or CH, and G⁴ is 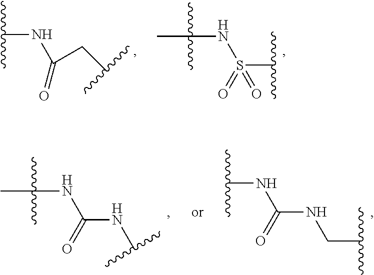 [SMILES: *CC(=O)N* ; *NS(*)(=O)=O ; *NC(=O)N* ; *CNC(=O)N*]
and G⁵ is: (a) 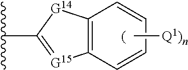
where G¹⁴ is CH₂ and G¹⁵ is N, or G¹⁴ is NH and G¹⁵ is CH, or G¹⁴ is S and G¹⁵ is CH; (b) 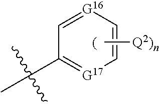
where G¹⁶ is N and G¹⁷ is N; or (c) 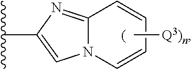
then at least one of G⁶, G⁷, and G⁸ is not H, and n is at least 1;
(vi) provided that when G³ is N or CH, and G⁴ is 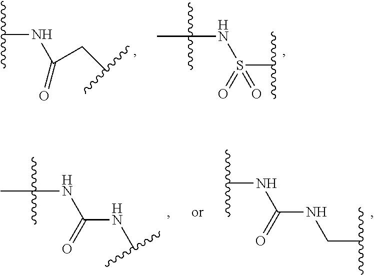 [SMILES: *CC(=O)N* ; *NS(*)(=O)=O ; *NC(=O)N* ; *CNC(=O)N*]
and G⁵ is: (a) 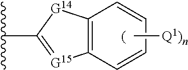
where G¹⁴ is NH and G¹⁵ is N; (b) 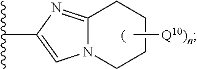
(c) 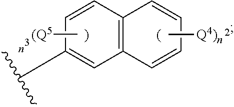
or (d) 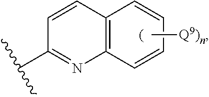
then at least one of G⁶, G⁷ and G⁸ is not H, and each of G⁶ and G⁷ is independently H, halogen, CF₃, NO₂, substituted (C₁₋₁₁)alkyl, unsubstituted (C₃₋₁₁)alkyl, substituted (C₁₋₁₁)alkoxyl, unsubstituted (C₁₋₁₁) alkoxyl, substituted (C₆₋₁₁)aryloxy, unsubstituted (C₆₋₁₁)aryloxy, C(O)OR⁵⁰, or 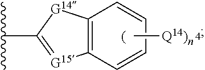
n is at least 1 or n²+n³ is at least 1; and each of Q¹, Q⁴, Q⁵, Q⁹, Q¹⁰ and Q¹² is independently selected from the group consisting of halogen, —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, —NHC(O)R^{106′}, substituted C₍₁₋₆₎alkyl, and unsubstituted C₍₂₋₆₎alkyl;
R^{106′} is substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₂₋₁₁)alkyl, unsubstituted C(₆₋₁₁)aryl, unsubstituted C(₁₋₁₁)heteroaryl, unsubstituted C(₇₋₁₁)aralkyl, or unsubstituted C(₂₋₁₁)heteroaralkyl; and
each of R^{26′}, R^{27′}, R^{28′}, R^{100′}, R^{101′}, R^{102′}, and R^{103′} is as defined above;
(vii) provided that when G³ is N or CH, and G⁴ is 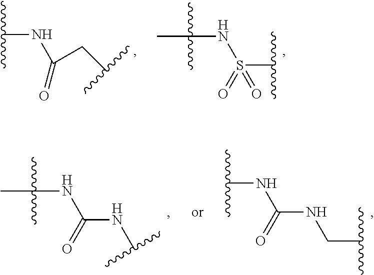 [SMILES: *CC(=O)N* ; *NS(*)(=O)=O ; *NC(=O)N* ; *CNC(=O)N*]
and G⁵ is: (a) 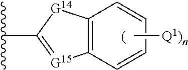
where G¹⁴ is S and G¹⁵ is N; (b) 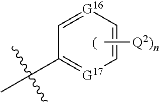
where G¹⁶ is CH and G¹⁷ is N, or G¹⁶ is N and G¹⁷ is CH, or G¹⁶ is CH and G¹⁷ is CH; (c) 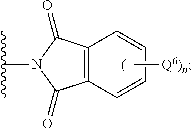
or (d) a 5-membered heteroaryl optionally substituted with (Q⁸)ₙ and containing 1 or 2 heteroatoms each heteroatom independently selected from N, O and S, then at least one of G⁶, G⁷ and G⁸ is not H, and each of G⁶ and G⁷ is independently H, halogen, CF₃, NO₂, substituted (C₁₋₁₁)alkyl, unsubstituted (C₃₋₁₁)alkyl, substituted (C₁₋₁₁)alkoxyl, unsubstituted (C₁₋₁₁) alkoxyl, substituted (C₆₋₁₁)aryloxy, unsubstituted (C₆₋₁₁)aryloxy, C(O)OR⁵⁰, or 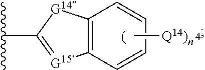
and n is at least 1; and
(a) when n is 1, then each of Q¹, Q², Q⁶, or Q⁸ is independently selected from the group consisting of —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, and —NHC(O)R^{106′},
wherein each of R^{26′}, R^{27′}, R^{28′}, R^{100′}, R^{101′}, R^{102′}, R^{103′} and R^{106′} is as defined above; and
(b) when n is at least 2, then a first Q¹, Q², Q⁶, or Q⁸ is independently selected from the group consisting of —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, and —NHC(O)R^{106′},
wherein each of R²⁶, R²⁷, R²⁸, R^{100′}, R^{101′}, R^{102′}, R^{103′}, and R^{106′} is as defined above; and
the remaining Q¹, Q², Q⁶, or Q⁸ are each independently selected from the group consisting of halogen, —OR^{26′}, —O—(C₁₋₆)alkyl-NR^{27′}R^{28′}, —O—(C₁₋₆)alkyl-C(O)OR¹⁰⁰, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, NR^{104′}R^{105′}, —NHC(O)R^{106′}, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
wherein each R^{26′}, R^{27′}, R^{28′}, R^{100′}, R^{101′}, R^{102′}, R^{103′}, R^{104′}, R^{105′}, and R^{106′} is as defined above; and
(viii) provided that when G³ is CG⁹ and G⁹ is: (a) substituted (C₁₋₆) alkyl-NH₂; (b) unsubstituted (C₁₋₆) alkyl-NH₂; (c) substituted (C₁₋₆) alkyl-NR⁶⁴R⁶⁵ or unsubstituted (C₁₋₆) alkyl-NR⁶⁴R⁶⁵ where R⁶⁴ and R⁶⁵ as a pair are a 3-7 membered substituted heterocarbocyclic ring or a 3-7 membered unsubstituted heterocarbocyclic ring; (d) substituted (C₆₋₁₁) aryl; (e) substituted (C₁₋₁₁) heteroaryl or unsubstituted (C₁₋₁₁) heteroaryl; (f) substituted (C₆₋₁₁) arylcarbonyl or unsubstituted (C₆₋₁₁) arylcarbonyl; (g) substituted (C₁₋₁₁) heteroarylcarbonyl or unsubstituted (C₁₋₁₁) heteroarylcarbonyl;
(h) —CO-substituted-carbocycle or —CO-unsubstituted-carbocycle;
(i) —CO-substituted-heterocarbocycle or —CO-unsubstituted-heterocarbocycle;
(j) —C(O)NR⁷R⁸ where each of R⁷ and R⁸ is CH₃; (k) —C(O)NR⁷R⁸ where R⁷ is H and R⁸ is unsubstituted C₆ aryl or unsubstituted C₄ cycloalkyl; (l) —C(O)C(O)NR¹³R¹⁴ where each of R¹³ and R¹⁴ is CH₃; (m) —C(O)C(O)NR¹³R¹⁴ where each of R¹³ and R¹⁴ is 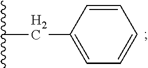 [SMILES: *Cc1ccccc1]
(n) —NR¹⁵R¹⁶ where only one of R¹⁵ and R¹⁶ is unsubstituted C₆ aryl; or (o) —NR¹⁵R¹⁶ where R¹⁵ and R¹⁶ as a pair are a 3-7 membered unsubstituted heterocarbocyclic ring, then at least one of G⁶, G⁷ and G⁸ is not H.

2. The compound of claim 1 wherein G¹ is NH or S.

3. The compound of claim 1 wherein G⁴ is selected from the group consisting of: a bond,

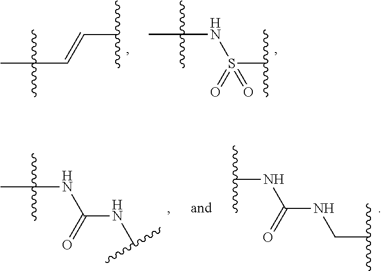

4. The compound of claim 1 wherein G⁴ is selected from the group consisting of

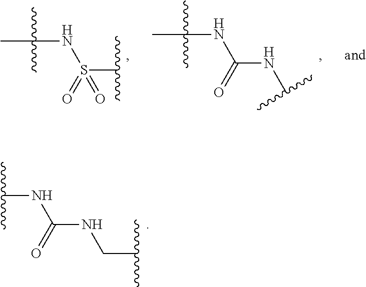 [SMILES: *NS(*)(=O)=O ; *NC(=O)N* ; *CNC(=O)N*]

5. The compound of claim 1 wherein G⁴ is selected from the group consisting of: a bond, and

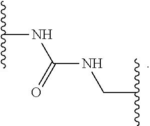 [SMILES: *CNC(=O)N*]

6. The compound of claim 1 wherein G¹ is S and G⁴ is

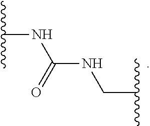 [SMILES: *CNC(=O)N*]

7. The compound of claim 1 wherein G¹ is NH, and G⁴ is a bond.

8. The compound of claim 7 wherein G³ is CG⁹ or CH.

9. The compound of claim 8 wherein G⁵ is

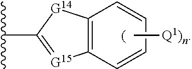

10. The compound of claim 9 wherein G¹⁴ is NH and G¹⁵ is CH.

11. The compound of claim 10 wherein n is at least 2.

12. The compound of claim 11 wherein at least one Q¹ is selected from the group consisting of: —OR²⁶, —O—(C₁₋₆)alkyl-NR²⁷R²⁸, —O—(C₁₋₆)alkyl-C(O)OR¹⁰⁰, —O—(C₁₋₆)alkyl-C(O)NHR¹⁰¹, —O—(C₁₋₆)alkyl-OC(O)R¹⁰², and —O—(C₁₋₆)alkyl-OS(O)₂R¹⁰³.

13. The compound of claim 12 wherein at least one Q¹ is halogen.

14. The compound of claim 13 wherein at least one Q¹ is —O—(C₁₋₆)alkyl-C(O)NHR¹⁰¹.

15. The compound of claim 14 wherein R¹⁰¹ is selected from the group consisting of: unsubstituted C(₁₋₆)alkyl-NR²⁰⁹R²¹⁰, unsubstituted C(₁₋₆)alkyl-N⁺R²¹¹R²¹²R²¹³, unsubstituted C(₁₋₆)alkyl-OR²¹⁴, and

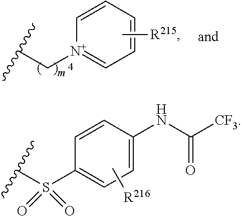

16. The compound of claim 15 wherein at least one Q¹ is Cl.

17. The compound of any one of claims 11 to 16 wherein n is 2.

18. The compound of claim 10 wherein n is at least 1.

19. The compound of claim 18 wherein at least 1 Q¹ is a halogen.

20. The compound of claim 19 wherein G⁹ is —C(NOH)C(R²¹)(R²²)(R²³) or C(NOH)N(R²⁴)(R²⁵).

21. The compound of claim 20 wherein R²¹, R²² and R²³ are each F.

22. The compound of claim 20 wherein R²⁴ and R²⁵ are H.

23. The compound of claim 6 wherein G⁵ is selected from the group consisting of:

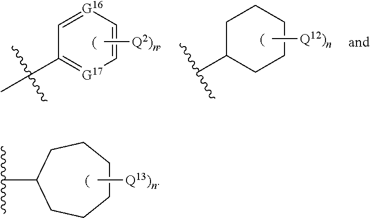

24. The compound of claim 23 wherein G⁵ is

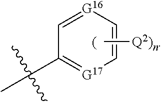

25. The compound of claim 24 wherein G¹⁶ is CH and G¹⁷ is CH.

26. The compound of claim 25 wherein n is 0, 1 or 2.

27. The compound of claim 26 wherein n is at least one 1 and Q² is is selected from the group consisting of: halogen, NR¹¹¹R¹¹², NHC(O)R¹¹³, and substituted C₍₁₋₆₎ alkyl.

28. The compound of claim 27 wherein the substituted C₍₁₋₆₎ alkyl is a halogen substituted methyl group.

29. The compound of claim 28 wherein the halogen substituted methyl group is CF₃.

30. The compound of any one of claims 26 to 29 wherein n is 1.

31. The compound of any one of claims 26 to 29 wherein n is 2.

32. The compound of any one of claims 27 to 31 wherein at least one Q² is halogen.

33. A compound selected from the group consisting of:
TABLE 1

| Compound | |
|---|---|
| # | Chemical Structure |
| 1 | 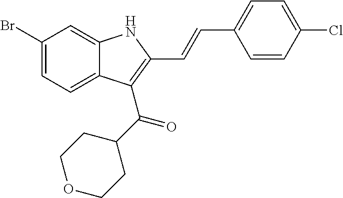 [SMILES: O=C(c1c(/C=C/c2ccc(Cl)cc2)[nH]c2cc(Br)ccc12)C1CCOCC1] |
| 2 | 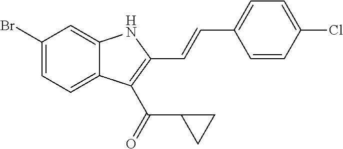 [SMILES: O=C(c1c(/C=C/c2ccc(Cl)cc2)[nH]c2cc(Br)ccc12)C1CC1] |
| 3 | 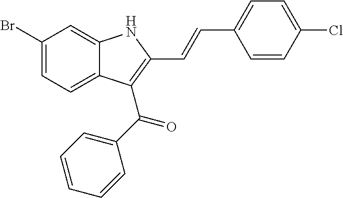 [SMILES: O=C(c1ccccc1)c1c(/C=C/c2ccc(Cl)cc2)[nH]c2cc(Br)ccc12] |
| 4 | 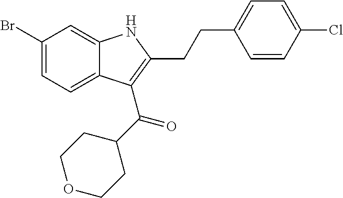 [SMILES: O=C(c1c(CCc2ccc(Cl)cc2)[nH]c2cc(Br)ccc12)C1CCOCC1] |
| 5 | 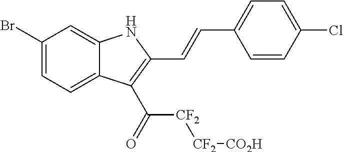 [SMILES: O=C(O)C(F)(F)C(F)(F)C(=O)c1c(/C=C/c2ccc(Cl)cc2)[nH]c2cc(Br)ccc12] |
| 6 | 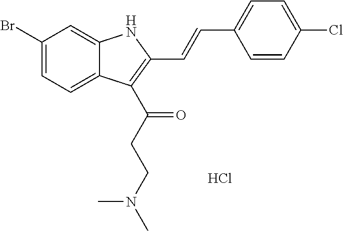 [SMILES: CN(C)CCC(=O)c1c(/C=C/c2ccc(Cl)cc2)[nH]c2cc(Br)ccc12 ; Cl] |
| 7 | 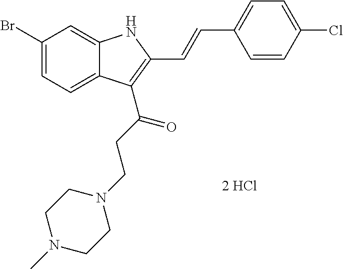 [SMILES: CN1CCN(CCC(=O)c2c(/C=C/c3ccc(Cl)cc3)[nH]c3cc(Br)ccc23)CC1] |
| 8 | 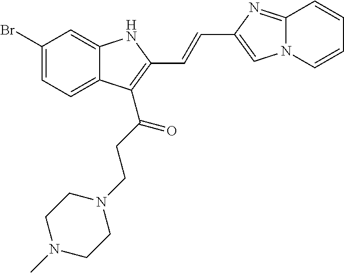 [SMILES: CN1CCN(CCC(=O)c2c(/C=C/c3cn4ccccc4n3)[nH]c3cc(Br)ccc23)CC1] |
| 9 | 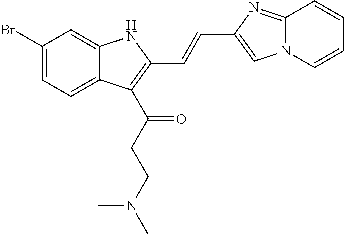 [SMILES: CN(C)CCC(=O)c1c(/C=C/c2cn3ccccc3n2)[nH]c2cc(Br)ccc12] |
| 10 | 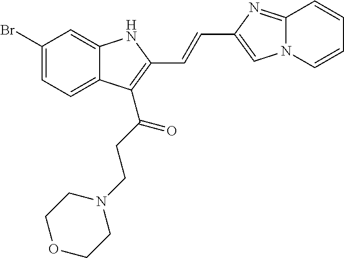 [SMILES: O=C(CCN1CCOCC1)c1c(/C=C/c2cn3ccccc3n2)[nH]c2cc(Br)ccc12] |
| 11 | 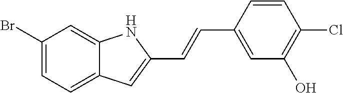 [SMILES: Oc1cc(/C=C/c2cc3ccc(Br)cc3[nH]2)ccc1Cl] |
| 12 | 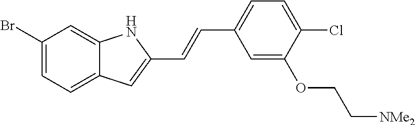 [SMILES: CN(C)CCOc1cc(/C=C/c2cc3ccc(Br)cc3[nH]2)ccc1Cl] |
| 13 | 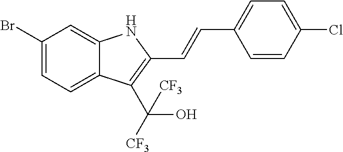 [SMILES: OC(c1c(/C=C/c2ccc(Cl)cc2)[nH]c2cc(Br)ccc12)(C(F)(F)F)C(F)(F)F] |
| 14 | 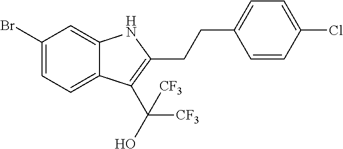 [SMILES: OC(c1c(CCc2ccc(Cl)cc2)[nH]c2cc(Br)ccc12)(C(F)(F)F)C(F)(F)F] |
| 15 | 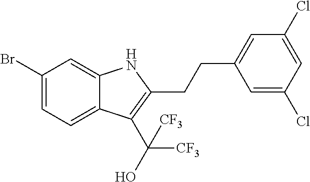 [SMILES: OC(c1c(CCc2cc(Cl)cc(Cl)c2)[nH]c2cc(Br)ccc12)(C(F)(F)F)C(F)(F)F] |
| 16 | 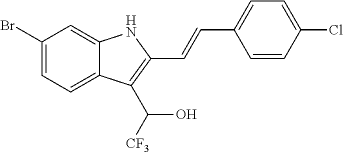 [SMILES: OC(c1c(/C=C/c2ccc(Cl)cc2)[nH]c2cc(Br)ccc12)C(F)(F)F] |
| 17 | 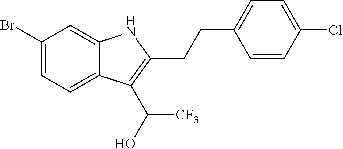 [SMILES: OC(c1c(CCc2ccc(Cl)cc2)[nH]c2cc(Br)ccc12)C(F)(F)F] |
| 18 | 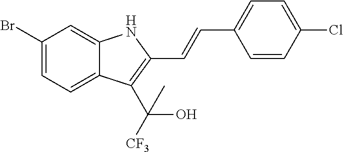 [SMILES: CC(O)(c1c(/C=C/c2ccc(Cl)cc2)[nH]c2cc(Br)ccc12)C(F)(F)F] |
| 19 | 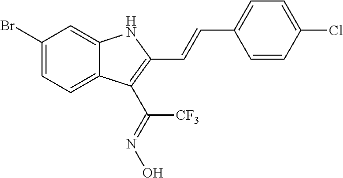 [SMILES: O/N=C(/c1c(/C=C/c2ccc(Cl)cc2)[nH]c2cc(Br)ccc12)C(F)(F)F] |
| 20 | 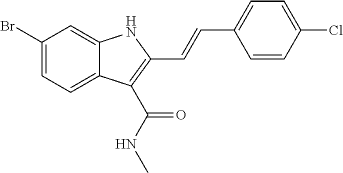 [SMILES: CNC(=O)c1c(/C=C/c2ccc(Cl)cc2)[nH]c2cc(Br)ccc12] |
| 21 | 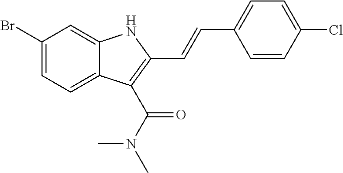 [SMILES: CN(C)C(=O)c1c(/C=C/c2ccc(Cl)cc2)[nH]c2cc(Br)ccc12] |
| 22 | 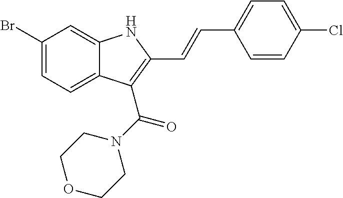 [SMILES: O=C(c1c(/C=C/c2ccc(Cl)cc2)[nH]c2cc(Br)ccc12)N1CCOCC1] |
| 23 | 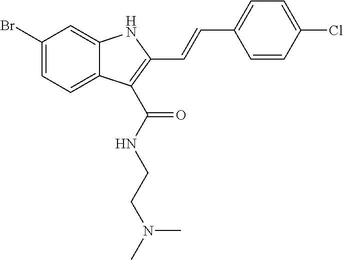 [SMILES: CN(C)CCNC(=O)c1c(/C=C/c2ccc(Cl)cc2)[nH]c2cc(Br)ccc12] |
| 24 | 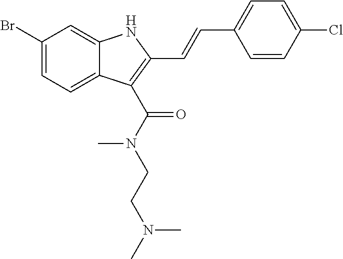 [SMILES: CN(C)CCN(C)C(=O)c1c(/C=C/c2ccc(Cl)cc2)[nH]c2cc(Br)ccc12] |
| 25 | 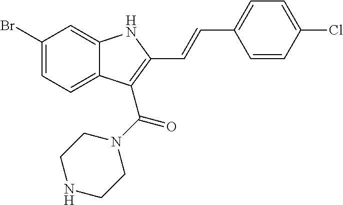 [SMILES: O=C(c1c(/C=C/c2ccc(Cl)cc2)[nH]c2cc(Br)ccc12)N1CCNCC1] |
| 26 | 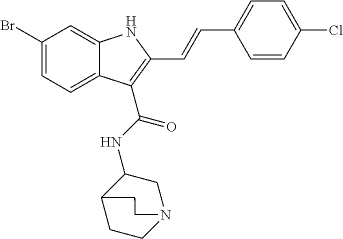 [SMILES: O=C(NC1CN2CCC1CC2)c1c(/C=C/c2ccc(Cl)cc2)[nH]c2cc(Br)ccc12] |
| 27 | 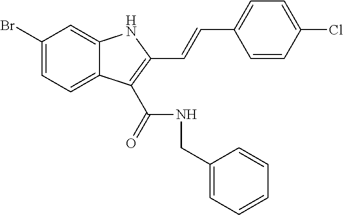 [SMILES: O=C(NCc1ccccc1)c1c(/C=C/c2ccc(Cl)cc2)[nH]c2cc(Br)ccc12] |
| 28 | 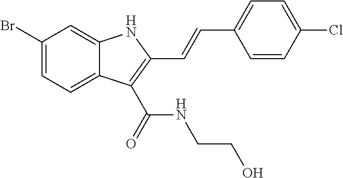 [SMILES: O=C(NCCO)c1c(/C=C/c2ccc(Cl)cc2)[nH]c2cc(Br)ccc12] |
| 29 | 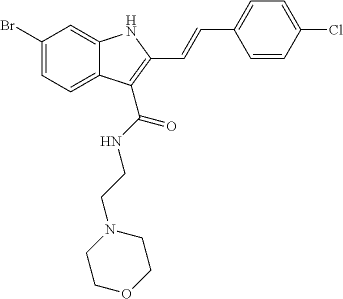 [SMILES: O=C(NCCN1CCOCC1)c1c(/C=C/c2ccc(Cl)cc2)[nH]c2cc(Br)ccc12] |
| 30 | 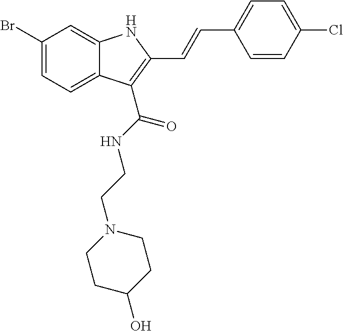 [SMILES: O=C(NCCN1CCC(O)CC1)c1c(/C=C/c2ccc(Cl)cc2)[nH]c2cc(Br)ccc12] |
| 31 | 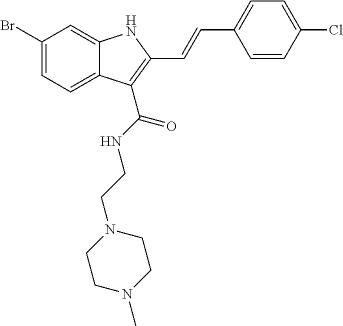 [SMILES: CN1CCN(CCNC(=O)c2c(/C=C/c3ccc(Cl)cc3)[nH]c3cc(Br)ccc23)CC1] |
| 32 | 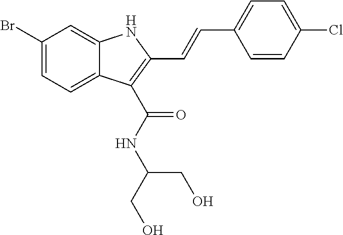 [SMILES: O=C(NC(CO)CO)c1c(/C=C/c2ccc(Cl)cc2)[nH]c2cc(Br)ccc12] |
| 33 | 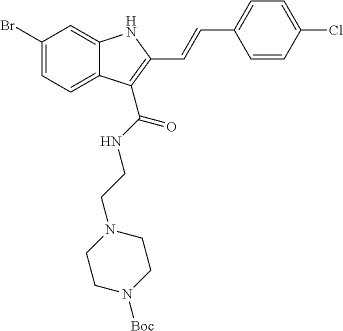 [SMILES: CC(C)(C)OC(=O)N1CCN(CCNC(=O)c2c(/C=C/c3ccc(Cl)cc3)[nH]c3cc(Br)ccc23)CC1] |
| 34 | 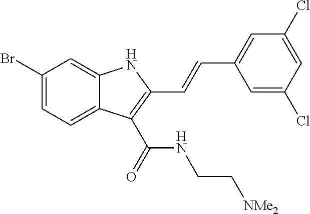 [SMILES: CN(C)CCNC(=O)c1c(/C=C/c2cc(Cl)cc(Cl)c2)[nH]c2cc(Br)ccc12] |
| 35 | 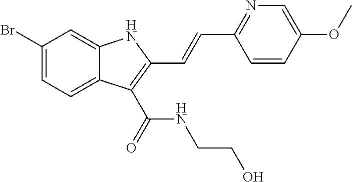 [SMILES: COc1ccc(/C=C/c2[nH]c3cc(Br)ccc3c2C(=O)NCCO)nc1] |
| 36 | 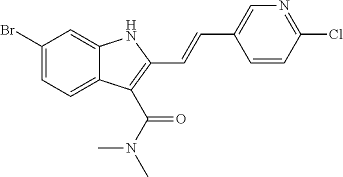 [SMILES: CN(C)C(=O)c1c(/C=C/c2ccc(Cl)nc2)[nH]c2cc(Br)ccc12] |
| 37 | 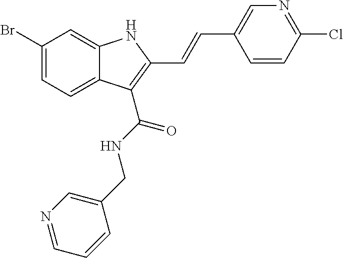 [SMILES: O=C(NCc1cccnc1)c1c(/C=C/c2ccc(Cl)nc2)[nH]c2cc(Br)ccc12] |
| 38 | 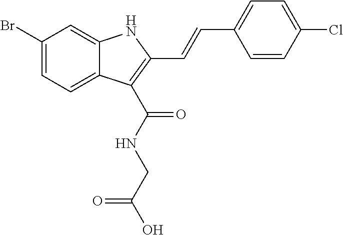 [SMILES: O=C(O)CNC(=O)c1c(/C=C/c2ccc(Cl)cc2)[nH]c2cc(Br)ccc12] |
| 39 | 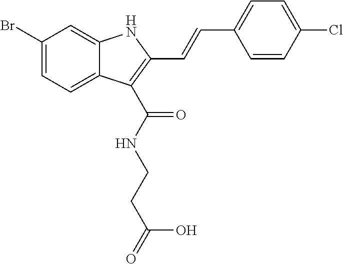 [SMILES: O=C(O)CCNC(=O)c1c(/C=C/c2ccc(Cl)cc2)[nH]c2cc(Br)ccc12] |
| 40 | 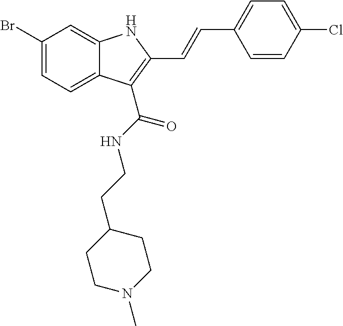 [SMILES: CN1CCC(CCNC(=O)c2c(/C=C/c3ccc(Cl)cc3)[nH]c3cc(Br)ccc23)CC1] |
| 41 | 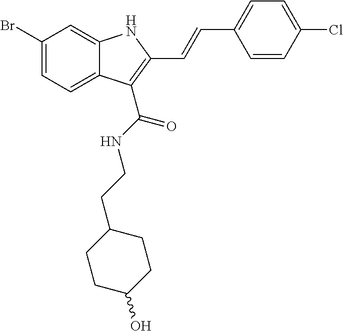 [SMILES: O=C(NCCC1CCC(O)CC1)c1c(/C=C/c2ccc(Cl)cc2)[nH]c2cc(Br)ccc12] |
| 42 | 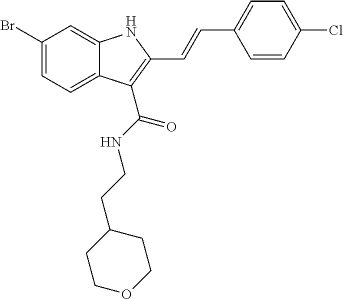 [SMILES: O=C(NCCC1CCOCC1)c1c(/C=C/c2ccc(Cl)cc2)[nH]c2cc(Br)ccc12] |
| 43 | 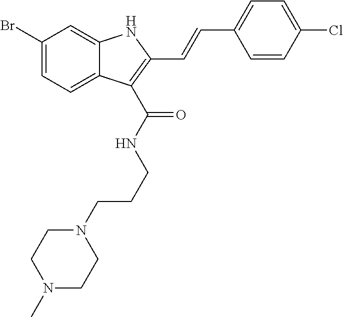 [SMILES: CN1CCN(CCCNC(=O)c2c(/C=C/c3ccc(Cl)cc3)[nH]c3cc(Br)ccc23)CC1] |
| 44 | 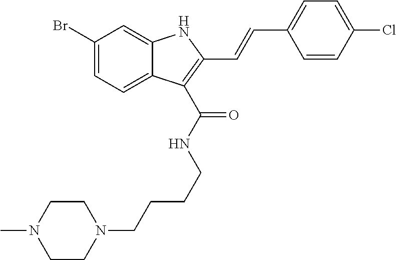 [SMILES: CN1CCN(CCCCNC(=O)c2c(/C=C/c3ccc(Cl)cc3)[nH]c3cc(Br)ccc23)CC1] |
| 45 | 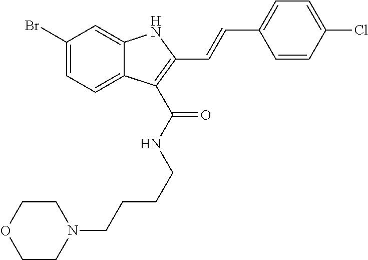 [SMILES: O=C(NCCCCN1CCOCC1)c1c(/C=C/c2ccc(Cl)cc2)[nH]c2cc(Br)ccc12] |
| 46 | 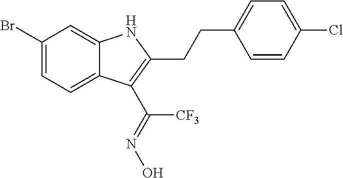 [SMILES: O/N=C(/c1c(CCc2ccc(Cl)cc2)[nH]c2cc(Br)ccc12)C(F)(F)F] |
| 47 | 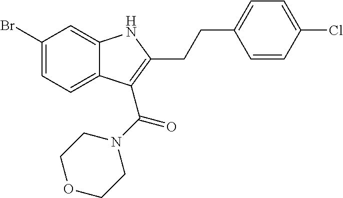 [SMILES: O=C(c1c(CCc2ccc(Cl)cc2)[nH]c2cc(Br)ccc12)N1CCOCC1] |
| 48 | 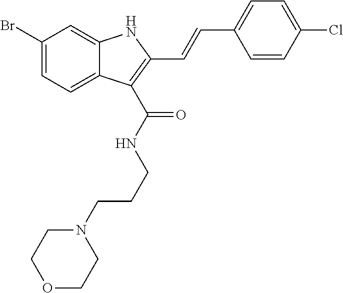 [SMILES: O=C(NCCCN1CCOCC1)c1c(/C=C/c2ccc(Cl)cc2)[nH]c2cc(Br)ccc12] |
| 49 | 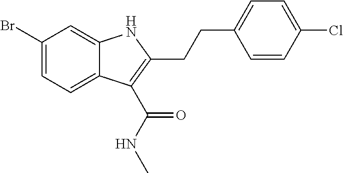 [SMILES: CNC(=O)c1c(CCc2ccc(Cl)cc2)[nH]c2cc(Br)ccc12] |
| 50 | 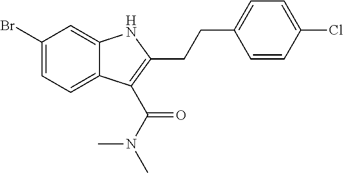 [SMILES: CN(C)C(=O)c1c(CCc2ccc(Cl)cc2)[nH]c2cc(Br)ccc12] |
| 51 | 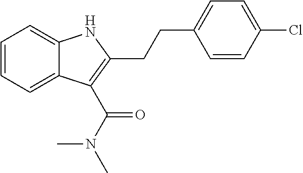 [SMILES: CN(C)C(=O)c1c(CCc2ccc(Cl)cc2)[nH]c2ccccc12] |
| 52 | 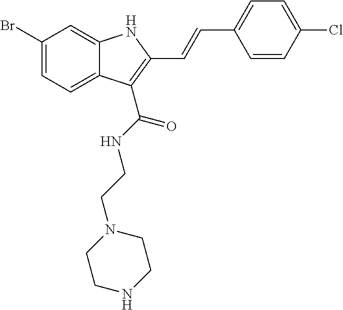 [SMILES: O=C(NCCN1CCNCC1)c1c(/C=C/c2ccc(Cl)cc2)[nH]c2cc(Br)ccc12] |
| 53 | 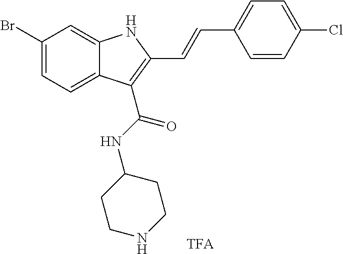 [SMILES: O=C(NC1CCNCC1)c1c(/C=C/c2ccc(Cl)cc2)[nH]c2cc(Br)ccc12] |
| 54 | 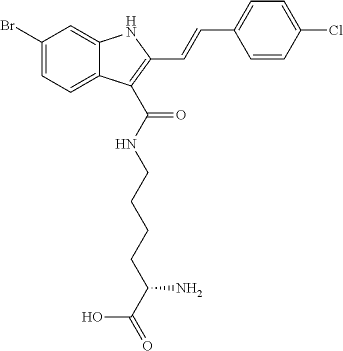 [SMILES: N[C@@H](CCCCNC(=O)c1c(/C=C/c2ccc(Cl)cc2)[nH]c2cc(Br)ccc12)C(=O)O] |
| 55 | 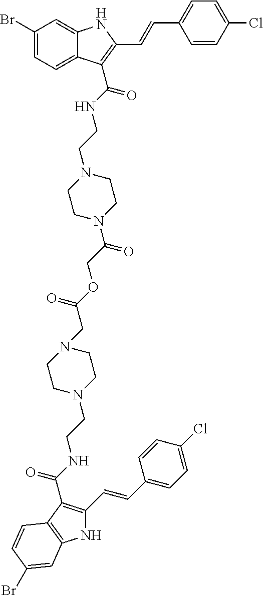 [SMILES: O=C(CN1CCN(CCNC(=O)c2c(/C=C/c3ccc(Cl)cc3)[nH]c3cc(Br)ccc23)CC1)OCC(=O)N1CCN(CCNC(=O)c2c(/C=C/c3ccc(Cl)cc3)[nH]c3cc(Br)ccc23)CC1] |
| 56 | 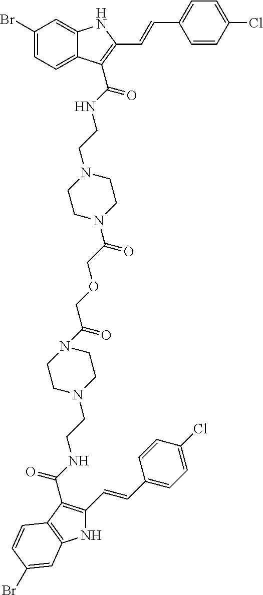 [SMILES: O=C(NCCN1CCN(C(=O)COCC(=O)N2CCN(CCNC(=O)c3c(/C=C/c4ccc(Cl)cc4)[nH]c4cc(Br)ccc34)CC2)CC1)c1c(/C=C/c2ccc(Cl)cc2)[nH]c2cc(Br)ccc12] |
| 57 | 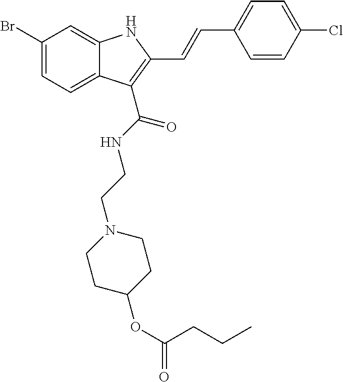 [SMILES: CCCC(=O)OC1CCN(CCNC(=O)c2c(/C=C/c3ccc(Cl)cc3)[nH]c3cc(Br)ccc23)CC1] |
| 58 | 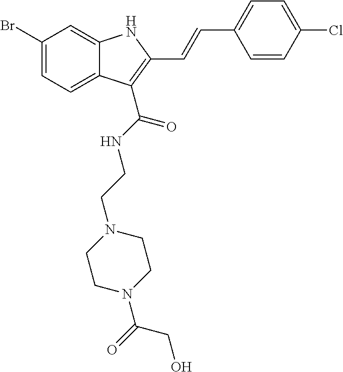 [SMILES: O=C(NCCN1CCN(C(=O)CO)CC1)c1c(/C=C/c2ccc(Cl)cc2)[nH]c2cc(Br)ccc12] |
| 59 | 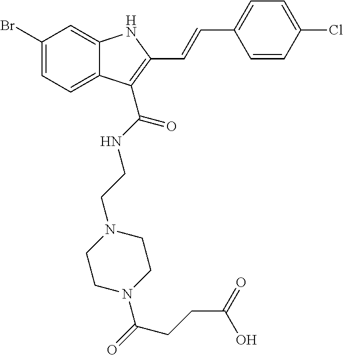 [SMILES: O=C(O)CCC(=O)N1CCN(CCNC(=O)c2c(/C=C/c3ccc(Cl)cc3)[nH]c3cc(Br)ccc23)CC1] |
| 60 | 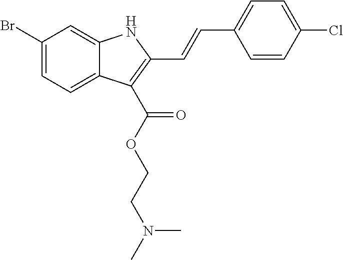 [SMILES: CN(C)CCOC(=O)c1c(/C=C/c2ccc(Cl)cc2)[nH]c2cc(Br)ccc12] |
| 61 | 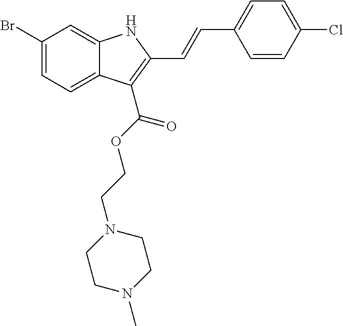 [SMILES: CN1CCN(CCOC(=O)c2c(/C=C/c3ccc(Cl)cc3)[nH]c3cc(Br)ccc23)CC1] |
| 62 | 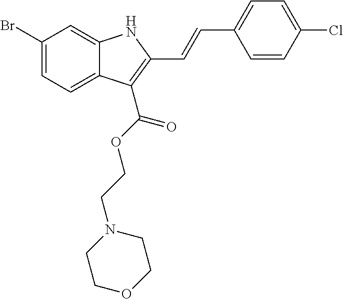 [SMILES: O=C(OCCN1CCOCC1)c1c(/C=C/c2ccc(Cl)cc2)[nH]c2cc(Br)ccc12] |
| 63 | 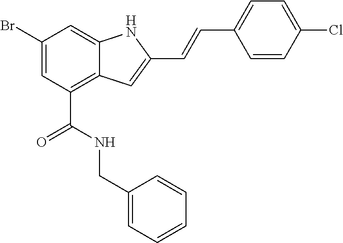 [SMILES: O=C(NCc1ccccc1)c1cc(Br)cc2[nH]c(/C=C/c3ccc(Cl)cc3)cc12] |
| 64 | 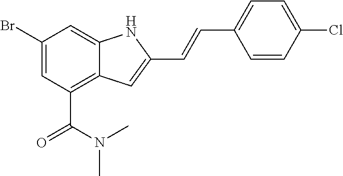 [SMILES: CN(C)C(=O)c1cc(Br)cc2[nH]c(/C=C/c3ccc(Cl)cc3)cc12] |
| 65 | 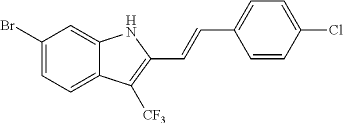 [SMILES: FC(F)(F)c1c(/C=C/c2ccc(Cl)cc2)[nH]c2cc(Br)ccc12] |
| 66 | 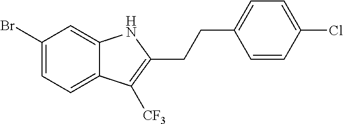 [SMILES: FC(F)(F)c1c(CCc2ccc(Cl)cc2)[nH]c2cc(Br)ccc12] |
| 67 | 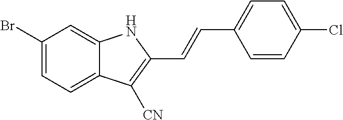 [SMILES: N#Cc1c(/C=C/c2ccc(Cl)cc2)[nH]c2cc(Br)ccc12] |
| 68 | 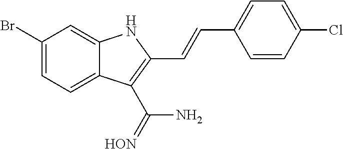 |
| 69 | 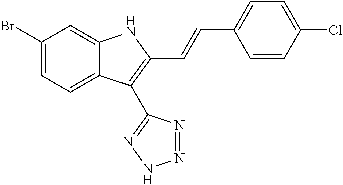 [SMILES: Clc1ccc(/C=C/c2[nH]c3cc(Br)ccc3c2-c2nn[nH]n2)cc1] |
| 70 | 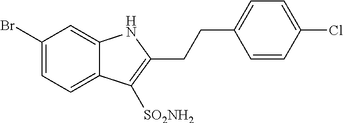 [SMILES: NS(=O)(=O)c1c(CCc2ccc(Cl)cc2)[nH]c2cc(Br)ccc12] |
| 71 | 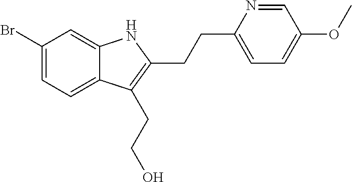 [SMILES: COc1ccc(CCc2[nH]c3cc(Br)ccc3c2CCO)nc1] |
| 72 | 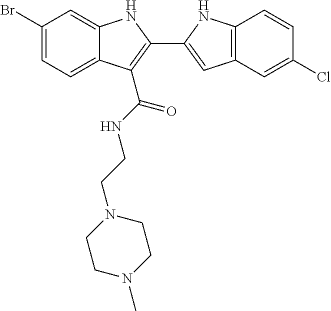 [SMILES: CN1CCN(CCNC(=O)c2c(-c3cc4cc(Cl)ccc4[nH]3)[nH]c3cc(Br)ccc23)CC1] |
| 73 | 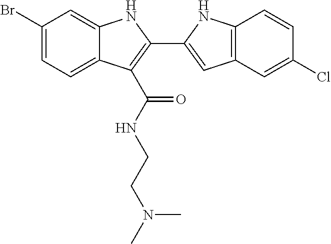 [SMILES: CN(C)CCNC(=O)c1c(-c2cc3cc(Cl)ccc3[nH]2)[nH]c2cc(Br)ccc12] |
| 74 | 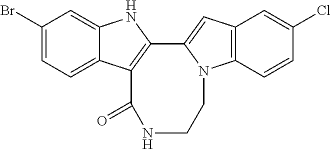 [SMILES: O=C1NCCn2c(cc3cc(Cl)ccc32)-c2[nH]c3cc(Br)ccc3c21] |
| 75 | 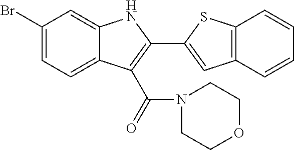 [SMILES: O=C(c1c(-c2cc3ccccc3s2)[nH]c2cc(Br)ccc12)N1CCOCC1] |
| 76 | 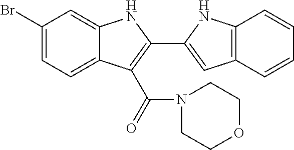 [SMILES: O=C(c1c(-c2cc3ccccc3[nH]2)[nH]c2cc(Br)ccc12)N1CCOCC1] |
| 77 | 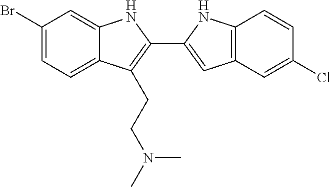 [SMILES: CN(C)CCc1c(-c2cc3cc(Cl)ccc3[nH]2)[nH]c2cc(Br)ccc12] |
| 78 | 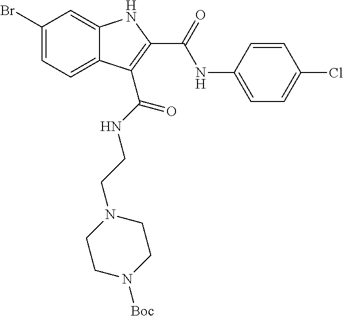 [SMILES: CC(C)(C)OC(=O)N1CCN(CCNC(=O)c2c(C(=O)Nc3ccc(Cl)cc3)[nH]c3cc(Br)ccc23)CC1] |
| 79 | 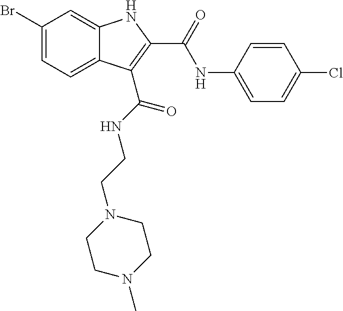 [SMILES: CN1CCN(CCNC(=O)c2c(C(=O)Nc3ccc(Cl)cc3)[nH]c3cc(Br)ccc23)CC1] |
| 80 | 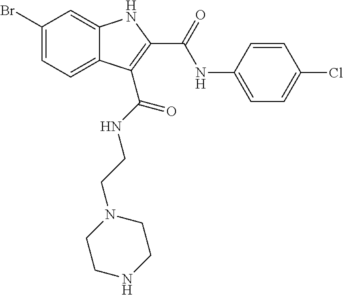 [SMILES: O=C(Nc1ccc(Cl)cc1)c1[nH]c2cc(Br)ccc2c1C(=O)NCCN1CCNCC1] |
| 81 | 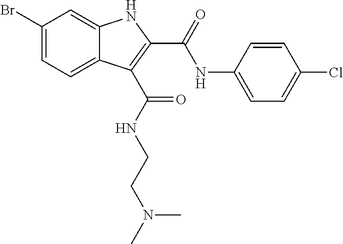 [SMILES: CN(C)CCNC(=O)c1c(C(=O)Nc2ccc(Cl)cc2)[nH]c2cc(Br)ccc12] |
| 82 | 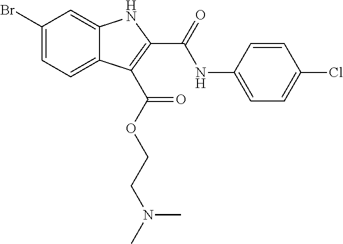 [SMILES: CN(C)CCOC(=O)c1c(C(=O)Nc2ccc(Cl)cc2)[nH]c2cc(Br)ccc12] |
| 83 | 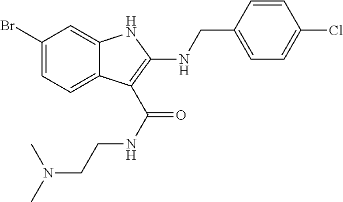 [SMILES: CN(C)CCNC(=O)c1c(NCc2ccc(Cl)cc2)[nH]c2cc(Br)ccc12] |
| 84 | 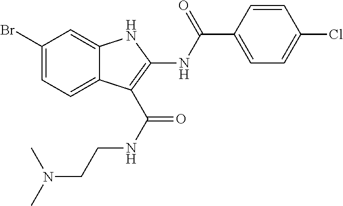 [SMILES: CN(C)CCNC(=O)c1c(NC(=O)c2ccc(Cl)cc2)[nH]c2cc(Br)ccc12] |
| 85 | 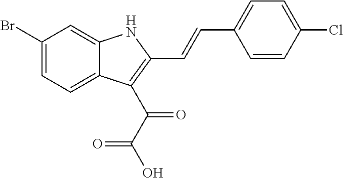 [SMILES: O=C(O)C(=O)c1c(/C=C/c2ccc(Cl)cc2)[nH]c2cc(Br)ccc12] |
| 86 | 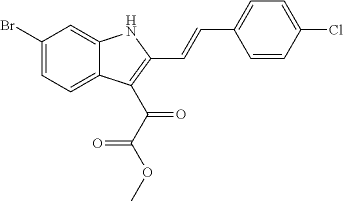 [SMILES: COC(=O)C(=O)c1c(/C=C/c2ccc(Cl)cc2)[nH]c2cc(Br)ccc12] |
| 87 | 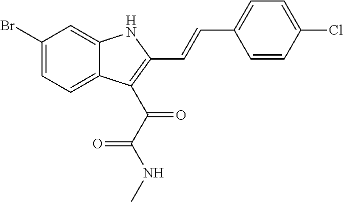 [SMILES: CNC(=O)C(=O)c1c(/C=C/c2ccc(Cl)cc2)[nH]c2cc(Br)ccc12] |
| 88 | 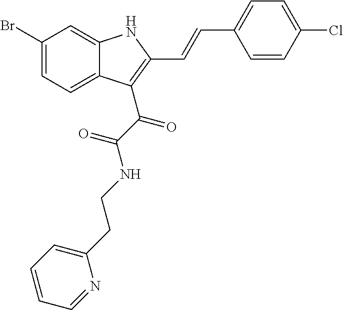 [SMILES: O=C(NCCc1ccccn1)C(=O)c1c(/C=C/c2ccc(Cl)cc2)[nH]c2cc(Br)ccc12] |
| 89 | 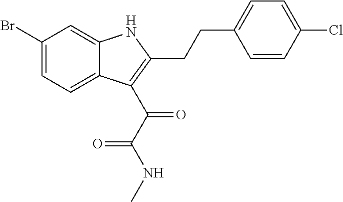 [SMILES: CNC(=O)C(=O)c1c(CCc2ccc(Cl)cc2)[nH]c2cc(Br)ccc12] |
| 90 | 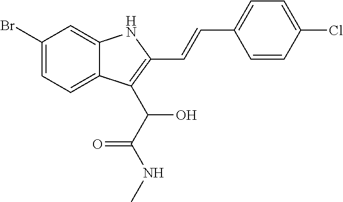 [SMILES: CNC(=O)C(O)c1c(/C=C/c2ccc(Cl)cc2)[nH]c2cc(Br)ccc12] |
| 91 | 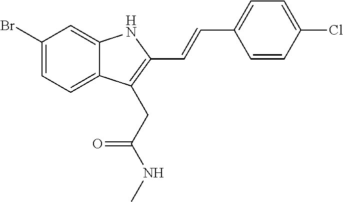 [SMILES: CNC(=O)Cc1c(/C=C/c2ccc(Cl)cc2)[nH]c2cc(Br)ccc12] |
| 92 | 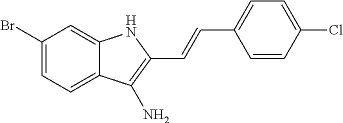 [SMILES: Nc1c(/C=C/c2ccc(Cl)cc2)[nH]c2cc(Br)ccc12] |
| 93 | 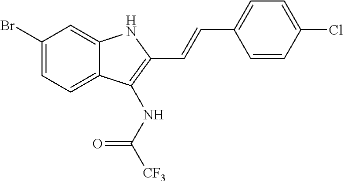 [SMILES: O=C(Nc1c(/C=C/c2ccc(Cl)cc2)[nH]c2cc(Br)ccc12)C(F)(F)F] |
| 94 | 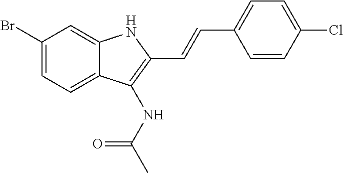 [SMILES: CC(=O)Nc1c(/C=C/c2ccc(Cl)cc2)[nH]c2cc(Br)ccc12] |
| 95 | 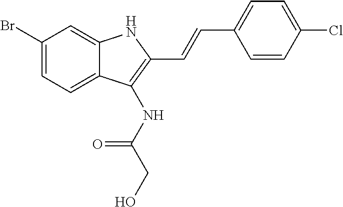 [SMILES: O=C(CO)Nc1c(/C=C/c2ccc(Cl)cc2)[nH]c2cc(Br)ccc12] |
| 96 | 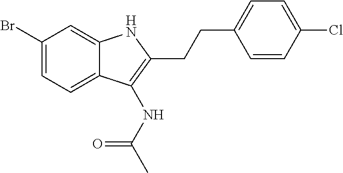 [SMILES: CC(=O)Nc1c(CCc2ccc(Cl)cc2)[nH]c2cc(Br)ccc12] |
| 97 | 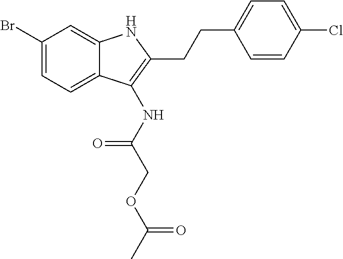 [SMILES: CC(=O)OCC(=O)Nc1c(CCc2ccc(Cl)cc2)[nH]c2cc(Br)ccc12] |
| 98 | 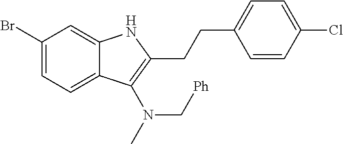 [SMILES: CN(Cc1ccccc1)c1c(CCc2ccc(Cl)cc2)[nH]c2cc(Br)ccc12] |
| 99 | 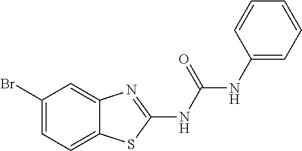 [SMILES: O=C(Nc1ccccc1)Nc1nc2cc(Br)ccc2s1] |
| 101 | 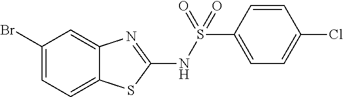 [SMILES: O=S(=O)(Nc1nc2cc(Br)ccc2s1)c1ccc(Cl)cc1] |
| 102 | 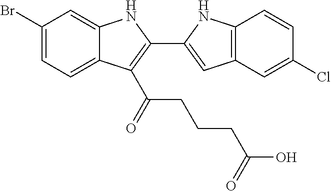 [SMILES: O=C(O)CCCC(=O)c1c(-c2cc3cc(Cl)ccc3[nH]2)[nH]c2cc(Br)ccc12] |
| 103 | 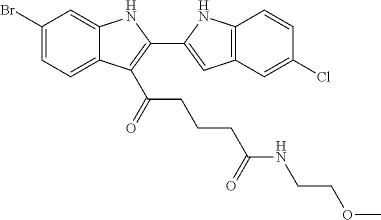 [SMILES: COCCNC(=O)CCCC(=O)c1c(-c2cc3cc(Cl)ccc3[nH]2)[nH]c2cc(Br)ccc12] |
| 104 | 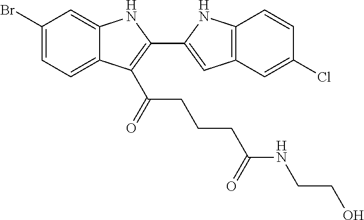 [SMILES: O=C(CCCC(=O)c1c(-c2cc3cc(Cl)ccc3[nH]2)[nH]c2cc(Br)ccc12)NCCO] |
| 105 | 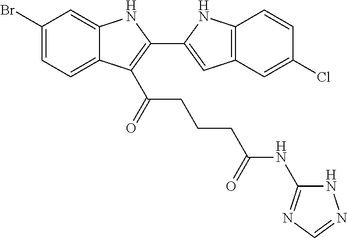 [SMILES: O=C(CCCC(=O)c1c(-c2cc3cc(Cl)ccc3[nH]2)[nH]c2cc(Br)ccc12)Nc1ncn[nH]1] |
| 106 | 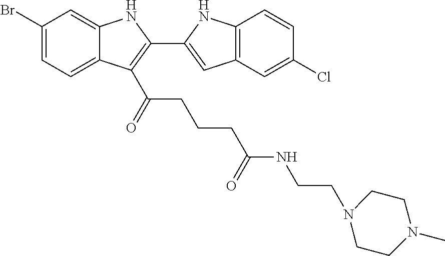 [SMILES: CN1CCN(CCNC(=O)CCCC(=O)c2c(-c3cc4cc(Cl)ccc4[nH]3)[nH]c3cc(Br)ccc23)CC1] |
| 107 | 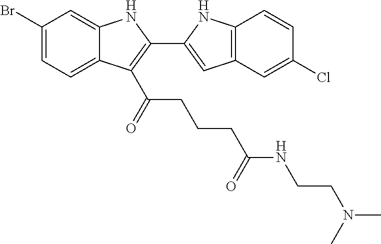 [SMILES: CN(C)CCNC(=O)CCCC(=O)c1c(-c2cc3cc(Cl)ccc3[nH]2)[nH]c2cc(Br)ccc12] |
| 108 | 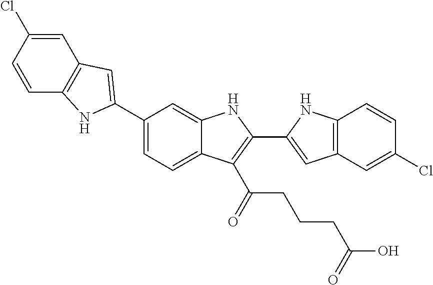 [SMILES: O=C(O)CCCC(=O)c1c(-c2cc3cc(Cl)ccc3[nH]2)[nH]c2cc(-c3cc4cc(Cl)ccc4[nH]3)ccc12] |
| 110 | 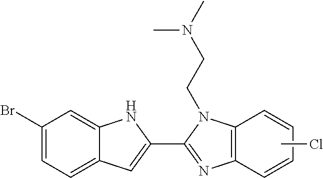 |
| 111 | 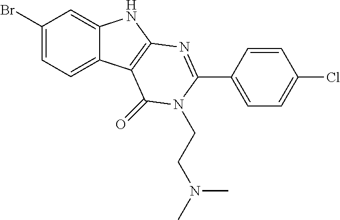 [SMILES: CN(C)CCn1c(-c2ccc(Cl)cc2)nc2[nH]c3cc(Br)ccc3c2c1=O] |
| 112 | 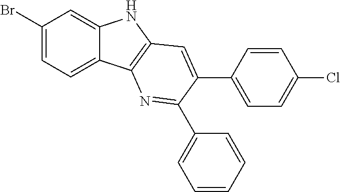 [SMILES: Clc1ccc(-c2cc3[nH]c4cc(Br)ccc4c3nc2-c2ccccc2)cc1] |
| 113 | 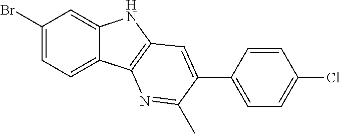 [SMILES: Cc1nc2c(cc1-c1ccc(Cl)cc1)[nH]c1cc(Br)ccc12] |
| 114 | 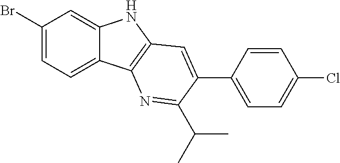 [SMILES: CC(C)c1nc2c(cc1-c1ccc(Cl)cc1)[nH]c1cc(Br)ccc12] |
| 115 | 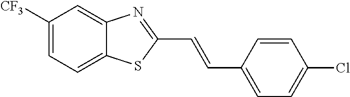 [SMILES: FC(F)(F)c1ccc2sc(/C=C/c3ccc(Cl)cc3)nc2c1] |
| 116 | 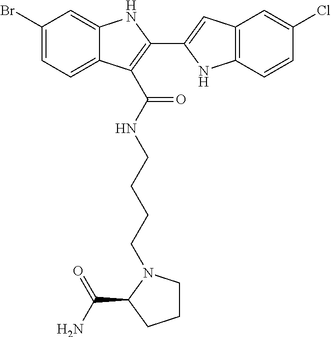 [SMILES: NC(=O)[C@@H]1CCCN1CCCCNC(=O)c1c(-c2cc3cc(Cl)ccc3[nH]2)[nH]c2cc(Br)ccc12] |
| 117 | 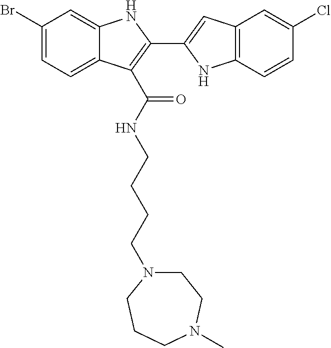 [SMILES: CN1CCCN(CCCCNC(=O)c2c(-c3cc4cc(Cl)ccc4[nH]3)[nH]c3cc(Br)ccc23)CC1] |
| 118 | 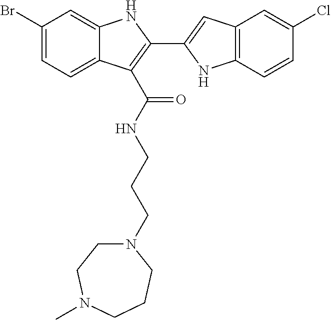 [SMILES: CN1CCCN(CCCNC(=O)c2c(-c3cc4cc(Cl)ccc4[nH]3)[nH]c3cc(Br)ccc23)CC1] |
| 119 | 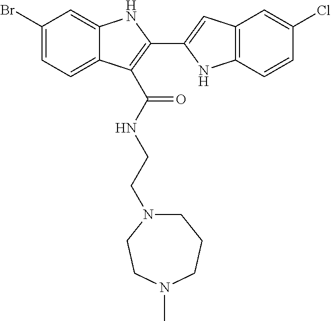 [SMILES: CN1CCCN(CCNC(=O)c2c(-c3cc4cc(Cl)ccc4[nH]3)[nH]c3cc(Br)ccc23)CC1] |
| 120 | 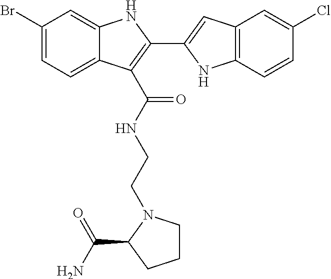 [SMILES: NC(=O)[C@@H]1CCCN1CCNC(=O)c1c(-c2cc3cc(Cl)ccc3[nH]2)[nH]c2cc(Br)ccc12] |
| 121 | 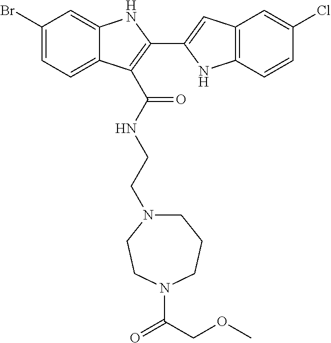 [SMILES: COCC(=O)N1CCCN(CCNC(=O)c2c(-c3cc4cc(Cl)ccc4[nH]3)[nH]c3cc(Br)ccc23)CC1] |
| 122 | 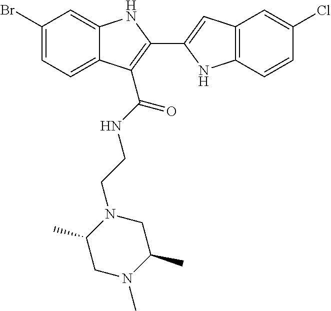 [SMILES: C[C@@H]1CN(CCNC(=O)c2c(-c3cc4cc(Cl)ccc4[nH]3)[nH]c3cc(Br)ccc23)[C@@H](C)CN1C] |
| 123 | 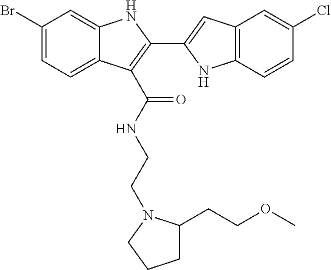 [SMILES: COCCC1CCCN1CCNC(=O)c1c(-c2cc3cc(Cl)ccc3[nH]2)[nH]c2cc(Br)ccc12] |
| 124 | 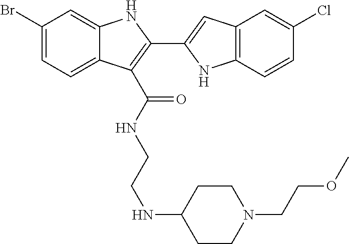 [SMILES: COCCN1CCC(NCCNC(=O)c2c(-c3cc4cc(Cl)ccc4[nH]3)[nH]c3cc(Br)ccc23)CC1] |
| 125 | 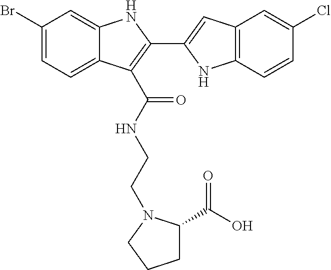 [SMILES: O=C(NCCN1CCC[C@H]1C(=O)O)c1c(-c2cc3cc(Cl)ccc3[nH]2)[nH]c2cc(Br)ccc12] |
| 126 | 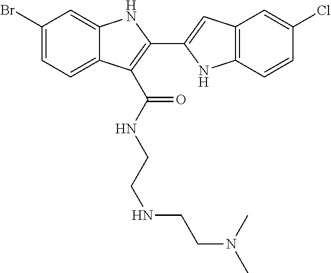 [SMILES: CN(C)CCNCCNC(=O)c1c(-c2cc3cc(Cl)ccc3[nH]2)[nH]c2cc(Br)ccc12] |
| 127 | 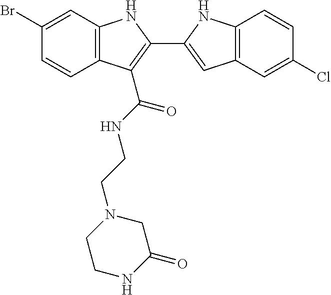 [SMILES: O=C1CN(CCNC(=O)c2c(-c3cc4cc(Cl)ccc4[nH]3)[nH]c3cc(Br)ccc23)CCN1] |
| 128 | 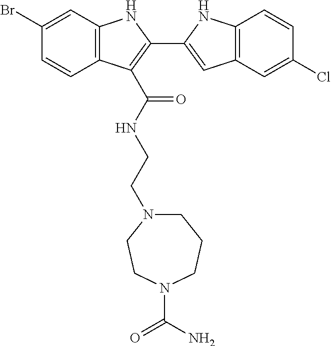 [SMILES: NC(=O)N1CCCN(CCNC(=O)c2c(-c3cc4cc(Cl)ccc4[nH]3)[nH]c3cc(Br)ccc23)CC1] |
| 129 | 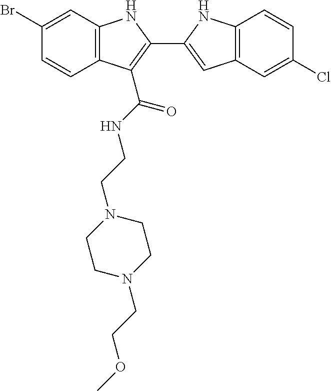 [SMILES: COCCN1CCN(CCNC(=O)c2c(-c3cc4cc(Cl)ccc4[nH]3)[nH]c3cc(Br)ccc23)CC1] |
| 130 | 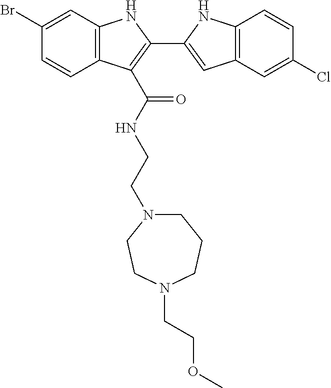 [SMILES: COCCN1CCCN(CCNC(=O)c2c(-c3cc4cc(Cl)ccc4[nH]3)[nH]c3cc(Br)ccc23)CC1] |
| 131 | 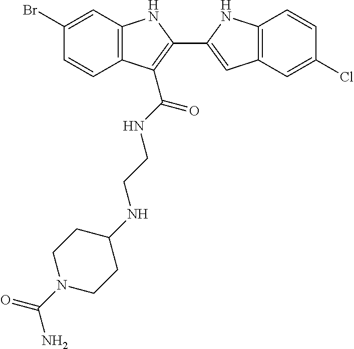 [SMILES: NC(=O)N1CCC(NCCNC(=O)c2c(-c3cc4cc(Cl)ccc4[nH]3)[nH]c3cc(Br)ccc23)CC1] |
| 132 | 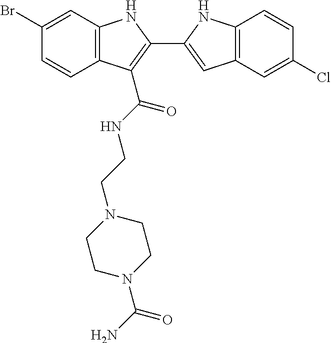 [SMILES: NC(=O)N1CCN(CCNC(=O)c2c(-c3cc4cc(Cl)ccc4[nH]3)[nH]c3cc(Br)ccc23)CC1] |
| 133 | 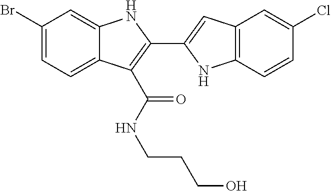 [SMILES: O=C(NCCCO)c1c(-c2cc3cc(Cl)ccc3[nH]2)[nH]c2cc(Br)ccc12] |
| 134 | 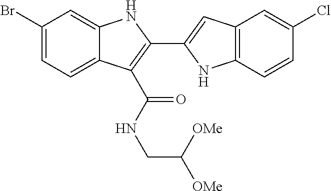 [SMILES: COC(CNC(=O)c1c(-c2cc3cc(Cl)ccc3[nH]2)[nH]c2cc(Br)ccc12)OC] |
| 135 | 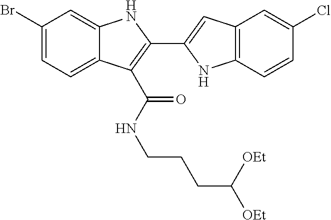 [SMILES: CCOC(CCCNC(=O)c1c(-c2cc3cc(Cl)ccc3[nH]2)[nH]c2cc(Br)ccc12)OCC] |
| 136 | 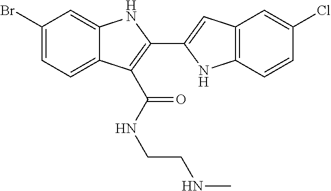 [SMILES: CNCCNC(=O)c1c(-c2cc3cc(Cl)ccc3[nH]2)[nH]c2cc(Br)ccc12] |
| 137 | 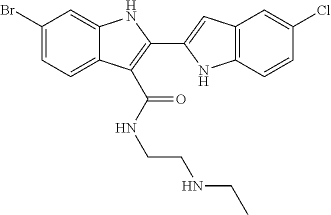 [SMILES: CCNCCNC(=O)c1c(-c2cc3cc(Cl)ccc3[nH]2)[nH]c2cc(Br)ccc12] |
| 138 | 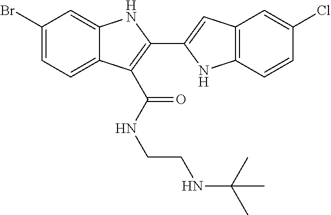 [SMILES: CC(C)(C)NCCNC(=O)c1c(-c2cc3cc(Cl)ccc3[nH]2)[nH]c2cc(Br)ccc12] |
| 139 | 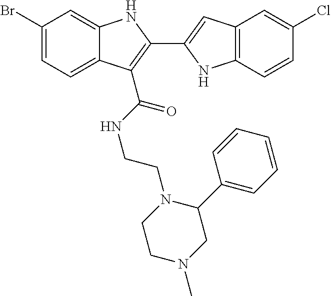 [SMILES: CN1CCN(CCNC(=O)c2c(-c3cc4cc(Cl)ccc4[nH]3)[nH]c3cc(Br)ccc23)C(c2ccccc2)C1] |
| 140 | 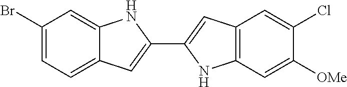 [SMILES: COc1cc2[nH]c(-c3cc4ccc(Br)cc4[nH]3)cc2cc1Cl] |
| 141 | 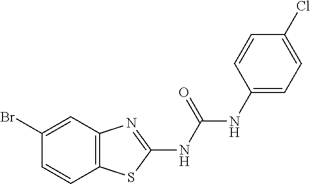 [SMILES: O=C(Nc1ccc(Cl)cc1)Nc1nc2cc(Br)ccc2s1] |
| 142 | 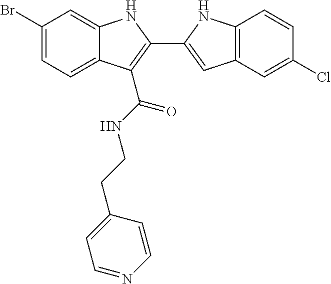 [SMILES: O=C(NCCc1ccncc1)c1c(-c2cc3cc(Cl)ccc3[nH]2)[nH]c2cc(Br)ccc12] |
| 143 | 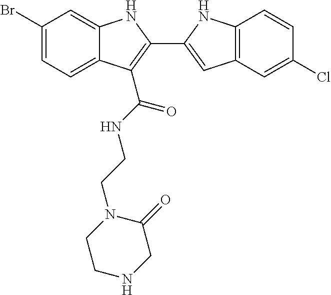 [SMILES: O=C(NCCN1CCNCC1=O)c1c(-c2cc3cc(Cl)ccc3[nH]2)[nH]c2cc(Br)ccc12] |
| 144 | 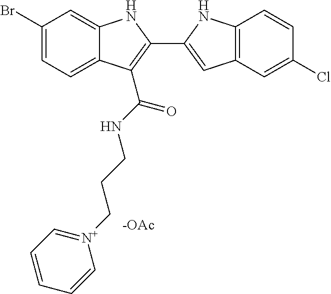 [SMILES: O=C(NCCC[n+]1ccccc1)c1c(-c2cc3cc(Cl)ccc3[nH]2)[nH]c2cc(Br)ccc12] |
| 145 | 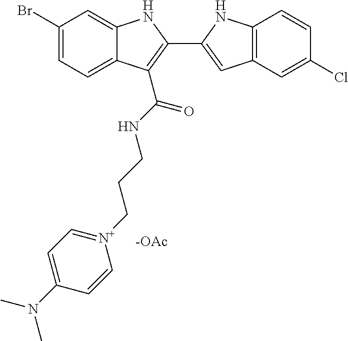 [SMILES: CN(C)c1cc[n+](CCCNC(=O)c2c(-c3cc4cc(Cl)ccc4[nH]3)[nH]c3cc(Br)ccc23)cc1] |
| 146 | 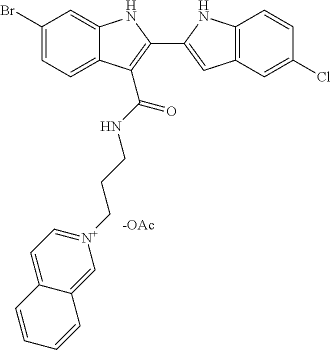 [SMILES: O=C(NCCC[n+]1ccc2ccccc2c1)c1c(-c2cc3cc(Cl)ccc3[nH]2)[nH]c2cc(Br)ccc12] |
| 147 | 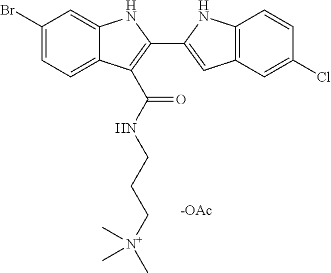 [SMILES: C[N+](C)(C)CCCNC(=O)c1c(-c2cc3cc(Cl)ccc3[nH]2)[nH]c2cc(Br)ccc12] |
| 148 | 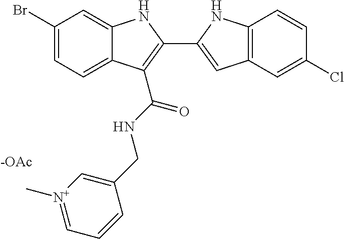 [SMILES: C[n+]1cccc(CNC(=O)c2c(-c3cc4cc(Cl)ccc4[nH]3)[nH]c3cc(Br)ccc23)c1] |
| 149 | 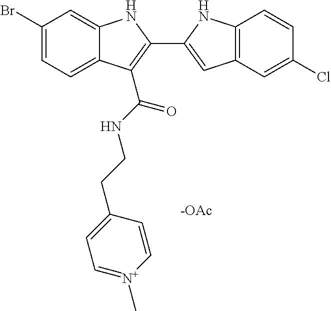 [SMILES: C[n+]1ccc(CCNC(=O)c2c(-c3cc4cc(Cl)ccc4[nH]3)[nH]c3cc(Br)ccc23)cc1] |
| 150 | 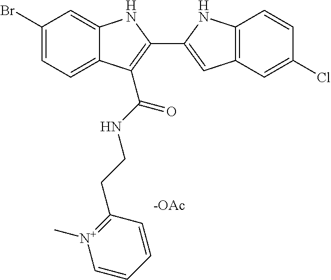 [SMILES: C[n+]1ccccc1CCNC(=O)c1c(-c2cc3cc(Cl)ccc3[nH]2)[nH]c2cc(Br)ccc12] |
| 151 | 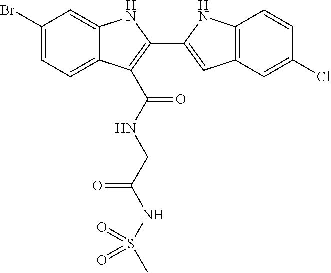 [SMILES: CS(=O)(=O)NC(=O)CNC(=O)c1c(-c2cc3cc(Cl)ccc3[nH]2)[nH]c2cc(Br)ccc12] |
| 152 | 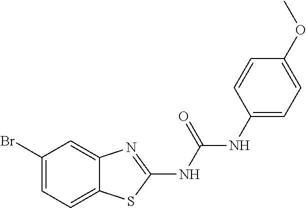 [SMILES: COc1ccc(NC(=O)Nc2nc3cc(Br)ccc3s2)cc1] |
| 153 | 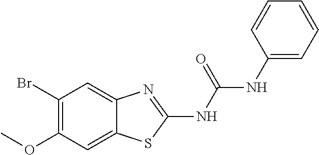 [SMILES: COc1cc2sc(NC(=O)Nc3ccccc3)nc2cc1Br] |
| 154 | 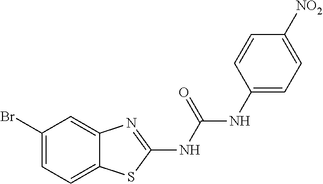 [SMILES: O=C(Nc1ccc([N+](=O)[O-])cc1)Nc1nc2cc(Br)ccc2s1] |
| 155 | 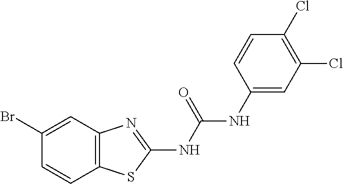 [SMILES: O=C(Nc1ccc(Cl)c(Cl)c1)Nc1nc2cc(Br)ccc2s1] |
| 156 | 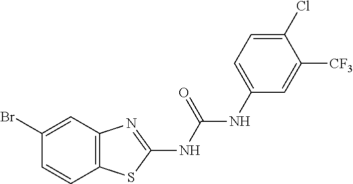 [SMILES: O=C(Nc1ccc(Cl)c(C(F)(F)F)c1)Nc1nc2cc(Br)ccc2s1] |
| 157 | 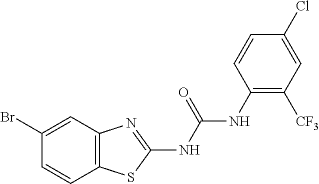 [SMILES: O=C(Nc1nc2cc(Br)ccc2s1)Nc1ccc(Cl)cc1C(F)(F)F] |
| 158 | 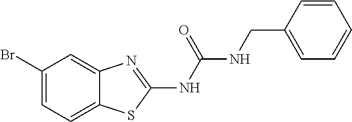 [SMILES: O=C(NCc1ccccc1)Nc1nc2cc(Br)ccc2s1] |
| 159 | 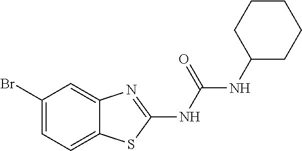 [SMILES: O=C(Nc1nc2cc(Br)ccc2s1)NC1CCCCC1] |
| 160 | 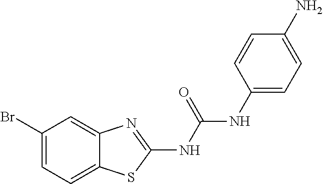 [SMILES: Nc1ccc(NC(=O)Nc2nc3cc(Br)ccc3s2)cc1] |
| 161 | 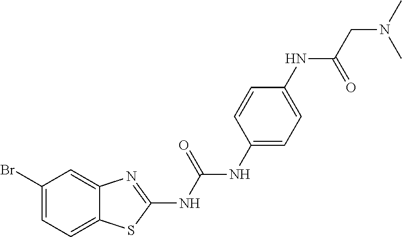 [SMILES: CN(C)CC(=O)Nc1ccc(NC(=O)Nc2nc3cc(Br)ccc3s2)cc1] |
| 163 | 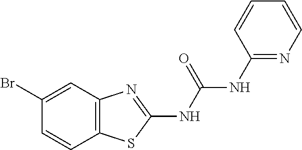 [SMILES: O=C(Nc1ccccn1)Nc1nc2cc(Br)ccc2s1] |
| 165 | 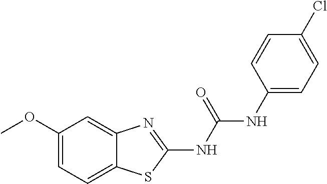 [SMILES: COc1ccc2sc(NC(=O)Nc3ccc(Cl)cc3)nc2c1] |
| 166 | 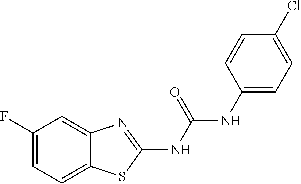 [SMILES: O=C(Nc1ccc(Cl)cc1)Nc1nc2cc(F)ccc2s1] |
| 167 | 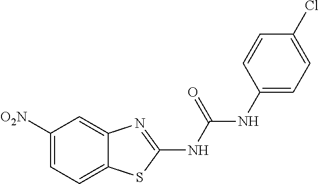 [SMILES: O=C(Nc1ccc(Cl)cc1)Nc1nc2cc([N+](=O)[O-])ccc2s1] |
| 168 | 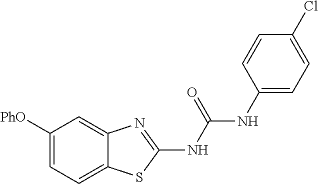 [SMILES: O=C(Nc1ccc(Cl)cc1)Nc1nc2cc(Oc3ccccc3)ccc2s1] |
| 169 | 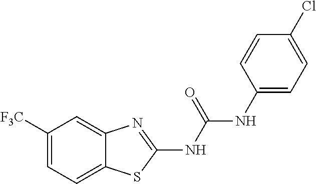 [SMILES: O=C(Nc1ccc(Cl)cc1)Nc1nc2cc(C(F)(F)F)ccc2s1] |
| 170 | 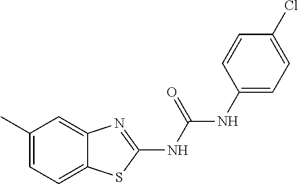 [SMILES: Cc1ccc2sc(NC(=O)Nc3ccc(Cl)cc3)nc2c1] |
| 171 | 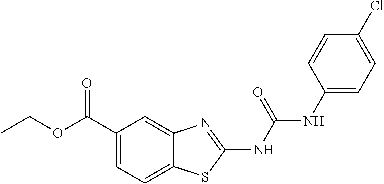 [SMILES: CCOC(=O)c1ccc2sc(NC(=O)Nc3ccc(Cl)cc3)nc2c1] |
| 172 | 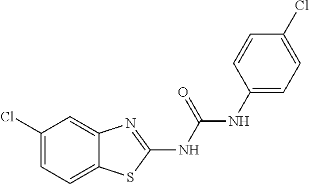 [SMILES: O=C(Nc1ccc(Cl)cc1)Nc1nc2cc(Cl)ccc2s1] |
| 173 | 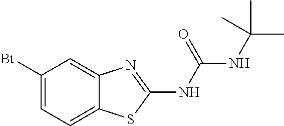 |
| 174 | 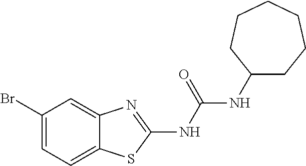 [SMILES: O=C(Nc1nc2cc(Br)ccc2s1)NC1CCCCCC1] |
| 177 | 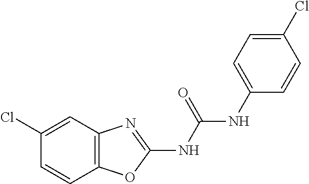 [SMILES: O=C(Nc1ccc(Cl)cc1)Nc1nc2cc(Cl)ccc2o1] |
| 179 | 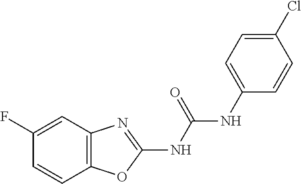 [SMILES: O=C(Nc1ccc(Cl)cc1)Nc1nc2cc(F)ccc2o1] |
| 180 | 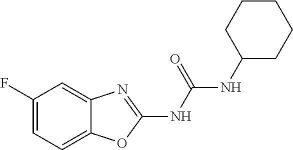 [SMILES: O=C(Nc1nc2cc(F)ccc2o1)NC1CCCCC1] |
| 181 | 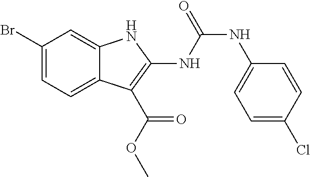 [SMILES: COC(=O)c1c(NC(=O)Nc2ccc(Cl)cc2)[nH]c2cc(Br)ccc12] |
| 182 | 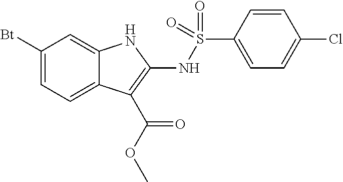 |
| 183 | 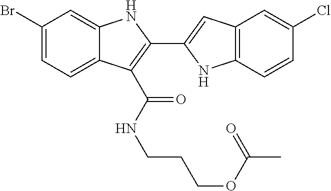 [SMILES: CC(=O)OCCCNC(=O)c1c(-c2cc3cc(Cl)ccc3[nH]2)[nH]c2cc(Br)ccc12] |
| 184 | 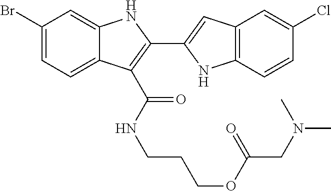 [SMILES: CN(C)CC(=O)OCCCNC(=O)c1c(-c2cc3cc(Cl)ccc3[nH]2)[nH]c2cc(Br)ccc12] |
| 185 | 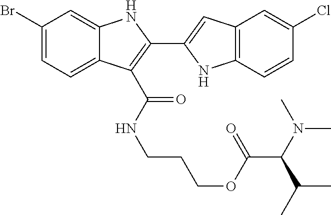 [SMILES: CC(C)[C@@H](C(=O)OCCCNC(=O)c1c(-c2cc3cc(Cl)ccc3[nH]2)[nH]c2cc(Br)ccc12)N(C)C] |
| 186 | 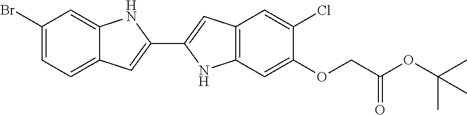 [SMILES: CC(C)(C)OC(=O)COc1cc2[nH]c(-c3cc4ccc(Br)cc4[nH]3)cc2cc1Cl] |
| 187 | 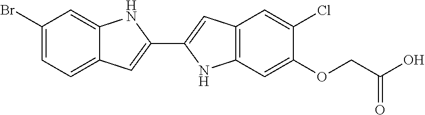 [SMILES: O=C(O)COc1cc2[nH]c(-c3cc4ccc(Br)cc4[nH]3)cc2cc1Cl] |
| 188 | 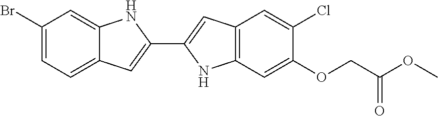 [SMILES: COC(=O)COc1cc2[nH]c(-c3cc4ccc(Br)cc4[nH]3)cc2cc1Cl] |
| 189 | 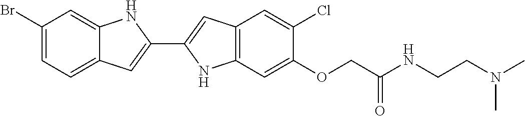 [SMILES: CN(C)CCNC(=O)COc1cc2[nH]c(-c3cc4ccc(Br)cc4[nH]3)cc2cc1Cl] |
| 190 | 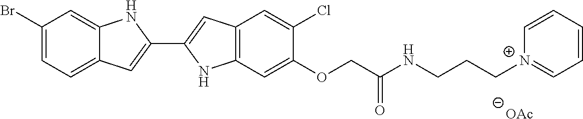 [SMILES: O=C(COc1cc2[nH]c(-c3cc4ccc(Br)cc4[nH]3)cc2cc1Cl)NCCC[n+]1ccccc1 ; "OAc"] |
| 191 | 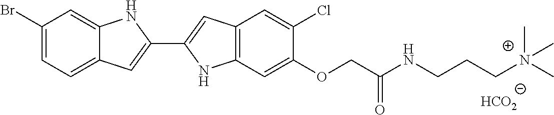 |
| 192 | 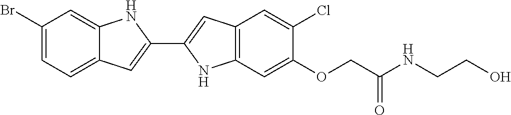 [SMILES: O=C(COc1cc2[nH]c(-c3cc4ccc(Br)cc4[nH]3)cc2cc1Cl)NCCO] |
| 193 | 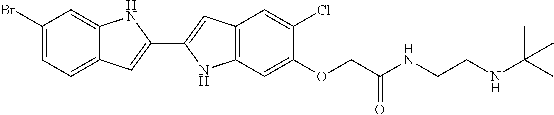 [SMILES: CC(C)(C)NCCNC(=O)COc1cc2[nH]c(-c3cc4ccc(Br)cc4[nH]3)cc2cc1Cl] |
| 194 | 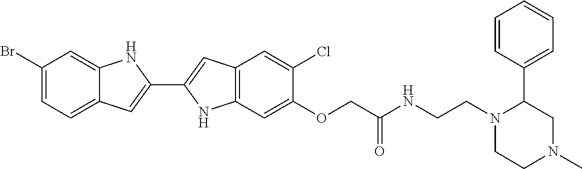 [SMILES: CN1CCN(CCNC(=O)COc2cc3[nH]c(-c4cc5ccc(Br)cc5[nH]4)cc3cc2Cl)C(c2ccccc2)C1] |
| 195 | 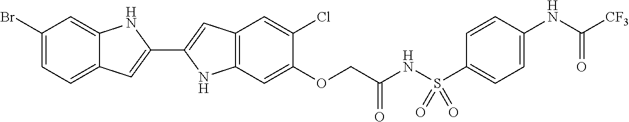 [SMILES: O=C(COc1cc2[nH]c(-c3cc4ccc(Br)cc4[nH]3)cc2cc1Cl)NS(=O)(=O)c1ccc(NC(=O)C(F)(F)F)cc1] |
| 196 | 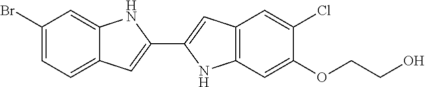 [SMILES: OCCOc1cc2[nH]c(-c3cc4ccc(Br)cc4[nH]3)cc2cc1Cl] |
| 197 | 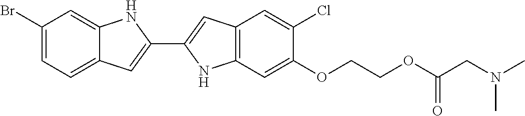 [SMILES: CN(C)CC(=O)OCCOc1cc2[nH]c(-c3cc4ccc(Br)cc4[nH]3)cc2cc1Cl] |
| 198 | 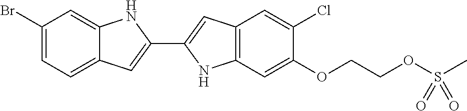 [SMILES: CS(=O)(=O)OCCOc1cc2[nH]c(-c3cc4ccc(Br)cc4[nH]3)cc2cc1Cl] |
| 199 | 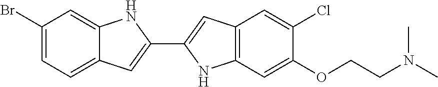 [SMILES: CN(C)CCOc1cc2[nH]c(-c3cc4ccc(Br)cc4[nH]3)cc2cc1Cl] |
| 200 | 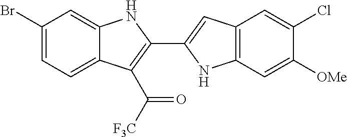 [SMILES: COc1cc2[nH]c(-c3[nH]c4cc(Br)ccc4c3C(=O)C(F)(F)F)cc2cc1Cl] |
| 201 | 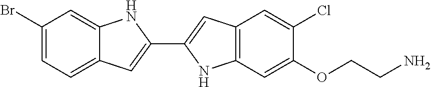 [SMILES: NCCOc1cc2[nH]c(-c3cc4ccc(Br)cc4[nH]3)cc2cc1Cl] |
| 202 | 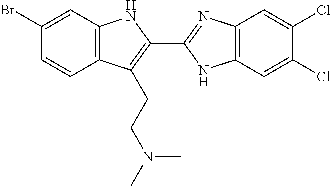 [SMILES: CN(C)CCc1c(-c2nc3cc(Cl)c(Cl)cc3[nH]2)[nH]c2cc(Br)ccc12] |
| 203 | 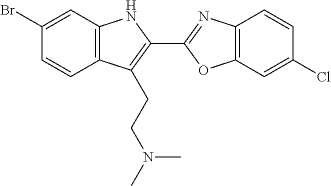 [SMILES: CN(C)CCc1c(-c2nc3ccc(Cl)cc3o2)[nH]c2cc(Br)ccc12] |
| 204 | 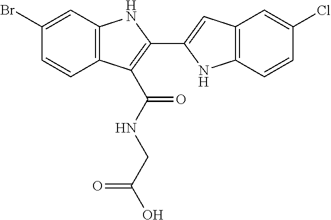 [SMILES: O=C(O)CNC(=O)c1c(-c2cc3cc(Cl)ccc3[nH]2)[nH]c2cc(Br)ccc12] |
| 205 | 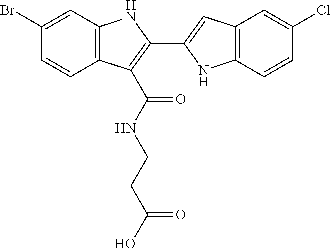 [SMILES: O=C(O)CCNC(=O)c1c(-c2cc3cc(Cl)ccc3[nH]2)[nH]c2cc(Br)ccc12] |
| 206 | 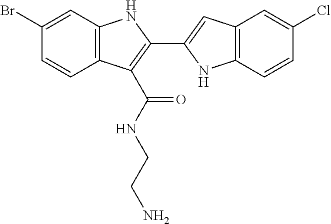 [SMILES: NCCNC(=O)c1c(-c2cc3cc(Cl)ccc3[nH]2)[nH]c2cc(Br)ccc12] |
| 207 | 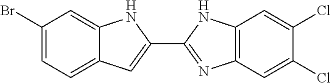 [SMILES: Clc1cc2nc(-c3cc4ccc(Br)cc4[nH]3)[nH]c2cc1Cl] |
| 208 | 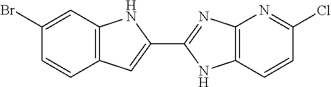 [SMILES: Clc1ccc2[nH]c(-c3cc4ccc(Br)cc4[nH]3)nc2n1] |
| 209 | 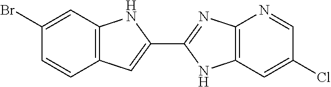 [SMILES: Clc1cnc2nc(-c3cc4ccc(Br)cc4[nH]3)[nH]c2c1] |
| 210 | 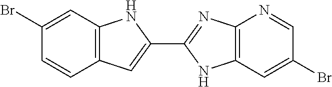 [SMILES: Brc1ccc2cc(-c3nc4ncc(Br)cc4[nH]3)[nH]c2c1] |
| 211 | 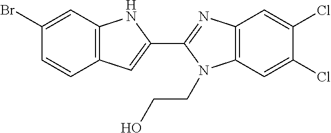 [SMILES: OCCn1c(-c2cc3ccc(Br)cc3[nH]2)nc2cc(Cl)c(Cl)cc21] |
| 212 | 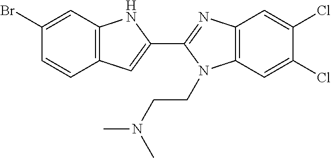 [SMILES: CN(C)CCn1c(-c2cc3ccc(Br)cc3[nH]2)nc2cc(Cl)c(Cl)cc21] |
| 213 | 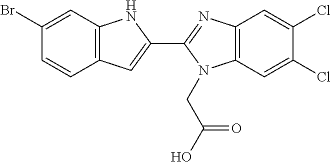 [SMILES: O=C(O)Cn1c(-c2cc3ccc(Br)cc3[nH]2)nc2cc(Cl)c(Cl)cc21] |
| 214 | 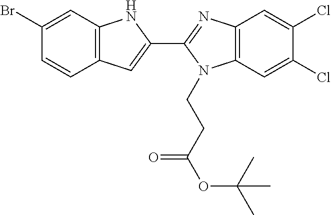 [SMILES: CC(C)(C)OC(=O)CCn1c(-c2cc3ccc(Br)cc3[nH]2)nc2cc(Cl)c(Cl)cc21] |
| 215 | 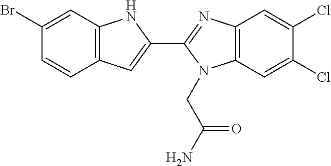 [SMILES: NC(=O)Cn1c(-c2cc3ccc(Br)cc3[nH]2)nc2cc(Cl)c(Cl)cc21] |
| 216 | 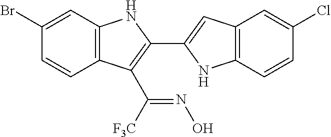 [SMILES: O/N=C(/c1c(-c2cc3cc(Cl)ccc3[nH]2)[nH]c2cc(Br)ccc12)C(F)(F)F] |
| 217 | 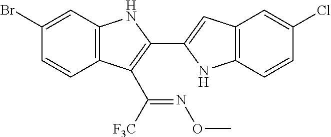 [SMILES: CO/N=C(/c1c(-c2cc3cc(Cl)ccc3[nH]2)[nH]c2cc(Br)ccc12)C(F)(F)F] |
| 218 | 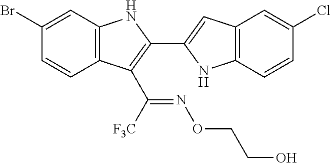 [SMILES: OCCO/N=C(/c1c(-c2cc3cc(Cl)ccc3[nH]2)[nH]c2cc(Br)ccc12)C(F)(F)F] |
| 219 | 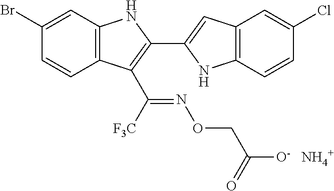 [SMILES: O=C([O-])CO/N=C(/c1c(-c2cc3cc(Cl)ccc3[nH]2)[nH]c2cc(Br)ccc12)C(F)(F)F ; [NH4+]] |
| 220 | 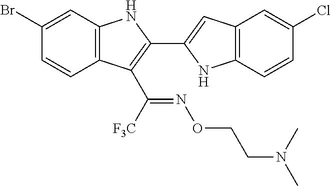 [SMILES: CN(C)CCO/N=C(/c1c(-c2cc3cc(Cl)ccc3[nH]2)[nH]c2cc(Br)ccc12)C(F)(F)F] |
| 221 | 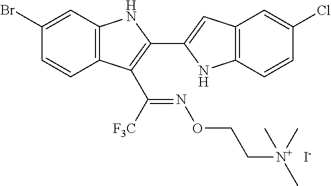 [SMILES: C[N+](C)(C)CCO/N=C(/c1c(-c2cc3cc(Cl)ccc3[nH]2)[nH]c2cc(Br)ccc12)C(F)(F)F ; [I-]] |
| 222 | 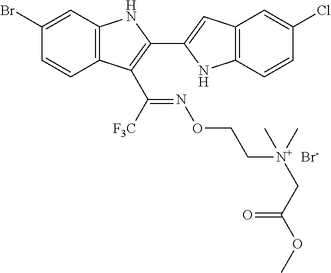 [SMILES: COC(=O)C[N+](C)(C)CCO/N=C(/c1c(-c2cc3cc(Cl)ccc3[nH]2)[nH]c2cc(Br)ccc12)C(F)(F)F ; [Br-]] |
| 223 | 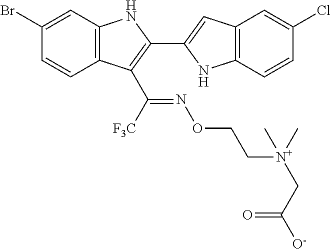 [SMILES: C[N+](C)(CCO/N=C(/c1c(-c2cc3cc(Cl)ccc3[nH]2)[nH]c2cc(Br)ccc12)C(F)(F)F)CC(=O)[O-]] |
| 224 | 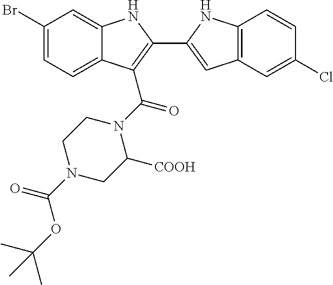 [SMILES: CC(C)(C)OC(=O)N1CCN(C(=O)c2c(-c3cc4cc(Cl)ccc4[nH]3)[nH]c3cc(Br)ccc23)C(C(=O)O)C1] |
| 225 | 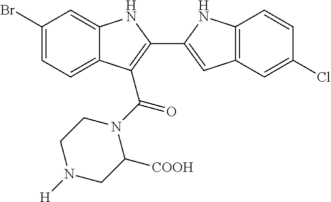 [SMILES: O=C(O)C1CNCCN1C(=O)c1c(-c2cc3cc(Cl)ccc3[nH]2)[nH]c2cc(Br)ccc12] |
| 226 | 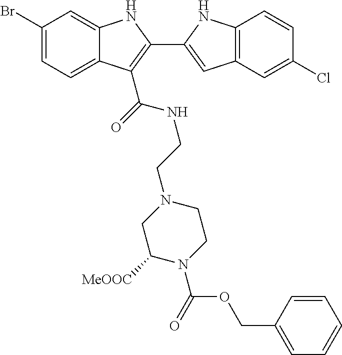 [SMILES: COC(=O)[C@@H]1CN(CCNC(=O)c2c(-c3cc4cc(Cl)ccc4[nH]3)[nH]c3cc(Br)ccc23)CCN1C(=O)OCc1ccccc1] |
| 227 | 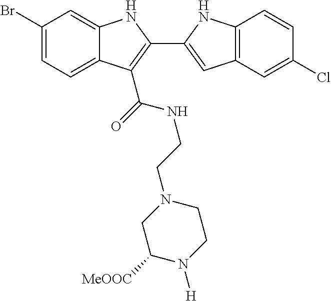 [SMILES: COC(=O)[C@@H]1CN(CCNC(=O)c2c(-c3cc4cc(Cl)ccc4[nH]3)[nH]c3cc(Br)ccc23)CCN1] |
| 228 | 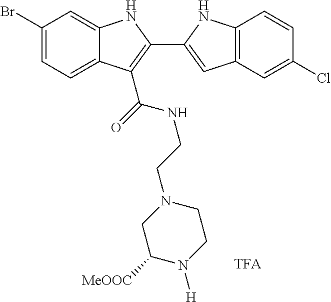 [SMILES: COC(=O)[C@@H]1CN(CCNC(=O)c2c(-c3cc4cc(Cl)ccc4[nH]3)[nH]c3cc(Br)ccc23)CCN1] |
| 229 | 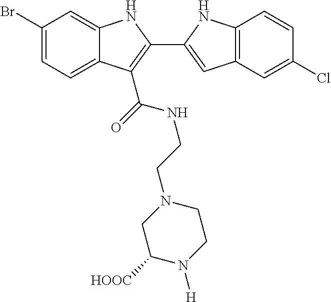 [SMILES: O=C(NCCN1CCN[C@H](C(=O)O)C1)c1c(-c2cc3cc(Cl)ccc3[nH]2)[nH]c2cc(Br)ccc12] |

or a salt thereof.

34. A method of treating a subject known to have or suspected of having a bacterial infection, the method comprising administering to the subject an effective amount of a compound having a structure of formula (1): 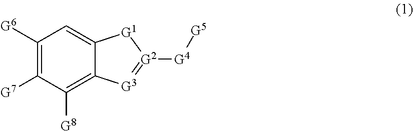
or a salt thereof,
wherein:
G¹ is NH, O, or S;
G², G³ and G⁴ may either: i) together form a ring moiety selected from the group consisting of: 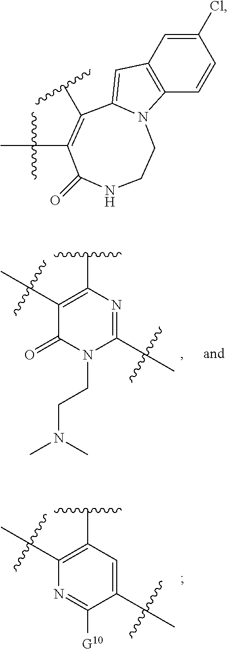
or
ii) together do not form a ring moiety wherein G² is C; G³ is N, CH or CG⁹; and
G⁴ is selected from the group consisting of: a bond, 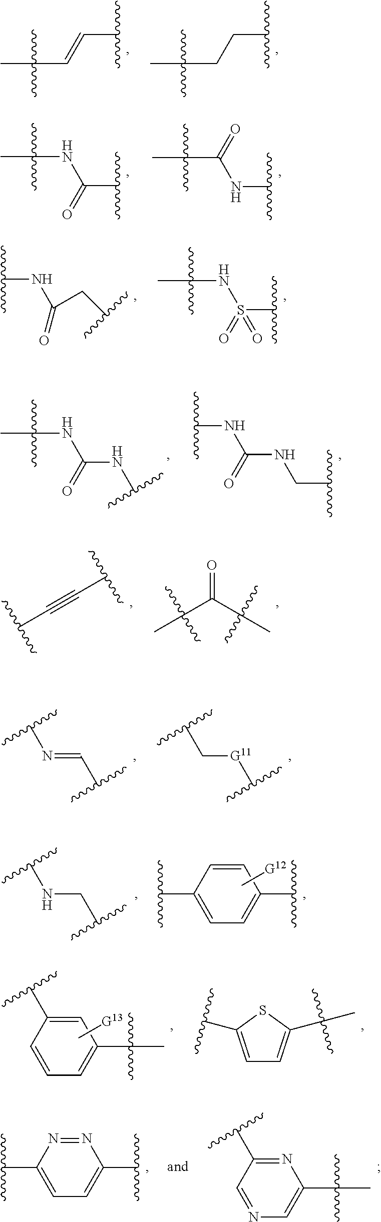
G⁵ is absent, 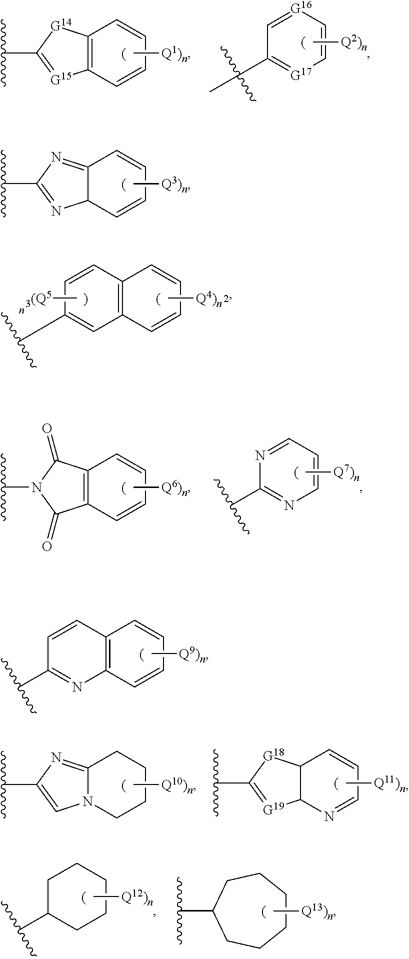
a 5-membered heteroaryl optionally substituted with (Q⁸)ₙ and containing 1 or 2 heteroatoms each heteroatom independently selected from N, O and S, substituted (C₁₋₁₁)alkyl, unsubstituted (C₁₋₁₁)alkyl, substituted (C₁₋₁₁)heteroalkyl, unsubstituted (C₁₋₁₁)heteroalkyl, substituted (C₃₋₁₁)heterocycloalkyl, unsubstituted (C₃₋₁₁)heterocycloalkyl, substituted (C₈₋₉)cycloalkyl, or unsubstituted (C₈₋₉)cycloalky;
G⁶ is H, halogen, CF₃, NO₂, substituted (C₁₋₁₁)alkyl, unsubstituted (C₁₋₁₁)alkyl, substituted (C₁₋₁₁)alkoxyl, unsubstituted (C₁₋₁₁) alkoxyl, substituted (C₆₋₁₁)aryloxy, unsubstituted (C₆₋₁₁)aryloxy, C(O)OR⁵⁰, substituted (C₁₋₁₁)heteroalkyl, unsubstituted (C₁₋₁₁) heteroalkyl or 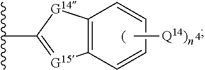
G⁷ is H, halogen, CF₃, NO₂, substituted (C₁₋₁₁)alkyl, unsubstituted (C₁₋₁₁)alkyl, substituted (C₁₋₁₁) alkoxyl, unsubstituted (C₁₋₁₁) alkoxy, substituted (C₆₋₁₁)aryloxy, unsubstituted (C₆₋₁₁)aryloxy, C(O)OR⁵¹, substituted (C₁₋₁₁)heteroalkyl, unsubstituted (C₁₋₁₁) heteroalkyl, or 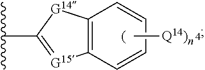
R⁵⁰ and R⁵¹ are each independently substituted (C₁₋₆)alkyl, unsubstituted (C₁₋₆)alkyl, substituted (C₁₋₆)heteroalkyl or unsubstituted (C₁₋₆) heteroalkyl;
G⁸ is H, C(═O)N(CH₃)₂, or C(═O)N(H)C(H₂)C₆H₅;
G⁹ is —CN, CF₃, —SO₂NH₂, —NH₂, —C(CF₃)₂OH, —C(CF₃)(H)OH, —C(CF₃)(CH₃)OH, —C(NOH)C(R²¹)(R²²)(R²³), C(NOH)N(R²⁴)(R²⁵), C(NOR⁶⁰)C(R⁶¹)(R⁶²)(R⁶³),
substituted (C₁₋₆) alkyl-NR⁶⁴R⁶⁵, unsubstituted (C₁₋₆) alkyl-NR⁶⁴R⁶⁵,
substituted (C₆₋₁₁) aryl, unsubstituted (C₆₋₁₁)aryl,
substituted (C₁₋₁₁) heteroaryl, unsubstituted (C₁₋₁₁) heteroaryl,
substituted (C₆₋₁₁) arylcarbonyl, unsubstituted (C₆₋₁₁) arylcarbonyl,
substituted (C₁₋₁₁) heteroarylcarbonyl, unsubstituted (C₁₋₁₁) heteroarylcarbonyl,
—CO-substituted-carbocycle, —CO-unsubstituted-carbocycle,
—CO-substituted-heterocarbocycle, —CO-unsubstituted-heterocarbocycle,
—CO-substituted-C(₁₋₆)alkyl-OR¹, —CO-unsubstituted-C(₁₋₆)alkyl-OR¹,
—CO-substituted-C(₁₋₆)alkyl-NR²R³, —CO-unsubstituted-C(₁₋₆)alkyl-NR²R³,
—CO-substituted-C(₁₋₆)alkyl-C(O)OR⁴, —CO-unsubstituted-C(₁₋₆)alkyl-C(O)OR⁴,
—CO-substituted-C(₁₋₆)alkyl-C(O)NR⁵R⁶, —CO-unsubstituted-C(₁₋₆)alkyl-C(O)NR⁵R⁶,
—C(O)NR⁷R⁸, —C(O)OR⁹, —C(O)C(O)OR¹², —C(O)C(O)NR¹³R¹⁴, —NR¹⁵R¹⁶,
—N(H)C(O)substituted-C(₁₋₆)alkyl, —N(H)C(O)unsubstituted-C(₁₋₆)alkyl,
—N(H)C(O)substituted-C(₁₋₆)haloalkyl, —N(H)C(O)unsubstituted-C(₁₋₆)haloalkyl,
—N(H)C(O)substituted-C(₆₋₁₁)aryl, —N(H)C(O)unsubstituted-C(₆₋₁₁)aryl,
—N(H)C(O)substituted-C(₁₋₁₁)heteroaryl, —N(H)C(O)unsubstituted-C(₁₋₁₁)heteroaryl,
—N(H)C(O)NR¹⁷R¹⁸,
—N(H)CO-substituted-C(₁₋₆)alkyl-OR¹⁹, —N(H)CO-unsubstituted-C(₁₋₆)alkyl-OR¹⁹,
each of R¹, R², R³, R⁴, R⁵, R⁶, R¹², R¹³, R¹⁴, R¹⁵, R¹⁶, R¹⁷, R¹⁸, R¹⁹, R²⁴, and R²⁵ is independently selected from the group consisting of: H, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl,
each of R²¹, R²², R²³, R⁶¹, R⁶² and R⁶³ is independently selected from the group consisting of: H, F, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl;
each of R⁶⁴ and R⁶⁵ is independently selected from the group consisting of: H, substituted C(₃₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl
each pair: a) R² and R³, b) R⁵ and R⁶, c) R¹³ and R¹⁴, d) R¹⁵ and R¹⁶, e) R¹⁷ and R¹⁸, and f) R⁶⁴ and R⁶⁵ may alternately be and independently as a pair be a 3-7 membered substituted heterocarbocyclic ring or a 3-7 membered unsubstituted heterocarbocyclic ring;
R⁶⁰ is unsubstituted C(₁₋₁₁)alkyl, substituted C(₁₋₁₁)alkyl, unsubstituted C(₁₋₁₁)alkyl-NR⁶⁶R⁶⁷, substituted C(₁₋₁₁)alkyl-NR⁶⁶R⁶⁷, unsubstituted C(₁₋₁₁)alkyl-N⁺R⁶⁸R⁶⁹R⁷⁰, or substituted C(₁₋₁₁)alkyl-N⁺R⁶⁸R⁶⁹R⁷⁰, wherein R⁶⁶ and R⁶⁷ are each independently H, unsubstituted C(₁₋₁₁)alkyl or substituted C(₁₋₁₁)alkyl, and R⁶⁸, R⁶⁹ and R⁷⁰ are each independently unsubstituted C(₁₋₁₁)alkyl, or substituted C(₁₋₁₁)alkyl,
each of R⁷ and R⁸ are either
I) independently selected from the group consisting of: H, substituted C(₁₋₆)alkyl, substituted C(₁₋₆)alkyl-NR⁵²R⁵³, unsubstituted C(₁₋₆)alkyl-NR⁵²R⁵³, substituted C(₁₋₆)alkyl-N⁺R⁷¹R⁷²R⁷³, unsubstituted C(₁₋₆)alkyl-N⁺R⁷¹R⁷²R⁷³, substituted C(₁₋₆)alkyl-OC(O)unsubstituted C(₁₋₆)alkyl-NR⁷⁴R⁷⁵, unsubstituted C(₁₋₆)alkyl-OC(O)unsubstituted C(₁₋₆)alkyl-NR⁷⁴R⁷⁵, substituted C(₁₋₆)alkyl-C(O)NHS(O)₂R⁷⁶, unsubstituted C(₁₋₆)alkyl-C(O)NHS(O)₂R⁷⁶, substituted C(₆₋₁₁)aryl, substituted C(₃₋₁₁)carbocyclic, substituted C(₄₋₇)heterocarbocycle, substituted C(₄₋₇)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₃₋₁₁)carbocyclic, unsubstituted C(₁₋₁₁)heterocarbocycle, unsubstituted C(₁₋₁₁)heteroaryl, unsubstituted C(₇₋₁₁)aralkyl, and unsubstituted C(₂₋₁₁)heteroaralkyl wherein each of R⁵², R⁵³, R⁷⁴ and R⁷⁵ is selected from the group consisting of: H, unsubstituted C₍₁₋₆₎alkyl, substituted C₍₃₋₇₎heterocycloalkyl, unsubstituted C₍₃₋₇₎heterocycloalkyl, substituted C₍₁₋₆₎alkyl, substituted C₍₃₋₇₎cycloalkyl and unsubstituted C₍₃₋₇₎cycloalkyl, or each pair: a) R⁵² and R⁵³, or (b) R⁷⁴ and R⁷⁵, together form a 3-7 membered substituted heterocarbocyclic ring or a 3-7 membered unsubstituted heterocarbocyclic ring, and wherein each of R⁷¹, R⁷², R⁷³ and R⁷⁶ is independently unsubstituted C(₁₋₁₁)alkyl, or substituted C(₁₋₁₁)alkyl, or
II) together form a 3-7 membered substituted heterocarbocyclic ring or a 3-7 membered unsubstituted heterocarbocyclic ring;
R⁹ is selected from the group consisting of substituted C(₁₋₆)alkyl, substituted C(₁₋₆)alkyl-NR¹⁰R¹¹, unsubstituted C(₁₋₆)alkyl-NR¹⁰R¹¹, substituted C(₁₋₆)alkyl-OR²⁰, unsubstituted C(₁₋₆)alkyl-OR²⁰, and unsubstituted C(₁₋₆)alkyl wherein each of R¹⁰, R¹¹, and R²⁰ is independently selected from the group consisting of: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; R¹⁰ and R¹¹ may alternately as a pair be a 3-7 membered substituted heterocarbocyclic ring or a 3-7 membered unsubstituted heterocarbocyclic ring, or
G⁹ is 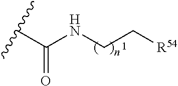
wherein n¹ is 1, 2, 3 or 4 and R⁵⁴ is 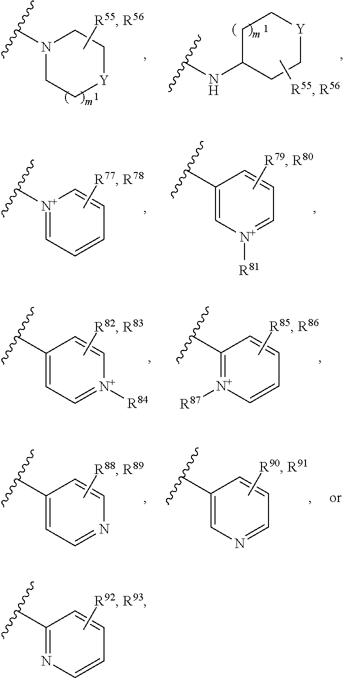
wherein m¹=0, 1 or 2, R⁵⁵ and R⁵⁶ are independently H, carbonyl (═O), Me, Ph, CO₂R⁹⁴, CO₂NH₂, C(₁₋₆)substituted alkyl or C(₁₋₆)unsubstituted alkyl, wherein R⁹⁴ is H, C(₁₋₆)unsubstituted alkyl or C(₁₋₆)substituted alkyl;
R⁷⁷, R⁷⁸, R⁷⁹, R⁸⁰, R⁸², R⁸³, R⁸⁵, R⁸⁶, R⁸⁸, R⁸⁹, R⁹⁰, R⁹¹, R⁹² and R⁹³ are each independently H, C(₁₋₆)substituted alkyl, C(₁₋₆)unsubstituted alkyl, substituted C₍₁₋₆₎heteroalkyl, unsubstituted C₍₁₋₆₎ heteroalkyl, OR⁹⁵, C(O)R⁹⁶, or NR⁹⁷R⁹⁸, wherein R⁹⁵ is H, C(₁₋₆)substituted alkyl, or C(₁₋₆)unsubstituted alkyl, R⁹⁶ is C(₁₋₆)substituted alkyl, or C(₁₋₆)unsubstituted alkyl, and R⁹⁷ and R⁹⁸ are each independently H, C(₁₋₆)substituted alkyl, or C(₁₋₆)unsubstituted alkyl, or each pair: a) R⁷⁷ and R⁷⁸, b) R⁷⁹ and R⁸⁰, c) R⁸² and R⁸³, d) R⁸⁵ and R⁸⁶, e) R⁸⁸ and R⁸⁹, f) R⁹⁰ and R⁹¹, or g) R⁹² and R⁹³ are attached to adjacent ring-forming C atoms, and together with the ring-forming C atoms, form a substituted C₆ aryl ring or an unsubstituted C₆ aryl ring;
R⁸¹, R⁸⁴ and R⁸⁷ each independently is C(₁₋₆)substituted alkyl, or C(₁₋₆)unsubstituted alkyl; and
Y is CH₂, CHOH, CHO—CO—C(₁₋₆)unsubstituted alkyl, CHO—CO—C(₁₋₆)substituted alkyl, NCONH₂, N—C(₁₋₆)substituted alkyl, N—C(₁₋₆)unsubstituted alkyl, NH or N—C(O)OR⁹⁹, wherein R⁹⁹ is C(₁₋₆)unsubstituted alkyl, C(₁₋₆)substituted alkyl, C(₆₋₁₁)unsubstituted aralkyl or C(₆₋₁₁)substituted aralkyl;
G¹⁰ is selected from the group consisting of: a straight C(₁₋₆)alkyl, a branched C(₃₋₆)alkyl and phenyl;
G¹¹ is NHCH₂, NH, NHCO, SCH₂, O, or S;
G¹² is H, NO₂, or OMe;
G¹³ is H, NO₂, or OMe;
each of G¹⁴, G^{14′} and G¹⁸ is independently NH, S, O, N—CH₃, N—CH₂—OCH₃, N—CH₂—COOH, N—CH₂—CH₂OH, N—CH₂—C(O)NH₂, CH—CH₃, N—R^{14′}, CH—R^{14′} or substituted C(₁₋₆)alkyl-NR⁵²R⁵³, wherein R^{14′} is C₍₁₋₆₎ substituted alkyl, C₍₁₋₆₎ unsubstituted alkyl, 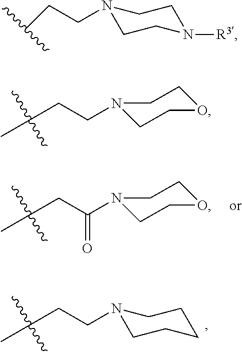
wherein R^{3′} is H, unsubstituted alkyl, or substituted alkyl, wherein the alkyl is 1-6, carbons in length, and the alkyl is optionally substituted with Br, F, Cl, I, OH, OMe, or N₃;
each of G¹⁵, G^{15′} and G¹⁹ is independently N, CH or CG⁹,
G¹⁶ is N or CH;
G¹⁷ is N or CH;
each of n, n², n³ and n⁴ is independently 0, 1, 2, 3, or 4;
each Q¹ and Q¹⁴ is independently selected from the group consisting of: halogen, —OR²⁶, —O—(C₁₋₆)alkyl-NR²⁷R²⁸, —O—(C₁₋₆)alkyl-C(O)OR¹⁰⁰, —O—(C₁₋₆)alkyl-C(O)NHR¹⁰¹, —O—(C₁₋₆)alkyl-OC(O)R¹⁰², —O—(C₁₋₆)alkyl-OS(O)₂R¹⁰³, NO₂, NR¹⁰⁴R¹⁰⁵, —NHC(O)R¹⁰⁶, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q² is independently selected from the group consisting of: halogen, —OR²⁹, —O—(C₁₋₆)alkyl-NR³⁰R³¹, —O—(C₁₋₆)alkyl-C(O)OR¹⁰⁷, —O—(C₁₋₆)alkyl-C(O)NHR¹⁰⁸, —O—(C₁₋₆)alkyl-OC(O)R¹⁰⁹, —O—(C₁₋₆)alkyl-OS(O)₂R¹¹⁰, NO₂, NR¹¹¹R¹¹², —NHC(O)R¹¹³, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q³ is independently selected from the group consisting of: halogen, —OR¹¹⁴, —O—(C₁₋₆)alkyl-NR¹¹⁵R¹¹⁶, —O—(C₁₋₆)alkyl-C(O)OR¹¹⁷, —O—(C₁₋₆)alkyl-C(O)NHR¹¹⁸, —O—(C₁₋₆)alkyl-OC(O)R¹¹⁹, —O—(C₁₋₆)alkyl-OS(O)₂R¹²⁰, NO₂, NR¹²¹R¹²², —NHC(O)R¹²³, substituted C(₁₋₆)alkyl, substituted C(₁₋₆)heteroalkyl, unsubstituted C(₁₋₆)alkyl, and unsubstituted C(₁₋₆)heteroalkyl;
each Q⁴ is independently selected from the group consisting of: halogen, —OR³⁵, —O—(C₁₋₆)alkyl-NR³⁶R³⁷, —O—(C₁₋₆)alkyl-C(O)OR¹²⁴, —O—(C₁₋₆)alkyl-C(O)NHR¹²⁵, —O—(C₁₋₆)alkyl-OC(O)R¹²⁶, —O—(C₁₋₆)alkyl-OS(O)₂R¹²⁷, NO₂, NR¹²⁸R¹²⁹, —NHC(O)R¹³⁰, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q⁵ is independently selected from the group consisting of: halogen, —OR³⁸, —O—(C₁₋₆)alkyl-NR³⁹R⁴⁰, —O—(C₁₋₆)alkyl-C(O)OR¹³¹, —O—(C₁₋₆)alkyl-C(O)NHR¹³², —O—(C₁₋₆)alkyl-OC(O)R¹³³, —O—(C₁₋₆)alkyl-OS(O)₂R¹³⁴, NO₂, NR¹³⁵R¹³⁶, —NHC(O)R¹³⁷, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q⁶ is independently selected from the group consisting of: halogen, —OR⁴¹, —O—(C₁₋₆)alkyl-NR⁴²R⁴³, —O—(C₁₋₆)alkyl-C(O)OR¹³⁸, —O—(C₁₋₆)alkyl-C(O)NHR¹³⁹, —O—(C₁₋₆)alkyl-OC(O)R¹⁴⁰, —O—(C₁₋₆)alkyl-OS(O)₂R¹⁴¹, NO₂, NR¹⁴²R¹⁴³, —NHC(O)R¹⁴⁴, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q⁷ is independently selected from the group consisting of: halogen, —OR⁴⁴, —O—(C₁₋₆)alkyl-NR⁴⁵R⁴⁶, —O—(C₁₋₆)alkyl-C(O)OR¹⁴⁵, —O—(C₁₋₆)alkyl-C(O)NHR¹⁴⁶, —O—(C₁₋₆)alkyl-OC(O)R¹⁴⁷, —O—(C₁₋₆)alkyl-OS(O)₂R¹⁴⁸, NO₂, NR¹⁴⁹R¹⁵⁰, —NHC(O)R¹⁵¹, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q⁸ is independently selected from the group consisting of: halogen, —OR⁴⁷, —O—(C₁₋₆)alkyl-NR⁴⁸R⁴⁹, —O—(C₁₋₆)alkyl-C(O)OR¹⁵², —O—(C₁₋₆)alkyl-C(O)NHR¹⁵³, —O—(C₁₋₆)alkyl-OC(O)R¹⁵⁴, —O—(C₁₋₆)alkyl-OS(O)₂R¹⁵⁵, NO₂, NR¹⁵⁶R¹⁵⁷, —NHC(O)R¹⁵⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q⁹ is independently selected from the group consisting of: halogen, —OR¹⁵⁹, —O—(C₁₋₆)alkyl-NR¹⁶⁰R¹⁶¹, —O—(C₁₋₆)alkyl-C(O)OR¹⁶², —O—(C₁₋₆)alkyl-C(O)NHR¹⁶³, —O—(C₁₋₆)alkyl-OC(O)R¹⁶⁴, —O—(C₁₋₆)alkyl-OS(O)₂R¹⁶⁵, NO₂, NR¹⁶⁶R¹⁶⁷, —NHC(O)R¹⁶⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q¹⁰ is independently selected from the group consisting of:
halogen, —OR¹⁶⁹, —O—(C₁₋₆)alkyl-NR¹⁷⁰R¹⁷¹, —O—(C₁₋₆)alkyl-C(O)OR¹⁷², —O—(C₁₋₆)alkyl-C(O)NHR¹⁷³, —O—(C₁₋₆)alkyl-OC(O)R¹⁷⁴, —O—(C₁₋₆)alkyl-OS(O)₂R¹⁷⁵, NO₂, NR¹⁷⁶R¹⁷⁷, —NHC(O)R¹⁷⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q¹¹ is independently selected from the group consisting of:
halogen, —OR¹⁷⁹, —O—(C₁₋₆)alkyl-NR¹⁸⁰R¹⁸¹, —O—(C₁₋₆)alkyl-C(O)OR¹⁸², —O—(C₁₋₆)alkyl-C(O)NHR¹⁸³, —O—(C₁₋₆)alkyl-OC(O)R¹⁸⁴, —O—(C₁₋₆)alkyl-OS(O)₂R¹⁸⁵, NO₂, NR¹⁸⁶R¹⁸⁷, —NHC(O)R¹⁸⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q¹² is independently selected from the group consisting of:
halogen, —OR¹⁸⁹, —O—(C₁₋₆)alkyl-NR¹⁹⁰R¹⁹¹, —O—(C₁₋₆)alkyl-C(O)OR¹⁹², —O—(C₁₋₆)alkyl-C(O)NHR¹⁹³, —O—(C₁₋₆)alkyl-OC(O)R¹⁹⁴, —O—(C₁₋₆)alkyl-OS(O)₂R¹⁹⁵, NO₂, NR¹⁹⁶R¹⁹⁷, —NHC(O)R¹⁹⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q¹³ is independently selected from the group consisting of:
halogen, —OR¹⁹⁹, —O—(C₁₋₆)alkyl-NR²⁰⁰R²⁰¹, —O—(C₁₋₆)alkyl-C(O)OR²⁰², —O—(C₁₋₆)alkyl-C(O)NHR²⁰³, —O—(C₁₋₆)alkyl-OC(O)R²⁰⁴, —O—(C₁₋₆)alkyl-OS(O)₂R²⁰⁵, NO₂, NR²⁰⁶R²⁰⁷, —NHC(O)R²⁰⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each R²⁶, R²⁷, R²⁸, R²⁹, R³⁰, R³¹, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R¹⁰⁰, R¹⁰⁴, R¹⁰⁵, R¹⁰⁷, R¹¹¹, R¹¹², R¹¹⁴, R¹¹⁵, R¹¹⁶, R¹¹⁷, R¹²¹, R¹²², R¹²⁴, R¹²⁸, R¹²⁹, R¹³¹, R¹³⁵, R¹³⁶, R¹³⁸, R¹⁴², R¹⁴³, R¹⁴⁵, R¹⁴⁹, R¹⁵⁰, R¹⁵², R¹⁵⁶, R¹⁵⁷, R¹⁵⁹, R¹⁶⁰, R¹⁶¹, R¹⁶², R¹⁶⁶, R¹⁶⁷, R¹⁶⁹, R¹⁷⁰, R¹⁷¹, R¹⁷², R¹⁷⁶, R¹⁷⁷, R¹⁷⁹, R¹⁸⁰, R¹⁸¹, R¹⁸², R¹⁸⁶, R¹⁸⁷, R¹⁸⁹, R¹⁹⁰, R¹⁹¹, R¹⁹², R¹⁹⁶, R¹⁹⁷, R¹⁹⁹, R²⁰⁰, R²⁰¹, R²⁰², R²⁰⁶ and R²⁰⁷ are independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and each pair: a) R²⁷ and R²⁸, b) R³⁰ and R³¹, c) R³⁶ and R³⁷, d) R³⁹ and R⁴⁰, e) R⁴² and R⁴³, f) R⁴⁵ and R⁴⁶, g) R⁴⁸ and R⁴⁹, h) R¹⁰⁴ and R¹⁰⁵, i) R¹¹¹ and R¹¹², j) R¹¹⁵ and R¹¹⁶, k) R¹²¹ and R¹²², l) R¹²⁸ and R¹²⁹, m) R¹³⁵ and R¹³⁶, n) R¹⁴² and R¹⁴³, o) R¹⁴⁹ and R¹⁵⁰, p) R¹⁵⁶ and R¹⁵⁷, q) R¹⁶⁰ and R¹⁶¹, r) R¹⁶⁶ and R¹⁶⁷, s) R¹⁷⁰ and R¹⁷¹, t) R¹⁷⁶ and R¹⁷⁷, u) R¹⁸⁰ and R¹⁸¹, v) R¹⁸⁶ and R¹⁸⁷, w) R¹⁹⁰ and R¹⁹¹, x) R¹⁹⁶ and R¹⁹⁷, y) R²⁰⁰ and R²⁰¹, and z) R²⁰⁶ and R²⁰⁷ may alternately be and independently as a pair be a 3-7 membered substituted heterocarbocyclic ring or a 3-7 membered unsubstituted heterocarbocyclic ring;
R¹⁰¹, R¹⁰⁸, R¹¹⁸, R¹²⁵, R¹³², R¹³⁹, R¹⁴⁶, R¹⁵³, R¹⁶³, R¹⁷³, R¹⁸³, R¹⁹³ and R²⁰³, are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, unsubstituted C(₂₋₁₁)heteroaralkyl, substituted C(₁₋₆)alkyl-NR²⁰⁹R²¹⁰, unsubstituted C(₁₋₆)alkyl-NR²⁹R²¹⁰, substituted C(₁₋₆)alkyl-N⁺R²¹¹R²¹²R²¹³, unsubstituted C(₁₋₆)alkyl-N⁺R²¹¹R²¹²R²¹³, substituted C(₁₋₆)alkyl-OR²¹⁴, unsubstituted C(₁₋₆)alkyl-OR²¹⁴, 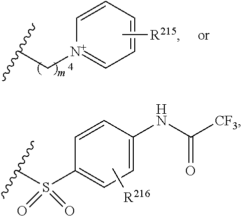
wherein m⁴ is 1, 2, 3, 4 or 5, R²⁰⁹, R²¹⁰, R²¹⁴, R²¹⁵ and R²¹⁶ are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and R²⁰⁹ and R²¹⁰, may alternately be and independently as a pair be a 3-7 membered substituted heterocarbocyclic ring or a 3-7 membered unsubstituted heterocarbocyclic ring, and R²¹¹, R²¹² and R²¹³ are each independently unsubstituted C(₁₋₁₁)alkyl, or substituted C(₁₋₁₁)alkyl; and
R¹⁰², R¹⁰³, R¹⁰⁶, R¹⁰⁹, R¹¹⁰, R¹¹³, R¹¹⁹, R¹²⁰, R¹²³, R¹²⁶, R¹²⁷, R¹³⁰, R¹³³, R¹³⁴, R¹³⁷, R¹⁴⁰, R¹⁴¹, R¹⁴⁴, R¹⁴⁷, R¹⁴⁸, R¹⁵¹, R¹⁵⁴, R¹⁵⁵, R¹⁵⁸, R¹⁶⁴, R¹⁶⁵, R¹⁶⁸, R¹⁷⁴, R¹⁷⁵, R¹⁷⁸, R¹⁸⁴, R¹⁸⁵, R¹⁸⁸, R¹⁹⁴, R¹⁹⁵, R¹⁹⁸, R²⁰⁴, R²⁰⁵ and R²⁰⁸ are each independently substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl;
(i) provided that G⁵ is absent only when G², G³ and G⁴ together form the ring moiety 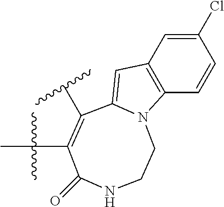 [SMILES: */C1=C(\*)c2cc3cc(Cl)ccc3n2CCNC1=O]
and G⁵ is absent when G², G³ and G⁴ together form the ring moiety 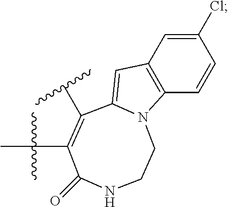 [SMILES: */C1=C(\*)c2cc3cc(Cl)ccc3n2CCNC1=O]
(ii) provided that when G³ is N, CH, or CG⁹ where G⁹ is C(O)OR⁹ and R⁹ is unsubstituted C₍₁₋₆₎ alkyl, G⁴ is other than 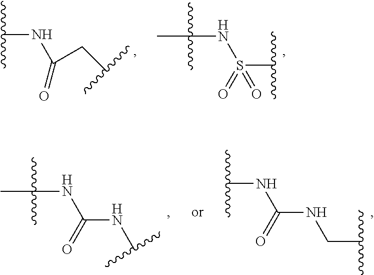 [SMILES: *CC(=O)N* ; *NS(*)(=O)=O ; *NC(=O)N* ; *CNC(=O)N*]
and G⁵ is 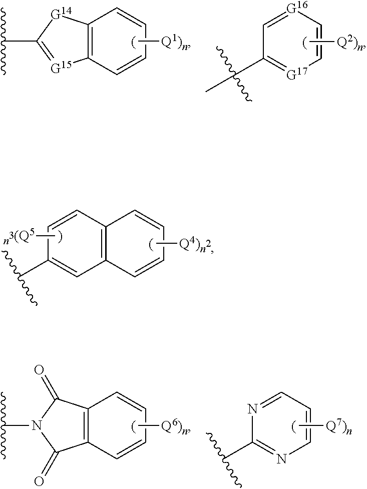
or a 5-membered heteroaryl optionally substituted with (Q⁸)ₙ and containing 1 or 2 heteroatoms each heteroatom independently selected from N, O and S, then n is at least 1 or n²+n³ is at least 1, and
(a) when n is 1 or n²+n³=1, then Q¹, Q², Q⁴, Q⁵, Q⁶, Q⁷ or Q⁸ is independently selected from the group consisting of —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NR^{104′}R^{105′}, and —NHC(O)R^{106′},
wherein R^{26′} is independently selected from the group consisting of substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₂₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl;
each R^{27′}, R^{28′}, R^{100′}, R^{104′} and R^{105′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; or each pair: a) R^{27′} and R^{28′}, or b) R^{104′} and R^{105′} may alternately be and independently as a pair be a 3-7 membered substituted heterocarbocyclic ring or a 3-7 membered unsubstituted heterocarbocyclic ring;
R^{101′} is H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, unsubstituted C(₂₋₁₁)heteroaralkyl, substituted C(₁₋₆)alkyl-NR^{209′}R^{210′}, unsubstituted C(₁₋₆)alkyl-NR^{209′}R^{210′}, substituted C(₁₋₆)alkyl-N⁺R^{211′}R^{212′}R^{213′}, unsubstituted C(₁₋₆)alkyl-N⁺R^{211′}R^{212′}R^{213′}, substituted C(₁₋₆)alkyl-OR^{214′}, unsubstituted C(₁₋₆)alkyl-OR^{214′}, 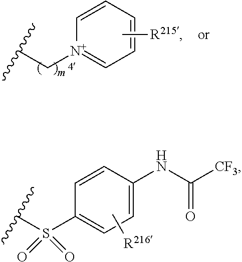
wherein m^{4′} is 1, 2, 3, 4 or 5, R^{209′}, R^{210′}, R^{214′}, R^{215′} and R^{216′} are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and R^{209′} and R^{210′}, may alternately be and independently as a pair be a 3-7 membered substituted heterocarbocyclic ring or a 3-7 membered unsubstituted heterocarbocyclic ring, and R^{211′}, R^{212′} and R^{213′} are each independently unsubstituted C(₁₋₁₁)alkyl, or substituted C(₁₋₁₁)alkyl; and
R^{102′}, R^{103′}, and R^{106′} are each independently substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and
(b) when n is at least 2 or n²+n³ is at least 2, then a first Q¹, Q², Q⁴, Q⁵, Q⁶, Q⁷ or Q⁸ is independently selected from the group consisting of —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NR^{104′}R^{105′}, and —NHC(O)R^{106′},
wherein each of R^{26′}, R^{27′}, R^{28′}, R^{100′}, R^{101′}, R^{102′}, R^{103′}, R^{104′}, R^{105′}, and R^{106′} is as defined above; and
the remaining Q¹, Q², Q³, Q⁴, Q⁵, Q⁷ or Q⁸ are each independently selected from the group consisting of halogen, —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, NR^{104′}R^{105′}, —NHC(O)R^{106′}, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
wherein each R^{26′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and
each of R^{27′}, R^{28′}, R^{100′}, R^{101′}, R^{102′}, R^{103′}, R^{104′}, R^{105′}, and R^{106′} is as defined above; and
(iii) provided that when G³ is N, CH, or CG⁹ where G⁹ is C(O)OR⁹ and R⁹ is unsubstituted C₍₁₋₆₎ alkyl, G⁴ is other than 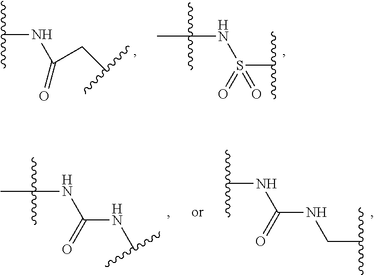 [SMILES: *CC(=O)N* ; *NS(*)(=O)=O ; *NC(=O)N* ; *CNC(=O)N*]
and G⁵ is 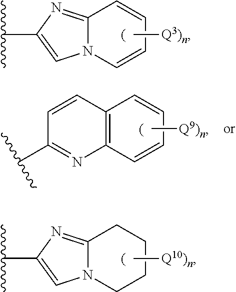
then n is at least 1
wherein each of Q³, Q⁹ and Q¹⁰ is as defined above, and
wherein the compound, or salt thereof, has anti-bacterial activity.

35. The method of claim 34 wherein G¹ is NH or S.

36. The method of claim 34 wherein G⁴ is selected from the group consisting of: a bond,

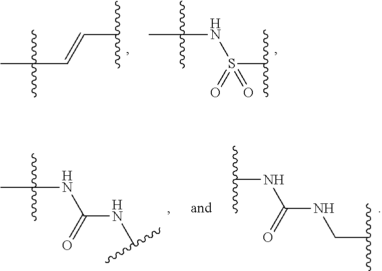

37. The method of claim 34 wherein G⁴ is selected from the group consisting of

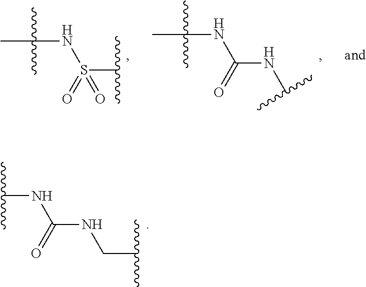 [SMILES: *NS(*)(=O)=O ; *NC(=O)N* ; *CNC(=O)N*]

38. The method of claim 34 wherein G⁴ is selected from the group consisting of: a bond, and

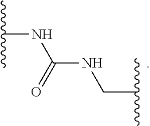 [SMILES: *CNC(=O)N*]

39. The method of claim 34 wherein G¹ is S and G⁴ is

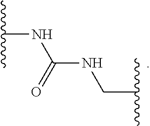 [SMILES: *CNC(=O)N*]

40. The method of claim 34 wherein G¹ is NH, and G⁴ is a bond.

41. The method of claim 40 wherein G³ is CG⁹ or CH.

42. The method of claim 41 wherein G⁵ is

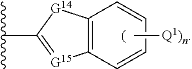

43. The method of claim 42 wherein G¹⁴ is NH and G¹⁵ is CH.

44. The method of claim 43 wherein n is at least 2.

45. The method of claim 44 wherein at least one Q¹ is selected from the group consisting of: —OR²⁶, —O—(C₁₋₆)alkyl-NR²⁷R²⁸, —O—(C₁₋₆)alkyl-C(O)OR¹⁰⁰, —O—(C₁₋₆)alkyl-C(O)NHR¹⁰¹, —O—(C₁₋₆)alkyl-OC(O)R¹⁰², and —O—(C₁₋₆)alkyl-OS(O)₂R¹⁰³.

46. The method of claim 45 wherein at least one Q¹ is halogen.

47. The method of claim 46 wherein at least one Q¹ is —O—(C₁₋₆)alkyl-C(O)NHR¹⁰¹.

48. The method of claim 47 wherein R¹⁰¹ is selected from the group consisting of: unsubstituted C(₁₋₆)alkyl-NR²⁰⁹R²¹⁰, unsubstituted C(₁₋₆)alkyl-N⁺R²¹¹R²¹²R²¹³, unsubstituted C(₁₋₆)alkyl-OR²¹⁴,

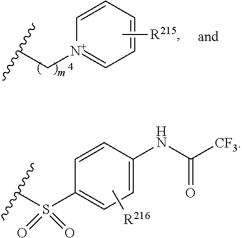

49. The method of claim 48 wherein at least one Q¹ is Cl.

50. The method of any one of claims 44 to 49 wherein n is 2.

51. The method of claim 43 wherein n is at least 1.

52. The method of claim 51 wherein at least 1 Q¹ is a halogen.

53. The method of claim 52 wherein G⁹ is —C(NOH)C(R²¹)(R²²)(R²³) or C(NOH)N(R²⁴)(R²⁵).

54. The method of claim 53 wherein R²¹, R²² and R²³ are each F.

55. The method of claim 53 wherein R²⁴ and R²⁵ are H.

56. The method of claim 39 wherein G⁵ is selected from the group consisting of:

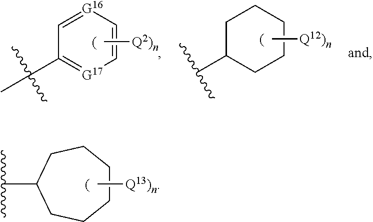

57. The method of claim 56 wherein G⁵ is

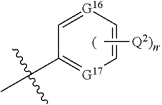

58. The method of claim 57 wherein G¹⁶ is CH and G¹⁷ is CH.

59. The method of claim 58 wherein n is 0, 1 or 2.

60. The method of claim 59 wherein n is at least one 1 and Q² is is selected from the group consisting of: halogen, NR¹¹¹R¹¹², NHC(O)R¹¹³, and substituted C₍₁₋₆₎ alkyl.

61. The method of claim 60 wherein the substituted C₍₁₋₆₎ alkyl is a halogen substituted methyl group.

62. The method of claim 61 wherein the halogen substituted methyl group is CF₃.

63. The method of any one of claims 59 to 62 wherein n is 1.

64. The method of any one of claims 59 to 62 wherein n is 2.

65. The method of any one of claims 60 to 64 wherein at least one Q² is halogen.

66. A method of treating a subject known to have or suspected of having a bacterial infection, the method comprising administering to the subject an effective amount of a compound selected from the group consisting of:
TABLE 2

| Compound | |
|---|---|
| # | Chemical Structure |
| 1 | 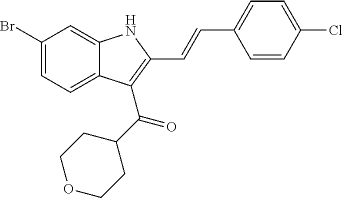 [SMILES: O=C(c1c(/C=C/c2ccc(Cl)cc2)[nH]c2cc(Br)ccc12)C1CCOCC1] |
| 2 | 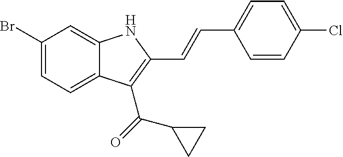 [SMILES: O=C(c1c(/C=C/c2ccc(Cl)cc2)[nH]c2cc(Br)ccc12)C1CC1] |
| 3 | 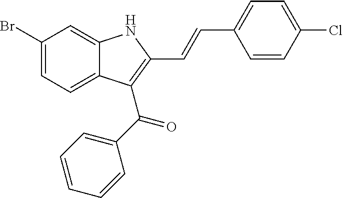 [SMILES: O=C(c1ccccc1)c1c(/C=C/c2ccc(Cl)cc2)[nH]c2cc(Br)ccc12] |
| 4 | 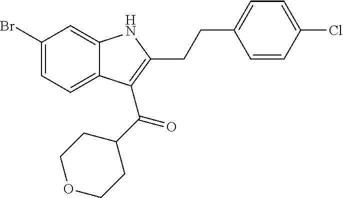 [SMILES: O=C(c1c(CCc2ccc(Cl)cc2)[nH]c2cc(Br)ccc12)C1CCOCC1] |
| 5 | 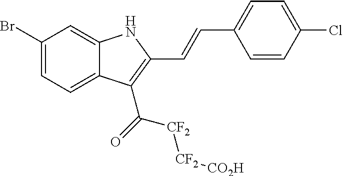 [SMILES: O=C(O)C(F)(F)C(F)(F)C(=O)c1c(/C=C/c2ccc(Cl)cc2)[nH]c2cc(Br)ccc12] |
| 6 | 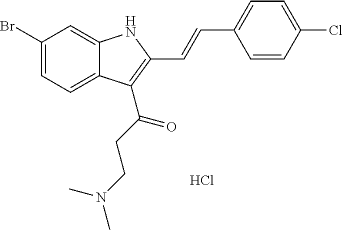 [SMILES: CN(C)CCC(=O)c1c(/C=C/c2ccc(Cl)cc2)[nH]c2cc(Br)ccc12 ; Cl] |
| 7 | 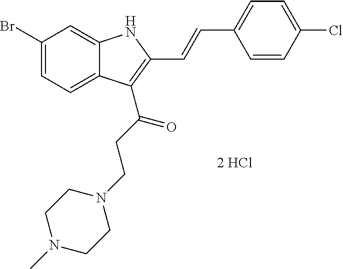 [SMILES: CN1CCN(CCC(=O)c2c(/C=C/c3ccc(Cl)cc3)[nH]c3cc(Br)ccc23)CC1] |
| 8 | 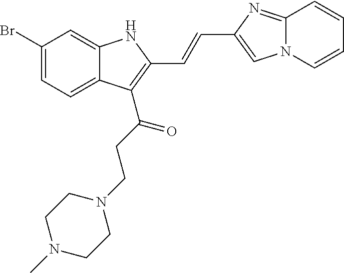 [SMILES: CN1CCN(CCC(=O)c2c(/C=C/c3cn4ccccc4n3)[nH]c3cc(Br)ccc23)CC1] |
| 9 | 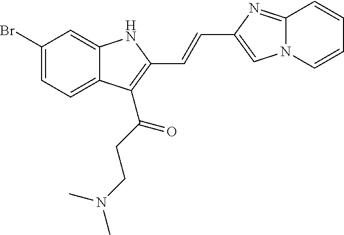 [SMILES: CN(C)CCC(=O)c1c(/C=C/c2cn3ccccc3n2)[nH]c2cc(Br)ccc12] |
| 10 | 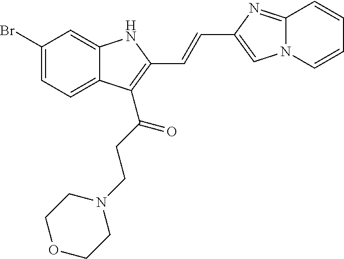 [SMILES: O=C(CCN1CCOCC1)c1c(/C=C/c2cn3ccccc3n2)[nH]c2cc(Br)ccc12] |
| 11 | 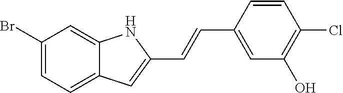 [SMILES: Oc1cc(/C=C/c2cc3ccc(Br)cc3[nH]2)ccc1Cl] |
| 12 | 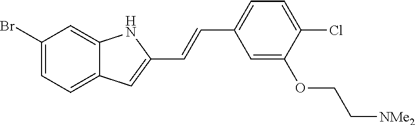 [SMILES: CN(C)CCOc1cc(/C=C/c2cc3ccc(Br)cc3[nH]2)ccc1Cl] |
| 13 | 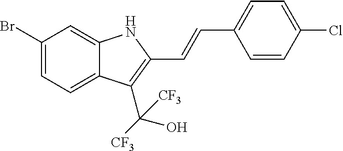 [SMILES: OC(c1c(/C=C/c2ccc(Cl)cc2)[nH]c2cc(Br)ccc12)(C(F)(F)F)C(F)(F)F] |
| 14 | 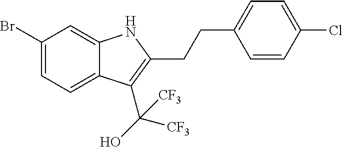 [SMILES: OC(c1c(CCc2ccc(Cl)cc2)[nH]c2cc(Br)ccc12)(C(F)(F)F)C(F)(F)F] |
| 15 | 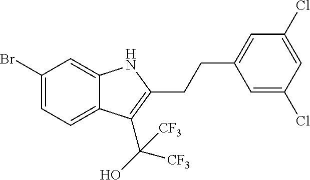 [SMILES: OC(c1c(CCc2cc(Cl)cc(Cl)c2)[nH]c2cc(Br)ccc12)(C(F)(F)F)C(F)(F)F] |
| 16 | 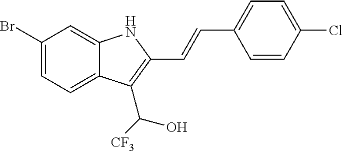 [SMILES: OC(c1c(/C=C/c2ccc(Cl)cc2)[nH]c2cc(Br)ccc12)C(F)(F)F] |
| 17 | 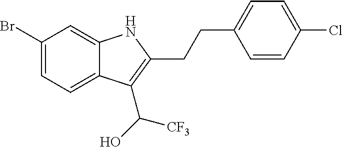 [SMILES: OC(c1c(CCc2ccc(Cl)cc2)[nH]c2cc(Br)ccc12)C(F)(F)F] |
| 18 | 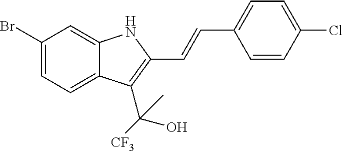 [SMILES: CC(O)(c1c(/C=C/c2ccc(Cl)cc2)[nH]c2cc(Br)ccc12)C(F)(F)F] |
| 19 | 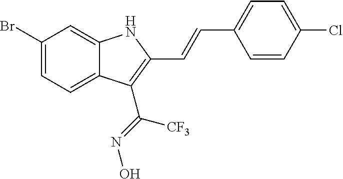 [SMILES: O/N=C(/c1c(/C=C/c2ccc(Cl)cc2)[nH]c2cc(Br)ccc12)C(F)(F)F] |
| 20 | 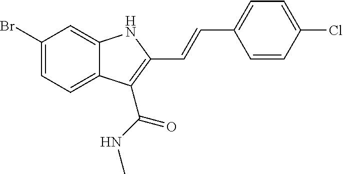 [SMILES: CNC(=O)c1c(/C=C/c2ccc(Cl)cc2)[nH]c2cc(Br)ccc12] |
| 21 | 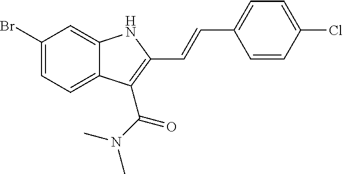 [SMILES: CN(C)C(=O)c1c(/C=C/c2ccc(Cl)cc2)[nH]c2cc(Br)ccc12] |
| 22 | 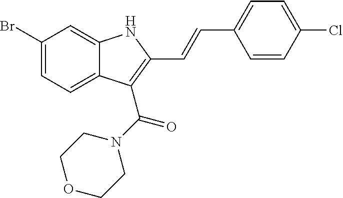 [SMILES: O=C(c1c(/C=C/c2ccc(Cl)cc2)[nH]c2cc(Br)ccc12)N1CCOCC1] |
| 23 | 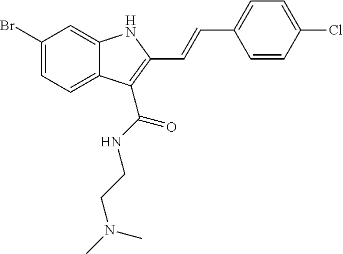 [SMILES: CN(C)CCNC(=O)c1c(/C=C/c2ccc(Cl)cc2)[nH]c2cc(Br)ccc12] |
| 24 | 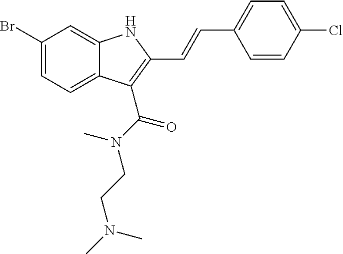 [SMILES: CN(C)CCN(C)C(=O)c1c(/C=C/c2ccc(Cl)cc2)[nH]c2cc(Br)ccc12] |
| 25 | 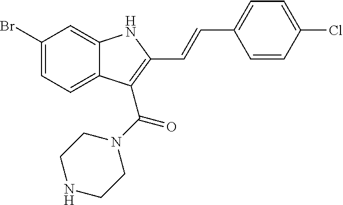 [SMILES: O=C(c1c(/C=C/c2ccc(Cl)cc2)[nH]c2cc(Br)ccc12)N1CCNCC1] |
| 26 | 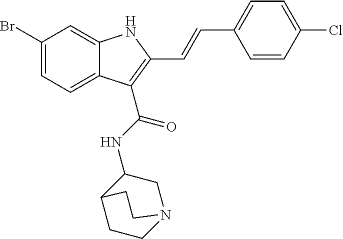 [SMILES: O=C(NC1CN2CCC1CC2)c1c(/C=C/c2ccc(Cl)cc2)[nH]c2cc(Br)ccc12] |
| 27 | 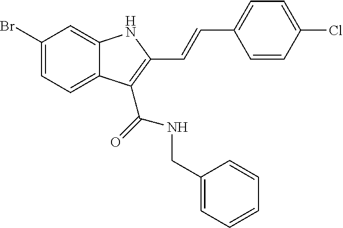 [SMILES: O=C(NCc1ccccc1)c1c(/C=C/c2ccc(Cl)cc2)[nH]c2cc(Br)ccc12] |
| 28 | 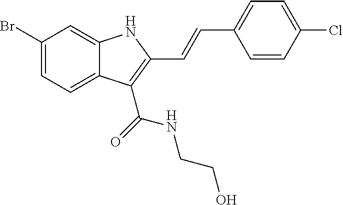 [SMILES: O=C(NCCO)c1c(/C=C/c2ccc(Cl)cc2)[nH]c2cc(Br)ccc12] |
| 29 | 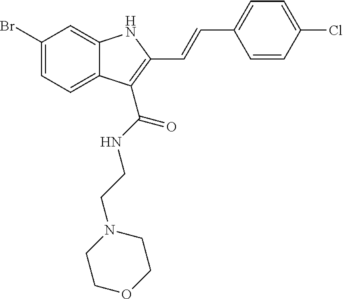 [SMILES: O=C(NCCN1CCOCC1)c1c(/C=C/c2ccc(Cl)cc2)[nH]c2cc(Br)ccc12] |
| 30 | 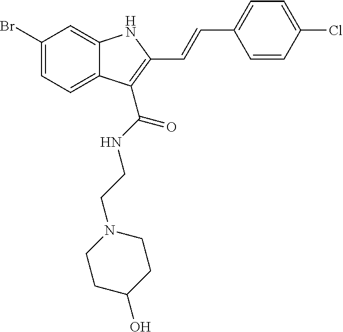 [SMILES: O=C(NCCN1CCC(O)CC1)c1c(/C=C/c2ccc(Cl)cc2)[nH]c2cc(Br)ccc12] |
| 31 | 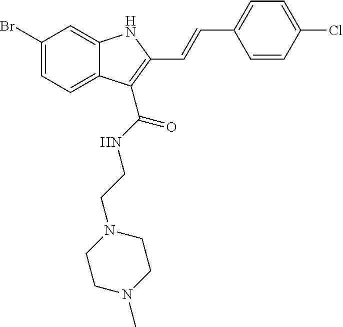 [SMILES: CN1CCN(CCNC(=O)c2c(/C=C/c3ccc(Cl)cc3)[nH]c3cc(Br)ccc23)CC1] |
| 32 | 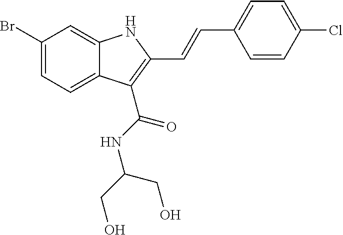 [SMILES: O=C(NC(CO)CO)c1c(/C=C/c2ccc(Cl)cc2)[nH]c2cc(Br)ccc12] |
| 33 | 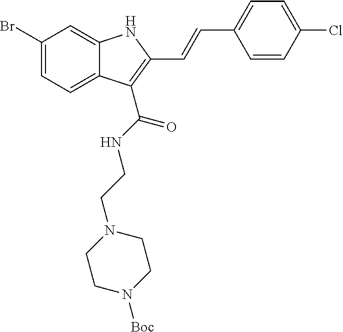 [SMILES: CC(C)(C)OC(=O)N1CCN(CCNC(=O)c2c(/C=C/c3ccc(Cl)cc3)[nH]c3cc(Br)ccc23)CC1] |
| 34 | 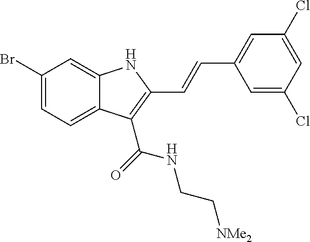 [SMILES: CN(C)CCNC(=O)c1c(/C=C/c2cc(Cl)cc(Cl)c2)[nH]c2cc(Br)ccc12] |
| 35 | 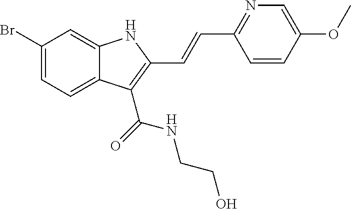 [SMILES: COc1ccc(/C=C/c2[nH]c3cc(Br)ccc3c2C(=O)NCCO)nc1] |
| 36 | 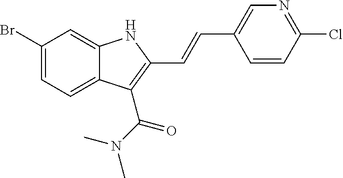 [SMILES: CN(C)C(=O)c1c(/C=C/c2ccc(Cl)nc2)[nH]c2cc(Br)ccc12] |
| 37 | 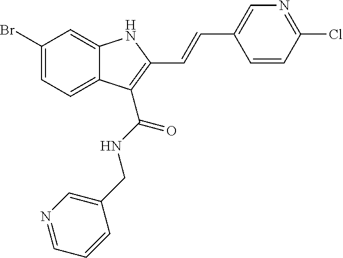 [SMILES: O=C(NCc1cccnc1)c1c(/C=C/c2ccc(Cl)nc2)[nH]c2cc(Br)ccc12] |
| 38 | 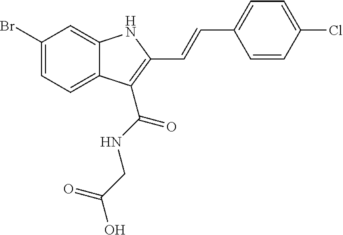 [SMILES: O=C(O)CNC(=O)c1c(/C=C/c2ccc(Cl)cc2)[nH]c2cc(Br)ccc12] |
| 39 | 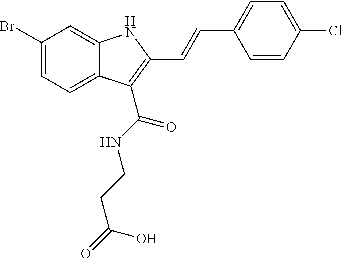 [SMILES: O=C(O)CCNC(=O)c1c(/C=C/c2ccc(Cl)cc2)[nH]c2cc(Br)ccc12] |
| 40 | 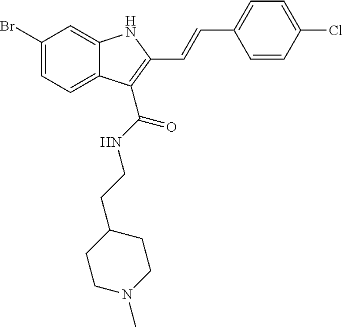 [SMILES: CN1CCC(CCNC(=O)c2c(/C=C/c3ccc(Cl)cc3)[nH]c3cc(Br)ccc23)CC1] |
| 41 | 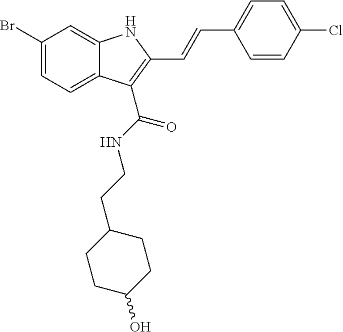 [SMILES: O=C(NCCC1CCC(O)CC1)c1c(/C=C/c2ccc(Cl)cc2)[nH]c2cc(Br)ccc12] |
| 42 | 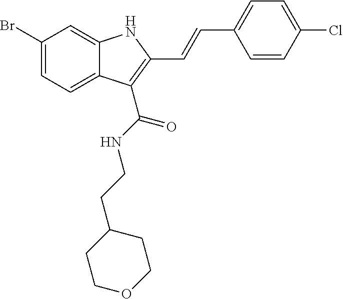 [SMILES: O=C(NCCC1CCOCC1)c1c(/C=C/c2ccc(Cl)cc2)[nH]c2cc(Br)ccc12] |
| 43 | 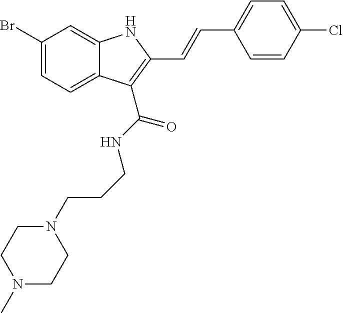 [SMILES: CN1CCN(CCCNC(=O)c2c(/C=C/c3ccc(Cl)cc3)[nH]c3cc(Br)ccc23)CC1] |
| 44 | 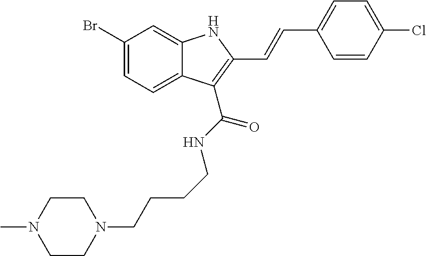 [SMILES: CN1CCN(CCCCNC(=O)c2c(/C=C/c3ccc(Cl)cc3)[nH]c3cc(Br)ccc23)CC1] |
| 45 | 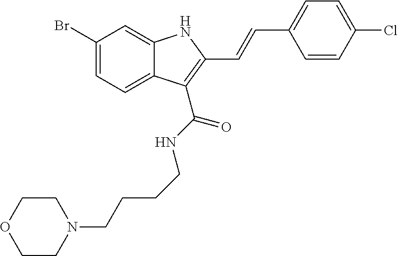 [SMILES: O=C(NCCCCN1CCOCC1)c1c(/C=C/c2ccc(Cl)cc2)[nH]c2cc(Br)ccc12] |
| 46 | 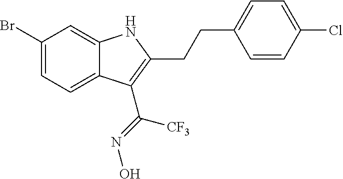 [SMILES: O/N=C(/c1c(CCc2ccc(Cl)cc2)[nH]c2cc(Br)ccc12)C(F)(F)F] |
| 47 | 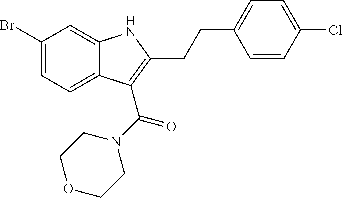 [SMILES: O=C(c1c(CCc2ccc(Cl)cc2)[nH]c2cc(Br)ccc12)N1CCOCC1] |
| 48 | 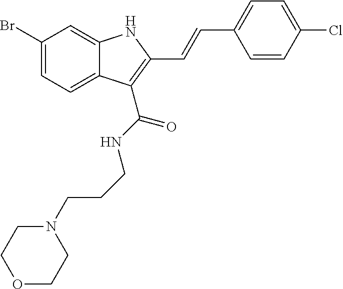 [SMILES: O=C(NCCCN1CCOCC1)c1c(/C=C/c2ccc(Cl)cc2)[nH]c2cc(Br)ccc12] |
| 49 | 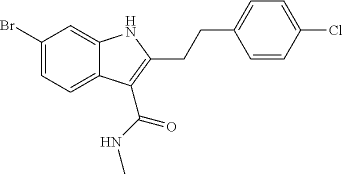 [SMILES: CNC(=O)c1c(CCc2ccc(Cl)cc2)[nH]c2cc(Br)ccc12] |
| 50 | 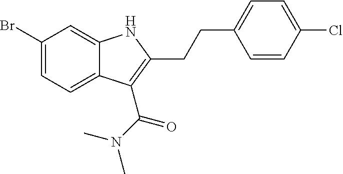 [SMILES: CN(C)C(=O)c1c(CCc2ccc(Cl)cc2)[nH]c2cc(Br)ccc12] |
| 51 | 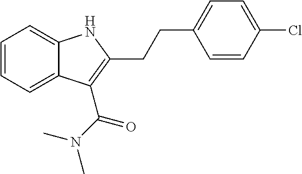 [SMILES: CN(C)C(=O)c1c(CCc2ccc(Cl)cc2)[nH]c2ccccc12] |
| 52 | 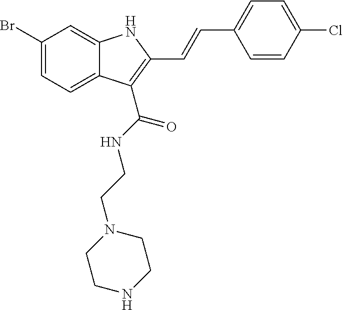 [SMILES: O=C(NCCN1CCNCC1)c1c(/C=C/c2ccc(Cl)cc2)[nH]c2cc(Br)ccc12] |
| 53 | 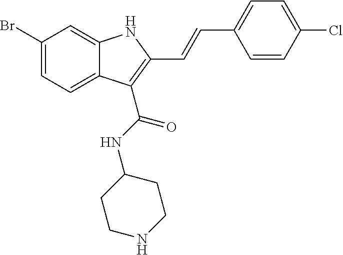 [SMILES: O=C(NC1CCNCC1)c1c(/C=C/c2ccc(Cl)cc2)[nH]c2cc(Br)ccc12] |
| 54 | 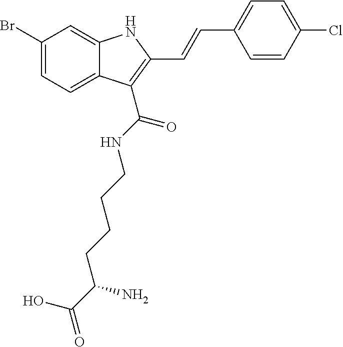 [SMILES: N[C@@H](CCCCNC(=O)c1c(/C=C/c2ccc(Cl)cc2)[nH]c2cc(Br)ccc12)C(=O)O] |
| 55 | 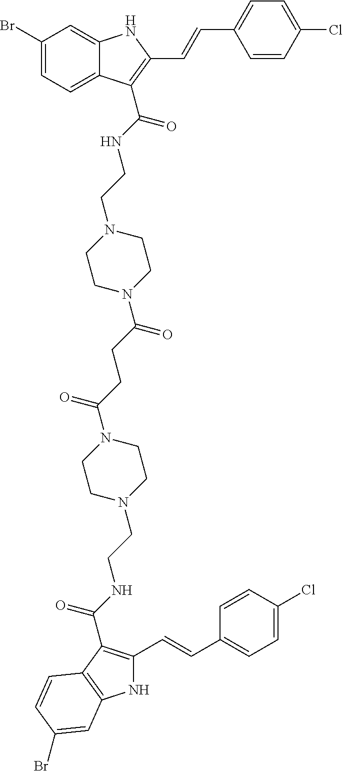 [SMILES: O=C(NCCN1CCN(C(=O)CCC(=O)N2CCN(CCNC(=O)c3c(/C=C/c4ccc(Cl)cc4)[nH]c4cc(Br)ccc34)CC2)CC1)c1c(/C=C/c2ccc(Cl)cc2)[nH]c2cc(Br)ccc12] |
| 56 | 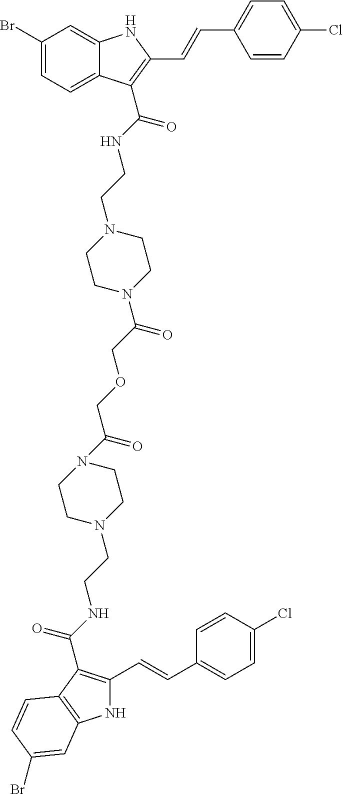 [SMILES: O=C(NCCN1CCN(C(=O)COCC(=O)N2CCN(CCNC(=O)c3c(/C=C/c4ccc(Cl)cc4)[nH]c4cc(Br)ccc34)CC2)CC1)c1c(/C=C/c2ccc(Cl)cc2)[nH]c2cc(Br)ccc12] |
| 57 | 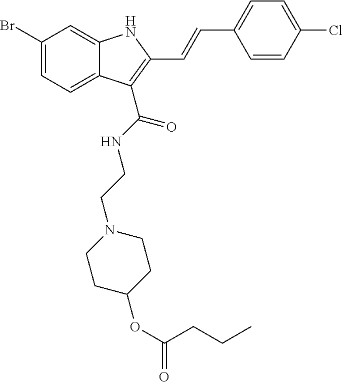 [SMILES: CCCC(=O)OC1CCN(CCNC(=O)c2c(/C=C/c3ccc(Cl)cc3)[nH]c3cc(Br)ccc23)CC1] |
| 58 | 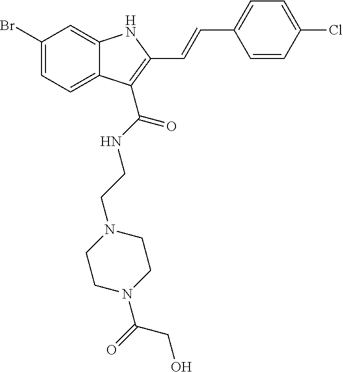 [SMILES: O=C(NCCN1CCN(C(=O)CO)CC1)c1c(/C=C/c2ccc(Cl)cc2)[nH]c2cc(Br)ccc12] |
| 59 | 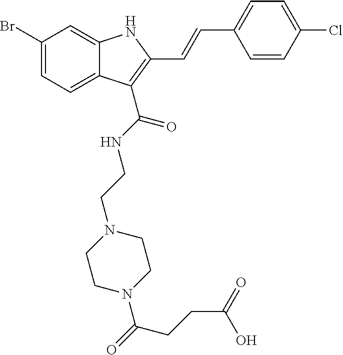 [SMILES: O=C(O)CCC(=O)N1CCN(CCNC(=O)c2c(/C=C/c3ccc(Cl)cc3)[nH]c3cc(Br)ccc23)CC1] |
| 60 | 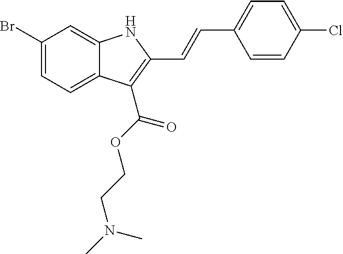 [SMILES: CN(C)CCOC(=O)c1c(/C=C/c2ccc(Cl)cc2)[nH]c2cc(Br)ccc12] |
| 61 | 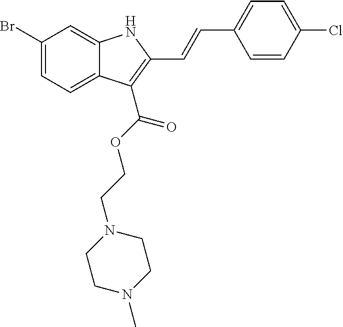 [SMILES: CN1CCN(CCOC(=O)c2c(/C=C/c3ccc(Cl)cc3)[nH]c3cc(Br)ccc23)CC1] |
| 62 | 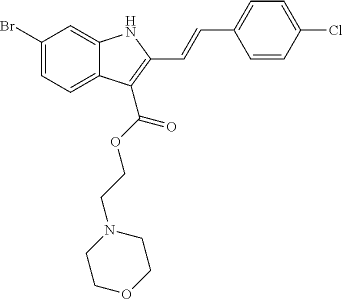 [SMILES: O=C(OCCN1CCOCC1)c1c(/C=C/c2ccc(Cl)cc2)[nH]c2cc(Br)ccc12] |
| 63 | 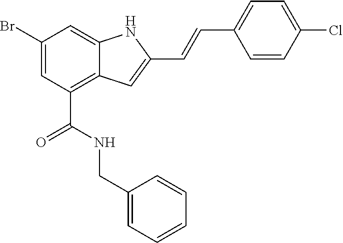 [SMILES: O=C(NCc1ccccc1)c1cc(Br)cc2[nH]c(/C=C/c3ccc(Cl)cc3)cc12] |
| 64 | 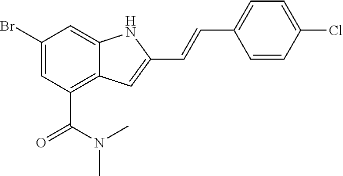 [SMILES: CN(C)C(=O)c1cc(Br)cc2[nH]c(/C=C/c3ccc(Cl)cc3)cc12] |
| 65 | 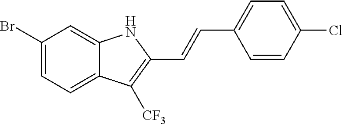 [SMILES: FC(F)(F)c1c(/C=C/c2ccc(Cl)cc2)[nH]c2cc(Br)ccc12] |
| 66 | 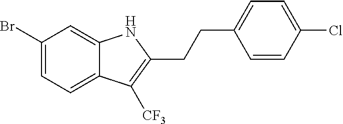 [SMILES: FC(F)(F)c1c(CCc2ccc(Cl)cc2)[nH]c2cc(Br)ccc12] |
| 67 | 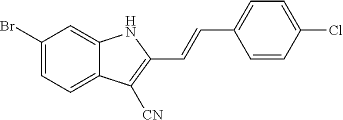 [SMILES: N#Cc1c(/C=C/c2ccc(Cl)cc2)[nH]c2cc(Br)ccc12] |
| 68 | 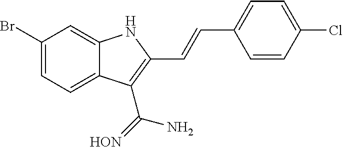 |
| 69 | 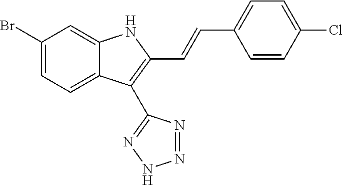 [SMILES: Clc1ccc(/C=C/c2[nH]c3cc(Br)ccc3c2-c2nn[nH]n2)cc1] |
| 70 | 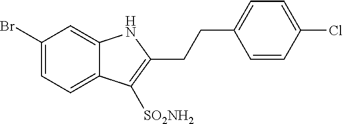 [SMILES: NS(=O)(=O)c1c(CCc2ccc(Cl)cc2)[nH]c2cc(Br)ccc12] |
| 71 | 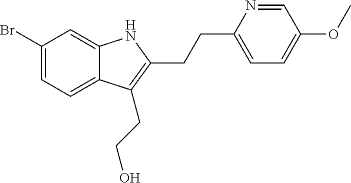 [SMILES: COc1ccc(CCc2[nH]c3cc(Br)ccc3c2CCO)nc1] |
| 72 | 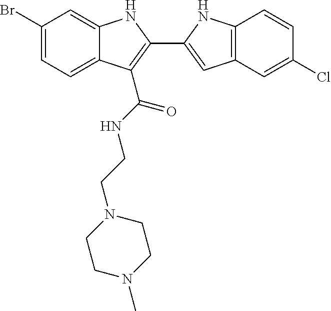 [SMILES: CN1CCN(CCNC(=O)c2c(-c3cc4cc(Cl)ccc4[nH]3)[nH]c3cc(Br)ccc23)CC1] |
| 73 | 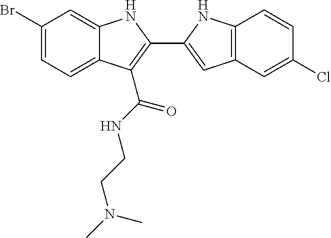 [SMILES: CN(C)CCNC(=O)c1c(-c2cc3cc(Cl)ccc3[nH]2)[nH]c2cc(Br)ccc12] |
| 74 | 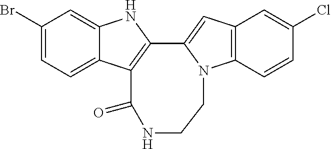 [SMILES: O=C1NCCn2c(cc3cc(Cl)ccc32)-c2[nH]c3cc(Br)ccc3c21] |
| 75 | 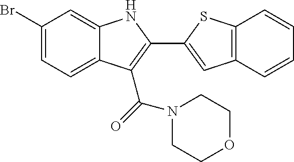 [SMILES: O=C(c1c(-c2cc3ccccc3s2)[nH]c2cc(Br)ccc12)N1CCOCC1] |
| 76 | 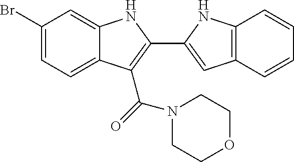 [SMILES: O=C(c1c(-c2cc3ccccc3[nH]2)[nH]c2cc(Br)ccc12)N1CCOCC1] |
| 77 | 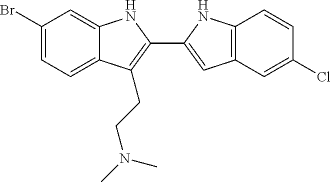 [SMILES: CN(C)CCc1c(-c2cc3cc(Cl)ccc3[nH]2)[nH]c2cc(Br)ccc12] |
| 78 | 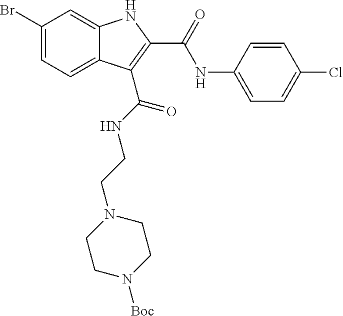 [SMILES: CC(C)(C)OC(=O)N1CCN(CCNC(=O)c2c(C(=O)Nc3ccc(Cl)cc3)[nH]c3cc(Br)ccc23)CC1] |
| 79 | 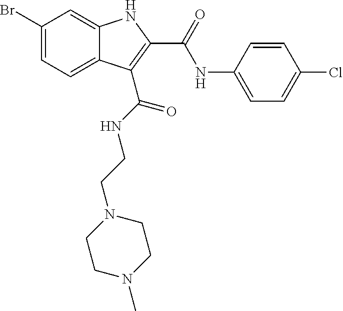 [SMILES: CN1CCN(CCNC(=O)c2c(C(=O)Nc3ccc(Cl)cc3)[nH]c3cc(Br)ccc23)CC1] |
| 80 | 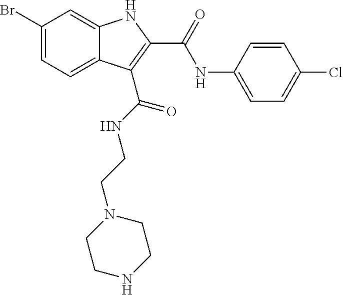 [SMILES: O=C(Nc1ccc(Cl)cc1)c1[nH]c2cc(Br)ccc2c1C(=O)NCCN1CCNCC1] |
| 81 | 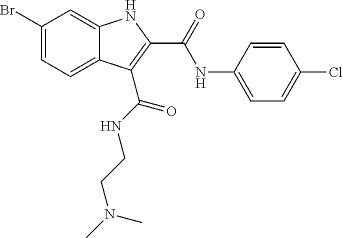 [SMILES: CN(C)CCNC(=O)c1c(C(=O)Nc2ccc(Cl)cc2)[nH]c2cc(Br)ccc12] |
| 82 | 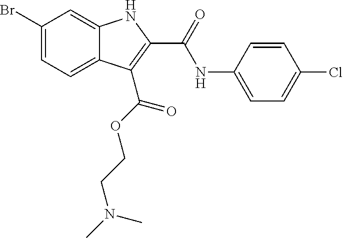 [SMILES: CN(C)CCOC(=O)c1c(C(=O)Nc2ccc(Cl)cc2)[nH]c2cc(Br)ccc12] |
| 83 | 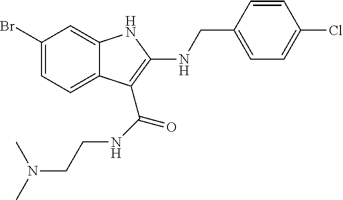 [SMILES: CN(C)CCNC(=O)c1c(NCc2ccc(Cl)cc2)[nH]c2cc(Br)ccc12] |
| 84 | 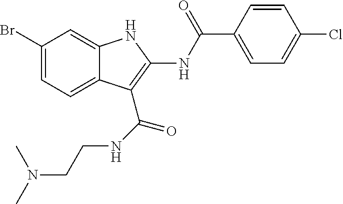 [SMILES: CN(C)CCNC(=O)c1c(NC(=O)c2ccc(Cl)cc2)[nH]c2cc(Br)ccc12] |
| 85 | 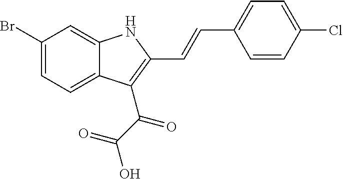 [SMILES: O=C(O)C(=O)c1c(/C=C/c2ccc(Cl)cc2)[nH]c2cc(Br)ccc12] |
| 86 | 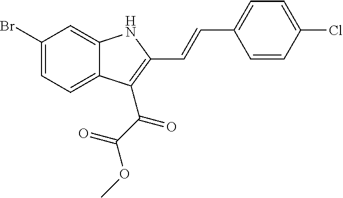 [SMILES: COC(=O)C(=O)c1c(/C=C/c2ccc(Cl)cc2)[nH]c2cc(Br)ccc12] |
| 87 | 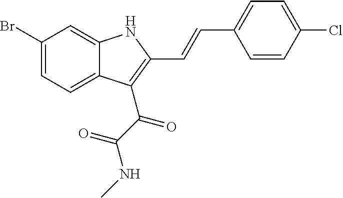 [SMILES: CNC(=O)C(=O)c1c(/C=C/c2ccc(Cl)cc2)[nH]c2cc(Br)ccc12] |
| 88 | 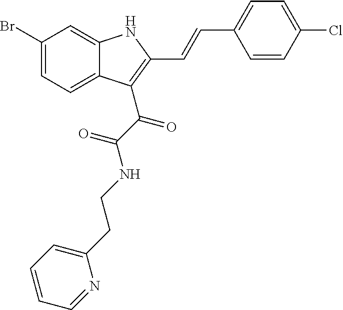 [SMILES: O=C(NCCc1ccccn1)C(=O)c1c(/C=C/c2ccc(Cl)cc2)[nH]c2cc(Br)ccc12] |
| 89 | 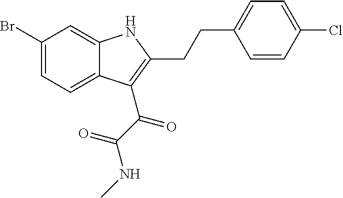 [SMILES: CNC(=O)C(=O)c1c(CCc2ccc(Cl)cc2)[nH]c2cc(Br)ccc12] |
| 90 | 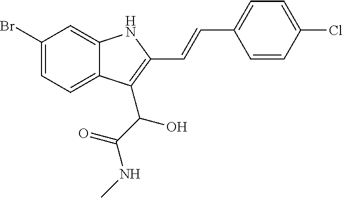 [SMILES: CNC(=O)C(O)c1c(/C=C/c2ccc(Cl)cc2)[nH]c2cc(Br)ccc12] |
| 91 | 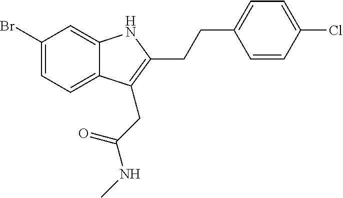 [SMILES: CNC(=O)Cc1c(CCc2ccc(Cl)cc2)[nH]c2cc(Br)ccc12] |
| 92 | 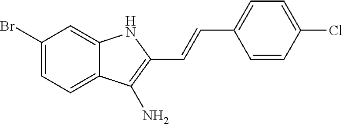 [SMILES: Nc1c(/C=C/c2ccc(Cl)cc2)[nH]c2cc(Br)ccc12] |
| 93 | 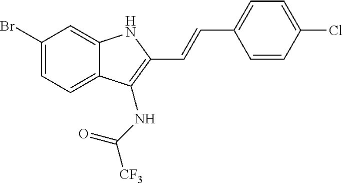 [SMILES: O=C(Nc1c(/C=C/c2ccc(Cl)cc2)[nH]c2cc(Br)ccc12)C(F)(F)F] |
| 94 | 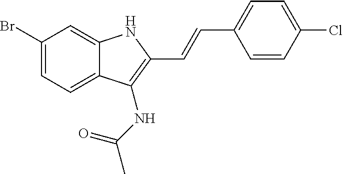 [SMILES: CC(=O)Nc1c(/C=C/c2ccc(Cl)cc2)[nH]c2cc(Br)ccc12] |
| 95 | 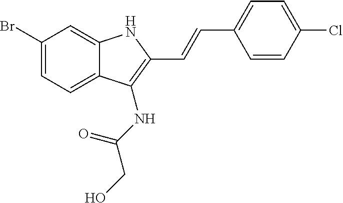 [SMILES: O=C(CO)Nc1c(/C=C/c2ccc(Cl)cc2)[nH]c2cc(Br)ccc12] |
| 96 | 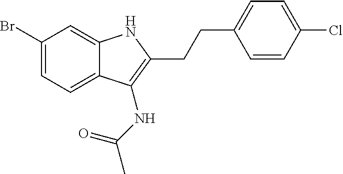 [SMILES: CC(=O)Nc1c(CCc2ccc(Cl)cc2)[nH]c2cc(Br)ccc12] |
| 97 | 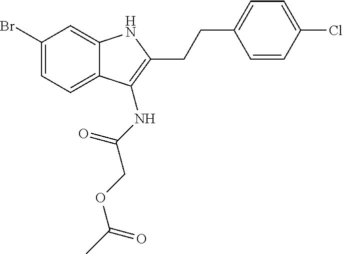 [SMILES: CC(=O)OCC(=O)Nc1c(CCc2ccc(Cl)cc2)[nH]c2cc(Br)ccc12] |
| 98 | 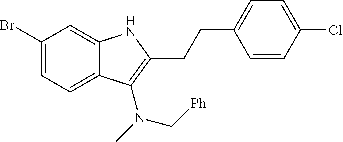 [SMILES: CN(Cc1ccccc1)c1c(CCc2ccc(Cl)cc2)[nH]c2cc(Br)ccc12] |
| 99 | 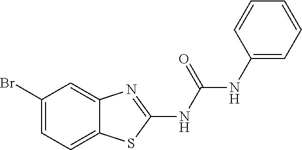 [SMILES: O=C(Nc1ccccc1)Nc1nc2cc(Br)ccc2s1] |
| 100 | 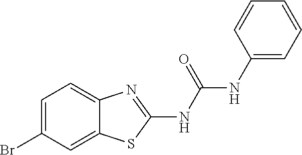 [SMILES: O=C(Nc1ccccc1)Nc1nc2ccc(Br)cc2s1] |
| 101 | 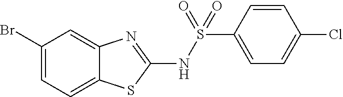 [SMILES: O=S(=O)(Nc1nc2cc(Br)ccc2s1)c1ccc(Cl)cc1] |
| 102 | 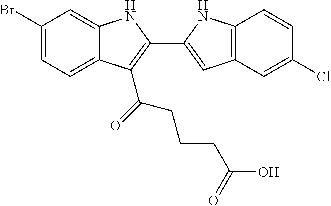 [SMILES: O=C(O)CCCC(=O)c1c(-c2cc3cc(Cl)ccc3[nH]2)[nH]c2cc(Br)ccc12] |
| 103 | 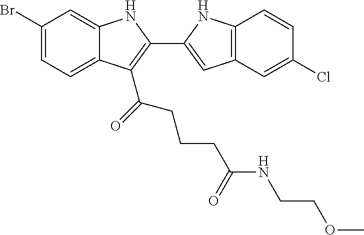 [SMILES: COCCNC(=O)CCCC(=O)c1c(-c2cc3cc(Cl)ccc3[nH]2)[nH]c2cc(Br)ccc12] |
| 104 | 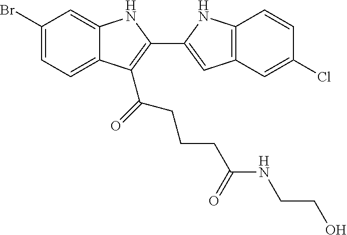 [SMILES: O=C(CCCC(=O)c1c(-c2cc3cc(Cl)ccc3[nH]2)[nH]c2cc(Br)ccc12)NCCO] |
| 105 | 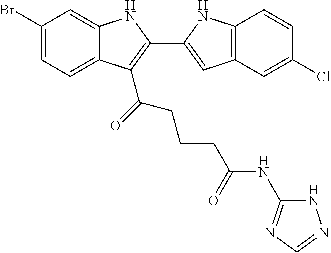 [SMILES: O=C(CCCC(=O)c1c(-c2cc3cc(Cl)ccc3[nH]2)[nH]c2cc(Br)ccc12)Nc1ncn[nH]1] |
| 106 | 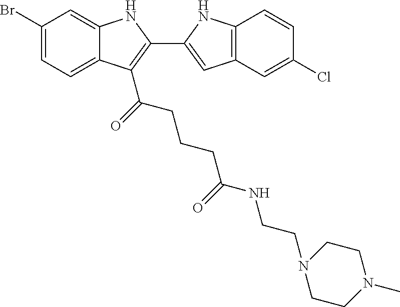 [SMILES: CN1CCN(CCNC(=O)CCCC(=O)c2c(-c3cc4cc(Cl)ccc4[nH]3)[nH]c3cc(Br)ccc23)CC1] |
| 107 | 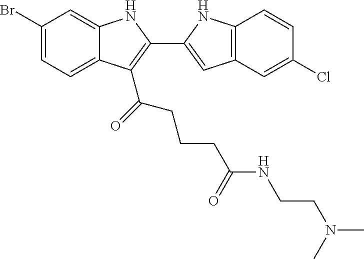 [SMILES: CN(C)CCNC(=O)CCCC(=O)c1c(-c2cc3cc(Cl)ccc3[nH]2)[nH]c2cc(Br)ccc12] |
| 108 | 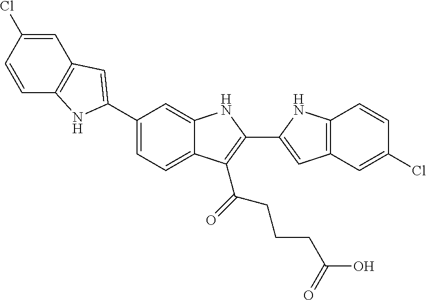 [SMILES: O=C(O)CCCC(=O)c1c(-c2cc3cc(Cl)ccc3[nH]2)[nH]c2cc(-c3cc4cc(Cl)ccc4[nH]3)ccc12] |
| 109 | 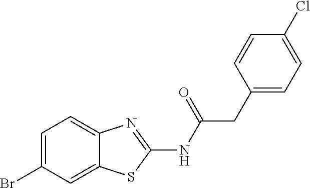 [SMILES: O=C(Cc1ccc(Cl)cc1)Nc1nc2ccc(Br)cc2s1] |
| 110 | 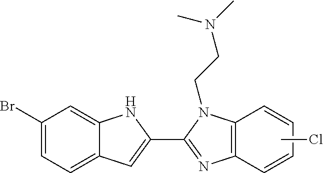 |
| 111 | 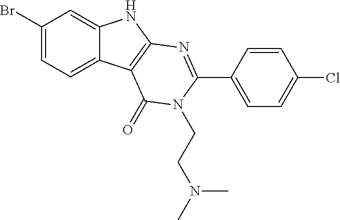 [SMILES: CN(C)CCn1c(-c2ccc(Cl)cc2)nc2[nH]c3cc(Br)ccc3c2c1=O] |
| 112 | 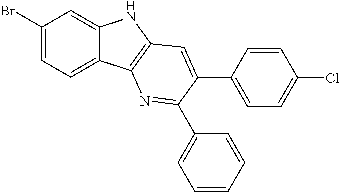 [SMILES: Clc1ccc(-c2cc3[nH]c4cc(Br)ccc4c3nc2-c2ccccc2)cc1] |
| 113 | 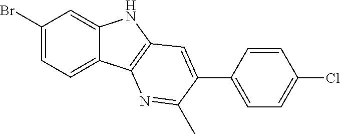 [SMILES: Cc1nc2c(cc1-c1ccc(Cl)cc1)[nH]c1cc(Br)ccc12] |
| 114 | 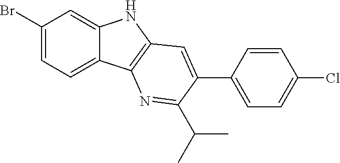 [SMILES: CC(C)c1nc2c(cc1-c1ccc(Cl)cc1)[nH]c1cc(Br)ccc12] |
| 115 | 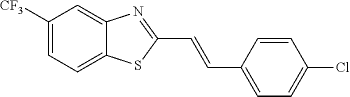 [SMILES: FC(F)(F)c1ccc2sc(/C=C/c3ccc(Cl)cc3)nc2c1] |
| 116 | 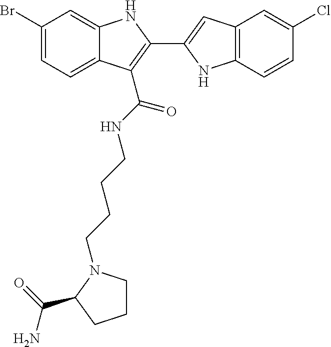 [SMILES: NC(=O)[C@@H]1CCCN1CCCCNC(=O)c1c(-c2cc3cc(Cl)ccc3[nH]2)[nH]c2cc(Br)ccc12] |
| 117 | 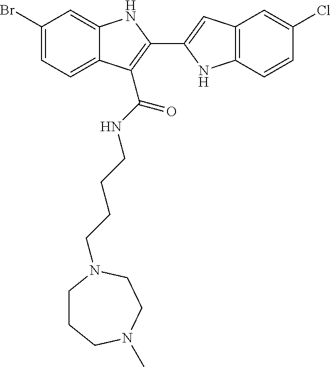 [SMILES: CN1CCCN(CCCCNC(=O)c2c(-c3cc4cc(Cl)ccc4[nH]3)[nH]c3cc(Br)ccc23)CC1] |
| 118 | 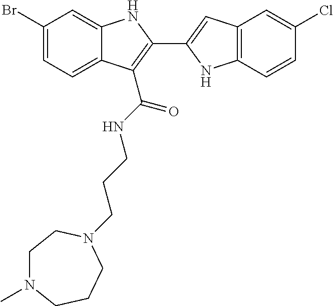 [SMILES: CN1CCCN(CCCNC(=O)c2c(-c3cc4cc(Cl)ccc4[nH]3)[nH]c3cc(Br)ccc23)CC1] |
| 119 | 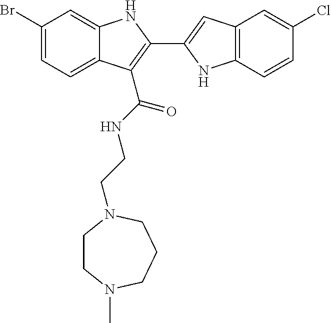 [SMILES: CN1CCCN(CCNC(=O)c2c(-c3cc4cc(Cl)ccc4[nH]3)[nH]c3cc(Br)ccc23)CC1] |
| 120 | 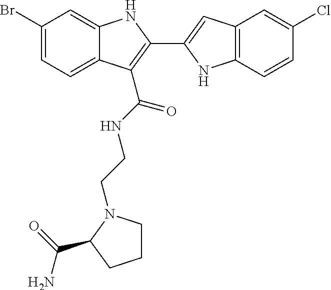 [SMILES: NC(=O)[C@@H]1CCCN1CCNC(=O)c1c(-c2cc3cc(Cl)ccc3[nH]2)[nH]c2cc(Br)ccc12] |
| 121 | 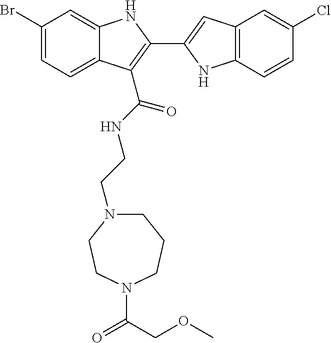 [SMILES: COCC(=O)N1CCCN(CCNC(=O)c2c(-c3cc4cc(Cl)ccc4[nH]3)[nH]c3cc(Br)ccc23)CC1] |
| 122 | 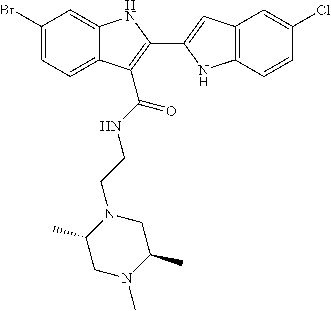 [SMILES: C[C@@H]1CN(CCNC(=O)c2c(-c3cc4cc(Cl)ccc4[nH]3)[nH]c3cc(Br)ccc23)[C@@H](C)CN1C] |
| 123 | 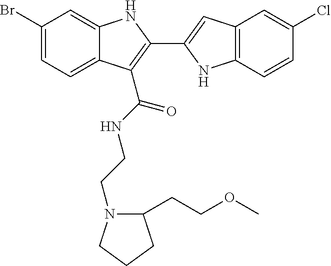 [SMILES: COCCC1CCCN1CCNC(=O)c1c(-c2cc3cc(Cl)ccc3[nH]2)[nH]c2cc(Br)ccc12] |
| 124 | 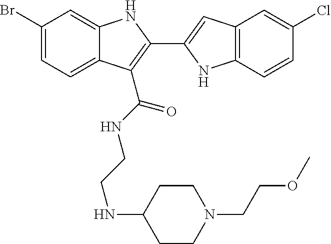 [SMILES: COCCN1CCC(NCCNC(=O)c2c(-c3cc4cc(Cl)ccc4[nH]3)[nH]c3cc(Br)ccc23)CC1] |
| 125 | 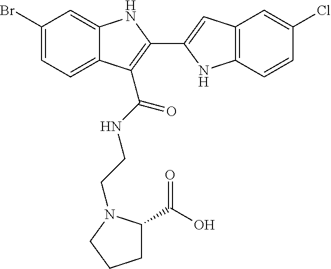 [SMILES: O=C(NCCN1CCC[C@H]1C(=O)O)c1c(-c2cc3cc(Cl)ccc3[nH]2)[nH]c2cc(Br)ccc12] |
| 126 | 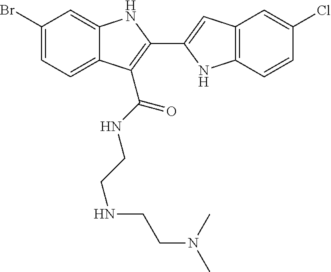 [SMILES: CN(C)CCNCCNC(=O)c1c(-c2cc3cc(Cl)ccc3[nH]2)[nH]c2cc(Br)ccc12] |
| 127 | 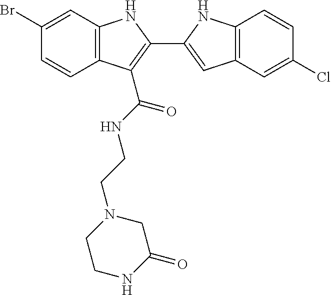 [SMILES: O=C1CN(CCNC(=O)c2c(-c3cc4cc(Cl)ccc4[nH]3)[nH]c3cc(Br)ccc23)CCN1] |
| 128 | 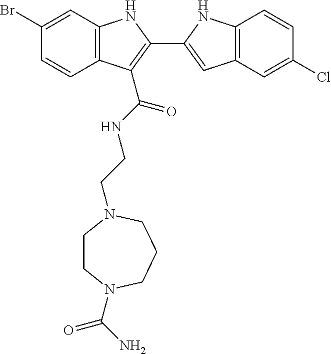 [SMILES: NC(=O)N1CCCN(CCNC(=O)c2c(-c3cc4cc(Cl)ccc4[nH]3)[nH]c3cc(Br)ccc23)CC1] |
| 129 | 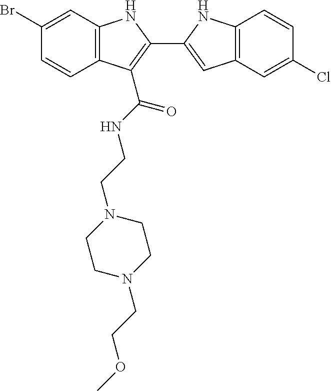 [SMILES: COCCN1CCN(CCNC(=O)c2c(-c3cc4cc(Cl)ccc4[nH]3)[nH]c3cc(Br)ccc23)CC1] |
| 130 | 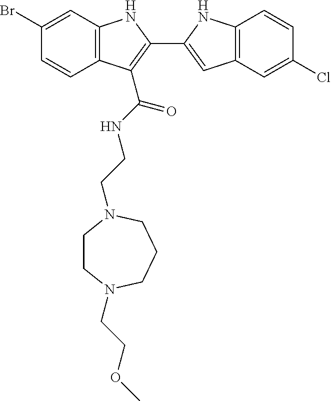 [SMILES: COCCN1CCCN(CCNC(=O)c2c(-c3cc4cc(Cl)ccc4[nH]3)[nH]c3cc(Br)ccc23)CC1] |
| 131 | 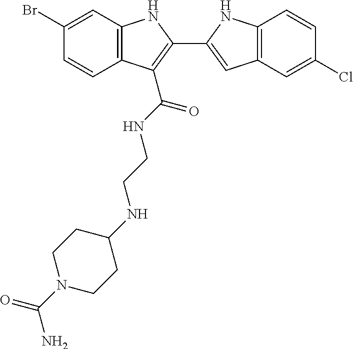 [SMILES: NC(=O)N1CCC(NCCNC(=O)c2c(-c3cc4cc(Cl)ccc4[nH]3)[nH]c3cc(Br)ccc23)CC1] |
| 132 | 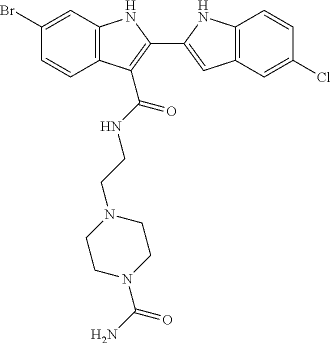 [SMILES: NC(=O)N1CCN(CCNC(=O)c2c(-c3cc4cc(Cl)ccc4[nH]3)[nH]c3cc(Br)ccc23)CC1] |
| 133 | 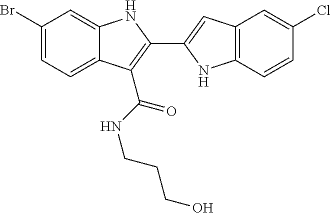 [SMILES: O=C(NCCCO)c1c(-c2cc3cc(Cl)ccc3[nH]2)[nH]c2cc(Br)ccc12] |
| 134 | 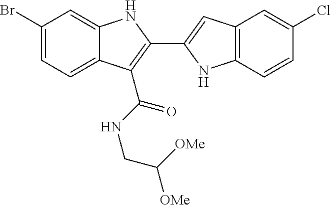 [SMILES: COC(CNC(=O)c1c(-c2cc3cc(Cl)ccc3[nH]2)[nH]c2cc(Br)ccc12)OC] |
| 135 | 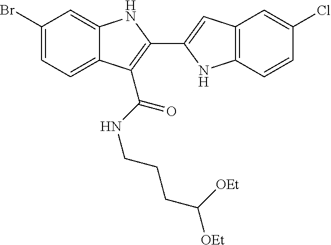 [SMILES: CCOC(CCCNC(=O)c1c(-c2cc3cc(Cl)ccc3[nH]2)[nH]c2cc(Br)ccc12)OCC] |
| 136 | 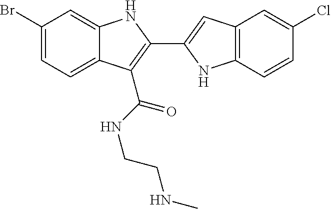 [SMILES: CNCCNC(=O)c1c(-c2cc3cc(Cl)ccc3[nH]2)[nH]c2cc(Br)ccc12] |
| 137 | 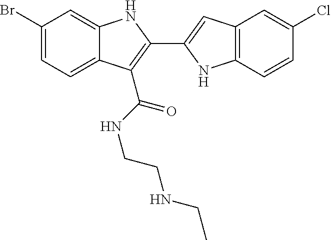 [SMILES: CCNCCNC(=O)c1c(-c2cc3cc(Cl)ccc3[nH]2)[nH]c2cc(Br)ccc12] |
| 138 | 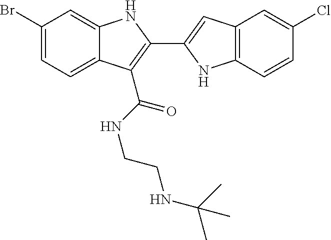 [SMILES: CC(C)(C)NCCNC(=O)c1c(-c2cc3cc(Cl)ccc3[nH]2)[nH]c2cc(Br)ccc12] |
| 139 | 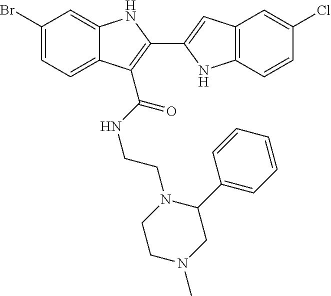 [SMILES: CN1CCN(CCNC(=O)c2c(-c3cc4cc(Cl)ccc4[nH]3)[nH]c3cc(Br)ccc23)C(c2ccccc2)C1] |
| 140 | 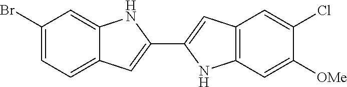 [SMILES: COc1cc2[nH]c(-c3cc4ccc(Br)cc4[nH]3)cc2cc1Cl] |
| 141 | 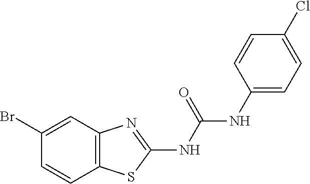 [SMILES: O=C(Nc1ccc(Cl)cc1)Nc1nc2cc(Br)ccc2s1] |
| 142 | 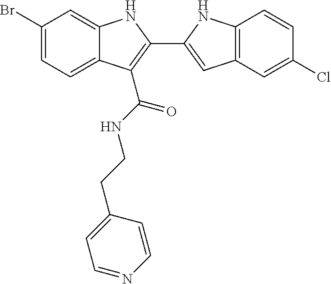 [SMILES: O=C(NCCc1ccncc1)c1c(-c2cc3cc(Cl)ccc3[nH]2)[nH]c2cc(Br)ccc12] |
| 143 | 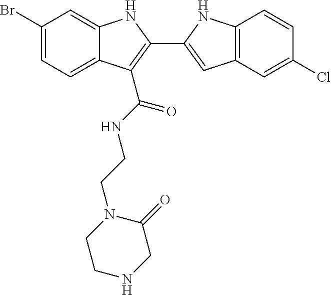 [SMILES: O=C(NCCN1CCNCC1=O)c1c(-c2cc3cc(Cl)ccc3[nH]2)[nH]c2cc(Br)ccc12] |
| 144 | 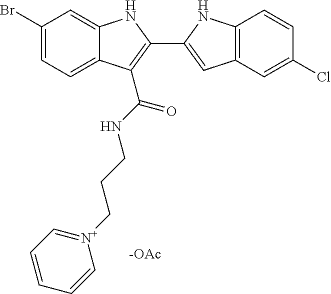 [SMILES: O=C(NCCC[n+]1ccccc1)c1c(-c2cc3cc(Cl)ccc3[nH]2)[nH]c2cc(Br)ccc12] |
| 145 | 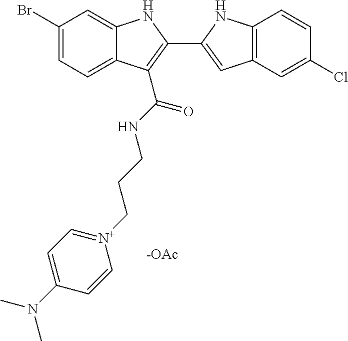 [SMILES: CN(C)c1cc[n+](CCCNC(=O)c2c(-c3cc4cc(Cl)ccc4[nH]3)[nH]c3cc(Br)ccc23)cc1] |
| 146 | 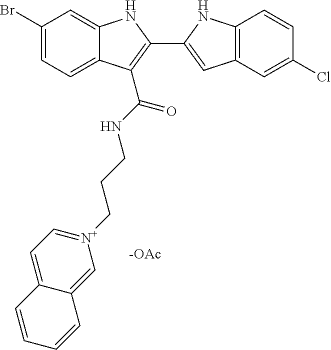 [SMILES: O=C(NCCC[n+]1ccc2ccccc2c1)c1c(-c2cc3cc(Cl)ccc3[nH]2)[nH]c2cc(Br)ccc12] |
| 147 | 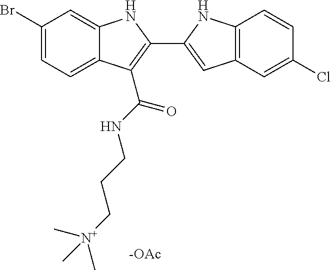 [SMILES: C[N+](C)(C)CCCNC(=O)c1c(-c2cc3cc(Cl)ccc3[nH]2)[nH]c2cc(Br)ccc12] |
| 148 | 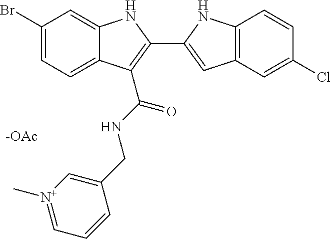 [SMILES: C[n+]1cccc(CNC(=O)c2c(-c3cc4cc(Cl)ccc4[nH]3)[nH]c3cc(Br)ccc23)c1] |
| 149 | 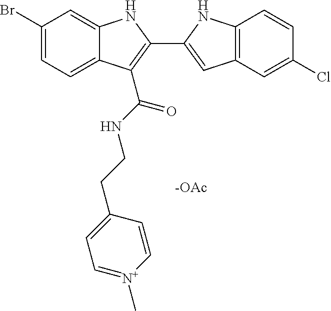 [SMILES: C[n+]1ccc(CCNC(=O)c2c(-c3cc4cc(Cl)ccc4[nH]3)[nH]c3cc(Br)ccc23)cc1] |
| 150 | 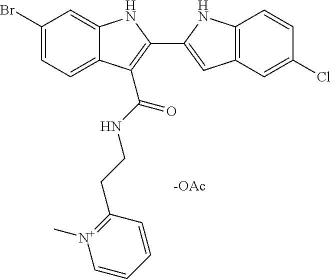 [SMILES: C[n+]1ccccc1CCNC(=O)c1c(-c2cc3cc(Cl)ccc3[nH]2)[nH]c2cc(Br)ccc12] |
| 151 | 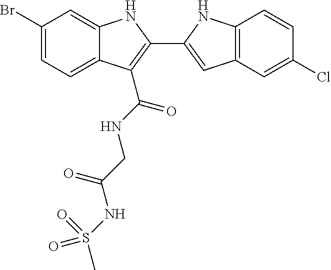 [SMILES: CS(=O)(=O)NC(=O)CNC(=O)c1c(-c2cc3cc(Cl)ccc3[nH]2)[nH]c2cc(Br)ccc12] |
| 152 | 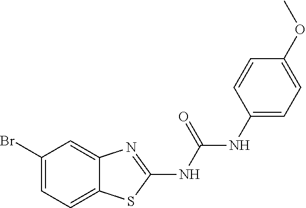 [SMILES: COc1ccc(NC(=O)Nc2nc3cc(Br)ccc3s2)cc1] |
| 153 | 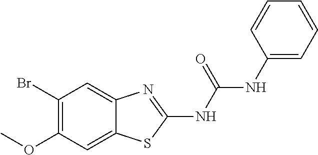 [SMILES: COc1cc2sc(NC(=O)Nc3ccccc3)nc2cc1Br] |
| 154 | 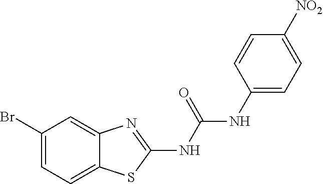 [SMILES: O=C(Nc1ccc([N+](=O)[O-])cc1)Nc1nc2cc(Br)ccc2s1] |
| 155 | 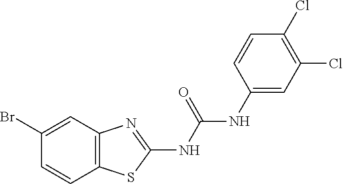 [SMILES: O=C(Nc1ccc(Cl)c(Cl)c1)Nc1nc2cc(Br)ccc2s1] |
| 156 | 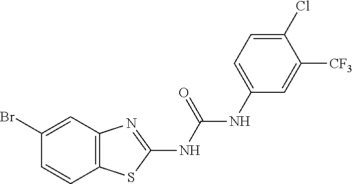 [SMILES: O=C(Nc1ccc(Cl)c(C(F)(F)F)c1)Nc1nc2cc(Br)ccc2s1] |
| 157 | 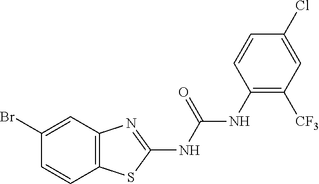 [SMILES: O=C(Nc1nc2cc(Br)ccc2s1)Nc1ccc(Cl)cc1C(F)(F)F] |
| 158 | 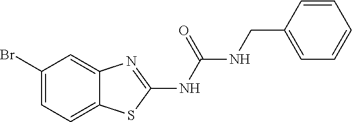 [SMILES: O=C(NCc1ccccc1)Nc1nc2cc(Br)ccc2s1] |
| 159 | 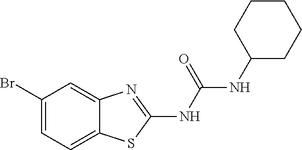 [SMILES: O=C(Nc1nc2cc(Br)ccc2s1)NC1CCCCC1] |
| 160 | 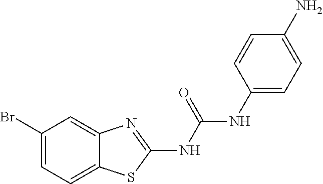 [SMILES: Nc1ccc(NC(=O)Nc2nc3cc(Br)ccc3s2)cc1] |
| 161 | 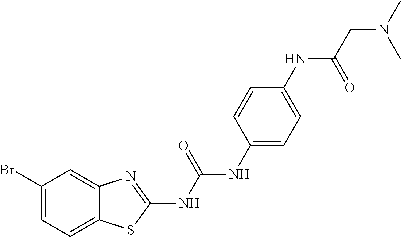 [SMILES: CN(C)CC(=O)Nc1ccc(NC(=O)Nc2nc3cc(Br)ccc3s2)cc1] |
| 162 | 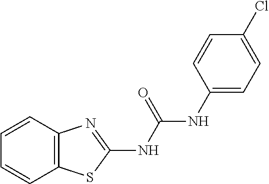 [SMILES: O=C(Nc1ccc(Cl)cc1)Nc1nc2ccccc2s1] |
| 163 | 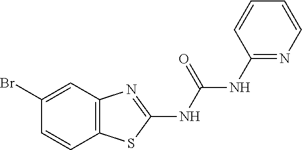 [SMILES: O=C(Nc1ccccn1)Nc1nc2cc(Br)ccc2s1] |
| 164 | 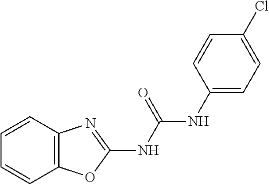 [SMILES: O=C(Nc1ccc(Cl)cc1)Nc1nc2ccccc2o1] |
| 165 | 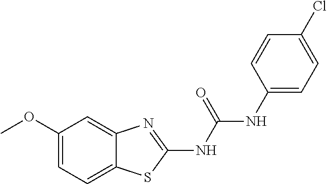 [SMILES: COc1ccc2sc(NC(=O)Nc3ccc(Cl)cc3)nc2c1] |
| 166 | 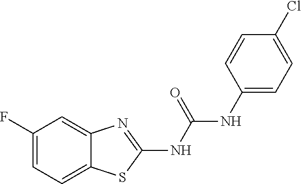 [SMILES: O=C(Nc1ccc(Cl)cc1)Nc1nc2cc(F)ccc2s1] |
| 167 | 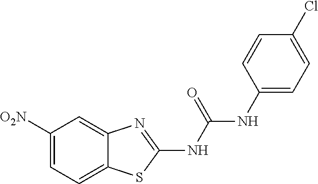 [SMILES: O=C(Nc1ccc(Cl)cc1)Nc1nc2cc([N+](=O)[O-])ccc2s1] |
| 168 | 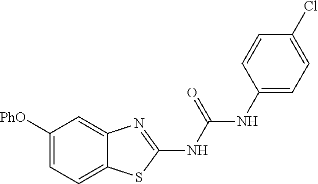 [SMILES: O=C(Nc1ccc(Cl)cc1)Nc1nc2cc(Oc3ccccc3)ccc2s1] |
| 169 | 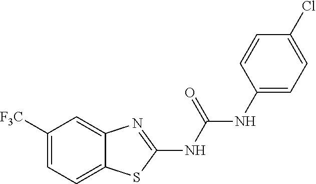 [SMILES: O=C(Nc1ccc(Cl)cc1)Nc1nc2cc(C(F)(F)F)ccc2s1] |
| 170 | 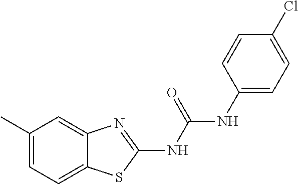 [SMILES: Cc1ccc2sc(NC(=O)Nc3ccc(Cl)cc3)nc2c1] |
| 171 | 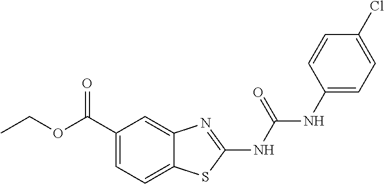 [SMILES: CCOC(=O)c1ccc2sc(NC(=O)Nc3ccc(Cl)cc3)nc2c1] |
| 172 | 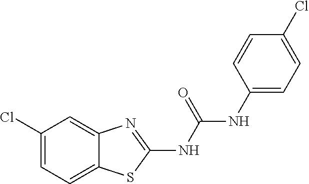 [SMILES: O=C(Nc1ccc(Cl)cc1)Nc1nc2cc(Cl)ccc2s1] |
| 173 | 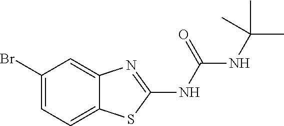 [SMILES: CC(C)(C)NC(=O)Nc1nc2cc(Br)ccc2s1] |
| 174 | 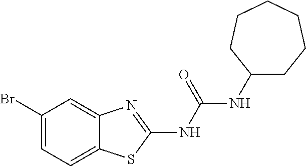 [SMILES: O=C(Nc1nc2cc(Br)ccc2s1)NC1CCCCCC1] |
| 175 | 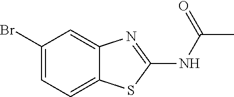 |
| 176 | 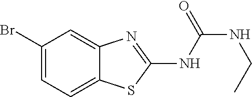 [SMILES: CCNC(=O)Nc1nc2cc(Br)ccc2s1] |
| 177 | 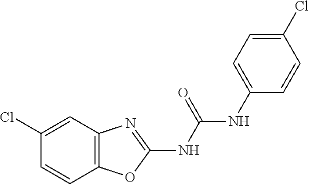 [SMILES: O=C(Nc1ccc(Cl)cc1)Nc1nc2cc(Cl)ccc2o1] |
| 178 | 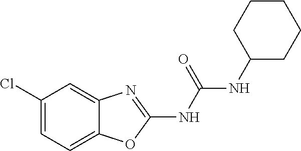 [SMILES: O=C(Nc1nc2cc(Cl)ccc2o1)NC1CCCCC1] |
| 179 | 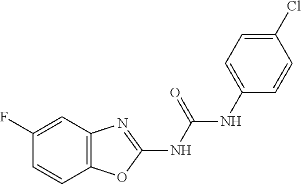 [SMILES: O=C(Nc1ccc(Cl)cc1)Nc1nc2cc(F)ccc2o1] |
| 180 | 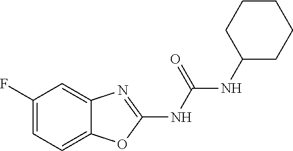 [SMILES: O=C(Nc1nc2cc(F)ccc2o1)NC1CCCCC1] |
| 181 | 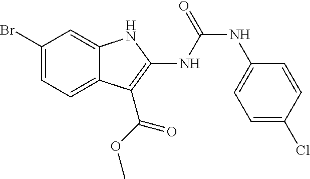 [SMILES: COC(=O)c1c(NC(=O)Nc2ccc(Cl)cc2)[nH]c2cc(Br)ccc12] |
| 182 | 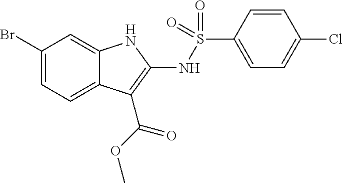 [SMILES: COC(=O)c1c(NS(=O)(=O)c2ccc(Cl)cc2)[nH]c2cc(Br)ccc12] |
| 183 | 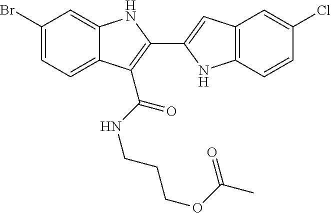 [SMILES: CC(=O)OCCCNC(=O)c1c(-c2cc3cc(Cl)ccc3[nH]2)[nH]c2cc(Br)ccc12] |
| 184 | 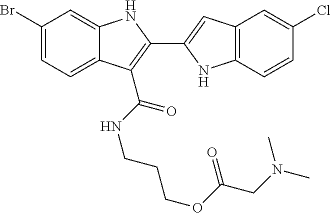 [SMILES: CN(C)CC(=O)OCCCNC(=O)c1c(-c2cc3cc(Cl)ccc3[nH]2)[nH]c2cc(Br)ccc12] |
| 185 | 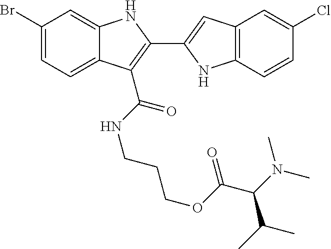 [SMILES: CC(C)[C@@H](C(=O)OCCCNC(=O)c1c(-c2cc3cc(Cl)ccc3[nH]2)[nH]c2cc(Br)ccc12)N(C)C] |
| 186 | 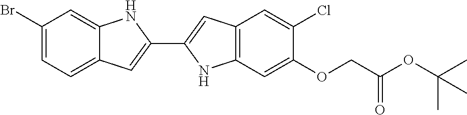 [SMILES: CC(C)(C)OC(=O)COc1cc2[nH]c(-c3cc4ccc(Br)cc4[nH]3)cc2cc1Cl] |
| 187 | 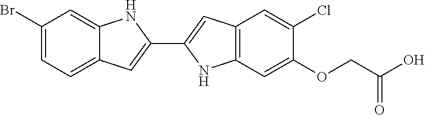 [SMILES: O=C(O)COc1cc2[nH]c(-c3cc4ccc(Br)cc4[nH]3)cc2cc1Cl] |
| 188 | 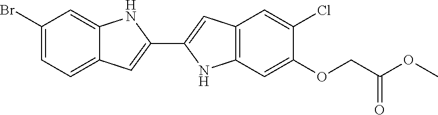 [SMILES: COC(=O)COc1cc2[nH]c(-c3cc4ccc(Br)cc4[nH]3)cc2cc1Cl] |
| 189 | 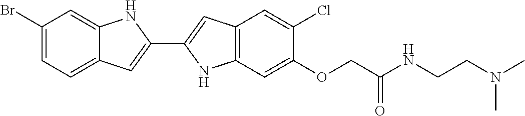 [SMILES: CN(C)CCNC(=O)COc1cc2[nH]c(-c3cc4ccc(Br)cc4[nH]3)cc2cc1Cl] |
| 190 | 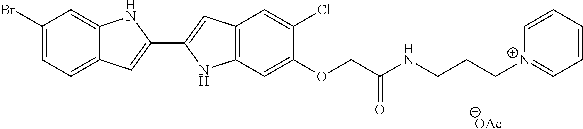 [SMILES: O=C(COc1cc2[nH]c(-c3cc4ccc(Br)cc4[nH]3)cc2cc1Cl)NCCC[n+]1ccccc1 ; "OAc"] |
| 191 | 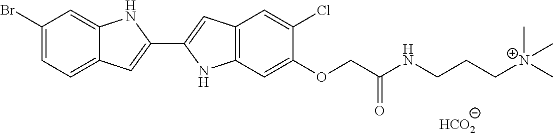 |
| 192 | 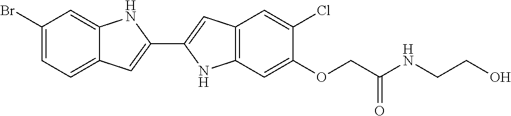 [SMILES: O=C(COc1cc2[nH]c(-c3cc4ccc(Br)cc4[nH]3)cc2cc1Cl)NCCO] |
| 193 | 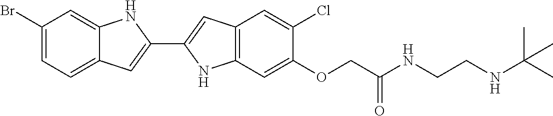 [SMILES: CC(C)(C)NCCNC(=O)COc1cc2[nH]c(-c3cc4ccc(Br)cc4[nH]3)cc2cc1Cl] |
| 194 | 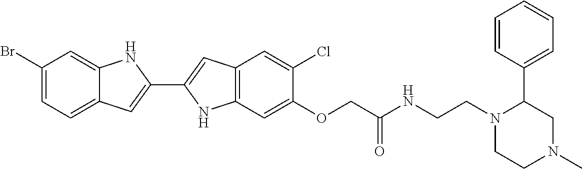 [SMILES: CN1CCN(CCNC(=O)COc2cc3[nH]c(-c4cc5ccc(Br)cc5[nH]4)cc3cc2Cl)C(c2ccccc2)C1] |
| 195 | 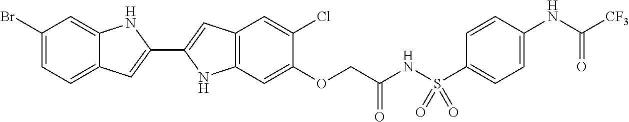 [SMILES: O=C(COc1cc2[nH]c(-c3cc4ccc(Br)cc4[nH]3)cc2cc1Cl)NS(=O)(=O)c1ccc(NC(=O)C(F)(F)F)cc1] |
| 196 | 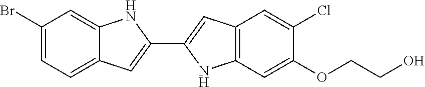 [SMILES: OCCOc1cc2[nH]c(-c3cc4ccc(Br)cc4[nH]3)cc2cc1Cl] |
| 197 | 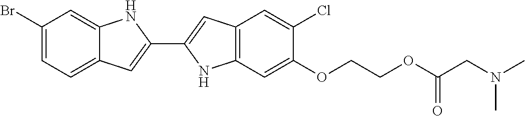 [SMILES: CN(C)CC(=O)OCCOc1cc2[nH]c(-c3cc4ccc(Br)cc4[nH]3)cc2cc1Cl] |
| 198 | 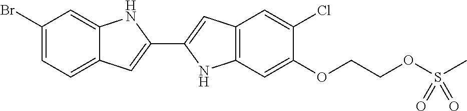 [SMILES: CS(=O)(=O)OCCOc1cc2[nH]c(-c3cc4ccc(Br)cc4[nH]3)cc2cc1Cl] |
| 199 | 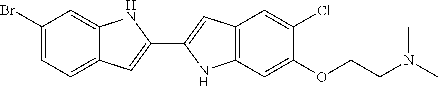 [SMILES: CN(C)CCOc1cc2[nH]c(-c3cc4ccc(Br)cc4[nH]3)cc2cc1Cl] |
| 200 | 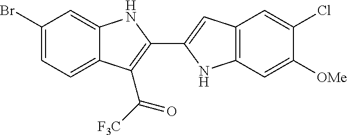 [SMILES: COc1cc2[nH]c(-c3[nH]c4cc(Br)ccc4c3C(=O)C(F)(F)F)cc2cc1Cl] |
| 201 | 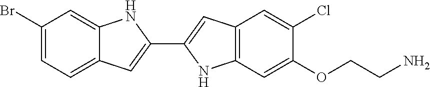 [SMILES: NCCOc1cc2[nH]c(-c3cc4ccc(Br)cc4[nH]3)cc2cc1Cl] |
| 202 | 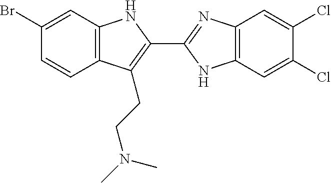 [SMILES: CN(C)CCc1c(-c2nc3cc(Cl)c(Cl)cc3[nH]2)[nH]c2cc(Br)ccc12] |
| 203 | 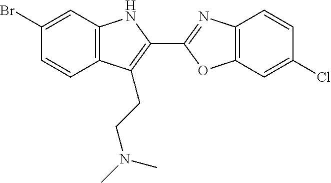 [SMILES: CN(C)CCc1c(-c2nc3ccc(Cl)cc3o2)[nH]c2cc(Br)ccc12] |
| 204 | 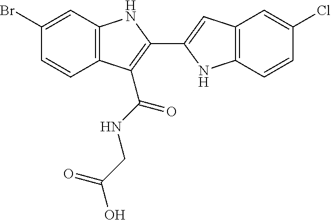 [SMILES: O=C(O)CNC(=O)c1c(-c2cc3cc(Cl)ccc3[nH]2)[nH]c2cc(Br)ccc12] |
| 205 | 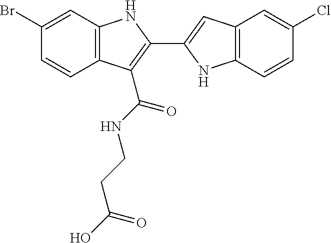 [SMILES: O=C(O)CCNC(=O)c1c(-c2cc3cc(Cl)ccc3[nH]2)[nH]c2cc(Br)ccc12] |
| 206 | 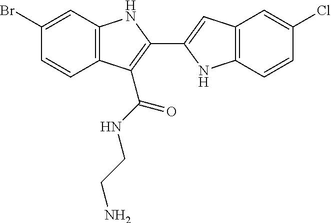 [SMILES: NCCNC(=O)c1c(-c2cc3cc(Cl)ccc3[nH]2)[nH]c2cc(Br)ccc12] |
| 207 | 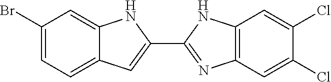 [SMILES: Clc1cc2nc(-c3cc4ccc(Br)cc4[nH]3)[nH]c2cc1Cl] |
| 208 | 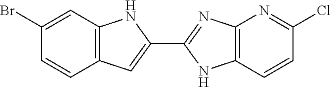 [SMILES: Clc1ccc2[nH]c(-c3cc4ccc(Br)cc4[nH]3)nc2n1] |
| 209 | 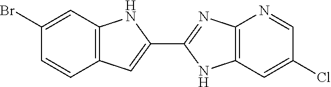 [SMILES: Clc1cnc2nc(-c3cc4ccc(Br)cc4[nH]3)[nH]c2c1] |
| 210 | 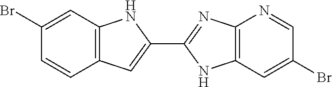 [SMILES: Brc1ccc2cc(-c3nc4ncc(Br)cc4[nH]3)[nH]c2c1] |
| 211 | 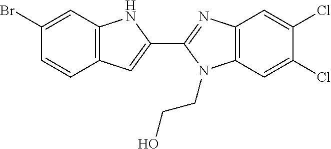 [SMILES: OCCn1c(-c2cc3ccc(Br)cc3[nH]2)nc2cc(Cl)c(Cl)cc21] |
| 212 | 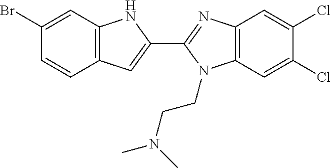 [SMILES: CN(C)CCn1c(-c2cc3ccc(Br)cc3[nH]2)nc2cc(Cl)c(Cl)cc21] |
| 213 | 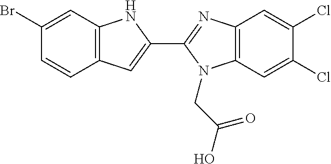 [SMILES: O=C(O)Cn1c(-c2cc3ccc(Br)cc3[nH]2)nc2cc(Cl)c(Cl)cc21] |
| 214 | 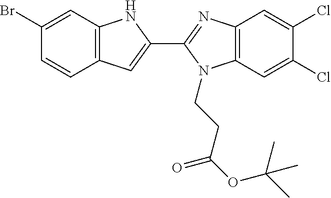 [SMILES: CC(C)(C)OC(=O)CCn1c(-c2cc3ccc(Br)cc3[nH]2)nc2cc(Cl)c(Cl)cc21] |
| 215 | 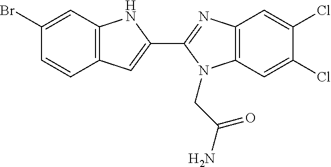 [SMILES: NC(=O)Cn1c(-c2cc3ccc(Br)cc3[nH]2)nc2cc(Cl)c(Cl)cc21] |
| 216 | 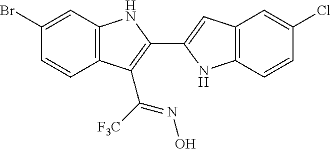 [SMILES: O/N=C(/c1c(-c2cc3cc(Cl)ccc3[nH]2)[nH]c2cc(Br)ccc12)C(F)(F)F] |
| 217 | 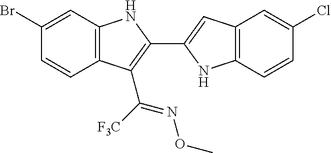 [SMILES: CO/N=C(/c1c(-c2cc3cc(Cl)ccc3[nH]2)[nH]c2cc(Br)ccc12)C(F)(F)F] |
| 218 | 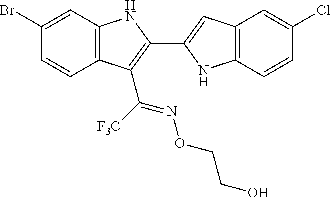 [SMILES: OCCO/N=C(/c1c(-c2cc3cc(Cl)ccc3[nH]2)[nH]c2cc(Br)ccc12)C(F)(F)F] |
| 219 | 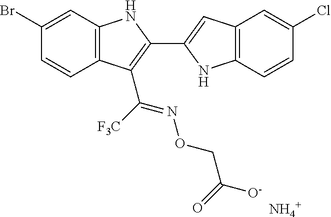 [SMILES: O=C([O-])CO/N=C(/c1c(-c2cc3cc(Cl)ccc3[nH]2)[nH]c2cc(Br)ccc12)C(F)(F)F ; [NH4+]] |
| 220 | 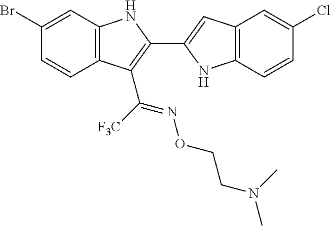 [SMILES: CN(C)CCO/N=C(/c1c(-c2cc3cc(Cl)ccc3[nH]2)[nH]c2cc(Br)ccc12)C(F)(F)F] |
| 221 | 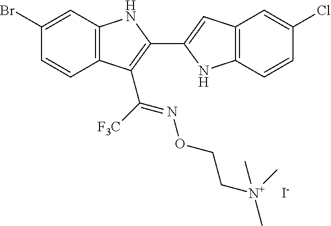 [SMILES: C[N+](C)(C)CCO/N=C(/c1c(-c2cc3cc(Cl)ccc3[nH]2)[nH]c2cc(Br)ccc12)C(F)(F)F ; [I-]] |
| 222 | 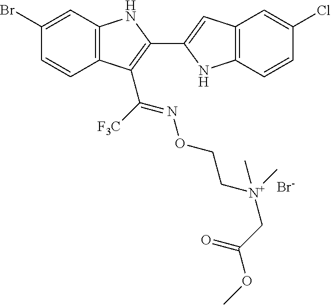 [SMILES: COC(=O)C[N+](C)(C)CCO/N=C(/c1c(-c2cc3cc(Cl)ccc3[nH]2)[nH]c2cc(Br)ccc12)C(F)(F)F ; [Br-]] |
| 223 | 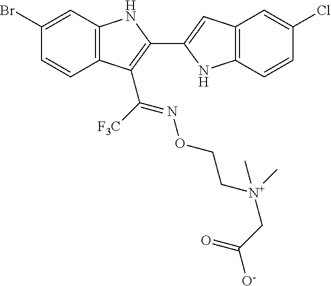 [SMILES: C[N+](C)(CCO/N=C(/c1c(-c2cc3cc(Cl)ccc3[nH]2)[nH]c2cc(Br)ccc12)C(F)(F)F)CC(=O)[O-]] |
| 224 | 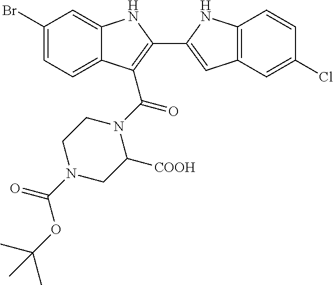 [SMILES: CC(C)(C)OC(=O)N1CCN(C(=O)c2c(-c3cc4cc(Cl)ccc4[nH]3)[nH]c3cc(Br)ccc23)C(C(=O)O)C1] |
| 225 | 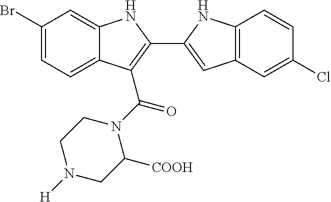 [SMILES: O=C(O)C1CNCCN1C(=O)c1c(-c2cc3cc(Cl)ccc3[nH]2)[nH]c2cc(Br)ccc12] |
| 226 | 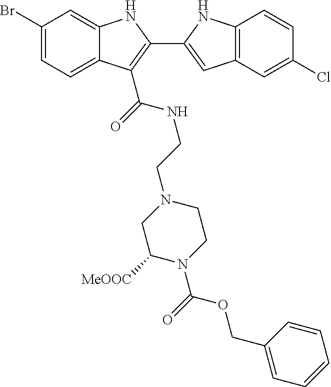 [SMILES: COC(=O)[C@@H]1CN(CCNC(=O)c2c(-c3cc4cc(Cl)ccc4[nH]3)[nH]c3cc(Br)ccc23)CCN1C(=O)OCc1ccccc1] |
| 227 | 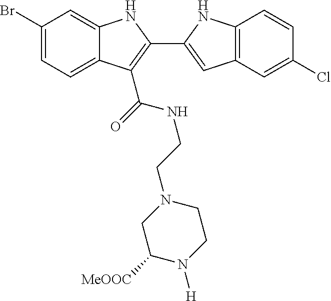 [SMILES: COC(=O)[C@@H]1CN(CCNC(=O)c2c(-c3cc4cc(Cl)ccc4[nH]3)[nH]c3cc(Br)ccc23)CCN1] |
| 228 | 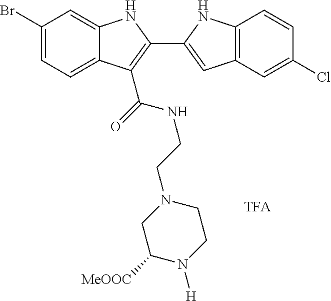 [SMILES: COC(=O)[C@@H]1CN(CCNC(=O)c2c(-c3cc4cc(Cl)ccc4[nH]3)[nH]c3cc(Br)ccc23)CCN1] |
| 229 | 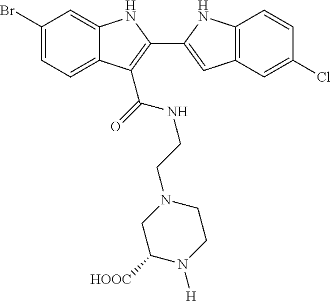 [SMILES: O=C(NCCN1CCN[C@H](C(=O)O)C1)c1c(-c2cc3cc(Cl)ccc3[nH]2)[nH]c2cc(Br)ccc12] |

or a salt thereof, wherein the compound, or salt thereof, has anti-bacterial activity.

67. A method of reducing the prefalence of bacteria on a surface, the method comprising introducing a compound according to any one of claims 1 to 33 to the surface.

68. Use a compound having a structure of formula (1): 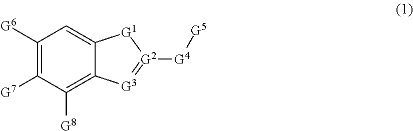
or a salt thereof,
wherein:
G¹ is NH, O, or S;
G², G³ and G⁴ may either: i) together form a ring moiety selected from the group consisting of: 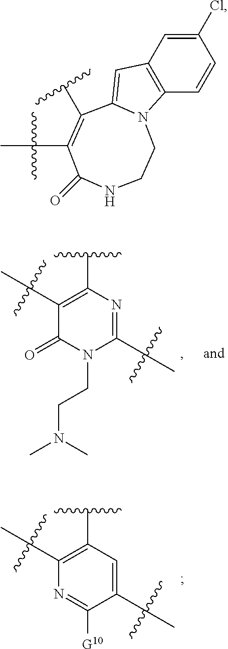
or
ii) together do not form a ring moiety wherein G² is C; G³ is N, CH or CG⁹; and
G⁴ is selected from the group consisting of: a bond, 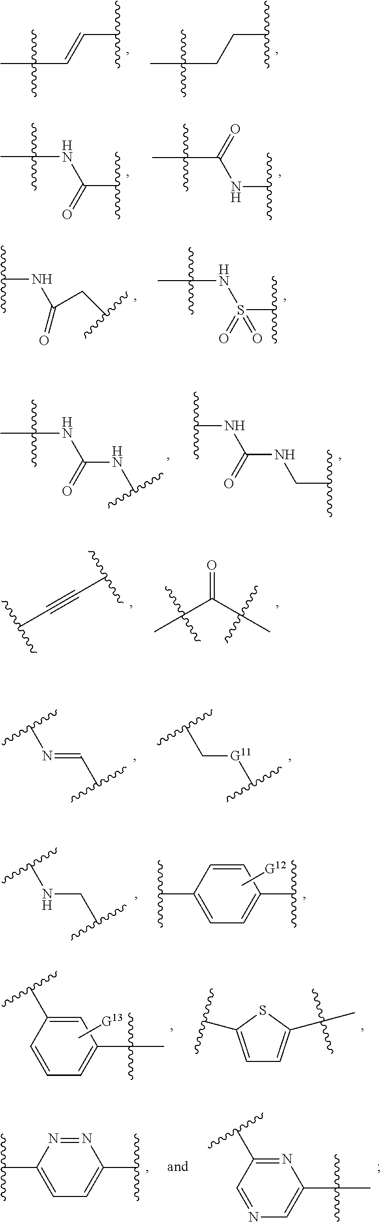
G⁵ is absent, 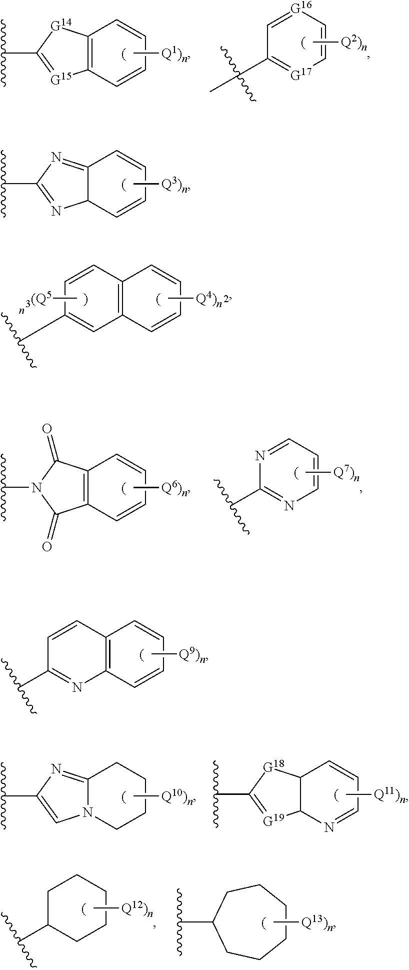
a 5-membered heteroaryl optionally substituted with (Q⁸)ₙ and containing 1 or 2 heteroatoms each heteroatom independently selected from N, O and S, substituted (C₁₋₁₁)alkyl, unsubstituted (C₁₋₁₁)alkyl, substituted (C₁₋₁₁)heteroalkyl, unsubstituted (C₁₋₁₁)heteroalkyl, substituted (C₃₋₁₁)heterocycloalkyl, unsubstituted (C₃₋₁₁)heterocycloalkyl, substituted (C₈₋₉)cycloalkyl, or unsubstituted (C₈₋₉)cycloalky;
G⁶ is H, halogen, CF₃, NO₂, substituted (C₁₋₁₁)alkyl, unsubstituted (C₁₋₁₁)alkyl, substituted (C₁₋₁₁)alkoxyl, unsubstituted (C₁₋₁₁) alkoxyl, substituted (C₆₋₁₁)aryloxy, unsubstituted (C₆₋₁₁)aryloxy, C(O)OR⁵⁰, substituted (C₁₋₁₁)heteroalkyl, unsubstituted (C₁₋₁₁) heteroalkyl or 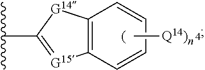
G⁷ is H, halogen, CF₃, NO₂, substituted (C₁₋₁₁)alkyl, unsubstituted (C₁₋₁₁)alkyl, substituted (C₁₋₁₁) alkoxyl, unsubstituted (C₁₋₁₁) alkoxy, substituted (C₆₋₁₁)aryloxy, unsubstituted (C₆₋₁₁)aryloxy, C(O)OR⁵¹, substituted (C₁₋₁₁)heteroalkyl, unsubstituted (C₁₋₁₁) heteroalkyl, or 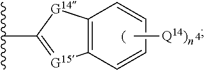
R⁵⁰ and R⁵¹ are each independently substituted (C₁₋₆)alkyl, unsubstituted (C₁₋₆)alkyl, substituted (C₁₋₆)heteroalkyl or unsubstituted (C₁₋₆) heteroalkyl;
G⁸ is H, C(═O)N(CH₃)₂, or C(═O)N(H)C(H₂)C₆H₅;
G⁹ is —CN, CF₃, —SO₂NH₂, —NH₂, —C(CF₃)₂OH, —C(CF₃)(H)OH, —C(CF₃)(CH₃)OH, —C(NOH)C(R²¹)(R²²)(R²³), C(NOH)N(R²⁴)(R²⁵), C(NOR⁶⁰)C(R⁶¹)(R⁶²)(R⁶³),
substituted (C₁₋₆) alkyl-NR⁶⁴R⁶⁵, unsubstituted (C₁₋₆) alkyl-NR⁶⁴R⁶⁵,
substituted (C₆₋₁₁) aryl, unsubstituted (C₆₋₁₁)aryl,
substituted (C₁₋₁₁) heteroaryl, unsubstituted (C₁₋₁₁) heteroaryl,
substituted (C₆₋₁₁) arylcarbonyl, unsubstituted (C₆₋₁₁) arylcarbonyl,
substituted (C₁₋₁₁) heteroarylcarbonyl, unsubstituted (C₁₋₁₁) heteroarylcarbonyl,
—CO-substituted-carbocycle, —CO-unsubstituted-carbocycle,
—CO-substituted-heterocarbocycle, —CO-unsubstituted-heterocarbocycle,
—CO-substituted-C(₁₋₆)alkyl-OR¹, —CO-unsubstituted-C(₁₋₆)alkyl-OR¹,
—CO-substituted-C(₁₋₆)alkyl-NR²R³, —CO-unsubstituted-C(₁₋₆)alkyl-NR²R³,
—CO-substituted-C(₁₋₆)alkyl-C(O)OR⁴, —CO-unsubstituted-C(₁₋₆)alkyl-C(O)OR⁴;
—CO-substituted-C(₁₋₆)alkyl-C(O)NR⁵R⁶, —CO-unsubstituted-C(₁₋₆)alkyl-C(O)NR⁵R⁶,
—C(O)NR⁷R⁸, —C(O)OR⁹, —C(O)C(O)OR¹², —C(O)C(O)NR¹³R¹⁴, —NR¹⁵R¹⁶,
—N(H)C(O)substituted-C(₁₋₆)alkyl, —N(H)C(O)unsubstituted-C(₁₋₆)alkyl,
—N(H)C(O)substituted-C(₁₋₆)haloalkyl, —N(H)C(O)unsubstituted-C(₁₋₆)haloalkyl,
—N(H)C(O)substituted-C(₆₋₁₁)aryl, —N(H)C(O)unsubstituted-C(₆₋₁₁)aryl,
—N(H)C(O)substituted-C(₁₋₁₁)heteroaryl, —N(H)C(O)unsubstituted-C(₁₋₁₁)heteroaryl,
—N(H)C(O)NR¹⁷R¹⁸,
—N(H)CO-substituted-C(₁₋₆)alkyl-OR¹⁹, —N(H)CO-unsubstituted-C(₁₋₆)alkyl-OR¹⁹,
each of R¹, R², R³, R⁴, R⁵, R⁶, R¹², R¹³, R¹⁴, R¹⁵, R¹⁶, R¹⁷, R¹⁸, R¹⁹, R²⁴, and R²⁵ is independently selected from the group consisting of: H, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl,
each of R²¹, R²², R²³, R⁶¹, R⁶² and R⁶³ is independently selected from the group consisting of: H, F, substituted C(₁₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl;
each of R⁶⁴ and R⁶⁵ is independently selected from the group consisting of: H, substituted C(₃₋₆)alkyl, substituted C(₁₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₁₋₁₁)aryl, unsubstituted C(₁₋₁₁)heteroaryl, unsubstituted C(₇₋₁₁)aralkyl, and unsubstituted C(₂₋₁₁)heteroaralkyl
each pair: a) R² and R³, b) R⁵ and R⁶, c) R¹³ and R¹⁴, d) R¹⁵ and R¹⁶, e) R¹⁷ and R¹⁸, and f) R⁶⁴ and R⁶⁵ may alternately be and independently as a pair be a 3-7 membered substituted heterocarbocyclic ring or a 3-7 membered unsubstituted heterocarbocyclic ring;
R⁶⁰ is unsubstituted C(₁₋₁₁)alkyl, substituted C(₁₋₁₁)alkyl, unsubstituted C(₁₋₁₁)alkyl-NR⁶⁶R⁶⁷, substituted C(₁₋₁₁)alkyl-NR⁶⁶R⁶⁷, unsubstituted C(₁₋₁₁)alkyl-N⁺R⁶⁸R⁶⁹R⁷⁰, or substituted C(₁₋₁₁)alkyl-N⁺R⁶⁸R⁶⁹R⁷⁰, wherein R⁶⁶ and R⁶⁷ are each independently H, unsubstituted C(₁₋₁₁)alkyl or substituted C(₁₋₁₁)alkyl, and R⁶⁸, R⁶⁹ and R⁷⁰ are each independently unsubstituted C(₁₋₁₁)alkyl, or substituted C(₁₋₁₁)alkyl,
each of R⁷ and R⁸ are either
I) independently selected from the group consisting of: H, substituted C(₁₋₆)alkyl, substituted C(₁₋₆)alkyl-NR⁵²R⁵³, unsubstituted C(₁₋₆)alkyl-NR⁵²R⁵³, substituted C(₁₋₆)alkyl-N⁺R⁷¹R⁷²R⁷³, unsubstituted C(₁₋₆)alkyl-N⁺R⁷¹R⁷²R⁷³, substituted C(₁₋₆)alkyl-OC(O)unsubstituted C(₁₋₆)alkyl-NR⁷⁴R⁷⁵, unsubstituted C(₁₋₆)alkyl-OC(O)unsubstituted C(₁₋₆)alkyl-NR⁷⁴R⁷⁵, substituted C(₁₋₆)alkyl-C(O)NHS(O)₂R⁷⁶, unsubstituted C(₁₋₆)alkyl-C(O)NHS(O)₂R⁷⁶, substituted C(₆₋₁₁)aryl, substituted C(₃₋₁₁)carbocyclic, substituted C(₄₋₇)heterocarbocycle, substituted C(₄₋₇)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₃₋₁₁)carbocyclic, unsubstituted C(₁₋₁₁)heterocarbocycle, unsubstituted C(₁₋₁₁)heteroaryl, unsubstituted C(₇₋₁₁)aralkyl, and unsubstituted C(₂₋₁₁)heteroaralkyl wherein each of R⁵², R⁵³, R⁷⁴ and R⁷⁵ is selected from the group consisting of: H, unsubstituted C(₁₋₆)alkyl, substituted C(₃₋₇)heterocycloalkyl, unsubstituted C(₃₋₇)heterocycloalkyl, substituted C(₁₋₆)alkyl, substituted C(₃₋₇)cycloalkyl and unsubstituted C(₃₋₇)cycloalkyl, or each pair: a) R⁵² and R⁵³, or (b) R⁷⁴ and R⁷⁵, together form a 3-7 membered substituted heterocarbocyclic ring or a 3-7 membered unsubstituted heterocarbocyclic ring, and wherein each of R⁷¹, R⁷², R⁷³ and R⁷⁶ is independently unsubstituted C(₁₋₁₁)alkyl, or substituted C(₁₋₁₁)alkyl, or
II) together form a 3-7 membered substituted heterocarbocyclic ring or a 3-7 membered unsubstituted heterocarbocyclic ring;
R⁹ is selected from the group consisting of substituted C(₁₋₆)alkyl, substituted C(₁₋₆)alkyl-NR¹⁰R¹¹, unsubstituted C(₁₋₆)alkyl-NR¹⁰R¹¹, substituted C(₁₋₆)alkyl-OR²⁰, unsubstituted C(₁₋₆)alkyl-OR²⁰, and unsubstituted C(₁₋₆)alkyl wherein each of R¹⁰, R¹¹, and R²⁰ is independently selected from the group consisting of: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; R¹⁰ and R¹¹ may alternately as a pair be a 3-7 membered substituted heterocarbocyclic ring or a 3-7 membered unsubstituted heterocarbocyclic ring, or
G⁹ is 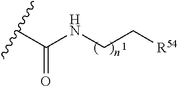
wherein n¹ is 1, 2, 3 or 4 and R⁵⁴ is 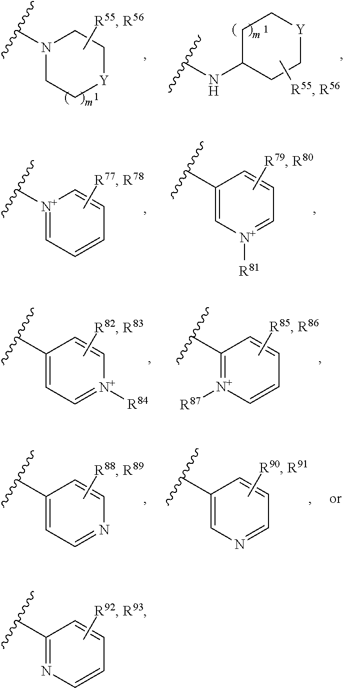
wherein m¹=0, 1 or 2, R⁵⁵ and R⁵⁶ are independently H, carbonyl (═O), Me, Ph, CO₂R⁹⁴, CO₂NH₂, C(₁₋₆)substituted alkyl or C(₁₋₆)unsubstituted alkyl, wherein R⁹⁴ is H, C(₁₋₆)unsubstituted alkyl or C(₁₋₆)substituted alkyl;
R⁷⁷, R⁷⁸, R⁷⁹, R⁸⁰, R⁸², R⁸³, R⁸⁵, R⁸⁶, R⁸⁸, R⁸⁹, R⁹⁰, R⁹¹, R⁹² and R⁹³ are each independently H, C(₁₋₆)substituted alkyl, C(₁₋₆)unsubstituted alkyl, substituted C(₁₋₆)heteroalkyl, unsubstituted C₍₁₋₆₎ heteroalkyl, OR⁹⁵, C(O)R⁹⁶, or NR⁹⁷R⁹⁸, wherein R⁹⁵, is H, C(₁₋₆)substituted alkyl, or C(₁₋₆)unsubstituted alkyl, R⁹⁶ is C(₁₋₆)substituted alkyl, or C(₁₋₆)unsubstituted alkyl, and R⁹⁷ and R⁹⁸ are each independently H, C(₁₋₆)substituted alkyl, or C(₁₋₆)unsubstituted alkyl, or each pair: a) R⁷⁷ and R⁷⁸, b) R⁷⁹ and R⁸⁰, c) R⁸² and R⁸³, d) R⁸⁵ and R⁸⁶, e) R⁸⁸ and R⁸⁹, f) R⁹⁰ and R⁹¹, or g) R⁹² and R⁹³ are attached to adjacent ring-forming C atoms, and together with the ring-forming C atoms, form a substituted C₆ aryl ring or an unsubstituted C₆ aryl ring;
R⁸¹, R⁸⁴ and R⁸⁷ each independently is C(₁₋₆)substituted alkyl, or C(₁₋₆)unsubstituted alkyl; and
Y is CH₂, CHOH, CHO—CO—C(₁₋₆)unsubstituted alkyl, CHO—CO—C(₁₋₆)substituted alkyl, NCONH₂, N—C(₁₋₆)substituted alkyl, N—C(₁₋₆)unsubstituted alkyl, NH or N—C(O)OR⁹⁹, wherein R⁹⁹ is C(₁₋₆)unsubstituted alkyl, C(₁₋₆)substituted alkyl, C(₆₋₁₁)unsubstituted aralkyl or C(₆₋₁₁)substituted aralkyl;
G¹⁰ is selected from the group consisting of: a straight C(₁₋₆)alkyl, a branched C(₃₋₆)alkyl and phenyl;
G¹¹ is NHCH₂, NH, NHCO, SCH₂, O, or S;
G¹² is H, NO₂, or OMe;
G¹³ is H, NO₂, or OMe;
each of G¹⁴, G^{14′} and G¹⁸ is independently NH, S, O, N—CH₃, N—CH₂—OCH₃, N—CH₂—COOH, N—CH₂—CH₂OH, N—CH₂—C(O)NH₂, CH—CH₃, N—R^{14′}, CH—R^{14′} or substituted C(₁₋₆)alkyl-NR⁵²R⁵³, wherein R^{14′} is C₍₁₋₆₎ substituted alkyl, C₍₁₋₆₎ unsubstituted alkyl, 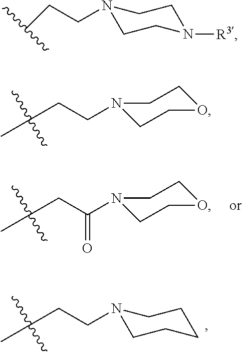
wherein R^{3′} is H, unsubstituted alkyl, or substituted alkyl, wherein the alkyl is 1-6, carbons in length, and the alkyl is optionally substituted with Br, F, Cl, I, OH, OMe, or N₃;
each of G¹⁵, G^{15′} and G¹⁹ is independently N, CH or CG⁹;
G¹⁶ is N or CH;
G¹⁷ is N or CH;
each of n, n², n³ and n⁴ is independently 0, 1, 2, 3, or 4;
each Q¹ and Q¹⁴ is independently selected from the group consisting of: halogen, —OR²⁶, —O—(C₁₋₆)alkyl-NR²⁷R²⁸, —O—(C₁₋₆)alkyl-C(O)OR¹⁰⁰, —O—(C₁₋₆)alkyl-C(O)NHR¹⁰¹, —O—(C—O—(C₁₋₆)alkyl-OC(O)R¹⁰², —O—(C₁₋₆)alkyl-OS(O)₂R¹⁰³, NO₂, NR¹⁰⁴R¹⁰⁵, —NHC(O)R¹⁰⁶, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q² is independently selected from the group consisting of: halogen, —OR²⁹, —O—(C₁₋₆)alkyl-NR³⁰R³¹, —O—(C₁₋₆)alkyl-C(O)OR¹⁰⁷, —O—(C₁₋₆)alkyl-C(O)NHR¹⁰⁸, —O—(C₁₋₆)alkyl-OC(O)R¹⁰⁹, —O—(C₁₋₆)alkyl-OS(O)₂R¹¹⁰, NO₂, NR¹¹¹R¹¹², —NHC(O)R¹¹³, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q³ is independently selected from the group consisting of: halogen, —OR¹¹⁴, —O—(C₁₋₆)alkyl-NR¹¹⁵R¹¹⁶, —O—(C₁₋₆)alkyl-C(O)OR¹¹⁷, —O—(C₁₋₆)alkyl-C(O)NHR¹¹⁸, —O—(C₁₋₆)alkyl-OC(O)R¹¹⁹, —O—(C₁₋₆)alkyl-OS(O)₂R¹²⁰, NO₂, NR¹²¹R¹²², —NHC(O)R¹²³, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q⁴ is independently selected from the group consisting of: halogen, —OR³⁵, —O—(C₁₋₆)alkyl-NR³⁶R³⁷, —O—(C₁₋₆)alkyl-C(O)OR¹²⁴, —O—(C₁₋₆)alkyl-C(O)NHR¹²⁵, —O—(C₁₋₆)alkyl-OC(O)R¹²⁶, —O—(C₁₋₆)alkyl-OS(O)₂R¹²⁷, NO₂, NR¹²⁸R¹²⁹, —NHC(O)R¹³⁰, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q⁵ is independently selected from the group consisting of: halogen, —OR³⁸, —O—(C₁₋₆)alkyl-NR³⁹R⁴⁰, —O—(C₁₋₆)alkyl-C(O)OR¹³¹, —O—(C₁₋₆)alkyl-C(O)NHR¹³², —O—(C₁₋₆)alkyl-OC(O)R¹³³, —O—(C₁₋₆)alkyl-OS(O)₂R¹³⁴, NO₂, NR¹³⁵R¹³⁶, —NHC(O)R¹³⁷, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q⁶ is independently selected from the group consisting of: halogen, —OR⁴¹, —O—(C₁₋₆)alkyl-NR⁴²R⁴³, —O—(C₁₋₆)alkyl-C(O)OR¹³⁸, —O—(C₁₋₆)alkyl-C(O)NHR¹³⁹, —O—(C₁₋₆)alkyl-OC(O)R¹⁴⁰, —O—(C₁₋₆)alkyl-OS(O)₂R¹⁴¹, NO₂, NR¹⁴²R¹⁴³, —NHC(O)R¹⁴⁴, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q⁷ is independently selected from the group consisting of: halogen, —OR⁴⁴, —O—(C₁₋₆)alkyl-NR⁴⁵R⁴⁶, —O—(C₁₋₆)alkyl-C(O)OR¹⁴⁵, —O—(C₁₋₆)alkyl-C(O)NHR¹⁴⁶, —O—(C₁₋₆)alkyl-OC(O)R¹⁴⁷, —O—(C₁₋₆)alkyl-OS(O)₂R¹⁴⁸, NO₂, NR¹⁴⁹R¹⁵⁰, —NHC(O)R¹⁵¹, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q⁸ is independently selected from the group consisting of: halogen, —OR⁴⁷, —O—(C₁₋₆)alkyl-NR⁴⁸R⁴⁹, —O—(C₁₋₆)alkyl-C(O)OR¹⁵², —O—(C₁₋₆)alkyl-C(O)NHR¹⁵³, —O—(C₁₋₆)alkyl-OC(O)R¹⁵⁴, —O—(C₁₋₆)alkyl-OS(O)₂R¹⁵⁵, NO₂, NR¹⁵⁶R¹⁵⁷, —NHC(O)R¹⁵⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q⁹ is independently selected from the group consisting of: halogen, —OR¹⁵⁹, —O—(C₁₋₆)alkyl-NR¹⁶⁰R¹⁶¹, —O—(C₁₋₆)alkyl-C(O)OR¹⁶², —O—(C₁₋₆)alkyl-C(O)NHR¹⁶³, —O—(C₁₋₆)alkyl-OC(O)R¹⁶⁴, —O—(C₁₋₆)alkyl-OS(O)₂R¹⁶⁵, NO₂, NR¹⁶⁶R¹⁶⁷, —NHC(O)R¹⁶⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q¹⁰ is independently selected from the group consisting of:
halogen, —OR¹⁶⁹, —O—(C₁₋₆)alkyl-NR¹⁷⁰R¹⁷¹, —O—(C₁₋₆)alkyl-C(O)OR¹⁷², —O—(C₁₋₆)alkyl-C(O)NHR¹⁷³, —O—(C₁₋₆)alkyl-OC(O)R¹⁷⁴, —O—(C₁₋₆)alkyl-OS(O)₂R¹⁷⁵, NO₂, NR¹⁷⁶R¹⁷⁷, —NHC(O)R¹⁷⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q¹¹ is independently selected from the group consisting of:
halogen, —OR¹⁷⁹, —O—(C₁₋₆)alkyl-NR¹⁸⁰R¹⁸¹, —O—(C₁₋₆)alkyl-C(O)OR¹⁸², —O—(C₁₋₆)alkyl-C(O)NHR¹⁸³, —O—(C₁₋₆)alkyl-OC(O)R¹⁸⁴, —O—(C₁₋₆)alkyl-OS(O)₂R¹⁸⁵, NO₂, NR¹⁸⁶R¹⁸⁷, —NHC(O)R¹⁸⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q¹² is independently selected from the group consisting of:
halogen, —OR¹⁸⁹, —O—(C₁₋₆)alkyl-NR¹⁹⁰R¹⁹¹, —O—(C₁₋₆)alkyl-C(O)OR¹⁹², —O—(C₁₋₆)alkyl-C(O)NHR¹⁹³, —O—(C₁₋₆)alkyl-OC(O)R¹⁹⁴, —O—(C₁₋₆)alkyl-OS(O)₂R¹⁹⁵, NO₂, NR¹⁹⁶R¹⁹⁷, —NHC(O)R¹⁹⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each Q¹³ is independently selected from the group consisting of:
halogen, —OR¹⁹⁹, —O—(C₁₋₆)alkyl-NR²⁰⁰R²⁰¹, —O—(C₁₋₆)alkyl-C(O)OR²⁰², —O—(C₁₋₆)alkyl-C(O)NHR²⁰³, —O—(C₁₋₆)alkyl-OC(O)R²⁰⁴, —O—(C₁₋₆)alkyl-OS(O)₂R²⁰⁵, NO₂, NR²⁰⁶R²⁰⁷, —NHC(O)R²⁰⁸, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
each R²⁶, R²⁷, R²⁸, R²⁹, R³⁰, R³¹, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R¹⁰⁰, R¹⁰⁴, R¹⁰⁵, R¹⁰⁷, R¹¹¹, R¹¹², R¹¹⁴, R¹¹⁵, R¹¹⁶, R¹¹⁷, R¹²¹, R¹²², R¹²⁴, R¹²⁸, R¹²⁹, R¹³¹, R¹³⁵, R¹³⁶, R¹³⁸, R¹⁴², R¹⁴³, R¹⁴⁵, R¹⁴⁹, R¹⁵⁰, R¹⁵², R¹⁵⁶, R¹⁵⁷, R¹⁵⁹, R¹⁶⁰, R¹⁶¹, R¹⁶², R¹⁶⁶, R¹⁶⁷, R¹⁶⁹, R¹⁷⁰, R¹⁷¹, R¹⁷², R¹⁷⁶, R¹⁷⁷, R¹⁷⁹, R¹⁸⁰, R¹⁸¹, R¹⁸², R¹⁸⁶, R¹⁸⁷, R¹⁸⁹, R¹⁹⁰, R¹⁹¹, R¹⁹², R¹⁹⁶, R¹⁹⁷, R¹⁹⁹, R²⁰⁰, R²⁰¹, R²⁰², R²⁰⁶ and R²⁰⁷ are independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and each pair: a) R²⁷ and R²⁸, b) R³⁰ and R³¹, c) R³⁶ and R³⁷, d) R³⁹ and R⁴⁰, e) R⁴² and R⁴³, f) R⁴⁵ and R⁴⁶, g) R⁴⁸ and R⁴⁹, h) R¹⁰⁴ and R¹⁰⁵, i) R¹¹¹ and R¹¹², j) R¹¹⁵ and R¹¹⁶, k) R¹²¹ and R¹²², l) R¹²⁸ and R¹²⁹, m) R¹³⁵ and R¹³⁶, n) R¹⁴² and R¹⁴³, o) R¹⁴⁹ and R¹⁵⁰, p) R¹⁵⁶ and R¹⁵⁷, q) R¹⁶⁰ and R¹⁶¹, r) R¹⁶⁶ and R¹⁶⁷, s) R¹⁷⁰ and R¹⁷¹, t) R¹⁷⁶ and R¹⁷⁷, u) R¹⁸⁰ and R¹⁸¹, v) R¹⁸⁶ and R¹⁸⁷, w) R¹⁹⁰ and R¹⁹¹, x) R¹⁹⁶ and R¹⁹⁷, y) R²⁰⁰ and R²⁰¹, and z) R²⁰⁶ and R²⁰⁷ may alternately be and independently as a pair be a 3-7 membered substituted heterocarbocyclic ring or a 3-7 membered unsubstituted heterocarbocyclic ring;
R¹⁰¹, R¹⁰⁸, R¹¹⁸, R¹²⁵, R¹³², R¹³⁹, R¹⁴⁶, R¹⁵³, R¹⁶³, R¹⁷³, R¹⁸³, R¹⁹³ and R²⁰³, are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, unsubstituted C(₂₋₁₁)heteroaralkyl, substituted C(₁₋₆)alkyl-NR²⁰⁹R²¹⁰, unsubstituted C(₁₋₆)alkyl-NR²⁰⁹R²¹⁰, substituted C(₁₋₆)alkyl-N⁺R²¹¹R²¹²R²¹³, unsubstituted C(₁₋₆)alkyl-N⁺R²¹¹R²¹²R²¹³, substituted C(₁₋₆)alkyl-OR²¹⁴, unsubstituted C(₁₋₆)alkyl-OR²¹⁴, 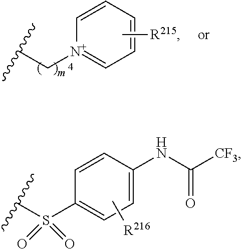
wherein m⁴ is 1, 2, 3, 4 or 5, R²⁰⁹, R²¹⁰, R²¹⁴, R²¹⁵ and R²¹⁶ are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and R²⁰⁹ and R²¹⁰, may alternately be and independently as a pair be a 3-7 membered substituted heterocarbocyclic ring or a 3-7 membered unsubstituted heterocarbocyclic ring, and R²¹¹, R²¹² and R²¹³ are each independently unsubstituted C(₁₋₁₁)alkyl, or substituted C(₁₋₁₁)alkyl; and
R¹⁰², R¹⁰³, R¹⁰⁶, R¹⁰⁹, R¹¹⁰, R¹¹³, R¹¹⁹, R¹²⁰, R¹²³, R¹²⁶, R¹²⁷, R¹³⁰, R¹³³, R¹³⁴, R¹³⁷, R¹⁴⁰, R¹⁴¹, R¹⁴⁴, R¹⁴⁷, R¹⁴⁸, R¹⁵¹, R¹⁵⁴, R¹⁵⁵, R¹⁵⁸, R¹⁶⁴, R¹⁶⁵, R¹⁶⁸, R¹⁷⁴, R¹⁷⁵, R¹⁷⁸, R¹⁸⁴, R¹⁸⁵, R¹⁸⁸, R¹⁹⁴, R¹⁹⁵, R¹⁹⁸, R²⁰⁴, R²⁰⁵ and R²⁰⁸ are each independently substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl;
(i) provided that G⁵ is absent only when G², G³ and G⁴ together form the ring moiety 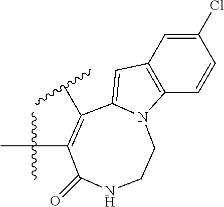 [SMILES: */C1=C(\*)c2cc3cc(Cl)ccc3n2CCNC1=O]
and G⁵ is absent when G², G³ and G⁴ together form the ring moiety 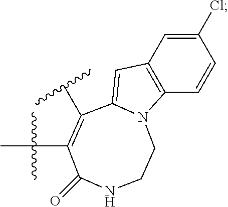 [SMILES: */C1=C(\*)c2cc3cc(Cl)ccc3n2CCNC1=O]
(ii) provided that when G³ is N, CH, or CG⁹ where G⁹ is C(O)OR⁹ and R⁹ is unsubstituted C₍₁₋₆₎ alkyl, G⁴ is other than 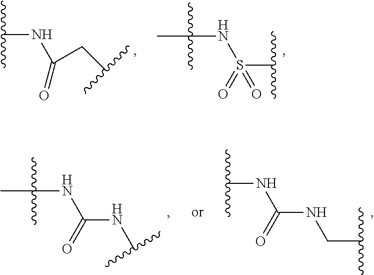 [SMILES: *CC(=O)N* ; *NS(*)(=O)=O ; *NC(=O)N* ; *CNC(=O)N*]
and G⁵ is 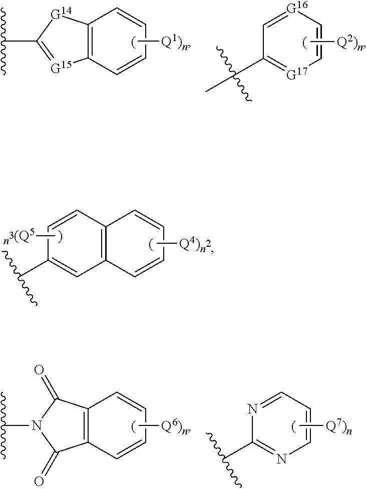
or a 5-membered heteroaryl optionally substituted with (Q⁸)ₙ and containing 1 or 2 heteroatoms each heteroatom independently selected from N, O and S, then n is at least 1 or n²+n³ is at least 1, and
(a) when n is 1 or n²+n³=1, then Q¹, Q², Q⁴, Q⁵, Q⁶, Q⁷ or Q⁸ is independently selected from the group consisting of —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NR^{104′}R^{105′}, and —NHC(O)R^{106′},
wherein R^{26′} is independently selected from the group consisting of substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₂₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl;
each R^{27′}, R^{28′}, R^{100′}, R^{104′} and R^{105′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; or each pair: a) R^{27′} and R^{28′}, or b) R^{104′} and R^{105′} may alternately be and independently as a pair be a 3-7 membered substituted heterocarbocyclic ring or a 3-7 membered unsubstituted heterocarbocyclic ring;
R^{101′} is H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, unsubstituted C(₂₋₁₁)heteroaralkyl, substituted C(₁₋₆)alkyl-NR^{209′}R^{210′}, unsubstituted C(₁₋₆)alkyl-NR^{209′}R^{210′}, substituted C(₁₋₆)alkyl-N⁺R^{211′}R^{212′}R^{213′}, unsubstituted C(₁₋₆)alkyl-N⁺R^{211′}R^{212′}R^{213′}, substituted C(₁₋₆)alkyl-OR^{214′}, unsubstituted C(₁₋₆)alkyl-OR^{214′}, 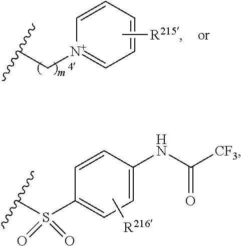
wherein m^{4′} is 1, 2, 3, 4 or 5, R^{209′}, R^{210′}, R^{214′}, R^{215′} and R^{216′} are each independently H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl or unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and R^{209′} and R^{210′}, may alternately be and independently as a pair be a 3-7 membered substituted heterocarbocyclic ring or a 3-7 membered unsubstituted heterocarbocyclic ring, and R^{211′}, R^{212′} and R^{213′} are each independently unsubstituted C(₁₋₁₁)alkyl, or substituted C(₁₋₁₁)alkyl; and
R^{102′}, R^{103′}, and R^{106′} are each independently substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₁₁)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and
(b) when n is at least 2 or n²+n³ is at least 2, then a first Q¹, Q², Q⁴, Q⁵, Q⁶, Q⁷ or Q⁸ is independently selected from the group consisting of —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NR^{104′}R^{105′}, and —NHC(O)R^{106′},
wherein each of R^{26′}, R^{27′}, R^{28′}, R^{100′}, R^{101′}, R^{102′}, R^{103′}, R^{104′}, R^{105′}, and R^{106′} is as defined above; and
the remaining Q¹, Q², Q⁴, Q⁵, Q⁶, Q⁷ or Q⁸ are each independently selected from the group consisting of halogen, —OR^{26′}, —O—(C₁₋₆)alkyl-NR^{27′}R^{28′}, —O—(C₁₋₆)alkyl-C(O)OR^{100′}, —O—(C₁₋₆)alkyl-C(O)NHR^{101′}, —O—(C₁₋₆)alkyl-OC(O)R^{102′}, —O—(C₁₋₆)alkyl-OS(O)₂R^{103′}, NO₂, NR^{104′}R^{105′}, —NHC(O)R^{106′}, substituted C₍₁₋₆₎alkyl, substituted C₍₁₋₆₎heteroalkyl, unsubstituted C₍₁₋₆₎alkyl, and unsubstituted C₍₁₋₆₎heteroalkyl;
wherein each R^{26′} is independently selected from the group consisting: H, substituted C(₁₋₆)alkyl, substituted C(₆₋₁₁)aryl, substituted C(₁₋₁₁)heteroaryl, substituted C(₇₋₁₁)aralkyl, substituted C(₂₋₁₁)heteroaralkyl, unsubstituted C(₁₋₆)alkyl, unsubstituted C(₆₋₁₁)aryl, unsubstituted C(₁₋₁₁)heteroaryl, unsubstituted C(₇₋₁₁)aralkyl, and unsubstituted C(₂₋₁₁)heteroaralkyl; and
each of R^{27′}, R^{28′}, R^{100′}, R^{101′}, R^{102′}, R^{103′}, R^{104′}, R^{105′}, and R^{106′} is as defined above; and
(iii) provided that when G³ is N, CH, or CG⁹ where G⁹ is C(O)OR⁹ and R⁹ is unsubstituted C₍₁₋₆₎ alkyl, G⁴ is other than 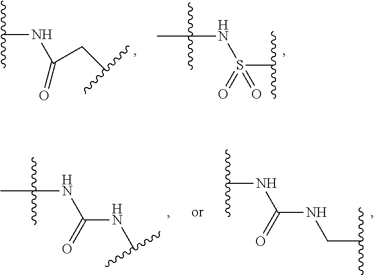 [SMILES: *CC(=O)N* ; *NS(*)(=O)=O ; *NC(=O)N* ; *CNC(=O)N*]
and G⁵ is 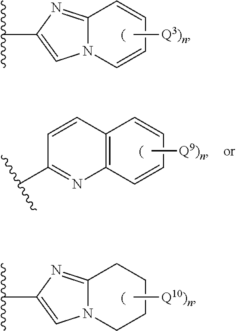
then n is at least 1
wherein each of Q³, Q⁹ and Q¹⁰ is as defined above,
for treatment of a bacterial infection.

69. Use of a compound according to any one of claims 1 to 33 for treatment of a bacterial infection.

70. Use of a compound according to any one of claims 1 to 33 for preparation of a medicament for treatment of a bacterial infection.


SEQUENCE LISTING (4 sequences)

SEQ ID NO: 1 - DNA, 29 bp, Artificial Sequence
  Primer
ctacatatgagaaaaactaaaattgtatg

SEQ ID NO: 2 - DNA, 33 bp, Artificial sequence
  Primer
gttctcgagttatagtacgtttgcatatccttc

SEQ ID NO: 3 - DNA, 33 bp, Artificial sequence
  Primer
gatcatatgatgtcgaagccccatagtgaagcc

SEQ ID NO: 4 - DNA, 34 bp, Artificial sequence
  Primer
gttctcgagtcacggcacaggaacaacacgcatg